(19) United States
(12) Patent Application Publication
(10) Pub. No.: US 2010/0041891 A1
(43) Pub. Date: Feb. 18, 2010

(54) AMIDE COMPOUND

(75) Inventors: Masaki Setoh, Osaka-shi (JP); Mitsunori Kouno, Osaka (JP); Yuhei Miyanohana, Osaka (JP); Masakuni Kori, Osaka (JP)

Correspondence Address: EDWARDS ANGELL PALMER & DODGE LLP, P.O. BOX 55874, BOSTON, MA, 02205, US

(73) Assignee: Takeda Pharmaceutical Company Limited, Osaka-Shi (JP)

(21) Appl. No.: 12/539,559

(22) Filed: Aug. 11, 2009

(30) Foreign Application Priority Data
Aug. 12, 2008 (JP) 2008-208260
May 28, 2009 (JP) 2009-129582

(51) Int. Cl. C07D 409/06; C07D 235/24; C07D 277/62

(52) U.S. Cl. 546/281.1; 548/306.4; 548/180

(57) ABSTRACT

A compound having GPR52 agonist activity or a salt thereof is provided.

The compound can be provided as a preventive/therapeutic agent for schizophrenia or the like. The compound is represented by the following formula:

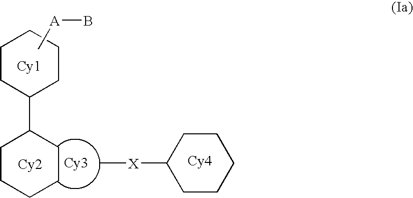

wherein

A represents —CONR^{a}— or —NR^{a}CO—,
R^{a} represents a hydrogen atom or the like,
B represents a hydrogen atom or the like,
a ring Cy1 represents a six-membered aromatic ring which may have one or more substituents in addition to a group represented by -A-B,
a ring Cy2 represents a six-membered ring which may be substituted with a halogen atom or the like,
a ring Cy3 represents a five- or six-membered ring which may have one or more substituents;
X represents C₁₋₂ alkylene or the like,
m represents an integer of 0 to 2, and
a ring Cy4 represents a six-membered aromatic ring which may have one or more substituents.

### TECHNICAL FIELD

The present invention relates to a novel amide compound and a method for manufacturing the same, and a pharmaceutical agent containing such a novel amide compound. More specifically, the present invention relates to a compound having an agonist effect on GPR52, which is effective as a pharmaceutical agent for preventing and treating mental disorders, such as schizophrenia, and the like.

### BACKGROUND OF THE INVENTION

Schizophrenia is a disease that occurs in people from adolescence to adulthood and shows characteristic thinking disturbances, disturbances of ego, and behavioral abnormalities associated therewith. The onset of symptoms is allegedly about 1% of the entire population. Most of them are chronic, so that the initiative or interpersonal contact of patients may be decreased, thereby interfering the social lives of the patients. The core symptoms of schizophrenia are broadly classified into (1) positive symptoms such as delusions and hallucination, (2) negative symptoms such as hypesthesia, social withdrawal, diminished motivation, and loss of concentration, and (3) cognitive dysfunction. In these core symptoms, the expression of positive symptoms is intimately involved in over activity of the dopamine nervous system in the mesolimbic system. The expression of the negative symptoms and impaired cognitive function are intimately involved in deterioration of the nervous system such as the glutamic acid nervous system in the cortex of frontal lobe.

In addition, a typical antipsychotic agent having an antagonist action on a dopamine D2 receptor, such as chlorpromazine, has favorable effects on the positive symptoms. On the other hand, drugs effective to multiple receptors, such as clozapine and olanzapine have certain effects on negative symptoms and impaired cognitive function. However, it is known that many patients have poor response on these drugs. Also, the typical antipsychotic agent has controversial side effects such as the occurrence of extrapyramidal syndromes, for example akathisia, dystonia, and Parkinson-like movement disorders and the occurrence of hyperprolactinemia. Furthermore, clozapine may cause agranulocytosis as a grave side effect. An atypical antipsychotic agent such as olanzapine may cause side effects, such as weight gain, lipidosis, excessive sedative effect, and prolonged cardiac QT interval.

Human GPR52 (Sawzdargo et al., Molecular Brain Research, 64: 193-198, 1999) has been known as one of G protein-coupled receptors (GPCRs). In recent years, because of an increase in cellular cAMP level in nerve cells expressing GPR52 or the like, any of agonists and antagonists against GPR52 has been considered to have an effect of improving the negative symptoms of schizophrenia by suppressing the hyperactivation of dopamine pathway in the mesolimbic region, one of the causes of the positive symptoms of schizophrenia. In addition, it has been also found that the agonists and antagonists against GPR52 can improve the negative symptoms of schizophrenia and cognitive deficiency by an improvement in decreased function of NMDA receptors in the cerebral cortex, which has been considered as one of the causes of such troubles (WO 2006/098520).

Therefore, it has been demanded to develop a compound having an agonistic effect on GPR52 and useful as a preventive/therapeutic pharmaceutical agent for mental diseases such as schizophrenia.

On the other hand, amide compounds with fused rings have been disclosed in several documents and examples thereof are as follows:

(1) International Publication WO 2007/117607 pamphlet discloses PDK1 inhibitors, which include a fused-ring amide compound, represented by the following general formula:

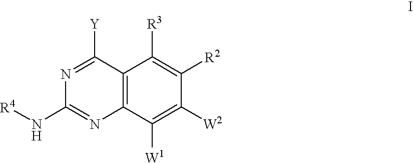

(2) International Publication WO 2006/116412 pamphlet discloses CRF receptor antagonists, which include the fused-ring amide compound, represented by the following general formula:

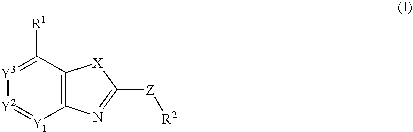

(3) International Publication WO 2005/061484 pamphlet discloses δ-receptor ligands, which include the fused-ring amide compound, represented by the following general formula:

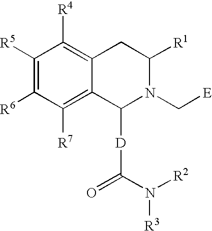

(4) International Publication WO 2006/138695 pamphlet discloses cannabinoid receptor I antagonists, which include the fused-ring amide compound, represented by the following general formula:

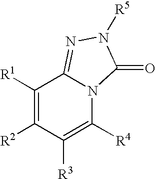

(5) U.S. Patent Application Publication No. 2005/143381 discloses cannabinoid modulators, which include the fused-ring amide compound, represented by the following general formula:

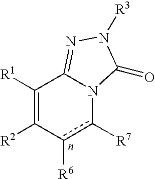

(6) International Publication WO 2001/083476 pamphlet discloses antimicrobial agents, which include the fused-ring amide compound, represented by the following general formula:

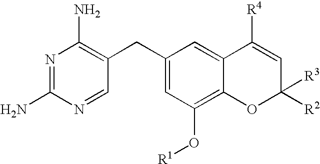

### [Citation List]

### [Patent Literature]

### [PTL 1]

International Publication WO 2006/098520 pamphlet

### [PTL 2]

International Publication WO 2007/117607 pamphlet

### [PTL 3]

International Publication WO 2006/116412 pamphlet

### [PTL 4]

International Publication WO 2005/061484 pamphlet

### [PTL 5]

International Publication WO 2006/138695 pamphlet

### [PTL 6]

International Publication WO 2005/143381 pamphlet

### [PTL 7]

International Publication WO 2001/083476 pamphlet

### [Non Patent Literature]

### [NPL 1]

Sawzdargo et al., Molecular Brain Research, 64: pp. 193-198, 1999.

### SUMMARY OF INVENTION

### Technical Problem

An object of the present invention is to provide a compound having an agonistic effect on GPR52 and useful as a preventive/therapeutic pharmaceutical agent for mental diseases such as schizophrenia.

### Solution to Problem

The present inventors have found that compounds represented by the below formula (I₀) or salts thereof (herein also referred to as compounds (I₀)) have an agonistic effect on GPR52 and finally completed the present invention by further investigations.

Furthermore, among the compounds (I₀), compounds represented by the below formula (Ia) and the formula (I) or a salt thereof (herein also referred to as compound (Ia) and compound (I)) are novel compounds.

The compound (I₀) including the compound (Ia) and compound (I) or prodrugs thereof will be herein also referred to as the compounds of the present invention.

In other words, the present invention provides the following [1] to [42] and the like. [1] A compound represented by formula (Ia):

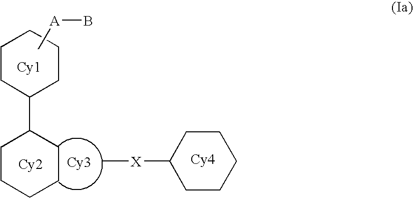

wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
   a halogen atom,
   a cyano group,
   a hydroxy group,
   a hydrocarbon-oxy group which may have one or more substituents,
   a chain hydrocarbon group which may have one or more substituents (except for a methyl group substituted with a five-membered heterocyclic group),
   a heterocyclic group which may have one or more substituents,
   an amino group which may have one or more substituents,
   an acyl group, and
   a carboxy group which may be esterified;
ring Cy3 represents a five- or six-membered ring which may have one or more substituents;
X represents a C₁₋₂ alkylene which may be substituted with hydroxy, —Y—, Y—CH₂—, or —CH₂—Y—;
Y represents —O—, —NR^{b}—, or —S(O)ₘ—;
R^{b} represents a hydrogen atom or a substituent;
m represents an integer of 0 to 2; and
ring Cy4 represents a six-membered aromatic ring which may have one or more substituents (except for a sulfamoyl group which may have one or more substituents;
with the proviso that a compound represented by the following formula:

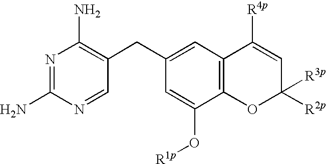

wherein
R^{1p} represents alkyl or cycloalkylalkyl;
R^{2p} and R^{3p} each independently represent an alkyl or a cycloalkyl or represent, together with an adjacent carbon atom, any of saturated three- to six-membered carbon rings or heterocyclic rings (where alkyl, cycloalkyl, a carbon ring, or a heterocyclic ring is unsaturated or saturated), and
R^{4p} represents aryl which may be substituted or heteroaryl which may be substituted, a compound represented by the following formula:

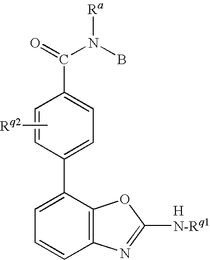

wherein
R^{q1} represents phenyl which may have one or more substituents,
R^{q2} represents hydrogen, or a substituent,
the other symbols are synonymous with those described above, a compound represented by the following formula:

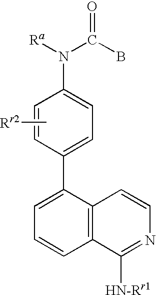

wherein
R^{r1} represents phenyl which may have one or more substituents,
R^{q2} represents hydrogen, or a substituent, the other symbols are synonymous with those described above,
7-[4-(acetylamino)phenyl]-2-(benzylsulfanyl)-5-methyl-N-phenyl-3,7-dihydro[1,2,4]triazolo[1,5-a]pyrimidine-6- carboxamide,
7-[4-(acetylamino)phenyl]-2-[(4-chlorobenzyl)sulfanyl]-N-(2,4-dimethylphenyl)-5-methyl-3,7-dihydro[1,2,4]triazolo [1,5-a]pyrimidine-6-carboxamide,
7-[4-(acetylamino)phenyl]-2-(benzylsulfanyl)-N-(2-methoxyphenyl)-5-methyl-3,7-dihydro[1,2,4]triazolo[1,5-a]pyrimidine-6-carboxamide,
7-[4-(acetylamino)phenyl]-2-[(2,4-dimethylbenzyl) sulfanyl]-N-(4-methoxyphenyl)-5-methyl-3,7-dihydro[1,2,4]triazolo[1,5-a]pyrimidine-6-carboxamide,
7-[4-(acetylamino)phenyl]-2-(benzylsulfanyl)-N-(2,4-dimethylphenyl)-5-methyl-3,7-dihydro[1,2,4]triazolo [1,5-a]pyrimidine-6-carboxamide,
N-(3-(2-((4-chloro-2-methoxy-6-methylphenyl)amino)-1-methyl-1H-benzoimidazol-7-yl)phenyl)acetamide),
tert-butyl methyl[4-[2-methyl-1-(phenylsulfonyl)-1H-pyrrolo[2,3-b]pyridin-4-yl]phenyl]carbamate,
tert-butyl (4-[3-[(4-methoxybenzyl)amino]imidazo[1,5-a]pyridin-5-yl]phenyl)carbamate,
tert-butyl 1-[4-(diethylcarbamoyl)phenyl]-6-methoxy-7-phenoxy-3,4-dihydroisoquinoline-2(1H)-carboxylate,
tert-butyl 1-[4-(diethylcarbamoyl)phenyl]-7-(4-fluorophenoxy)-6-methoxy-3,4-dihydroisoquinoline-2(1H)-carboxylate,
tert-butyl 1-[4-(diethylcarbamoyl)phenyl]-6-methoxy-7-(4-methoxyphenoxy)-3,4-dihydroisoquinoline-2(1H)-carboxylate,
tert-butyl 1-[4-(diethylcarbamoyl)phenyl]-6-methoxy-7-(pyridin-3-yloxy)-3,4-dihydroisoquinolie-2(1H)-carboxylate,

1-[4-(1-benzyl-1H-pyrazolo[3,4-c]pyridin-4-yl)phenyl]-3-[3-(trifluoromethyl)phenyl]urea,
1-[4-[1-(4-methoxybenzyl)-1H-pyrazolo[3,4-c]pyridin-4-yl]phenyl]-3-[3-(trifluoromethyl) phenyl]urea,
3-chloro-2-[6-[(2-chloro-4-fluorophenyl)sulfanyl]-2-oxo-3,4-dihydropyrido[3,2-d]pyrimidin-1(2H)-yl]benzamide,
3,5-dichloro-4-[6-[(2,4-difluorophenyl)sulfanyl]-2-oxo-3,4-dihydropyrido[3,2-d]pyrimidin-1(2H)-yl]benzamide,
3,5-dichloro-4-[6-[(2,4-difluorophenyl)sulfanyl]-2-oxo-3,4-dihydropyrido[3,2-d]pyrimidin-1(2H)-yl]-N-[2-(dimethylamino)ethyl]benzamide,
2-chloro-N-(3-chloro-4-[2-[(4-fluorophenyl)sulfanyl]-6-oxo-7,8-dihydro-6H-pyrimido[1,6-b]pyridazin-5-yl]phenyl)acetamide,
N-(3-chloro-4-[2-[(4-fluorophenyl)sulfanyl]-6-oxo-7,8-dihydro-6H-pyrimido[1,6-b]pyridazin-5-yl]phenyl)acetamide,
N-(3-chloro-4-[2-[(4-fluorophenyl)sulfanyl]-6-oxo-7,8-dihydro-6H-pyrimido[1,6-b]pyridazin-5-yl]phenyl)-2-morpholin-4-ylacetamide,
N-(4-[2-[(3,4,5-trimethoxyphenyl)amino]-1,3-benzoxazol-7-yl]phenyl)acetamide,
N-(3-[2-[(3,4,5-trimethoxyphenyl)amino]-1,3-benzoxazol-7-yl]phenyl)acetamide,
N-(2-amino-4-[2-[(3,4,5-trimethoxyphenyl)amino]-1,3-benzoxazol-7-yl]phenyl)formamide,
7-[4-(acetylamino)phenyl]-2-[(2,4-dimethylbenzyl)sulfanyl]-5-methyl-N-phenyl-3,7-dihydro[1,2,4]triazolo[1,5-a]pyrimidine-6-carboxamide,
5-[(3S)-3-(dibenzylamino)-3,4-dihydro-2H-chromen-5-yl]-2-methoxy-N,N-dimethylpyridine-3-carboxamide,
5-[(3S)-3-(dibenzylamino)-3,4-dihydro-2H-chromen-5-yl]-2-methoxy-N-methylpyridine-3-carboxamide,
5-[(3S)-3-(dibenzylamino)-3,4-dihydro-2H-chromen-5-yl]-2-methoxy-N-methylpyridine-3-carboxamide, and
N-(6-[1-[(4-methylphenyl)sulfonyl]-1H-pyrrolo[2,3-b]pyridin-4-yl]pyridin-2-yl)acetamide, are excluded;
or a salt thereof.

[2] A compound represented by formula (I):

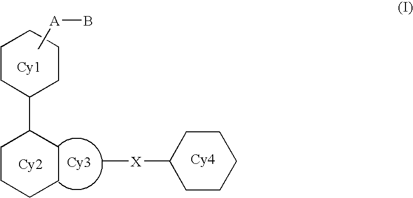

wherein
A represents —CONR^{a}— or —NR^{a}CO—,
R^{a} represents a hydrogen atom or a substituent,
B represents a hydrogen atom or a substituent,
or alternatively, when A is —CONR^{a}—, R^{a} and B may form together with an adjacent nitrogen atom a nitrogen-containing heterocyclic group which may have one or more substituents;
ring Cy1 represents a six-membered aromatic ring which may have one or more substituents in addition to a group represented by -A-B;
ring Cy2 represents a six-membered ring which may have one or more substituents selected from
   a halogen atom,
   a cyano group,
   a hydroxy group,
   a hydrocarbon-oxy group which may have one or more substituents,
   a chain hydrocarbon group which may have one or more substituents (except for a methyl group substituted with a five-membered heterocyclic group),
   a heterocyclic group which may have one or more substituents,
   an amino group which may have one or more substituents,
   an acyl group, and
   a carboxy group which may be esterified;
ring Cy3 represents a five- or six-membered ring which may have one or more substituents;
X represents a C₁₋₂ alkylene, —Y—, Y—CH₂—, or —CH₂—Y—;
Y represents —O—, —NR^{b}, or —S(O)ₘ—;
R^{b} represents a hydrogen atom or a substituent;
m represents an integer of 0 to 2; and
ring Cy4 represents a six-membered aromatic ring which may have one or more substituents (except for a sulfamoyl group which may have one or more substituents;
with the proviso that a compound represented by the following formula:

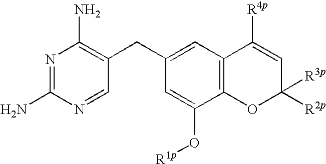

wherein
R^{1p} represents alkyl or cycloalkylalkyl;
R^{2p} and R^{3p} each independently represent an alkyl or a cycloalkyl or represent, together with an adjacent carbon atom, any of saturated three- to six-membered carbon rings or heterocyclic rings (where alkyl, cycloalkyl, a carbon ring, or a heterocyclic ring is unsaturated or saturated), and
R^{4p} represents aryl which may be substituted or heteroaryl which may be substituted,
7-[4-(acetylamino)phenyl]-2-(benzylsulfanyl)-5-methyl-N-phenyl-3,7-dihydro[1,2,4]triazolo[1,5-a]pyrimidine-6- carboxamide,
7-[4-(acetylamino)phenyl]-2-[(4-chlorobenzyl)sulfanyl]-N-(2,4-dimethylphenyl)-5-methyl-3,7-dihydro[1,2,4]triazolo [1,5-a]pyrimidine-6-carboxamide,
7-[4-(acetylamino)phenyl]-2-(benzylsulfanyl)-N-(2-methoxyphenyl)-5-methyl-3,7-dihydro[1,2,4]triazolo[1,5-a]pyrimidine-6-carboxamide,
7-[4-(acetylamino)phenyl]-2-[(2,4-dimethylbenzyl) sulfanyl]-N-(4-methoxyphenyl)-5-methyl-3,7-dihydro[1,2,4]triazolo[1,5-a]pyrimidine-6-carboxamide,
7-[4-(acetylamino)phenyl]-2-(benzylsulfanyl)-N-(2,4-dimethylphenyl)-5-methyl-3,7-dihydro[1,2,4]triazolo [1,5-a]pyrimidine-6-carboxamide, and
N-(3-(2-((4-chloro-2-methoxy-6-methylphenyl)amino)-1-methyl-1H-benzoimidazol-7-yl)phenyl)acetamide) are excluded;
or a salt thereof.

[3] The compound according to the above [2], wherein ring Cy1 is a benzene ring or a pyridine ring.

[4] The compound according to the above [2], wherein ring Cy2 is a six-membered ring which may have one or more substituents selected from a halogen atom, an alkyl group which may have one or more substituents, and an alkoxy group which may have one or more substituents.

[5] The compound according to the above [2], wherein ring Cy3 is a five- or six-membered ring which may have one or more substituents selected from a halogen atom, an alkyl group which may have one or more substituents, and an alkoxy group which may have one or more substituents.

[6] The compound according to the above [2], wherein ring Cy4 is a benzene ring or a pyridine ring, which may have one or more substituents a substituent (except for a sulfamoyl group which may have one or more substituents).

[7] The compound according to the above [2], wherein the chemical formula (I) is as follows:

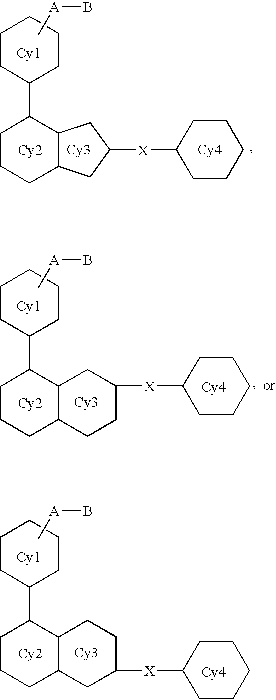

[8] The compound according to the above [2], wherein the chemical formula (I) is as follows:

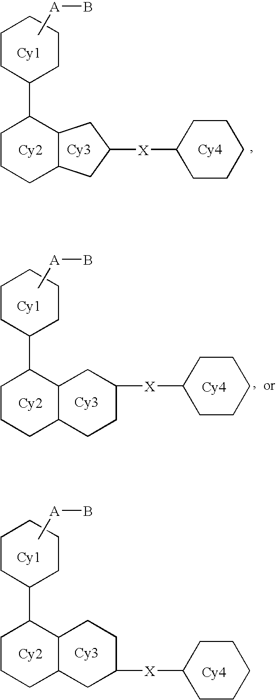

ring Cy1 is a benzene ring or a pyridine ring;

ring Cy2 is a six-membered ring which may have one or more substituents selected from a halogen atom, an alkyl group which may have one or more substituents, and an alkoxy group which may have one or more substituents;

ring Cy3 is a five- or six-membered ring which may have one or more substituents selected from a halogen atom, an alkyl group which may have one or more substituents, and an alkoxy group which may have one or more substituents; and

ring Cy 4 is a benzene ring or a pyridine ring, which may have one or more substituents.

[9] The compound according to the above [1], wherein the chemical formula (Ia) is as follows:

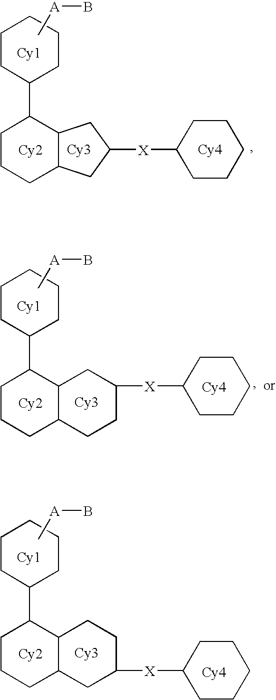

A represents —CONR^{a}— or —NR^{a}CO—;
R^{a} represents a hydrogen atom or a C₁₋₆ alkyl group a substituent;
B represents
   1) a hydrogen atom,
   2) a C₁₋₆ alkyl group which may have one or more substituents selected from
      a) cyano group,
      b) a hydroxy group,
      c) a C₁₋₆ alkoxy group,
      d) a C₆₋₁₄ aryloxy group,
      e) a carbamoyl group, and
      f) an amino group which may be substituted with one or two substitutes selected from a C₁₋₆ alkyl group, a C₆₋₁₄ aryl group, a C₁₋₆ alkylcarbonyl group,
      g) a C₆₋₁₄ aryl group which may be substituted with an amino group which may be substituted with one or two C₁₋₆ alkyl group,
      h) a five- or six-membered heterocyclic group which may be substituted with a substituent selected from a C₁₋₆ alkyl group and an oxo group,
      i) a C₁₋₆ alkylsulfanyl group,
      j) a C₁₋₆ alkylsulfinyl group, and
      k) a C₁₋₆ alkylsulfonyl group,
      3) a C₃₋₆ cycloalkyl group which may be substituted with a hydroxy group,
      4) a C₆₋₁₄ aryl group which may be substituted with a five- or six-membered heterocyclic group, or
      5) a five- to ten-membered heterocyclic group which may be substituted with a halogen atom,
or alternatively, when A is —CONR^{a}—, R^{a} and B may form together with an adjacent nitrogen atom a nitrogen-containing six-membered heterocyclic group which may have one or more substituents selected from a hydroxy group, a C₁₋₆ alkyl group, and a carbamoyl group;
ring Cy1 represents a benzene ring or a pyridine ring, each of which may have one or more substituents selected from a halogen atom and a C₁₋₆ alkyl group;
ring Cy2 represents a benzene ring or a pyridine ring which may have one or more substituents selected from a halogen atom and a C₁₋₆ alkoxy group;
ring Cy3 represents a five- or six-membered heterocyclic ring which may have one or more substituents selected from
   1) a C₁₋₆ alkyl group
   2) an oxo group and
   3) a halogen atom;
X represents —CH₂—, —CH₂—CH₂—, —CH(CH₃)—, —NH—, —CH(OH)—, —CH₂—O—, —C(CH₃)(OH)—, or —O—; and
ring Cy4 represents
   1) a benzene ring which may have one or more substituents selected from
      a) a halogen atom,
      b) a C₁₋₆ alkyl group which may be halogenated or hydroxylated,
      c) a C₁₋₆ alkoxy group
      d) an amino group which may be substituted with one or two C₁₋₆ alkyl groups, and
      e) a C₁₋₆ alkylsulfonyl group,
   2) a pyridine ring which may have one or more substituents selected from
      a) a C₁₋₆ alkyl group which may be halogenated, and
      b) a C₁₋₆ alkoxy,
   3) a pyridone ring which may have one or more substituents selected from
      a) a halogen atom, and
      b) a C₁₋₆ alkyl group which may be halogenated.

[10] The compound according to the above [2], wherein the chemical formula (I) is as follows:

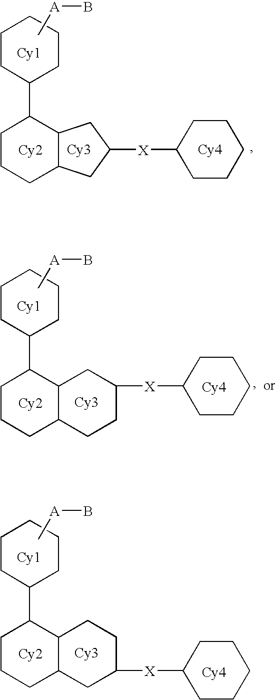

A represents —CONR^{a}— or —NR^{a}CO—;
R^{a} represents a hydrogen atom or a C₁₋₆ alkyl group a substituent;
B represents
   1) a hydrogen atom,
   2) a C₁₋₆ alkyl group which may have one or more substituents selected from
      a) a cyano group,
      b) a hydroxy group,
      c) a C₁₋₆ alkoxy group,
      d) a C₆₋₁₄ aryloxy group,
      e) a carbamoyl group, and
      f) an amino group which may be substituted with one or two substitutes selected from a C₁₋₆ alkyl group, a C₆₋₁₄ aryl group, a C₁₋₆ alkylcarbonyl group,
      g) a C₆₋₁₄ aryl group which may be substituted with an amino group which may be substituted with one or two C₁₋₆ alkyl groups,
      h) a five- or six-membered heterocyclic group which may be substituted with a substituent selected from a C₁₋₆ alkyl group and an oxo group,
      i) a C₁₋₆ alkylsulfanyl group,
      j) a C₁₋₆ alkylsulfinyl group, and
      k) a C₁₋₆ alkylsulfonyl group,
   3) a C₃₋₆ cycloalkyl group which may be substituted with a hydroxy group,
   4) a C₆₋₁₄ aryl group which may be substituted with a five- or six-membered heterocyclic group, or
   5) a five- to ten-membered heterocyclic group which may be substituted with a halogen atom,
or alternatively, when A is —CONR^{a}—, R^{a} and B may form together with an adjacent nitrogen atom a nitrogen-containing six-membered heterocyclic group which may have one or more substituents selected from a hydroxy group, a C₁₋₆ alkyl group, and a carbamoyl group;
ring Cy1 represents a benzene ring or a pyridine ring;
ring Cy2 represents a benzene ring or a pyridine ring which may have one or more substituents selected from a halogen atom and a C₁₋₆ alkoxy group;
ring Cy3 represents a five- or six-membered heterocyclic ring which may have one or more substituents selected from a C₁₋₆ alkyl group and an oxo group;
X represents a C₁₋₂ alkylene, —NH—, or —O—; and
ring Cy4 represents a benzene ring which may have one or more substituents selected from a halogen atom, a C₁₋₆ alkyl group which may be halogenated, and a C₁₋₆ alkoxy group.

[11] The compound according to the above [9], wherein the chemical formula (Ia) is the chemical formula (II)

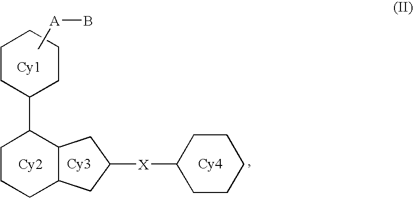

A is —CONR^{a},
R^{a} is a hydrogen atom, or a C₁₋₆ alkyl group,
B is
1) a hydrogen atom,
2) a C₁₋₆ alkyl group, which may have one or more substituents selected from
   a) a cyano group,
   b) a hydroxy group,
   c) a C₁₋₆ alkoxy group,
   d) a carbamoyl group,
   e) an amino group which may have one or two substituents selected from a C₁₋₆ alkyl group, a C₆₋₁₄ aryl group, and a C₁₋₆ alkyl-carbonyl group
   f) a C₁₋₆ alkylsulfinyl group,
   ring Cy1 is a benzene ring or a pyridine ring
   ring Cy2 is a benzene ring or a pyridine ring which may have one or more substituents selected from a halogen atom and a C₁₋₆ alkoxy group,
   ring Cy3 is a 5-membered heterocyclic ring which may have one or more substituents selected from C₁₋₆ alkyl group, and an oxo group
   X is C₁₋₂ alkylene, or —O—,
   ring Cy4 is
   a benzene ring which may have one or more substituents selected from
   1) a halogen atom,
   2) a C₁₋₆ alkyl group which may be halogenated,
   3) a C₁₋₆ alkoxy group, and
   4) a C₁₋₆ alkylsulfonyl group.

[12] The compound according to the above [10], wherein the chemical formula (I) is the chemical formula(II)

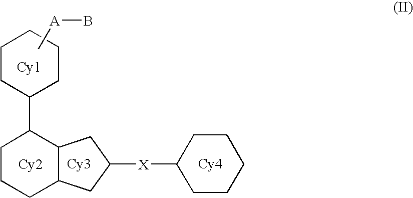

A is —CONR^{a},
R^{a} is a hydrogen atom, or a C₁₋₆ alkyl group,
B is
1) a hydrogen atom,
2) a C₁₋₆ alkyl group, which may have one or more substituents selected from
   a) a cyano group,
   b) a hydroxy group,
   c) a C₁₋₆ alkoxy group,
   d) a carbamoyl group,
   e) an amino group, which may have one or two substituents selected from a C₁₋₆ alkyl group, a C₆₋₁₄ aryl group, and a C₁₋₆ alkyl-carbonyl group
   f) a C₁₋₆ alkylsulfinyl group,
ring Cy1 is a benzene ring, or a pyridine ring,
ring Cy2 is a benzene ring, or a pyridine ring which may have one or more substituents selected from a halogen atom, and a C₁₋₆ alkoxy group,
ring Cy3 is a 5-membered heterocyclic ring which may have one or more substituents selected from
C₁₋₆ alkyl group, and an oxo group,
X is
C₁₋₂ alkylene, or —O—,
ring Cy4 is a benzene ring which may have one or more substituents selected from
   1) a halogen atom,
   2) a C₁₋₆ alkyl group which may be halogenated, and
   3) a C₁₋₆ alkoxy group.

[13] The compound according to the above [12], wherein the skeleton of the moiety represented by

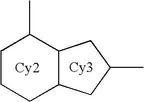

of the chemical formula (II) is a fused ring selected from

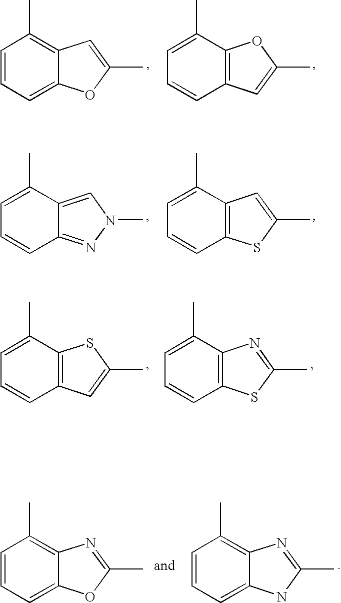

[14] The compound according to the above [12], the skeleton of the moiety represented by

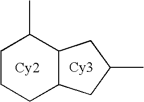

the chemical formula (II) is a fused ring selected from

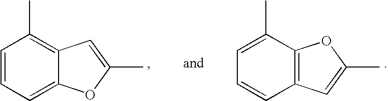

[15] The compound according to the above [12], the skeleton of the moiety represented by

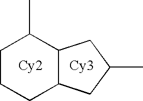

of the chemical formula (II) is a fused ring selected from

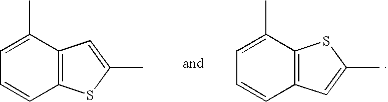

[16] The compound according to the above [10], wherein the chemical formula (I) is the chemical formula (III)

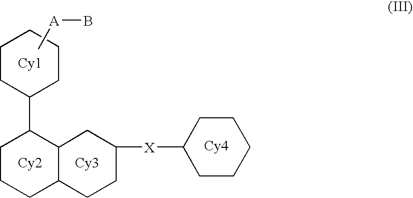

A is —CONR^{a},
R^{a} is a hydrogen atom,
B is
a C₁₋₆ alkyl group which may have one or more substituents selected from
   a) a cyano group, and
   b) a hydroxy group,
ring Cy1 is a benzene ring
the skeleton of the moiety represented by

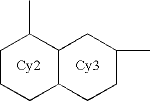

of the chemical formula (III) is a fused ring represented by

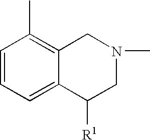

wherein R¹ is a hydrogen atom, or a C₁₋₆ alkyl group
X is C₁₋₂ alkylene,
ring Cy4 is a benzene ring which is substituted with C₁₋₆ alkyl group which may be halogenated.

[17] N-(2-hydroxyethyl)-3-[2-[3-(trifluoromethyl)benzyl]-1-benzofuran-4-yl]benzamide, or a salt there of.

[18] N-(2-hydroxyethyl)-3-[2-[3-(trifluoromethyl)benzyl]-1-benzofuran-4-yl]benzamide.

[19] 3-[2-(3-chloro-4-fluorobenzyl)-2H-indazol-4-yl]-N-(2-cyanoethyl)benzamide, or a salt thereof.

[20] N-(2-cyanoethyl)-3-{1-methyl-2-[3-(trifluoromethyl)benzyl]-1H-benzimidazol-4-yl}benzamide, or a salt thereof.

[21] N-(2-methoxyethyl)-3-[1-methyl-2-[3-(trifluoromethyl)phenoxy]-1H-benzimidazol-4-yl]benzamide, or a salt thereof.

[22] 3-[2-[[3-(trifluoromethyl)phenoxy]methyl]-1-benzothiophen-7-yl]benzamide, or a salt thereof.

[23] 3-[2-[[3-(trifluoromethyl)phenoxy]methyl]-1-benzothiophen-7-yl]benzamide.

[24] N-(2-hydroxyethyl)-3-[3-methyl-2-[3-(trifluoromethyl)benzyl]-1-benzothiophen-7-yl]benzamide, or a salt thereof.

5 [25] N-(2-hydroxyethyl)-3-[3-methyl-2-[3-(trifluoromethyl)benzyl]-1-benzothiophen-7-yl]benzamide.

[26] N-(2-hydroxyethyl)-2-{2-[3-(trifluoromethyl)benzyl]-1-benzothiophen-7-yl}pyridine-4-carboxamide, or a salt thereof.

[27] N-(2-amino-2-oxoethyl)-3-[4-fluoro-2-[3-(trifluoromethyl)benzyl]-1-benzothiophen-7-yl]benzamide, or a salt thereof.

[28] N-(2-amino-2-oxoethyl)-3-[4-fluoro-2-[3-(trifluoromethyl)benzyl]-1-benzothiophen-7-yl]benzamide.

[29] N-(2-amino-2-oxoethyl)-3-[2-[3-(trifluoromethyl)benzyl]-1,3-benzothiazol-4-yl]benzamide, or a salt thereof.

[30] 3-[2-(3-chloro-5-fluorobenzyl)-1-benzothiophen-7-yl]-N-[2-(1-methylethoxy)ethyl]benzamide, or a salt thereof.

[31] 3-[2-(3-chloro-5-fluorobenzyl)-1-benzothiophen-7-yl]-N-[2-(1-methylethoxy)ethyl]benzamide.

[32] A prodrug of the compound according to the above [1] or the above [2].

[33] A pharmaceutical agent comprising:

the compound according to the above [1] or the above [2] or the prodrug according to the above [32].

[34] The pharmaceutical agent according to the above [33] which is a GPR52 activating agent.

[35] The pharmaceutical agent according to the above [34] which is a preventive or therapeutic agent for schizophrenia.

[36] A GPR52 activating agent comprising a compound represented by formula (I₀):

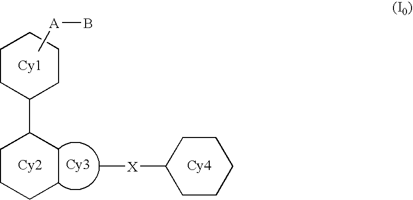

wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
   a halogen atom,
   a cyano group,
   a hydroxy group,
   a hydrocarbon-oxy group which may have one or more substituents,
   a chain hydrocarbon group which may have one or more substituents,
   a heterocyclic group which may have one or more substituents,
   an amino group which may have one or more substituents,
   an acyl group, and
   a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
ring Cy4 represents a six-membered aromatic ring which may have one or more substituents; with the proviso that
N-(3-(2-((4-chloro-2-methoxy-6-methylphenyl)amino)-1-methyl-1H-benzoimidazol-7-yl)phenyl)acetamide) is excluded;
or a salt thereof or a prodrug thereof.

[37] The GPR52 activating agent according to the above [36] which is a preventive or therapeutic agent for schizophrenia.
[38] A method of activating GPR52 comprising administrating an effective amount of a compound of formula (I₀):

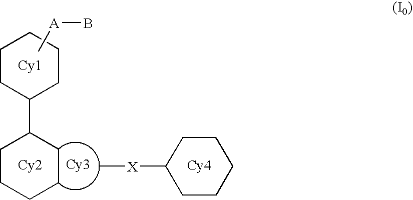

wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents; or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
   a halogen atom,
   a cyano group,
   a hydroxy group,
   a hydrocarbon-oxy group which may have one or more substituents,
   a chain hydrocarbon group which may have one or more substituents,
   a heterocyclic group which may have one or more substituents,
   an amino group which may have one or more substituents,
   an acyl group, and
   a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
ring Cy4 represents a six-membered aromatic ring which may have one or more substituents; with the proviso that
N-(3 -(2-((4-chloro-2-methoxy-6-methylphenyl)amino)-1-methyl-1H-benzoimidazol-7-yl)phenyl)acetamide) is excluded;
or a salt thereof or a prodrug thereof to the subject.

[39] A method of treating or preventing schizophrenia comprising administrating an effective amount of a compound of formula (I₀):

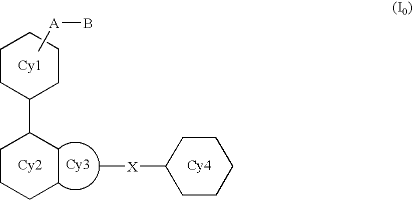

wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
   a halogen atom,
   a cyano group,
   a hydroxy group,
   a hydrocarbon-oxy group which may have one or more substituents,
   a chain hydrocarbon group which may have one or more substituents,
   a heterocyclic group which may have one or more substituents,
   an amino group which may have one or more substituents,
   an acyl group, and
   a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
ring Cy4 represents a six-membered aromatic ring which may have one or more substituents;
with the proviso that
N-(3-(2-((4-chloro-2-methoxy-6-methylphenyl)amino)-1-methyl-1H-benzoimidazol-7-yl)phenyl)acetamide) is excluded;
or a salt thereof or a prodrug thereof to the subject.

[40] Use of a compound represented by formula (I₀):

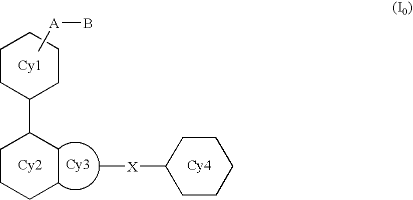

wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
   a halogen atom,
   a cyano group,
   a hydroxy group,
   a hydrocarbon-oxy group which may have one or more substituents,
   a chain hydrocarbon group which may have one or more substituents,
   a heterocyclic group which may have one or more substituents,
   an amino group which may have one or more substituents,
   an acyl group, and
   a carboxy group which may be esterified,
ring Cy3 represents a five- or six-membered ring which may have one or more substituents, X represents a C₁₋₂ alkylene which may be substituted with hydroxy, —Y—, Y—CH₂—, or —CH₂—Y—,
Y represents —O—, —NR^{b}, or
R^{b} represents a hydrogen atom or a substituent,
m represents an integer of 0 to 2,
ring Cy4 represents a six-membered aromatic ring which may have one or more substituents; with the proviso that
N-(3-(2-((4-chloro-2-methoxy-6-methylphenyl)amino)-1-methyl-1H-benzoimidazol-7-yl)phenyl)acetamide) is excluded;
or a salt thereof or a prodrug thereof in the manufacture of a GPR52 activating agent.

[41] Use of a compound represented by formula (I₀):

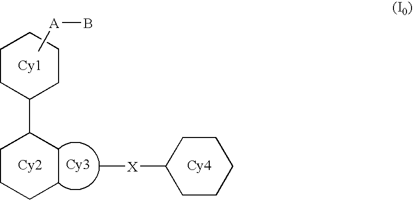

wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
   a halogen atom,
   a cyano group,
   a hydroxy group,
   a hydrocarbon-oxy group which may have one or more substituents,
   a chain hydrocarbon group which may have one or more substituents,
   a heterocyclic group which may have one or more substituents,
   an amino group which may have one or more substituents,
   an acyl group, and
   a carboxy group which may be esterified,
ring Cy3 represents a five- or six-membered ring which may have one or more substituents, X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
ring Cy4 represents a six-membered aromatic ring which may have one or more substituents; with the proviso that
N-(3-(2-((4-chloro-2-methoxy-6-methylphenyl)amino)-1-methyl-1H-benzoimidazol-7-yl)phenyl)acetamide) is excluded;
or a salt thereof or a prodrug thereof
in the manufacture of a preventive or therapeutic of schizophrenia.

[42] A compound represented by formula (I₀):

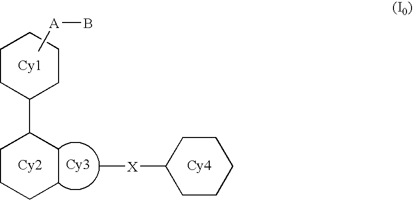

wherein
A represents —CONR^{a}— or —NR^{a}CO—;

R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
   a halogen atom,
   a cyano group,
   a hydroxy group,
   a hydrocarbon-oxy group which may have one or more substituents,
   a chain hydrocarbon group which may have one or more substituents,
   a heterocyclic group which may have one or more substituents,
   an amino group which may have one or more substituents,
   an acyl group, and
   a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
ring Cy4 represents a six-membered aromatic ring which may have one or more substituents;
with the proviso that
N-(3-(2-((4-chloro-2-methoxy-6-methylphenyl)amino)-1-methyl-1H-benzoimidazol-7-yl)phenyl)acetamide) is excluded;
or a salt thereof or a prodrug thereof
for use in activating GPR52.
[43] A compound represented by formula (I₀):

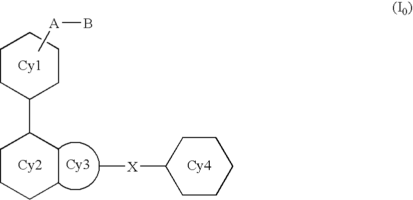

wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
   a halogen atom,
   a cyano group,
   a hydroxy group,
   a hydrocarbon-oxy group which may have one or more substituents,
   a chain hydrocarbon group which may have one or more substituents,
   a heterocyclic group which may have one or more substituents,
   an amino group which may have one or more substituents,
   an acyl group, and
   a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
ring Cy4 represents a six-membered aromatic ring which may have one or more substituents;
with the proviso that
N-(3-(2-((4-chloro-2-methoxy-6-methylphenyl)amino)-1-methyl-1H-benzoimidazol-7-yl)phenyl)acetamide) is excluded;
or a salt thereof or a prodrug thereof
for use in treating or preventing schizophrenia.

### Advantageous Effects of Invention

The compound of the present invention has an agonistic effect on GPR52 and is advantageously used as a preventive/therapeutic pharmaceutical agent for mental diseases such as schizophrenia.

### DESCRIPTION OF EMBODIMENTS

Hereinafter, the present invention will be described in detail.

Unless otherwise noted, the “halogen atoms” used herein include fluorine, chlorine, bromine, and iodine.

Unless otherwise noted, the expression “which may be halogenated” used herein means that one or more (e.g., one to three) halogen atoms may be provided as substituents.

Unless otherwise noted, the “carboxy (group) which may be esterified” used herein include carboxy, lower alkoxy-carbonyl which may be substituted, C₆₋₁₄ aryloxy-carbonyl which may be substituted, C₇₋₁₆ aralkyloxy-carbonyl which may be substituted, and silyloxy-carbonyl which may be substituted (e.g., TMS—O—CO—, TES—O—CO—, TBS—O—CO—, TIPS—O—CO—, and TBDPS—O—CO—).

Unless otherwise noted, for example, the “lower alkoxy-carbonyl (group)” used herein may be any of methoxycarbonyl, ethoxycarbonyl, propoxycarbonyl, and tert-butoxycarbonyl.

Unless otherwise noted, for example, the “C₆₋₁₄ aryloxy-carbonyl (group)” used herein may be a phenoxycarbonyl.

Unless otherwise noted, for example, the “C₇₋₁₆ aralkyloxy-carbonyl (group)” used herein may be any of benzyloxycarbonyl and phenethyloxycarbonyl.

Unless otherwise noted, for example, the “lower alkyl (group)” used herein may be C₁₋₆ alkyl (group).

Unless otherwise noted, for example, the “C₁₋₆ alkyl (group)” used herein may be any of methyl, ethyl, propyl, isopropyl, butyl, isobutyl, sec-butyl, tert-butyl, pentyl, isopentyl, neopentyl, and hexyl.

Unless otherwise noted, the “C₁₋₆ alkyl (group) which may be halogenated” used herein means C₁₋₆ alkyl (group) which may be substituted with a halogen atom and the example thereof may be trifluoromethyl.

Unless otherwise noted, for example, the “lower alkenyl (group)” used herein may be C₂₋₆ alkenyl (group).

Unless otherwise noted, for example, the “C₂₋₆ alkenyl (group)” used herein may be any of vinyl, 1-propen-1-yl, 2-propen-1-yl, isopropenyl, 2-buten-1-yl, 4-penten-1-yl, and 5-hexen-1-yl.

Unless otherwise noted, the “lower alkynyl (group)” used herein may be a C₂₋₆ alkynyl group. Examples of the “C₂₋₆ alkyl (group)” used herein include ethynyl, 1-propyn-1-yl, 2-propyn-1-yl, 4-pentyn-1-yl, and, 5-hexyn-1-yl.

Unless otherwise noted, for example, “C₃₋₈ cycloalkyl (group)” used herein may be any of cyclopropyl, cyclobutyl, cyclopentyl, and cyclohexyl.

Unless otherwise noted, for example, the “C₆₋₁₄ aryl (group)” used herein may be any of phenyl, 1-naphtyl, 2-naphtyl, 2-biphenyl, 3-biphenyl, 4-biphenyl, and 2-anthryl.

Unless otherwise noted, for example, the “C₇₋₁₆ aralkyl (group)” used herein may be any of benzyl, phenethyl, diphenylmethyl, 1-naphtylmethyl, 2-naphtylmethyl, 2,2-diphenylethyl, 3-phenylpropyl, 4-phenylbutyl, 5-phenylpentyl, 2-biphenylmethyl, 3-biphenylmethyl, and 4-biphenylmethyl.

Unless otherwise noted, for example, the “C₆₋₁₄ aryl-C₂₋₆ alkenyl (group)” used herein may be styryl.

Unless otherwise noted, examples of the “heterocyclic group” (and heterocyclic ring portions in the substituents) used herein include: 3- to 14-membered (monocyclic, bicyclic, or tricyclic) heterocyclic groups with one to five of one to three kinds of heteroatoms selected from a nitrogen atom, a sulfur atom, and an oxygen atom in addition to carbon atoms. Examples of such heterocyclic groups include aromatic heterocyclic group such as pyrrolyl (e.g., 1- pyrrolyl, 2-pyrrolyl, 3-pyrrolyl), furyl (e.g., 2-furyl, 3-furyl), thienyl (e.g., 2-thienyl, 3-thienyl), pyrazolyl (e.g., 1-pyrazolyl, 3-pyrazolyl, 4-pyrazolyl), imidazolyl (e.g., 1-imidazolyl, 2-imidazolyl, 4-imidazolyl), isoxazolyl (e.g., 3-isoxazolyl, 4-isoxazolyl, 5-isoxazolyl), oxazolyl (e.g., 2-oxazolyl, 4-oxazolyl, 5-oxazolyl), isothiazolyl (e.g., 3-isothiazolyl, 4-isothiazolyl, 5-isothiazolyl), thiazolyl (e.g., 2-thiazolyl, 4-thiazolyl, 5-thiazolyl), triazolyl (1,2,3-triazol-4-yl, 1,2,4-triazol-3-yl), oxadiazolyl (1,2,4-oxadiazol-3-yl, 1,2,4-oxadiazol-5-yl), thiadiazolyl (1,2,4-thiadiazol-3-yl, 1,2,4-thiadiazol-5-yl), tetrazolyl, pyridyl (e.g., 2-pyridyl, 3-pyridyl, 4-pyridyl), pyridazinyl (e.g., 3-pyridazinyl, 4-pyridazinyl), pyrimidinyl (e.g., 2-pyrimidinyl, 4-pyrimidinyl, 5-pyrimidinyl), pyrazinyl, isoindolyl (e.g., 1-isoindolyl, 2-isoindolyl, 3-isoindolyl, 4-isoindolyl, 5-isoindolyl, 6-isoindolyl, 7-isoindolyl), indolyl (e.g., 1-indolyl, 2-indolyl, 3-indolyl, 4-indolyl, 5-indolyl, 6-indolyl, 7-indolyl), benzo[b]furanyl (e.g., 2-benzo[b]furanyl, 3-benzo[b]furanyl, 4-benzo[b]furanyl, 5-benzo[b]furanyl, 6-benzo[b]furanyl, 7-benzo[b]furanyl), benzo[c]furanyl (e.g., 1-benzo[c]furanyl, 4-benzo[c]furanyl, 5-benzo[c]furanyl), benzo[b]thienyl, (e.g., 2-benzo[b]thienyl, 3-benzo[b]thienyl, 4-benzo[b]thienyl, 5-benzo[b]thienyl, 6-benzo[b]thienyl, 7-benzo[b]thienyl), benzo[c]thienyl (e.g., 1-benzo[c]thienyl, 4-benzo[c]thienyl, 5-benzo[c]thienyl), indazolyl (e.g., 1-indazolyl, 2-indazolyl, 3-indazolyl, 4-indazolyl, 5-indazolyl, 6-indazolyl, 7-indazolyl), benzoimidazolyl (e.g., 1-benzoimidazolyl, 2-benzoimidazolyl, 4-benzoimidazolyl, 5-benzoimidazolyl), 1,2-benzoisoxazolyl (e.g., 1,2-benzoisoxazol-3-yl, 1,2-benzoisoxazol-4-yl, 1,2-benzoisoxazol-5-yl, 1,2-benzoisoxazol-6-yl, 1,2-benzoisoxazol-7-yl), benzooxazolyl (e.g., 2-benzooxazolyl, 4-benzooxazolyl, 5-benzooxazolyl, 6-benzooxazolyl, 7-benzooxazolyl), 1,2-benzoisothiazolyl (e.g., 1,2-benzoisothiazol-3-yl, 1,2-benzoisothiazol-4-yl, 1,2-benzoisothiazol-5-yl, 1,2-benzoisothiazol-6-yl, 1,2-benzoisothiazol-7-yl), benzothiazolyl (e.g., 2-benzothiazolyl, 4-benzothiazolyl, 5-benzothiazolyl, 6-benzothiazolyl, 7-benzothiazolyl), isoquinolyl (e.g., 1-isoquinolyl, 3-isoquinolyl, 4-isoquinolyl, 5-isoquinolyl), quinolyl (e.g., 2-quinolyl, 3-quinolyl, 4-quinolyl, 5-quinolyl, 8-quinolyl), cinnolinyl (e.g., 3-cinnolinyl, 4-cinnolinyl, 5-cinnolinyl, 6-cinnolinyl, 7-cinnolinyl, 8-cinnolinyl), phthalazinyl (e.g., 1-phthalazinyl, 4-phthalazinyl, 5-phthalazinyl, 6-phthalazinyl, 7-phthalazinyl, 8-phthalazinyl), quinazolinyl (e.g., 2-quinazolinyl, 4-quinazolinyl, 5-quinazolinyl, 6-quinazolinyl, 7-quinazolinyl, 8-quinazolinyl), quinoxalinyl (e.g., 2-quinoxalinyl, 3-quinoxalinyl, 5-quinoxalinyl, 6-quinoxalinyl, 7-quinoxalinyl, 8-quinoxalinyl), pyrazolo[1,5-a]pyridyl(pyrazolo[1,5-a]pyridin-2-yl, pyrazolo[1,5-a]pyridin-3-yl, pyrazolo[1,5-a]pyridin-4-yl, pyrazolo[1,5-a]pyridin-5-yl, pyrazolo[1,5-a]pyridin-6-yl, pyrazolo[1,5-a]pyridin-7-yl), and imidazo[1,2-a]pyridyl (imidazo[1,2-a]pyridin-2-yl, imidazo[1,2-a]pyridin-3-yl, imidazo[1,2-a]pyridin-5-yl, imidazo[1,2-a]pyridin-6-yl, imidazo[1,2-a]pyridin-7-yl, imidazo[1,2-a]pyridin-8-yl); and nonaromatic heterocyclic groups such as tetrahydrofuryl, oxazolidinyl, imidazolinyl (e.g., 1-imidazolinyl, 2-imidazolinyl, 4-imidazolinyl), aziridinyl (e.g., 1-aziridinyl, 2-aziridinyl), azetidinyl (e.g., 1-azetidinyl, 2-azetidinyl), pyrrolidinyl (e.g., 1-pyrrolidinyl, 2-pyrrolidinyl, 3-pyrrolidinyl), piperidinyl (e.g., 1-piperidinyl, 2-piperidinyl, 3-piperidinyl), azepanyl (e.g., 1-azepanyl, 2-azepanyl, 3-azepanyl, 4-azepanyl), azocanyl (e.g., 1-azocanyl, 2-azocanyl, 3-azocanyl, 4-azocanyl), piperazinyl (e.g., 1,4-piperazin-1-yl, 1,4-piperazin-2-yl), diazepane-yl (e.g., 1,4-diazepan-1-yl, 1,4-diazepan-2-yl, 1,4-diazepan-5-yl, 1,4-diazepan-6-yl), diazocanyl(1,4-diazocan-1-yl, 1,4-diazocan-2-yl, 1,4-diazocan-5-yl, 1,4-diazocan-6-yl, 1,5-diazocan-1-yl, 1,5-diazocan-2-yl, 1,5-diazocan-3-yl), tetrahydropyranyl (e.g., tetrahydropyran-4-yl), morpholinyl (e.g., 4-morpholinyl), thiomorpholinyl (e.g., 4-thiomorpholinyl), and 2-oxazolidinyl; heterocyclic groups obtained by partially hydrogenating the above aromatic heterocyclic groups (e.g., heterocyclic groups such as indolyl, and dihydroquinolyl); and heterocyclic groups obtained by partially dehydrogenating the above nonaromatic heterocyclic groups (e.g., dihydrofuranyl).

Unless otherwise noted, for example, the examples of “nitrogen-containing heterocyclic group” used herein include the same nitrogen-containing heterocyclic groups among the above “heterocyclic group”

Unless otherwise noted, for example, the “lower alkoxy (group)” used herein may be C₁₋₆ alkoxy.

Unless otherwise noted, for example, the “C₁₋₆ alkoxy (group)” used herein may be any of methoxy, ethoxy, propoxy, isopropoxy, butoxy, isobutoxy, sec-butoxy, pentyloxy, and hexyloxy.

Unless otherwise noted, for example, the “C₃₋₈ cycloalkyloxy (group)” used herein may be any of cyclopropyloxy, cyclobutyloxy, cyclopentyloxy, and cyclohexyloxy.

Unless otherwise noted, for example, the “C₆₋₁₄ aryloxy (group)” used herein may be any of phenyloxy, 1-naphthyloxy, and 2-naphthyloxy.

Unless otherwise noted, for example, the “C₇₋₁₆ aralkyloxy (group)” may be any of benzyloxy and phenethyloxy.

Unless otherwise noted, for example, the “lower alkyl-carbonyloxy (group)” used herein may be C₁₋₆ alkyl-carbonyloxy.

Unless otherwise noted, for example, the “C₁₋₆ alkyl-carbonyloxy (group)” used herein may be acetoxy and propionyloxy.

Unless otherwise noted, for example, the “lower alkoxy-carbonyloxy (group)” used herein may be C₁₋₆ alkoxy-carbonyloxy (group).

Unless otherwise noted, for example, the “C₁₋₆ alkoxy-carbonyloxy (group)” used herein may be any of methoxycarbonyloxy, ethoxycarbonyloxy, propoxycarbonyloxy, and butoxycarbonyloxy.

Unless otherwise noted, for example, the “mono-lower alkyl-carbamoyloxy (group)” used herein may be mono-C₁₋₆ alkyl-carbamoyloxy (group).

Unless otherwise noted, for example, the “mono-C₁₋₆ alkyl-carbamoyloxy (group)” used herein may be any of methylcarbamoyloxy and ethylcarbamoyloxy.

Unless otherwise noted, for example, the “di-lower alkyl-carbamoyloxy (group)” used herein may be di-C₁₋₆ alkyl-carbamoyloxy (group).

Unless otherwise noted, for example, the “di-C₁₋₆ alkyl-carbamoyloxy (group)” used herein may be any of dimethylcarbamoyloxy and diethylcarbamoyloxy.

Unless otherwise noted, for example, the “C₆₋₁₄ aryl-carbonyloxy (group)” used herein may be any of benzoyloxy and naphthylcarbonyloxy.

Unless otherwise noted, for example, the “mono- or di-C₆₋₁₄ aryl-carbamoyloxy (group)” used herein may be phenylcarbamoyloxy and naphthylcarbamoyloxy.

Unless otherwise noted, for example, the heterocyclic moiety of the “heterocyclic oxy (group)” used herein may be the same “heterocyclic group” as any of those described above. Specifically, examples of the “heterocyclic oxy (group)” include 5- to 14-membered heterocyclic-oxy (group) that contains one to five of one to three kinds of heteroatoms selected from a nitrogen atom, a sulfur atom, and an oxygen atom in addition to carbon atoms.

Unless otherwise noted, for example, the aromatic heterocyclic moiety of the “aromatic heterocyclic oxy (group)” used herein may be the same “aromatic heterocyclic group” as one provided as an example of the aforementioned “heterocyclic group”. Specifically, examples of the “aromatic heterocyclic oxy (group)” include 3- to 14-membered aromatic heterocyclic-oxy containing one to five of one to three kinds of heteroatoms selected from a nitrogen atom, a sulfur atom, and an oxygen atom in addition to carbon atoms.

Unless otherwise noted, for example, the “lower alkylthio (group)” used herein may be C₁₋₆ alkylthio (group).

Unless otherwise noted, for example, the “C₁₋₆ alkylthio (group)” used herein may be any of methylthio, ethylthio, propylthio, isopropylthio, butylthio, sec-butylthio, and tert-butylthio.

Unless otherwise noted, for example, the “C₃₋₈ cycloalkylthio (group)” used herein may be any of cyclopropylthio, cyclobutylthio, cyclopentylthio, and cyclohexylthio.

Unless otherwise noted, for example, the “C₆₋₁₄ arylthio (group)” used herein may be any of phenylthio, 1-napthtylthio, and 2-napthtylthio.

Unless otherwise noted, for example, the “C₇₋₁₆ aralkylthio (group)” used herein may be benzylthio and phenethylthio.

Unless otherwise noted, for example, the heterocyclic ring moiety of the “heterocyclic thio (group)” may be the same “heterocyclic group” as one described above. Specifically, the “heterocyclic thio (group)” may be 5- to 14-membered heterocyclic thio (group) containing one to five of one to three kinds heteroatoms selected from a nitrogen atom, a sulfur atom, and an oxygen atom in addition to carbon atoms.

Unless otherwise noted, for example, the “lower alkylcarbonyl (group)” used herein may be C₁₋₆ alkyl-carbonyl. Unless otherwise noted, for example, the “C₁₋₆ alkyl-carbonyl (group)” used herein may be any of acetyl, propionyloxy, and pivaloyl.

Unless otherwise noted, for example, the “C₃₋₈ cycloalkylcarbonyl (group)” used herein may be cyclopropylcarbonyl, cyclopentylcarbonyl, and cyclohexylcarbonyl.

Unless otherwise noted, for example, the “C₆₋₁₄ aryl-carbonyl (group)” used herein may be any of benzoyl, 1-naphthoyl, and 2-naphthoyl.

Unless otherwise noted, for example, the “C₇₋₁₆ aralkyl-carbonyl (group)” used herein may be any of phenylacetyl and 3-phenylpropionyloxy.

Unless otherwise noted, for example, the heterocyclic ring moiety of the “heterocyclic carbonyl (group)” may be the same “heterocyclic group” as one described above. Specifically, it may be 3- to 14-membered heterocyclic carbonyl (group) containing one to five of one to three kind heteroatoms selected from a nitrogen atom, a sulfur atom, and an oxygen atom in addition to carbon atoms. More specifically, for example, such a heterocyclic ring moiety include picolinoyl, nicotinoyl, iso-nicotinoyl, 2-thenoyl, 3-thenoyl, 2-furoyl, 3-furoyl, 1-morpholinylcarbonyl, 4-thiomorpholinylcarbonyl, aziridin-1-yl-carbonyl, aziridin-2-yl-carbonyl, azetidin-1-yl-carbonyl, azetidin-2-yl-carbonyl, pyrrolidin-1-yl-carbonyl, pyrrolidin-2-yl-carbonyl, pyrrolidin-3-yl-carbonyl, piperidin-1-yl-carbonyl, piperidin-2-yl-carbonyl, piperidin-3-yl-carbonyl, azepan-1-yl-carbonyl, azepan-2-yl-carbonyl, azepan-3-yl-carbonyl, azepan-4-yl-carbonyl, azocan-1-yl-carbonyl, azocan-2-yl-carbonyl, azocan-3-yl-carbonyl, azocan-4-yl-carbonyl, 1,4-piperazin-1-yl-carbonyl, 1,4-piperazin-2-yl-carbonyl, 1,4-diazepan-1-yl-carbonyl, 1,4-diazepan-2-yl-carbonyl, 1,4-diazepan-5-yl-carbonyl, 1,4-diazepan-6-yl-carbonyl, 1,4-diazocan-1-yl-carbonyl, 1,4-diazocan-2-yl-carbonyl, 1,4-diazocan-5-yl-carbonyl, 1,4-diazocan-6-yl-carbonyl, 1,5-diazocan-1-yl-carbonyl, 1,5-diazocan-2-yl-carbonyl, and 1,5-diazocan-3-yl-carbonyl.

Unless otherwise noted, for example, the “lower alkylsulfonyl (group)” used herein may be C₁₋₆ alkylsulfonyl (group).

Unless otherwise noted, for example, the “C₁₋₆ alkylsulfonyl (group)” used herein may be any of methylsulfonyl and ethylsulfonyl.

Unless otherwise noted, for example, the “C₃₋₈ cycloalkylsulfonyl (group)” used herein may be any of cyclopropylsulfonyl, cyclobutylsulfonyl, cyclopentylsulfonyl, and cyclohexylsulfonyl.

Unless otherwise noted, for example, the “C₆₋₁₄ arylsulfonyl (group)” used herein may be any of phenylsulfonyl, 1-naphthylsulfonyl, and 2-naphthylsulfonyl.

Unless otherwise noted, for example, the heterocyclic ring moiety of the “heterocyclic sulfonyl (group)” may be the same “heterocyclic group” as one described above. Specifically, “heterocyclic sulfonyl (group)” may be 5- to 14-membered heterocyclic sulfonyl (group) containing one to five of one to three kinds of heteroatoms selected from a nitrogen atom, a sulfur atom, and an oxygen atom and hetero atoms in addition to carbon atoms.

Unless otherwise noted, for example, the “lower alkylsulfinyl (group)” used herein may be C₁₋₆ alkylsulfinyl (group). Unless otherwise noted, for example, the “C₁₋₆ alkylsulfinyl (group)” used herein may be any of methylsulfinyl and ethylsulfinyl.

Unless otherwise noted, for example, the “C₃₋₈ cycloalkylsulfinyl (group)” used herein may be any of cyclopropylsulfinyl, cyclobutylsulfinyl, cyclopentylsulfinyl, and cyclohexylsulfinyl.

Unless otherwise noted, for example, the “C₆₋₁₄ arylsulfinyl (group)” used herein may be any of phenylsulfinyl, 1-naphthylsulfinyl, and 2-naphthylsulfinyl.

Unless otherwise noted, for example, the heterocyclic ring moiety of the “heterocyclic sulfinyl (group)” may be the same “heterocyclic group” as one described above. Specifically, for example, “heterocyclic sulfinyl (group)” may be 5- to 14-membered heterocyclic sulfinyl (group) containing one to five of one to three kinds of heteroatoms selected from a nitrogen atom, a sulfur atom, and an oxygen atom in addition to carbon atoms.

Unless otherwise noted, for example, the “lower alkyl-carbamoyl (group)” used herein may be C₁₋₆ alkyl-carbamoyl.

Unless otherwise noted, for example, the “C₁₋₆ alkyl-carbamoyl (group)” used herein may be any of methylcarbamoyl, ethylcarbamoyl, and propylcarbamoyl.

Unless otherwise noted, for example, the “mono- or di-lower alkylamino (group)” used herein may be mono- or di-C₁₋₆ alkylamino (group).

Unless otherwise noted, for example, the “mono- or di-C₁₋₆ alkylamino (group)” used herein may be any of methylamino, ethylamino, propylamino, dimethylamino, and diethylamino.

Unless otherwise noted, for example, the “lower alkyl-carbonylamino (group)” used herein may be C₁₋₆ alkyl-carbonylamino.

Unless otherwise noted, for example, the “C₁₋₆ alkyl-carbonylamino (group)” used herein may be any of acetylamino, propionylamino, and pivaloylamino.

Unless otherwise noted, for example, the “heterocyclic ring (group)” of the “heterocyclic amino (group)” used herein may be the same “heterocyclic ring group” as one described above. For example, the “heterocyclic amino (group)” used herein may be 2-pyridyl-amino.

Unless otherwise noted, for example, the “heterocyclic carbonyl” of the “heterocyclic carbonylamino (group)” used herein may be the same “heterocyclic carbonyl” as one described above. For example, the “heterocyclic carbonylamino (group)” used herein may be pyridyl-carbonylamino.

Unless otherwise noted, for example, the “heterocyclic ring (group)” of the “heterocyclic ring-oxycarbonylamino (group)” used herein may be in the same “heterocyclic ring group” as one described above. For example, the “heterocyclic ring-oxycarbonylamino (group)” used herein may be 2-pyridyl-oxycarbonylamino.

Unless otherwise noted, for example, the “heterocyclic ring (group)” of the “heterocyclic-sulfonylamino (group)” used herein may be the same “heterocyclic group” as one described above. For example, the “heterocyclic sulfonylamino (group)” may be 2-pyridyl-sulfonylamino.

Unless otherwise noted, for example, the “lower alkoxy-carbonylamino (group)” used herein may be C₁₋₆ alkoxy-carbonylamino (group).

Unless otherwise noted, for example, the “C₁₋₆ alkoxy-carbonylamino (group)” used herein may be any of methoxycarbonylamino, ethoxycarbonylamino, propoxycarbonylamino, and butoxycarbonylamino.

Unless otherwise noted, for example, the “lower alkylsulfonylamino (group)” used herein may be C₁₋₆ alkylsulfonylamino (group).

Unless otherwise noted, for example, the “C₁₋₆ alkylsulfonylamino (group)” used herein may be any of methylsulfonylamino and ethylsulfonylamino.

Unless otherwise noted, for example, the “mono- or di-C₃₋₈ cycloalkylamino (group)” used herein may be any of cyclopropylamino, cyclopentylamino, and cyclohexylamino.

Unless otherwise noted, for example, the “C₃₋₈ cycloalkyl-carbonylamino (group)” used herein may be any of cyclopropyl-carbonylamino, cyclopentyl-carbonylamino, and cyclohexyl-carbonylamino.

Unless otherwise noted, for example, the “C₃₋₈ cycloalkoxy-carbonylamino (group)” used herein may be any of cyclopropoxycarbonylamino, cyclopentyloxycarbonylamino, and cyclohexyloxycarbonylamino.

Unless otherwise noted, for example, the “C₃₋₈ cycloalkyl-sulfonylamino (group)” used herein may be any of cyclopropylsulfonylamino, cyclopentylsulfonylamino, and cyclohexylsulfonylamino.

Unless otherwise noted, for example, the “mono- or di-C₆₋₁₄ arylamino (group)” used herein may be any of phenylamino and diphenylamino.

Unless otherwise noted, for example, the “mono- or di-C₇₋₁₆ aralkylamino (group)” used herein may be benzylamino.

Unless otherwise noted, for example, “C₆₋₁₄ aryl-carbonylamino” may be benzoylamino and naphthoylamino.

Unless otherwise noted, for example, the “C₆₋₁₄ arylsulfonylamino” may be phenylsulfonylamino, 2-naphthylsulfonylamino, and 1-naphthylsulfonylamino.

Hereinafter, symbols in the above formulae (formula (I₀), formula (Iₐ), and formula (I)) will be described.

In the above formulae, A represents —CONR^{a}— or —NR^{a}CO—. R^{a} represents a hydrogen atom or a substituent. The substituent represented by R^{a} may be a substituent selected from the following substituents listed in Substituent Group A.

### <Substituent Group A>

(1) Halogen atom;
(2) Nitro;
(3) Cyano;
(4) Carboxy which may be esterified;
(5) Lower alkyl which may be substituted;
(6) Lower alkenyl which may be substituted;
(7) Lower alkynyl which may be substituted;
(8) C₃₋₈ cycloalkyl which may be substituted;
(9) C₆₋₁₄ aryl which may be substituted;
(10) C₇₋₁₆ aralkyl which may be substituted;
(11) C₆₋₁₄ aryl-C₂₋₆ alkenyl which may be substituted;
(12) Heterocyclic group which may be substituted;
(13) Hydroxy;
(14) Lower alkoxy which may be substituted;
(15) C₃₋₈ cycloalkoxy which may be substituted;
(16) C₆₋₁₄ aryloxy which may be substituted;
(17) C₇₋₁₆ aralkyloxy which may be substituted;
(18) Lower alkyl-carbonyloxy which may be substituted;
(19) Lower alkoxy-carbonyloxy which may be substituted;
(20) Mono-lower alkyl-carbamoyloxy which may be substituted;
(21) Di-lower alkyl-carbamoyloxy which may be substituted;
(22) C₆₋₁₄ aryl-carbonyloxy which may be substituted;
(23) Mono- or di-C₆₋₁₄ aryl-carbamoyloxy which may be substituted;
(24) Heterocyclic oxy which may be substituted (e.g., aromatic heterocyclic oxy which may be substituted);
(25) Mercapto
(26) Lower alkylthio which may be substituted;
(27) C₃₋₈ cycloalkylthio which may be substituted;
(28) C₆₋₁₄ arylthio which may be substituted;
(29) C₇₋₁₆ aralkylthio which may be substituted;
(30) Heterocyclic thio which may be substituted;
(31) Formyl;
32) Lower alkyl-carbonyl which may be substituted;
(33) C₃₋₈ cycloalkyl-carbonyl which may be substituted;
(34) C₆₋₁₄ aryl-carbonyl which may be substituted;
(35) C₇₋₁₆ aralkyl-carbonyl which may be substituted;
(36) Heterocyclic-carbonyl which may be substituted;
(37) Lower alkylsulfonyl which may be substituted;
(38) C₃₋₈ cycloalkylsulfonyl which may be substituted;
(39) C₆₋₁₄ arylsulfonyl which may be substituted;
(40) Heterocyclic sulfonyl which may be substituted;
(41) Lower alkylsulfinyl which may be substituted;
(42) C₃₋₈ cycloalkylsulfinyl which may be substituted;
(43) C₆₋₁₄ arylsulfinyl which may be substituted;
(44) Heterocyclic sulfinyl which may be substituted;
(45) Sulfo;
(46) Sulfamoyl;
(47) Sulfinamoyl;
(48) Sulfenamoyl;
(49) Thiocarbamoyl;
(50) Carbamoyl group which may be substituted (e.g., lower alkyl carbamoyl which may be substituted);

(1) Amino group which may be substituted (e.g., amino, mono- or di-lower alkylamino which may be substituted, mono- or di-C₃₋₈ cycloalkylamino which may be substituted, mono- or di-C₆₋₁₄ arylamino which may be substituted; mono- or di-C₇₋₁₆ aralkylamino which may be substituted; heterocyclic amino which may be substituted, C₆₋₁₄ aryl-carbonylamino which may be substituted, formylamino, lower alkyl-carbonylamino which may be substituted, C₃₋₈ cycloalkyl-carbonylamino which may be substituted, heterocyclic-carbonylamino which may be substituted, lower alkoxy-carbonyl amino which may be substituted, C₃₋₈ cycloalkoxy-carbonylamino which may be substituted, heterocyclic ring-oxycarbonylamino which may be substituted, carbamoylamino group which may have one or more substituents, lower alkylsulfonylamino which may be substituted, C₃₋₈ cycloalkyl-sulfonylamino which may be substituted, heterocyclic-sulfonyl amino which may be substituted, and C₆₋₁₄ arylsulfonylamino which may be substituted).

Any of substitutes used for the aforementioned
“lower alkoxy-carbonyl which may be substituted”,
“lower alkyl which may be substituted”,
“lower alkenyl which may be substituted”,
“lower alkynyl which may be substituted”,
“lower alkoxy which may be substituted”,
“lower alkyl-carbonyloxy which may be substituted”,
“lower alkoxy-carbonyloxy which may be substituted”,
“mono-lower alkyl-carbamoyloxy which may be substituted”,
“di-lower alkyl-carbamoyloxy which may be substituted”,
“lower alkylthio which may be substituted”,
“lower alkyl-carbonyl which may be substituted”,
“lower alkylsulfonyl which may be substituted”,
“lower alkylsulfinyl which may be substituted”,
“mono- or di-lower alkylamino which may be substituted”,
“lower alkyl-carbonylamino which may be substituted”,
“lower alkoxy-carbonyl amino which may be substituted”, and
“lower alkylsulfonylamino which may be substituted”) may be selected from substituents listed in Substituent Group B below. In each case, the number of the substituents may be 1 to 5 a maximum substitutable number, preferably 1 to 3, more preferably 1.

### <Substituent Group B>

Halogen atom;
Hydroxy;
Nitro;
Cyano;

C₆₋₁₄ aryl, which may be substituted with a halogen atom, hydroxy, cyano, amino, C₁₋₆ alkyl which may be halogenated, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl, or the like);

C₆₋₁₄ aryloxy, which may be substituted with a halogen atom, hydroxy, cyano, amino, C₁₋₆ alkyl which may be halogenated, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl, or the like);

C₇₋₁₆ aralkyloxy, which may be substituted with a halogen atom, hydroxy, cyano, amino, C₁₋₆ alkyl which may be halogenated, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl, or the like);

Any of 5- to 10-membered mono- or di-heterocyclic groups each containing one to four of one or two kinds heteroatoms selected from a nitrogen atom, a sulfur atom, and an oxygen atom in addition to carbon atoms (e.g., furyl, pyridyl, thienyl, pyrrolidino, 1-piperidyl, 4-piperidyl, piperazinyl, 1-morpholinyl, 4-thiomorpholinyl, azepan-1-yl, azocan-1-yl, azonan-1-yl, 3,4-dihydroisoquinolin-2-yl, and so on) (the heterocyclic group may be substituted with a halogen atom, hydroxy, cyano, amino, C₁₋₆ alkyl which may be halogenated, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl, or the like);

Amino group which may be substituted (e.g., an amino group which may be substituted with one or two substituent selected from a group consisting of C₁₋₆ alkyl, C₂₋₆ alkenyl, C₆₋₁₄ aryl, C₇₋₁₆ aralkyl, a heterocyclic group, and heterocyclic ring-lower alkyl(each of the C₁₋₆ alkyl, C₂₋₆ alkenyl, C₆₋₁₄ aryl, C₇₋₁₆ aralkyl, heterocyclic group, and heterocyclic ring-lower alkyl may be substituted with a halogen atom, hydroxy, cyano, amino, C₁₋₆ alkyl which may be halogenated(but not any substituent of alkyl and alkenyl), mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxycarbonyl, C₃₋₈ cycloalkoxy-carbonyl, C₆₋₁₄ aryloxy-carbonyl, C₇₋₁₆ aralkyloxy-carbonyl, C₁₋₆ alkylthio, C₃₋₈ cycloalkylthio, C₁₋₆ alkylsulfinyl, C₃₋₈ cycloalkylsulfinyl, C₁₋₆ alkylsulfonyl, C₃₋₈ cycloalkylsulfonyl, carbamoyl, thiocarbamoyl, mono- or di-C₁₋₆ alkylcarbamoyl, mono- or di-C₆₋₁₄ arylcarbamoyl, or the like). Here, the “heterocyclic ring” and the “heterocyclic ring” of the “heterocyclic ring-lower alkyl” may be the same “heterocyclic group” as one described above);

C₃₋₈ cycloalkyl; C₁₋₆ alkoxy, which may be substituted with halogen atom, hydroxy, amino, mono- or di-C₁₋₆ alkylamino, mono- or di-C₆₋₁₄ arylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl, or the like);

Formyl;
C₁₋₆ alkyl-carbonyl (e.g., acetyl);
C₃₋₈ cycloalkyl-carbonyl;
C₆₋₁₄ aryl-carbonyl;
C₇₋₁₆ aralkyl-carbonyl;
C₁₋₆ alkoxycarbonyl;
C₆₋₁₄ aryloxy-carbonyl;
C₇₋₁₆ aralkyloxy-carbonyl;
C₁₋₆ alkylthio;
C₁₋₆ alkylsulfinyl;
C₁₋₆ alkylsulfonyl;
Carbamoyl;
Thiocarbamoyl;
Mono-C₁₋₆ alkyl-carbamoyl (e.g., methylcarbamoyl or ethylcarbamoyl);
Di-C₁₋₆ alkyl-carbamoyl (e.g., dimethylcarbamoyl, Diethylcarbamoyl, or ethylmethylcarbamoyl);

Mono- or di-C₆₋₁₄ aryl-carbamoyl (e.g., phenylcarbamoyl, 1-naphthylcarbamoyl, or 2-naphthylcarbamoyl); and
Five- to seven-membered mono- or di-heterocyclic ring-carbamoyl containing one to four of one or two kinds heteroatoms selected from a nitrogen atom, a sulfur atom, and an oxygen atom in addition to carbon atoms (e.g., 2-pyridyl-carbamoyl, 3-pyridyl-carbamoyl, 4-pyridyl-carbamoyl, 2-thienylcarbamoyl, or 3-thienylcarbamoyl).

In addition, for example, any of substituents for the aforementioned “C₆₋₁₄ aryloxy-carbonyl which may be substituted”,
“C₇₋₁₆ aralkyloxy-carbonyl which may be substituted”,
“C₃₋₈ cycloalkyl which may be substituted”,
“C₆₋₁₄ aryl which may be substituted”,
“C₇₋₁₆ aralkyl which may be substituted”,
“C₆₋₁₄ aryl-C₂₋₆ alkenyl which may be substituted”,
“heterocyclic group which may be substituted”,
“C₃₋₈ cycloalkoxy which may be substituted”,
“C₆₋₁₄ aryloxy which may be substituted”,
“C₇₋₁₆ aralkyloxy which may be substituted”,
“C₆₋₁₄ aryl-carbonyloxy which may be substituted”,
“mono- or di-C₆₋₁₄ arylcarbamoyloxy which may be substituted”,
“heterocyclic oxy which may be substituted”,
“aromatic heterocyclic oxy which may be substituted”,
“C₃₋₈ cycloalkylthio which may be substituted”,
“C₆₋₁₄ arylthio which may be substituted”,
“C₇₋₁₆ aralkylthio which may be substituted”,
“heterocyclic thio which may be substituted”,
“C₃₋₈ cycloalkyl-carbonyl which may be substituted”,
“C₆₋₁₄ aryl-carbonyl which may be substituted”,
“C₇₋₁₆ aralkyl-carbonyl which may be substituted”,
“heterocyclic carbonyl which may be substituted”,
“C₃₋₈ cycloalkylsulfonyl which may be substituted”,
“C₆₋₁₄ arylsulfonyl which may be substituted”,
“heterocyclic sulfonyl which may be substituted”,
“C₃₋₈ cycloalkylsulfinyl which may be substituted”,
“C₆₋₁₄ arylsulfinyl which may be substituted”,
“heterocyclic sulfinyl which may be substituted”,
“Carbamoyl group which may be substituted”, and
“Amino group which may be substituted” may be selected from Substituent Group B as listed above and Substituent Group B′ as listed below. In each case, the number of the substituents may be 1 to a maximum substitutable number, preferably 1 to 3, more preferably 1.

### <Substituent Group B′>

C₁₋₆ alkyl, which may be substituted with a halogen atom, hydroxy, cyano, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl; C₇₋₁₆ aralkyl-carbonyl, C₁₋₆ alkoxycarbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkylcarbamoyl, mono- or di-C₆₋₁₄ arylcarbamoyl, or the like);

C₂₋₆ alkenyl, which may be substituted with a halogen atom, hydroxy, cyano, amino, mono- or di-C₁₋₆ alkylamino, mono- or di-C₆₋₁₄ arylamino, mono- or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxycarbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkylcarbamoyl, mono- or di-C₆₋₁₄ arylcarbamoyl, or the like);

C₂₋₆ alkynyl, which may be substituted with a halogen atom, hydroxy, cyano, amino, mono- or di-C₁₋₆ alkylamino, mono- or di-C₆₋₁₄ arylamino, mono-.or di-C₇₋₁₆ aralkylamino, C₃₋₈ cycloalkyl, C₁₋₆ alkoxy, formyl, C₁₋₆ alkyl-carbonyl, C₃₋₈ cycloalkyl-carbonyl, C₆₋₁₄ aryl-carbonyl, C₇₋₁₆ aralkyl-carbonyl, C₁₋₆ alkoxy-carbonyl, C₆₋₁₄ aryloxy-carbonyl, C₇₋₁₆ aralkyloxy-carbonyl, C₁₋₆ alkylthio, C₁₋₆ alkylsulfinyl, C₁₋₆ alkylsulfonyl, carbamoyl, thiocarbamoyl, mono- or di-C₁₋₆ alkyl-carbamoyl, mono- or di-C₆₋₁₄ aryl-carbamoyl, or the like).

R^{a} is preferably a hydrogen atom or a C₁₋₆ alkyl group (preferably methyl).

In the above formula, B represents hydrogen or a substituent.

Examples of the substituent represented by B include any substituent selected from Substituent Group A as described above.

Alternatively, when A is —CONR^{a}—, R^{a} and B may form together with an adjacent nitrogen atom a nitrogen-containing heterocyclic group which may have one or more substituents;

The substituent of the “nitrogen-containing heterocyclic group which may have one or more substituents” may be any substituent selected from Substituent Group A as described above.

Further alternatively, when A is —CONR^{a}—, B may bond to the carbon atom adjacent to the carbon atom to which -A-B is attached to form a five- or six-membered ring which may have one or more substituents. In this case, ring Cy1 is preferably a benzene ring. Examples of the “five- or six-membered ring” include the same five- or six-membered ring which contains at least one nitrogen atoms among the below “five- or six-membered ring” for Cy3.

B is preferably
(1) a hydrogen atom;
(2) a C₁₋₆ alkyl group ((preferably, a C₁₋₄ alkyl group such as methyl, ethyl, propyl, isopropyl, butyl, isobutyl, sec-butyl, or tert-butyl) which may have one or more substituents selected from

(a) a cyano group,

(b) a hydroxy group,

(c) a C₁₋₆ alkoxy group (preferably, methoxy),

(d) a C₆₋₁₄ aryloxy group (preferably, phenoxy),

(e) a carbamoyl group,

(f) an amino group which may have one or two substituents selected from a C₁₋₆ alkyl group (preferably, methyl or isopropyl), a C₆₋₁₄ aryl group (preferably, phenyl), and a C₁₋₆ alkyl-carbonyl group (preferably, acetyl)),

(g) a C₆₋₁₄ aryl group (preferably, phenyl) which may be substituted with an amino group which may be substituted with one or two C₁₋₆ alkyl groups (preferably, methyl);

(h) a five- or six-membered heterocyclic group which may be substituted with at least one (preferably one) substituent selected from a C₁₋₆ alkyl group (preferably, methyl) and an oxo group (examples of such a five- or six-membered heterocyclic group preferably include pyridinyl, tetrahydrofuryl, thienyl, imidazolyl, triazolyl, pyrazolyl, pyridyl, pyrazinyl, morpholinyl, and tetrahydropyranyl),

(i) a C₁₋₆ alkylsulfanyl group (preferably, methylsulfanyl),

(j) a C₁₋₆ alkylsulfinyl group (preferably, methylsulfinyl), and

(k) a C₁₋₆ alkylsulfonyl group (preferably, methylsulfonyl);
(3) a C₃₋₆ cycloalkyl group (preferably, cyclopropyl, cyclopentyl, or cyclohexyl) which may be substituted with at least one (preferably one) hydroxy group;
(4) a C₆₋₁₄ aryl group (preferably, phenyl) which may be substituted with at least one (preferably one) five- or six-membered heterocyclic group (preferably, morpholinyl); or
(5) a 5- to 10-membered heterocyclic group (preferably, thiazolyl, tetrahydropyranyl, pyridyl, morpholinyl, or quinolinyl) which may be substituted with at least one (preferably one) halogen atom (preferably fluorine).

Alternatively, when A is —CONR^{a}—, it is also preferable that R^{a} and B may form together with an adjacent nitrogen atom a six-membered nitrogen-containing heterocyclic group which may have at least one (preferably one) substituent selected from a hydroxy group, a C₁₋₆ alkyl group (preferably, methyl), and a carbamoyl group.

In the above formula, the ring Cy1 represents a six-membered aromatic ring which may have an additional substituent in addition to a group represented by -A-B.

Examples of the “six-membered aromatic ring” represented by the ring Cy1 include (1) a benzene ring and (2) a nitrogen-containing six-membered aromatic heterocyclic ring having at least one (preferably one or two) nitrogen atoms as a ring-constituting element in addition to carbon atoms (e.g., pyridine, pyridazine, pyrimidine, or pyrazine).

The “six-membered aromatic ring” represented by the ring Cy1 is preferably a benzene ring or a pyrimidine ring.

The substituent of the “six-membered aromatic ring which may have an additional substituent in addition to a group represented by -A-B”, which is represented by the ring Cy1, may be a substituent selected from, for example, Substituent Group A as described above. Preferably, examples of the substituent include C₁₋₆ alkyl (e.g., methyl), and halogen (e.g., chlorine or fluorine).

The “six-membered aromatic ring” represented by the ring Cy1 may have one or more such substituents (preferably one or two substituents, more preferably one substituent) on a substitutable position).

The ring Cy1 is preferably unsubstituted. [0097]

The ring Cy1 is preferably a nitrogen-containing six-membered heterocyclic ring that contains benzene or one or two nitrogen atoms.

The ring Cy1 is more preferably benzene or pyridine.

In the above formula, the ring Cy2 represents a six-membered ring which may have one or more substituents selected from
a halogen atom,
a cyano group,
a hydroxy group,
a hydrocarbon-oxy group which may have one or more substituents,
chain hydrocarbon group which may have one or more substituents (preferably, except for a methyl group substituted with a five-membered heterocyclic group),
a heterocyclic group which may have one or more substituents,
an amino group which may have one or more substituents,
an acyl group, and
a carboxy group which may be esterified.

Examples of the “six-membered ring” represented by the ring Cy2 include (1) a carbon ring having six carbons (e.g., cyclohexane, cyclohexene, cyclohexadiene, or benzene) and (2) a six-membered heterocyclic ring having one to three hetero carbons selected from a nitrogen atom, a sulfur atom, and an oxygen atom in addition to carbon atoms (e.g., six-membered aromatic heterocyclic ring such as dihydrofuran, tetrahydrofuran, dihydrothiophene, tetrahydrothiophene, pyrrolidine, pyrroline, pyrazolidine, piperidine, piperazine, morpholine, or thiomorpholine; and a nitrogen-containing six-membered aromatic heterocyclic ring containing pyridine, pyridazine, pyrimidine, or pyrazine. The “six-membered ring” represented by the ring Cy2 is more preferably benzene or pyridine.

Examples of the “hydrocarbon-oxy group” of the “hydrocarbon-oxy group which may have one or more substituents” to be provided as a substituent which the “six-membered ring” represented by the ring Cy2 may have include lower alkoxy, C₃₋₈ cycloalkoxy, C₆₋₁₄ aryloxy, and C₇₋₁₆ aralkyloxy.

The “lower alkoxy” may have one or more substituents (preferably one to three substituents) selected from Substituent Group B as described above.

Each of the “C₃₋₈ cycloalkoxy”, “C₆₋₁₄ aryloxy”, and “C₇₋₁₆ aralkyloxy” may have one or more substituents (preferably one to three substituents) selected from Substituent Group B and Substituent Group B′ as described above.

Examples of the “chain hydrocarbon group” of the “chain hydrocarbon group which may have one or more substituents” to be provided as a substituent which the “six-membered ring” represented by the ring Cy2 may have include lower alkyl, lower alkenyl, and lower alkynyl.

Each of the “lower alkyl”, the “lower alkenyl”, and the “lower alkynyl” may have one or more (preferably one to three) substituents selected from Substituent Group B as described above.

Examples of the “amino group which may have one or more substituents” to be provided as a substituent which the “six-membered ring” represented by the ring Cy2 may have include the same substituents as those of the “amino group which may have one or more substituents” in Substituent Group A as described above.

Examples of the “heterocyclic group which may have one or more substituents” to be provided as a substituent which the “six-membered ring” represented by the ring Cy2 may have include the same substituents as those of the “heterocyclic group which may be substituted” in Substituent Group A as described above.

Examples of the “acyl group” to be provided as a substituent which the “six-membered ring” represented by the ring Cy2 include formyl, lower alkyl-carbonyl, C₁₋₆ alkyl-carbonyl, C₃₋₈ cycloalkyl-carbonyl, C₆₋₁₄ aryl-carbonyl, and C₇₋₁₆ aralkyl-carbonyl.

The “six-membered ring” represented by the ring Cy2 may have at least one (preferably one) substituent selected from a halogen atom (e.g., fluorine); an alkyl group which may have one or more substituents (preferably, the alkyl group is a C₁₋₃ alkyl group (e.g., methyl, ethyl, propyl, or isopropyl) which may be halogenated); and an alkoxy group which may have one or more substituents (preferably, the alkoxy group is a C₁₋₃ alkoxy group (e.g., methoxy, ethoxy, propoxy, isopropoxy), more preferably methoxy) which may be substituted one or more substituents. The “six-membered ring” represented by the ring Cy2, is also preferably unsubstituted.

In the above formula, ring Cy3 may represent a five- or six-membered ring which may have one or more substituents.

Examples of the “five- or six-membered ring” represented by the ring Cy3 include: (1) a carbon ring having 5 to 6 carbon atoms (e.g., cyclopentane, cyclopentene, cyclopentadiene, cyclohexane, cyclohexene, cyclohexadiene, or benzene); and (2) a five- or six-membered monoheterocyclic aromatic ring (e.g., furan, thiophene, pyrrole, oxazole, isoxazole, thiazole, isothiazole, imidazole, pyrazole, 1,2,3-oxadiazole, 1,2,4-oxadiazole, 1,3,4-oxadiazole, furazan, 1,2,3-thiadiazole, 1,2,4-thiadiazole, 1,3,4-thiadiazole, 1,2,3-triazole, 1,2,4-triazole, tetrazole, pyridine, pyridazine, pyrimidine, pyrazine, or triazine). The “five- or six-membered ring” represented by the ring Cy3 is preferably dihydrofuran, furan, oxazole, dihydropyrrole, pyrazole, imidazole, triazole, thiazole, or tetrahydropyridine.

The substituent of the “five- or six-membered ring which may have one or more substituents” represented by the ring Cy3 may be, for example, any substituent selected from Substituent Group A as described above. The number of substituents which the “five- or six-membered ring” represented by the ring Cy3 may have is preferably zero (i.e., unsubstituted) or one.

Preferably, examples of the substituent of the “five- or six-membered ring which may have one or more substituents” represented by the ring Cy3 include a halogen atom, an alkyl group which may have one or more substituents (preferably, a C₁₋₃ alkyl group which may be halogenated (e.g., methyl, ethyl, propyl, or isopropyl, more preferably methyl) and an alkoxy group which may have one or more substituents (preferably, a C₁₋₃ alkoxy group (e.g., methoxy, ethoxy, propoxy, or isopropoxy)) which may be halogenated.

In the above formula, a moiety represented by the chemical formula:

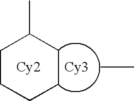

is preferably, for example, any of the following formulae:

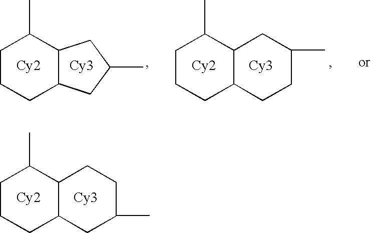

In other words, the formula (I) is preferably as follows:

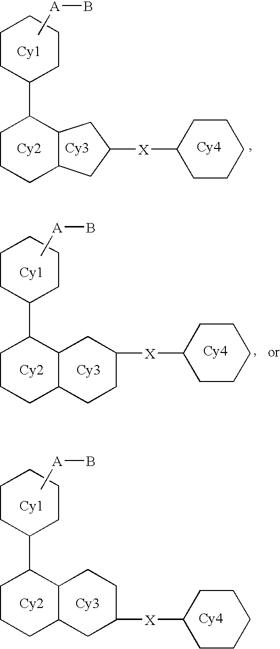

Here, the chemical formula (I) is preferably as follows:

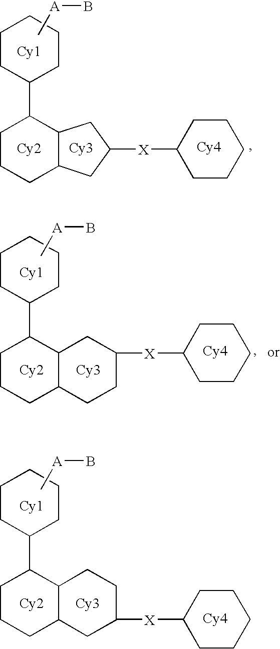

wherein

ring Cy1 is a benzene ring or a pyrimidine ring;

ring Cy2 is a six-membered ring which may have one or more substituents selected from a halogen atom, an alkyl group which may have one or more substituents, and an alkoxy group which may have one or more substituents;

ring Cy3 is a five- or six-membered ring which may have one or more substituents selected from a halogen atom, an alkyl group which may have one or more substituents, and an alkoxy group which may have one or more substituents; and

ring Cy 4 is a benzene ring or a pyrimidine ring, which may have one or more substituents. Examples of the skeleton (i.e., moiety other than a substituent) of the moiety represented as follows:

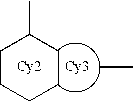

include

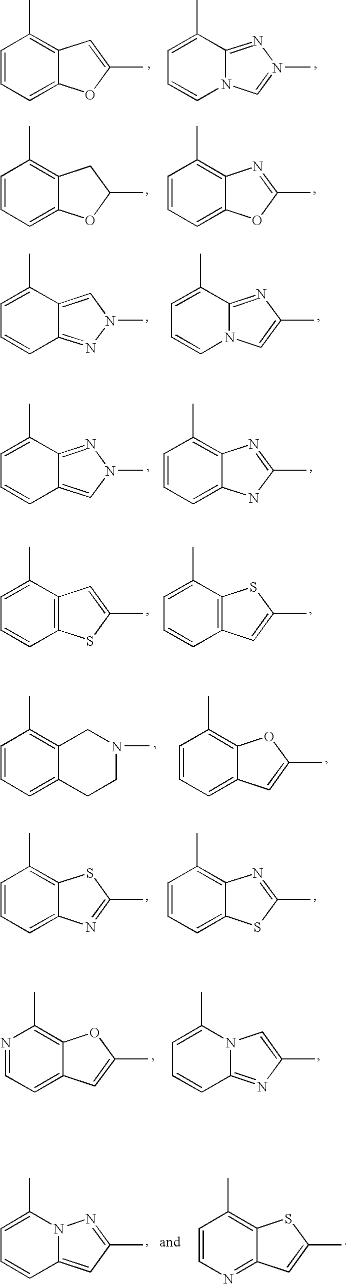

The moiety is preferably, for example,

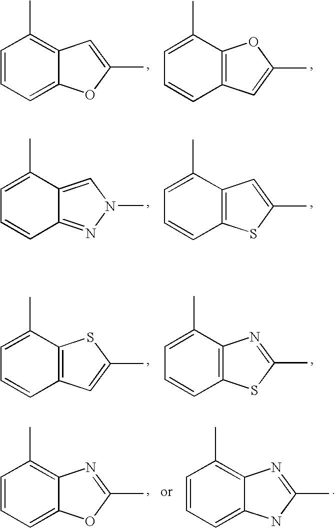

The moiety is more preferably, for example,

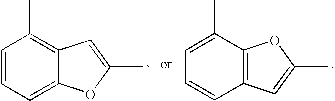

The moiety is also preferably, for example,

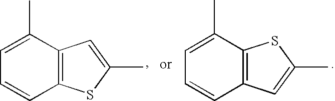

In the above formula, X represents C₁₋₂ alkylene (e.g., methylene, ethylene, methylmethylene) which may be substituted with hydroxy, —Y—, —Y—CH₂—, or —CH₂—Y—.

Here, Y represents —O—, —NR^{b}—, or —S(O)ₘ— and m represents an integer of 0 to 2; R^{b} represents a hydrogen atom or a substituent. The substituent represented by R^{b} may be the same as one represented by R^{a}.

X is preferably, for example, C₁₋₂ alkylene which may be substituted with hydroxy(e.g., —CH₂—, —CH₂—CH₂—, —CH(CH₃)—, —CH(OH)—, —C(CH₃)(OH)—), —NH—, —CH₂—O—, —CH₂—NH—, —CH₂—N(CH₃)—, —O—CH₂—, —S—, or —O—.

X is more preferably, for example, C₁₋₂ alkylene, —CH₂—O—, or —O—, further preferably, C₁₋₂ alkylene, or —O—.

In the above formulae, ring Cy4 represents a six-membered aromatic ring which may have one or more substituents.

Examples of the “six-membered aromatic ring” represented by ring Cy4 include (1) a benzene ring and (2) a six-membered nitrogen-containing aromatic heterocyclic ring (e.g., pyridine, pyridazine, pyrimidine, or pyrazine) that contains at least one (preferably one or two) nitrogen atoms as one of ring-constituting atoms in addition to carbon atoms.

The “six-membered aromatic ring” represented by ring Cy4 is preferably benzene or pyridine, more preferably benzene.

The substituent of the “six-membered aromatic ring which may have one or more substituents” may be, for example, one selected from Substituent Group A as described above (preferably not a sulfamoyl group). The “six-membered aromatic ring” represented by ring Cy4 may have one or more such substituents (preferably one or two, more preferably one) on the substitutable position thereof.

The “six-membered aromatic ring which may have one or more substituents” includes a pyridone ring which may have one or more substituents.

Examples of the substituent include, more preferably, a halogen atom (preferably, chlorine or fluorine), a C₁₋₆ alkyl group which may be halogenated or hydroxylated (preferably, methyl, trifluoromethyl, —CH(OH)CH₃, —CH₂OH,), a C₁₋₆ alkoxy group (preferably, methoxy), a C₁₋₆ alkylsulfonyl group (preferably, methylsulfonyl).

More preferably, these preferable examples may be used in combination.

The compound (I₀) is preferably as follows:

### <Compound Aa>

A compound is one represented by the formula (Ia)

wherein
the chemical formula (Ia) is as follows:

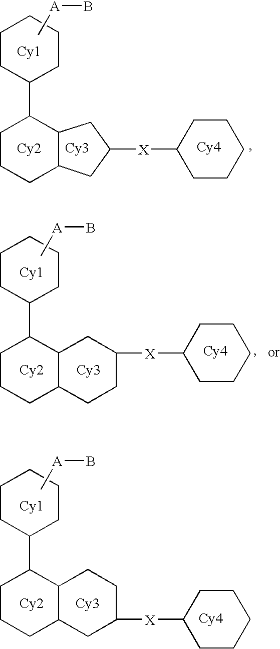

wherein
A represents —CONR^{a}— or —NR^{a}CO—;
R^{a} represents a hydrogen atom or a C₁₋₆ alkyl group a substituent;
B represents
   1) a hydrogen atom,
   2) a C₁₋₆ alkyl group which may have on& or more substituents selected from
      a) cyano group,
      b) a hydroxy group,
      c) a C₁₋₆ alkoxy group,
      d) a C₆₋₁₄ aryloxy group,
      e) a carbamoyl group, and
      f) an amino group which may be substituted with one or two substitutes selected from a C₁₋₆ alkyl group, a C₆₋₁₄ aryl group, a C₁₋₆ alkylcarbonyl group,
      g) a C₆₋₁₄ aryl group which may be substituted with an amino-group which may be substituted with one or two C₁₋₆ alkyl group,
      h) a five- or six-membered heterocyclic group which may be substituted with a substituent selected from a C₁₋₆ alkyl group and an oxo group,
      i) a C₁₋₆ alkylsulfanyl group,
      j) a C₁₋₆ alkylsulfinyl group, and
      k) a C₁₋₆ alkylsulfonyl group,
   3) a C₃₋₆ cycloalkyl group which may be substituted with a hydroxy group,
   4) a C₆₋₁₄ aryl group which may be substituted with a five- or six-membered heterocyclic group, or
   5) a five- to ten-membered heterocyclic group which may be substituted with a halogen atom,
or alternatively, when A is —CONR^{a}—, R^{a} and B may form together with an adjacent nitrogen atom a nitrogen-containing six-membered heterocyclic group which may have one or more substituents selected from a hydroxy group, a C₁₋₆ alkyl group, and a carbamoyl group;
ring Cy1 represents a benzene ring or a pyridine ring, each of which may have one or more substituents selected from a halogen atom and a C₁₋₆ alkyl group;
ring Cy2 represents a benzene ring or a pyridine ring which may have one or more substituents selected from a halogen atom and a C₁₋₆ alkoxy group;
ring Cy3 represents a five- or six-membered heterocyclic ring which may have one or more substituents selected from
   1) a C₁₋₆ alkyl group
   2) an oxo group and
   3) a halogen atom;
X represents —CH₂—, —CH₂—CH₂—, —CH(CH₃)—, —NH—, —CH(OH)—, —CH₂—O—, —C(CH₃)(OH)—, or —O—; and
ring Cy4 represents
   1) a benzene ring which may have one or more substituents selected from
      a) a halogen atom,
      b) a C₁₋₆ alkyl group which may be halogenated or hydroxylated,
      c) a C₁₋₆ alkoxy group
      d) an amino group which may be substituted with one or two C₁₋₆ alkyl groups, and
      e) a C₁₋₆ alkylsulfonyl group,
   2) a pyridine ring which may have one or more substituents selected from
      a) a C₁₋₆ alkyl group which may be halogenated, and
      b) a C₁₋₆ alkoxy,
   3) -a pyridone ring which may have one or more substituents selected from
      a) a halogen atom, and
      b) a C₁₋₆ alkyl group which may be halogenated.
Here, more preferably, the chemical formula (Ia) is the chemical formula (II)

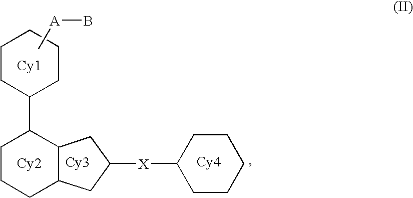

A is —CONR^{a},
R^{a} is a hydrogen atom, or a C₁₋₆ alkyl group,
B is
1) a hydrogen atom,
2) a C₁₋₆ alkyl group,
   which may have one or more substituents selected from
a) a cyano group,
b) a hydroxy group,
c) a C₁₋₆ alkoxy group,
d) a carbamoyl group,
e) an amino group which may have one or two substituents selected from a C₁₋₆ alkyl group, a C₆₋₁₄ aryl group, and a C₁₋₆ alkyl-carbonyl group
f) a C₁₋₆ alkylsulfinyl group,
ring Cy1 is a benzene ring or a pyridine ring
ring Cy2 is a benzene ring or a pyridine ring
   which may have one or more substituents selected from
a halogen atom and a C₁₋₆ alkoxy group,
ring Cy3 is a 5-membered heterocyclic ring
which may have one or more substituents selected from
C₁₋₆ alkyl group, and an oxo group
X is C₁₋₂ alkylene, or —O—
ring Cy4 is
a benzene ring
   which may have one or more substituents selected from
1) a halogen atom,
2) a C₁₋₆ alkyl group which may be halogenated,
3) a C₁₋₆ alkoxy group, and
4) a C₁₋₆ alkylsulfonyl group
   The compound (I₀) is preferably as follows:

### <Compound A>

A compound is one represented by the following formula (I:

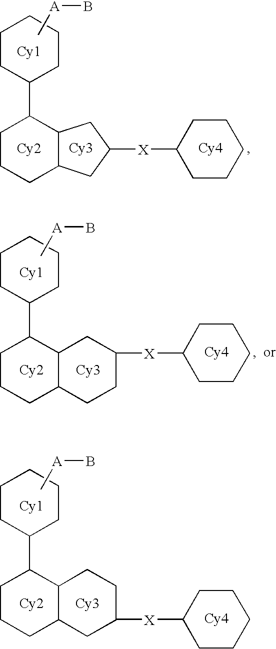

wherein
A represents —CONR^{a} or —NR^{a}CO—;
R^{a} represents a hydrogen atom or a C₁₋₆ alkyl group a substituent;
B represents
(1) a hydrogen atom,
(2) a C₁₋₆ alkyl group which may have one or more substituents selected from
   a) a cyano group,
   b) a hydroxy group,
   c) a C₁₋₆ alkoxy group,
   d) a C₆₋₁₄ aryloxy group,
   e) a carbamoyl group,
   f) an amino group which may have one or more substituents selected from a C₁₋₆ alkyl group, a C₆₋₁₄ aryl group, and a C₁₋₆ alkyl-carbonyl group,
   g) a C₆₋₁₄ aryl group which may be substituted with an amino group which may be substituted with one or two C₁₋₆ alkyl groups,
   h) a five- or six-membered heterocyclic group which may be substituted with a substituent selected from a C₁₋₆ alkyl group, and an oxo group,
   i) a C₁₋₆ alkyl sulfanyl group,
   j) a C₁₋₆ alkyl sulfinyl group, and
   k) a C₁₋₆ alkyl sulfonyl group,
(3) a C₃₋₆ cycloalkyl group which may be substituted with a hydroxy group,
(4) a C₆₋₁₄ aryl group, or
(5) a five- to ten-membered heterocyclic group which may be substituted with a halogen atom,
   or alternatively, when A is —CONR^{a}—, R^{a} and B may form, together with an adjacent nitrogen atom, a six-membered nitrogen heterocyclic group which may have one or more substituents selected from a hydroxy group, a C₁₋₆ alkyl group, and a carbamoyl group,

a ring Cy1 represents a benzene ring or a pyrimidine ring,

a ring Cy2 represents a benzene ring or a pyrimidine ring which may have one or more substituents selected from a halogen atom and a C₁₋₆ alkoxy group,

a ring Cy3 represents a five- or six-membered heterocyclic ring which may have one or more substituents selected from a C₁₋₆ alkyl group and an oxo group,

X represents a C₁₋₂ alkylene or —NH—,

a ring Cy4 represents a benzene ring which may have one or more substituents selected from a halogen atom, a C₁₋₆ alkyl group which may be halogenated, and a C₁₋₆ alkoxy group; or a salt thereof.

Here, more preferably, the chemical formula (I) is the chemical formula(II)

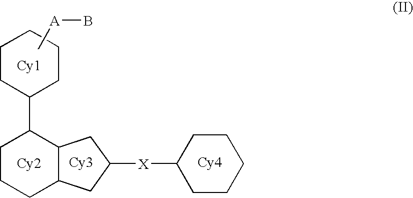

A is —CONR^{a},
R^{a} is a hydrogen atom, or a C₁₋₆ alkyl group,
B is
1) a hydrogen atom,
2) a C₁₋₆ alkyl group,
   which may have one or more substituents selected from
a) a cyano group,
b) a hydroxy group,
c) a C₁₋₆ alkoxy group,
d) a carbamoyl group,
e) an amino group, which may have one or two substituents selected from a C₁₋₆ alkyl group, a C₆₋₁₄ aryl group, and a C₁₋₆ alkyl-carbonyl group
f) a C₁₋₆ alkylsulfinyl group,
ring Cy1 is a benzene ring, or a pyridine ring,
ring Cy2 is a benzene ring, or a pyridine ring which may have one or more substituents selected from
a halogen atom, and a C₁₋₆ alkoxy group,
ring Cy3 is a 5-membered heterocyclic ring which may have one or more substituents selected from
C₁₋₆ alkyl group, and an oxo group,
X is
C₁₋₂ alkylene, or —O—,
ring Cy4 is
a benzene ring
   which may have one or more substituents selected from
1) a halogen atom,
2) C₁₋₆ alkyl group which may be halogenated, and
3) a C₁₋₆ alkoxy group,

Here, further preferably, the chemical formula (I) is the chemical formula (III)

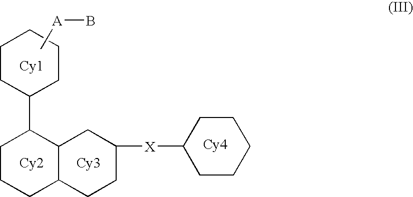

A is —CONR^{a},
R^{a} is a hydrogen atom,
B is
C₁₋₆ alkyl group which may have one or more substituents selected from
   a) a cyano group, and
   b) a hydroxy group,
ring Cy1 is a benzene ring
the skeleton of the moiety represented by

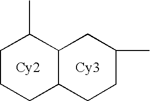

of the chemical formula (III) is a fused ring represented by

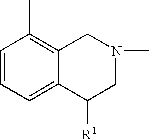

wherein R¹ is a hydrogen atom, or a C₁₋₆ alkyl group
X is C₁₋₂ alkylene,
ring Cy4 is a benzene ring which is substituted with C₁₋₆ alkyl group which may be halogenated

Here, preferably, the compound (I₀) is not a compound represented by the following formula:

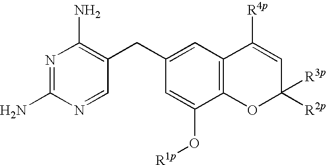

wherein
R^{1p} represents alkyl or cycloalkylalkyl;
R^{2p} and R^{3p} each independently represent an alkyl or a cycloalkyl or represent, together with an adjacent carbon atom, any of saturated three- to six-membered carbon rings or heterocyclic rings (where alkyl, cycloalkyl, a carbon ring, or a heterocyclic ring is unsaturated or saturated), and
R^{4p} represents aryl which may be substituted or heteroaryl which may be substituted,
a compound represented by the following formula:

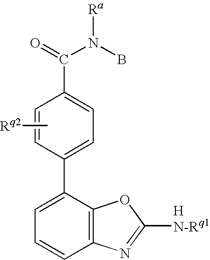

wherein
R^{q1} represents phenyl which may have one or more substituents,
R^{q2} represents hydrogen, or a substituent,
the other symbols are synonymous with those described above,

a compound represented by the following formula:

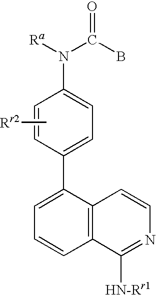

wherein
R^{r1} represents phenyl which may have one or more substituents,
R^{q2} represents hydrogen, or a substituent,
the other symbols are synonymous with those described above,
7-[4-(acetylamino)phenyl]-2-(benzylsulfanyl)-5-methyl-N-phenyl-3,7-dihydro[1,2,4]triazolo[1,5-a]pyrimidine-6- carboxamide,
7-[4-(acetylamino)phenyl]-2-[(4-chlorobenzyl)sulfanyl]-N-(2,4-dimethylphenyl)-5-methyl-3,7-dihydro[1,2,4]triazolo [1,5-a]pyrimidine-6-carboxamide,

7-[4-(acetylamino)phenyl]-2-(benzylsulfanyl)-N-(2-methoxyphenyl)-5-methyl-3,7-dihydro[1,2,4]triazolo[1,5-a]pyrimidine-6-carboxamide,

7- [4-(acetylamino)phenyl]-2-[(2,4-dimethylbenzyl) sulfanyl]-N-(4-methoxyphenyl)-5-methyl-3,7-dihydro[1,2,4]triazolo[1,5-a]pyrimidine-6-carboxamide,

7-[4-(acetylamino)phenyl]-2-(benzylsulfanyl)-N-(2,4-dimethylphenyl)-5-methyl-3,7-dihydro[1,2,4]triazolo [1,5-a]pyrimidine-6-carboxamide,

N-(3-(2-((4-chloro-2-methoxy-6-methylphenyl)amino)-1-methyl-1H-benzoimidazol-7-yl)phenyl)acetamide),

tert-butyl methyl [4-[2-methyl-1-(phenylsulfonyl)-1H-pyrrolo[2,3-b]pyridin-4-yl]phenyl]carbamate,

tert-butyl (4-[3-[(4-methoxybenzyl)amino]imidazo[1,5-a]pyridin-5-yl]phenyl)carbamate,

tert-butyl 1-[4-(diethylcarbamoyl)phenyl]-6-methoxy-7-phenoxy-3,4-dihydroisoquinoline-2(1H)-carboxylate,

tert-butyl 1-[4-(diethylcarbamoyl)phenyl]-7-(4-fluorophenoxy)-6-methoxy-3,4-dihydroisoquinoline-2(1H)-carboxylate,

tert-butyl 1-[4-(diethylcarbamoyl)phenyl]-6-methoxy-7-(4-methoxyphenoxy)-3,4-dihydroisoquinoline-2(1H)-carboxylate,

tert-butyl 1-[4-(diethylcarbamoyl)phenyl]-6-methoxy-7-(pyridin-3-yloxy)-3,4-dihydroisoquinoline-2(1H)-carboxylate,
1-[4-(1-benzyl-1H-pyrazolo[3,4-c]pyridin-4-yl)phenyl]-3-[3-(trifluoromethyl)phenyl]urea,
1-[4-[1-(4-methoxybenzyl)-1H-pyrazolo[3,4-c]pyridin-4-yl]phenyl]-3-[3-(trifluoromethyl) phenyl]urea,
3-chloro-2-[6-[(2-chloro-4-fluorophenyl)sulfanyl]-2-oxo-3,4-dihydropyrido[3,2-d]pyrimidin-1(2H)-yl]benzamide,
3,5-dichloro-4-[6-[(2,4-difluorophenyl)sulfanyl]-2-oxo-3,4-dihydropyrido[3,2-d]pyrimidin-1(2H)-yl]benzamide,
3,5-dichloro-4-[6-[(2,4-difluorophenyl)sulfanyl]-2-oxo-3,4-dihydropyrido[3,2-d]pyrimidin-1(2H)-yl]-N-[2-(dimethylamino)ethyl]benzamide,
2-chloro-N-(3-chloro-4-[2-[(4-fluorophenyl)sulfanyl-6-oxo-7,8-dihydro-6H-pyrimido-8 1,6-b]pyridazin-5-yl]phenyl)acetamide,
N-(3-chloro-4-[2-[(4-fluorophenyl)sulfanyl]-6-oxo-7,8-dihydro-6H-pyrimidol[6-b]pyridazin-5-yl]phenyl)acetamide,
N-(3-chloro-4-[2-[(4-fluorophenyl)sulfanyl]-6-oxo-7,8-dihydro-6H-pyrimido[1,6-b]pyridazin-5-yl]phenyl)-2-morpholin-4-ylacetamide,

N-(4-[2-[(3,4,5-trimethoxyphenyl)amino]-1,3-benzoxazol-7-yl]phenyl)acetamide,

N-(3-[2-[(3,4,5-trimethoxyphenyl)amino]-1,3-benzoxazol-7-yl]phenyl)acetamide,
N-(2-amino-4-[2-[(3,4,5-trimethoxyphenyl)amino]-1,3-benzoxazol-7-yl]phenyl)formamide,
7-[4-(acetylamino)phenyl]-2-[(2,4-dimethylbenzyl)sulfanyl]-5-methyl-N-phenyl-3,7-dihydro[1,2,4]triazolo[1,5-a]pyrimidine-6-carboxamide,
5-[(3S )-3-(dibenzylamino)-3,4-dihydro-2H-chromen-5-yl]-2-methoxy-N,N-dimethylpyridine-3-carboxamide,
5-[(3S)-3-(dibenzylamino)-3,4-dihydro-2H-chromen-5-yl]-2-methoxy-N-methylpyridine-3-carboxamide,
5-[(3S)-3-(dibenzylamino)-3,4-dihydro-2H-chromen-5-yl]-2-methoxy-N-methylpyridine-3-carboxamide, and
N-(6-[1-[(4-methylphenyl)sulfonyl]-1H-pyrrolo[2,3-b]pyridin-4-yl]pyridin-2-yl)acetamide.

Most preferable examples of the compound (I₀) include the following compounds.
N-(2-hydroxyethyl)-3-[2-[3-(trifluoromethyl)benzyl]-1-benzofuran-4-yl]benzamide, or a salt thereof.
N-(2-hydroxyethyl)-3-[2-[3-(trifluoromethyl)benzyl]-1-benzofuran-4-yl]benzamide.
3-[2-(3-chloro-4-fluorobenzyl)-2H-indazol-4-yl]-N-(2-cyanoethyl)benzamide, or a salt thereof.
N-(2-cyanoethyl)-3-(1-methyl-2-[3-(trifluoromethyl)benzyl]-1H-benzimidazol-4-yl)benzamide, or a salt thereof.
N-(2-methoxyethyl)-3-[1-methyl-2-[3-(trifluoromethyl)phenoxy]-1H-benzimidazol-4-yl]benzamide, or a salt thereof.
3-[2-[[3-(trifluoromethyl)phenoxy]methyl]-1-benzothiophen-7-yl]benzamide, or a salt thereof.
3-[2-[[3-(trifluoromethyl)phenoxy]methyl]-1-benzothiophen-7-yl]benzamide.
N-(2-hydroxyethyl)-3-[3-methyl-2-[3-(trifluoromethyl)benzyl]-1-benzothiophen-7-yl]benzamide, or a salt thereof.
N-(2-hydroxyethyl)-3-[3-methyl-2-[3-(trifluoromethyl)benzyl]-1-benzothiophen-7-yl]benzamide.
N-(2-hydroxyethyl)-2-{2-[3-(trifluoromethyl)benzyl]-1-benzothiophen-7-yl}pyridine-4-carboxamide, or a salt thereof.
N-(2-amino-2-oxoethyl)-3-[4-fluoro-2-[3-(trifluoromethyl)benzyl]-1-benzothiophen-7-yl]benzamide, or a salt thereof.
N-(2-amino-2-oxoethyl)-3-[4-fluoro-2-[3-(trifluoromethyl)benzyl]-1-benzothiophen-7-yl]benzamide.
N-(2-amino-2-oxoethyl)-3-[2-[3-(trifluoromethyl)benzyl]-1,3-benzothiazol-4-yl]benzamide, or a salt thereof.
3-[2-(3-chloro-5-fluorobenzyl)-1-benzothiophen-7-yl]-N-[2-(1-methylethoxy)ethyl]benzamide, or a salt thereof.
3-[2-(3-chloro-5-fluorobenzyl)-1-benzothiophen-7-yl]-N-[2-(1-methylethoxy)ethyl]benzamide.

When the compound (I₀) is a salt, examples of such a salt include metal salt, ammonium salt, salt with organic base, salt with inorganic acid, salt with organic acid, salt with basic or acidic amino salt. Preferable examples of the metal salt include alkaline metal salts such as sodium salt and potassium salt; alkaline earth metal salts such as calcium salt, magnesium base, and barium salt; and aluminum salt. Preferable examples of the salt with organic base include salts with trimethyl amine, triethyl amine, pyridine, picoline, 2,6-lutidine, ethanol amine, diethanol amine, triethanol amine, cyclohexyl amine, dicyclohexyl amine, and N,N′-dibenzyl ethylene diamine. Preferable examples of the sat with inorganic acid include salts with hydrochloric acid, hydrobromic acid, nitric acid, sulfuric acid, and phosphoric acid. Preferable examples of the salt with organic acid include salts with salts with formic acid, acetic acid, trifluoroacetic acid, phthalic acid, fumaric acid, oxalic acid, tartaric acid, maleic acid, citric acid, succinic acid, malic acid, methane sulfonic acid, benzene sulfonic acid, and p-toluene sulfonic acid. Preferable examples of the salt with basic amino acid include salts with arginine, lysine, and ornithine. Preferable examples of the salt of acid amino acid include salt with aspartic acid and glutaminic acid. Among them, pharmaceutically acceptable salts are preferable. For example, if the compound has an acidic functional group therein, examples of the salt include inorganic salt such as alkaline salt (e.g., sodium salt and potassium salt) and alkaline earth metal salt (e.g., calcium salt, magnesium salt, and barium salt); and ammonium salt. If the compound has a basic functional group therein, examples of the salt thereof include inorganic acids such as hydrochloric acid, hydrobromic acid, nitric acid, and phosphoric acid, or salts with acetic acid, phthalic acid, fumaric acid, oxalic acid, tartaric aid, maleic acid, citric aid, succinic acid, methane sulfonic acid, and p-toluene sulfonic acid.

When there is an isomer of the compound (I₀), such as a tautomer, an optical isomer, a stereoisomer, a positional isomer, or a rotational isomer, an isomer may be present or alone or in combination and provided as a compound of the present invention. Furthermore, if there is an optical isomer of the compound (I₀), an optical isomer isolated from a racemic mixture is also provided as the compound (I₀).

The compound (I₀) may be a crystallized compound. Even if the compound (I₀) is in single crystal form or mixed crystal form, it can be provided as the compound (I₀) of the present invention.

The compound (I₀) may be a solvate (e.g., a hydrate) or a nonsolvate. Any of them can be provided as the compound (I₀) of the present invention.

Any of the above compounds may be labeled or substituted with an isotope (e.g., ²H, ³H, ¹¹C, ¹⁴C, ¹⁸F, ³⁵S, or ¹²⁵I) and provided as the compound (I₀) of the present invention.

### <Manufacturing Method>

Hereinafter, a method for manufacturing the compound of the present invention will be described.

For example, the compound (I₀) can be obtained by a process represented by a reaction formula described below or another process based thereof. The symbols for the compounds in the reaction formula are synonymous with those described above. Here, the compounds in the formula may also represent those forming salts. Examples of such salts are same as those of the compound (I₀). In addition, compounds obtained in the respective steps may be directly used as a reaction solution or a crude product in the subsequent reaction. Alternatively, it may be isolated from the reaction mixture by a conventional method and can be easily purified by any of well-known separation techniques, such as extraction, concentration, neutralization, filtration, distillation, recrystallization, and chromatography. Alternatively, if the compound in the formula is commercially available, a corresponding commercial product may be directly used.

The compound (I₀) can be produced by the process represented by Reaction Formula I as follows.

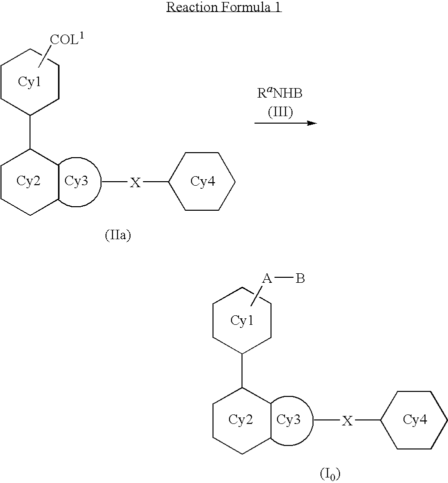

In the reaction formula, L¹ represents a leaving group.

The compound (I₀) can be produced by reaction of a compound (IIa) with a compound (III) in the presence of base or acid if desired.

The compound (III) may be a commercially available product or may be produced according to a well-known method or another method based thereon.

Examples of the “leaving group” represented by L¹ include a hydroxy group, a halogen atom (e.g. fluorine, chlorine, bromine, iodine), C₁₋₆ alkoxy (e.g., methoxy, ethoxy, propoxy, isopropoxy, butoxy, isobutoxy, sec-butoxy, pentyloxy, or hexyloxy) which may be halogenated, a C₁₋₆ alkylsulfonyloxy group (e.g., methane sulfonyloxy, ethane sulfonyloxy, or trichloromethane sulfonyloxy) which may be substituted, a C₆₋₁₀ arylsulfonyloxy group which may be substituted, a phenyloxy group which may be substituted, or a benzothiazol-2-yl thio group which may be substituted.

Examples of the “C₆₋₁₀ arylsulfonyloxy group which may be substituted” include a C₆₋₁₀ arylsulfonyloxy group (e.g., phenylsulfonyloxy, or naphthylsulfonyloxy) which may have one to three substituents selected from a C₁₋₆ alkyl (e.g., methyl, ethyl, propyl, isopropyl, butyl, isobutyl, sec-butyl, tert-butyl, pentyl, or hexyl), C₁₋₆ alkoxy (e.g., methoxy, ethoxy, propoxy, isopropoxy, butoxy, isobutoxy, sec-butoxy, pentyloxy, or hexyloxy), and nitro. Specific examples include benzene sulfonyloxy, m-nitrobenzene sulfonyloxy, and p-toluene sulfonyloxy.

Examples of the “phenyloxy group which may substituted” include a phenyloxy group which may have one to three substituents selected from C₁₋₆ alkyl (e.g., methyl, ethyl, propyl, isopropyl, butyl, isobutyl, sec-butyl, tert-butyl, pentyl, or hexyl), C₁₋₆ alkoxy (e.g., methoxy, ethoxy, propoxy, isopropoxy, butoxy, isobutoxy, sec-butoxy, pentyloxy, or hexyloxy), and nitro. Specific examples include phenyloxy, and 4-nitrophenoxy.

Examples the “benzothiazol-2-yl thio group which may be substituted” include a benzothiazol-2-yl thio group which may have one to three substituents selected from C₁₋₆ alkyl (e.g., methyl, ethyl, propyl, isopropyl, butyl, isobutyl, sec-butyl, tert-butyl, pentyl, or hexyl), C₁₋₆ alkoxy (e.g., methoxy, ethoxy, propoxy, isopropoxy, butoxy, isobutoxy, sec-butoxy, pentyloxy, or hexyloxy), and nitro. Specific examples include benzothiazol-2-yl thio.

The amount of the compound (III) used is about 1 to 10 mol, preferably 1 to 2 mol per mol of the compound (IIa).

Examples of the “base” include basic salts such as sodium carbonate, potassium carbonate, cesium carbonate, and sodium bicarbonate; aromatic amines such as pyridine and lutidine; tertiary amines such as triethyl amine, diisopropylethylamine, tripropyl amine, tributyl amine, cyclohexyldimethyl amine, 4-dimethylaminopyridine, N-methylpiperidine, N-methylpyrrolidine, and N-methylmorpholine; alkaline metal hydrides such as sodium hydride and potassium hydride; metal amides such as sodium amide, lithium diisopropyl amide, and lithium hexamethyldisilazide; and metal alkoxides such as sodium methoxide, sodium ethoxide, and sodium tert-butoxide.

The amount of the “base” used is generally about 0.1 to 10, preferably 0.8 to 2 equivalents per compound (IIa). Examples of the “acid” include methane sulfonic acid, p-toluene sulfonic acid, and camphor sulfonic acid.

The amount of the “acid” used is generally about 0.1 to 10, preferably 0.8 to 3 equivalents per compound (IIa).

It is advantageous to carry out the present reaction in the absence of a solvent or in the presence of a solvent inactive to the reaction. Preferable examples of such a solvent include, but not specifically limited as long the reaction proceeds, water; ethers such as diethyl ether, tetrahydrofuran, 1,4-dioxane, and 1,2-dimethoxy ethane; hydrocarbons such as benzene, toluene, cyclohexane, and hexane; amides such as N,N-dimethyl formamide and N,N-dimethyl acetamide; halogenated hydrocarbons such as dichloromethane, chloroform, carbon tetrachloride, and 1,2-dichloroethane; nitriles such as acetonitrile and propionitrile; sulfoxides such as dimethyl sulfoxide; and nitrogen-containing aromatic hydrocarbons such as pyridine, lutidine, and quinoline, or mixtures thereof.

The reaction temperature is generally in the range of −40 to 150° C., preferably 0 to 100° C.

The reaction time is generally in the range of 5 minutes to 24 hours, preferably 10 minutes to 5 hours.

If L¹ is OH, as an alternative method, the compound (IIa) may be reacted with the compound (III) in the presence of an appropriate condensation agent.

The amount of the compound (III) used is generally about 0.8 to 10 mol, preferably about 0.8 to 2 mol per mol of the compound (IIa).

Examples of the “condensation agent” include: N,N′-carbodiimides such as N,N′-dicyclohexyl carbodiimide and 1-ethyl-3-(3-dimethylaminopropyl)carbodiimide hydrochloride salt (WSC); azorites such as N,N′-carbonylimidazole; 2- halogeno pyridinium salts such as 2-chloro-1-methyl pyridinium iodide and 2-fluoro-1-methyl pyridinium iodide; and other compounds such as N-ethoxycarbonyl-2-ethoxy-1,2-dihydroquinoline, 2-(7-aza-1H-benzotriazol-1-yl)-1,1,3,3-tetramethyluronium hexafluorophosphate (HATU), 4-(4,6-dimethoxy-1,3,5-triazin-2-yl)-4-methylmorpholinium chloride n-hydrate (DMTMM), diethyl cyanophosphate, phosphorous oxychloride, and acetic anhydride.

The amount of the “condensation agent” used is generally about 0.8 to 5 mol, preferably about 1 to 3 mol per compound (IIa).

The reaction may be carried out in the presence of base. Examples of the “base” include basic salts such as potassium acetate and sodium acetate; and tertiary amines such as triethyl amine, diisopropylethylamine, tripropyl amine, tributyl amine, cyclohexyl dimethyl amine, 4-dimethylaminopyridine, N-methylpiperidine, N-methylpyrrolidine, and N-methylmorpholine. In addition, if required, the reaction may be carried out in the presence of a condensation accelerator such as 1-hydroxy-1H-benzotriazole(HOBt) monohydrate.

The amount of “base” used is generally about 0.5 to 5 mol, preferably about 2 to 3 mol per mol of the compound (IIa).

It is advantageous to carry out the present reaction using a solvent inactive to the reaction. Preferably, examples of such a solvent include: alcohols such as methanol, ethanol, and propanol; hydrocarbons such as hexane, cyclohexane, benzene, toluene, and xylene; ethers such as diethyl ether, diisopropyl ether, tetrahydrofuran, 1,4-dioxane, and 1,2-dimethoxy ethane; amides such as N,N-dimethyl formamide, N,N-dimethyl acetamide, hexamethyl phosphoric triamide, and 1-methyl pyrrolidine-2-one; sulfoxides such as dimethyl sulfoxide; halogenated hydrocarbons such as dichloromethane, chloroform, carbon tetrachloride, and 1,2-dichloroethane; nitriles such as acetonitrile and propionitrile; acetic anhydride such as acid anhydride; and mixtures thereof.

The reaction temperature is generally in the range of about 10 minutes to about 48 hours, preferably about 30 minutes to about 24 hours.

The reaction temperature is generally in the range of about =20 to 150° C., preferably about 0 to 100° C.

The reaction time can be shortened using a microwave reactor or the like.

The compound (I₀) thus obtained may be isolated from the reaction mixture by a conventional method and can be easily purified by any of well-known separation techniques, such as concentration, vacuum concentration, solvent extraction, crystallization, transfer dissolution, and chromatography.

The compound (I₀) can be produced by the process represented by Reaction Formula 2 as follows.

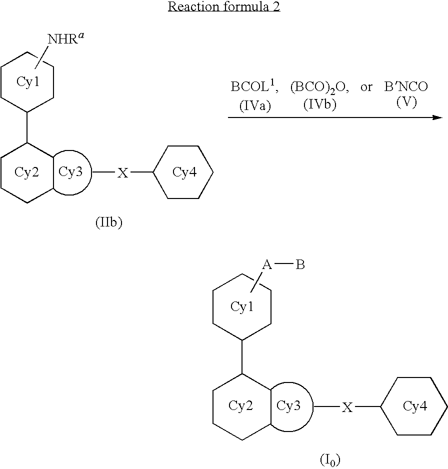

wherein B′ represents that, when B is an amino group which may be substituted, an amino group is removed from B; and other symbols are synonymous with those described above.

The compound (I₀) can be produced by reaction of the compound (IIb) with the compound (IVa), compound (IVb), or compound (V) in the presence of base or acid if required.

The compound (IVa), compound (IVb), or compound (V) may be any of commercially available products or may be produced according to a well-known method or another method based thereon.

The amount of each of the compound (IVa), compound (IVb), or compound (V) used is about 1 to 10 mol, preferably about 1 to 2 mol per mol of the compound (IIb).

Examples of the “base” include basic salts such as sodium carbonate, potassium carbonate, cesium carbonate, and sodium bicarbonate; aromatic amines such as pyridine and lutidine; tertiary amines such as triethylamine, diisopropylethylamine, tripropylamine, tributylamine, cyclohexyldimethylamine, 4-dimethylaminopyridine, N-methylpiperidine, N-methylpyrrolidine, and N-methylmorpholine; alkaline metal hydrides such as sodium hydride and potassium hydride; metal amides such as sodium amide, lithium diisopropyl amide, and lithium hexamethyidisilazide; and metal alkoxides such as sodium methoxide, sodium ethoxide, and sodium tert-butoxide.

The amount of the “base” used is generally about 0.1 to 10, preferably 0.8 to 2 equivalent per compound (IIb). Examples of the “acid” include methane sulfonic acid, p-toluene sulfonic acid, and camphor sulfonic acid.

The amount of the “acid” used is generally about 0.1 to 10, preferably 0.8 to 3 equivalent per compound (IIb).

It is advantageous to carry out the present reaction in the absence of a solvent or in the presence of a solvent inactive to the reaction. Preferable examples of such a solvent include, but not specifically limited as long the reaction proceeds, water; ethers such as diethyl ether, tetrahydrofuran, 1,4-dioxane, and 1,2-dimethoxy ethane; hydrocarbons such as benzene, toluene, cyclohexane, and hexane; amides such as N,N-dimethyl formamide and N,N-dimethyl acetamide; halogenated hydrocarbons such as dichloromethane, chloroform, carbon tetrachloride, and 1,2-dichloroethane; nitriles such as acetonitrile and propionitrile; sulfoxides such as dimethyl sulfoxide; and nitrogen-containing aromatic hydrocarbons such as pyridine, lutidine, and quinoline, or mixtures thereof.

The reaction temperature is generally in the range of −40 to 150° C., preferably 0 to 110° C.

The reaction time is generally in the range of 5 minutes to 24 hours, preferably 10 minutes to 5 hours.

As an alternative method, the compound (II) may be reacted with BCOOH in the presence of an appropriate condensation agent.

The amount of the BCOOH used is generally about 0.8 to 10 mol, preferably about 0.8 to 2 mol per mol of the compound (IIb).

Examples of the “condensation agent” include: N,N′-carbodiimides such as N,N′-dicyclohexyl carbodiimide and 1-ethyl-3-(3-dimethylaminopropyl) carbodiimide hydrochloride salt (WSC); azorites such as N-N′-carbonylimidazole; 2-halogeno pyridinium salts such as 2-chloro-1-methyl pyridinium iodide and 2-fluoro-1-methyl pyridinium iodide; and other compounds such as N-ethoxycarbonyl-2-ethoxy-1,2-dihydroquinoline, diethylcyanophosphate, phosphorous oxychloride, and acetic anhydride.

The amount of the “condensation agent” used is generally about 0.8 to 5 mol, preferably about 1 to 3 mol per compound (IIb).

The reaction may be carried out in the presence of a base if required. Examples of the “base” include basic salts such as potassium acetate and sodium acetate; tertiary amines such as triethylamine, diisopropylethylamine, tripropylamine, tributylamine, cyclohexyl dimethylamine, 4-dimethylaminopyridine, N-methylpiperidine, N-methylpyrrolidine, and N-methylmorpholine. In addition, if required, the reaction may be carried out in the presence of a condensation accelerator such as 1-hydroxy-1H-benzotriazole (HOBt)monohydrate or the like.

The amount of “base” used is generally about 0.5 to 5 mol, preferably about 2 to 3 mol per mol of the compound (IIb).

It is advantageous to carry out the present reaction using a solvent inactive to the reaction. Preferably, examples of such a solvent include: alcohols such as methanol, ethanol, and propanol; hydrocarbons such as hexane, cyclohexane, benzene, toluene, and xylene; ethers such as diethyl ether, diisopropyl ether, tetrahydrofuran, 1,4-dioxane, and 1,2-dimethoxy ethane; amides such as N,N-dimethyl formamide, N,N-dimethyl acetamide, hexamethyl phosphoric triamide, and 1-methyl pyrrolidine-2-one; sulfoxides such as dimethyl sulfoxide; halogenated hydrocarbons such as dichloromethane, chloroform, carbon tetrachloride, and 1,2-dichloroethane; nitriles such as acetonitrile and propionitrile; acid anhydride such as acetic anhydride; and mixtures thereof.

The reaction time is generally in the range of about 10 minutes to about 48 hours, preferably about 30 minutes to about 24 hours.

The reaction temperature is generally in the range of about −20 to 150° C., preferably about 0 to 100° C.

The reaction time can be shortened using a microwave reactor or the like.

The compound (I₀) thus obtained may be isolated from the reaction mixture by a conventional method, and can be easily purified by any of well-known separation techniques, such as concentration, vacuum concentration, solvent extraction, crystallization, transfer dissolution, and chromatography.

The compound (I₀) in which B is —NHB′ can be also produced by the process represented by Reaction Formula 3 below. In other words, the compound (IIb) can be 2,2,2-trichloroethoxycarbonylated with 2,2,2-trichloroethyl chloroformate to prepare compound (I′). Subsequently, the compound (I′) is reacted with compound (VI), thereby obtaining the compound (I₀).

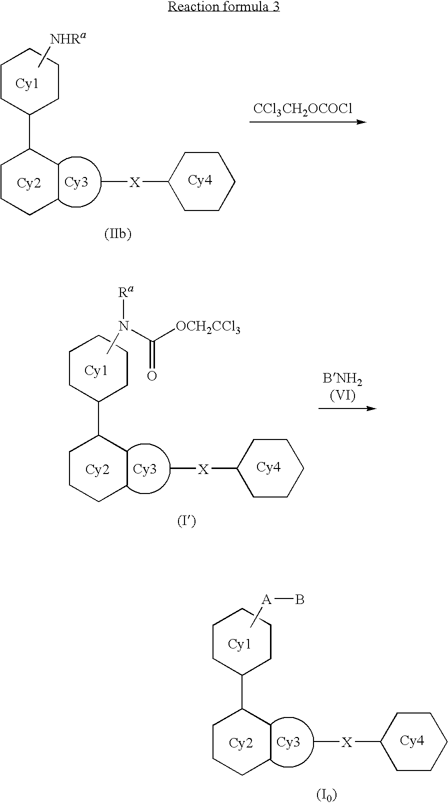

In the reaction formula, symbols are synonymous with those described above, respectively.

The compound (I′) can be produced from the compound (IIb) in a manner similar to the production of the compound (I₀) from the compound (IIb).

The compound (I₀) can be produced by reaction of the compound (I′) with the compound (VI) in a solvent that does not affect on the reaction under basic conditions.

The compound (VI) may be a commercially available products or may be produced according to a well-known method or another method based thereon.

The amount of the compound (VI) used is generally about 2 to 10 mol, preferably about 2 to 5 mol per mol of the compound (I′).

The examples of the “base” include pyridine, triethylamine, diisopropylethylamine, potassium carbonate, sodium carbonate, sodium hydride, and potassium hydride.

The amount of the “base” used is generally about 2 to 10 mole, preferably about 2 to 5 mol per mol of the compound (I′).

Examples of the solvent that does not affect on the reaction include: ethers such as tetrahydrofuran; halogenated hydrocarbons such as chloroform; aromatic hydrocarbons such as toluene; amides such as N,N-dimethyl formamide; and sulfoxides such as dimethyl sulfoxide. Two or more of these solvents may be mixed together at a suitable ratio.

The reaction temperature is generally in the range of about −50 to 200° C., preferably about 0 to 100° C.

The reaction time is generally in the range of about 10 minutes to about 36 hours, preferably about 30 minutes to about 24 hours.

The compound (I₀) thus obtained may be isolated from the reaction mixture by a conventional method, and can be easily purified by any of well-known separation techniques, such as concentration, vacuum concentration, solvent extraction, crystallization, transfer dissolution, and chromatography.

The compound (I₀) can be produced by the process represented by Reaction Formula 4 as follows.

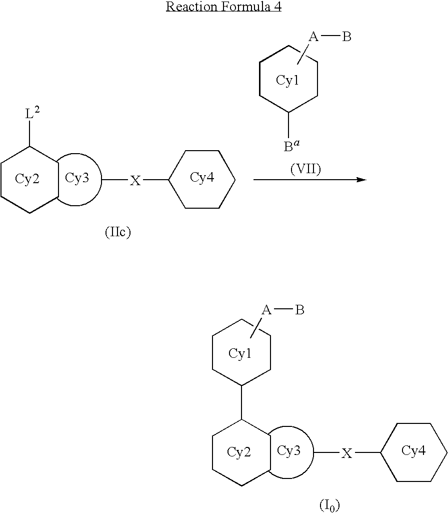

In the reaction formula, L² represents a leaving group; B^{a} represents B(OR^{c})₂(wherein “R^{c}”s represent, a C₁₋₆ alkyl group or two “R^{c}”s may be combined together to form a C₂₋₆ alkylene chain); and other symbols are synonymous with those described above).

Examples of the C₂₋₆ alkylene chain formed by combining two R^{C}s with each other include —CH₂—CH₂—, —C(CH₃)₂—C(CH₃)₂—, —CH₂—CH₂—CH₂—, and —CH₂—C(CH₃)₂-CH₂—.

The compound (I₀) is produced by carrying out Suzuki coupling between the compound (IIc) and the compound (VII).

The reaction is carried out by reaction of the compound (IIc) with boronic acid (VII) in a solvent under basic conditions in the presence of a transition metal catalyst.

The compound (VII) may be any of commercially available products or may be produced according to a well-known method or another method based thereon.

Examples of the “leaving group” represented by L² include a halogen atom (e.g. chlorine, bromine, iodine), C₁₋₆ alkylsulfonyloxy group (e.g., trifluoromethane sulfonyloxy, methane sulfonyloxy) which may be halogenated.

Examples of the functional group represented by B(OR^{c})₂ include boronic acids and boronic esters (e.g., 4,4,5,5-tetramethyl-1,3,2-dioxaborolan-2-yl).

The amount of the “boronic acids” used is about 0.5 to 10 mol, preferably about 0.9 to 3 mol per mol of the compound (IIc).

Examples of the “base” include basic salts such as sodium carbonate, potassium carbonate, cesium carbonate, and sodium hydrogen carbonate; aromatic amines such as the pyridine, lutidine; tertiary amines such as triethyl amine, diisopropylethylamine, tripropyl amine, tributyl amine, cyclohexyl dimethyl amine, 4-dimethylaminopyridine, N,N-dimethyl aniline, N-methylpiperidine, N-methylpyrrolidine, and N-methylmorpholine; and metal alkoxides such as sodium methoxide, sodium ethoxide, sodium tert-butoxide, and potassium tert-butoxide.

Examples of the “transition metal catalyst” include palladium catalysts such as palladium acetate, palladium chloride, tetrakis(triphenylphosphine)palladium,

1,1 -bis-(diphenylphosphino)ferrocene dichloropalladium, and dichlorobis(triphenylphosphine)palladium. The amount of the transition metal catalyst used is about 0.001 to 3 mol, preferably about 0.02 to 0.2 mol per mol of the compound (IIc).

Examples of the solvent include: ethers such as diethyl ether, diisopropyl ether, tetrahydrofuran, dioxane, 1,2-dimethoxy ethane; alcohols such as methanol, ethanol, and propanol; hydrocarbons such as benzene, toluene, carbon disulfide, cyclohexane, and hexane; amides such as N,N-dimethyl formamide and N,N-dimethylacetamide; halogenated hydrocarbons such as dichloromethane, chloroform, carbon tetrachloride, and 1,2-dichloroethane; nitriles such as acetonitrile and propionitrile; sulfoxides such as dimethyl sulfoxide; and water or mixture solvents thereof.

The reaction temperature is generally in the range of 0 to 250° C., preferably 50 to 150° C. The reaction time is generally about 5 minutes to about 48 hours, preferably about 30 minutes to about 24 hours.

The reaction time can be shortened using a microwave reactor or the like.

In addition, compounds obtained in the respective steps may be directly used as a reaction solution or a crude product in the subsequent reaction. Alternatively, it may be isolated from the reaction mixture by a conventional method, and can be easily purified by any of well-known separation techniques, such as concentration, vacuum concentration, solvent extraction, crystallization, transfer dissolution, and chromatography.

The compound (I₀) can be produced by the process represented by Reaction Formula 5 as follows.

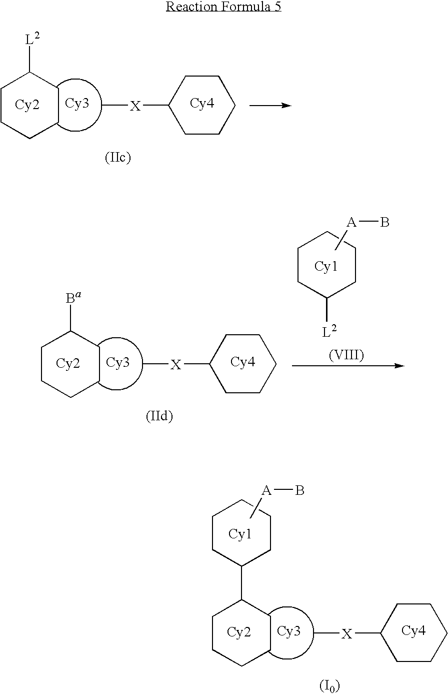

(In the reaction formula, symbols are synonymous with those described above, respectively)

The compound (IId) may be produced from the compound (IIc) according to a well-known method or another method based thereon.

The compound (I₀) can be produced from the compound (IId) and the compound (VIII) in a manner similar to the production of the compound (I₀) from the compound (IIc) as described in Reaction Formula 4.

The compound (VIII) may be any of commercially available products or may be produced according to a well-known method or another method based thereon.

The product may be isolated from the reaction mixture by a conventional method, and can be easily purified by any of well-known separation techniques, such as concentration, vacuum concentration, solvent extraction, crystallization, transfer dissolution, and chromatography.

The compound (I₀) can be produced by the method described in Reaction Formula 6 as described below when the Cy3 ring contains (—NH—) and X denotes alkylene.

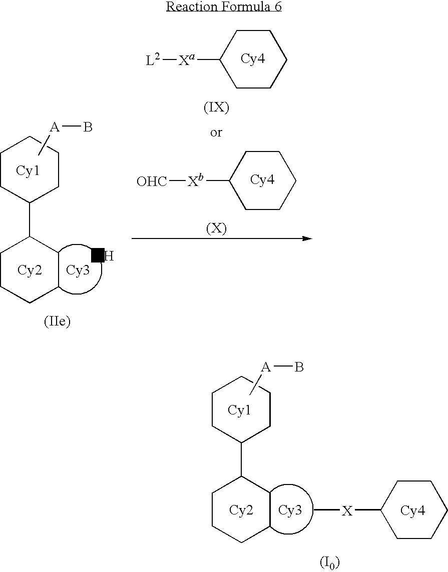

In the reaction formula, X^{a} represents —CH₂— or —(CH₂)₂—. X^{b} represents a bond or —CH₂—. Other symbols are synonymous with those described above, respectively.

The compound (I₀) can be produced by reaction of the compound (IIe) with the compound (IX) in the presence of a base if required.

The amount of the compound (IX) used is about 0.8 to 5.0 mol, preferably about 1.0 to 2.0 mol per mol of the compound (IIe).

Examples of the “base” include basic salts such as sodium carbonate, potassium carbonate, cesium carbonate, and sodium hydrogen carbonate; aromatic amines such as the pyridine, lutidine; tertiary amines such as triethyl amine, diisopropylethylamine, tripropyl amine, tributyl amine, cyclohexyl dimethyl amine, 4-dimethylaminopyridine, N,N-dimethylaniline, N-methylpiperidine, N-methylpyrrolidine, and N-methylmorpholine; alkali metal hydrides such as sodium hydride and potassium hydride; metal amides such as sodium amide, lithium diisopropyl amide, and lithium hexamethyldisilazide; and metal alkoxides such as sodium methoxide, sodium ethoxide, sodium tert-butoxide, and potassium tert-butoxide.

The amount of the base used is about 0.8 to 5.0 mol, preferably about 1.0 to 2.0 mol per mol of the compound (IIe).

It is advantageous to carry out the present reaction in the presence of a solvent inactive to the reaction. Preferable examples of such a solvent include, but not specifically limited as long the reaction proceeds, alcohols such as methanol, ethanol, and propanol; ethers such as diethyl ether, tetrahydrofuran, dioxane, and 1,2-dimethoxy ethane; hydrocarbons such as benzene, toluene, cyclohexane, and hexane; amides such as N,N-dimethyl formamide and N,N-dimethyl acetamide; halogenated hydrocarbons such as dichloromethane, chloroform, carbon tetrachloride, and 1,2-dichloroethane; nitriles such as acetonitrile and propionitrile; and sulfoxides such as dimethyl sulfoxide; or mixture solvents thereof.

The reaction time is generally about 30 minutes to about 48 hours, preferably about one hour to about 24 hours. The reaction temperature is generally about −20 to 200° C., preferably about 0 to 150° C.

Furthermore, instead of the above reaction, the compound (I₀) may be prepared using the compound (IIe) and the compound (X) by a reductive amination reaction using a reductant.

The compound (X) may be any of commercially available products or may be produced according to a well-known method or another method based thereon.

The amount of the compound (X) used is about 0.8 to 5.0 mol, preferably 1.0 to 2.0 mol per mol of the compound (IIe).

Examples of the “reduction agent” include: metal hydrides such as sodium borohydride, sodium cyanoborohydride, sodium triacetoxyborohydride, and lithium aluminum hydride; boranes such as a borane-tetrahydrofuran complex; hydrosilanes such as triethyl silane; or formic acid. If desired, an acid catalyst may be added together with the reductant. Examples of the acid catalyst include: mineral acids such as hydrochloric acid, hydrobromic acid, and sulfuric acid; sulfonic acids such as methane sulfonic acid and p-toluene sulfonic acid; organic acids such as acetic acid, propionic acid, and trifluoroacetic acid; and Lewis acids such as zinc chloride and aluminum chloride.

The amount of the “reductant” used is about 0.25 to 5.0 mol, preferably about 0.5 to 2.0 mol.

The amount of the acid catalyst used is, for example in the case of mineral acids, generally about 1 to 100 mol, preferably about 1 to 20 mol per mol of the compound (IIe).

It is advantageous to carry out the present reaction in the presence of a solvent inactive to the reaction. Preferable examples of such a solvent include, but not specifically limited as long the reaction proceeds, alcohols such as methanol, ethanol, and propanol; ethers such as diethyl ether, tetrahydrofuran, dioxane, and 1,2-dimethoxy ethane; hydrocarbons such as benzene, toluene, cyclohexane; amides such as N,N-dimethyl formamide and N,N-dimethyl acetamide; and mixture solvents thereof.

The reaction time is generally about 5 minutes to about 48 hours, preferably about 30 minutes to about 24 hours.

The reaction temperature is generally about −20 to 200° C., preferably about 0 to 100° C.

After condensation of the compound (IIe) and the compound (X), instead of reduction with a reductant, the compound (I₀) may be also produced by a catalytic hydrogenation reaction with any of various catalysts under hydrogen atmosphere.

Examples of the catalyst used include platinum oxide, platinum activated carbon, palladium activated carbon, nickel, copper-chromium oxide, rhodium, cobalt, and ruthenium. The amount of the catalyst used is about 1 to 1000% by weight, preferably about 5 to 50% by weight with respect to the compound (IIe).

It is advantageous to carry out the present reaction in the presence of a solvent inactive to the reaction. Preferable examples of such a solvent include, but not specifically limited as long the reaction proceeds, alcohols such as methanol, ethanol, and propanol; ethers such as diethyl ether, tetrahydrofuran, dioxane, and 1,2-dimethoxy ethane; hydrocarbons such as benzene, toluene, cyclohexane, and hexane; amides such as N,N-dimethyl formamide and N,N-dimethyl acetamide; water; and mixture solvents thereof.

The reaction time is generally about 30 minutes to about 48 hours, preferably about 30 minutes to about 24 hours. The reaction temperature is generally about 0 to 120° C., preferably about 20 to 80° C.

In addition, the product may be directly used as a reaction solution or a crude product in the subsequent reaction. Alternatively, the product may be isolated from the reaction mixture by a conventional method, and can be easily purified by any of well-known separation techniques, such as concentration, vacuum concentration, solvent extraction, crystallization, transfer dissolution, and chromatography.

The compound (I₀) can be produced by the process represented by Reaction Formula 7 as follows.

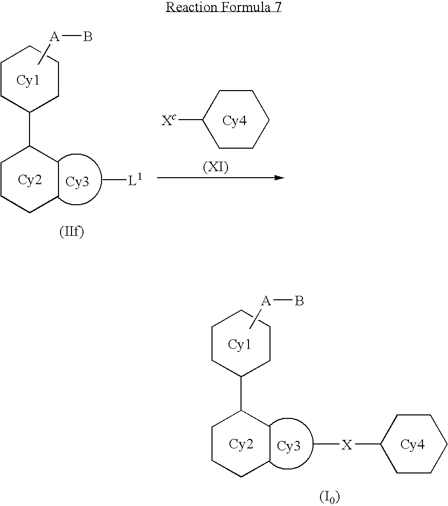

In the reaction formula, X^{c} represents a substituent selected from a hydroxy group, an amino group and a mercapto group, and other symbols are synonymous with those described above, respectively.

The compound (I₀) can be produced by reaction of the compound (IIf) with the compound (XI) in the presence of a base if required.

The compound (XI) may be any of commercially available products or may be produced according to a well-known method or another method based thereon.

The amount of the compound (XI) used is about 0.8 to 5.0 mol, preferably about 1.0 to 2.0 mol per mole of the compound (IIf).

Examples of the “base” include: basic salts such as sodium carbonate, potassium carbonate, cesium carbonate, and sodium hydrogen carbonate; aromatic amines such as the pyridine and lutidine; tertiary amines such as triethyl amine, diisopropylethylamine, tripropyl amine, tributyl amine, cyclohexyl dimethyl amine, 4-dimethylaminopyridine, N,N-dimethylaniline, N-methylpiperidine, N-methylpyrrolidine, and N-methylmorpholine; alkali metal hydrides such as sodium hydride and potassium hydride; metal amides such as sodium amide, lithium diisopropyl amide, and lithium hexamethyldisilazide; and metal alkoxides such as sodium methoxide, sodium ethoxide, sodium tert-butoxide, and potassium tert-butoxide.

The amount of the base used is about 0.8 to 5.0 mol, preferably about 1.0 to 2.0 mol per mol of the compound (IIf). It is advantageous to carry out the present reaction in the presence of a solvent inactive to the reaction. Preferable examples of such a solvent include, but not specifically limited as long the reaction proceeds, alcohols such as methanol, ethanol, and propanol, ethers such as diethyl ether, tetrahydrofuran, dioxane, and 1,2-dimethoxy ethane; hydrocarbons such as benzene, toluene, cyclohexane, and hexane; amides such as N,N-dimethyl formamide and N,N-dimethyl acetamide; halogenated hydrocarbons such as dichloromethane, chloroform, carbon tetrachloride, and 1,2-dichloroethane; nitriles such as acetonitrile and propionitrile; sulfoxides such as dimethyl sulfoxide; and mixture solvents thereof.

The reaction time is generally about 30 minutes to about 48 hours, preferably about one hour to about 24 hours. The reaction temperature is generally about −20 to 200° C., preferably about 0 to 150° C.

Instead of the above reaction, the Mitsunobu reaction (“Synthesis”, pages 1-27, 1981) may be used.

The reaction of the compound (IIf) with a compound which is the compound (XI) in which L¹ and X^{c} are OH in the presence of azodicarboylates (e.g., diethyl azodicarboxylate) and phosphines (e.g., triphenyl phosphine and tributyl phosphine).

The amount of the compound (XI) used is about 1.0 to 5.0 mol, preferably about 1.0 to 2.0 mol per mol of the compound (IIf).

The amounts of “azodicarboylates” and “phosphines” used are about 1.0 to 5.0 mol, preferably about 1.0 to 2.0 mol per mol of compound (IIe), respectively.

It is advantageous to carry out the present reaction in the presence of a solvent inactive to the reaction.

Preferable examples of such a solvent include, but not specifically limited as long the reaction proceeds, ethers such as diethyl ether, tetrahydrofuran, dioxane, and 1,2-dimethoxy ethane; hydrocarbons such as benzene, toluene, cyclohexane, and hexane; amides such as N,N-dimethyl formamide and N,N-dimethyl acetamide; halogenated hydrocarbons such as dichloromethane, chloroform, carbon tetrachloride, and 1,2-dichloroethane; nitrites such as acetonitrile and propionitrile; sulfoxides such as dimethyl sulfoxide; and mixture solvents thereof.

The reaction time is generally about 5 minutes to about 48 hours, preferably about 30 minutes to about 24 hours. The reaction temperature is generally about −20 to 200° C., preferably about 0 to 100° C.

In addition, the product may be directly used as a reaction solution or a crude product in the subsequent reaction. Alternatively, the product may be isolated from the reaction mixture by a conventional method, and can be easily purified by any of well-known separation techniques, such as concentration, vacuum concentration, solvent extraction, crystallization, transfer dissolution, and chromatography.

The compound (IIa) can be produced by the process represented by Reaction Formula 8 as follows.

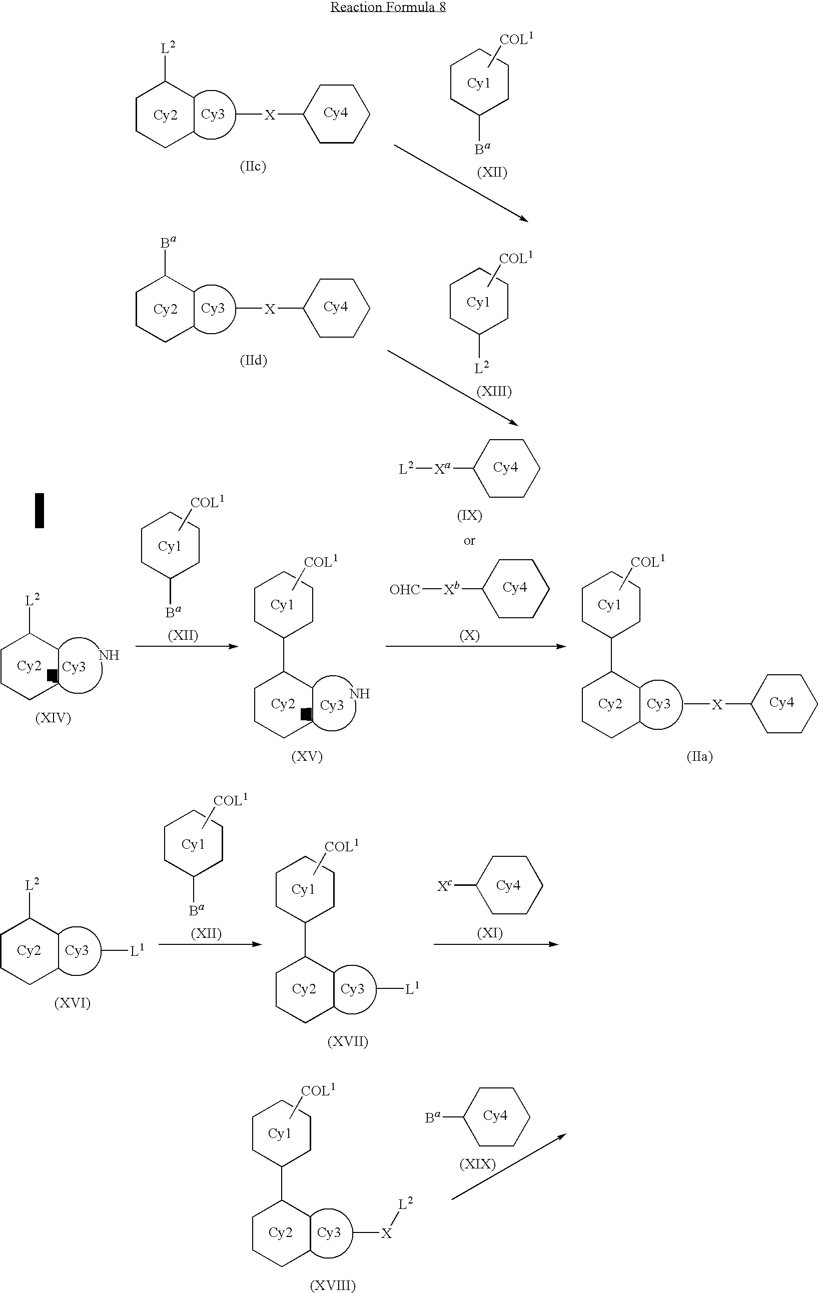

The symbols of the compounds in the reaction formula are synonymous with those described above.

The compound (IIa) can be produced from the compound (IIc) and the compound (XII) in a manner similar to the production of the compound (I₀) from the compound (IIc) as described in Reaction Formula 4; from the compound (IId) and the compound (XIII) in a manner similar to the production of the compound (I₀) from the compound (IIc) as described in Reaction Formula 4; from the compound (XV) in a manner similar to the production of the compound (I₀) from the compound (IIe) as described in Reaction Formula 6; from the compound (XVII) in a manner similar to the production of the compound (I₀) from the compound (IIf) as described in Reaction Formula 7; or from the compound (XVIII) in a manner similar to the production of the compound (I₀) from the compound (IIc) as described in Reaction Formula 4.

The compound (XV) and the compound (XVII) used as raw materials of the compound (IIa) can be produced respectively from the compound (XIV) and the compound (XVI) in a manner similar to the production of the compound (I₀) from the compound (IIc) as described in Reaction Formula 4.

The compound (XII) and the compound (XIX) may be any of commercially available products or may be produced according to a well-known method or another method based thereon.

The compound (IIa) thus obtained may be isolated from the reaction mixture by a conventional method, and can be easily purified by any of well-known separation techniques, such as concentration, vacuum concentration, solvent extraction, crystallization, transfer dissolution, and chromatography.

The compound (IIe) can be produced by the process represented by Reaction Formula 9 as follows.

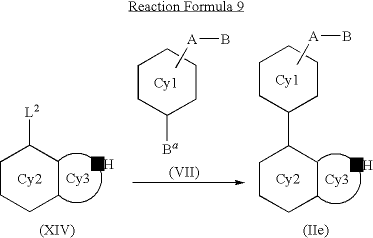

In the reaction formula, symbols are synonymous with those described above, respectively.

The compound (IIe) can be produced from the compound (XIV) in a manner similar to the production of the compound (I₀) from the compound (IIc) as described above in Reaction Formula 4.

The compound (XIV) may be commercially available products or may be produced according to a well-known method or another method based thereon.

The compound (IIe) thus obtained may be isolated from the reaction mixture by a conventional method, and can be easily purified by any of well-known separation techniques, such as concentration, vacuum concentration, solvent extraction, crystallization, transfer dissolution, and chromatography.

The compound (IIf) can be produced from the compound (XVI) by the process represented by Reaction Formula 10 as follows.

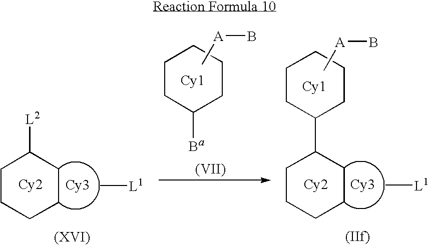

In the reaction formula, symbols are synonymous with those described above, respectively.

The compound (IIf) can be produced from the compound (XVI) in a manner similar to the production of the compound (I₀) from the compound (IIc) as described in Reaction Formula 4.

The compound (XVI) may be commercially available products or may be produced according to a well-known method or another method based thereon.

The compound (IIf) thus produced may be isolated from the reaction mixture by a conventional method, and can be easily purified by any of well-known separation techniques, concentration, vacuum concentration, solvent extraction, crystallization, transfer dissolution, and chromatography.

The compound (IIc) may be produced according to a well-known method or another method based thereon.

Alternatively, the compound (IIc) may be produced by the process represented by Reaction Formula 11 as follows.

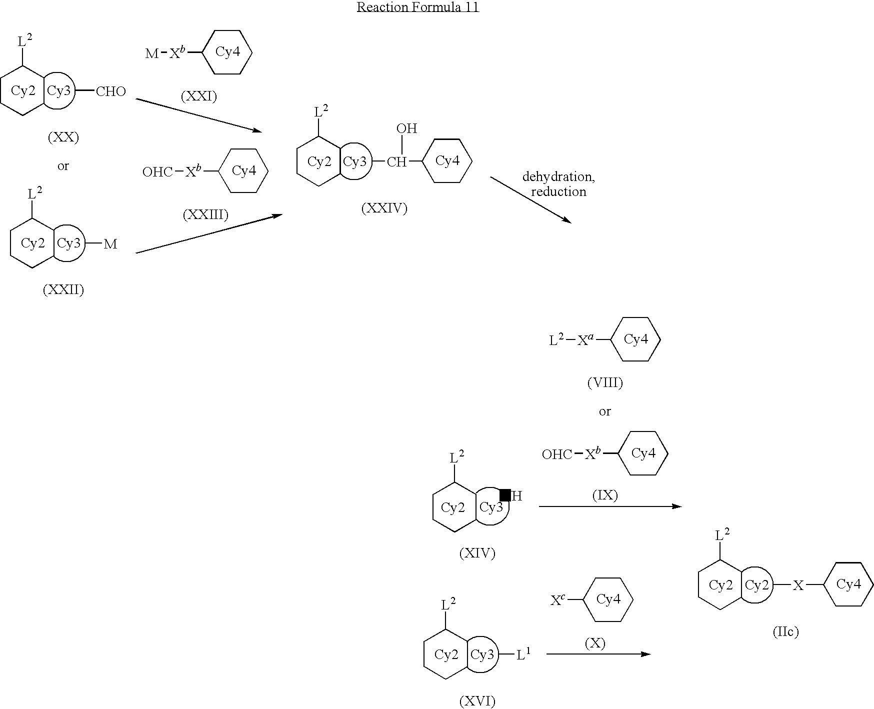

In the reaction formula; M represents MgL³ or Li, L³ represents halogen (e.g., chlorine, bromine, or iodine) and other symbols are synonymous with those described above, respectively.

The compound (XXIV) can be produced by reaction of the compound (XX) with the Grignard reagent or an organic lithium reagent (XXI) or by reaction of the compound (XXIII) with the Grignard reagent or an organic lithium reagent (XXII).

In the formula, the compound (XX) or the compound (XXIII) may be any of commercially available products or may be produced according to a well-known method or another method based thereon.

In the formula, the Grignard reagent or the organic lithium reagent (XXI or XXII) can be easily obtained as a commercial product or may be produced according to a well-known method or another method based thereon, such as one described in The Fourth Series of Experimental Chemistry, vol. 25 (Ed. Chemical Society of Japan), published by Maruzen Co., Ltd.

The amount of the Grignard reagent or the organic lithium reagent (XXI or XXII) used is about 0.8 to 30 mol, preferably about 1.0 to 20 mol per mol of the compound (XX) or the compound (XXIII).

In the present reaction, it is advantageous to carry out the present reaction in the absence of a solvent or in the presence of a solvent inactive to the reaction. Preferable examples of such a solvent include, but not specifically limited as long the reaction proceeds, alcohols such as methanol, ethanol, and propanol; hydrocarbons such as hexane, cyclohexane, benzene, toluene, and xylene; ethers such as diethyl ether, diisopropyl ether, tetrahydrofuran, dioxane, and 1,2-dimethoxy ethane; amides such as N,N-dimethyl formamide, N,N-dimethylacetamide, and hexamethyl phosphoric triamide; sulfoxides such as dimethyl sulfoxide; halogenated hydrocarbons such as dichloromethane, chloroform, carbon tetrachloride, and 1,2-dichloroethane; mixture solvents thereof.

The reaction time is generally about 10 minutes to about 24 hours, preferably about 30 minutes to about 12 hours. The reaction temperature is generally about −100 to 120° C., preferably about −80 to 60° C.

In addition, the product may be directly used as a reaction solution or a crude product in the subsequent reaction. Alternatively, the product may be isolated from the reaction mixture by a conventional method, and can be easily purified by any of well-known separation techniques, such as concentration, vacuum concentration, solvent extraction, crystallization, transfer dissolution, and chromatography.

Furthermore, the compound (IIc) can be produced by subjecting the compound (XXIV) to reductive dehydration.

The reductive dehydration may be carried out by a catalytic reduction method, a method using an organic silyl reagent (e.g., alkyl silane reagent), or the like.

In the catalytic reduction method, the compound (IIc) can be obtained by reaction of the compound (XXIV) with a metal catalyst under hydrogen atmosphere. The reaction may be carried out in the presence of an appropriate metal catalyst if required.

Examples of the “metal catalyst” include Raney nickel, platinum oxide, metal palladium, and palladium activated carbon. The amount of the “metal catalyst” used is generally about 1 to 1000% by weight, preferably about 5 to 20% by weight with respect to the compound (XXIV).

Examples of the “acid catalyst” include organic acids such as formic acid, acetic acid, trifluoroacetic acid, p-toluene sulfonic acid; and mineral acid such as sulfuric acid, hydrochloric acid, and hydrobromic acid. The amount of the “acid catalyst” used is about 0.1 mol or an excess amount thereof per mol of the compound (XXIV).

It is advantageous to carry out the present reaction in the presence of a solvent inactive to the reaction. Preferable examples of such a solvent include, but not specifically limited as long the reaction proceeds, alcohols such as methanol, ethanol, and propanol; ethers such as diethyl ether, tetrahydrofuran, dioxane, and 1,2-dimethoxy ethane; hydrocarbons such as benzene, toluene, cyclohexane, and hexane; amides such as N,N-dimethyl formamide and N,N-dimethyl acetamide; organic acids such as acetic acid; water; and mixture solvents thereof. A hydrogen pressure is generally about I to 100 atm, preferably about 1 to 5 atm. The reaction hour is generally about 30 minutes to about 48 hours, preferably about one hour to about 24 hours. The reaction temperature is generally about 0 to 120° C., preferably about 20 to 80° C.

After removal of the catalyst, the product may be isolated from the reaction mixture by a conventional method, and can be easily purified by any of well-known separation techniques, such as concentration, vacuum concentration, solvent extraction, crystallization, transfer dissolution, and chromatography.

In the process using an organic silyl reagent (alkylsilane reagent), the compound (IIc) can be produced by reaction of the compound (XXIV) with the alkylsilane reagent and the acid.

Examples of the alkylsilane reagent include triethyl silane and phenyldimethyl silane. The amount of the “alkylsilane reagent” used is about 0.8 to 20 mol, preferably about 1 to 10 mol per mol of the compound (XXIV).

The acid used may be an organic acid such as trifluoroacetic acid. The amount of the acid used is about 0.1 to an excessive amount per mol of the compound (XXIV).

It is advantageous to carry out the present reaction in the absence of a solvent or in the presence of a solvent inactive to the reaction. Examples of such a solvent include, but not specifically limited as long the reaction proceeds, ethers such as diethyl ether, tetrahydrofuran, dioxane, and 1,2-dimethoxy ethane; hydrocarbons such as benzene, toluene, cyclohexane, and hexane; organic acids such as acetic acid, trifluoroacetic acid, and mixture solvents thereof. The product may be isolated from the reaction mixture by a conventional method, and can be easily purified by any of well-known separation techniques, such as concentration, vacuum concentration, solvent extraction, crystallization, transfer dissolution, and chromatography.

In addition, the compound (IIc) can be produced from the compound (XIV) by Reaction Formula 6 in a manner similar to the production of the compound (I₀) from the compound (IIe) represented by Reaction Formula 6.

Furthermore, the compound (IIc) can be produced from the compound (XVI) in a manner similar to the production of the compound (I₀) from the compound (IIf) represented by Reaction Formula 7.

The compound (XVIII) may be produced by the process represented by Reaction Formula 12 as follows.

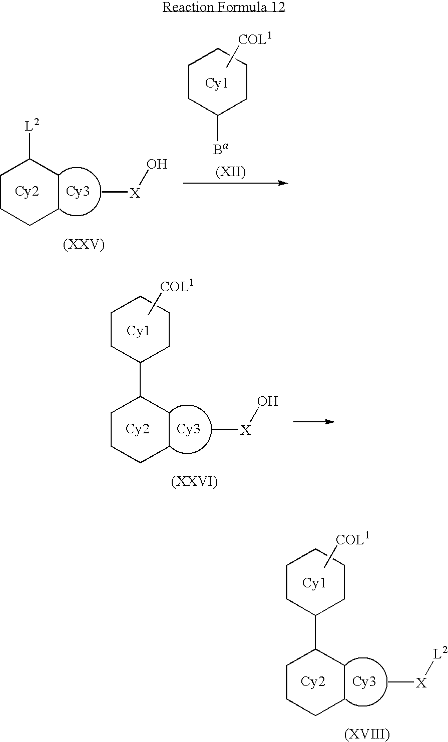

First, the compound (XXVI) is produced from the compound (XXV) and the compound (XII) in a manner similar to the production of the compound (I₀) from the compound (IIc) as described in Reaction Formula 4.

In the formula, the compound (XXV) may be any of commercially available products or may be produced according to a well-known method or another method based thereon. Subsequently, the compound (XXVI)is halogenated or converted into a C₁₋₆ alkylsulfonyloxy form which may be halogenated, thereby obtaining the compound (XXVI).

In the formula, the halogenation may be carried out by a well-known method, such as one described in The Fourth Series of Experimental Chemistry, vol. 19 (Ed. Chemical Society of Japan), published by Maruzen Co., Ltd.

The compound (XVIII) thus obtained may be isolated from the reaction mixture by a conventional method, and can be easily purified by any of well-known separation techniques, such as concentration, vacuum concentration, solvent extraction, crystallization, transfer dissolution, and chromatography.

The compound (IIb) can be produced by the process represented by Reaction Formula 13 as follows.

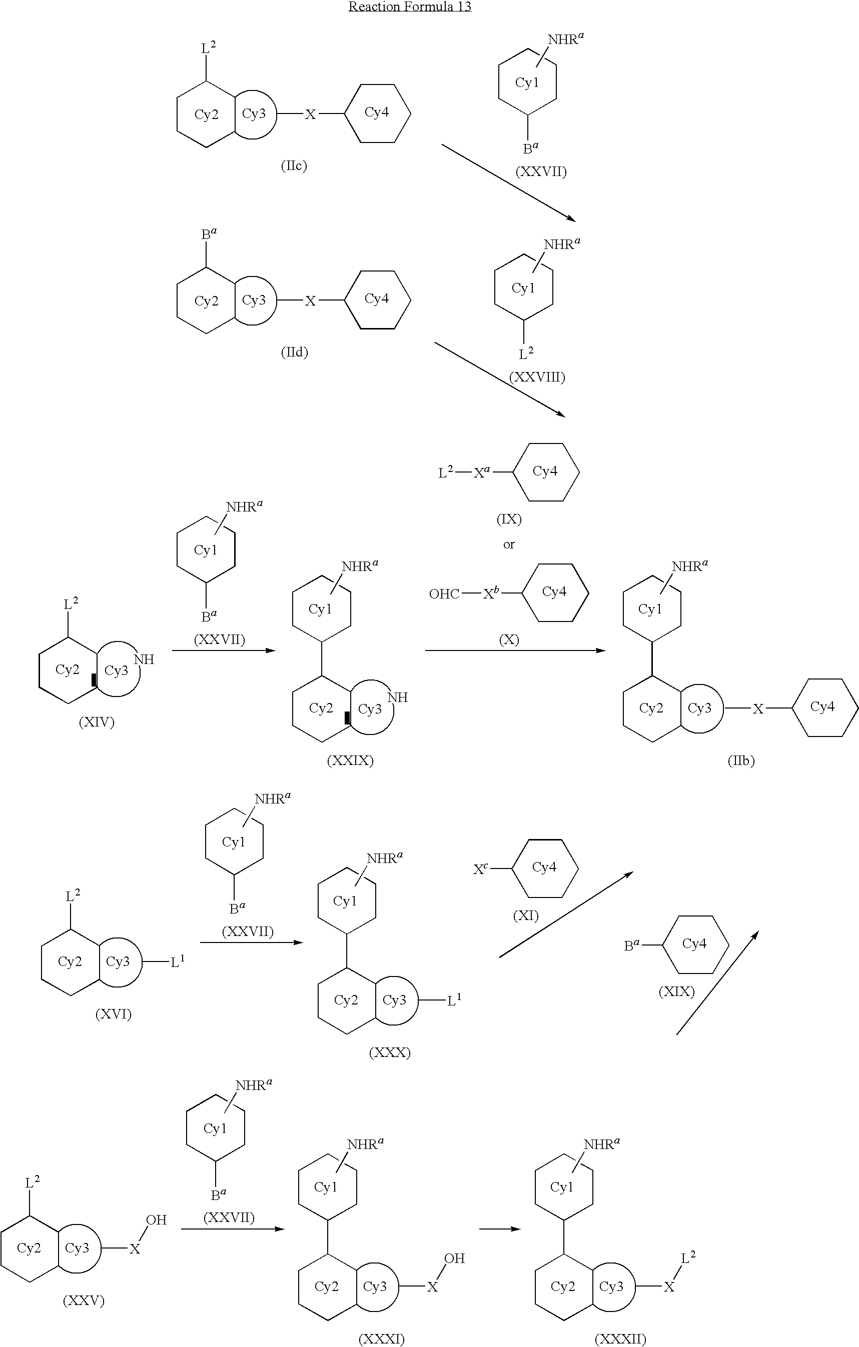

The compound (IIb) can be produced from the compound (IIc) and the compound (XXVII) in a manner similar to the production of the compound (I₀) from the compound (IIc) as described in Reaction Formula 4. Alternatively, the compound (IIb) can be produced from the compound (IId) and the compound (XXVIII) in a manner similar to the production of the compound (I₀) from the compound (IIc) as described in Reaction Formula 4. Alternatively, the compound (IIb) can be produced such that the compound (XXIX) is produced from the compound (XIV) in a manner similar to the production of the compound (I₀) from the compound (IIc) as described in Reaction Formula 4 and then processed in a manner similar to the production of the compound (I₀) from the compound (IIe) described in Reaction Formula 6. Alternatively, the compound (IIb) can be produced such that the compound (XXX) is produced from the compound (XVI) in a manner similar to the production of the compound (I₀) from the compound (IIc) as described in Reaction Formula 4 and then processed in a manner similar to the production of the compound (I₀) from the compound (IIf) described in Reaction Formula 7. Alternatively, the compound (IIb) can be produced such that the compound (XXXII) is produced from the compound (XXV) in a manner similar to the production of the compound (XVIII) from the compound (XXV) as described in Reaction Formula 12 and then processed in a manner similar to the production of the compound (I₀) from the compound (IIc) described in Reaction Formula 4.

The compound (IIb) thus obtained may be isolated from the reaction mixture by a conventional method, and can be easily purified by any of well-known separation techniques, such as concentration, vacuum concentration, solvent extraction, crystallization, transfer dissolution, and chromatography.

In each case, if required, the product is further subjected to one or any combination of well-known reactions, such as protection/deprotection, acylation, alkylation, hydrogenation, oxidation, reduction, carbon-chain extension, and substituent change. Consequently, the compound (I₀) can be synthesized.

In the case that the product of interest is obtained in free form, it may be converted into salt form by an ordinary method. If the product of interest is obtained in salt form, it may be converted into a free body or another salt by an ordinary method. The compound (I₀) thus obtained may be isolated and purified from a reaction solution by any of well-known techniques, such as transfer dissolution, concentration, solvent extraction, cracking, crystallization, recrystallization, and chromatography.

Furthermore, if the compound (I₀) is present as a configurational isomer, a diastereomer, or a conformer, it may be isolated by any of the separation and purification techniques if required. In addition, if the compound (I₀) is present as a racemic body, it can be separated into a d-isomer and an 1-isomer using a usual optical separation technique.

In addition to the compound (I₀), the product may be used as a prodrug of the compound (I₀). The prodrug of the compound (I₀) means a compound which can be converted into the compound (I₀) by reaction with oxygen, gastric acid, or the like under physiological conditions in the living body. In other words, it means a compound which can be converted into the compound (I₀) by hydrolysis with gastric acid or the like.

Examples of the prodrug of the compound (I₀) include a compound in which an amino group of the compound (I₀) is acylated, alkylated, or phosphorylated (e.g., the amino group of the compound (I₀) is eicosanoylated, alanylated, pentylaminocarbonylated, (5-methyl-2-oxo-1,3-dioxolen-4-yl) methoxycarbonylated, tetrahydrofuranylated, pyrrolidylmethylated, pivaloyloxymethylated, or tert-butylated); a compound in which a hydroxy group of the compound (I₀) is acylated, alkylated, phosphorylated, or borated (e.g., the hydroxy group of the compound (I₀) is acetylated, palmitoylated, propanoylated, pivaloylated, succinylated, fumarylated, alanylated, or dimethylaminomethylcarbonylated); a compound in which a carboxyl group of compound (I₀) is esterified or amidated (e.g., a compound in which a carboxyl group of the compound (I₀) is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified, or methylamidated). These compounds can be produced from compound (I₀) by a well-known method. The prodrug of compound (I₀) may be a compound that converts to the compound (I₀) under physiological conditions as described in Development of Pharmaceutical Products, vol. 7, Molecule Design, 163-198, Hirokawa Shoten (1990).

The compound of the present invention has an excellent GPR52 agonist activity and can be used as a preventive or therapeutic agent to mammals (e.g., humans, cows, horses, dogs, cats, mice, and rats, particularly humans among them) for diseases, such as mental diseases (e.g., mental diseases (e.g., schizophrenia, depression, anxiety, bipolar disorder or PTSD, aporioneurosis, and obsessive-compulsive disorder); neurodegenerative diseases (e.g., Alzheimer's disease, mild cognitive impairment (MCI), and Parkinson's disease); amyotrophic lateral sclerosis (ALS), Huntington's disease; spinocerebellar degeneration; multiple sclerosis (MS); and Pick disease. In particular, the compound of the present invention is useful for improving the medical conditions of schizophrenia, such as (1) positive symptoms such as delusions and hallucination; (2) negative symptoms such as hypesthesia, social withdrawal, and disinclination or loss of concentration; and (3) cognitive function disorders.

The compound of the present invention is superior in metabolic stability, so that the compound of this invention can be expected to have an excellent therapeutic effect on the above diseases even in a small dose.

The compound of the present invention has low toxicity (which is a pharmaceutical agent superior to others with respect to, for example, acute toxicity, chronic toxicity, genotoxic property, genotoxicity, cardiotoxicity, drug interactions, and carcinogenicity). The compound of the present invention is directly used as a pharmaceutical agent or a pharmaceutical composition mixed with a pharmaceutically accepted carrier or the like to be orally or parenterally administered to mammals (e.g., humans, monkeys, cows, horses, pigs, mice, rats, hamsters, rabbits, cats, sheep, and goats)) in safety. The term “parenterally” means intravenous, intramuscular, subcutis, intraorgan, intranasal, intracutaneous, eye-drop, intracerebral, rectal, intravaginal, or intraabdominal administration.

The term “pharmaceutically acceptable carrier” means any of various organic or inorganic carriers conventionally used as materials for pharmaceutical preparations, which are added as excipient, lubricant, binder and disintegrant for solid preparations; and solvent, dissolution aids, suspending agent, isotonicity agent, buffer and soothing agent and the like for liquid preparations. Where necessary, preparation additive such as preservative, antioxidant, coloring agent, sweetening agent and the like can be used.

Preferable examples of the excipient include lactose, sucrose, D-mannitol, D-sorbitol, starch, pregelatinized starch, dextrin, crystalline cellulose, low-substituted hydroxypropyl cellulose, sodium carboxymethyl cellulose, gum arabic, pullulan, light anhydrous silicic acid, synthetic aluminum silicate, and magnesium aluminometasilicate.

Preferable examples of the lubricant include magnesium stearate, calcium stearate, talc, colloidal silica and the like.

Preferable examples of the binder include pregelatinized starch, saccharose, gelatin, gum arabic, methylcellulose, carboxymethyl cellulose, sodium carboxymethyl cellulose, crystalline cellulose, sucrose, D-mannitol, trehalose, dextrin, pullulan, hydroxypropyl cellulose, hydroxypropyl methyl cellulose, and polyvinyl pyrrolidone.

Preferable examples of the disintegrant include lactose, sucrose, starch, carboxymethyl cellulose, carboxymethyl cellulose calcium, croscarmellose sodium, carboxymethyl starch sodium, light anhydrous silicic acid, and low-substituted hydroxypropylcellulose.

Preferable examples of the solvent include water for injection, physiological saline, Ringer's solution, alcohol, propylene glycol, polyethylene glycol, sesame oil, corn oil, olive oil, and cottonseed oil.

Preferable examples of the dissolution aids include polyethylene glycol, propylene glycol, D-mannitol, trehalose, benzyl benzoate, ethanol, trisaminomethane, cholesterol, triethanolamine, sodium carbonate, sodium citrate, sodium salicylate, and sodium acetate.

Preferable examples of the suspending agent include surfactants such as stearyl triethanol amine, sodium lauryl sulfate, lauryl aminopropionate, lecithin, benzalkonium chloride, benzethonium chloride, and glycerol monostearate; for example, hydrophilic polymers such as polyvinyl alcohol, polyvinyl pyrrolidone, sodium carboxymethyl cellulose, methylcellulose, hydroxymethyl cellulose, hydroxyethyl cellulose, and hydroxypropyl cellulose; polysorbates, and polyoxyethylene hydrogenated castor oil.

Preferable examples of an isotonicity agent include sodium chloride, glycerin, D-mannitol, D-sorbitol, and glucose.

Preferable examples of the buffer include buffers such as phosphate, acetate, carbonate, and citrate.

Preferable examples of the soothing agent include benzyl alcohol.

Preferable examples of the preservative include p-hydroxybenzoic acid esters, chlorobutanol, benzyl alcohol, phenethyl alcohol, dehydroacetic acid, and sorbic acid.

Preferable examples of the antioxidant include sulfite, and ascorbate.

Preferable examples of the coloring agent include water-soluble edible tar pigments (e.g., food colors such as Food Color Red Nos. 2 and 3, Food Color Yellow Nos. 4 and 5, and Food Color Blue Nos. 1 and 2), water-insoluble lake pigments (e.g., aluminum salt of the aforementioned water-soluble edible tar pigment), and natural pigments (e.g., β-carotene, chlorophil, and colcothar).

Preferable examples of the sweetening agent include saccharin sodium, dipotassium glycyrrhizinate, aspartame, and stevia.

Examples of the dosage form of the agent of the present invention include oral agents such as tablets (inclusive of sugarcoated tablets, film-coating tablets, sublingual tablets, and orally disintegrable tablets), capsules (inclusive of soft capsules and micro capsules), granules, powders, troches, syrups, emulsions, suspensions, and films(e.g., film disintegrable in the mouth); and parenteral agents such as injections (e.g., subcutaneous injections, intravenous injections, intramuscular injections, intraperitoneal injections, and drip infusion), external agents (e.g., transdermal preparations and ointments), suppositories (e.g., rectal suppositories and vaginal suppositories), pellets, preparations for nasal administration, pulmonary preparations (inhalants), and eye drop. Any of these preparations can be can be safely administered orally or parenterally (e.g., locally, rectal, and intravenous administrations).

In addition, these preparations may also be controlled-release preparations such as rapid-release preparations and sustained-release preparations (e.g., sustained-release microcapsules etc.).

The pharmaceutical composition of the present invention can be produced by a conventional method in the technical field of drug formulation, for example, the method described in the Japan Pharmacopoeia and the like. Hereinafter, a method for preparing a pharmaceutical agent will be described in detail. The content of the compound of the present invention in the pharmaceutical composition of the present invention varies among formulations, the dosages of the compound of the present invention, and the like. For example, the content of the compound is about 0.01 to 100% by weight, preferably 0.1 to 95% by weight with respect to the total amount of the composition.

The dosage of the compound of the present invention varies among dosage subjects, routes of administration, subject diseases, symptoms, and the like. For example, when the compound is orally administered to a schizophrenia patient (adult, about 60 kg in weight), a normal single dosage of about 0.1 to 20 mg/kg weight, preferably about 0.2 to 10 mg/kg weight, more preferably about 0.5 to 10 mg/kg weight is preferably administered one or several times (e.g., three times) a day.

The compound of the present invention may be used in combination with any of other active components. Exemplary active components include:
atypical antipsychotic agents (e.g., clozapine, olanzapine, risperidone, aripiprazole, iloperidone, asenapine, ziprasidone, quetiapine, and zotepine);
typical antipsychotic agents (e.g., haloperidol and chlorpromazine);
selective serotonin reuptake inhibitors (e.g., paroxetine, sertraline, fluvoxamine, and fluoxetine);
selective serotonin-noradrenaline reuptake inhibitors (e.g., milnacipran and venlafaxine);
selective noradrenaline-dopamine reuptake inhibitors (e.g., bupropion);
tetracyclic antidepressants (e.g., amoxapine and clomipramine);
tricyclic antidepressants (e.g., imipramine and amitriptyline);
other antidepressant agents (e.g., NS-2359, Lu AA21004, and DOV21947);
α7-nicotinic receptor partial modifiers (e.g., SSR-180711 and PNU-120596);
NK2 antagonists;
NK3 antagonists;
glycine transporter 1 inhibitors (e.g., ALX5407 and SSR504734);
metabolic glutamate receptor modifiers (e.g., CDPPB and MPEP);
antianxiety agents (benzodiazepine-based agents (e.g., diazepam and etizolam) and serotonin 5-HT_{1A} agonists (e.g., tandospirone);
sleep inducing agents (benzodiazepine-based agents (e.g., estazolam and triazolam), non-benzodiazepine-based agents (e.g., zolpidem), and melatonin receptor agonists (e.g., ramelteon));
β-amyloid vaccines;
β-amyloid degrading enzymes and the like;
brain function activators (e.g., aniracetam and nicergoline);
antiparkinson agents (e.g., dopamine receptor agonists ((e.g., L-DOPA, bromocriptine, pergolide, talipexole, pramipexole, cabergoline, and amantadine),
mono-amine oxidase (MAO) inhibitors (e.g., deprenyl, selegiline, remacemide, and riluzole), anticholinergic agents (e.g., trihexyphenidyl and biperiden); and COMT inhibitors (e.g., entacapone));
therapeutic agents for amyotrophic lateral sclerosis (e.g., neurotrophic factors such as riluzole);
antihyperlipidemic drugs such as cholesterol-lowering drugs (statin-based agents (e.g., statins (e.g., pravastatin sodium, atorvastatin, simvastatin, and rosuvastatin), fibrates (e.g., clofibrate), and squalene synthase inhibitors); therapeutic agents for abnormal behaviors, wandering symptoms associated with dementia (e.g., a sedative and anxiolytic), and the like;
apoptosis inhibitors (e.g., CPI-1189, IDN-6556, and CEP-1347); neuronal differentiation/regeneration accelerator (e.g., leteprinim and xaliproden (SR-57746-A), and SB-216763);
antihypertensive agents;
therapeutic agents for diabetes mellitus;
nonsteroidal anti-inflammatory agents (e.g., meloxicam, tenoxicam, indomethacin, ibuprofen, celecoxib, rofecoxib, and aspirin);
disease modifying antirheumatic drugs (DMARDs);
anticytokine agents (e.g., TNF inhibitors and MAP kinase inhibitors);
steroid agents (e.g., dexamethasone, hexestrol, and cortisone acetate);
sex hormones or the derivatives thereof (e.g., progesterone, estradiol, and estradiol benzoate);
parathyroid hormone (PTH); and
calcium receptor blockers (hereinafter, also simply referred to as combination drugs).

In particular, the compound of the present invention can be preferably used in combination with any of various central nervous system drugs and therapeutic agents for diseases easily developed with schizophrenia (e.g., therapeutic agents for diabetes mellitus).

In particular, the compound of the present invention can be preferably used in combination with any of various active components that do not act on GPR52.

The dosage forms of the compound of the present invention and the combination drugs thereof are not specifically limited. Any dosage form may be employed as long as the compound of the present invention is combined with any of the combination drugs. Exemplary dosage forms include: (1) administration of a single pharmaceutical agent prepared by simultaneously formulating the compound of the present invention and the combination drug;

(2) simultaneous administration of two different pharmaceutical agents prepared by independently formulating the compound of the present invention and the combination drug;

(3) administration of two different pharmaceutical agents on the same administrating path at different times, which are independently formulated from the compound of the present invention and the combination drug;

(4) administration of two different pharmaceutical agents on different administrating paths at same time, which are independently formulated from the compound of the present invention and the combination drug;

(5) administration of two different pharmaceutical agents on different administrating paths at different times, which are independently formulated from the compound of the present invention and the combination drug (e.g., the compound of the present invention and the combination drug are administered in this order and vice versa); and the like. Hereinafter, these dosage forms are collectively referred to as a combination agent of the present invention.

When the combination agent of the present invention is administered, both the combination drug and the compound of the present invention may simultaneously administered. Alternatively, after the administration of a combination drug, the compound of the present invention may be administered. Alternatively, the combination drug may be administered after the administration of the compound of the present invention. In the case of administration at different times, the time difference may vary among effective components, dosage forms, and medication methods. For instance, there is a method in which, when the combination drug is administered first, the compound of the present invention is administered after one minute or more but not more than three days, preferably 10 minutes to one day, more preferably 15 minutes to one hour from the administration of the combination drug. For instance, there is another method in which, when the combination drug is administered after one minute or more but not more than one day, preferably 10 minutes to 6 hours, more preferably 15 minutes to one hour from the administration of the compound of the present invention.

The combination drug may be contained in any amount as long as a side effect does not pose a problem. The daily dose of the combination drug may vary depending on the target of administration, route of administration, diseases, and so on. For example, when orally administering to a schizophrenia patient (adult, about 60 kg in weight), it is desirable to administer the combination drug in general at a unit dose of abut 0.1 to 20 mg/kg weight, more preferably about 0.5 to 10 mg/kg weight. The unit dose of the combination drug may be preferably administered one to several times (e.g., three times) a day.

When the compound of the present invention is administered in combination with the combination drug, the amounts of the respective agents may be reduced within their safe ranges in consideration of their opposing effects.

The combination agent of the present invention is less toxic, so that it can be administered in safety in the form of a pharmaceutical composition prepared by mixing the compound of the present invention and/or the above combination drug with a pharmaceutically acceptable carrier according to a well-known method. Specifically, for example, it may be orally or parenterally administered in the form of a tablet (e.g., sugar-coated tablet or a film-coating tablet), powders, granules, capsules (inclusive of soft capsules), a liquid drug, an injection agent, a suppository agent, a sustained-release agent, or the like (e.g., locally, rectal, or intravenous).

The pharmaceutically acceptable carrier to be used in the production of the combination agent of the present invention may be any of those used for the pharmaceutical composition of the present invention.

A bleeding ratio the compound of the present invention to the combination drug in the combination agent of the present invention can be appropriately determined depending on the target of administration, the route of administration, diseases, and the like. Two or more of the combination drugs as described above may be combined together at an appropriate ratio. The dosage of the combination agent can be appropriately determined on the basis of a clinically used dosage. For example, if the target of administration is a human, 0.01 to 100 parts by weight of the combination drug may be used for one part by weight of the compound of the present invention.

For instance, the content of the compound of the present invention in the combination agent of the present invention varies among the dosage forms. In general, however, the content of the compound of the present invention is in the range of about 0.01 to 99.9% by weight, preferably about 0.01 to 99.9% by weight, more preferably about 0.5 to 20% by weight with respect to the whole amount of the pharmaceutical agent.

The content of the combination drug of the present invention in the combination agent of the present invention varies among the dosage forms. In general, however, the content of the compound of the present invention is in the range of about 0.01 to 99.9% by weight, preferably about 0.01 to 99.9% by weight, more preferably about 0.5 to 20% by weight with respect to the whole amount of the pharmaceutical agent.

The content of any additive such as a carrier in the combination agent of the present invention varies among the dosage forms. In general, however, the content of the additive is in the range of about 1 to 99.99% by weight, preferably about 10 to 90% by weight with respect to the whole amount of the pharmaceutical agent.

The contents of the compound of the present invention and the combination drug may be equal to those described above even if they are independently formulated.

As described above, the dosage varies under various conditions, so that the contents of the compound of the present invention and the combination drug may be less than the above dosages or may be higher than the above dosages in some cases.

### Examples

The present invention will be illustrated in further detail by the following reference examples, working examples, preparation example, and test example, but these examples, which are merely embodiments, do not limit the present invention and may be modified without departing from the scope of the invention.

In the following reference examples and working examples, “room temperature” ordinarily indicates a temperature from about 10° C. to about 35° C. Percentages for yield indicate mol/mol % and percentages for media used in chromatography indicate percent by volume, but otherwise indicate percent by weight. Broad peaks such as OH and NH protons that could not be confirmed in the proton NMR spectra are not included in the data. Kiesselgel 60 by Merck was used in silica gel chromatography, and Chromatorex NH by Fuji Silysia Chemical Ltd. was used in basic silica gel chromatography.

Other abbreviations used in this document are defined below.

s: singlet

d: doublet

dd: doublet of doublets

dt: doublet of triplets

t: triplet

tt: triplet of triplets

td: triplet of doublets

q: quartet

septet

m: multiplet

br: broad

J: coupling constant

Hz: Hertz

CDCl₃: deuterated chloroform

DMSO-d₆: deuterated dimethyl sulfoxide

¹H-NMR: proton nuclear magnetic resonance

HPLC: high performance liquid chromatography

THF: tetrahydrofuran

DMF: N,N-dimethyl formamide

DMSO: dimethyl sulfoxide

NMP: N-methyl pyrrolidone

HOBt: 1-hydroxybenzotriazole

WSC: 1-ethyl-3-(3-dimethylaminopropyl)carbodiimide hydrochloride

HATU: 2-(7-aza-1H-benzotriazol-1-yl)-1,1,3,3-tetramethyluronium hexafluorophosphate

DMTMM: 4-(4,6-dimethoxy-1,3,5-triazin-2-yl)-4-methylmorpholinium chloride n-hydrate

DBU: 1,8-diazabicyclo[5.4.0]-7-undecene

LC-MS: liquid chromatography-mass spectrometry

ESI: electrospray ionization

### Reference Example 1

### 8-Chloro-2-[3-(trifluoromethyl)benzyl]-1,2,3,4-tetrahydroisoquinoline

A mixture of 8-chloro-1,2,3,4-tetrahydroisoquinoline (4.5 g, 22.0 mmol), 3-trifluoromethylbenzaldehyde (3.25 mL, 24.3 mmol), sodium acetate (1.81 g, 22.0 mmol), and acetic acid (0.63 mL, 11.0 mmol) in ethanol was stirred for 1 hour. Sodium triacetoxyborohydride (5.60 g, 26.5 mmol) was added a little at a time over a period of 30 min, and the mixture was then stirred for 15 hours. The reaction solution was diluted with saturated aqueous sodium bicarbonate and extracted with ethyl acetate. The extract was washed with water and dried over anhydrous magnesium sulfate, and the solvent was then distilled off at reduced pressure. The residue was purified by silica gel column chromatography (hexane-ethyl acetate=10:1), the resulting oily substance was dissolved in isopropyl alcohol (30 mL), and 4 N hydrogen chloride/ethyl acetate (5 mL) was added. The precipitated crystals were filtered off to give 5.89 g of the titled compound (yield 74%). Melting point: 217-218° C.

¹H-NMR (DMSO-d₆) δ: 3.00-3.40 (4H, m), 4.20-4.40 (2H, m), 4.50-4.80 (2H, m), 7.25 (1H, d, J=7.5 Hz), 7.32 (1H, d, J=7.2 Hz), 7.38 (1H, t, J=7.2 Hz), 7.74 (1H, t, J=7.5 Hz), 7.80-7.90 (1H, m), 7.90-8.00 (1H, m), 8.10 (1H, s), 11.24 (1H, br s).

### Reference Example 2

### Ethyl 3-[2-[3-(trifluoromethyl)benzyl]-1,2,3,4-tetrahydroisoquinolin-8yl]benzoate

A mixture of 8-chloro-2-[3-(trifluoromethyl)benzyl]-1,2,3,4-tetrahydroisoquinoline (0.36 g, 1.0 mmol) obtained in Reference Example 1, [3-(ethoxycarbonyl)phenyl]boronic acid (0.23 g, 1.2 mmol), Neolyst CX-32 (cinnamylchloro[1,3-bis-(diisopropylphenyl)-2-imidazolidinylidene]palladium (II)) (19.5 mg, 0.03 mmol), and potassium tert-butoxide (0.26 g, 2.3 mmol) in dimethoxyethane (5 mL) was stirred for 13 hours at 90° C. The reaction solution was diluted with saturated aqueous sodium bicarbonate and extracted with ethyl acetate. The extract was washed with water and dried over anhydrous magnesium sulfate, and the solvent was then distilled off at reduced pressure. The residue was purified by silica gel column chromatography (hexane-ethyl acetate=10:1) to give 0.24 g of the titled compound (yield 55%) in the form of an oily substance.

¹H-NMR (CDCl₃) δ: 1.39 (3H, t, J=7.2 Hz), 2.68 (2H, t, J=6.0 Hz), 2.96 (2H, t, J=6.0 Hz), 3.51 (1H, s), 3.61 (3H, s), 4.38 (2H, q, J=7.2 Hz), 7.03 (1H, d, J=7.2 Hz), 7.14 (1H, d, J=7.8 Hz), 7.20 (1H, d, J=7.5 Hz), 7.30-7.50 (5H, m), 7.54 (1H, s), 7.95 (1H, s), 7.97-8.05 (1H, m).

### Reference Example 3

### 5-Bromo-2-[3-(trifluoromethyl)benzyl]-1,2,3,4-tetrahydroisoquinoline

Sodium triacetoxyborohydride (5.81 g, 27.4 mmol) was added to a THF (100 mL)-DMF (10 mL) mixed solution of 5-bromo-1,2,3,4-tetrahydroisoquinoline (2.90 g, 13.7 mmol) and 3-(trifluoromethyl)benzaldehyde (2.74 mL, 20.6 mmol), and the mixture was stirred for 15 hours at room temperature. The reaction solution was treated with the addition of water and 1 N sodium hydroxide aqueous solution, and was extracted with ethyl acetate. The organic layer was washed with saturated saline, dried over anhydrous sodium sulfate, and then concentrated at reduced pressure. The residue was purified by silica gel column chromatography to give 4.77 g of the titled compound (yield 94%) in the form of an oily substance.

¹H-NMR (CDCl₃) δ: 2.72-2.80 (2H, m), 2.82-2.92 (2H, m), 3.62 (2H, s), 3.72 (2H, s), 6.91-6.96 (1H, m), 6.96-7.03 (1H, m), 7.40 (1H, dd, J=7.5, 1.3 Hz), 7.46 (1H, d, J=7.5 Hz), 7.51-7.56 (1H, m), 7.58 (1H, d, J=7.5 Hz), 7.65 (1H, s)

### Reference Example 4

### Ethyl 3-[2-[3-(trifluoromethyl)benzyl]-1,2,3,4-tetrahydroisoquinolin-5-yl]benzoate

A mixture of the 5-bromo-2-[3-(trifluoromethyl)benzyl]-1,2,3,4-tetrahydroisoquinoline (4.76 g, 12.9 mmol) obtained in Reference Example 3, [3-(ethoxycarbonyl)phenyl]boronic acid (3.01 g, 15.5 mmol), and tetrakis(triphenylphosphine)palladium (0) (601 mg, 0.52 mmol) in 2 N sodium carbonate aqueous solution (25.8 mL)-1,2-dimethoxyethane (50 mL) was reacted for 20 hours at 90° C. in a nitrogen atmosphere. The addition of saturated saline to the reaction solution was followed by extraction with ethyl acetate. The organic layer was washed with water, dried over anhydrous sodium sulfate, and then concentrated at reduced pressure. The residue was purified by silica gel column chromatography to give 5.20 g of the titled compound (yield 92%) in the form of an oily substance.

¹H-NMR (CDCl₃) δ: 1.39 (3H, t, J=7.2 Hz), 2.62-2.77 (4H, m), 3.72 (4H, s), 4.38 (2H, q, J=7.0 Hz), 7.04 (1H, d, J=7.5 Hz), 7.06-7.12 (1H, m), 7.16-7.24 (1H, m), 7.41-7.56 (4H, m), 7.60 (1H, d, J=7.5 Hz), 7.67 (1H, s), 7.98-8.05 (2H, m).

### Reference Example 5

### 3-[2-[3-(Trifluoromethyl)benzyl]-1,2,3,4-tetrahydroisoquinolin-5-yl]benzoic acid

2 N sodium hydroxide aqueous solution (11.4 m, 22.8 mmol) was added to a THF (40 mL)-methanol (20 mL) mixed solution of ethyl 3-[2-[3-(trifluoromethyl)benzyl]-1,2,3,4-tetrahydroisoquinolin-5-yl]benzoate (5.00 g, 11.4 mmol) obtained in Reference Example 4, and the mixture was stirred for 6 hours at room temperature. The reaction solution was neutralized with the addition of 6 N hydrochloric acid, diluted with water, and then extracted with ethyl acetate. The organic layer was washed with water, dried over anhydrous magnesium sulfate, and then concentrated at reduced pressure. The precipitated crystals were filtered off with the addition of ethyl acetate to give 2.80 g of the titled compound (yield 60%) in the form of crystals. Melting point: 230-231° C.

¹H-NMR (DMSO-d₆) δ: 2.77 (1H, d, J=19.3 Hz), 3.14 (1H, d, J=22.3 Hz), 3.28 (1H, br s), 3.58 (1H, br s), 4.40 (2H, br s), 4.60 (2H, d, J=3.4 Hz), 7.21-7.32 (2H, m), 7.33-7.43 (1H, m), 7.54-7.66 (2H, m), 7.74 (1H, t, J=7.6 Hz), 7.82-7.89 (2H, m), 7.98 (2H, d, J=7.2 Hz), 8.10 (1H, br s), 11.18 (1H, br s).

### Reference Example 6

### 8-Chloro-4-methyl-2-[3-(trifluoromethyl)benzyl]-1,2,3,4-tetrahydroisoquinoline

8-Chloro-4-methyl-2-[3-(trifluoromethyl)benzyl]-1,2,3,4-tetrahydroisoquinoline was used in the same manner as in Reference Example 1 to obtain the titled compound. 73% yield, oily substance.

¹H-NMR (CDCl₃) δ: 1.29 (3H, d, J=6.9 Hz), 2.43 (1H, dd, J=11.4, 5.4 Hz), 2.66 (1H, dd, J=11.4, 4.8 Hz), 2.90-3.05 (1H, m), 3.60 (1H, d, J=15.6 Hz), 3.70 (1H, d, J=13.8 Hz), 3.78 (1H, d, J=13.2 Hz), 3.82 (1H, d, J=13.2 Hz), 7.10-7.20 (3H, m), 7.44 (1H, t, J=7.7 Hz), 7.53 (1H, d, J=7.7 Hz), 7.58 (1H, d, J=7.2 Hz), 7.67 (1H, s).

### Reference Example 7

### Ethyl 3-[4-methyl-2-[3-(trifluoromethyl)benzyl]-1,2,3,4-tetrahydroisoquinolin-8yl]benzoate

8-Chloro-4-methyl-2-[3-(trifluoromethyl)benzyl]-1,2,3,4-tetrahydroisoquinoline obtained in Reference Example 6 was used in the same manner as in Reference Example 2 to obtain the titled compound. 60% yield, oily substance.

¹H-NMR (CDCl₃) δ: 1.33 (3H, t, J=7.2 Hz), 1.39 (3H, t, J=7.1 Hz), 2.38 (1H, dd, J=11.4, 6.0 Hz), 2.68 (1H, dd, J=11.4, 5.1 Hz), 3.00-3.10 (1H, m), 3.45 (1H, d, J=15.3 Hz), 3.54 (2H, d, J=13.8 Hz), 3.63 (1H, d, J=13.8 Hz), 4.38 (2H, q, J=7.2 Hz), 7.03 (1H, t, J=4.5 Hz), 7.27 (1H, s), 7.30-7.55 (6H, m), 7.57 (1H, s), 7.97 (1H, s), 8.00-8.10 (1H, m).

### Reference Example 8

### (4-Bromo-1-benzofuran-2-yl)[3-(trifluoromethyl)phenyl]methanone

2-Bromo-1-[3-(trifluoromethyl)phenyl]ethanone (6.38 g, 23.9 mmol) and potassium carbonate (3.30 g, 23.9 mmol) were added at room temperature to an acetonitrile (50 mL) solution of 2-bromo-6-hydroxybenzaldehyde (4.0 g, 19.9 mmol), and the mixture was heated to reflux for 16 hours. The reaction solution was added to water and extracted with ethyl acetate. The organic layer was washed with water and saturated saline, then dried over anhydrous sodium sulfate, and concentrated at reduced pressure. The residue was purified by silica gel column chromatography (hexane-ethyl acetate 90:10→40:60) to give 4.4 g of the titled compound (yield 60%). Melting point: 91-92° C. (methanol).

¹H-NMR (CDCl₃) δ: 7.39 (1H, t, J=8.1 Hz), 7.49-7.62 (3H, m), 7.71 (1H, t, J=7.5 Hz), 7.91 (1H, d, J=7.8, 1.2 Hz), 8.25 (1H, d, J=7.8 Hz), 8.32 (1H, s).

### Reference Example 9

### 4-Bromo-2-[3-(trifluoromethyl)benzyl]-1-benzofuran

A mixture of (4-bromo-1-benzofuran-2-yl)[3-(trifluoromethyl)phenyl]methanone (4.4 g, 11.9 mmol) obtained in Reference Example 8 and hydrazine monohydrate (2.38 g, 47.6 mmol) in ethylene glycol (50 mL) was heated to 130° C. for 2 hours. The mixture was allowed to cool to room temperature, potassium hydroxide (2.00 g, 35.7 mmol) was then added, and the mixture was heated to 160° C. for 2 hours. The reaction solution was added to water and extracted with ethyl acetate. The organic layer was washed with water and saturated saline, then dried over anhydrous sodium sulfate, and concentrated at reduced pressure. The residue was purified by silica gel column chromatography (hexane-ethyl acetate 95:5→30:70) to give 3.4 g of the titled compound (yield 80%) in the form of an oily substance.

¹H-NMR (CDCl₃) δ: 4.16 (2H, s), 6.46 (1H, s), 7.09 (1H, t, J=8.1 Hz), 7.31-7.36 (2H, m), 7.40-7.61 (4H, m).

### Reference Example 10

### 3-[2-[3-(Trifluoromethyl)benzyl]-1-benzofuran-4-yl]benzoic acid

A mixture of 4-bromo-2-[3-(trifluoromethyl)benzyl]-1-benzofuran (3.40 g, 9.58 mmol) obtained in Reference Example 4, [3-(ethoxycarbonyl)phenyl]boronic acid (2.23 g, 11.5 mmol), and tetrakis(triphenylphosphine)palladium (0) (553 mg, 0.48 mmol) in 2 N sodium carbonate aqueous solution (30 mL)-1,2-dimethoxyethane (30 mL) was reacted for 16 hours at 90° C. in a nitrogen atmosphere. The addition of water to the reaction solution was followed by extraction with ethyl acetate. The organic layer was washed with saturated saline, then dried over anhydrous sodium sulfate, and then concentrated at reduced pressure, and the residue was purified by silica gel column chromatography (ethyl acetate-hexane 2:3) to give 3.30 g of 3-[2-[3-(trifluoromethyl)benzyl]-1-benzofuran-4-yl]benzoic acid. 2 N sodium hydroxide aqueous solution (8 mL, 16 mmol) was added at room temperature to an ethanol (50 mL) solution of this compound, the mixture was stirred for 2 hours at 60° C., and the product was concentrated at reduced pressure. The reaction solution was made acidic with the addition of water and hydrochloric acid, and was extracted with ethyl acetate. The organic layer was washed with water and saturated saline, and then dried over anhydrous sodium sulfate. The solvent was distilled off at reduced pressure, and the resulting residue was crystallized from ethyl acetate-hexane to give 2.3 g of the titled compound (yield 61%). Melting point: 138-139° C. (ethyl acetate-hexane).

¹H-NMR (CDCl₃) δ: 4.19 (2H, s), 6.63 (1H, s), 7.20-7.78 (8H, m), 7.85 (1H, dd, J=7.8, 1.2 Hz), 8.13 (1H, dd, J=7.8, 1.2 Hz), 8.37 (1H, s), 1H unconfirmed.

### Reference Example 11

### (4-Methoxy-3-methyl-1-benzofuran-2-yl)[3-(trifluoromethyl)phenyl]methanone

1-(2-Hydroxy-6-methoxyphenyl)ethanone was used in the same manner as in Reference Example 8 to obtain the titled compound. Yield: 69%; melting point: 91-92° C. (methanol).

¹-NMR (CDCl₃) δ: 2.82 (3H, s), 3.96 (3H, s), 6.66 (1H, d, J=8.1 Hz), 7.09 (1H, d, J=8.4 Hz), 7.39 (1H, t, J=8.1 Hz), 7.64 (1H, t, J=8.1 Hz), 7.82 (1H, d, J=8.1 Hz), 8.24 (1H, dd, J=7.8 Hz), 8.31 (1H, s).

### Reference Example 12

### 4-Methoxy-3-methyl-2-[3-(trifluoromethyl)benzyl]-1-benzofuran

(4-Methoxy-3-methyl-1-benzofuran-2-yl)[3-(trifluoromethyl)phenyl]methanone obtained in Reference Example 11 was used in the same manner as in Reference Example 9 to obtain the titled compound. 74% yield, oily substance.

¹H-NMR (CDCl₃) δ: 2.37 (3H, s), 3.89 (3H, s), 4.08 (2H, s), 6.59 (1H, d, J=8.1 Hz), 6.97 (1H, d, J=7.8 Hz), 7.08 (1H, t, J=7.5 Hz), 7.35-7.50 (4H, m).

### Reference Example 13

### 3-Methyl-2-[3-(trifluoromethyl)benzyl]-1-benzofuran-4-ol

Boron tribromide (1.0 M methylene chloride solution, 5.0 mL, 5.0 mmol) was added dropwise at 0° C. in an argon atmosphere to a methylene chloride (30 mL) solution of 4-methoxy-3-methyl-2-[3-(trifluoromethyl)benzyl]-1-benzofuran (1.51 g, 4.68 mmol) obtained in Reference Example 12, and the mixture was stirred for 3 hours. The reaction solution was added to saturated sodium bicarbonate aqueous solution, the organic layer was separated, and the aqueous layer was extracted with ethyl acetate. All of the organic layer was washed with saturated saline, then dried over anhydrous sodium sulfate, filtered, and concentrated at reduced pressure. The residue was purified by silica gel column chromatography (hexane-ethyl acetate 90:10→20:80) to give 1.38 g of the titled compound (yield 96%). Melting point: 91-92° C. (ethyl acetate-hexane). ¹H-NMR (CDCl₃) δ: 2.41 (3H, s), 4.09 (2H, s), 5.05 (1H, s), 6.50 (1H, d, J=8.4 Hz), 6.92-7.05 (2H, m), 7.30-7.51 (4H, m).

### Reference Example 14

### 3-Methyl-2-[3-(trifluoromethyl)benzyl]-1-benzofuran-4-yl trifluoromethanesulfonate

Trifluoromethanesulfonic anhydride (0.83 mL, 4.95 mmol) was added at 0° C. to a pyridine (15 mL) solution of 3-methyl-2-[3-(trifluoromethyl)benzyl]-1-benzofuran-4-ol (1.38 g, 4.50 mmol) obtained in Reference Example 13, and the mixture was stirred for 4 hours at room temperature. Water was added to the reaction solution, the organic layer was separated, and the aqueous layer was extracted with ethyl acetate. The organic layer was washed with 1 N hydrochloric acid and saturated sodium bicarbonate aqueous solution, then dried over magnesium sulfate, filtered, and concentrated at reduced pressure. The residue was purified by silica gel column chromatography (hexane-ethyl acetate 90:10→50:50) to give 1.40 g of the titled compound (yield 71%) in the form of an oily substance.

¹H-NMR (CDCl₃) δ: 2.40 (3H, s), 4.13 (2H, s), 7.13 (1H, d, J=7.2 Hz), 7.23 (1H, t, J=8.1 Hz), 7.30-7.51 (5H, m).

### Reference Example 15

### 3-[3-Methyl-2-[3-(trifluoromethyl)benzyl]-1-benzofuran-4-yl]benzoic acid

3-Methyl-2-[3-(trifluoromethyl)benzyl]-1-benzofuran-4-yl trifluoromethanesulfonate obtained in Reference Example 14 was used in the same manner as in Reference Example 10 to obtain the titled compound. Yield: 72%; melting point: 169-170° C. (ethyl acetate-hexane).

¹H-NMR (CDCl₃) δ: 1.82 (3H, s), 4.12 (2H, s), 7.10 (1H, dd, J=7.2, 1.2 Hz), 7.23-7.30 (1H, m), 7.37-7.57 (6H, m), 7.67 (1H, dt, J=7.5, 1.5 Hz), 8.13 (1H, dd, J=7.8, 1.5 Hz), 8.17 (1H, t, J=1.5 Hz), 1H unconfirmed.

### Reference Example 16

### (4-Bromo-7-methoxy-1-benzofuran-2-yl)[3-(trifluoromethyl)phenyl]methanone

6-Bromo-2-hydroxy-3-methoxybenzaldehyde was used in the same manner as in Reference Example 8 to obtain the titled compound. Yield: 58%; melting point: 78-79° C. (methanol).

¹H-NMR (CDCl₃) δ: 4.03 (3H, s), 6.87 (1H, d, J=8.4 Hz), 7.39 (1H, d, J=8.1 Hz), 7.57 (1H, s), 7.70 (1H, t, J=7.8 Hz), 7.90 (1H, d, J=7.8 Hz), 8.29 (1H, d, J=7.8 Hz), 8.36 (1H, s).

### Reference Example 17

### 4-Bromo-7-methoxy-2-[3-(trifluoromethyl)benzyl]-1-benzofuran

(4-Bromo-7-methoxy-1-benzofuran-2-yl)[3-(trifluoromethyl)phenyl]methanone obtained in Reference Example 16 was used in the same manner as in Reference Example 9 to obtain the titled compound.

85% yield, oily substance.

¹H-NMR (CDCl₃) δ: 3.96 (3H, s), 4.18 (2H, s), 6.40 (1H, s), 6.64 (1H, d, J=8.4 Hz), 7.24 (1H, d, J=8.1 Hz), 7.41-7.58 (4H, m).

### Reference Example 18

### 3-[7-Methoxy-2-[3-(trifluoromethyl)benzyl]-1-benzofuran-4-yl]benzoic acid

4-Bromo-7-methoxy-2-[3-(trifluoromethyl)benzyl]-1-benzofuran obtained in Reference Example 17 was used in the same manner as in Reference Example 10 to obtain the titled compound. Yield: 44%; melting point: 118-119° C. (ethyl acetate-hexane).

¹H-NMR (CDCl₃) δ: 4.04 (3H, s), 4.21 (2H, s), 6.60 (1H, s), 6.86 (1H, d, J=8.1 Hz), 7.23-7.28 (1H, m), 7.38-7.60 (5H, m), 7.76-7.82 (1H, m), 8.08 (1H, d, J=7.8 Hz), 8.32 (1H, t, J=1.5 Hz), 1H unconfirmed.

### Reference Example 19

### 4-(4,4,5,5-tetramethyl-1,3,2-dioxaborolan-2-yl)-2-[3-(trifluoromethyl)benzyl]-1-benzofuran

(1,1-Bis(diphenylphosphino)ferrocene)dichloropalladium (II) complex with dichloromethane (114 mg, 0.14 mmol) was added to a mixture of 4-bromo-2-[3-(trifluoromethyl)benzyl]-1-benzofuran (1.00 g, 2.82 mmol) obtained in Reference Example 9, 4,4,4,4′,4′,5,5,5′,5′-octamethyl-2,2′-bi-1,3,2-dioxaborolane (858 mg, 3.38 mmol), and potassium acetate (332 mg, 3.38 mmol) in DMSO (15 mL), and the mixture was heated and stirred for 20 hours at 85° C. Ethyl acetate was poured into the reaction solution, and the mixture was washed with saturated saline, dried over anhydrous magnesium sulfate, and then concentrated at reduced pressure. The residue was purified by silica gel column chromatography (hexane-ethyl acetate 100:0→95:5) to give 677 mg of the titled compound (yield 60%) in the form of an oily substance.

¹H-NMR (CDCl₃) δ: 1.37 (12H, s), 4.18 (2H, s), 6.94 (1H, d, J=0.8 Hz), 7.19-7.25 (1H, m), 7.42-7.47 (2H, m), 7.49 (1H, d, J=0.9 Hz), 7.52 (1H, d, J=1.7 Hz), 7.59 (1H, s), 7.67 (1H, dd, J=7.2, 0.9 Hz).

### Reference Example 20

### Methyl 2-[2-[3-(trifluoromethyl)benzyl]-1-benzofuran-4-yl]isonicotinate

4-(4,4,5,5-tetramethyl-1,3,2-dioxaborolan-2-yl)-2-[3-(trifluoromethyl)benzyl]-1-benzofuran obtained in Reference Example 19 and methyl 2-bromoisonicotinate were used in the same manner as in Reference Example 4 to obtain the titled compound. 43% yield, oily substance.

¹H-NMR (CDCl₃) δ: 3.99 (3H, s), 4.21 (2H, s), 7.11 (1H, s), 7.35 (1H, t, J=8.0 Hz), 7.40-7.47 (1H, m), 7.51 (3H, t, J=7.0 Hz), 7.60 (1H, s), 7.69 (1H, d, J=7.6 Hz), 7.79 (1H, dd, J=5.1, 1.3 Hz), 8.32 (1H, s), 8.88 (1H, d, J=4.9 Hz).

### Reference Example 21

### 2-[2-[3-(Trifluoromethyl)benzyl]-1-benzofuran-4-yl]isonicotinic acid

Methyl 2-[2-[3-(trifluoromethyl)benzyl]-1-benzofuran-4-yl]isonicotinate obtained in Reference Example 20 was used in the same manner as in Reference Example 5 to obtain the titled compound. Yield: 68%; melting point: 187-188° C. (ethyl acetate).

¹H-NMR (DMSO-d₆) δ: 4.36 (2H, s), 7.25 (1H, s), 7.39 (1H, t, J=8.0 Hz), 7.54-7.69 (4H, m), 7.74 (1H, s), 7.78-7.82 (2H, m), 8.28 (1H, s), 8.91 (1H, d, J=5.7 Hz), 13.79 (1H, s).

### Reference Example 22

### (4-Chlorofuro[3,2-c]pyridin-2-yl)[3-(trifluoromethyl)phenyl]methanol

1.58 N t-butyllithium (791 μL, 1.25 mmol) was added at −78° C. to a THF solution (5.0 mL) of 4-chlorofuro[3,2-c]pyridine (153 mg, 1.00 mmol), and the mixture was stirred for 10 min. A THF solution (5.0 mL) of 3-(trifluoromethyl)benzaldehyde was stirred into the reaction solution for 15 min at −78° C., and the mixture was then stirred for 45 min at room temperature. The addition of saturated sodium bicarbonate aqueous solution to the reaction solution was followed by extraction with ethyl acetate. The organic layer was washed with saturated saline, and then dried over anhydrous sodium sulfate. The solvent was distilled off at reduced pressure, and the resulting residue was purified by silica gel column chromatography (hexane-ethyl acetate 65:35→50:50) to give 311 mg of the titled compound (yield 95%).

¹H-NMR (CDCl₃) δ: 2.77 (1H, d, J=4.4 Hz), 6.04 (1H, d, J=4.4 Hz), 6.70 (1H, t, 0.8 Hz), 7.33 (1H, dd, J=5.8, 0.8 Hz), 7.56 (1H, d, J=7.7 Hz), 7.62-7.71 (2H, m), 7.79 (1H, s), 8.24 (1H, d, J=5.8 Hz).

### Reference Example 23

### 4-Chloro-2-[3-(trifluoromethyl)benzyl]furo[3,2-c]pyridine

Triethylsilane (453 μL, 2.84 mmol) was added to a trifluoroacetic acid solution (3.0 mL) of (4-chlorofuro[3,2-c]pyridin-2-yl)[3-(trifluoromethyl)phenyl]methanol (310 mg, 0.946 mmol) obtained in Reference Example 22, the mixture was stirred for 14 hours at room temperature, triethylsilane (453 μL, 2.84 mmol) was then added, and the mixture was stirred for 4 hours at 80° C. The reaction solution was made basic using saturated sodium bicarbonate aqueous solution, and was extracted with ethyl acetate. The organic layer was washed with saturated saline and was then dried over anhydrous sodium sulfate. The solvent was distilled off at reduced pressure, and the resulting residue was purified by silica gel column chromatography (hexane-ethyl acetate 9:1→2:1) to give a crude preparation of the titled compound (269 mg). A portion (169 mg) of the resulting crude preparation (269 mg) was purified by silica gel column chromatography (hexane-ethyl acetate 95:5→70:30) to give 121 mg of the titled compound (yield 65%). ¹H-NMR (CDCl₃) δ: 4.19 (2H, s), 6.50-6.53 (1H, m), 7.29-7.32 (1H, m), 7.46-7.51 (2H, m), 7.53-7.59 (2H, m), 8.21 (1H, d, J=5.8 Hz).

### Reference Example 24

### Ethyl 3-[2-[3-(trifluoromethyl)benzyl]furo[3,2-c]pyridin-4-yl]benzoate

4-Chloro-2-[3-(trifluoromethyl)benzyl]furo[3,2-c]pyridine obtained in Reference Example 23 and [3-(ethoxycarbonyl)phenyl]boronic acid were used in the same manner as in Reference Example 4 to obtain the titled compound. Yield: 87%.

¹H-NMR (CDCl₃) δ: 1.41 (3H, t, J=7.1 Hz), 4.22 (2H, s), 4.41 (2H, q, J=7.1 Hz), 6.76 (1H, d, J=0.8 Hz), 7.34-7.37 (1H, m), 7.43-7.63 (5H, m), 8.09-8.16 (2H, m), 8.54 (1H, d, J=5.5 Hz), 8.58 (1H, t, J=1.6 Hz).

### Reference Example 25

### 3-[2-[3-(Trifluoromethyl)benzyl]furo[3,2-c]pyridin-4-yl]benzoic acid

Ethyl 3-[2-[3-(trifluoromethyl)benzyl]furo[3,2-c]pyridin-4-yl]benzoate obtained in Reference Example 24 was used in the same manner as in Reference Example 5 to obtain the titled compound. Yield: quantitative.

¹H-NMR (CDCl₃) δ: 4.26 (2H, s), 6.83 (1H, s), 7.44-7.60 (5H, m), 7.66 (1H, t, J=7.7 Hz), 8.20 (2H, dd, J=8.0, 1.6 Hz), 8.71 (1H, s), 8.75 (1H, d, J=5.8 Hz), 1H unconfirmed.

### Reference Example 26

### (7-Bromo-1-benzofuran-2-yl)[3-(trifluoromethyl)phenyl]methanone

3-Bromo-2-hydroxybenzaldehyde was used in the same manner as in Reference Example 8 to obtain the titled compound. Yield: 69%; melting point: 75-78° C. (methanol).

¹H-NMR (CDCl₃) δ: 7.24 (1H, t, J=7.5 Hz), 7.66-7.74 (4H, m), 7.90 (1H, d, J=7.8 Hz), 8.36 (1H, d, J=7.8 Hz), 8.50 (1H, s).

### Reference Example 27

### 7-Bromo-2-[3-(trifluoromethyl)benzyl]-1-benzofuran

(7-Bromo-1-benzofuran-2-yl)[3-(trifluoromethyl)phenyl]methanone obtained in Reference Example 26 was used in the same manner as in Reference Example 9 to obtain the titled compound. 74% yield, oily substance.

¹H-NMR (CDCl₃) δ: 4.20 (2H, s), 6.40 (1H, s), 7.06 (1H, t, J=7.5 Hz), 7.35-7.59 (6H, m).

### Reference Example 28

### 3-[2-[3-(Trifluoromethyl)benzyl]-1-benzofuran-7-yl]benzoic acid

7-Bromo-2-[3-(trifluoromethyl)benzyl]-1-benzofuran obtained in Reference Example 27 was used in the same manner as in Reference Example 10 to obtain the titled compound. Yield: 72%; melting point: 169-170° C. (ethyl acetate-hexane).

¹H-NMR (CDCl₃) δ: 4.19 (2H, s), 6.47 (1H, s), 7.29 (1H, t, J=7.5 Hz), 7.38-7.62 (7H, m), 8.07 (1H, d, J=7.8 Hz), 8.14 (1H, d, J=7.8 Hz), 8.60 (1H, t, J=1.5 Hz), 1H unconfirmed.

### Reference Example 29

### 1-(3-Bromo-2-hydroxyphenyl)ethanone

N-Bromosuccinimide (5.25 g, 29.5 mmol) was added at 0° C. to a mixture of 1-(2-hydroxyphenyl)ethanone (4.00 g, 29.5 mmol) and diisopropylamine (0.42 mL, 2.95 mmol) in carbon disulfide (50 mL), and the mixture was stirred for 1 hour at room temperature. Water was poured into the reaction mixture, and the mixture was extracted with ethyl acetate. The extract was washed with saturated sodium bicarbonate aqueous solution and water, then dried over magnesium sulfate, filtered, and concentrated at reduced pressure, and the residue was purified by silica gel column chromatography (ethyl acetate-hexane 0:10→2:8) to give 1.60 g of the titled compound (yield 25%).

¹H-NMR (CDCl₃) δ: 2.66 (3H, s), 6.82 (1H, t, J=7.8 Hz), 7.69-7.75 (2H, m), 12.9 (1H, s).

### Reference Example 30

### (7-Bromo-3-methyl-1-benzofuran-2-yl)[3-(trifluoromethyl)phenyl]methanone

1-(3-Bromo-2-hydroxyphenyl)ethanone obtained in Reference Example 29 was used in the same manner as in Reference Example 8 to obtain the titled compound. Yield: 88%; melting point: 97-98° C. (methanol).

¹H-NMR (CDCl₃) δ: 2.70 (3H, s), 7.20-7.27 (1H, m), 7.63-7.71 (3H, m), 7.87 (1H, d, J=7.8 Hz), 8.39 (1H, d, J=7.8 Hz), 8.57 (1H, s).

### Reference Example 31

### 7-Bromo-3-methyl-2-[3-(trifluoromethyl)benzyl]-1-benzofuran

(7-Bromo-3-methyl-1-benzofuran-2-yl)[3-(trifluoromethyl)phenyl]methanone obtained in Reference Example 30 was used in the same manner as in Reference Example 9 to obtain the titled compound. 75% yield, oily substance.

¹H-NMR (CDCl₃) δ: 2.20 (3H, s), 4.18 (2H, s), 7.08 (1H, t, J=7.5 Hz), 7.34-7.53 (6H, m).

### Reference Example 32

### 3-[3-Methyl-2-[3-(trifluoromethyl)benzyl]-1-benzofuran-7-yl]benzoic acid

7-Bromo-3-methyl-2-[3-(trifluoromethyl)benzyl]-1-benzofuran obtained in Reference Example 31 was used in the same manner as in Reference Example 10 to obtain the titled compound. Yield: 70%; melting point: 200-201° C. (ethyl acetate-hexane).

¹H-NMR (CDCl₃) δ: 2.28 (3H, s), 4.17 (2H, s), 7.32 (1H, t, J=7.8 Hz), 7.40-7.60 (7H, m), 8.05 (1H, dt, J=7.5, 1.5 Hz), 8.11 (1H, dt, J=7.8, 1.5 Hz), 8.58 (1H, t, J=1.5 Hz), 1H unconfirmed.

### Reference Example 33

### Methyl 2-[3-methyl-2-[3-(trifluoromethyl)benzyl]-1-benzofuran-7-yl]pyridine-4-carboxylate

7-Bromo-3-methyl-2-[3-(trifluoromethyl)benzyl]-1-benzofuran obtained in Reference Example 31 was used in the same manner as in Reference Example 19 to obtain 3-methyl-7-(4,4,5,5-tetramethyl-1,3,2-dioxaborolan-2-yl)-2-[3-(trifluoromethyl)benzyl]-1-benzofuran (890 mg) in the form of an oily substance, and 3-methyl-7-(4,4,5,5-tetramethyl-1,3,2-dioxaborolan-2-yl)-2-[3-(trifluoromethyl)benzyl]-1-benzofuran and methyl 2-bromopyridine-4-carboxylate were used in the same manner as in Reference Example 4 to obtain the titled compound in solid form. Yield: 45%.

¹H-NMR (CDCl₃) δ: 2.30 (3H, s), 3.97 (3H, s), 4.23 (2H, s), 7.33-7.41 (1H, m), 7.42-7.62 (5H, m), 7.78 (1H, dd, J=4.9, 1.5 Hz), 8.14 (1H, dd, J=7.6, 1.5 Hz), 8.81-8.84 (1H, m), 8.85-8.89 (1H, m).

### Reference Example 34

### 2-[3-Methyl-2-[3-(trifluoromethyl)benzyl]-1-benzofuran-7-yl]pyridine-4-carboxylic acid

Methyl 2-[3-methyl-2-[3-(trifluoromethyl)benzyl]-1-benzofuran-7-yl]pyridine-4-carboxylate obtained in Reference Example 33 was used in the same manner as in Reference Example 5 to obtain the titled compound in solid form. Yield: 85%.

¹H-NMR (DMSO-d₆) δ: 2.32 (3H, s), 4.35 (2H, s), 7.39 (1H, t, J=7.7 Hz), 7.57-7.68 (3H, m), 7.70-7.84 (3H, m), 8.13 (1H, dd, J=7.7, 1.2 Hz), 8.76-8.79 (1H, m), 8.90 (1H, dd, J=5.0, 1.2 Hz), 13.78 (1H, br s).

### Reference Example 35

### (7-Bromo-4-fluoro-1-benzofuran-2-yl)[3-(trifluoromethyl)phenyl]methanone

3-Bromo-6-fluoro-2-hydroxybenzaldehyde was used in the same manner as in Reference Example 8 to obtain the titled compound. Yield: 74%; melting point: 114-115° C. (methanol).

¹H-NMR (CDCl₃) δ: 6.97 (1H, t, J=8.7 Hz), 7.62 (1H, dd, J=8.7, 4.5 Hz), 7.71 (1H, t, J=7.8 Hz), 7.76 (1H, s), 7.91 (1H, d, J=7.8 Hz), 8.35 (1H, d, J=7.8 Hz), 8.49 (1H, s).

### Reference Example 36

### 7-Bromo-4-fluoro-2-[3-(trifluoromethyl)benzyl]-1-benzofuran

(7-Bromo-4-fluoro-1-benzofuran-2-yl)[3-(trifluoromethyl)phenyl]methanone obtained in Reference Example 35 was used in the same manner as in Reference Example 9 to obtain the titled compound. 79% yield, oily substance.

¹H-NMR (CDCl₃) δ: 4.21 (2H, s), 6.50 (1H, s), 6.81 (1H, t, J=8.7 Hz), 7.31 (1H, dd, J=8.7, 4.8 Hz), 7.45-7.64 (4H, m).

### Reference Example 37

### 3-[4-Fluoro-2-[3-(trifluoromethyl)benzyl]-1-benzofuran-7-yl]benzoic acid

7-Bromo-4-fluoro-2-[3-(trifluoromethyl)benzyl]-1-benzofuran obtained in Reference Example 36 was used in the same manner as in Reference Example 10 to obtain the titled compound. Yield: 70%; melting point: 172-173° C. (ethyl acetate-hexane).

¹H-NMR (CDCl₃) δ: 4.19 (2H, s), 6.56 (1H, s), 7.00 (1H, t, J=8.7 Hz), 7.38 (1H, dd, J=8.1, 4.8 Hz), 7.42-7.65 (5H, m), 7.97-8.02 (1H, m), 8.10-8.15 (1H, m), 8.53 (1H, t, J=1.5 Hz), 1H unconfirmed.

### Reference Example 38

### 3-[2-[3-(Trifluoromethyl)benzyl]-1-benzofuran-7-yl]aniline

A mixture of the 7-bromo-2-[3-(trifluoromethyl)benzyl]-1-benzofuran (2.00 g, 5.63 mmol) obtained in Reference Example 27, (3-aminophenyl)boronic acid (848 mg, 6.19 mmol), and tetrakis(triphenylphosphine)palladium (0) (325 mg, 0.28 mmol) in 2 N sodium carbonate aqueous solution (20 mL)-1,2-dimethoxyethane (20 mL) was reacted for 16 hours at 90° C. in a nitrogen atmosphere. The addition of water to the reaction solution was followed by extraction with ethyl acetate. The organic layer was washed with saturated saline, then dried over anhydrous sodium sulfate, and then concentrated at reduced pressure to give 1.80 g of the titled compound (yield 87%).

¹H-NMR (CDCl₃) δ: 3.72 (2H, br s), 4.18 (2H, s), 6.42 (1H, s), 6.67-6.74 (1H, m), 7.08-7.55 (9H, m), 7.63 (1H, s).

### Reference Example 39

### 3-[2-[3-(Trifluoromethyl)benzyl]-2,3-dihydro-1-benzofuran-7-yl]benzoic acid

Triethylsilane (0.8 mL, 5.0 mmol) was added at room temperature to a trifluoroacetic acid (10 mL) mixture of 3-[2-[3-(trifluoromethyl)benzyl]-1-benzofuran-7-yl]benzoic acid (1.00 g, 2.52 mmol) obtained in Reference Example 28, and the mixture was heated to reflux for 3 hours. The reaction solution was concentrated at reduced pressure, and the subsequent addition of saturated sodium bicarbonate aqueous solution to the residue was followed by extraction with ethyl acetate. The organic layer was washed with water and saturated saline, then dried over anhydrous sodium sulfate, and concentrated at reduced pressure. The resulting residue was crystallized from hexane to give 0.8 g of the titled compound (yield 80%). Melting point: 156-157° C. (hexane).

¹H-NMR (CDCl₃) δ: 2.99-3.11 (2H, m), 3.20-3.40 (2H, m), 4.90 (1H, br s), 5.00-5.12 (1H, m), 6.95 (1H, t, J=7.5 Hz), 7.16 (1H, d, J=7.2 Hz), 7.34 (1H, d, J=8.1 Hz), 7.38-7.54 (5H, m), 7.95 (1H, d, J7.8 Hz), 8.05 (1H, d, J=7.8 Hz), 8.46 (1H, s).

### Reference Example 40

### (7-Bromo-1-benzofuran-2-yl)(3-(fluorophenyl)methanone

3-Bromo-2-hydroxybenzaldehyde and 2-bromo-1-(3-fluorophenyl)ethanone were used in the same manner as in Reference Example 8 to obtain the titled compound. Yield: 76%.

¹H-NMR (CDCl₃) δ: 7.22 (1H, d, J=7.5 Hz), 7.35 (1H, dt, J=8.1, 2.7 Hz), 7.49-7.58 (1H, m), 7.64-7.71 (3H, m), 7.85 (1H, dd, J=7.8, 1.5 Hz), 7.97 (1H, d, J=7.8 Hz).

### Reference Example 41

### 7-Bromo-2-(3-fluorobenzyl)-1-benzofuran

(7-Bromo-1-benzofuran-2-yl)(3-(fluorophenyl)methanone obtained in Reference Example 40 was used in the same manner as in Reference Example 9 to obtain the titled compound. 62% yield, oily substance.

¹H-NMR (CDCl₃) δ: 4.14 (2H, s), 6.40 (1H, s), 6.90-7.18 (4H, m), 7.23-7.51 (3H, m).

### Reference Example 42

### 3-[2-(3-Fluorobenzyl)-1-benzofuran-7-yl]benzoic acid

7-Bromo-2-(3-fluorobenzyl)-1-benzofuran obtained in Reference Example 41 was used in the same manner as in Reference Example 10 to obtain the titled compound. Yield: 41%; melting point: 135-136° C. (ethyl acetate-hexane).

¹H-NMR (CDCl₃) δ: 4.14 (2H, s), 6.47 (1H, s), 6.92-7.17 (3H, m), 7.24-7.34 (2H, m), 7.42-7.51 (2H, m), 7.60 (1H, t, J=7.8 Hz), 8.06-8.17 (2H, m), 8.63 (1H, s), 1H unconfirmed.

### Reference Example 43

### (7-Bromo-1-benzofuran-2-yl)(3-methoxyphenyl)methanone

3-Bromo-2-hydroxybenzaldehyde and 2-bromo-1-(3-methoxyphenyl)ethanone were used in the same manner as in Reference Example 8 to obtain the titled compound. Yield: 91%.

¹H-NMR (CDCl₃) δ: 3.91 (3H, s), 7.14-7.26 (2H, m), 7.45 (1H, t, J=7.8 Hz), 7.62-7.79 (5H, m).

### Reference Example 44

### 7-Bromo-2-(3-methoxybenzyl)-1-benzofuran

(7-Bromo-1-benzofuran-2-yl)(3-methoxyphenyl)methanone obtained in Reference Example 43 was used in the same manner as in Reference Example 9 to obtain the titled compound. 68% yield, oily substance.

¹H-NMR (CDCl₃) δ: 3.79 (3H, s), 4.11 (2H, s), 6.37 (1H, s), 6.80 (1H, dd, J=8.1, 2.4 Hz), 6.86-6.92 (2H, m), 7.03 (1H, t, J=7.5 Hz), 7.20-7.27 (1H, m), 7.32-7.38 (2H, m).

### Reference Example 45

### 3-[2-(3-Methoxybenzyl)-1-benzofuran-7-yl]benzoic acid

7-Bromo-2-(3-methoxybenzyl)-1-benzofuran obtained in Reference Example 44 was used in the same manner as in Reference Example 10 to obtain the titled compound. Yield: 57%; melting point: 127-129° C. (ethyl acetate-hexane).

¹H-NMR (CDCl₃) δ: 3.78 (3H, s), 4.11 (2H, s), 6.43 (1H, s), 6.78-7.00 (3H, m), 7.22-7.30 (2H, m), 7.40-7.49 (2H, m), 7.59 (1H, t, J=7.8 Hz), 8.12 (2H, t, J=7.8 Hz), 8.63 (1H, s), 1H unconfirmed.

### Reference Example 46

### (7-Bromo-1-benzofuran-2-yl)(3,4-dimethoxyphenyl)methanone

3-Bromo-2-hydroxybenzaldehyde and 2-bromo-1-(3,4-dimethoxyphenyl)ethanone were used in the same manner as in Reference Example 8 to obtain the titled compound. Yield: 78%; melting point: 137-138° C. (methanol).

¹H-NMR (CDCl₃) δ: 4.00 (3H, s), 4.02 (3H, s), 7.00 (1H, d, J=8.4 Hz), 7.17-7.25 (1H, m), 7.62-7.70 (3H, m), 7.86 (1H, s), 7.98 (1H, dt, J=8.4, 0.9 Hz).

### Reference Example 47

### 7-Bromo-2-(3,4-dimethoxybenzyl)-1-benzofuran

(7-Bromo-1-benzofuran-2-yl)(3,4-dimethoxyphenyl)methanone obtained in Reference Example 46 was used in the same manner as in Reference Example 9 to obtain the titled compound. 86% yield, oily substance.

¹H-NMR (CDCl₃) δ: 3.87 (6H, s), 4.08 (2H, s), 6.35 (1H, s), 6.78-6.90 (3H, m), 7.04 (1H, t, J=7.8 Hz), 7.31-7.42 (2H, m).

### Reference Example 48

### 3-[2-(3,4-dimethoxybenzyl)-1-benzofuran-7-yl]benzoic acid

7-Bromo-2-(3,4-dimethoxybenzyl)-1-benzofuran obtained in Reference Example 47 was used in the same manner as in Reference Example 10 to obtain the titled compound. Yield: 67%; melting point: 122-123° C. (ethyl acetate-hexane).

¹H-NMR (CDCl₃) δ: 3.85 (3H, s), 3.87 (3H, s), 4.07 (2H, s), 6.40 (1H, s), 6.82-6.94 (3H, m), 7.27 (1H, t, J=7.8 Hz), 7.41 (1H, dd, J=7.5, 1.5 Hz), 7.47 (1H, dd, J=7.8, 1.5 Hz), 7.58 (1H, t, J=7.8 Hz), 8.07-8.15 (2H, m), 8.63 (1H, d, J=1.8 Hz), 1H unconfirmed.

### Reference Example 49

### 7-Bromo-1-[3-(trifluoromethyl)benzyl]-1H-indole

7-Bromoindole (2.0 g, 10.2 mmol) was added to a mixture of ground potassium hydroxide (2.29 g, 15.3 mmol) and DMSO (20 mL), and the mixture was stirred for 30 min. 3-(trifluoromethyl)benzyl bromide (2.34 mL, 15.3 mmol) was then added dropwise, and the mixture was stirred for 1 hour. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane-ethyl acetate=10:1) to give 3.4 g of the titled compound (yield 94%) in the form of an oily substance.

¹H-NMR (CDCl₃) δ: 5.86 (2H, s), 6.59 (1H, d, J=3.3 Hz), 6.96 (1H, t, J=7.6 Hz), 7.06 (1H, d, J=7.8 Hz), 7.10 (1H, d, J=3.3 Hz), 7.30-7.45 (3H, m), 7.50 (1H, d, J=7.5 Hz), 7.59 (1H, d, J=7.5 Hz).

### Reference Example 50

### 7-Bromo-2-[3-(trifluoromethyl)benzyl]-1H-indole

A mixture of the 7-bromo-1-[3-(trifluoromethyl)benzyl]-1H-indole (3.2 g, 9.04 mmol) obtained in Reference Example 49 and polyphosphoric acid (15 g) was stirred for 1 hour at 85 to 90° C. Water was poured into the mixture, and the mixture was extracted with ethyl acetate. The extract was washed with saturated aqueous sodium bicarbonate and dried over anhydrous magnesium sulfate, and the solvent was then distilled off at reduced pressure. The residue was purified by silica gel column chromatography (hexane-ethyl acetate 20:1→10:1) to give 1.87 g of the titled compound (yield 58%) in the form of a crude oily substance. The product was used, without further purification, in the following reaction.

### Reference Example 51

### Ethyl 3-[2-[3-(trifluoromethyl)benzyl]-1H-indol-7-yl]benzoate

7-Bromo-2-[3-(trifluoromethyl)benzyl]-1H-indole obtained in Reference Example 50 and [3-(ethoxycarbonyl)phenyl]boronic acid were used in the same manner as in Reference Example 4 to obtain the titled compound in the form of an oily substance. Yield: 41%.

¹H-NMR (CDCl₃) δ: 1.40 (3H, t, J=7.2 Hz), 4.40 (2H, q, J=7.2 Hz), 5.44 (2H, s), 7.10-7.25 (2H, m), 7.35-7.60 (6H, m), 7.70-7.80 (2H, m), 7.97 (1H, d, J=7.8 Hz), 8.04 (1H, d, J=7.8 Hz), 8.27 (1H, s).

### Reference Example 52

### 7-Bromo-2-[3-(trifluoromethyl)benzyl]-2H-indazole

A DMF (2.0 mL) solution of 7-bromo-1H-indazole (1.00 g, 5.08 mmol) and 1-(bromomethyl)-3-(trifluoromethyl)benzene (1.16 mL, 7.61 mmol) was stirred for 20 hours at 50° C. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate-hexane=1:9) to give 983 mg of the titled compound (yield 55%) in the form of an oily substance.

¹H-NMR (CDCl₃) δ: 5.72 (2H, s), 6.91-7.00 (1H, m), 7.42-7.56 (3H, m), 7.56-7.65 (3H, m), 7.96 (1H, s).

### Reference Example 53

### Ethyl 3-[2-[3-(trifluoromethyl)benzyl]-2H-indazol-7-yl]benzoate

7-Bromo-2-[3-(trifluoromethyl)benzyl]-2H-indazole obtained in Reference Example 52 was used in the same manner as in Reference Example 4 to obtain the titled compound. 86% yield, oily substance.

¹H-NMR (CDCl₃) δ: 1.39 (3H, t, J=7.2 Hz), 4.40 (2H, q, J=7.2 Hz), 5.69 (2H, s), 7.15-7.24 (1H, m), 7.45-7.68 (7H, m), 7.98 (1H, s), 8.02-8.08 (1H, m), 8.26-8.32 (1H, m), 8.66-8.69 (1H, m).

### Reference Example 54

### 3-[2-[3-(Trifluoromethyl)benzyl]-2H-indazol-7-yl]benzoic acid

Ethyl 3-[2-[3-(trifluoromethyl)benzyl]-2H-indazol-7-yl]benzoate obtained in Reference Example 53 was used in the same manner as in Reference Example 5 to obtain the titled compound in solid form. Yield: 75%.

¹H-NMR (DMSO-d₆) δ: 5.83 (2H, s), 7.13-7.24 (1H, m), 7.48-7.82 (7H, m), 7.91-7.97 (1H, m), 8.25-8.31 (1H, m), 8.61-8.68 (2H, m).

### Reference Example 55

### 4-Bromo-2-[3-(trifluoromethyl)benzyl]-2H-indazole

4-Bromo-1H-indazole and 1-(bromomethyl)-3-(trifluoromethyl)benzene were used in the same manner as in Reference Example 52 to obtain the titled compound in the form of an oily substance. Yield: 58%.

¹H-NMR (CDCl₃) δ: 5.64 (2H, s), 7.12-7.19 (1H, m), 7.22-7.28 (1H, m), 7.42-7.54 (2H, m), 7.57-7.70 (3H, m), 7.97 (1H, s).

### Reference Example 56

### Ethyl 3-[2-[3-(trifluoromethyl)benzyl]-2H-indazol-4-yl]benzoate

4-Bromo-2-[3-(trifluoromethyl)benzyl]-2H-indazole obtained in Reference Example 55 and [3-(ethoxycarbonyl)phenyl]boronic acid were used in the same manner as in Reference Example 4 to obtain the titled compound in the form of an oily substance. Yield: 88%.

¹H-NMR (CDCl₃) δ: 1.40 (3H, t, J=7.2 Hz), 4.41 (2H, q, J=7.2 Hz), 5.68 (2H, s), 7.19-7.25 (1H, m), 7.35-7.61 (6H, m), 7.75 (1H, d, J=8.9 Hz), 7.81-7.86 (1H, m), 8.02-8.14 (2H, m), 8.34 (1H, t, J=1.6 Hz).

### Reference Example 57

### 3-[2-[3-(Trifluoromethyl)benzyl]-2H-indazol-4-yl]benzoic acid

Ethyl 3-[2-[3-(trifluoromethyl)benzyl]-2H-indazol-4-yl]benzoate obtained in Reference Example 56 was used in the same manner as in Reference Example 5 to obtain the titled compound in solid form. Yield: 83%.

¹H-NMR (DMSO-d₆) δ: 5.80 (2H, s), 7.23 (1H, d, J=6.4 Hz), 7.37 (1H, dd, J=8.7, 6.4 Hz), 7.54-7.72 (5H, m), 7.79 (1H, s), 7.95-8.04 (2H, m), 8.24 (1H, t, J=1.6 Hz), 8.78 (1H, d, J=0.8 Hz).

### Reference Example 58

### 7-Bromo-3-methyl-1H-indazole

A mixture of 1-(3-bromo-2-fluorophenyl)ethanone (1.85 g, 8.52 mmol) and hydrazine monohydrate (12 mL) was stirred for 2 hours at 120° C. Ice water was poured into the reaction solution, and the mixture was extracted with ethyl acetate. The extract was washed with water and dried over anhydrous magnesium sulfate, and the solvent was distilled off at reduced pressure. Diethyl ether and hexane were added to the residue, and 1.04 g of the titled compound (yield 58%) was filtered off in solid form.

¹H-NMR (CDCl₃) δ: 2.59 (3H, s), 6.99-7.08 (1H, m), 7.52 (1H, dd, J=7.5, 0.8 Hz), 7.63 (1H, dd, J=8.1, 0.8 Hz), 9.85 (1H, br s).

### Reference Example 59

### Ethyl 3-(3-methyl-1H-indazol-7-yl]benzoate

7-Bromo-3-methyl-1H-indazole obtained in Reference Example 58 and [3-(ethoxycarbonyl)phenyl]boronic acid were used in the same manner as in Reference Example 4 to obtain the titled compound in the form of an oily substance. Yield: 47%.

¹H-NMR (CDCl₃) δ: 1.41 (3H, t, J=7.2 Hz), 2.63 (3H, s), 4.42 (2H, q, J=7.2 Hz), 7.22-7.30 (1H, m), 7.41-7.49 (1H, m), 7.61 (1H, t, J=7.8 Hz), 7.70 (1H, d, J=7.8 Hz), 7.81-7.87 (1H, m), 8.06-8.14 (1H, m), 8.32-8.36 (1H, m), 10.14 (1H, br s).

### Reference Example 60

### Ethyl 3-[3-methyl-2-[3-(trifluoromethyl)benzyl]-2H-indazol-7-yl]benzoate

A DMF (3.6 mL) solution of ethyl 3-(3-methyl-1H-indazol-7-yl]benzoate (180 mg, 0.642 mmol) obtained in Reference Example 59 and 1-(bromomethyl)-3-(trifluoromethyl)benzene (0.147 mL, 0.963 mmol) was stirred over night at 70° C. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate-hexane=3:7) to give 177 mg of the titled compound (yield 63%) in solid form.

¹H-NMR (CDCl₃) δ: 1.38 (3H, t, J=7.0 Hz), 2.57 (3H, s), 4.40 (2H, q, J=7.0 Hz), 5.68 (2H, s), 7.13-7.20 (1H, m), 7.27-7.33 (1H, m), 7.39-7.60 (6H, m), 8.01-8.07 (1H, m), 8.28-8.36 (1H, m), 8.66-8.70 (1H, m).

### Reference Example 61

### 3-[3-Methyl-2-[3-(trifluoromethyl)benzyl]-2H-indazol-7-yl]benzoic acid

Ethyl 3-[3-methyl-2-[3-(trifluoromethyl)benzyl]-2H-indazol-7-yl]benzoate obtained in Reference Example 60 was used in the same manner as in Reference Example 5 to obtain the titled compound in solid form. Yield: 80%.

¹H-NMR (DMSO-d₆) δ: 2.65 (3H, s), 5.81 (2H, s), 7.14 (1H, dd, J=8.3, 7.0 Hz), 7.40-7.76 (7H, m), 7.89-7.96 (1H, m), 8.29-8.36 (1H, m), 8.66 (1H, t, J=1.6 Hz).

### Reference Example 62

### 4-Bromo-2-(3-methoxybenzyl)-2H-indazole

4-Bromo-1H-indazole and 1-(bromomethyl)-3-methoxybenzene were used in the same manner as in Reference Example 52 to obtain the titled compound in the form of an oily substance. Yield: 53%.

¹H-NMR (CDCl₃) δ: 3.78 (3H, m), 5.56 (2H, s), 6.80-6.92 (3H, m), 7.10-7.17 (1H, m), 7.19-7.33 (2H, m), 7.66 (1H, d, J=8.5 Hz), 7.92 (1H, d, J=0.8 Hz).

### Reference Example 63

### Ethyl 3-[2-(3-methoxybenzyl)-2H-indazol-4-yl]benzoate

4-Bromo-2-(3-methoxybenzyl)-2H-indazole obtained in Reference Example 62 and [3-(ethoxycarbonyl)phenyl]boronic acid were used in the same manner as in Reference Example 4 to obtain the titled compound in solid form. Yield: 79%.

¹H-NMR (CDCl₃) δ: 1.40 (3H, t, J=7.2 Hz), 3.76 (3H, s), 4.40 (2H, q, J=7.2 Hz), 5.59 (2H, s), 6.79-6.90 (3H, m), 7.17-7.29 (2H, m), 7.34-7.42 (1H, m), 7.54 (1H, t, J=7.8 Hz), 7.75 (1H, d, J=8.9 Hz), 7.79-7.85 (1H, m), 8.01-8.08 (2H, m), 8.33 (1H, t, J=1.5 Hz).

### Reference Example 64

### 3-[2-(3-Methoxybenzyl)-2H-indazol-4-yl]benzoic acid

Ethyl 3-[2-(3-methoxybenzyl)-2H-indazol-4-yl]benzoate obtained in Reference Example 63 was used in the same manner as in Reference Example 5 to obtain the titled compound in solid form. Yield: 88%.

¹H-NMR (DMSO-d₆) δ: 3.72 (3H, s), 5.64 (2H, s), 6.82-6.99 (3H, m), 7.18-7.30 (2H, m), 7.36 (1H, dd, J=8.6, 6.9 Hz), 7.60-7.71 (2H, m), 7.93-8.03 (2H, m), 8.22-8.26 (1H, m), 8.69 (1H, s).

### Reference Example 65

### 4-Bromo-2-(2-fluorobenzyl)-2H-indazole

4-Bromo-1H-indazole and 1-(bromomethyl)-2-fluorobenzene were used in the same manner as in Reference Example 52 to obtain the titled compound in the form of an oily substance. Yield: 63%.

¹H-NMR (CDCl₃) δ: 5.65 (2H, s), 7.07-7.17 (3H, m), 7.20-7.39 (3H, m), 7.65 (1H, d, J=8.5 Hz), 7.98 (1H, s).

### Reference Example 66

### Ethyl 3-[2-(2-fluorobenzyl)-2H-indazol-4-yl]benzoate

4-Bromo-2-(2-fluorobenzyl)-2H-indazole obtained in Reference Example 65 and [3-(ethoxycarbonyl)phenyl]boronic acid were used in the same manner as in Reference Example 4 to obtain the titled compound in solid form. Yield: 64%.

¹H-NMR (CDCl₃) δ: 1.41 (3H, t, J=7.2 Hz), 4.41 (2H, q, J=7.2 Hz), 5.68 (2H, s), 7.03-7.14 (2H, m), 7.17-7.43 (4H, m), 7.55 (1H, t, J=7.5 Hz), 7.74 (1H, d, J=8.7 Hz), 7.79-7.87 (1H, m), 8.02-8.10 (1H, m), 8.12 (1H, s), 8.34 (1H, t, J=1.6 Hz).

### Reference Example 67

### 3-[2-(2-Fluorobenzyl)-2H-indazol-4-yl]benzoic acid

Ethyl 3-[2-(2-fluorobenzyl)-2H-indazol-4-yl]benzoate obtained in Reference Example 66 was used in the same manner as in Reference Example 5 to obtain the titled compound in solid form. Yield: 85%.

¹H-NMR (DMSO-d₆) δ: 5.76 (2H, s), 7.08-7.47 (6H, m), 7.58-7.74 (2H, m), 7.92-8.04 (2H, m), 8.23 (1H, t, J=1.7 Hz), 8.69 (1H, s).

### Reference Example 68

### 4-Bromo-2-(3-fluorobenzyl)-2H-indazole

4-Bromo-1H-indazole and 1-(bromomethyl)-3-fluorobenzene were used in the same manner as in Reference Example 52 to obtain the titled compound in the form of an oily substance. Yield: 67%.

¹H-NMR (CDCl₃) δ: 5.58 (2H, s), 6.92-7.10 (3H, m), 7.11-7.19 (1H, m), 7.21-7.28 (1H, m), 7.29-7.38 (1H, m), 7.66 (1H, d, J=8.7 Hz), 7.95 (1H, d, J=0.9 Hz).

### Reference Example 69

### Ethyl 3-[2-(3-fluorobenzyl)-2H-indazol-4-yl]benzoate

4-Bromo-2-(3-fluorobenzyl)-2H-indazole obtained in Reference Example 68 and [3-(ethoxycarbonyl)phenyl]boronic acid were used in the same manner as in Reference Example 4 to obtain the titled compound in solid form. Yield: 97%.

¹H-NMR (CDCl₃) δ: 1.40 (3H, t, J=7.2 Hz), 4.41 (2H, q, J=7.2 Hz), 5.61 (2H, s), 6.92-7.09 (3H, m), 7.17-7.43 (3H, m), 7.50-7.58 (1H, m), 7.71-7.78 (1H, m), 7.80-7.85 (1H, m), 8.02-8.10 (2H, m), 8.34 (1H, t, J=1.6 Hz).

### Reference Example 70

### 3-[2-(3-Fluorobenzyl)-2H-indazol-4-yl]benzoic acid

Ethyl 3-[2-(3-fluorobenzyl)-2H-indazol-4-yl]benzoate obtained in Reference Example 69 was used in the same manner as in Reference Example 5 to obtain the titled compound in solid form. Yield: 85%.

¹H-NMR (DMSO-d₆) δ: 5.71 (2H, s), 7.08.-7.26 (4H, m), 7.31-7.45 (2H, m), 7.61-7.71 (2H, m), 7.95-8.04 (2H, m), 8.24 (1H, t, J=1.6 Hz), 8.73 (1H, d, J=0.8 Hz).

### Reference Example 71

### 4-Bromo-2-(4-fluorobenzyl)-2H-indazole

4-Bromo-1H-indazole and 1-(bromomethyl)-4-fluorobenzene were used in the same manner as in Reference Example 52 to obtain the titled compound in the form of an oily substance. Yield: 80%.

¹H-NMR (CDCl₃) δ: 5.56 (2H, s), 7.00-7.20 (3H, m), 7.21-7.35 (3H, m), 7.66 (1H, d, J=8.7 Hz), 7.91 (1H, s).

### Reference Example 72

### Ethyl 3-[2-(4-fluorobenzyl)-2H-indazol-4-yl]benzoate

4-Bromo-2-(4-fluorobenzyl)-2H-indazole obtained in Reference Example 71 and [3-(ethoxycarbonyl)phenyl]boronic acid were used in the same manner as in Reference Example 4 to obtain the titled compound in solid form. Yield: 93%.

¹H-NMR (CDCl₃) δ: 1.40 (3H, t, J=7.1 Hz), 4.41 (2H, q, J=7.1 Hz), 5.59 (2H, s), 6.97-7.07 (2H, m), 7.20 (1H, dd, J=7.0, 0.8 Hz), 7.23-7.32 (2H, m), 7.38 (1H, dd, J=8.8, 7.0 Hz), 7.54 (1H, t, J=7.0 Hz), 7.74 (1H, d, J=8.8 Hz), 7.78-7.85 (1H, m), 8.01-8.09 (2H, m), 8.33 (1H, t, J=1.6 Hz).

### Reference Example 73

### 3-[2-(4-Fluorobenzyl)-2H-indazol-4-yl]benzoic acid

Ethyl 3-[2-(4-fluorobenzyl)-2H-indazol-4-yl]benzoate obtained in Reference Example 72 was used in the same manner as in Reference Example 5 to obtain the titled compound in solid form. Yield: 79%.

¹H-NMR (DMSO-d₆) δ: 5.67 (2H, s), 7.09-7.27 (3H, m), 7.29-7.48 (3H, m), 7.59-7.73 (2H, m), 7.93-8.06 (2H, m), 8.23 (1H, t, J=1.6 Hz), 8.70 (1H, s).

### Reference Example 74

### 4-Bromo-2-(3-chloro-4-fluorobenzyl)-2H-indazole

4-Bromo-1H-indazole and 4-(bromomethyl)-2-chloro-1-fluorobenzene were used in the same manner as in Reference Example 52 to obtain the titled compound in the form of an oily substance. Yield: 78%.

¹H-NMR (CDCl₃) δ: 5.53 (2H, s), 7.07-7.21 (3H, m), 7.22-7.29 (1H, m), 7.37 (1H, dd J=6.6, 1.9 Hz), 7.61-7.70 (1H, m), 7.94 (1H, d, J=0.8 Hz).

### Reference Example 75

### Ethyl 3-[2-(3-chloro-4-fluorobenzyl)-2H-indazol-4-yl]benzoate

4-Bromo-2-(3-chloro-4-fluorobenzyl)-2H-indazole obtained in Reference Example 74 and [3-(ethoxycarbonyl)phenyl]boronic acid were used in the same manner as in Reference Example 4 to obtain the titled compound in solid form. Yield: 84%.

¹H-NMR (CDCl₃) δ: 1.41 (3H, t, J=7.2 Hz), 4.41 (2H, q, J=7.2 Hz), 5.56 (2H, s), 7.06-7.24 (3H, m), 7.31-7.45 (2H, m), 7.55 (1H, t, J=7.7 Hz), 7.74 (1H, d, J=8.7 Hz), 7.79-7.85 (1H, m), 8.04-8.10 (2H, m), 8.34 (1H, t, J=1.6 Hz).

### Reference Example 76

### 3-[2-(3-Chloro-4-fluorobenzyl)-2H-indazol-4-yl]benzoic acid

Ethyl 3-[2-(3-chloro-4-fluorobenzyl)-2H-indazol-4-yl]benzoate obtained in Reference Example 75 was used in the same manner as in Reference Example 5 to obtain the titled compound in solid form. Yield: 98%.

¹H-NMR (DMSO-d₆) δ: 5.68 (2H, s), 7.23 (1H, d, J=6.4 Hz), 7.32-7.42 (3H, m), 7.58-7.72 (3H, m), 7.93-8.03 (2H, m), 8.24 (1H, t, J=1.6 Hz), 8.73 (1H, s).

### Reference Example 77

### 4-Bromo-2-(3-chlorobenzyl)-2H-indazole

4-Bromo-1H-indazole and 1-(bromomethyl)-3-chlorobenzene were used in the same manner as in Reference Example 52 to obtain the titled compound in the form of an oily substance. Yield: 68%.

¹H-NMR (CDCl₃) δ: 5.56 (2H, s), 7.11-7.20 (2H, m), 7.22-7.35 (4H, m), 7.66 (1H, d, J=8.7 Hz), 7.95 (1H, d, J=0.8 Hz).

### Reference Example 78

### Ethyl 3-[2-(3-chlorobenzyl)-2H-indazol-4-yl]benzoate

4-Bromo-2-(3-chlorobenzyl)-2H-indazole obtained in Reference Example 77 and [3-(ethoxycarbonyl)phenyl]boronic acid were used in the same manner as in Reference Example 4 to obtain the titled compound in solid form. Yield: 81%.

¹H-NMR (CDCl₃) δ: 1.40 (3H, t, J=7.2 Hz), 4.41 (2H, q, J=7.2 Hz), 5.59 (2H, s), 7.11-7.19 (1H, m), 7.19-7.32 (4H, m), 7.35-7.43 (1H, m), 7.55 (1H, t, J=7.7 Hz), 7.75 (1H, d, J=8.9 Hz), 7.79-7.86 (1H, m), 8.02-8.11 (2H, m), 8.34 (1H, t, J=1.5 Hz).

### Reference Example 79

### 3-[2-(3-Chlorobenzyl)-2H-indazol-4-yl]benzoic acid

Ethyl 3-[2-(3-chlorobenzyl)-2H-indazol-4-yl]benzoate obtained in Reference Example 78 was used in the same manner as in Reference Example 5 to obtain the titled compound in solid form. Yield: 82%.

¹H-NMR (DMSO-d₆) δ: 5.70 (2H, s), 7.17-7.50 (6H, m), 7.58-7.75 (2H, m), 7.94-8.06 (2H, m), 8.24 (1H, t, J=1.7 Hz), 8.74 (1H, d, J=0.8 Hz).

### Reference Example 80

### 4-Bromo-2-(3,4-difluorobenzyl)-2H-indazole

4-Bromo-1H-indazole and 4-(bromomethyl)-1,2-difluorobenzene were used in the same manner as in Reference Example 52 to obtain the titled compound in the form of an oily substance. Yield: 77%.

¹H-NMR (CDCl₃) δ: 5.54 (2H, s), 7.01-7.29 (5H, m), 7.62-7.68 (1H, m), 7.95 (1H, d, J=0.8 Hz).

### Reference Example 81

### Ethyl 3-[2-(3,4-difluorobenzyl)-2H-indazol-4-yl]benzoate

4-Bromo-2-(3,4-difluorobenzyl)-2H-indazole obtained in Reference Example 80 and [3-(ethoxycarbonyl)phenyl]boronic acid were used in the same manner as in Reference Example 4 to obtain the titled compound in solid form. Yield: 86%.

¹H-NMR (CDCl₃) δ: 1.41 (3H, t, J=7.2 Hz), 4.41 (2H, q, J=7.2 Hz), 5.77 (2H, s), 6.97-7.16 (3H, m), 7.18-7.24 (1H, m), 7.40 (1H, dd, J=8.8, 6.9 Hz), 7.55 (1H, t, J=7.7 Hz), 7.74 (1H, d, J=8.8 Hz), 7.79-7.86 (1H, m), 8.01-8.10 (2H, m), 8.34 (1H, t, J=1.6 Hz).

### Reference Example 82

### 3-[2-(3,4-difluorobenzyl)-2H-indazol-4-yl]benzoic acid

Ethyl 3-[2-(3,4-difluorobenzyl)-2H-indazol-4-yl]benzoate obtained in Reference Example 81 was used in the same manner as in Reference Example 5 to obtain the titled compound in solid form. Yield: 77%.

¹H-NMR (DMSO-d₆) δ: 5.68 (2H, s), 7.16-7.27 (2H, m), 7.31-7.54 (3H, m), 7.61-7.71 (2H, m), 7.94-8.04 (2H, m), 8.24 (1H, t, J=1.6 Hz), 8.72 (1H, d, J=0.8 Hz), 13.09 (1H, br s).

### Reference Example 83

### 4-Bromo-2-(3-chloro-5-fluorobenzyl)-2H-indazole

4-Bromo-1H-indazole and 1-(bromomethyl)-3-chloro-5-fluorobenzene were used in the same manner as in Reference Example 52 to obtain the titled compound in solid form. Yield: 63%.

¹H-NMR (CDCl₃) δ: 5.55 (2H, s), 6.84-6.92 (1H, m), 7.02-7.10 (2H, m), 7.13-7.20 (1H, m), 7.23-7.30 (1H, m), 7.63-7.70 (1H, m), 7.98 (1H, s).

### Reference Example 84

### Ethyl 3-[2-(3-chloro-5-fluorobenzyl)-2H-indazol-4-yl]benzoate

4-Bromo-2-(3-chloro-5-fluorobenzyl)-2H-indazole obtained in Reference Example 83 and [3-(ethoxycarbonyl)phenyl]boronic acid were used in the same manner as in Reference Example 4 to obtain the titled compound in solid form. Yield: 90%.

¹H-NMR (CDCl₃) δ: 1.41 (3H, t, J=7.2 Hz), 4.41 (2H, q, J=7.2 Hz), 5.58 (2H, s), 6.82-6.90 (1H, m), 7.00-7.08 (2H, m), 7.19-7.28 (1H, m), 7.41 (1H, dd, J=8.7, 7.0 Hz), 7.52-7.60 (1H, m), 7.75 (1H, d, J=8.7 Hz), 7.81-7.87 (1H, m), 8.04-8.12 (2H, m), 8.35 (1H, t, J=1.6 Hz).

### Reference Example 85

### 3-[2-(3-Chloro-5-fluorobenzyl)-2H-indazol-4-yl]benzoic acid

Ethyl 3-[2-(3-chloro-5-fluorobenzyl)-2H-indazol-4-yl]benzoate obtained in Reference Example 84 was used in the same manner as in Reference Example 5 to obtain the titled compound in solid form. Yield: 74%.

¹H-NMR (DMSO-d₆) δ: 5.71 (2H, s), 7.17-7.43 (5H, m), 7.62-7.72 (2H, m), 7.95-8.05 (2H, m), 8.24 (1H, t, J=1.6 Hz), 8.75 (1H, d, J=0.8 Hz), 13.12 (1H, br s).

### Reference Example 86

### 2-Benzyl-4-bromo-2H-indazole

4-Bromo-1H-indazole and (bromomethyl)benzene were used in the same manner as in Reference Example 52 to obtain the titled compound in solid form. Yield: 75%.

¹H-NMR (CDCl₃) δ: 5.59 (2H, s), 7.10-7.17 (1H, m), 7.21-7.27 (1H, m), 7.28-7.42 (5H, m), 7.63-7.70 (1H, m), 7.91 (1H, s).

### Reference Example 87

### Ethyl 3-(2-benzyl-2H-indazol-4-yl]benzoate

2-Benzyl-4-bromo-2H-indazole obtained in Reference Example 86 and [3-(ethoxycarbonyl)phenyl]boronic acid were used in the same manner as in Reference Example 4 to obtain the titled compound in solid form. Yield: 85%.

¹H-NMR (CDCl₃) δ: 1.40 (3H, t, J=7.2 Hz), 4.40 (2H, q, J=7.2 Hz), 5.63 (2H, s), 7.17-7.22 (1H, m), 7.25-7.42 (6H, m), 7.49-7.57 (1H, m), 7.75 (1H, d, J=8.7 Hz), 7.79-7.85 (1H, m), 8.01-8.08 (2H, m), 8.33 (1H, t, J=1.5 Hz).

### Reference Example 88

### 3-(2-Benzyl-2H-indazol-4-yl]benzoic acid

Ethyl 3-(2-benzyl-2H-indazol-4-yl]benzoate obtained in Reference Example 87 was used in the same manner as in Reference Example 5 to obtain the titled compound in solid form. Yield: 77%.

¹H-NMR (DMSO-d₆) δ: 5.68 (2H, s), 7.16-7.42 (7H, m), 7.60-7.71 (2H, m), 7.93-8.03 (2H, m), 8.23 (1H, t, J=1.5 Hz), 8.70 (1H, d, J=0.8 Hz), 13.09 (1H, br s).

### Reference Example 89

### 4-Bromo-2-[(6-methoxypyridin-2-yl)methyl]-2H-indazole

4-Bromo-1H-indazole and 2-(bromomethyl)-6-methoxypyridine were used in the same manner as in Reference Example 52 to obtain the titled compound in solid form. Yield: 25%.

¹H-NMR (CDCl₃) δ: 3.91 (3H, s), 5.60 (2H, s), 6.63-6.73 (2H, m), 7.09-7.19 (1H, m), 7.21-7.28 (1H, m), 7.47-7.56 (1H, m), 7.66 (1H, d, J=8.7 Hz), 8.14 (1H, s).

### Reference Example 90

### Ethyl 3-[2-[(6-methoxypyridin-2-yl)methyl]-2H-indazol-4-yl]benzoate

4-Bromo-2-[(6-methoxypyridin-2-yl)methyl]-2H-indazole obtained in Reference Example 89 and [3-(ethoxycarbonyl)phenyl]boronic acid were used in the same manner as in Reference Example 4 to obtain the titled compound in the form of an oily substance. Yield: 87%.

¹H-NMR (CDCl₃) δ: 1.41 (3H, t, J=7.2 Hz), 3.89 (3H, s), 4.41 (2H, q, J=7.2 Hz), 5.63 (2H, s), 6.63-6.69 (2H, m), 7.18-7.24 (1H, m), 7.39 (1H, dd, J=8.7, 7.0 Hz), 7.46-7.59 (2H, m), 7.75 (1H, d, J=8.7 Hz), 7.83-7.89 (1H, m), 8.03-8.10 (1H, m), 8.27 (1H, d, J=0.9 Hz), 8.37 (1H, t, J=1.6 Hz).

### Reference Example 91

### 3-[2-[(6-Methoxypyridin-2-yl)methyl]-2H-indazol-4-yl]benzoic acid

Ethyl 3-[2-[(6-methoxypyridin-2-yl)methyl]-2H-indazol-4-yl]benzoate obtained in Reference Example 90 was used in the same manner as in Reference Example 5 to obtain the titled compound in solid form. Yield: 66%.

¹H-NMR (DMSO-d₆) δ: 3.80 (3H, s), 5.70 (2H, s), 6.67-6.80 (2H, m), 7.24 (1H, d, J=6.8 Hz), 7.37 (1H, t, J=6.8 Hz), 7.58-7.74 (3H, m), 7.93-8.04 (2H, m), 8.27 (1H, s), 8.71 (1H, s), 13.12 (1H, br s).

### Reference Example 92

### 4-(4,4,5,5-tetramethyl-1,3,2-dioxaborolan-2-yl)-2-[3-(trifluoromethyl)benzyl]-2H-indazole

4-Bromo-2-[3-(trifluoromethyl)benzyl]-2H-indazole obtained in Reference Example 55 was used in the same manner as in Reference Example 19 to obtain the titled compound in solid form.

¹H-NMR (CDCl₃) δ: 1.37 (12H, s), 5.67 (2H, s), 7.32 (1H, dd, J=8.8, 6.5 Hz), 7.38-7.49 (2H, m), 7.54-7.61 (2H, m), 7.65 (1H, dd, J=6.5, 0.9 Hz), 7.83 (1H, d, J=8.8 Hz), 8.33 (1H, d, J=0.9 Hz).

### Reference Example 93

### 6-[2-[3-(Trifluoromethyl)benzyl]-2H-indazol-4-yl]pyridine-2-carboxylic acid

A mixture of 4-(4,4,5,5-tetramethyl-1,3,2-dioxaborolan-2-y )-2-[3-(trifluoromethyl)benzyl]-2H-indazole (235 mg, 0.586 mmol) obtained in Reference Example 92, methyl 6-bromopyridine-2-carboxylate (115 mg, 0.532 mmol), and tetrakis(triphenylphosphine)palladium (0) (73.8 mg, 0.064 mmol) in 2 N sodium carbonate aqueous solution (0.8 mL)-1,2-dimethoxyethane (2.5 mL) was stirred over night at 95° C. in a nitrogen atmosphere. Water was poured into the reaction solution, and the mixture was extracted with ethyl acetate. The extract was washed with water and dried over anhydrous magnesium sulfate, and the solvent was distilled off at reduced pressure. Diethyl ether was added to the residue to give 147 mg of the titled compound (yield 69%) in solid form.

¹H-NMR (CDCl₃) δ: 5.75 (2H, s), 7.12-8.07 (10H, m), 9.30 (1H, br s).

### Reference Example 94

### Methyl 2-[2-[3-(trifluoromethyl)benzyl]-2H-indazol-4-yl]pyridine-2-carboxylate

4-(4,4,5,5-Tetramethyl-1,3,2-dioxaborolan-2-yl)-2-[3-(trifluoromethyl)benzyl]-2H-indazol e obtained in Reference Example 92 and methyl 2-bromopyridine-4-carboxylate were used in the same manner as in Reference Example 4 to obtain the titled compound in solid form. Yield: 54%.

¹H-NMR (CDCl₃) δ: 4.00 (3H, s), 5.71 (2H, s), 7.38-7.49 (3H, m), 7.53-7.64 (2H, m), 7.68-7.74 (1H, m), 7.78 (1H, dd, J=5.0, 1.4 Hz), 7.82-7.87 (1H, m), 8.43-8.46 (1H, m), 8.82 (1H, d, J=0.9 Hz), 8.88 (1H, dd, J=5.0, 0.8 Hz).

### Reference Example 95

### 2-[2-[3-(Trifluoromethyl)benzyl]-2H-indazol-4-yl]pyridine-4-carboxylic acid

Methyl 2-[2-[3-(trifluoromethyl)benzyl]-2H-indazol-4-yl]pyridine-2-carboxylate obtained in Reference Example 94 was used in the same manner as in Reference Example 5 to obtain the titled compound in solid form. Yield: 76%.

¹H-NMR (DMSO-d₆) δ: 5.86 (2H, s), 7.41 (1H, dd, J=8.7, 7.2 Hz), 7.55-7.84 (7H, m), 8.40 (1H, s), 8.94 (1H, dd, J=4.9, 0.8 Hz), 9.11 (1H, d, J=0.8 Hz), 13.79 (1H, br s).

### Reference Example 96

### Ethyl 5-[2-[3-(trifluoromethyl)benzyl]-2H-indazol-4-yl]pyridine-3-carboxylate

4-(4,4,5,5-Tetramethyl-1,3,2-dioxaborolan-2-yl)-2-[3-(trifluoromethyl)benzyl]-2H-indazole obtained in Reference Example 92 and ethyl 5-bromopyridine-3-carboxylate were used in the same manner as in Reference Example 4 to obtain the titled compound in the form of an oily substance. Yield: 100%.

¹H-NMR (CDCl₃) δ: 1.43 (3H, t, J=7.1 Hz), 4.45 (2H, q, J=7.1 Hz), 5.68 (2H, s), 7.21-7.28 (1H, m), 7.38-7.50 (3H, m), 7.55-7.63 (2H, m), 7.81 (1H, d, J=8.7 Hz), 8.09 (1H, s) 8.55 (1H, t, J=1.9 Hz), 9.07 (1H, d, J=1.9 Hz), 9.22 (1H, d, J=1.9 Hz).

### Reference Example 97

### 5-[2-[3-(Trifluoromethyl)benzyl]-2H-indazol-4-yl]pyridine-3-carboxylic acid

Ethyl 5-[2-[3-(trifluoromethyl)benzyl]-2H-indazol-4-yl]pyridine-3-carboxylate obtained in Reference Example 96 was used in the same manner as in Reference Example 5 to obtain the titled compound in solid form. Yield: 60%.

¹H-NMR (DMSO-d₆) δ: 5.80 (2H, s), 7.29-7.46 (2H, m), 7.53-7.75 (4H, m), 7.80 (1H, s), 8.51 (1H, t, J=2.2 Hz), 8.88 (1H, s), 9.12 (1H, d, J=2.2 Hz), 9.17 (1H, d, J=2.2 Hz), 13.59 (1H, br s).

### Reference Example 98

### Methyl 4-[2-[3-(trifluoromethyl)benzyl]-2H-indazol-4-yl]pyridine-2-carboxylate

4-(4,4,5,5-Tetramethyl-1,3,2-dioxaborolan-2-yl)-2-[3-(trifluoromethyl)benzyl]-2H-indazole obtained in Reference Example 92 and methyl 4-bromopyridine-2-carboxylate were used in the same manner as in Reference Example 4 to obtain the titled compound in the form of an oily substance. Yield: 44%.

¹H-NMR (CDCl₃) δ: 4.05 (3H, s), 5.70 (2H, s), 7.30-7.35 (1H, m), 7.39-7.52 (3H, m), 7.52-7.63 (2H, m), 7.74 (1H, dd, J=5.1, 1.7 Hz), 7.85 (1H, d, J=8.7 Hz), 8.16 (1H, s), 8.44 (1H, d, J=1.7 Hz), 8.83 (1H, d, J=5.1 Hz).

### Reference Example 99

### 4-[2-[3-(Trifluoromethyl)benzyl]-2H-indazol-4-yl]pyridine-2-carboxylic acid

Methyl 4-[2-[3-(trifluoromethyl)benzyl]-2H-indazol-4-yl]pyridine-2-carboxylate obtained in Reference Example 98 was used in the same manner as in Reference Example 5 to obtain the titled compound in solid form. Yield: 60%.

¹H-NMR (DMSO-d₆) δ: 5.82 (2H, s), 7.36-7.46 (2H, m), 7.53-7.72 (3H, m), 7.72-7.83 (2H, m), 7.99 (1H, dd, J=5.0, 1.8 Hz), 8.32 (1H, d, J=0.9 Hz), 8.83 (1H, d, J=5.0 Hz), 8.91 (1H, d, J=0.9 Hz), 13.32 (1H, br s).

### Reference Example 100

### 2-[3-(Trifluoromethyl)benzyl]-1,3-benzoxazol-4-ol

A mixture of 2-nitrobenzene-1,3-diol (2.0 g, 12.7 mmol), 10%-palladium carbon (50% moisture content, 200 mg), and ammonium formate (3.15 g, 50.0 mmol) in ethanol (10 mL) was heated to reflux for 2 hours. The solids were filtered off, and the filtrate was concentrated at reduced pressure. Water and ethyl acetate were added to the residue, the organic layer was separated, and the aqueous layer was extracted with ethyl acetate. The organic layer was washed with water and then dried over anhydrous sodium sulfate. The solvent was distilled off at reduced pressure to give 2-aminobenzene-1,3-diol. [3-(trifluoromethyl)phenyl]acetic acid (3.10 g, 15.2 mmol) was added to a DMF (50 mL) solution of this compound, WSC (2.68 g, 14.0 mmol), and HOBt (1.89 g, 14.0 mmol), and the mixture was stirred for 16 hours at room temperature. The addition of saturated sodium bicarbonate aqueous solution to the reaction solution was followed by extraction with ethyl acetate. The organic layer was washed with water, dried over anhydrous magnesium sulfate, and then concentrated at reduced pressure. The residue was purified by silica gel column chromatography to give 1.45 g of N-(2,6-dihydroxyphenyl)-2-[3-(trifluoromethyl)phenyl]acetamide. p-toluenesulfonic acid monohydrate (420 mg, 2.21 mmol) was added to a toluene (20 mL) solution of this compound, and the mixture was stirred for 16 hours at 110° C. The reaction solution was concentrated at reduced pressure, 1 N potassium carbonate aqueous solution was then added to make the aqueous layer alkaline, and the mixture was extracted with ethyl acetate. The organic layer was washed with water and saturated saline, then dried over anhydrous sodium sulfate, and concentrated at reduced pressure. The resulting residue was purified by silica gel column chromatography (ethyl acetate-hexane 1:9→5:5) to give 1.38 g of the titled compound (yield 37%). Melting point: 157-158° C. (ethyl acetate-hexane).

¹H-NMR (CDCl₃) δ: 4.39 (2H, s), 6.89 (1H, d, J=8.1 Hz), 7.03 (1H, d, J=8.1 Hz), 7.21 (1H, d, J=8.4 Hz), 7.42 (1H, t, J=7.8 Hz), 7.52 (1H, d, J=7.8 Hz), 7.58 (1H, d, J=7.8 Hz), 7.66 (1H, s), 9.33 (1H, s).

### Reference Example 101

### 2-[3-(Trifluoromethyl)benzyl]-1,3-benzoxazol-4-yl trifluoromethanesulfonate

2-[3-(Trifluoromethyl)benzyl]-1,3-benzoxazol-4-ol obtained in Reference Example 100 was used in the same manner as in Reference Example 14 to obtain the titled compound. 80% yield, oily substance.

¹H-NMR (CDCl₃) δ: 4.37 (2H, s), 7.26 (1H, d, J=8.4 Hz), 7.36 (1H, t, J=7.8 Hz), 7.42-7.61 (4H, m), 7.67 (1H, s).

### Reference Example 102

### 3-[2-[3-(Trifluoromethyl)benzyl]-1,3-benzoxazol-4-yl]benzoic acid

2-[3-(Trifluoromethyl)benzyl]-1,3-benzoxazol-4-yl trifluoromethanesulfonate obtained in Reference Example 101 was used in the same manner as in Reference Example 10 to obtain the titled compound. Yield: 53%; melting point: 165-166° C. (ethyl acetate-hexane).

¹H-NMR (CDCl₃) δ: 4.39 (2H, s), 7.36-7.65 (7H, m), 7.69 (1H, s), 8.13 (1H, d, J=7.8 Hz), 8.23 (1H, d, J=7.8 Hz), 8.66 (1H, t, J=1.8 Hz), 1H unconfirmed.

### Reference Example 103

### 7-Bromo-2-[3-(trifluoromethyl)benzyl]-1,3-benzoxazole

An ethanol (40 mL)-water (5 mL) mixture of 2-bromo-6-nitrophenol (2.0 g, 9.17 mmol), iron (5.1 g, 91.7 mmol)), and ammonium chloride (245 mg, 4.59 mmol) was heated to 90° C. for 5 hours. The solids were filtered off, and the filtrate was concentrated at reduced pressure. The resulting residue was diluted with ethyl acetate, the solution was washed with water, saturated sodium bicarbonate aqueous solution, and saturated brine, then dried over anhydrous sodium sulfate, and concentrated at reduced pressure to give 1.7 g 2-amino-6-bromophenol crude product. [3-(trifluoromethyl)phenyl]acetic acid (2.25 g, 11.0 mmol) was added to a DMF (40 mL) solution of this compound, WSC (1.94 g, 10.1 mmol), and HOBt (1.36 g, 10.1 mmol), and the mixture was stirred for 16 hours at room temperature. The addition of saturated sodium bicarbonate aqueous solution to the reaction solution was followed by extraction with ethyl acetate. The organic layer was washed with water, dried over anhydrous magnesium sulfate, and then concentrated at reduced pressure. The residue was purified by silica gel column chromatography to give 1.31 g of N-(3-bromo-2-hydroxyphenyl)-2-[3-(trifluoromethyl)phenyl]acetamide. p-toluenesulfonic acid monohydrate (333 mg, 1.75 mmol) was added to a toluene (20 mL) solution of this compound, and the mixture was stirred for 16 hours at 110° C. The reaction solution was concentrated at reduced pressure, 1 N potassium carbonate aqueous solution was then added to make the aqueous layer alkaline, and it was extracted with ethyl acetate. The organic layer was washed with water and saturated brine, then dried over anhydrous sodium sulfate, and concentrated at reduced pressure. The resulting residue was purified by column chromatography (ethyl acetate-hexane 1:9→4:6) to give 1.05 g of the titled compound (yield 32%). Oily substance.

¹H-NMR (CDCl₃) δ: 4.40 (2H, s), 7.20 (1H, t, J=8.1 Hz), 7.43-7.68 (6H, m).

### Reference Example 104

### 3-[2-[3-(Trifluoromethyl)benzyl]-1,3-benzoxazol-7-yl]benzoic acid

7-Bromo-2-[3-(trifluoromethyl)benzyl]-1,3-benzoxazole obtained in Reference Example 103 was used in the same manner as in Reference Example 10 to obtain the titled compound. Yield: 32%; melting point: 175-176° C. (ethyl acetate-hexane).

¹H-NMR (CDCl₃) δ: 4.40 (2H, s), 7.38-7.75 (8H, m), 8.05 (1H, d, J=7.8 Hz), 8.16 (1H, d, J=7.8 Hz), 8.57 (1H, s), 1H unconfirmed.

### Reference Example 105

### 4-Methoxy-2-[3-(trifluoromethyl)benzyl]-1H-benzimidazole

[3-(Trifluoromethyl)phenyl]acetic acid (4.87 g, 23.9 mmol) was added to a DMF (50 mL) solution of 3-methoxybenzene-1,2-diamine (3.0 g, 21.7 mmol), WSC (4.87 g, 23.9 mmol), and HOBt (4.58 g, 23.9 mmol), and the mixture was stirred for 1.6 hours at room temperature. The addition of saturated sodium bicarbonate aqueous solution to the reaction solution was followed by extraction with ethyl acetate. The organic layer was washed with water, dried over anhydrous magnesium sulfate, and then concentrated at reduced pressure to give 1.45 g of an amide compound in the form of a crude product. 4 N hydrogen chloride-ethyl acetate solution (5.0 mL, 20 mmol) was added to an acetic acid (30 mL) solution of this compound, and the mixture was heated for 3 hours to 100° C. The reaction solution was concentrated at reduced pressure, 8 N sodium hydroxide aqueous solution was then added to neutralize the solution, and it was then extracted with ethyl acetate. The organic layer was washed with water and saturated brine, then dried over anhydrous sodium sulfate, and concentrated at reduced pressure. The residue was purified by silica gel column chromatography (ethyl acetate-hexane 10:90→60:40) to give 3.91 g of the titled compound (yield 59%).

¹H-NMR (CDCl₃) δ: 3.94 (3H, s), 4.30 (2H, s), 6.68 (1H, d, J=8.7 Hz), 6.90-7.20 (2H, m), 7.37-7.52 (4H, m), 9.5 (1H, br s).

### Reference Example 106

### 4-Methoxy-1-methyl-2-[3-(trifluoromethyl)benzyl]-1H-benzimidazole

In a nitrogen stream, sodium hydride (60% dispersion in mineral oil, 560 mg, 14.1 mmol) was added while cooled on ice to a THF (30 mL) solution of 4-methoxy-2-[3-(trifluoromethyl)benzyl]-1H-benzimidazole (3.91 g, 12.8 mmol) obtained in Reference Example 105, and the mixture was stirred for 30 min at room temperature. Iodomethane (2.0 g, 14.1 mmol) was added dropwise while cooled on ice to the mixture, and the reaction solution was stirred for 3 hours at room temperature. The addition of water was followed by extraction with ethyl acetate. The organic layer was washed with saturated brine, then dried over anhydrous sodium sulfate, and then concentrated at reduced pressure, and the residue was purified by silica gel column chromatography (ethyl acetate-hexane 5:95→35:65) to give 2.20 g of the titled compound (yield 54%).

¹H-NMR (CDCl₃) δ: 3.56 (3H, s), 4.03 (3H, s), 4.38 (2H, s), 6.70 (1H, d, J=7.8 Hz), 6.89 (1H, d, J=7.8 Hz), 7.19 (1H, t, J=7.8 Hz), 7.37-7.53 (4H, m).

### Reference Example 107

### 1-Methyl-2-[3-(trifluoromethyl)benzyl]-1H-benzimidazol-4-ol

4-Methoxy-1-methyl-2-[3-(trifluoromethyl)benzyl]-1H-benzimidazole obtained in Reference Example 106 was used in the same manner as in Reference Example 13 to obtain the titled compound.

¹H-NMR (CDCl₃) δ: 3.91 (3H, s), 4.14 (2H, s), 6.48 (1H, s), 6.62 (1H, d, J=7.8 Hz), 7.04 (1H, d, J=8.1 Hz), 7.15 (1H, t, J=7.8 Hz), 7.38-7.56 (4H, m).

### Reference Example 108

### 1-Methyl-2-[3-(trifluoromethyl)benzyl]-1H-benzimidazol-4-yl trifluoromethanesulfonate

1-Methyl-2-[3-(trifluoromethyl)benzyl]-1H-benzimidazol-4-ol obtained in Reference Example 107 was used in the same manner as in Reference Example 14 to obtain the titled compound.

¹H-NMR (CDCl₃) δ: 3.65 (3H, s), 4.42 (2H, s), 7.15-7.37 (3H, m), 7.40-7.59 (4H, m).

### Reference Example 109

### 3-[1-Methyl-2-[3-(trifluoromethyl)benzyl]-1H-benzimidazol-4-yl]benzoic acid

1-Methyl-2-[3-(trifluoromethyl)benzyl]-1H-benzimidazol-4-yl trifluoromethanesulfonate obtained in Reference Example 108 was used in the same manner as in Reference Example 10 to obtain the titled compound. Yield: 48%; melting point: 175-177° C. (ethyl acetate-hexane).

¹H-NMR (CDCl₃) δ: 3.63 (3H, s), 4.44 (2H, s), 7.23-7.57 (8H, m), 8.00-8.06 (1H, m), 8.23-8.28 (1H, m), 8.59 (1H, t, J=1.8 Hz), 1H unconfirmed.

### Reference Example 110

### 8-Bromo-2-[3-(trifluoromethyl)benzyl]imidazo[1,2-a]pyridine

A mixture of 3-bromopyridin-2-amine (1.11 g, 6.46 mmol), 1-bromo-3-[3-(trifluoromethyl)phenyl]propan-2-one (2.00 g, 7.11 mmol), and sodium bicarbonate (597 mg, 7.11 mmol) in ethanol (30 mL) was heated to reflux for 16 hours. The addition of water to the reaction solution was followed by extraction with ethyl acetate. The organic layer was washed with saturated brine, then dried over anhydrous sodium sulfate, and then concentrated at reduced pressure, and the residue was purified by silica gel column chromatography (ethyl acetate-hexane 10:90→50:50) to give 1.72 g of the titled compound (yield 75%) in the form of an oily substance.

¹H-NMR (CDCl₃) δ: 4.27 (2H, s), 6.62 (1H, t, J=6.9 Hz), 7.20 (1H, s), 7.37-7.59 (5H, m), 7.97 (1H, d, J=6.6 Hz).

### Reference Example 111

### 3-[2-[3-(Trifluoromethyl)benzyl]imidazo[1,2-a]pyridin-8-yl]benzoic acid

8-Bromo-2-[3-(trifluoromethyl)benzyl]imidazo[1,2-a]pyridine obtained in Reference Example 110 was used in the same manner as in Reference Example 10 to obtain the titled compound. Yield: 42%; melting point: 179-180° C. (ethyl acetate-hexane).

¹H-NMR (CDCl₃) δ: 4.32 (2H, s), 5.30 (1H, br s), 6.91 (1H, t, J=6.9 Hz), 7.18-7.28 (2H, m), 7.37-7.56 (4H, m), 7.60 (1H, s), 7.78-7.84 (1H, m), 7.87-7.91 (1H, m), 8.03 (1H, d, J=7.8 Hz), 8.57 (1H, t, J=1.8 Hz).

### Reference Example 112

### 3-Oxo-4-[3-(trifluoromethyl)phenyl]butanenitrile

Sodium hydride (207 mg, 5.17 mmol) was suspended in 1,4-dioxane (10 mL), drops of acetonitrile (271 μL, 5.17 mmol) were added to the suspension, and the suspension was stirred for 20 min at room temperature. A 1,4-dioxane (2 mL) solution of ethyl [3-(trifluoromethyl)phenyl]acetate (1.00 g, 4.31 mmol) was added to the reaction solution, and the mixture was heated to reflux for 3 hours. After cooling, the reaction solution was treated with the addition of water, 1 N hydrochloric acid was added to make the solution acidic, and the mixture was extracted with ethyl acetate. The organic layer was dried over anhydrous sodium sulfate and then concentrated at reduced pressure. The residue was purified by silica gel column chromatography to give 530 mg of the titled compound (yield 54%) in the form of crystals. Melting point: 82-83° C. (ethyl acetate-hexane).

¹H-NMR (CDCl₃) δ: 3.52 (2H, s), 3.98 (2H, s), 7.39-7.44 (1H, m), 7.47-7.56 (2H, m), 7.58-7.62 (1H, m).

### Reference Example 113

### 3-[3-(Trifluoromethyl)benzyl]-1H-pyrazol-5-amine

An ethanol (15 mL) solution of 3-oxo-4-[3-(trifluoromethyl)phenyl]butanenitrile (500 mg, 2.20 mmol) obtained in Reference Example 112 and hydrazine monohydrate (107 μL, 2.20 mmol) was heated to reflux for 4 hours. After cooling, the reaction solution was concentrated at reduced pressure and extracted with ethyl acetate. The organic layer was washed with saturated brine, dried over anhydrous sodium sulfate, and then concentrated at reduced pressure. The residue was purified by basic silica gel column chromatography (hexane-ethyl acetate 80:20→0:100) to give 310 mg of the titled compound (yield 58%) in the form of an oily substance.

¹H-NMR (CDCl₃) δ: 3.64 (2H, br s), 3.95 (2H, s), 5.46 (1H, s), 7.35-7.54 (4H, m).

### Reference Example 114

### 2-[3-(Trifluoromethyl)benzyl]pyrazolo[1,5-a]pyrimidin-7(4H)-one

An ethanol (3 mL) solution of 3-[3-(trifluoromethyl)benzyl]-1H-pyrazol-5-amine (300 mg, 1.24 mmol) obtained in Reference Example 113 and ethyl 3-(dimethylamino)acrylate (891 μL, 6.20 mmol) was heated to reflux for 2 days. After cooling, the reaction solution was concentrated at reduced pressure, and the resulting crystals were filtered off with the addition of ethyl acetate to give 110 mg of the titled compound (yield 30%) in the form of crystals. Melting point: 262-263° C.

¹H-NMR (DMSO-d₆) δ: 4.11 (2H, s), 5.64 (1H, d, J=7.5 Hz), 6.02 (1H, s), 7.50-7.63 (3H, m), 7.67 (1H, s), 7.80 (1H, d, J=7.3 Hz).

### Reference Example 115

### 7-Chloro-2-[3-(trifluoromethyl)benzyl]pyrazolo[1,5-a]pyrimidine

A phosphorus oxychloride (1 mL) solution of 2-[3-(trifluoromethyl)benzyl]pyrazolo[1,5-a]pyrimidin-7(4H)-one (100 mg, 0.34 mmol) obtained in Reference Example 114 and N,N-dimethylaniline (0.1 mL) was heated to reflux for 2 hours. After cooling, the reaction solution was concentrated at reduced pressure, and the residue was poured into ice. The resulting mixture was extracted with ethyl acetate, and the organic layer was dried over anhydrous sodium sulfate and then concentrated at reduced pressure. The residue was purified by silica gel column chromatography (hexane-ethyl acetate 100:0→80:20) to give 70 mg of the titled compound (yield 66%) in the form of an oily substance.

¹H-NMR (CDCl₃) δ: 4.32 (2H, s), 6.54 (1H, s), 6.94 (1H, d, J=4.5 Hz), 7.40-7.47 (1H, m), 7.48-7.54 (2H, m), 7.58 (1H, s), 8.34 (1H, d, J=4.5 Hz).

### Reference Example 116

### Ethyl 3-[2-[3-(trifluoromethyl)benzyl]pyrazolo[1,5-a]pyrimidin-7-yl]benzoate

7-Chloro-2-[3-(trifluoromethyl)benzyl]pyrazolo[1,5-a]pyrimidine obtained in Reference Example 115 and [3-(ethoxycarbonyl)phenyl]boronic acid were used in the same manner as in Reference Example 4 to obtain the titled compound. Yield: 48%. Amorphous solids.

¹H-NMR (CDCl₃) δ: 1.42 (3H, t, J=7.2 Hz), 4.26 (2H, s), 4.43 (2H, q, J=7.2 Hz), 6.53 (1H, s), 6.91 (1H, d, J=4.5 Hz), 7.43 (1H, d, J=7.6 Hz), 7.51 (2H, t, J=7.2 Hz), 7.62 (1H, s), 7.65 (1H, t, J=8.1 Hz), 8.24 (1H, d, J=8.0 Hz), 8.36 (1H, d, J=8.0 Hz), 8.50 (1H, d, J=4.2 Hz), 8.68 (1H, s).

### Reference Example 117

### 3-[2-[3-(Trifluoromethyl)benzyl]pyrazolo[1,5-a]pyrimidin-7-yl]benzoic acid

Ethyl 3-[2-[3-(trifluoromethyl)benzyl]pyrazolo[1,5-a]pyrimidin-7-yl]benzoate obtained in Reference Example 116 was used in the same manner as in Reference Example 5 to obtain the titled compound. Yield: 93%; melting point: 111-145° C. (ethyl acetate).

¹H-NMR (DMSO-d₆) δ: 4.28 (2H, s), 6.66 (1H, s), 7.26 (1H, d, J=4.5 Hz), 7.50-7.61 (2H, m), 7.63-7.69 (1H, m), 7.70-7.78 (2H, m), 8.13-8.19 (1H, m), 8.31-8.38 (1H, m), 8.58 (1H, d, J=4.3 Hz), 8.68 (1H, t, J=1.5 Hz), 13.22 (1H, br s).

### Reference Example 118

### 2-Chloro-8-methoxy[1,2,4]triazolo[1,5-a]pyridine

tert-Butyl nitrite (0.219 mL, 1.83 mmol) was added to an acetonitrile (4.0 mL) suspension of 8-methoxy[1,2,4]triazolo[1,5-a]pyridin-2-amine (200 mg, 1.22 mmol) and copper chloride (II) (197 mg, 1.46 mmol), and the suspension was stirred for 2 hours at 90° C. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate-hexane=1:1) to give 129 mg of the titled compound (yield 58%) in solid form.

¹H-NMR (CDCl₃) δ: 4.05 (3H, s), 6.81-6.88 (1H, m), 6.97 (1H, dd, J=7.9, 6.8 Hz), 8.15 (1H, dd, J=6.8, 0.9 Hz).

### Reference Example 119

### 8-Methoxy-N-[3-(trifluoromethyl)phenyl][1,2,4]triazolo[1,5-a]pyridin-2-amine

A mixture of 2-chloro-8-methoxy[1,2,4]triazolo[1,5-a]pyridine (100 mg, 0.545 mmol) obtained in Reference Example 118, 3-(trifluoromethyl)aniline (0.203 mL, 1.63 mmol), sodium tert-butoxide (78.5 mg, 0.817 mmol), Tris(dibenzylideneacetone)dipalladium (0) (10.0 mg, 0.011 mmol), and 2-(dicyclohexylphosphino)-2′,4′,6′-triisopropyl-1,1′-biphenyl (15.6 mg, 0.033 mmol) in 1,4-dioxane (2.0 mL) was stirred for 30 min at 120° C. in a microwave reactor (Initiator™, by Biotage). Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate-hexane=1:1) to give 147 mg of the titled compound (yield 88%) in solid form.

¹H-NMR (CDCl₃) δ: 3.99 (3H, s), 6.76-6.87 (2H, m), 7.21 (1H, d, J=8.0 Hz), 7.43 (1H, t, J=8.0 Hz), 7.82-7.91 (2H, m), 8.07-8.18 (2H, m).

### Reference Example 120

### 2-[[3-(Trifluoromethyl)phenyl]amino][1,2,4]triazolo[1,5-a]pyridin-8-yl trifluoromethanesulfonate

1.0 M boron tribromide/methylene chloride solution (0.908 mL) was added while cooled on ice to a methylene chloride (1.0 mL) solution of 8-methoxy-N-[3-(trifluoromethyl)phenyl][1,2,4]triazolo[1,5-a]pyridin-2-amine (140 mg, 0.454 mmol) obtained in Reference Example 119, and the mixture was heated to reflux for 5 hours. The reaction solution was poured into ice water and was extracted with ethyl acetate. The extract was washed with water and dried over anhydrous magnesium sulfate, and the solvent was distilled off at reduced pressure to give 112 mg of 2-[[3-(trifluoromethyl)phenyl]amino][1,2,4]triazolo[1,5-a]pyridin-8-ol (yield 84%) in the form of a crude product. Trifluoromethanesulfonic anhydride (0.069 mL, 0.411 mmol) was added while cooled on ice to a pyridine (1.0 mL) solution of 2-[[3-(trifluoromethyl)phenyl]amino][1,2,4]triazolo[1,5-a]pyridin-8-ol (110 mg, 0.374 mmol); and the mixture was stirred for 2 hours at room temperature. Water was poured into the reaction solution, and the mixture was neutralized with 1 N hydrochloric acid aqueous solution and was extracted with ethyl acetate. The extract was washed with water and dried over anhydrous magnesium sulfate, and the solvent was distilled off at reduced pressure. The residue was purified by silica gel column chromatography (ethyl acetate:hexane=1:9) to give 105 mg of the titled compound (yield 66%) in solid form.

¹H-NMR (DMSO-d₆) δ: 6.91-6.98 (1H, m), 7.21-7.32 (2H, m), 7.41-7.50 (2H, m), 7.62-7.70 (1H, m), 7.96-8.04 (1H, m), 8.50 (1H, d, J=6.8 Hz).

### Reference Example 121

### Ethyl 3-(2-[[3-(trifluoromethyl)phenyl]amino][1,2,4]triazolo[1,5-a]pyridin-8-yl)benzoate

2-[[3-(Trifluoromethyl)phenyl]amino][1,2,4]triazolo[1,5-a]pyridin-8-yl trifluoromethanesulfonate (590 mg, 1.38 mmol) obtained in Reference Example 120, [3-(ethoxycarbonyl)phenyl]boronic acid (295 mg, 1.52 mmol), and tetrakis(triphenylphosphine)palladium (0) (192 mg, 0.166 mmol) in 2 N sodium carbonate aqueous solution (2.1 mL)-1,2-dimethoxyethane (12 mL) mixture were stirred for 4 hours at 95° C. in a nitrogen atmosphere. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate:hexane=3:7) to give 291 mg of the titled compound (yield 49%) in solid form.

¹H-NMR (CDCl₃) δ: 1.41 (3H, t, J=7.2 Hz), 4.42 (2H, q, J=7.2 Hz), 7.00-7.07 (1H, m), 7.19-7.33 (2H, m), 7.42 (1H, t, J=7.9 Hz), 7.59 (1H, t, J=7.9 Hz), 7.63-7.72 (2H, m), 7.98-8.02 (1H, m), 8.06-8.11 (1H, m), 8.27-8.33 (1H, m), 8.49 (1H, dd, J=6.8, 1.1 Hz), 8.61 (1H, t, J=1.1 Hz).

### Reference Example 122

### 3-(2-[[3-(Trifluoromethyl)phenyl]amino][1,2,4]triazolo[1,5-a]pyridin-8-yl)benzoic acid

1 N sodium hydroxide aqueous solution (1.3 mL) was added at room temperature to a THF (4 mL)-methanol (2 mL) mixed solution of ethyl 3-(2-[[3-(trifluoromethyl)phenyl]amino][1,2,4]triazolo[1,5-a]pyridin-8-yl)benzoate (270 mg, 0.633 mmol) obtained in Reference Example 121, and the mixture was stirred over night. Water was poured into the reaction solution, the pH was adjusted to between 2 and 3 with 1 N hydrochloric acid aqueous solution, and the mixture was extracted with ethyl acetate. The extract was washed with water and dried over anhydrous magnesium sulfate, and the solvent was distilled off at reduced pressure. Diethyl ether was added to the residue, and 198 mg of the titled compound (yield 79%) was filtered off in solid form.

¹H-NMR (DMSO-d₆) δ: 7.11-7.29 (2H, m), 7.54 (1H, t, J=8.0 Hz), 7.67 (1H, t, J=8.0 Hz), 7.89-8.07 (3H, m), 8.18 (1H, s), 8.36 (1H, dd, J=6.6, 1.7 Hz), 8.70-8.76 (1H, m), 8.88 (1H, dd, J=6.6, 1.7 Hz), 10.19 (1H, s).

### Reference Example 123

### 4-Bromo-2-[3-(trifluoromethyl)benzyl]-2,3-dihydro-1H-isoindol-1-one

Sulfuric acid (1.0 mL) was added to a methanol (50 mL) solution of 3-bromo-2-methylbenzoic acid (6.70 g, 31.2 mmol), and the mixture was heated to reflux for 16 hours. The solvent was distilled off at reduced pressure, water was added to the residue, and the mixture was extracted with ethyl acetate. The organic layer was washed with saturated brine, then dried over anhydrous sodium sulfate, and then concentrated at reduced pressure to give 6.70 g of methyl 3-bromo-2-methylbenzoate crude product. N-bromosuccinimide (5.33 g, 30.0 mmol) and 2,2′-azobis(2-methylpropionitrile) (10 mg) were added to a chlorobenzene (50 mL) solution of this compound (6.70 g, 29.2 mmol), and the mixture was heated to reflux for 16 hours. The inorganic material was filtered off, and the filtrate was washed with saturated sodium bicarbonate aqueous solution, then dried over anhydrous sodium sulfate, and concentrated at reduced pressure to give 8.0 g of methyl 3-bromo-2-(bromomethyl)benzoate crude product. 1-[3-(trifluoromethyl)phenyl]methanamine (5.0 g, 28.5 mmol) and potassium carbonate (4.3 g, 31.2 mmol) were added to a toluene (50 mL) solution of this compound (8.0 g, 26.0 mmol), and the mixture was heated to reflux for 16 hours. The inorganic material was filtered off, and the filtrate was washed with saturated sodium bicarbonate aqueous solution, then dried over anhydrous sodium sulfate, and concentrated at reduced pressure. The residue was crystallized using ethyl acetate-hexane to give 1.72 g of the titled compound (yield 65%). Melting point: 119-120° C.

¹H-NMR (CDCl₃) δ: 4.21 (2H, s), 4.87 (2H, s), 7.39 (1H, t, J=7.5 Hz), 7.43-7.58 (4H, m), 7.67 (1H, d, J=7.8 Hz), 7.85 (1H, d, J=7.2 Hz).

### Reference Example 124

### 3-[1-Oxo-2-[3-(trifluoromethyl)benzyl]-2,3-dihydro-1H-isoindol-4-yl]benzoic acid

4-Bromo-2-[3-(trifluoromethyl)benzyl]-2,3-dihydro-1H-isoindol-1-one obtained in Reference Example 123 was used in the same manner as in Reference Example 10 to obtain the titled compound. Yield: 65%; melting point: 190-191° C. (ethyl acetate-hexane).

¹H-NMR (CDCl₃) δ: 4.37 (2H, s), 4.88 (2H, s), 7.19-7.71 (8H, m), 7.93-7.98 (1H, m), 8.10-8.17 (2H, m), 1H unconfirmed.

### Reference Example 125

### 3-Bromo-2-hydrazinopyridine

A THF solution (150 mL) of 3-bromo-2-chloropyridine (6.74 g, 35 mmol) and hydrazine monohydrate (8.49 mL, 0.75 mol) was stirred for 18 hours at 65° C. Hydrazine monohydrate (5.09 mL, 0.45 mol) was furthermore added, and the mixture was stirred for 24 hours. The reaction solution was concentrated at reduced pressure, diluted with water, and extracted with ethyl acetate. The extract was washed with water and dried over anhydrous magnesium sulfate, and the solvent was then distilled off at reduced pressure. The precipitated crystals were filtered off to give 2.36 g of the titled compound (yield 36%). Melting point: 146-148° C.

¹H-NMR (DMSO-d₆) δ: 4.22 (2H, s), 6.55 (1H, t, J=6.2 Hz), 7.36 (1H, br s), 7.73 (1H, d, J=7.8 Hz), 8.08 (1H, d, J=4.5 Hz).

### Reference Example 126

### 8-Bromo[1,2,4]triazolo[4,3-a]pyridin-3(2H)-one

Carbonyl diimidazole (0.65 g, 3.99 mmol) was added to a THF (10 mL) solution of 3-bromo-2-hydrazinopyridine (0.5 g, 2.66 mmol) obtained in Reference Example 125, and the mixture was stirred for 3 hours. The reaction solution was diluted with water and extracted with ethyl acetate. The extract was allowed to flow through a small amount of silica gel, and the solvent was distilled off at reduced pressure. The precipitated crystals were filtered off to give 0.48 g of the titled compound (yield 84%). Melting point: 297-299° C.

¹H-NMR (DMSO-d₆) δ: 6.50 (1H, t, J=6.6 Hz), 7.57 (1H, d, J=6.6 Hz), 7.87 (1H, d, J=6.6 Hz), 12.71 (1H, s).

### Reference Example 127

### 8-Bromo-2-[3-(trifluoromethyl)benzyl][1,2,4]triazolo[4,3-a]pyridin-3(2H)-one

3-Trifluoromethylbenzyl bromide (1.85 mL, 12.1 mmol) was added to an acetone (30 mL) mixture of 8-bromo[1,2,4]triazolo[4,3-a]pyridin-3(2H)-one (2.16 g, 10.1 mmol) obtained in Reference Example 126 and potassium carbonate (2.79 g, 20.2 mmol), and the mixture was stirred for 2 hours at 55° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane-ethyl acetate=2:1) to give 3.18 g of the titled compound (yield 85%) in the form of crystals. Melting point: 111-113° C.

¹H-NMR (CDCl₃) δ: 5.26 (2H, s), 6.42 (1H, t, J=6.9 Hz), 7.38 (1H, d, J=7.2 Hz), 7.46 (1H, t, J=7.6 Hz), 7.50-7.65 (2H, m), 7.68 (1H, s), 7.79 (1H, d, J=6.9 Hz).

### Reference Example 128

### Ethyl 3-[3-oxo-2-[3-(trifluoromethyl)benzyl]-2,3-dihydro[1,2,4]triazolo[4,3-a]pyridin-8-yl]benzoate

A mixture of 8-bromo-2-[3-(trifluoromethyl)benzyl][1,2,4]triazolo[4,3-a]pyridin-3(2H)-one (2.7 g, 7.26 mmol) obtained in Reference Example 127, [3-(ethoxycarbonyl)phenyl]boronic acid (1.69 g, 8.71 mmol), tetrakis(triphenylphosphine)palladium (0) (0.42 g, 0.36 mmol), sodium carbonate (1.54 g, 14.5 mmol), water (10 mL), and dimethoxyethane (30 mL) was stirred for 21 hours at 80° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane-ethyl acetate=2:1 to 1:1) to give 2.0 g of the titled compound (yield 62%) in the form of crystals. Melting point: 98-99° C.

¹H-NMR (CDCl₃) δ: 1.39 (3H, t, J=7.2 Hz), 4.40 (2H, q, J=7.2 Hz), 5.26 (2H, s), 6.64 (1H, t, J=6.6 Hz), 7.32 (1H, d, J=6.6 Hz), 7.40-7.60 (3H, m), 7.63 (1H, d, J=7.5 Hz), 7.71 (1H, s), 7.81 (1H, d, J=6.9 Hz), 8.05-8.20 (2H, m), 8.48 (1H, s).

### Reference Example 129

### 3-[3-Oxo-2-[3-(trifluoromethyl)benzyl]-2,3-dihydro[1,2,4]triazolo[4,3-a]pyridin-8-yl]benzoic acid

1 N sodium hydroxide aqueous solution (5.71 mL, 5.71 mmol) was added to an ethanol (15 mL) and THF (10 mL) solution of ethyl 3-[3-oxo-2-[3-(trifluoromethyl)benzyl]-2,3-dihydro[1,2,4]triazolo[4,3-a]pyridin-8-yl]benzoate (0.84 g, 1.90 mmol) obtained in Reference Example 128, and the mixture was stirred for 4 hours. The reaction solution was neutralized with the addition of 1 N hydrochloric acid (5.71 mL), diluted with water, and then extracted with ethyl acetate. The extract was washed with water and dried over anhydrous magnesium sulfate, and the solvent was then distilled off at reduced pressure. The precipitated crystals were filtered off to give 0.69 g of the titled compound (yield 88%). Melting point: 218-222° C.

¹H-NMR (DMSO-d₆) δ: 5.30 (2H, s), 6.78 (1H, t, J=6.9 Hz), 7.75 (1H, s), 7.97 (2H, t, J=7.5 Hz), 8.15 (1H, d, J=7.5 Hz), 8.49 (1H, s), 13.10 (1H, br s).

### Reference Example 130

### 2-Chloro-4-(3-nitrophenyl)pyrimidine

(3-Nitrophenyl)boronic acid (2.2 g, 13.2 mmol), tetrakis(triphenylphosphine)palladium (0) (0.77 g, 0.67 mmol), and 2 M sodium carbonate aqueous solution (8 mL) were added to a dimethoxyethane (100 mL) solution of 2,4-dichloropyrimidine (2.0 g, 13.4 mmol), and the mixture was heated to reflux for 13 hours in an argon atmosphere. Ethyl acetate was added to the reaction solution, and the solution was washed with water and with saturated brine, dried, and concentrated. The residue was purified by silica gel column chromatography (THF) and then recrystallized from ethyl acetate to give 1.2 g of the titled compound (yield 39%).

¹H-NMR (CDCl₃) δ: 7.72-7.77 (2H, m), 8.39-8.43 (1H, m), 8.47-8.51 (1H, m), 8.77 (1H, d, J=5.1 Hz), 8.93 (1H, t, J=2.1 Hz).

### Reference Example 131

### (4-(3-Nitrophenyl)pyrimidin-2-yl)-(2-(3,4-dimethoxyphenyl)ethyl)amine

2-(3,4-Dimethoxyphenyl)ethylamine (1.3 g, 7.2 mmol) and ethyl diisopropylamine (1.7 mL, 9.5 mmol) were added to an n-butanol (15 mL) solution of 2-chloro-4-(3-nitrophenyl)pyrimidine (1.1 g, 4.7 mmol) synthesized in Reference Example 130, and the mixture was heated for 30 min to 130° C. while irradiated with microwaves. Ethyl acetate was dissolved in the reaction solution, and the solution was washed with water and with saturated brine, dried, and concentrated. The residue was purified by silica gel column chromatography (ethyl acetate) and recrystallized from ethyl acetate-hexane to give 1.5 g of the titled compound (yield 84%).

¹H-NMR (CDCl₃) δ: 2.93 (2H, t, J=6.9 Hz), 3.75-3.82 (2H, m), 3.87 (3H, H, s), 3.88 (3H, s), 5.30 (1H, br t, J=5.7 Hz), 6.78 (1H, s), 6.84 (2H, d, J=0.6 Hz), 7.03 (1H, d, J=5.4 Hz), 7.65 (1H, t, J=8.1 Hz), 8.30-8.37 (2H, m), 8.41 (1H, d, J=5.1 Hz), 8.92 (1H, br s).

### Reference Example 132

### (4-(3-Aminophenyl)pyrimidin-2-yl)-(2-(3,4-dimethoxyphenyl)ethyl)amine

10%-palladium carbon (0.13 g) was added to a THF-ethanol (1:1, 40 mL) solution of (4-(3-nitrophenyl)pyrimidin-2-yl)-(2-(3,4-dimethoxyphenyl)ethyl)amine (1.3 g, 3.4 mmol) synthesized in Reference Example 131, and the mixture was stirred for 1 day at room temperature in a hydrogen atmosphere at ordinary pressure. The reaction solution was filtered and concentrated. The residue was purified by basic silica gel column chromatography (hexane-ethyl acetate=10:0 to 0:10) and was recrystallized from ethyl acetate-hexane to give 1.2 g (quantitative) of the titled compound.

¹H-NMR (CDCl₃) δ: 2.91 (2H, t, J=6.9 Hz), 3.72-3.82 (4H, m), 3.87 (6H, s), 5.21 (1H, br t, J=5.7 Hz), 6.78-6.84 (4H, m), 6.93 (1H, d, J=5.1 Hz), 7.22-7.27 (1H, m), 7.36-7.39 (2H, m), 8.31 (1H, d, J=5.1 Hz).

### Reference Example 133

### 2-Chloro-6-(2-(3,4-dimethoxyphenyl)ethoxy)pyridine

A THF (30 mL) solution of 6-chloropyridin-2-ol (1.0 g, 7.72 mmol), 2-(3,4-dimethoxyphenyl)ethanol (1.55 g, 8.44 mmol), triphenylphosphine (2.23 g, 8.44 mmol), and diethyl azodicarboxylate (1.61 g, 8.44 mmol) was stirred for 1 hour at room temperature. The reaction mixture was concentrated at reduced pressure, and the residue was chromatographed on a silica gel column (hexane-ethyl acetate 80:20) to give 1.76 g of the titled compound (yield 78%) in the form of an oily substance.

¹H-NMR (CDCl₃) δ: 3.01 (2H, t, J=6.9 Hz), 3.86 (3H, s), 3.88 (3H, s), 4.49 (2H, t, J=6.9 Hz), 6.60-6.89 (5H, m), 7.45-7.92 (1H, m).

### Reference Example 134

### N-(3-(2-(2-(3,4-dimethoxyphenyl)ethylamino)pyrimidin-4-yl)phenyl)-2-methoxyacetamide

Methoxyacetic acid (46 mg, 0.51 mmol), WSC (99 mg, 0.52 mmol), and HOBt (70 mg, 0.52 mmol) were added to a DMF (3 mL) solution of (4-(3-aminophenyl)pyrimidin-2-yl)-(2-(3,4-dimethoxyphenyl)ethyl)amine (0.15 g, 0.43 mmol) synthesized in Reference Example 132, and the mixture was stirred for 16 hours at room temperature. Ethyl acetate was added to the reaction solution, the solution was washed with water and with saturated brine, and the mixture was concentrated. The residue was purified by silica gel column chromatography (hexane-ethyl acetate=10:0 to 0:10) and was recrystallized from ethyl acetate-hexane to give 0.14 g (yield 77%) of the titled compound in the form of crystals. Melting point: 140-141° C.

¹H-NMR (CDCl₃) δ: 2.92 (2H, t, J=6.9 Hz), 3.53 (3H, s), 3.74-3.80 (2H, m), 3.87 (6H, s), 4.05 (2H,s), 5.22 (1H, br t, J=5.7 Hz), 6.78 (1H, s), 6.82 (2H, s), 6.98 (1H, d, J=5.1 Hz), 7.44 (1H, d, J=8.1 Hz), 7.76-7.80 (2H, m), 8.19 (1H, br s), 8.33-8.36 (2H, m).

### Reference Example 135

### 3-(6-(2-(3,4-dimethoxyphenyl)ethoxy)pyridin-2-yl)benzoic acid

2-Chloro-6-(2-(3,4-dimethoxyphenyl)ethoxy)pyridine obtained in Reference Example 133 and [3-(ethoxycarbonyl)phenyl]boronic acid were used in the same manner as in Reference Example 10 to obtain the titled compound. Yield: 36%; melting point: 147-148° C. (ethyl acetate-hexane).

¹H-NMR (CDCl₃) δ: 3.11 (2H, t, J=7.2 Hz), 3.86 (3H, s), 3.88 (3H, s), 4.66 (2H, t, J=7.2 Hz), 6.72 (1H, d, J=8.1 Hz), 6.82-6.91 (3H, m), 7.41 (1H, d, J=7.5 Hz), 7.57 (1H, t, J=7.5 Hz), 7.66 (1H, t, J=7.5 Hz), 8.14 (1H, dd, J=7.5, 1.2 Hz).8.30 (1H, d, J=6.6 Hz), 8.76 (1H, s), 1H unconfirmed.

### Reference Example 136

### 3-(6-(2-(3,4-dimethoxyphenyl)ethoxy)pyridin-2-yl)-N-(2-pyrrolidin-1-ylethyl)benzamide

3-(6-(2-(3,4-Dimethoxyphenyl)ethoxy)pyridin-2-yl)benzoic acid obtained in Reference Example 135 and 2-pyrrolidin-1-ylethanamine were used in the same manner as in Reference Example 134 to obtain the titled compound. Yield: 45%; melting point: 126-127° C. (ethyl acetate-hexane).

¹H-NMR (CDCl₃) δ: 1.73-1.82 (4H, m), 2.47-2.61 (4H, m), 2.70 (2H, t, J=6.0 Hz), 3.09 (2H, t, J=6.9 Hz), 3.57 (2H, q, J=6.0 Hz), 3.86 (3H, s), 3.87 (3H, s), 4.63 (2H, t, J=6.9 Hz), 6.70 (1H, d, J=7.8 Hz), 6.81-6.88 (4H, m), 7.38 (1H, d, J=7.2 Hz), 7.50 (1H, t, J=7.5 Hz), 7.64 (1H, t, J=8.1 Hz), 7.76 (1H, d, J=8.4 Hz), 8.16 (1H, d, J=8.4 Hz), 8.42 (1H, t, J=1.5 Hz).

### Reference Example 137

### 3-Chloro-N-methyl-2-nitroaniline

A mixture of 1,3-dichloro-2-nitrobenzene (4.07 g, 21.2 mmol), methylamine (40% aqueous solution, 2.31 mL, 29.7 mmol), and DBU (4.44 mL, 29.7 mmol) was heated to 100° C. for 18 hours. The reaction solution was diluted with water and extracted with ethyl acetate. The extract was washed with water and with saturated brine, dried, and concentrated. The residue was purified by silica gel column chromatography (hexane-methylene chloride 80:20→0:100) to give 10.1 g of the titled compound (yield 35%).

¹H-NMR (CDCl₃) δ: 2.93 (3H, d, J=4.8 Hz), 5.92 (1H, br s), 6.70 (1H, d, J=8.0 Hz), 6.76 (1H, d, J=8.4 Hz), 7.24-7.25 (1H, m).

### Reference Example 138

### Ethyl 3′-(methylamino)-2′-nitrophenyl-3-carboxylate

A mixture of 3-chloro-N-methyl-2-nitroaniline (10.0 g, 53.6 mmol) obtained in Reference Example 137, [3-(ethoxycarbonyl)phenyl]boronic acid (14.5 g, 75.0 mmol), and tetrakis(triphenylphosphine)palladium (0) (2.76 g, 2.68 mmol) in 2 N sodium carbonate aqueous solution (80 mL)-toluene (180 mL) was reacted for 20 hours at 100° C. in a nitrogen atmosphere. The addition of water to the reaction solution was followed by extraction with methylene chloride. The organic layer was washed with water, dried over anhydrous sodium sulfate, and then concentrated at reduced pressure. The residue was purified by silica gel column chromatography (hexane-ethyl acetate 90:10) to give 14.9 g of the titled compound (yield 93%) in the form of an oily substance.

¹H-NMR (CDCl₃) δ: 1.39 (3H, t, J=7.2 Hz), 2.99 (3H, d, J=5.2 Hz), 4.38 (2H, q, J=7.2 Hz), 6.49 (1H, d, J=4.4 Hz), 6.62 (1H, dd, J=7.6, 1.2 Hz), 6.84 (1H, dd, J=8.4, 1.2 Hz), 7.38-7.43 (1H, m), 7.45 (2H, dd, J=4.0, 1.6 Hz), 8.00 (1H, dd, J=2.4, 1.6 Hz), 8.03-8.05 (1H, m).

### Reference Example 139

### Ethyl 2′-amino-3′-(methylamino)biphenyl-3-carboxylate

10%-palladium carbon (0.15 g) was added to an ethanol (200 mL) solution of ethyl 3′-(methylamino)-2′-nitrophenyl-3-carboxylate (13.0 g, 43.3 mmol) synthesized in Reference Example 138, and the mixture was stirred for 15 hours at room temperature in a hydrogen atmosphere at ordinary pressure. The reaction solution was filtered and concentrated to give 12.8 g of the titled compound (yield 98%).

¹H-NMR (CDCl₃) δ: 1.39 (3H, t, J=7.2 Hz), 2.92 (3H, s), 3.43 (3H, br s), 4.39 (2H, q, J=7.2 Hz), 5.69-6.73 (2H, m), 6.92 (1H, t, J=7.6 Hz), 7.53 (1H, t, J=7.6 Hz), 7.62-7.65 (1H, m), 8.04 (1H, dd, J=8.0, 1.2 Hz), 8.12-8.13 (1H, m).

### Reference Example 140

### Ethyl 3-(1-methyl-2-oxo-2,3-dihydro-1H-benzimidazol-4-yl)benzoate

A tetrahydrofuran (500 mL) solution of ethyl 2′-amino-3′-(methylamino)biphenyl-3-carboxylate (12.8 g, 47.4 mmol) synthesized in Reference Example 139 and N,N′-carbonyl diimidazole (20 g, 120 mmol) was stirred for 18 hours at 50° C. The reaction solution was diluted with ethyl acetate and washed with water. The solution was dried over anhydrous sodium sulfate, and the solvent was then distilled off at reduced pressure to give 10.4 g of the titled compound (yield 98%) in the form of crystals.

¹H-NMR (CDCl₃) δ: 1.42 (3H, t, J=7.2 Hz), 3.45 (3H, s), 4.42 (2H, q, J=7.2 Hz), 7.01 (1H, d, J=7.6 Hz), 7.16 (1H, dd, J=8.0, 1.2 Hz), 7.21 (1H, t, J=8.0 Hz), 7.59 (1H, t, J=8.0 Hz), 7.71 (1H, ddd, J=7.6, 2.2, 1.2 Hz), 7.90 (1H, br s), 8.09 (1H, ddd, J=8.0, 1.4, 1.2 Hz), 8.18-8.19 (1H, m).

### Reference Example 141

### Ethyl 3-(2-chloro-1-methyl-1H-benzimidazol-4-yl)benzoate

A mixture of ethyl 3-(1-methyl-2-oxo-2,3-dihydro-1H-benzimidazol-4-yl)benzoate (10.0 g, 33.7 mmol) synthesized in Reference Example 140 and phosphoryl chloride (19.5 mL, 674 mmol) was stirred for 4 hours at 100° C. Water was slowly added while cooled on ice, and the solution was neutralized with potassium carbonate. The solution was extracted with methylene chloride, washed with water, and dried over anhydrous sodium sulfate, and the solvent was then distilled off at reduced pressure to give 10.3 g of the titled compound (yield 97%).

¹H-NMR (CDCl₃) δ: 1.41 (3H, t, J=7.2 Hz), 3.84 (3H, s), 4.41 (2H, q, J=7.2 Hz), 7.31 (1H, dd, J=8.0, 1.2 Hz), 7.40 (1H, t, J=8.0 Hz), 7.48 (1H, dd, J=7.6, 1.2 Hz), 7.57 (1H, t, J=8.0 Hz), 8.05 (1H, ddd, J=8.0, 1.4, 1.4 Hz), 8.25 (1H, ddd, J=7.6, 2.0, 1.2 Hz), 8.52 (1H, dd, J=1.2, 1.2 Hz).

### Reference Example 142

### Ethyl 3-[1-methyl-2-[[3-(trifluoromethyl)phenyl]amino]-1H-benzimidazol-4-yl]benzoate

3-(Trifluoromethyl)aniline (1.29 mL, 10.3 mmol) was added to a mixture of ethyl 3-(2-chloro-1-methyl-1H-benzimidazol-4-yl)benzoate (2.50 g, 7.94 mmol) obtained in Reference Example 141, cesium carbonate (7.76 g, 23.8 mmol), Tris(dibenzylideneacetone)dipalladium (0) (218 mg, 0.238 mmol), and 2-(dicyclohexylphosphino)-2′,4′,6′-triisopropyl-1,1 ′-biphenyl (454 mg, 0.953 mmol) in toluene (40 mL), and the mixture was stirred for 8 hours at 85° C. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate:hexane=1:2) to give 1.35 g of the titled compound (yield 39%) in solid form.

¹H-NMR (CDCl₃) δ: 1.38 (3H, t, J=7.2 Hz), 3.49 (3H, s), 4.39 (2H, q, J=7.2 Hz), 7.18 (5H, m), 7.42 (1H, dd, J=7.6, 1.2 Hz), 7.47 (1H, dd, J=8.4, 2.0 Hz), 7.55 (1H, t, J=8.0), 7.91 (1H, br s), 8.03 (1H, ddd, J=8.0, 1.4, 1.2 Hz), 8.37 (1H, d, J=7.6 Hz), 8.72 (1H, br s).

### Reference Example 143

### Ethyl 3-[1-methyl-2-[3-(trifluoromethyl)phenoxy]-1H-benzimidazol-4-yl]benzoate

3-(Trifluoromethyl)phenol (1.16 mL, 9.53 mmol) was added to a mixture of ethyl 3-(2-chloro-1-methyl-1H-benzimidazol-4-yl)benzoate (2.50 g, 7.94 mmol) obtained in Reference Example 141 and cesium carbonate (7.76 g, 23.8 mmol) in DMF (15 mL), and the mixture was stirred for 15 hours at 85° C. Water was poured into the reaction solution, and the mixture was extracted with ethyl acetate. The extract was washed with 1 N sodium hydroxide aqueous solution and dried over anhydrous magnesium sulfate, and the solvent was distilled off at reduced pressure. The residue was purified by silica gel column chromatography (ethyl acetate:hexane=4:1) to give 3.46 g of the titled compound (yield 99%) in the form of an oily substance.

¹H-NMR (CDCl₃) δ: 1.37 (3H, t, J=7.2 Hz), 3.79 (3H, s), 4.38 (2H, q, J=7.2 Hz), 7.26-7.29 (1H, m), 7.35 (1H, t, J=7.6 Hz), 7.46-7.50 (3H, m), 7.54 (1H, t, J=8.0 Hz), 7.77-7.80 (1H, m), 7.88-7.89 (1H, m), 7.98-8.00 (1H, m), 8.25 (1H, ddd, J=7.8, 1.6, 1.2 Hz), 8.59 (1H, dd, J=1.6, 1.2 Hz).

### Reference Example 144

### Ethyl 3-[1-methyl-2-[[3-(trifluoromethyl)phenyl]sulfanyl]-1H-benzimidazol-4yl]benzoate

Ethyl 3-(2-chloro-1-methyl-1H-benzimidazol-4-yl)benzoate obtained in Reference Example 141 and 3-(trifluoromethyl)benzenethiol were used in the same manner as in Reference Example 143 to obtain the titled compound. Yield: 97%.

¹H-NMR (DMSO-d₆) δ: 1.40 (3H, t, J=7.2 Hz), 3.81 (3H, s), 4.40 (2H, q, J=7.2 Hz), 7.34 (1H, dd, J=8.0, 1.2 Hz), 7.41-7.46 (2H, m), 7.51-7.54 (3H, m), 7.5-7.59 (1H, m), 7.81-7.82 (1H, m), 8.04 (1H, ddd, J=7.6, 1.6, 1.2 Hz), 8.33 (1H, ddd, J=7.6, 2.0, 1.2 Hz), 8.57-8.58 (1H, m).

### Reference Example 145

### (7-Bromo-1-benzofuran-2-yl)(3-chlorophenyl)methanone

3-Bromo-2-hydroxybenzaldehyde and 2-bromo-1-(3-chlorophenyl)ethanone were used in the same manner as in Reference Example 8 to obtain the titled compound. Yield: 22%.

¹H-NMR (CDCl₃) δ: 7.24 (1H, t, J=7.8 Hz), 7.51 (1H, t, J=7.8 Hz), 7.61-7.64 (1H, m), 7.66 (1H,s), 7.67-7.71 (2H, m), 8.05 (1H, dt, J=7.6, 1.2 Hz), 8.14 (1H, t, J=2.0 Hz)

### Reference Example 146

### 7-Bromo-2-(3-chlorobenzyl)-1-benzofuran

(7-Bromo-1-benzofuran-2-yl)(3-chlorophenyl)methanone obtained in Reference Example 145 was used in the same manner as in Reference Example 9 to obtain the titled compound. Yield: 78%.

¹H-NMR (CDCl₃) δ: 4.12 (2H, s), 6.40 (1H, s), 7.06 (1H, t, J=7.8 Hz), 7.19-7.21 (1H, m), 7.23-7.73 (2H, m), 7.31 (1H, s), 7.39 (2H, t, J=7.6 Hz).

### Reference Example 147

### Ethyl 3-[2-(3-chlorobenzyl)-1-benzofuran-7-yl]benzoate

7-Bromo-2-(3-chlorobenzyl)-1-benzofuran obtained in Reference Example 146 and [3-(ethoxycarbonyl)phenyl]boronic acid were used in the same manner as in Reference Example 4 to obtain the titled compound. Yield: 69%.

¹H-NMR (CDCl₃)67 : 1.41 (3H, t, J=7.2 Hz), 4.12 (2H, s), 4.42 (2H, q, J=14.2, 7.0 Hz), 6.45 (1H, s), 7.22-7.34 (5H, m), 7.44 (1H, d, J=7.6 Hz), 7.49 (1H, d, J=7.6 Hz), 7.56 (1H, t, J=7.8 Hz), 8.03-8.08 (2H, m), 8.52 (1H, s).

### Reference Example 148

### N-(2,6-dibromophenyl)-2-[3-(trifluoromethyl)phenyl]acetamide

DMF (0.1 mL) was added to a THF (20 mL) solution of [3-(trifluoromethyl)phenyl]acetic acid (3.67 g, 18.0 mmol), and oxalyl chloride (1.74 mL, 20 mmol) was then added dropwise while cooled on ice. The reaction solution was warmed to room temperature, stirred for 1 hour, and then concentrated at reduced pressure. The residue was dissolved in THF (30 mL). 2,6-dibromoaniline (3.0 g, 12.0 mmol), N-ethyl diisopropylamine (2.5 mL, 14.4 mmol), and 4-dimethylaminopyridine (1.75 g, 14.4 mmol) were added to the solution, and the mixture was stirred for 16 hours at room temperature. The addition of saturated sodium bicarbonate aqueous solution to the reaction solution was followed by extraction with ethyl acetate. The organic layer was washed with water and 1 N hydrochloric acid, and then dried over anhydrous sodium sulfate. The solvent was distilled off at reduced pressure, and the resulting residue was crystallized from hexane to give 2.94 g of the titled compound (yield 56%). Melting point: 169-170° C. (hexane)

¹H-NMR (CDCl₃) δ: 3.86 (2H, s), 6.88 (1H, br s), 7.01 (1H, t, J=8.1 Hz), 7.47-7.71 (6H, m).

### Reference Example 149

### N-(2,6-dibromophenyl)-2-[3-(trifluoromethyl)phenyl]ethanethioamide

Lawesson's reagent (2.95 g, 6.75 mmol) was added to a toluene (50 mL) solution of the N-(2,6-dibromophenyl)-2-[3-(trifluoromethyl)phenyl]acetamide (2.95 g, 6.75 mmol) obtained in Reference Example 148, and the mixture was heated to reflux for 1 hour. The insoluble material was filtered off, and the filtrate was concentrated at reduced pressure. The residue was purified by silica gel column chromatography (ethyl acetate-hexane 1:2) to give 2.9 g of the titled compound (yield 95%). Oily substance.

¹H-NMR (CDCl₃) δ: 4.35 (2H, s), 7.09 (1H, t, J=8.1 Hz), 7.50-7.71 (5H, m), 7.74 (1H, s), 8.09 (1H, br s).

### Reference Example 150

### 4-Bromo-2-[3-(trifluoromethyl)benzyl]-1,3-benzothiazole

A mixture of the N-(2,6-dibromophenyl)-2-[3-(trifluoromethyl)phenyl]ethanethioamide (2.90 g, 6.4 mmol) obtained in Reference Example 149, cesium carbonate (7.76 g, 23.8 mmol), Tris(dibenzylideneacetone)dipalladium (0) (292 mg, 0.32 mmol), and 2-(di-tert-butylphosphino)-1,1′-biphenyl (114 mg, 0.38 mmol) in 1,4-dioxane (40 mL) was stirred for 18 hours at 80° C. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate:hexane=10:90→50:50) to give 1.3 g of the titled compound (yield 55%) in the form of an oily substance.

¹H-NMR (CDCl₃) δ: 4.55 (2H, s), 7.21 (1H, t, J=9.8 Hz), 7.41-7.75 (6H, m).

### Reference Example 151

### 3-Bromo-2,6-difluorobenzaldehyde

Lithium diisopropylamide (2 M THF solution, 13 mL, 26 mmol) was added at −78° C. to a THF (80 mL) solution of 1-bromo-2,4-difluorobenzene (5.0 g, 25.9 mmol), and the mixture was stirred for 1 hour at the same temperature. DMF (2.0 g, 28 mmol) was added to the solution, and the mixture was stirred for another 30 min. The addition of water to the reaction solution was followed by extraction with ethyl acetate. The combined organic layers were washed with 1 N hydrochloric acid and water, then dried over anhydrous magnesium sulfate, and then concentrated at reduced pressure, and the residue was purified by silica gel column chromatography (ethyl acetate-hexane 1:9) to give 3.0 g of the titled compound (yield 32%). Melting point: 53-54° C.

¹H-NMR (CDCl₃) δ: 7.00 (1H, dt, J=9.3, 1.8 Hz), 7.71-7.82 (1H, m), 10.3 (1H, s).

### Reference Example 152

### 3-Bromo-6-chloro-2-fluorobenzaldehyde

1-Bromo-4-chloro-2-fluorobenzene was used in the same manner as in Reference Example 151 to obtain the titled compound. Yield: 45%.

¹H-NMR (CDCl₃) δ: 7.19 (1H, dd, J=8.4, 1.5 Hz), 7.64-7.87 (1H, m), 10.4 (1H, s).

### Reference Example 153

### Ethyl 7-bromo-3-methyl-1-benzothiophene-2-carboxylate

Ethyl thioglycolate (4.82 mL, 43.8 mmol) was added to a DMSO (70 mL) suspension of sodium hydride (2.77 g, 69.2 mmol), and the mixture was stirred for 15 min. A DMSO (30 mL) solution of 1-(3-bromo-2-fluorophenyl)ethanone (10.0 g, 46.1 mmol) was added dropwise to the reaction solution, and the mixture was then stirred for 2 hours at room temperature in a nitrogen atmosphere. The reaction solution was poured into ice, and the mixture was extracted with ethyl acetate. The combined organic layers were washed with water, dried over anhydrous magnesium sulfate, and then concentrated at reduced pressure. The residue was purified by silica gel column chromatography (hexane-ethyl acetate 100:0→90:10) to give 10.5 g of the titled compound (yield 76%) in the form of crystals. Melting point: 67-70° C. (ethyl acetate).

¹H-NMR (CDCl₃) δ: 1.43 (3H, t, J=7.2 Hz), 2.75 (3H, s), 4.41 (2H, q, J=7.2 Hz), 7.27-7.35 (1H, m), 7.61 (1H, dd, J=7.5, 0.9 Hz), 7.79 (1H, dd, J=8.1, 0.9 Hz).

### Reference Example 154

### Ethyl 7-bromo-4-fluoro-1-benzothiophene-2-carboxylate

An acetonitrile (20 mL)-DMSO solution (5 mL) of 3-bromo-2,6-difluorobenzaldehyde (1.30 g, 5.88 mmol) synthesized in Reference Example 151 was added dropwise at 0° C. to an acetonitrile (10 mL) solution of ethyl thioglycolate (707 mg, 5.88 mmol) and triethylamine (1.22 mL, 8.8 mmol), the mixture was stirred for 1 hour at 0° C., and the solution was stirred for 16 hours while gradually warmed to room temperature. Water and 6 N hydrochloric acid were added to the reaction solution, and the mixture was stirred while cooled on ice. The precipitated crystals were filtered off and recrystallized from ethanol to give 1.1 g of the titled compound (yield 62%). Melting point: 73-74° C.

¹H-NMR (CDCl₃) δ: 1.43 (3H, t, J=6.9 Hz), 4.42 (2H, q, J=6.9 Hz), 6.98 (1H, t, J=8.7 Hz), 7.51-7.56 (1H, m), 8.21 (1H, s).

### Reference Example 155

### Ethyl 7-chloro-4-fluoro-1-benzothiophene-2-carboxylate

3-Chloro-2,6-difluorobenzaldehyde was used in the same manner as in Reference Example 154 to obtain the titled compound. Yield: 92%; melting point: 63-64° C. (ethyl acetate-hexane).

¹H-NMR (CDCl₃) δ: 1.43 (3H, t, J=6.9 Hz), 4.42 (2H, q, J=6.9 Hz), 7.03 (1H, t, J=9.0 Hz), 7.34-7.40 (1H, m), 8.13 (1H, s).

### Reference Example 156

### Ethyl 7-bromo-4-chloro-1-benzothiophene-2-carboxylate

An acetonitrile (10 mL) solution of 3-bromo-6-chloro-2-fluorobenzaldehyde (5.50 g, 24.8 mmol) synthesized in Reference Example 152 was added dropwise at 0° C. to an acetonitrile (40 mL) solution of ethyl thioglycolate (2.80 g, 23.6 mmol) and triethylamine (4.9 mL, 35.4 mmol), and the mixture was stirred for 3 hours at 60° C. The addition of water to the reaction solution was followed by extraction with ethyl acetate, and the organic layer was washed with water and 1 N hydrochloric acid, dried over anhydrous magnesium sulfate, and then concentrated at reduced pressure. The resulting residue was purified by silica gel column chromatography (hexane-ethyl acetate 95:5→70:30) to give 5.36 g of the titled compound (yield 71%).

¹H-NMR (DMSO-d₆) δ: 1.43 (3H, t, J=6.9 Hz), 4.43 (2H, q, J=6.9 Hz), 7.28 (1H, d, J=8.1 Hz), 7.50 (1H, d, J=8.1 Hz), 8.24 (1H, s).

### Reference Example 157

### 7-Bromo-3-methyl-1-benzothiophene-2-carboxylic acid

Ethyl 7-bromo-3-methyl-1-benzothiophene-2-carboxylate obtained in Reference Example 153 was used in the same manner as in Reference Example 5 to obtain the titled compound. Yield: 80%; melting point: 311-312° C. (ethyl acetate).

¹H-NMR (DMSO-d₆) δ: 2.71 (3H, s), 7.46 (1H, t, J=8.0 Hz), 7.79 (1H, d, J=6.8 Hz), 8.01 (1H, d, J=7.2 Hz), 13.61 (1H, br s).

### Reference Example 158

### 7-Bromo-4-fluoro-1-benzothiophene-2-carboxylic acid

2 N sodium hydroxide aqueous solution (5.0 mL, 10.0 mmol) was added to a THF (40 mL)-methanol (10 mL) mixed solution of ethyl 7-bromo-4-fluoro-1-benzothiophene-2-carboxylate (2.00 g, 6.60 mmol) synthesized in Reference Example 154, and the mixture was stirred for 1 hour at room temperature. The reaction solution was neutralized with the addition of 1 N hydrochloric acid, diluted with water, and then extracted with ethyl acetate. The organic layer was washed with water, dried over anhydrous magnesium sulfate, and then concentrated at reduced pressure to give 1.40 g of the titled compound (yield 77%) in the form of crystals. Melting point: 249-252° C.

¹H-NMR (DMSO-d₆) δ: 7.32 (1H, dd, J=9.9, 8.7 Hz), 7.80 (1H, dd, J=9.9, 4.5 Hz), 8.18 (1H, s), 1H unconfirmed.

### Reference Example 159

### 7-Chloro-4-fluoro-1-benzothiophene-2-carboxylic acid

Ethyl 7-chloro-4-fluoro-1-benzothiophene-2-carboxylate synthesized in Reference Example 155 was used in the same manner as in Reference Example 158 to obtain the titled compound. Yield: 90%, melting point: 248-249° C.

¹H-NMR (CDCl₃) δ: 7.07 (1H, t, J=8.7 Hz), 7.43 (1H, dd, J=8.4, 4.2 Hz), 8.26 (1H, s), 1H unconfirmed.

### Reference Example 160

### 7-Bromo-4-chloro-1-benzothiophene-2-carboxylic acid

Ethyl 7-bromo-4-chloro-1-benzothiophene-2-carboxylate synthesized in Reference Example 156 was used in the same manner as in Reference Example 158 to obtain the titled compound. Yield: 55%. Melting point: ≧300° C.

¹H-NMR (DMSO-d₆) δ: 7.54 (1H, d, J=8.4 Hz), 7.78 (1H, d, J=7.8 Hz), 8.10 (1H, s), 1H unconfirmed.

### Reference Example 161

### (4-Bromo-1-benzothiophen-2-yl)methanol

A 1.0 M borane-THF solution (6.86 mL, 8.10 mmol) was added a little at a time while cooled on ice to a THF solution (37 mL) of 4-bromo-1-benzothiophene-2-carboxylic acid (950 mg, 3.70 mmol), and the mixture was then stirred for 3 hours at 60° C. 1 N hydrochloric acid was added to the reaction solution to decompose the excess borane, and the mixture was then extracted with ethyl acetate. The extract was washed with water and dried over anhydrous sodium sulfate, and the solvent was then distilled off at reduced pressure. The residue was purified by silica gel column chromatography (hexane:ethyl acetate=90:10→75:25) to give 612 mg of the titled compound (yield 68%).

¹H-NMR (CDCl₃) δ: 1.91-2.00 (1H, m), 4.95 (2H, dd, J=6.0, 0.8 Hz), 7.13-7.19 (1H, m), 7.36-7.38 (1H, m), 7.50 (1H, dd, J=7.4, 0.8 Hz), 7.74 (1H, d, J=8.0 Hz).

### Reference Example 162

### (7-Bromo-1-benzothiophen-2-yl)methanol

While cooled on ice, 7-bromo-1-benzothiophene-2-carboxylic acid (0.52 g, 2.02 mmol) was added a little at a time to a 1.18 M borane-THF solution (6.86 mL, 8.10 mmol), and the mixture was then stirred for 3 hours at 60° C. The reaction solution was cooled on ice, 1 N hydrochloric acid was added to decompose the excess borane, and the mixture was then diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=4:1) and recrystallized from hexane-ethyl acetate to give 0.41 g of the titled compound (yield 83%). Melting point: 82-83° C.

¹H NMR (CDCl₃) δ: 1.94 (1H, t, J=6.0 Hz), 4.94 (2H, d, J=6.0 Hz), 7.21 (1H, d, J=7.8 Hz), 7.31 (1H, s), 7.47 (1H, d, J=7.8 Hz), 7.67 (1H, d, J=7.8 Hz).

### Reference Example 163

### (7-Bromo-3-methyl-1-benzothiophen-2-yl)methanol

A THF solution (0.9 mol/L, 119 mL, 107 mmol) of a borane-THF complex was added dropwise while cooled on ice to a THF (250 mL) solution of the 7-bromo-3-methyl-1-benzothiophene-2-carboxylic acid (7.26 g, 26.8 mmol) obtained in Reference Example 157, and the mixture was then stirred for 1 hour at 60° C. Drops of water were added while cooled on ice to the reaction solution, and the mixture was concentrated at reduced pressure and then extracted with ethyl acetate. The combined organic layers were dried over anhydrous magnesium sulfate, and then concentrated at reduced pressure. The residue was purified by silica gel column chromatography (hexane-ethyl acetate 90:10→60:40) to give 6.66 g of the titled compound (yield 97%). Melting point: 129-130° C. (ethyl acetate).

¹H-NMR (CDCl₃) δ: 1.86 (1H, t, J=5.9 Hz), 2.37 (3H, s), 4.93 (2H, d, J=6.1 Hz), 7.22-7.31 (1H, m), 7.48 (1H, d, J=7.6 Hz), 7.62 (1H, d, J=8.0 Hz).

### Reference Example 164

### (7-Bromo-4-fluoro-1-benzothiophen-2-yl)methanol

A THF solution (1 M; 16.2 mL, 16.2 mmol) of a borane-THF complex was added to a THF solution of the 7-bromo-4-fluoro-1-benzothiophene-2-carboxylic acid (1.40 g, 5.09 mmol) synthesized in Reference Example 158, and the mixture was stirred for 2 hours at 60° C. While cooled on ice, water was added to the reaction solution until no foam was formed, and the mixture was stirred for 30 min at room temperature. The mixture was extracted with ethyl acetate, and the organic layer was washed with water and 1 N hydrochloric acid, dried over anhydrous magnesium sulfate, and then concentrated at reduced pressure. The resulting residue was purified by silica gel column chromatography (hexane-ethyl acetate 5:95→50:50) to give 800 mg of the titled compound (yield 60%) in the form of crystals. Melting point: 80-81° C.

¹H-NMR (DMSO-d₆) δ: 2.01 (1H, t, J=6.0 Hz), 4.95 (2H, dd, J=6.0, 0.9 Hz), 6.92 (1H, dd, J=9.6, 8.1 Hz), 7.34-7.42 (2H, m).

### Reference Example 165

### (7-Chloro-4-fluoro-1-benzothiophen-2-yl)methanol

7-Chloro-4-fluoro-1-benzothiophene-2-carboxylic acid synthesized in Reference Example 159 was used in the same manner as in Reference Example 164 to obtain the titled compound. Yield: 84%. Melting point: 63-64° C.

¹H-NMR (CDCl₃) δ: 2.11 (1H, br s), 4.95 (2H, s), 6.97 (1H, dd, J=9.6, 8.1 Hz), 7.20-7.25 (1H, m), 7.33 (1H, s).

### Reference Example 166

### (7-Bromo-4-chloro-1-benzothiophen-2-yl)methanol

7-Bromo-4-chloro-1-benzothiophene-2-carboxylic acid synthesized in Reference Example 160 was used in the same manner as in Reference Example 164 to obtain the titled compound. Yield: 94%. Melting point: 141-143° C.

¹H-NMR (CDCl₃) δ: 2.04 (1H, br s), 4.95 (2H, s), 6.97 (1H, dd, J=9.3, 8.1 Hz), 7.20-7.25 (1H, m), 7.33 (1H, s).

### Reference Example 167

### Ethyl 3-[2-(hydroxymethyl)-1-benzothiophen-4-yl]benzoate

A 2 M sodium carbonate aqueous solution (2.8 mL)-1,2-dimethoxyethane (15 mL) mixed solution of (4-bromo-1-benzothiophen-2-yl)methanol (341 mg, 1.40 mmol) obtained in Reference Example 161, [3-(ethoxycarbonyl)phenyl]boronic acid (327 mg, 1.68 mmol), and tetrakis(triphenylphosphine)palladium (0) (65 mg, 0.056 mmol) was heated to reflux for 3 hours in a nitrogen atmosphere. The reaction solution was diluted with saturated brine and ethyl acetate, and was filtered using celite. The resulting filtrate was extracted with ethyl acetate. The combined organic layers were dried over anhydrous sodium sulfate, and then concentrated at reduced pressure, and the residue was purified by silica gel column chromatography (hexane-ethyl acetate 85:15→67:33) to give 399 mg of the titled compound (yield 91%).

¹H-NMR (CDCl₃) δ: 1.39 (3H, t, J=7.1 Hz), 2.13 (1H, br s), 4.39 (2H, q, J=7.1 Hz), 4.89 (2H, br s), 7.25 (1H, d, J=3.3 Hz), 7.30-7.43 (2H, m), 7.53 (1H, t, J=7.7 Hz), 7.68-7.75 (1H, m), 7.82 (1H, d, J=7.7 Hz), 8.04-8.09 (1H, m), 8.21 (1H, t, J=1.6 Hz).

### Reference Example 168

### Ethyl 3-[2-(hydroxymethyl)-1-benzothiophen-7-yl]benzoate

A mixture of (7-bromo-1-benzothiophen-2-yl)methanol (1.8 g, 7.40 mmol) obtained in Reference Example 162, [3-(ethoxycarbonyl)phenyl]boronic acid (1.72 g, 8.88 mmol), tetrakis(triphenylphosphine)palladium (0) (0.26 g, 0.22 mmol), sodium carbonate (1.57 g, 14.8 mmol), water (10 mL), and dimethoxyethane (30 mL) was stirred for 15 hours at 80° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=3:1) to give 2.20 g of the titled compound (yield 95%) in the form of an oily substance.

¹H NMR (CDCl₃) δ: 1.41 (3H, t, J=7.2 Hz), 1.93 (1H, t, J=6.0 Hz), 4.41 (2H, q, J=7.2 Hz), 4.93 (2H, d, J=6.0 Hz), 7.30 (1H, s), 7.36 (1H, d, J=6.9 Hz), 7.45 (1H, t, J=7.5 Hz), 7.56 (1H, t, J=7.8 Hz), 7.74 (1H, d, J=7.8 Hz), 7.91 (1H, d, J=7.8 Hz), 8.08 (1H, d, J=7.8 Hz), 8.36 (1H, s).

### Reference Example 169

### Ethyl 3-[2-(hydroxymethyl)-3-methyl-1-benzothiophen-7-yl]benzoate

(7-Bromo-3-methyl-1-benzothiophen-2-yl)methanol obtained in Reference Example 163 was used in the same manner as in Reference Example 4 to obtain the titled compound. 95% yield, oily substance.

¹H-NMR (CDCl₃) δ: 1.40 (3H, t, J=7.1 Hz), 1.94 (1H, t, J=5.7 Hz), 2.42 (3H, s), 4.40 (2H, q, J=7.2 Hz), 4.91 (2H, d, J=5.5 Hz), 7.36-7.42 (1H, m), 7.49 (1H, t, J=7.6 Hz), 7.55 (1H, t, J=7.8 Hz), 7.68 (1H, dd, J=8.0, 1.2 Hz), 7.89-7.94 (1H, m), 8.05-8.11 (1H, m), 8.36 (1H, t, J=1.6 Hz).

### Reference Example 170

### Ethyl 3-[4-fluoro-2-(hydroxymethyl)-1-benzothiophen-7-yl]benzoate

A mixture of (7-bromo-4-fluoro-1-benzothiophen-2-yl)methanol (800 mg, 30.6 mmol) synthesized in Reference Example 164, [3-(ethoxycarbonyl)phenyl]boronic acid (713 mg, 36.8 mmol), and tetrakis(triphenylphosphine)palladium (0) (177 mg, 0.153 mmol) in 2 N sodium carbonate aqueous solution (30 mL)-1,2-dimethoxyethane (30 mL) was heated to reflux for 9 hours. The reaction solution was diluted with saturated brine and ethyl acetate, and was filtered using celite. The resulting filtrate was extracted with ethyl acetate. The combined organic layers were dried over anhydrous sodium sulfate and then concentrated at reduced pressure, and the residue was purified by silica gel column chromatography (hexane-ethyl acetate 100:0→40:60) to give 810 mg of the titled compound (yield 80%). Melting point: 89-90° C.

¹H-NMR (CDCl₃) δ: 1.41 (3H, t, J=7.1 Hz), 1.95 (1H, t, J=6.0 Hz), 4.41 (2H, q, J=7.1 Hz), 4.94 (2H, dd, J=6.2, 1.0 Hz), 7.12 (1H, dd, J=9.6, 8.2 Hz), 7.31 (1H, dd, J=8.1, 4.8 Hz), 7.40-7.42 (1H, m), 7.56 (1H, t, J=7.7 Hz), 7.86 (1H, dq, J=7.7, 1.0 Hz), 8.09 (1H, dt, J=7.9, 1.4 Hz), 8.31 (1H, t, J=1.8 Hz).

### Reference Example 171

### Ethyl 3-[4-chloro-2-(hydroxymethyl)-1-benzothiophen-7-yl]benzoate

(7-Bromo-4-chloro-1-benzothiophen-2-yl)methanol synthesized in Reference Example 166 was used in the same manner as in Reference Example 4 to obtain the titled compound. Yield: 82%. Melting point: 119-120° C.

¹H-NMR (CDCl₃) δ: 1.41 (3H, t, J=6.9 Hz), 1.95 (1H, t, J=6.0 Hz), 4.41 (2H, q, J=6.9 Hz), 4.95 (2H, d, J=6.0 Hz), 7.28 (1H, d, J=7.8 Hz), 7.42-7.48 (2H, m), 7.56 (1H, t, J=7.8 Hz), 7.86 (1H, d, J=7.8 Hz), 8.09 (1H, d, J=8.1 Hz), 8.30 (1H, t, J=1.5 Hz).

### Reference Example 172

### [7-(4,4,5,5-tetramethyl-1,3,2-dioxaborolan-2-yl)-1-benzothiophen-2-yl]methanol

(7-Bromo-1-benzothiophen-2-yl)methanol obtained in Reference Example 162 was used in the same manner as in Reference Example 19 to obtain the titled compound. 87% yield, crude oily substance. The product was used, without further purification, in the following reaction.

¹H-NMR (CDCl₃) δ: 1.35-1.46 (12H, m), 4.93 (2H, s), 7.21 (1H, s), 7.35 (1H, t, J=7.5 Hz), 7.80 (2H, t, J=6.5 Hz).

### Reference Example 173

### [3-Methyl-7-(4,4,5,5-tetramethyl-1,3,2-dioxaborolan-2-yl)-1-benzothiophen-2-yl]methanol

(7-Bromo-3-methyl-1-benzothiophen-2-yl)methanol obtained in Reference Example 163 was used in the same manner as in Reference Example 19 to obtain the titled compound. 93% yield, crude oily substance. The product was used, without further purification, in the following reaction.

¹H-NMR (CDCl₃) δ: 1.35-1.45 (12H, m), 2.35-2.43 (3H, m), 4.92 (2H, s), 7.34-7.43 (1H, m), 7.79 (2H, dd, J=13.8, 7.4 Hz).

### Reference Example 174

### Methyl 2-[2-(hydroxymethyl)-1-benzothiophen-7-yl]pyridine-4-carboxylate

[7-(4,4,5,5-Tetramethyl-1,3,2-dioxaborolan-2-yl)-1-benzothiophen-2-yl]methanol obtained in Reference Example 172 and methyl 2-bromopyridine-4-carboxylate were used in the same manner as in Reference Example 4 to obtain the titled compound. 47% yield, crude oily substance. The product was used, without further purification, in the following reaction.

¹H-NMR (CDCl₃) δ: 2.40 (1H, br s), 4.01 (3H, s), 4.97 (2H, s), 7.29 (1H, s), 7.49 (1H, t, J=7.7 Hz), 7.75-7.87 (2H, m), 7.91-8.00 (1H, m), 8.52 (1H, s), 8.94 (1H, d, J=4.9 Hz).

### Reference Example 175

### Methyl 2-[2-(hydroxymethyl)-3-methyl-1-benzothiophen-7-yl]pyridine-4-carboxylate

[3-Methyl-7-(4,4,5,5-tetramethyl-1,3,2-dioxaborolan-2-yl)-1-benzothiophen-2-yl]methanol obtained in Reference Example 173 and methyl 2-bromopyridine-4-carboxylate were used in the same manner as in Reference Example 4 to obtain the titled compound. 65% yield, crude oily substance. The product was used, without further purification, in the following reaction.

¹H-NMR (CDCl₃) δ: 2.44 (3H, s), 4.01 (3H, s), 4.95 (2H, br s), 7.54 (1H, t, J=7.8 Hz), 7.74-7.86 (2H, m), 7.98 (1H, d, J=7.6 Hz), 8.52 (1H, s), 8.95 (1H, d, J=4.9 Hz).

### Reference Example 176

### Ethyl 4-[2-(hydroxymethyl)-1-benzothiophen-7-yl]benzoate

(7-Bromo-1-benzothiophen-2-yl)methanol obtained in Reference Example 162 and [4-(ethoxycarbonyl)phenyl]boronic acid were used in the same manner as in Reference Example 4 to obtain the titled compound. 99% yield, oily substance.

¹H-NMR (CDCl₃) δ: 1.42 (3H, t, J=7.2 Hz), 2.26 (1H, br s), 4.41 (2H, q, J=7.2 Hz), 4.92 (2H, d, J=4.2 Hz), 7.28 (1H, s), 7.31-7.38 (1H, m), 7.41-7.48 (1H, m), 7.73 (1H, dd, J=8.0, 1.1 Hz), 7.77 (2H, d, J=8.7 Hz), 8.14 (2H, d, J=8.7 Hz).

### Reference Example 177

### 3-[2-(Hydroxymethyl)-1-benzothiophen-7-yl]benzoic acid

4 N sodium hydroxide aqueous solution (25 ml, 100 mmol) was added to a THF (100 mL)-ethanol (100 mL) mixed solution of ethyl 3-[2-(hydroxymethyl)-1-benzothiophen-7-yl]benzoate (12.0 g, 38.4 mmol) obtained in Reference Example 168, and the mixture was stirred for 2 hours at 60° C. After being allowed to cool to room temperature, the reaction solution was neutralized with the addition of 1 N hydrochloric acid, diluted with water, and then extracted with ethyl acetate. The organic layer was washed with brine, dried over anhydrous magnesium sulfate, and then concentrated at reduced pressure. The residue was crystallized using ethyl acetate-hexane to give 10.8 g of the titled compound (yield 99%) in the form of pale yellow crystals.

¹H-NMR (DMSO-d₆) δ: 4.75 (2H, s), 5.66 (1H, br s), 7.35-7.39 (1H, m), 7.41 (1H, dd, J=1.2, 7.5 Hz), 7.49 (1H, t, J=7.5 Hz), 7.68 (1H, t, J=7.8 Hz), 7.82 (1H, dd, J=0.9, 7.8 Hz), 7.92-7.98 (1H, m), 7.99-8.06 (1H, m), 8.28-8.32 (1H, m), 13.17 (1H, br s).

### Reference Example 178

### 3-[2-(Hydroxymethyl)-1-benzothiophen-7-yl]-N-(2-methoxyethyl)benzamide

A mixed solution of 3-[2-(hydroxymethyl)-1-benzothiophen-7-yl]benzoic acid (10.8 g, 38.0 mmol) obtained in Reference Example 177, 2-methoxyethanamine (3.42 g, 45.6 mmol), WSC (10.9 g, 57.0 mmol), HOBt (8.73 g, 57.0 mmol), and triethylamine (5.77 g, 57.0 mmol) in DMF (150 mL) was stirred for 15 hours at room temperature. The reaction solution was diluted with water and extracted with ethyl acetate. The extract was washed with water and brine, and was dried over anhydrous magnesium sulfate, and the solvent was then distilled off at reduced pressure. The residue was purified by silica gel column chromatography (hexane-ethyl acetate=90:10-0/100) to give 10.9 g of the titled compound (yield 84%) in the form of a colorless oily substance.

¹H-NMR (DMSO-d₆) δ: 2.19 (1H, t, J=6.0 Hz), 3.38 (3H, s), 3.53-3.61 (2H, m), 3.63-3.72 (2H, m), 4.92 (2H, d, J=6.0 Hz), 6.62 (1H, br s), 7.28-7.32 (1H, m), 7.35 (1H, dd, J=1.2, 7.2 Hz), 7.45 (1H, t, J=7.5 Hz), 7.54 (1H, t, J=7.8 Hz), 7.73 (1H, dd, J=1.2, 7.8 Hz), 7.79-7.88 (2H, m), 8.06-8.11 (1H, m).

### Reference Example 179

### Ethyl 3-[2-(bromomethyl)-1-benzothiophen-7-yl]benzoate

Phosphorus tribromide (0.7 mL, 7.39 mmol) was added dropwise while cooled on ice to a diethyl ether (20 mL) solution of ethyl 3-[2-(hydroxymethyl)-1-benzothiophen-7-yl]benzoate (2.2 g, 7.04 mmol) obtained in Reference Example 168, and the mixture was stirred for 2 hours at room temperature. The reaction solution was cooled on ice and was quenched with the addition of water, and was then extracted with ethyl acetate. The extract was washed with water and dried over anhydrous magnesium sulfate, and the solvent was then distilled off at reduced pressure. The residue was purified by silica gel column chromatography (hexane:ethyl acetate=10:1) to give 1.91 g of the titled compound (yield 72%) in the form of crystals.

Melting point: 96-97° C.

¹H NMR (CDCl₃) δ: 1.41 (3H, t, J=7.2 Hz), 4.41 (2H,q, J=7.2 Hz), 4.77 (2H, s), 7.38-7.45 (2H, m), 7.46 (1H, t, J=7.5 Hz), 7.54 (1H, t, J=7.8 Hz), 7.73 (1H, d, J=7.5 Hz), 7.90 (1H, d, J=7.8 Hz), 8.10 (1H, d, J=7.5 Hz), 8.35 (1H, s).

### Reference Example 180

### Ethyl 3-[2-(bromomethyl)-3-methyl-1-benzothiophen-7-yl]benzoate

Phosphorus tribromide (1.81 mL, 19.3 mmol) was added dropwise while cooled on ice to a diethyl ether (100 mL) solution of ethyl 3-[2-(hydroxymethyl)-3-methyl-1-benzothiophen-7yl]benzoate (6.00 g, 18.4 mmol) obtained in Reference Example 169, and the mixture was then stirred for 1 hour at the same temperature. The reaction solution was poured into ice, and the mixture was extracted with ethyl acetate. The combined organic layers were washed with saturated sodium bicarbonate aqueous solution, dried over anhydrous magnesium sulfate, and then concentrated at reduced pressure. The residue was purified by silica gel column chromatography (hexane-ethyl acetate 100:0→88:12) to give 3.36 g of the titled compound (yield 47%) in the form of an oily substance.

¹H-NMR (CDCl₃) δ: 1.41 (3H, t, J=7.1 Hz), 2.43 (3H, s), 4.42 (2H, q, J=7.2 Hz), 4.78 (2H, s), 7.39-7.44 (1H, m), 7.50 (1H, t, J=7.6 Hz), 7.57 (1H, t, J=7.7 Hz), 7.70 (1H, dd, J=7.9, 1.1 Hz), 7.88-7.93 (1H, m), 8.06-8.13 (1H, m), 8.35 (1H, t, J=1.7 Hz).

### Reference Example 181

### Methyl 2-[2-(bromomethyl)-1-benzothiophen-7-yl]pyridine-4-carboxylate

Phosphorus tribromide (0.317 mL, 3.37 mmol) was added dropwise while cooled on ice to a THF (15 mL) solution of methyl 2-[2-(hydroxymethyl)-1-benzothiophen-7-yl]pyridine-4-carboxylate (0.96 g, 3.21 mmol) obtained in Reference Example 174, and the mixture was then stirred for 1 hour at the same temperature. The reaction solution was poured into ice and 1N sodium hydroxide aqueous solution, and the mixture was extracted with ethyl acetate. The combined organic layers were washed with water, dried over anhydrous magnesium sulfate, and then concentrated at reduced pressure. The residue was purified by silica gel column chromatography (hexane-ethyl acetate 100:0→90:10) to give 353 mg of the titled compound (yield 30%) in the form of an oily substance.

¹H-NMR (CDCl₃) δ: 4.02 (3H, s), 4.83 (2H, s), 7.42 (1H, s), 7.51 (1H, t, J=7.8 Hz), 7.78-7.87 (2H, m), 8.01 (1H, d, J=7.6 Hz), 8.54 (1H, s), 8.97 (1H, d, J=4.9 Hz).

### Reference Example 182

### Methyl 2-[2-(bromomethyl)-3-methyl-1-benzothiophen-7-yl]pyridine-4-carboxylate

Methyl 2-[2-(hydroxymethyl)-3-methyl-1-benzothiophen-7-yl]pyridine-4-carboxylate obtained in Reference Example 175 was used in the same manner as in Reference Example 181 to obtain the titled compound. The resulting crude product was used, without further purification, in the following reaction.

### Reference Example 183

### Ethyl 4-[2-(bromomethyl)-1-benzothiophen-7-yl]benzoate

Ethyl 4-[2-(hydroxymethyl)-1-benzothiophen-7-yl]benzoate obtained in Reference Example 176 was used in the same manner as in Reference Example 180 to obtain the titled compound. Yield: 82%.

¹H-NMR (CDCl₃) δ: 1.43 (3H, t, J=7.2 Hz), 4.42 (2H, q, J=7.2 Hz), 4.77 (2H, s), 7.37-7.42 (2H, m), 7.43-7.51 (1H, m), 7.70-7.82 (3H, m), 8.18 (2H, d, J=8.7 Hz).

### Reference Example 184

### 2-(Bromomethyl)-7-chloro-4-fluoro-1-benzothiophene

(7-Chloro-4-fluoro-1-benzothiophen-2-yl)methanol obtained in Reference Example 165 was used in the same manner as in Reference Example 179 to obtain the titled compound. Yield: 84%. Melting point: 89-90° C.

¹H-NMR (CDCl₃) δ: 4.75 (2H, s), 6.98 (1H, dd, J=9.3, 8.4 Hz), 7.23-7.29 (1H, m), 7.44 (1H, s).

### Reference Example 185

### Ethyl 3-[2-(bromomethyl)-4-fluoro-1-benzothiophen-7-yl]benzoate

Phosphorus tribromide (0.227 mL, 2.40 mmol) was added while cooled on ice to a diethyl ether (50 mL) solution of ethyl 3-[4-fluoro-2-(hydroxymethyl)-1-benzothiophen-7yl]benzoate (1.20 g, 3.63 mmol) synthesized in Reference Example 170, and the mixture was stirred for 1 hour at the same temperature. The addition of water to the reaction solution was followed by extraction with diisopropyl ether. The organic layer was washed with water and saturated sodium bicarbonate aqueous solution, dried over magnesium sulfate, filtered, and concentrated at reduced pressure to give 600 mg of the titled compound (yield 42%). Melting point: 139-140° C.

¹H-NMR (CDCl₃) δ: 1.41 (3H, t, J=7.1 Hz), 4.41 (2H, q, J=7.1 Hz), 4.75 (2H, s), 7.12 (1H, dd, J=9.3, 8.1 Hz), 7.33 (1H, dd, J=8.1, 4.8 Hz), 7.50 (1H, s), 7.56 (1H, t, J=7.8 Hz), 7.84 (1H, d, J=7.5 Hz), 8.09 (1H, d, J=7.8 Hz), 8.30 (1H, d, J=1.8 Hz).

### Reference Example 186

### Ethyl 3-[2-(bromomethyl)-4-chloro-1-benzothiophen-7-yl]benzoate

Ethyl 3-[4-chloro-2-(hydroxymethyl)-1-benzothiophen-7-yl]benzoate synthesized in Reference Example 171 was used in the same manner as in Reference Example 185 to obtain the titled compound. Yield: 75%. Melting point: 143-144° C.

¹H-NMR (CDCl₃) δ: 1.41 (3H, t, J=7.2 Hz), 4.42 (2H, q, J=7.2 Hz), 4.77 (2H, s), 7.31 (1H, d, J=8.1 Hz), 7.45 (1H, d, J=7.8 Hz), 7.53-7.58 (2H, m), 7.85 (1H, d, J=6.9 Hz), 8.10 (1H, d, J=6.9 Hz), 8.30 (1H, t, J=1.5 Hz).

### Reference Example 187

### 3-[2-(Bromomethyl)-1-benzothiophen-7-yl]-N-(2-methoxyethyl)benzamide

Phosphorus tribromide (3.2 mL, 33.5 mmol) was added while cooled on ice to an ether (120 mL) solution of 3-[2-(hydroxymethyl)-1-benzothiophen-7-yl]-N-(2-methoxyethyl)benzamide (10.9 g, 31.9 mmol) obtained in Reference Example 178. The reaction solution was warmed to room temperature and stirred for 2 hours. The addition of saturated aqueous sodium bicarbonate while cooled on ice to the reaction solution was followed by extraction with ethyl acetate. The extract was washed with brine and dried over anhydrous magnesium sulfate, and the solvent was then distilled off at reduced pressure. The residue was purified by silica gel column chromatography (hexane-ethyl acetate=90:10-0/100) to give 12.4 g of the titled compound (yield 96%) in the form of a pale yellow oily substance.

¹H-NMR (DMSO-d₆) δ: 3.40 (3H, s), 3.54-3.62 (2H, m), 3.64-3.73 (2H, m), 4.77 (2H, s), 6.59 (1H, br s), 7.36-7.42 (2H, m), 7.46 (1H, t, J=7.5 Hz), 7.57 (1H, t, J=7.8 Hz), 7.73 (1H, dd, J=1.2, 7.8 Hz), 7.80-7.89 (2H, m), 8.06-8.11 (1H, m).

### Reference Example 188

### Methyl[(2-bromophenyl)sulfanyl]acetate

2-Bromobenzenethiol (10 g, 52.89 mmol) was added to a mixture of methyl bromoacetate (6 mL, 63.47 mmol) and pyridine (5.13 mL, 63.47 mmol) in DMSO (130 mL), and the mixture was stirred for 2 hours and 30 min at room temperature. The reaction solution was diluted with ethyl acetate and was then washed with water and saturated brine. The resulting organic layer was dried over anhydrous sodium sulfate, and the solvent was then distilled off at reduced pressure. The residue was purified by silica gel column chromatography (hexane:ethyl acetate=90:10→80:20) to give 12.8 g of the titled compound (yield 93%). Oily substance.

¹H-NMR (CDCl₃) δ: 3.70 (2H, s), 3.73 (3H, s), 7.05-7.12 (1H, m), 7.30 (1H, dd, J=7.4, 1.4 Hz), 7.35-7.40 (1H, m), 7.56 (1H, dd, J7.7, 1.4 Hz).

### Reference Example 189

### [(2-Bromophenyl)sulfanyl]acetic acid

2 N sodium hydroxide aqueous solution (49 mL) was added to a THF solution (150 mL) of methyl[(2-bromophenyl)sulfanyl]acetate (12.8 g, 49.02 mmol) obtained in Reference Example 188, and the mixture was stirred over night at room temperature. The reaction solution was concentrated at reduced pressure, and the residue was made acidic with the addition of 1 N hydrochloric acid and was extracted with ethyl acetate. The organic layer was washed with saturated brine and dried over sodium sulfate. The reaction solution was filtered, and the solvent was then distilled off at reduced pressure to give 10.8 g of the titled compound (yield 89%).

¹H-NMR (CDCl₃) δ: 3.73 (2H, s), 7.06-7.13 (1H, m), 7.26-7.32 (1H, m), 7.35-7.41 (1H, m), 7.57 (1H, dd, J=8.0, 1.4 Hz), (1H unconfirmed).

### Reference Example 190

### 7-Bromo-2-[3-(trifluoromethyl)benzyl]-1-benzothiophene

A mixture of [(2-bromophenyl)sulfanyl]acetic acid (15.1 g, 61.1 mmol) obtained in Reference Example 189 and thionyl chloride (40 mL) was heated to reflux for 2 hours. The reaction solution was concentrated at reduced pressure, the residue was then diluted with chlorobenzene (200 mL), and aluminum chloride (20.4 g, 152.7 mmol) was added while cooled on ice. The mixture was stirred for 1 hour at the same temperature, the ice bath was then removed, and the reaction solution was stirred for 16 hours at room temperature. The reaction solution was poured into ice water and was extracted with ethyl acetate. The extract was washed with saturated brine and dried over anhydrous sodium sulfate, and the solvent was then distilled off at reduced pressure to give 7-bromo-1-benzothiophen-3(2H)-one crude product (20.4 g). A toluene solution (220 mL) of the resulting 7-bromo-1-benzothiophen-3(2H)-one crude product (15.0 g), 3-(trifluoromethyl)benzaldehyde (6.0 mL, 45.0 mmol), and piperidine (0.1 mL, cat.) was heated to reflux for 2 hours. The reaction solution was allowed to cool to room temperature and was filtered. The filtrate was washed with water and saturated brine, and was dried over anhydrous sodium sulfate. The material was filtered, the solvent was then distilled off at reduced pressure, and the residue was suspended in hexane and filtered to give 7-bromo-2-[[3-(trifluoromethyl)phenyl]methylidene]-1-benzothiophen-3 (2H)-one crude product (12.3 g) in the form of residue. Triethylsilane (12.7 mL, 79.6 mmol) was added while cooled on ice to a toluene solution (180 mL) of the 7-bromo-2-[[3-(trifluoromethyl)phenyl]methylidene]-1-benzothiophen-3(2H)-one crude product (12.3 g) and trifluoromethanesulfonic acid (7.0 mL, 79.6 mmol). Triethylsilane was added dropwise, the ice bath was removed, and the reaction solution was stirred for 3 hours at room temperature. The addition of water to the reaction solution was followed by extraction with ethyl acetate. The extract was washed with saturated brine and dried over anhydrous sodium sulfate, and the solvent was then distilled off at reduced pressure. The residue was purified by silica gel column chromatography (hexane:ethyl acetate=95:5) to give 10.5 g of the titled compound (yield 63%, 3 steps).

¹H-NMR (CDCl₃) δ: 4.29 (2H, s), 7.12 (1H, s), 7.16-7.24 (1H, m), 7.39-7.58 (5H, m) 7.63 (1H, d, J=8.0 Hz).

### Reference Example 191

### 7-Bromo-2-(3-chloro-5-fluorobenzyl)-1-benzothiophene

[(2-Bromophenyl)sulfanyl]acetic acid obtained in Reference Example 189 was used in the same manner as in Reference Example 190 to obtain the titled compound. Yield: 93%.

¹H-NMR (CDCl₃) δ: 4.19 (2H, s), 6.90 (1H, dt, J=8.9, 1.9 Hz), 7.00 (1H, dt, J=8.3, 2.2 Hz), 7.08 (1H, s), 7.14 (1H, s), 7.18-7.24 (1H, m), 7.43 (1H, dd, J=7.7, 0.8 Hz), 7.63 (1H, d, J=8.0 Hz).

### Reference Example 192

### 2-[2-(3-Chloro-5-fluorobenzyl)-1-benzothiophen-7-yl]-4,4,5,5-tetramethyl-1,3,2-dioxaborolane

A DMF (6.0 mL) solution of the 7-bromo-2-(3-chloro-5-fluorobenzyl)-1-benzothiophene (300 mg, 0.844 mmol) obtained in Reference Example 191, bis(pinacolato)diboron (257 mg, 1.01 mmol), [1,1-bis(diphenylphosphino)ferrocene]dichloropalladium (34.4 mg, 0.042 mmol), and potassium acetate (248 mg, 2.53 mmol) was stirred over night at 80° C. in a nitrogen atmosphere. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate:hexane=1:19) to give 274 mg of the titled compound (yield 81%) in solid form.

¹H-NMR (CDCl₃) δ: 1.39 (12H, s), 4.19 (2H, s), 6.84-6.94 (1H, m), 6.94-7.00 (1H, m), 7.03 (1H, s), 7.08 (1H, s), 7.13-7.20 (1H, m), 7.33 (1H, t, J=7.5 Hz), 7.73-7.81 (1H, m).

### Reference Example 193

### 4,4,5,5-tetramethyl-2-[2-[3-(trifluoromethyl)benzyl]-1-benzothiophen-7-yl]-1,3,2-dioxaborolane

7-Bromo-2-[3-(trifluoromethyl)benzyl]-1-benzothiophene obtained in Reference Example 190 was used in the same manner as in Reference Example 192 to obtain the titled compound in solid form. Yield: 77%.

¹H-NMR (CDCl₃) δ: 1.38 (12H, s), 4.28 (2H, s), 6.99 (1H, s), 7.29-7.36 (1H, m), 7.37-7.54 (3H, m), 7.57 (1H, s), 7.71-7.79 (2H, m).

### Reference Example 194

### 7-Chloro-4-fluoro-2-[3-(methylsulfonyl)benzyl]-1-benzothiophene

A mixture of 2-(bromomethyl)-7-chloro-4-fluoro-1-benzothiophene (0.72 g, 2.59 mmol) obtained in Reference Example 184, [3-(methylsulfonyl)phenyl]boronic acid (0.57 g, 2.85 mmol), tetrakis(triphenylphosphine)palladium (0) (0.15 g, 0.13 mmol), sodium carbonate (0.55 g, 5.15 mmol), water (10 mL), and dimethoxyethane (20 mL) was stirred for 1.5 hours at 70° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=4:1-1:1) to give 0.69 g of the titled compound (yield 75%) in the form of an oily substance.

¹H NMR (CDCl₃) δ: 3.06 (3H, s), 4.33 (2H, s), 6.97 (1H, t, J=8.9 Hz), 7.15-7.30 (2H, m), 7.50-7.65 (2H, m), 7.80-7.90 (2H, m).

### Reference Example 195

### 1-Bromo-3-fluoro-5-(methylsulfonyl)benzene

While cooled on ice, m-chloroperbenzoic acid (70%, 7.8 g, 31.6 mmol) was added a little at a time to an ethyl acetate (70 mL) solution of 1-bromo-3-fluoro-5-(methylsulfanyl)benzene (3.5 mL, 15.8 mmol). The mixture was stirred for 1 hour at room temperature, and the reaction solution was then diluted with water and extracted with ethyl acetate. The extract was washed with saturated aqueous sodium bicarbonate, dried over anhydrous magnesium sulfate, and then allowed to flow through basic silica gel, and the solvent was distilled off at reduced pressure to give 3.92 g of the titled compound (yield 98%) in the form of crystals.

¹H NMR (CDCl₃) δ: 3.09 (3H, s), 7.54 (1H, d, J=7.8 Hz), 7.61 (1H, d, J=7.8 Hz), 7.90 (1H, s).

### Reference Example 196

### 7-Chloro-2-[3-fluoro-5-(methylsulfonyl)benzyl]-1-benzothiophene

A mixture of 1-bromo-3-fluoro-5-(methylsulfonyl)benzene (1.0 g, 3.95 mmol) obtained in Reference Example 195, bis(pinacolato)diborane (1.20 g, 4.74 mmol), [1,1′-bis(diphenylphosphino)ferrocene]dichloropalladium (II) complex with dichloromethane (0.16 g, 0.20 mmol), and potassium acetate (1.16 mg, 11.9 mmol) in DMSO (15 mL) was stirred for 2 hours at 75° C. The reaction solution was diluted with water and extracted with ethyl acetate. The extract was washed with water and dried over anhydrous magnesium sulfate, and the solvent was then distilled off at reduced pressure. A mixture of the residue, 2-(bromomethyl)-7-chloro-1-benzothiophene (0.69 g, 2.64 mmol), tetrakis(triphenylphosphine)palladium (0) (0.15 g, 0.13 mmol), sodium carbonate (0.56 g, 5.28 mmol), water (10 mL), and dimethoxyethane (25 mL) was stirred for 14 hours at 75° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=3:2) to give 0.70 g of the titled compound (yield 86%) in the form of an oily substance.

¹H NMR (CDCl₃) δ: 3.07 (3H, s), 4.32 (2H, s), 7.12 (1H, s), 7.20-7.40 (3H, m), 7.55 (1H, d, J=7.2 Hz), 7.60 (1H, t, J=8.4 Hz), 7.68 (1H, s).

### Reference Example 197

### Ethyl 3-[2-[3-(trifluoromethyl)benzyl]-1-benzothiophen-4-yl]benzoate

Phosphorus tribromide (126 μL, 1.34 mmol) was added to a diethyl ether solution (13 mL) of ethyl 3-[2-(hydroxymethyl)-1-benzothiophen-4-yl]benzoate (399 mg, 1.28 mmol) obtained in Reference Example 167, and the mixture was stirred for 1 hour and 30 min at room temperature. The reaction solution was diluted with water and extracted with ethyl acetate. The extract was washed with saturated brine and dried over anhydrous sodium sulfate, and the solvent was then distilled off at reduced pressure to give ethyl 3-[2-(bromomethyl)-1-benzothiophen-4-yl]benzoate crude product (448 mg). A mixture of the resulting ethyl 3-[2-(bromomethyl)-1-benzothiophen-4-yl]benzoate crude product, [3-(trifluoromethyl)phenyl]boronic acid (272 mg, 1.43 mmol), and tetrakis(triphenylphosphine)palladium (0) (55 mg, 0.048 mmol) in 2 M sodium carbonate aqueous solution (2.4 mL)-1,2-dimethoxyethane (15 mL) was heated to reflux for 12 hours in a nitrogen atmosphere. The reaction solution was diluted with saturated brine and ethyl acetate, and was filtered using celite. The resulting filtrate was extracted with ethyl acetate. The combined organic layers were dried over anhydrous sodium sulfate, and then concentrated at reduced pressure, and the residue was purified by silica gel column chromatography (hexane-ethyl acetate 95:5→80:20) to give 392 mg of the titled compound (yield 70%, 2 steps).

¹H-NMR (CDCl₃) δ: 1.40 (3H, t, J=7.1 Hz), 4.26 (2H, s), 4.40 (2H, q, J=7.2 Hz), 7.14 (1H, d, J=0.8 Hz), 7.29-7.53 (6H, m), 7.53-7.58 (1H, m), 7.70-7.78 (2H, m), 8.05-8.10 (1H, m), 8.21-8.24 (1H, m).

### Reference Example 198

### Ethyl 3-[2-(3-chloro-4-fluorobenzyl)-1-benzothiophen-4-yl]benzoate

Ethyl 3-[2-(hydroxymethyl)-1-benzothiophen-4-yl]benzoate obtained in Reference Example 167 and phosphorus tribromide were used in the same manner as in Reference Example 197 to synthesize an ethyl 3-[2-(bromomethyl)-1-benzothiophen-4-yl]benzoate crude product, and the resulting ethyl 3-[2-(bromomethyl)-1-benzothiophen-4-yl]benzoate crude product and (3-chloro-4-fluorophenyl)boronic acid were used to obtain the titled compound. Yield: 64% (2 steps).

¹H-NMR (CDCl₃) δ: 1.40 (3H, t, J=7.1 Hz), 4.15 (2H, s), 4.41 (2H, q, J=7.1 Hz), 7.01-7.16 (3H, m), 7.25-7.39 (3H, m), 7.55 (1H, t, J=7.7 Hz), 7.70-7.78 (2H, m), 8.05-8.10 (1H, m), 8.21-8.25 (1H, m).

### Reference Example 199

### Ethyl 3-[2-(3-chloro-5-fluorobenzyl)-1-benzothiophen-4-yl]benzoate

Ethyl 3-[2-(hydroxymethyl)-1-benzothiophen-4-yl]benzoate obtained in Reference Example 167 and phosphorus tribromide were used in the same manner as in Reference Example 197 to synthesize an ethyl 3-[2-(bromomethyl)-1-benzothiophen-4-yl]benzoate crude product, and the resulting ethyl 3-[2-(bromomethyl)-1-benzothiophen-4-yl]benzoate crude product and (3-chloro-5-fluorophenyl)boronic acid were used to obtain the titled compound. Yield: 61% (2 steps).

¹H-NMR (CDCl₃) δ: 1.40 (3H, t, J=7.1 Hz), 4.17 (2H, s), 4.41 (2H, q, J=7.1 Hz), 6.83-6.90 (1H, m), 6.92-6.98 (1H, m), 7.04 (1H, s), 7.15 (1H, s), 7.30-7.34 (2H, m), 7.55 (1H, t, J=7.7 Hz), 7.70-7.79 (2H, m), 8.06-8.12 (1H, m), 8.21-8.25 (1H, m).

### Reference Example 200

### Ethyl 3-[2-[3-(trifluoromethyl)benzyl]-1-benzothiophen-7-yl]benzoate

A mixture of ethyl 3-[2-(bromomethyl)-1-benzothiophen-7-yl]benzoate (1.61 g, 4.29 mmol) obtained in Reference Example 179, [3-(trifluoromethyl)phenyl]boronic acid (0.98 g, 5.15 mmol), tetrakis(triphenylphosphine)palladium (0) (0.15 g, 0.13 mmol), sodium carbonate (0.91 g, 8.58 mmol), water (10 mL), and dimethoxyethane (30 mL) was stirred for 14 hours at 80° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=10:1) to give 1.36 g of the titled compound (yield 72%) in the form of an oily substance.

¹H NMR (CDCl₃) δ: 1.39 (3H, t, J=6.9 Hz), 4.27 (2H, s), 4.93 (2H, d, J=6.9 Hz), 7.10 (1H, s), 7.32 (1H, d, J=8.1 Hz), 7.40-7.60 (6H, m), 7.68 (1H, d, J=7.5 Hz), 7.86 (1H, d, J=8.1 Hz), 8.06 (1H, d, J=6.3 Hz), 8.33 (1H, s).

### Reference Example 201

### Ethyl 3-[2-[3-(methylsulfonyl)benzyl]-1-benzothiophen-7-yl]benzoate

A mixture of ethyl 3-[2-(bromomethyl)-1-benzothiophen-7-yl]benzoate (0.70 g, 1.87 mmol) obtained in Reference Example 179, [3-(methylsulfonyl)phenyl]boronic acid (0.45 g, 2.24 mmol), tetrakis(triphenylphosphine)palladium (0) (108 mg, 0.093 mmol), sodium carbonate (0.40 g, 3.73 mmol), water (6 mL), and dimethoxyethane (15 mL) was stirred for 1 hour at 75° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=1:1) to give 0.65 g of the titled compound (yield 77%) in the form of an oily substance.

¹H NMR (CDCl₃) δ: 1.39 (3H, t, J=7.2 Hz), 3.03 (3H, s), 4.31 (2H, s), 4.39 (2H, q, J=7.2 Hz), 7.14 (1H, s), 7.33 (1H, d, J=7.5 Hz), 7.43 (1H, t, J=7.2 Hz), 7.47-7.60 (4H, m), 7.69 (1H, d, J=8.1 Hz), 7.78-7.90 (2H, m), 8.06 (1H, d, J=7.5 Hz), 7.33 (1H, s).

### Reference Example 202

### Ethyl 3-[2-[3-fluoro-5-(trifluoromethyl)benzyl]-1-benzothiophen-7-yl]benzoate

A mixture of ethyl 3-[2-(bromomethyl)-1-benzothiophen-7-yl]benzoate (1.0 g, 2.66 mmol) obtained in Reference Example 179, [3-fluoro-5-(trifluoromethyl)phenyl]boronic acid (0.66 g, 3.20 mmol), tetrakis(triphenylphosphine)palladium (0) (154 mg, 1.33 mmol), sodium carbonate (0.56 g, 5.33 mmol), water (10 mL), and dimethoxyethane (20 mL) was stirred for 1 hour at 70° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=10:1-4:1) to give 0.92 g of the titled compound (yield 75%) in the form of an oily substance.

¹H NMR (CDCl₃) δ: 1.39 (3H, t, J=7.2 Hz), 4.27 (2H, s), 4.40 (2H, q, J=7.2 Hz), 7.14 (1H, s), 7.18 (2H, t, J=8.1 Hz), 7.30-7.40 (2H, m), 7.44 (1H, t, J=7.5 Hz), 7.54 (1H, t, J=7.8 Hz), 7.71 (1H, d, J=7.8 Hz), 7.87 (1H, d, J=7.8 Hz), 8.08 (1H, d, J=8.4 Hz), 8.34 (1H, s).

### Reference Example 203

### Ethyl 3-[4-fluoro-2-[3-(methylsulfonyl)benzyl]-1-benzothiophen-7-yl]benzoate

A mixture of the 7-chloro-4-fluoro-2-[3-(methylsulfonyl)benzyl]-1-benzothiophene (0.67 g, 1.89 mmol) obtained in Reference Example 194, [3-(ethoxycarbonyl)phenyl]boronic acid (0.44 g, 2.27 mmol), palladium acetate (12.7 mg, 0.057 mmol), 2-dicyclohexylphosphino-2′,4′,6′-triisopropyl biphenyl (X-Phos) (53.9 mg, 0.11 mmol), and potassium phosphate (0.80 g, 3.78 mmol) in THF (15 mL) was stirred for 28 hours at 75° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=3:1-3:2) to give 0.78 g of the titled compound (yield 88%) in the form of an oily substance.

¹H NMR (CDCl₃) δ: 1.40 (3H, t, J=7.2 Hz), 3.04 (3H, s), 4.31 (2H, s), 4.40 (2H, q, J=7.2 Hz), 7.11 (1H, t, J=9.0 Hz), 7.20-7.35 (2H, m), 7.45-7.60 (3H, m), 7.75-7.90 (3H, m), 8.06 (1H, d, J=7.5 Hz), 8.28 (1H, s).

### Reference Example 204

### Ethyl 3-[2-(3-chloro-4-fluorobenzyl)-1-benzothiophen-7-yl]benzoate

Ethyl 3-[2-(bromomethyl)-1-benzothiophen-7-yl]benzoate obtained in Reference Example 179 and (3-chloro-4-fluorophenyl)boronic acid were used in the same manner as in Reference Example 200 to obtain the titled compound. Yield: 81%.

¹H-NMR (CDCl₃) δ: 1.39 (3H, t, J=7.1 Hz), 4.16 (2H, s), 4.40 (2H, q, J=7.2 Hz), 7.02-7.09 (2H, m), 7.09-7.16 (2H, m), 7.27-7.35 (2H, m), 7.39-7.46 (1H, m), 7.54 (1H, t, J=7.7 Hz), 7.66-7.71 (1H, m), 7.84-7.89 (1H, m), 8.04-8.09 (1H, m), 8.32-8.35 (1H, m).

### Reference Example 205

### Ethyl 3-[2-(3,4-difluorobenzyl)-1-benzothiophen-7-yl]benzoate

Ethyl 3-[2-(bromomethyl)-1-benzothiophen-7-yl]benzoate obtained in Reference Example 179 and (3,4-difluorophenyl)boronic acid were used in the same manner as in Reference Example 200 to obtain the titled compound. Yield: 81%.

¹H-NMR (CDCl₃) δ: 1.39 (3H, t, J=7.1 Hz), 4.17 (2H, s), 4.40 (2H, q, J=7.1 Hz), 6.94-7.02 (1H, m), 7.02-7.14 (3H, m), 7.30-7.35 (1H, m), 7.39-7.46 (1H, m), 7.54 (1H, t, J=7.7 Hz), 7.68 (1H, dd, J=7.7, 1.1 Hz), 7.83-7.89 (1H, m), 8.04-8.09 (1H, m), 8.31-8.35 (1H, m).

### Reference Example 206

### Ethyl 3-[2-(3-chloro-5-fluorobenzyl)-1-benzothiophen-7-yl]benzoate

Ethyl 3-[2-(bromomethyl)-1-benzothiophen-7-yl]benzoate obtained in Reference Example 179 and (3,4-difluorophenyl)boronic acid were used in the same manner as in Reference Example 200 to obtain the titled compound. Yield: 79%.

¹H-NMR (CDCl₃) δ: 1.39 (3H, t, J=7.1 Hz), 4.18 (2H, s), 4.40 (2H, q, J=7.1 Hz), 6.85-6.91 (1H, m), 6.93-6.99 (1H, m), 7.06 (1H, s), 7.13 (1H, s), 7.31-7.35 (1H, m), 7.44 (1H, t, J=7.7 Hz), 7.54 (1H, t, J=7.7 Hz), 7.70 (1H, dd, J=8.0, 1.1 Hz), 7.85-7.90 (1H, m), 8.05-8.09 (1H, m), 8.32-8.35 (1H, m).

### Reference Example 207

### Ethyl 3-[2-(3,4-difluorobenzyl)-1-benzothiophen-4-yl]benzoate

Ethyl 3-[2-(hydroxymethyl)-1-benzothiophen-4-yl]benzoate obtained in Reference Example 167 and phosphorus tribromide were used in the same manner as in Reference Example 200 to synthesize an ethyl 3-[2-(bromomethyl)-1-benzothiophen-4-yl]benzoate crude product, and the resulting ethyl 3-[2-(bromomethyl)-1-benzothiophen-4-yl]benzoate crude product and (3,4-difluorophenyl)boronic acid were used to obtain the titled compound. Yield: 58%.

¹H-NMR (CDCl₃) δ: 1.41 (3H, t, J=7.1 Hz), 4.17 (2H, s), 4.41 (2H, q, J=7.1 Hz), 6.93-7.01 (1H, m), 7.01-7.10 (2H, m), 7.13 (1H, s), 7.29-7.40 (2H, m), 7.55 (1H, t, J=7.7 Hz), 7.70-7.80 (2H, m), 8.02-8.11 (1H, m), 8.23 (1H, t, J=1.8 Hz)

### Reference Example 208

### Ethyl 3-[3-methyl-2-[3-(trifluoromethyl)benzyl]-1-benzothiophen-7-yl]benzoate

Ethyl 3-[2-(bromomethyl)-3-methyl-1-benzothiophen-7-yl]benzoate obtained in Reference Example 180 and [3-(trifluoromethyl)phenyl]boronic acid were used in the same manner as in Reference Example 4 to obtain the titled compound. 57% yield, oily substance.

¹H-NMR (CDCl₃) δ: 1.38 (3H, t, J=7.2 Hz), 2.41 (3H, s), 4.25 (2H, s), 4.39 (2H, q, J=7.2 Hz), 7.33-7.41 (3H, m), 7.43-7.58 (4H, m), 7.67 (1H, dd, J=7.9, 1.1 Hz), 7.88 (1H, dt, J=7.7, 1.5 Hz), 8.07 (1H, dt, J=7.8, 1.4 Hz), 8.34 (1H, t, J=1.5 Hz).

### Reference Example 209

### Methyl 2-[2-[3-(trifluoromethyl)benzyl]-1-benzothiophen-7-yl]pyridine-4-carboxylate

Methyl 2-[2-(bromomethyl)-1-benzothiophen-7-yl]pyridine-4-carboxylate obtained in Reference Example 181 and [3-(trifluoromethyl)phenyl]boronic acid were used in the same manner as in Reference Example 4 to obtain the titled compound. Yield: 50%; melting point: 106-107° C. (ethyl acetate).

¹H-NMR (CDCl₃) δ: 4.00 (3H, s), 4.32 (2H, s), 7.10 (1H, s), 7.38-7.54 (4H, m), 7.58 (1H, s), 7.74-7.84 (2H, m), 7.93 (1H, d, J=7.6 Hz), 8.51 (1H, s), 8.93 (1H, d, J=4.9 Hz).

### Reference Example 210

### Methyl 2-[2-(3-chloro-5-fluorobenzyl)-1-benzothiophen-7-yl]pyridine-4-carboxylate

Methyl 2-[2-(bromomethyl)-1-benzothiophen-7-yl]pyridine-4-carboxylate obtained in Reference Example 181 and (3-chloro-5-fluorophenyl)boronic acid were used in the same manner as in Reference Example 4 to obtain the titled compound. Yield: 7%; melting point: 102-103° C. (ethyl acetate).

¹H-NMR (CDCl₃) δ: 4.01 (3H, s), 4.23 (2H, s), 6.90-7.01 (2H, m), 7.09-7.15 (2H, m), 7.49 (1H, t, J=7.8 Hz), 7.78-7.83 (2H, m), 7.95 (1H, d, J=7.6 Hz), 8.52 (1H, s), 8.94 (1H, dd, J=4.9, 0.8 Hz).

### Reference Example 211

### Methyl 2-[3-methyl-2-[3-(trifluoromethyl)benzyl]-1-benzothiophen-7-yl]pyridine-4-carboxylate

Methyl 2-[2-(bromomethyl)-3-methyl-1-benzothiophen-7-yl]pyridine-4-carboxylate obtained in Reference Example 182 and [3-(trifluoromethyl)phenyl]boronic acid were used in the same manner as in Reference Example 4 to obtain the titled compound. 7% yield, oily substance.

¹H-NMR (CDCl₃) δ: 2.41 (3H, s), 4.00 (3H, s), 4.31 (2H, s), 7.34-7.49 (3H, m), 7.49-7.60 (2H, m), 7.74-7.83 (2H, m), 7.96 (1H, d, J=7.6 Hz), 8.51 (1H, s), 8.92 (1H, d, J=4.9 Hz).

### Reference Example 212

### Ethyl 4-[2-[3-(trifluoromethyl)benzyl]-1-benzothiophen-7-yl]benzoate

Ethyl (4-[2-(bromomethyl)-1-benzothiophen-7-yl]benzoate obtained in Reference Example 183 and [3-(trifluoromethyl)phenyl]boronic acid were used in the same manner as in Reference Example 4 to obtain the titled compound. 72% yield, oily substance.

¹H-NMR (CDCl₃) δ: 1.41 (3H, t, J=7.2 Hz), 4.27 (2H, s), 4.41 (2H, q, J=7.2 Hz), 7.11 (1H, s), 7.33 (1H, dd, J=7.6, 1.1 Hz), 7.39-7.48 (3H, m), 7.52 (2H, d, J=12.5 Hz), 7.70 (1H, dd, J=8.0, 1.1 Hz), 7.75 (2H, d, J=8.3 Hz), 8.14 (2H, d, J=8.7 Hz).

### Reference Example 213

### Ethyl 6-[2-[3-(trifluoromethyl)benzyl]-1-benzothiophen-7-yl]pyridine-3-carboxylate

4,4,5,5-Tetramethyl-2-[2-[3-(trifluoromethyl)benzyl]-1-benzothiophen-7-yl]-1,3,2-dioxaborolane obtained in Reference Example 193 and ethyl 6-chloropyridine-3-carboxylate were used in the same manner as in Reference Example 4 to obtain the titled compound. 78% yield, oily substance.

¹H-NMR (CDCl₃) δ: 1.43 (3H, t, J=7.2 Hz), 4.32 (2H, s), 4.44 (2H, q, J=7.2 Hz), 7.10 (1H, s), 7.39-7.54 (4H, m), 7.58 (1H, s), 7.80 (1H, d, J=7.9 Hz), 7.90 (1H, d, J=6.8 Hz), 8.01 (1H, d, J=8.7 Hz), 8.39 (1H, dd, J=8.3, 2.3 Hz), 9.39 (1H, s).

### Reference Example 214

### Ethyl 3-[4-fluoro-2-[3-(trifluoromethyl)benzyl]-1-benzothiophen-7-yl]benzoate

A mixture of ethyl 3-[2-(bromomethyl)-4-fluoro-1-benzothiophen-7-yl]benzoate (210 mg, 0.53 mmol) synthesized in Reference Example 185, [3-(trifluoromethyl)phenyl]boronic acid (112 mg, 0.59 mmol), and tetrakis(triphenylphosphine)palladium (0) (31 mg, 0.027 mmol) in 2 N sodium carbonate aqueous solution (20 mL)-1,2-dimethoxyethane (20 mL) was heated to reflux for 16 hours. The reaction solution was diluted with saturated brine and ethyl acetate, and was filtered using celite. The resulting filtrate was extracted with ethyl acetate. The combined organic layers were dried over anhydrous sodium sulfate, and then concentrated at reduced pressure, and the residue was purified by silica gel column chromatography (hexane-ethyl acetate 100:0→20:80) to give 200 mg of the titled compound (yield 82%).

¹H-NMR (CDCl₃) δ: 1.39 (3H, t, J=7.1 Hz), 4.28 (2H, s), 4.39 (2H, q, J=7.1 Hz), 7.05-7.15 (1H, m), 7.22 (1H, d, J=0.8 Hz), 7.24-7.31 (1H, m), 7.39-7.62 (5H, m), 7.77-7.85 (1H, m), 8.03-8.10 (1H, m), 8.28 (1H, t, J=1.8 Hz).

### Reference Example 215

### Ethyl 3-[2-(3-chloro-5-fluorobenzyl)-4-fluoro-1-benzothiophen-7-yl]benzoate

Ethyl 3-[2-(bromomethyl)-4-fluoro-1-benzothiophen-7-yl]benzoate obtained in Reference Example 185 was used in the same manner as in Reference Example 214 to obtain the titled compound. Yield: 69%.

¹H-NMR (CDCl₃) δ: 1.38 (3H, t, J=7.2 Hz), 4.18 (2H, s), 4.39 (2H, q, J=7.2 Hz), 6.88 (1H, d, J=9.6 Hz), 6.97 (1H, d, J=8.4 Hz), 7.05 (1H, s), 7.10 (1H, dd, J=9.6, 8.4 Hz), 7.22-7.32 (2H, m), 7.53 (1H, t, J=7.8 Hz), 7.81 (1H, d, J=7.8 Hz), 8.06 (1H, d, J=7.8 Hz), 8.28 (1H, s).

### Reference Example 216

### Ethyl 3-[2-(3,4-difluorobenzyl)-4-fluoro-1-benzothiophen-7-yl]benzoate

Ethyl 3-[2-(bromomethyl)-4-fluoro-1-benzothiophen-7-yl]benzoate obtained in Reference Example 185 was used in the same manner as in Reference Example 214 to obtain the titled compound. Yield: 77%.

¹H-NMR (CDCl₃) δ: 1.39 (3H, t, J=7.2 Hz), 4.17 (2H, s), 4.39 (2H, q, J=7.2 Hz), 6.92-7.16 (4H, m), 7.20-7.31 (2H, m), 7.52 (1H, t, J=7.5 Hz), 7.80 (1H, d, J=9.3 Hz), 8.05 (1H, d, J=9.3 Hz), 8.27 (1H, s).

### Reference Example 217

### Ethyl 3-[4-chloro-2-(3,4-difluorobenzyl)-1-benzothiophen-7-yl]benzoate

Ethyl 3-[2-(bromomethyl)-4-chloro-1-benzothiophen-7-yl]benzoate obtained in Reference Example 186 was used in the same manner as in Reference Example 214 to obtain the titled compound. Yield: 72%; melting point: 124-125° C. (ethyl acetate-hexane).

¹H-NMR (CDCl₃) δ: 1.39 (3H, t, J=7.2 Hz), 4.18 (2H, s), 4.39 (2H, q, J=7.2 Hz), 6.93-7.18 (3H, m), 7.22-7.32 (2H, m), 7.42 (1H, d, J=7.8 Hz), 7.53 (1H, t, J=7.5 Hz), 7.80 (1H, d, J=8.4 Hz), 8.07 (1H, d, J=7.8 Hz), 8.27 (1H, s).

### Reference Example 218

### Ethyl 3-[4-chloro-2-(3-chloro-5-fluorobenzyl)-1-benzothiophen-7-yl]benzoate

Ethyl 3-[2-(bromomethyl)-4-chloro-1-benzothiophen-7-yl]benzoate obtained in Reference Example 186 was used in the same manner as in Reference Example 214 to obtain the titled compound. Yield: 66%.

¹H-NMR (CDCl₃) δ: 1.40 (3H, t, J=7.2 Hz), 4.19 (2H, s), 4.40 (2H, q, J=7.2 Hz), 6.89 (1H, d, J=8.4 Hz), 6.93-6.99 (1H, m), 7.06 (1H, s), 7.27 (1H, s), 7.33 (1H, s), 7.44 (1H, d, J=7.8 Hz), 7.55 (1H, t, J=7.5 Hz), 7.78-7.84 (1H, m), 8.05-8.10 (1H, m), 8.29 (1H, t, J=1.8 Hz).

### Reference Example 219

### Ethyl 5-[2-(3-chloro-5-fluorobenzyl)-1-benzothiophen-7-yl]pyridine-3-carboxylate

A mixture of the 7-bromo-2-(3-chloro-5-fluorobenzyl)-1-benzothiophene (500 mg, 1.41 mmol) obtained in Reference Example 191, ethyl 5-(4,4,5,5-tetramethyl-1,3,2-dioxaborolan-2-yl)pyridine-3-carboxylate (974 mg, 3.51 mmol), and tetrakis(triphenylphosphine)palladium (0) (195 mg, 0.169 mmol) in 2 N sodium carbonate aqueous solution (2.1 mL)-1,2-dimethoxyethane (10 mL) was stirred for 2 hours at 95° C. in a nitrogen atmosphere. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1:4) to give 416 mg of the titled compound (yield 69%) in the form of an oily substance.

¹H NMR (CDCl₃) δ: 1.42 (3H, t, J=7.0 Hz), 4.19 (2H, s), 4.45 (2H, q, J=7.0 Hz), 6.85-6.92 (1H, m), 6.94-7.01 (1H, m), 7.07 (1H, br s), 7.15 (1H, s), 7.31-7.37 (1H, m), 7.43-7.51 (1H, m), 7.72-7.79 (1H, m), 8.59 (1H, t, J=2.3 Hz), 9.07 (1H, d, J=2.3 Hz), 9.25 (1H, d, J=1.9 Hz).

### Reference Example 220

### Methyl 6-[2-(3-chloro-5-fluorobenzyl)-1-benzothiophen-7-yl]pyridine-2-carboxylate

A mixture of 2-[2-(3-chloro-5-fluorobenzyl)-1-benzothiophen-7-yl]-4,4,5,5-tetramethyl-1,3,2-dioxaborolane (274 mg, 0.680 mmol) obtained in Reference Example 192, methyl 6-bromopyridine-2-carboxylate (176 mg, 0.816 mmol), and tetrakis(triphenylphosphine)palladium (0) (94.2 mg, 0.082 mmol) in 2 N sodium carbonate aqueous solution (1.0 mL)-1,2-dimethoxyethane (5.4 mL) was stirred for 2 hours at 95° C. in a nitrogen atmosphere. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1:4) to give 220 mg of the titled compound (yield 79%) in the form of an oily substance.

¹H NMR (CDCl₃) δ: 4.06 (3H, s), 4.25 (2H, s), 6.93-7.02 (2H, m), 7.08-7.18 (2H, m), 7.43-7.51 (1H, m), 7.75-7.82 (1H, m), 7.83-7.89 (1H, m), 7.92-7.99 (1H, m), 8.06-8.16 (2H, m).

### Reference Example 221

### Ethyl 2-fluoro-5-[2-[3-(trifluoromethyl)benzyl]-1-benzothiophen-7-yl]benzoate

4,4,5,5-Tetramethyl-2-[2-[3-(trifluoromethyl)benzyl]-1-benzothiophen-7-yl]-1,3,2-dioxaborolane obtained in Reference Example 193 and ethyl 5-bromo-2-fluorobenzoate were used in the same manner as in Reference Example 220 to obtain the titled compound in the form of an oily substance. Yield: 83%.

¹H NMR (CDCl₃) δ: 1.39 (3H, t, J=7.2 Hz), 4.27 (2H, s), 4.41 (2H, q, J=7.2 Hz), 7.11 (1H, s), 7.19-7.31 (2H, m), 7.38-7.57 (5H, m), 7.66-7.72 (1H, m), 7.78-7.85 (1H, m), 8.22 (1H, dd, J=7.0, 2.4 Hz).

### Reference Example 222

### Ethyl 4-fluoro-3-[2-[3-(trifluoromethyl)benzyl]-1-benzothiophen-7-yl]benzoate

4,4,5,5-Tetramethyl-2-[2-[3-(trifluoromethyl)benzyl]-1-benzothiophen-7-yl]-1,3,2-dioxaborolane obtained in Reference Example 193 and ethyl 3-bromo-4-fluorobenzoate were used in the same manner as in Reference Example 220 to obtain the titled compound in the form of an oily substance. Yield: 79%.

¹H NMR (CDCl₃) δ: 1.37 (3H, t, J=7.2 Hz ), 4.26 (2H, s), 4.37 (2H, q, J=7.2 Hz), 7.10 (1H, s), 7.20-7.33 (2H, m), 7.37-7.56 (5H, m), 7.69-7.76 (1H, m), 8.06-8.14 (1H, m), 8.28 (1H, dd, J=7.2, 2.3 Hz).

### Reference Example 223

### Ethyl 2-methyl-3-[2-[3-(trifluoromethyl)benzyl]-1-benzothiophen-7-yl]benzoate

4,4,5,5-Tetramethyl-2-[2-[3-(trifluoromethyl)benzyl]-1-benzothiophen-7-yl]-1,3,2-dioxaborolane obtained in Reference Example 193 and ethyl 3-bromo-2-methylbenzoate were used in the same manner as in Reference Example 220 to obtain the titled compound in the form of an oily substance. Yield: 93%.

¹H NMR (CDCl₃) δ: 1.37-1.44 (3H, m), 2.32 (3H, s), 4.23 (2H, s), 4.33-4.43 (2H, m), 7.06-7.15 (2H, m), 7.26-7.34 (1H, m), 7.36-7.54 (6H, m), 7.65-7.71 (1H, m), 7.84-7.92 (1H, m).

### Reference Example 224

### Ethyl 5-[2-(3-chloro-5-fluorobenzyl)-1-benzothiophen-7-yl]-2-fluorobenzoate

2-[2-(3-Chloro-5-fluorobenzyl)-1-benzothiophen-7-yl]-4,4,5,5-tetramethyl-1,3,2-dioxaborolane obtained in Reference Example 192 and ethyl 5-bromo-2-fluorobenzoate were used in the same manner as in Reference Example 220 to obtain the titled compound in the form of an oily substance. Yield: 58%.

¹H NMR (CDCl₃) δ: 1.39 (3H, t, J=7.0 Hz), 4.18 (2H, s), 4.41 (2H, q, J=7.0 Hz), 6.84-6.93 (1H, m), 6.94-7.01 (1H, m), 7.06 (1H, s), 7.13 (1H, s), 7.19-7.34 (2H, m), 7.43 (1H, t; J=7.6 Hz ), 7.66-7.73 (1H, m), 7.78-7.86 (1H, m), 8.22 (1H, dd, J=6.8, 2.7 Hz).

### Reference Example 225

### Ethyl 3-fluoro-5-(4,4,5,5-tetramethyl-1,3,2-dioxaborolan-2-yl]benzoate

[1,1-Bis(diphenylphosphino)ferrocene)dichloropalladium (II) complex with dichloromethane (265 mg, 0.32 mmol) was added to a mixture of ethyl 3-bromo-5-fluorobenzoate (1.60 g, 6.48 mmol), 4,4,4′,4′,5,5,5′,5′-octamethyl-2,2′-bi-1,3,2-dioxaborolane (1.97 g, 7.78 mmol), and potassium acetate (763 mg, 7.78 mmol) in DMSO (50 mL), and the mixture was heated and stirred for 16 hours to 85° C. The reaction solution was poured into ethyl acetate, and the mixture was washed with saturated brine, dried over anhydrous magnesium sulfate, and then concentrated at reduced pressure. The residue was purified by silica gel column chromatography (hexane-ethyl acetate 100:0→70:30) to give 1.5 g of the titled compound (yield 79%) in the form of an oily substance. Melting point: 95-96° C. (ethyl acetate-hexane).

¹H-NMR (CDCl₃) δ: 1.35 (12H, s), 1.40 (3H, t, J=7.2 Hz), 4.38 (2H, q, J=7.2 Hz), 7.65 (1H, ddd, J=8.7, 3.0, 0.9 Hz), 7.78 (1H, ddd, J=9.0, 2.7, 1.5 Hz), 8.22 (1H, s).

### Reference Example 226

### Ethyl 3-fluoro-5-[2-[3-(trifluoromethyl)benzyl]-1-benzothiophen-7-yl]benzoate

Ethyl 3-fluoro-5-(4,4,5,5-tetramethyl-1,3,2-dioxaborolan-2-yl]benzoate obtained in Reference Example 225 and the 7-bromo-2-[3-(trifluoromethyl)benzyl]-1-benzothiophene obtained in Reference Example 190 were used in the same manner as in Reference Example 220 to obtain the titled compound. Yield: 51%.

¹H-NMR (CDCl₃) δ: 1.39 (3H, t, J=7.2 Hz), 4.28 (2H, s), 4.40 (2H, q, J=7.2 Hz), 7.11 (1H, s), 7.32 (1H, d, J=7.2 Hz), 7.39-7.60 (6H, m), 7.68-7.76 (2H, m), 8.15 (1H, t, J=1.5 Hz).

### Reference Example 227

### Ethyl 3-[2-(3-chloro-5-fluorobenzyl)-1-benzothiophen-7-yl]-5-fluorobenzoate

Ethyl 3-fluoro-5-(4,4,5,5-tetramethyl-1,3,2-dioxaborolan-2-yl]benzoate obtained in Reference Example 225 and the 7-bromo-2-(3-chloro-5-fluorobenzyl)-1-benzothiophene obtained in Reference Example 191 were used in the same manner as in Reference Example 220 to obtain the titled compound. Yield: 84%.

¹H-NMR (CDCl₃) δ: 1.40 (3H, t, J=7.2 Hz), 4.18 (2H, s), 4.40 (2H, q, J=7.2 Hz), 6.88 (1H, d, J=9.0 Hz), 6.97 (1H, d, J=8.4 Hz), 7.06 (1H, s), 7.13 (1H, s), 7.32 (1H, d, J=7.2 Hz), 7.44 (1H, t, J=7.5 Hz), 7.58 (1H, d, J=9.0 Hz), 7.68-7.77 (2H, m), 8.15 (1H, s).

### Reference Example 228

### Ethyl 3-(2-[[3-(trifluoromethyl)phenoxy]methyl]-1-benzothiophen-7-yl]benzoate

A 40% toluene solution of diethyl azodicarboxylate (1.4 mL, 3.07 mmol) was added while cooled on ice to a THF solution (25 mL) of ethyl 3-[2-(hydroxymethyl)-1-benzothiophen-7-yl]benzoate (800 mg, 2.56 mmol) obtained in Reference Example 168, triphenylphosphine (806 mg, 3.07 mmol), and 3-(trifluoromethyl)phenol (366 μL, 3.07 mmol). The reaction solution was stirred for 2 hours at room temperature, and the reaction solution was then concentrated at reduced pressure. The resulting residue was suspended in hexane and filtered. The filtrate was concentrated at reduced pressure, and the residue was purified by basic silica gel column chromatography (hexane:ethyl acetate=95:5→80:20) to give 843 mg of the titled compound (yield 72%).

¹H-NMR (CDCl₃) δ: 1.41 (3H, t, J=7.1 Hz), 4.42 (2H, q, J=7.1 Hz), 5.35 (2H, s) 7.16 (1H, dd, J=8.5, 2.2 Hz), 7.21-7.25 (2H, m), 7.36-7.44 (3H, m), 7.45-7.51 (1H, m), 7.58 (1H, t, J=7.8 Hz), 7.78 (1H, dd, J=7.8, 1.2 Hz), 7.89-7.94 (1H, m), 8.10 (1H, dt, J=7.8, 1.5 Hz), 8.37 (1H, t, J=1.8 Hz).

### Reference Example 229

### Ethyl 3-[2-[(3-chloro-5-fluorophenoxy)methyl]-1-benzothiophen-7-yl]benzoate

Ethyl 3-[2-(hydroxymethyl)-1-benzothiophen-7-yl]benzoate obtained in Reference Example 168 and 3-chloro-5-fluorophenol were used in the same manner as in Reference Example 228 to obtain the titled compound. Yield: 65%.

¹H-NMR (CDCl₃) δ: 1.41 (3H, t, J=7.1 Hz), 4.42 (2H, q, J=7.1 Hz), 5.29 (2H, s), 6.58-6.65 (1H, m), 6.72 (1H, dt, J=8.2, 2.1 Hz), 6.78-6.82 (1H, m), 7.37-7.43 (2H, m), 7.45-7.51 (1H, m), 7.54-7.61 (1H, m), 7.78 (1H, dd, J=7.8, 1.2 Hz), 7.91 (1H, ddd, J=7.8, 1.9, 1.1 Hz), 8.10 (1H, dt, J=7.8, 1.5 Hz), 8.37 (1H, t, J=1.6 Hz).

### Reference Example 230

### 7-Bromo-2-[[3-(trifluoromethyl)phenoxy]methyl]-1-benzothiophene

(7-Bromo-1-benzothiophen-2-yl)methanol obtained in Reference Example 162 and 3-(trifluoromethyl)phenol were used in the same manner as in Reference Example 228 to obtain the titled compound. Yield: 97%.

¹H-NMR (CDCl₃) δ: 5.36 (2H, s), 7.18 (1H, d, J=8.8 Hz), 7.21-7.30 (3H, m), 7.37-7.46 (2H, m), 7.49 (1H, t, J=7.7 Hz).

### Reference Example 231

### 4,4,5,5-tetramethyl-2-(2-[[3-(trifluoromethyl)phenoxy]methyl]-1-benzothiophen-7-yl]-1,3,2-dioxaborolane

7-Bromo-2-[[3-(trifluoromethyl)phenoxy]methyl]-1-benzothiophene obtained in Reference Example 230 was used in the same manner as in Reference Example 192 to obtain the titled compound. Yield: 61%.

¹H-NMR (CDCl₃) δ: 1.41 (12H, s), 5.36 (2H, d, J=1.1 Hz), 7.18 (1H, dd, J=8.2, 2.2 Hz), 7.21-7.29 (2H, m), 7.32-7.44 (3H, m), 7.83 (1H, ddd, J=9.8, 7.6, 1.4 Hz).

### Reference Example 232

### Ethyl 3-fluoro-5-[2-[[3-(trifluoromethyl)phenoxy]methyl]-1-benzothiophen-7-yl]benzoate

Ethyl 3-bromo-5-fluorobenzoate and 4,4,5,5-tetramethyl-2-[2-[[3-(trifluoromethyl)phenoxy]methyl]-1-benzothiophen-7-yl]-1,3,2-dioxaborolane obtained in Reference Example 231 were used in the same manner as in Reference Example 220 to obtain the titled compound. Yield: 61%.

¹H-NMR (CDCl₃) δ: 1.41 (3H, t, J=7.0 Hz), 4.41 (2H, q, J=7.1 Hz), 5.35 (2H, s), 7.12-7.20 (1H, m), 7.24 (2H, dd, J=4.1, 1.4 Hz), 7.35-7.52 (4H, m), 7.59-7.66 (1H, m), 7.73-7.84 (2H, m), 8.16-8.19 (1H, m).

### Reference Example 233

### 3-[1-Methyl-2-[[3-(trifluoromethyl)phenyl]amino]-1H-benzimidazol-4-yl]benzoic acid

Sodium hydroxide (80 mg, 1.99 mmol) and 4 to 5 drops of water were added to an ethanol (20 mL) solution of ethyl 3-[1-methyl-2-[[3-(trifluoromethyl)phenyl]amino]-1H-benzimidazol-4-yl]benzoate (1.75 g, 3.98 mmol) obtained in Reference Example 142, and the mixture was stirred for 6 hours at 45° C. The reaction mixture was stirred for 6 hours at room temperature. The reaction solution was diluted with water and neutralized with 10% citric acid aqueous solution. The resulting crystals were washed with water and hexane and dried at reduced pressure to give 1.49 g of the titled compound (yield 60%) in solid form.

¹H-NMR (DMSO-d₆) δ: 3.34 (3H, s), 7.22 (1H , t, J=8.0 Hz), 7.29 (1H, d, J=7.6 Hz), 7.39 (1H, d, J=8.0 Hz), 7.45 (1H, d, J=7.6 Hz), 7.57 (2H, t, J=7.6 Hz), 7.93 (1H, d, J=8.0 Hz), 8.30 (1H, d, J=8.4 Hz), 8.42 (1H, d, J=7.6 Hz), 8.53 (1H, br s), 8.83 (1H, br s), 9.49 (1H, br s), 1H unconfirmed.

### Reference Example 234

### 3-[1-Methyl-2-[3-(trifluoromethyl)phenoxy]-1H-benzimidazol-4-yl]benzoic acid

Ethyl 3-[1-methyl-2-[3-(trifluoromethyl)phenoxy]-1H-benzimidazol-4-yl]benzoate obtained in Reference Example 143 was used in the same manner as in Reference Example 233 to obtain the titled compound. Yield: 99%.

¹H-NMR (DMSO-d₆) δ: 3.80 (3H, s), 7.35 (1H, t, J=7.6 Hz), 7.46-7.50 (1H, m), 7.52-7.55 (2H, m), 7.66 (1H, d, J=7.6 Hz), 7.72 (1H, t, J=7.6 Hz), 7.87-7.93 (2H, m), 8.05 (1H, br s), 8.19 (1H, ddd, J=7.8, 1.6, 1.2 Hz), 8.58 (1H, dd, J=1.6, 1.6 Hz), 1H unconfirmed.

### Reference Example 235

### 3-[1-Methyl-2-[[3-(trifluoromethyl)phenyl]sulfanyl]-1H-benzimidazol-4-yl]benzoic acid

Ethyl 3-[1-methyl-2-[[3-(trifluoromethyl)phenyl]sulfanyl]-1H-benzimidazol-4-yl]benzoate obtained in Reference Example 144 was used in the same manner as in Reference Example 233 to obtain the titled compound. Yield: 89%.

¹H-NMR (DMSO-d₆) δ: 3.86 (3H, s), 7.44 (1H, t, J=8.0 Hz), 7.51-7.55 (2H, m), 7.61-7.66 (2H, m), 7.71 (2H, d, J=7.6 Hz), 7.91-7.93 (1H, m), 7.95 (1H, br s), 8.25 (1H, d, J=8.0 Hz), 8.56 (1H, br s), 13.2 (1H, br s).

### Reference Example 236

### 3-[2-(3-Chlorobenzyl)-1-benzofuran-7-yl]benzoic acid

Ethyl 3-[2-(3-chlorobenzyl)-1-benzofuran-7-yl]benzoate obtained in Reference Example 147 was used in the same manner as in Reference Example 5 to obtain the titled compound. Yield: 95%.

¹H-NMR (CDCl₃) δ: 4.07 (2H, s), 6.42 (1H, s), 7.23-7.31 (5H, m), 7.39-7.52 (3H, m), 8.06 (2H, dd, J=26.0, 6.4 Hz), 8.58 (1H, s), 1H, unconfirmed.

### Reference Example 237

### 3-[3-Methyl-2-[3-(trifluoromethyl)benzyl]-1-benzothiophen-7-yl]benzoic acid

Ethyl 3-[3-methyl-2-[3-(trifluoromethyl)benzyl]-1-benzothiophen-7-yl]benzoate obtained in Reference Example 208 was used in the same manner as in Reference Example 5 to obtain the titled compound. Yield: 70%; melting point: 172-173° C. (ethyl acetate).

¹H-NMR (DMSO-d₆) δ: 2.43 (3H, s), 4.38 (2H, s), 7.42 (1H, d, J=7.2 Hz), 7.48-7.60 (4H, m), 7.60-7.69 (2H, m), 7.78 (1H, d, J=7.6 Hz), 7.89 (1H, d, J=6.8 Hz), 7.99 (1H, d, J=7.6 Hz), 8.20 (1H, s), 13.12 (1H, br s).

### Reference Example 238

### 2-[2-[3-(Trifluoromethyl)benzyl]-1-benzothiophen-7-yl]pyridine-4-carboxylic acid

Methyl 2-[2-[3-(trifluoromethyl)benzyl]-1-benzothiophen-7-yl]pyridine-4-carboxylate obtained in Reference Example 209 was used in the same manner as in Reference Example 5 to obtain the titled compound. Yield: 67%; melting point: 231-232° C. (ethyl acetate).

¹H-NMR (DMSO-d₆) δ: 4.41 (2H, s), 7.32 (1H, s), 7.48-7.56(1H, m), 7.57-7.70 (3H, m), 7.73 (1H, s), 7.80 (1H, dd, J=5.1, 1.3 Hz), 7.92 (1H, d, J=7.2 Hz), 8.12 (1H, d, J=7.0 Hz), 8.53 (1H, s), 8.93 (1H, d, J=5.7 Hz), 13.84 (1H, br s).

### Reference Example 239

### 2-[2-(3-Chloro-5-fluorobenzyl)-1-benzothiophen-7-yl]pyridine-4-carboxylic acid

Methyl 2-[2-(3-chloro-5-fluorobenzyl)-1-benzothiophen-7-yl]pyridine-4-carboxylate obtained in Reference Example 210 was used in the same manner as in Reference Example 5 to obtain the titled compound. Yield: 76%; melting point: 205-206° C.(ethyl acetate).

¹H-NMR (DMSO-d₆) δ: 4.32 (2H, s), 7.23 (1H, d, J=9.5 Hz), 7.32 (3H, d, J=8.3 Hz), 7.53 (1H, t, J=7.8 Hz), 7.80 (1H, d, J=4.9 Hz), 7.91 (1H, d, J=8.0 Hz), 8.12 (1H, d, J=7.6 Hz), 8.53 (1H, s), 8.92 (1H, d, J=5.3 Hz), 13.92 (1H, s).

### Reference Example 240

### 2-[3-Methyl-2-[3-(trifluoromethyl)benzyl]-1-benzothiophen-7-yl]pyridine-4-carboxylic acid

Methyl 2-[3-methyl-2-[3-(trifluoromethyl)benzyl]-1-benzothiophen-7-yl]pyridine-4-carboxylate obtained in Reference Example 211 was used in the same manner as in Reference Example 5 to obtain the titled compound. Yield: 85%; melting point: 271-272° C. (ethyl acetate).

¹H-NMR (DMSO-d₆) δ: 2.42 (3H, s), 4.39 (2H, s), 7.52-7.61 (4H, m), 7.65 (1H, s), 7.79 (1H, d, J=4.9 Hz), 7.87 (1H, d, J=7.2 Hz), 8.14 (1H, d, J=6.8 Hz), 8.51 (1H, s), 8.90 (1H, d, J=4.9 Hz), 13.95 (1H, br s).

### Reference Example 241

### 4-[2-[3-(Trifluoromethyl)benzyl]-1-benzothiophen-7-yl]benzoic acid

Ethyl 4-[2-[3-(trifluoromethyl)benzyl]-1-benzothiophen-7-yl]benzoate obtained in Reference Example 212 was used in the same manner as in Reference Example 5 to obtain the titled compound. Yield: 93%; melting point: 200-201° C. (ethyl acetate).

¹H-NMR (DMSO-d₆) δ: 4.39 (2H, s), 7.38 (1H, s), 7.39-7.44 (1H, m), 7.46-7.52 (1H, m), 7.53-7.68 (3H, m), 7.71 (1H, s), 7.79 (2H, d, J=8.3 Hz), 7.84 (1H, dd, J=7.9, 1.1 Hz), 8.07 (2H, d, J=8.7 Hz), 13.06 (1H, s).

### Reference Example 242

### 6-[2-[3-(Trifluoromethyl)benzyl]-1-benzothiophen-7-yl]pyridine-3-carboxylic acid

Ethyl 6-[2-[3-(trifluoromethyl)benzyl]-1-benzothiophen-7-yl]pyridine-3-carboxylate obtained in Reference Example 213 was used in the same manner as in Reference Example 5 to obtain the titled compound. Yield: 71%.

¹H-NMR (DMSO-d₆) δ: 4.41 (2H, s), 7.31 (1H, s), 7.53 (1H, t, J=7.6 Hz), 7.57-7.70 (3H, m), 7.73 (1H, s), 7.93 (1H, d, J=7.2 Hz), 8.13 (1H, d, J=7.2 Hz), 8.26-8.43 (2H, m), 9.19 (1H, s), 13.57 (1H, br s).

### Reference Example 243

### 3-[4-Fluoro-2-[3-(trifluoromethyl)benzyl]-1-benzothiophen-7-yl]benzoic acid

1 N sodium hydroxide aqueous solution (1 mL, 1 mmol) was added to a THF (8 mL) and methanol (2 mL) solution of ethyl 3-[4-fluoro-2-[3-(trifluoromethyl)benzyl]-1-benzothiophen-7-yl]benzoate (200 mg, 0.44 mmol) synthesized in Reference Example 214, and the mixture was stirred for 3 hours at room temperature. The reaction solution was made acidic using 1 N hydrochloric acid aqueous solution and was extracted with ethyl acetate. The organic layer was washed with saturated brine and was then dried over anhydrous sodium sulfate. The solvent was distilled off at reduced pressure to give 118 mg of the titled compound (yield 62%).

¹H-NMR (CDCl₃) δ: 4.29 (2H, s), 7.12 (1H, dd, J=9.6, 8.0 Hz), 7.23 (1H, d, J=0.8 Hz), 7.27-7.32 (1H, m), 7.39-7.64 (5H, m), 7.86-7.92 (1H, m), 8.10-8.17 (1H, m), 8.36 (1H, t, J=1.8 Hz), 1H, unconfirmed.

### Reference Example 244

### 3-[2-(3-Chloro-5-fluorobenzyl)-4-fluoro-1-benzothiophen-7-yl]benzoic acid

Ethyl 3-[2-(3-chloro-5-fluorobenzyl)-4-fluoro-1-benzothiophen-7-yl]benzoate obtained in Reference Example 215 was used in the same manner as in Reference Example 243 to obtain the titled compound. Yield: 76%; melting point: 185-186° C. (ethyl acetate-hexane).

¹H-NMR (CDCl₃) δ: 4.18 (2H, s), 6.88 (1H, d, J=9.6 Hz), 6.97 (1H, d, J=8.4 Hz), 7.06 (1H, s), 7.11 (1H, t, J=8.4 Hz), 7.22-7.32 (2H, m), 7.58 (1H, t, J=8.1 Hz), 7.88 (1H, d, J=7.5 Hz), 8.13 (1H, d, J=8.1 Hz), 8.36 (1H, s), 1H unconfirmed.

### Reference Example 245

### 3-[2-(3,4-difluorobenzyl)-4-fluoro-1-benzothiophen-7-yl]benzoic acid

Ethyl 3-[2-(3,4-difluorobenzyl)-4-fluoro-1-benzothiophen-7-yl]benzoate obtained in Reference Example 216 was used in the same manner as in Reference Example 243 to obtain the titled compound. Yield: 75%; melting point: 224-225° C. (ethyl acetate-hexane).

¹H-NMR (CDCl₃) δ: 4.18 (2H, s), 6.92-7.18 (4H, m), 7.20-7.31 (2H, m), 7.57 (1H, t, J=7.5 Hz), 7.87 (1H, d, J=7.8 Hz), 8.12 (1H, d, J=7.5 Hz), 8.34 (1H, s), 1H unconfirmed.

### Reference Example 246

### 3-[4-Chloro-2-(3,4-difluorobenzyl)-1-benzothiophen-7-yl]benzoic acid

Ethyl 3-[4-chloro-2-(3,4-difluorobenzyl)-1-benzothiophen-7-yl]benzoate obtained in Reference Example 217 was used in the same manner as in Reference Example 243 to obtain the titled compound. Yield: 82%; melting point: 214-215° C. (ethyl acetate-hexane).

¹H-NMR (CDCl₃) δ: 4.19 (2H, s), 6.90-7.18 (3H, m), 7.20-7.28 (2H, m), 7.43 (1H, d, J=8.1 Hz), 7.58 (1H, t, J=7.5 Hz), 7.87 (1H, d, J=7.5 Hz), 8.14 (1H, d, J=7.8 Hz), 8.35 (1H, s), 1H unconfirmed.

### Reference Example 247

### 3-[4-Chloro-2-(3-chloro-5-fluorobenzyl)-1-benzothiophen-7-yl]benzoic acid

Ethyl 3-[4-chloro-2-(3-chloro-5-fluorobenzyl)-1-benzothiophen-7-yl]benzoate obtained in Reference Example 218 was used in the same manner as in Reference Example 243 to obtain the titled compound. Yield: 76%; melting point: 110-114° C. (ethyl acetate-hexane).

¹H-NMR (CDCl₃) δ: 4.20 (2H, s), 6.89 (1H, d, J=8.4 Hz), 6.93-7.00 (1H, m), 7.06 (1H, s), 7.24-7.58 (4H, m), 7.75-7.83 (1H, m), 8.04-8.10 (1H, m), 8.31 (1H, s), 1H unconfirmed.

### Reference Example 248

### 3-Fluoro-5-[2-[3-(trifluoromethyl)benzyl]-1-benzothiophen-7-yl]benzoic acid

Ethyl 3-fluoro-5-[2-[3-(trifluoromethyl)benzyl]-1-benzothiophen-7-yl]benzoate obtained in Reference Example 226 was used in the same manner as in Reference Example 243 to obtain the titled compound. Yield: 85%; melting point: 140-142° C. (ethyl acetate-hexane).

¹H-NMR (CDCl₃) δ: 4.28 (2H, s), 7.11 (1H, s), 7.32 (1H, d, J=7.5 Hz), 7.40-7.56 (5H, m), 7.62-7.66 (1H, m), 7.71 (1H, d, J=6.6 Hz), 7.76-7.83 (1H, m), 8.22 (1H, s), 1H, unconfirmed.

### Reference Example 249

### 3-[2-(3-Chloro-5-fluorobenzyl)-1-benzothiophen-7-yl]-5-fluorobenzoic acid

Ethyl 3-[2-(3-chloro-5-fluorobenzyl)-1-benzothiophen-7-yl]-5-fluorobenzoate obtained in Reference Example 227 was used in the same manner as in Reference Example 243 to obtain the titled compound. Yield: 82%; melting point: 168-170° C. (ethyl acetate-hexane).

¹H-NMR (CDCl₃) δ: 4.19 (2H, s), 6.88 (1H, d, J=8.4 Hz), 6.96 (1H, d, J=8.4 Hz), 7.06 (1H, s), 7.13 (1H, s), 7.34 (1H, d, J=7.5 Hz), 7.44 (1H, t, J=7.5 Hz), 7.65 (1H, d, J=9.0 Hz), 7.72 (1H, d, J=7.5 Hz), 7.81 (1H, d, J=8.7 Hz), 8.23 (1H, s), 1H, unconfirmed.

### Reference Example 250

### 3-[2-(3,4-difluorobenzyl)-1-benzothiophen-4-yl]benzoic acid

Ethyl 3-[2-(3,4-difluorobenzyl)-1-benzothiophen-4-yl]benzoate obtained in Reference Example 207 was used in the same manner as in Reference Example 5 to obtain the titled compound. Yield: 86%.

¹H-NMR (CDCl₃) δ: 4.18 (2H, s), 6.93-7.02 (1H, m), 7.02-7.11 (2H, m), 7.14 (1H, s), 7.31-7.40 (2H, m), 7.60 (1H, t, J=7.7 Hz), 7.74-7.83 (2H, m), 8.15 (1H, d, J=7.7 Hz), 8.31 (1H, s)

### Reference Example 251

### 5-[2-(3-Chloro-5-fluorobenzyl)-1-benzothiophen-7-yl]pyridine-3-carboxylic acid

1 N sodium hydroxide aqueous solution (1.4 ml) was added at room temperature to a THF (6 mL)-methanol (3 mL) mixed solution of ethyl 5-[2-(3-chloro-5-fluorobenzyl)-1-benzothiophen-7-yl]pyridine-3-carboxylate (410 mg, 0.963 mmol) obtained in Reference Example 219, and the mixture was stirred for 5 hours. Water was poured into the reaction solution, the pH was adjusted to between 2 and 3 with 1 N hydrochloric acid aqueous solution, and the mixture was extracted with ethyl acetate. The extract was washed with water and dried over anhydrous magnesium sulfate, and the solvent was distilled off at reduced pressure. Diethyl ether was added to the residue, and 304 mg of the titled compound (yield 80%) was filtered off in solid form.

¹H NMR (DMSO-d₆) δ: 4.32 (2H, s), 7.12-7.36 (3H, m), 7.41 (1H, s), 7.45-7.61 (2H, m), 7.80-7.95 (1H, m), 8.52 (1H, t, J=2.3 Hz), 9.08 (1H, d, J=2.6 Hz), 9.13 (1H, d, J=2.3 Hz), 13.62 (1H, br s).

### Reference Example 252

### 6-[2-(3-Chloro-5-fluorobenzyl)-1-benzothiophen-7-yl]pyridine-2-carboxylic acid

Methyl 6-[2-(3-chloro-5-fluorobenzyl)-1-benzothiophen-7-yl]pyridine-2-carboxylate obtained in Reference Example 220 was used in the same manner as in Reference Example 251 to obtain the titled compound in solid form. Yield: 74%.

¹H NMR (DMSO-d₆) δ: 4.33 (2H, s), 7.13-7.35 (4H, m), 7.52 (1H, t, J=7.6 Hz), 7.91 (1H, d, J=7.9 Hz), 8.02 (1H, d, J=7.6 Hz), 8.06-8.17 (2H, m), 8.42 (1H, d, J=7.9 Hz), 13.27 (1H, br s).

### Reference Example 253

### 2-Fluoro-5-[2-[3-(trifluoromethyl)benzyl]-1-benzothiophen-7-yl]benzoic acid

Ethyl 2-fluoro-5-[2-[3-(trifluoromethyl)benzyl]-1-benzothiophen-7-yl]benzoate obtained in Reference Example 221 was used in the same manner as in Reference Example 251 to obtain the titled compound in solid form. Yield: 73%.

¹H NMR (DMSO-d₆) δ: 4.39 (2H, s), 7.33-7.41 (2H, m), 7.42-7.68 (5H, m), 7.71 (1H, s), 7.79-7.85 (1H, m), 7.87-7.95 (1H, m), 8.12 (1H, dd, J=7.0, 2.5 Hz), 13.42 (1H, br s).

### Reference Example 254

### 4-Fluoro-3-[2-[3-(trifluoromethyl)benzyl]-1-benzothiophen-7-yl]benzoic acid

Ethyl 4-fluoro-3-[2-[3-(trifluoromethyl)benzyl]-1-benzothiophen-7-yl]benzoate obtained in Reference Example 222 was used in the same manner as in Reference Example 251 to obtain the titled compound in solid form. Yield: 82%.

¹H NMR (DMSO-d₆) δ: 4.38 (2H, s), 7.31-7.39 (2H, m), 7.44-7.67 (5H, m), 7.71 (1H, s), 7.83-7.90 (1H, m), 8.03-8.14 (2H, m), 13.19 (1H, s).

### Reference Example 255

### 2-Methyl-3-[2-[3-(trifluoromethyl)benzyl]-1-benzothiophen-7-yl]benzoic acid

Ethyl 2-methyl-3-[2-[3-(trifluoromethyl)benzyl]-1-benzothiophen-7-yl]benzoate obtained in Reference Example 223 was used in the same manner as in Reference Example 251 to obtain the titled compound in solid form. Yield: 82%.

¹H NMR (DMSO-d₆) δ: 2.20 (3H, s), 4.34 (2H, s), 7.15 (1H, d, J=6.4 Hz), 7.31-7.48 (4H, m), 7.51-7.66 (3H, m), 7.69 (1H, s), 7.77-7.86 (2H, m), 12.99 (1H, br s).

### Reference Example 256

### 5-[2-(3-Chloro-5-fluorobenzyl)-1-benzothiophen-7-yl]-2-fluorobenzoic acid

Ethyl 5-[2-(3-chloro-5-fluorobenzyl)-1-benzothiophen-7-yl]-2-fluorobenzoate obtained in Reference Example 224 was used in the same manner as in Reference Example 251 to obtain the titled compound in solid form. Yield: 77%.

¹H NMR (DMSO-d₆) δ: 4.31 (2H, s), 7.16-7.25 (1H, m), 7.27-7.34 (2H, m), 7.35-7.42 (2H, m), 7.43-7.53 (2H, m), 7.83 (1H, d, J=6.8 Hz), 7.87-7.96 (1H, m), 8.12 (1H, dd, J=6.8, 2.4 Hz), 13.42 (1H, br s).

### Reference Example 257

### Ethyl 3-[2-[3-fluoro-5-(methylsulfonyl)benzyl]-1-benzothiophen-7-yl]benzoate

A mixture of 7-chloro-2-[3-fluoro-5-(methylsulfonyl)benzyl]-1-benzothiophene (0.7 g, 1.97 mmol) obtained in Reference Example 196, [3-(ethoxycarbonyl)phenyl]boronic acid (0.46 g, 2.37 mmol), palladium acetate (13.3 mg, 0.059 mmol), 2-dicyclohexylphosphino-2′,4′,6′-triisopropyl biphenyl (56.4 mg, 0.12 mmol), potassium phosphate (0.84 g, 3.95 mmol), and THF (15 mL) was heated to reflux for 24 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=3:2) to give 0.70 g of the titled compound (yield 76%) in the form of an oily substance.

¹H NMR (CDCl₃) δ: 1.39 (3H, t, J=7.2 Hz), 3.05 (3H, s), 4.30 (2H, s), 4.40 (2H, q, J=7.2 Hz), 7.17 (1H, s), 7.20-7.35 (1H, m), 7.35 (1H, d, J=7.2 Hz), 7.45 (1H, t, J=7.8 Hz), 7.56 (1H, d, J=7.8 Hz), 7.50-7.60 (1H, m), 7.66 (1H, s), 7.71 (1H, d, J=7.8 Hz), 7.86 (1H, d, J=7.5 Hz), 8.07 (1H, d, J=8.1 Hz), 8.33 (1H, s).

### Reference Example 258

### Ethyl 3-[2-[(5-methoxypyridin-3-yl)methyl]-1-benzothiophen-7-yl]benzoate

Ethyl 3-[2-(bromomethyl)-1-benzothiophen-7-yl]benzoate (1.33 g, 3.54 mmol) obtained in Reference Example 179 and 3-methoxypyridine-5-boronic acid pinacol ester (1.00 g, 4.25 mmol) were used in the same manner as in Reference Example 4 to obtain the titled compound. 24% yield, oily substance.

¹H-NMR (DMSO-d₆) δ: 1.40 (3H, t, J=7.2 Hz), 3.82 (3H, s), 4.21 (2H, s), 4.40 (2H, q, J=7.2 Hz), 7.06-7.13 (2H, m), 7.33 (1H, dd, J=1.2, 7.5 Hz), 7.44 (1H, t, J=7.8 Hz), 7.55 (1H, t, J=7.5 Hz), 7.69 (1H, dd, J=1.2, 7.8 Hz), 7.84-7.91 (1H, m), 8.05-8.11 (1H, m), 8.17-8.23 (2H, m), 8.32-8.36 (1H, m).

### Reference Example 259

### 3-[2-[3-(Trifluoromethyl)benzyl]-1,3-benzothiazol-4-yl]benzoic acid

4-Bromo-2-[3-(trifluoromethyl)benzyl]-1,3-benzothiazole obtained in Reference Example 150 was used in the same manner as in Reference Example 10 to obtain the titled compound. Yield: 33%; melting point: 152-153° C. (ethyl acetate-hexane).

¹H-NMR (CDCl₃) δ: 4.50 (2H, s), 7.41-7.68 (6H, m), 7.82 (1H, d, J=8.1 Hz), 8.10-8.16 (2H, m), 8.59 (1H, s), 8.31 (1H, t, J=1.8 Hz), 1H, unconfirmed.

### Working Example 1

### N-(2-Cyanoethyl)-3-[2-[3-(trifluoromethyl)benzyl]-1,2,3,4-tetrahydroisoquinolin-8-yl]benzamide

1 N sodium hydroxide aqueous solution (1.57 mL, 1.57 mmol) was added to an ethanol (5 mL) solution of Ethyl 3-[2-[3-(trifluoromethyl)benzyl]-1,2,3,4-tetrahydroisoquinolin-8-yl]benzoate (0.23 g, 0.52 mmol) obtained in Reference Example 2, and the mixture was stirred for 4 hours. The reaction solution was neutralized with the addition of 1 N hydrochloric acid (1.57 mL), and the solvent was distilled off at reduced pressure. A mixture of the residue, 3-aminopropanenitrile (0.046 mL, 0.63 mmol), WSC (135 mg, 0.79 mmol), HOBt (106 mg, 0.79 mmol), and DMF (5 mL) was stirred 15 hours. The reaction solution was diluted with saturated aqueous sodium bicarbonate and extracted with ethyl acetate. The extract was washed with water and dried over anhydrous magnesium sulfate, and the solvent was then distilled off at reduced pressure. The residue was purified by silica gel column chromatography (hexane-ethyl acetate=1:1), and the resulting oily substance was crystallized from hexane-diethyl ether to give 0.18 g of the titled compound (yield 74%). Melting point: 114-117° C.

¹H-NMR (CDCl₃) δ: 2.69 (2H, t, J=6.0 Hz), 2.76 (2H, t, J=6.0 Hz), 2.96 (2H, J=5.6 Hz), 3.50 (2H, s), 3.62 (2H, s), 3.74 ((2H, q, J=6.3 Hz), 6.57 (1H, m), 7.02 (1H, d, J=6.6 Hz), 7.15 (1H, t, J=7.5 Hz), 7.21 (1H, d, J=7.5 Hz), 7.35-7.55 (5H, m), 7.55 (1H, s), 7.67 (1H, s), 7.70-7.80 (1H, m).

### Working Example 2

### N-(2-Hydroxyethyl)-3-[2-[3-(trifluoromethyl)benzyl]-1,2,3,4-tetrahydroisoquinolin-8-yl]benzamide hydrochloride

1 N sodium hydroxide aqueous solution (1.64 mL, 1.64 mmol) was added to an ethanol (5 mL) solution of ethyl 3-[2-[3-(trifluoromethyl)benzyl]-1,2,3,4-tetrahydroisoquinolin-8-yl]benzoate (0.24 g, 0.55 mmol) obtained in Reference Example 2, and the mixture was stirred for 5 hours. The reaction solution was neutralized with the addition of 1 N hydrochloric acid (1.64 mL), and the solvent was distilled off at reduced pressure. A mixture of the residue, 2-aminoethanol (0.039 mL, 0.66 mmol), WSC (141 mg, 0.81 mmol), HOBt (111 mg, 0.81 mmol), and DMF (5 mL) was stirred 15 hours. The reaction solution was diluted with saturated aqueous sodium bicarbonate and extracted with ethyl acetate. The extract was washed with water and dried over anhydrous magnesium sulfate, and the solvent was then distilled off at reduced pressure. The residue was purified by silica gel column chromatography (hexane-ethyl acetate=1:1), the resulting oily substance was dissolved in ethyl acetate (5 mL), and 4 N hydrogen chloride/ethyl acetate (0.5 mL) was added. The solvent was distilled off at reduced pressure to give 0.19 g of the titled compound (yield 71%) in the form of amorphous solids.

¹H-NMR (DMSO-d₆) δ: 3.37 (2H, t, J=6.9 Hz), 3.53 (2H, t, J=6.9 Hz), 3.30-3.70 (4H, m), 4.00-4.10 (1H, m), 4.40-4.60 (3H, m), 7.21 (1H, d, J=7.2 Hz), 7.31 (1H, d, J=7.5 Hz), 7.37-7.45 (2H, m), 7.51 (1H, t, J=7.8 Hz), 7.61 (1H, t, J=7.2 Hz), 7.76 (1H, d, J=8.7 Hz), 7.81 (2H, s), 7.85-7.95 (2H, m), 8.55-8.65 (1H, m), 10.79 (1H, br s).

### Working Example 3

### N-(2-Cyanoethyl)-3-[2-[3-(trifluoromethyl)benzyl]-1,2,3,4-tetrahydroisoquinolin-5-yl]benzamide

3-Aminopropanenitrile (43.5 μL, 0.59 mmol) was added to a DMF (2 mL) solution of 3-[2-[3-(trifluoromethyl)benzyl]-1,2,3,4-tetrahydroisoquinolin-5-yl]benzoic acid (200 mg, 0.49 mmol) obtained in Reference Example 5, WSC (113 mg, 0.59 mmol), and HOBt (79.7 mg, 0.59 mmol), and the mixture was stirred for 5 hours at room temperature. The addition of saturated sodium bicarbonate aqueous solution to the reaction solution was followed by extraction with ethyl acetate. The organic layer was washed with water, dried over anhydrous magnesium sulfate, and then concentrated at reduced pressure. The residue was purified by silica gel column chromatography to give 60 mg of the titled compound (yield 26%) in the form of amorphous solids.

¹H-NMR (CDCl₃) δ: 2.62-2.80 (6H, m), 3.65-3.78 (6H, m), 6.65 (1H, br s), 7.06 (2H, t, J=7.6 Hz), 7.21 (1H, t, J=7.5 Hz), 7.41-7.57 (4H, m), 7.61 (1H, d, J=7.9 Hz), 7.66 (1H, s), 7.69-7.82 (2H, m).

### Working Example 4

### N-(2-Cyanoethyl)-3-[4-methyl-2-[3-(trifluoromethyl)benzyl]-1,2,3,4-tetrahydroisoquinolin-8-yl]benzamide hydrochloride

Ethyl 3-[4-methyl-2-[3-(trifluoromethyl)benzyl]-1,2,3,4-tetrahydroisoquinolin-8-yl]benzoate obtained in Reference Example 7 and 3-aminopropanenitrile were used in the same manner as in Working Example 2 to obtain the titled compound. Yield 65%, amorphous solids.

¹H-NMRR (DMSO-d₆) δ: 1.36 (3H, d, J=6.9 Hz), 2.81 (2H, t, J=6.3 Hz), 3.00-3.15 (1H, m), 3.20-3.70 (4H, m), 3.90-4.75 (4H, m), 7.23 (1H, d, J=6.9 Hz), 7.40-7.55 (3H, m), 7.54 (1H, d, J=7.5 Hz), 7.62 (1H, t, J=7.8 Hz), 7.77 (1H, d, J=7.8 Hz), 7.83 (2H, s), 7.92 (2H, s), 9.05 (1H, br s), 10.77 (1H, br s).

### Working Example 5

### N-(2-Hydroxyethyl)-3-[4-methyl-2-[3-(trifluoromethyl)benzyl]-1,2,3,4-tetrahydroisoquinolin-8-yl]benzamide hydrochloride

Ethyl 3-[4-methyl-2-[3-(trifluoromethyl)benzyl]-1,2,3,4-tetrahydroisoquinolin-8-yl]benzoate obtained in Reference Example 7 and 2-aminoethanol were used in the same manner as in Working Example 2 to obtain the titled compound. Yield 61 %, amorphous solids.

¹H-NMR (DMSO-d₆) δ: 1.30-1.50 (3H, m), 3.00-3.15 (1H, m), 3.30-3.70 (5H, m), 3.90-4.70 (6H, m), 7.20-7.30 (1H, m), 7.40-7.70 (5H, m), 7.75-7.90 (3H, m), 7.91 (2H, s), 8.55-8.65 (1H, m), 10.62 (1H, br s).

### Working Example 6

### N-(2-Cyanoethyl)-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10 and 3-aminopropanenitrile were used in the same manner as in Working Example 3 to obtain the titled compound. Yield: 65%; melting point: 107-108° C. (ethyl acetate-hexane).

¹H-NMR (CDCl₃) δ: 2.77 (2H, t, J=6.0 Hz), 3.74 (2H, q, J=6.0 Hz), 4.17 (2H, s), 6.60 (1H, s), 6.64 (1H, br s), 7.24-7.38 (2H, m), 7.39-7.58 (6H, m), 7.72-7.77 (2H, m), 8.01 (1H, s).

### Working Example 7

### N-(2-Hydroxyethyl)-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10 and 2-aminoethanol were used in the same manner as in Working Example 3 to obtain the titled compound. Yield: 63%; melting point: 135-136° C. (ethyl acetate-hexane).

¹H-NMR (CDCl₃) δ: 2.43 (1H, t, J=4.8 Hz), 3.66 (2H, q, J=4.8 Hz), 3.86 (2H, q, J=4.8 Hz), 4.18 (2H, s), 6.60 (1H, s), 6.64 (1H, br s), 7.26-7.32 (2H, m), 7.38-7.60 (6H, m), 7.68-7.81 (2H, m), 8.01 (1H, s).

### Working Example 8

### N-(6-Fluoropyridin-3-yl)-3-[2-[3-(trifluoromethyl)benzyl]-1-benzofuran-4-yl]benzamide

A DMF (10 mL) solution of 3-[2-[3-(trifluoromethyl)benzyl]-1-benzofuran-4-yl]benzoic acid (400 mg, 1.0 mmol) obtained in Reference Example 10, 6-fluoropyridin-3-amine (123 mg, 1.10 mmol), HATU (418 mg, 1.10 mmol), and N,N-diisopropylethylamine (0.21 mL, 1.20 mmol) was stirred for 16 hours at 80° C. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (hexane-ethyl acetate 95:5→50:50) and was recrystallized from hexane and ethyl acetate to give 220 mg of the titled compound (yield 45%). Melting point: 117-118° C. (ethyl acetate-hexane).

¹H-NMR (CDCl₃) δ: 4.18 (2H, s), 6.60 (1H, s), 6.98 (1H, dd, J=8.4, 3.3 Hz), 7.29-7.36 (2H, m), 7.39-7.63 (6H, m), 7.77-7.87 (2H, m), 7.90 (1H, s), 8.08 (1H, s), 8.29-8.40 (2H, m).

### Working Example 9

### 3-[2-[3-(Trifluoromethyl)benzyl]-1-benzofuran-4-yl]benzamide

A mixture of 4-bromo-2-[3-(trifluoromethyl)benzyl]-1-benzofuran (293 mg, 0.825 mmol) obtained in Reference Example 9, (3-carbamoylphenyl)boronic acid (163 mg, 0.99 mmol), and tetrakis(triphenylphosphine)palladium (0) (48 mg, 0.041 mmol) in 2 N sodium carbonate aqueous solution (10 mL)-1,2-dimethoxyethane (10 mL) was reacted for 16 hours at 90° C. in a nitrogen atmosphere. The addition of water to the reaction solution was followed by extraction with ethyl acetate. The organic layer was washed with saturated brine, then dried over anhydrous sodium sulfate, and then concentrated at reduced pressure, and the residue was purified by silica gel column chromatography (ethyl acetate-hexane 5:95→60:40) to give 200 mg of the titled compound (yield 61%). Melting point: 159-160° C. (ethyl acetate-hexane).

¹H-NMR (CDCl₃) δ: 4.18 (2H, s), 5.78 (1H, br s), 6.15 (1H, br s), 6.60 (1H, s), 7.26-7.35 (2H, m), 7.38-7.58 (6H, m), 7.72-7.82 (2H, m), 8.04 (1H, t, J=1.8 Hz).

### Working Example 10

### N-(Tetrahydro-2H-pyran-4-yl)-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10 and tetrahydro-2H-pyran-4-amine were used in the same manner as in Working Example 3 to obtain the titled compound. Yield: 58%; melting point: 107-108° C. (ethyl acetate-hexane).

¹H-NMR (CDCl₃) δ: 1.49-1.65 (2H, m), 1.98-2.07 (2H, m), 3.54 (2H, dt, J=11.7, 2.1 Hz), 3.95-4.05 (2H, m), 4.16-4.35 (3H, m), 5.99 (1H, d, J=8.4 Hz), 6.59 (1H, d, J=0.9 Hz), 7.27-7.35 (2H, m), 7.38-7.56 (6H, m), 7.68-7.75 (2H, m), 7.97 (1H, t, J=1.8 Hz).

### Working Example 11

### N-Cyclopropyl-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10 and cyclopropanamine were used in the same manner as in Working Example 3 to obtain the titled compound. Yield: 87%; melting point: 120-121° C. (ethyl acetate-hexane).

¹H-NMR (CDCl₃) δ: 0.60-0.66 (2H, m), 0.85-0.93 (2H, m), 2.88-3.00 (1H, m), 4.17 (2H, s), 6.24 (1H, br s), 6.59 (1H, s), 7.25-7.34 (2H, m), 7.39-7.58 (6H, m), 7.68-7.73 (2H, m), 7.95 (1H, t, J=1.5 Hz).

### Working Example 12

### N-(2-Amino-2-oxoethyl)-3-[2-[3-(trifluoromethyl)benzyl]-1-benzofuran-4-yl]benzamide

Glycinamide hydrochloride (167 mg, 1.51 mmol) and N,N-diisopropylethylamine (0.26 mL, 1.51 mmol) were added to a DMF (10 mL) solution of 3-[2-[3-(trifluoromethyl)benzyl]-1-benzofuran-4-yl]benzoic acid (500 mg, 1.26 mmol) obtained in Reference Example 10, WSC (266 mg, 1.39 mmol), and HOBt (188 mg, 1.39 mmol), and the mixture was stirred for 5 hours at room temperature. The addition of saturated sodium bicarbonate aqueous solution to the reaction solution was followed by extraction with ethyl acetate. The organic layer was washed with water, dried over anhydrous magnesium sulfate, and then concentrated at reduced pressure. The residue was purified by basic silica gel column chromatography (ethyl acetate-methanol 99:1→80:20) to give 380 mg of the titled compound (yield 67%). Yield: 67%; melting point: 105-106° C. (ethyl acetate-hexane).

¹H-NMR (CDCl₃) δ: 4.16 (2H, s), 4.20 (2H, d, J=4.8 Hz), 5.60 (1H, br s), 6.30 (1H, br s), 6.59 (1H, s), 7.07-7.34 (3H, m), 7.38-7.61 (6H, m), 7.73 (1H, d, J=7.8 Hz), 7.79 (1H, d, J=7.5 Hz), 8.05 (1H, s).

### Working Example 13

### N-(3-Amino-3-oxopropyl)-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10 and β-alaninamide hydrochloride were used in the same manner as in Working Example 12 to obtain the titled compound. Yield: 68%; melting point: 155-156° C. (ethyl acetate-hexane).

¹H-NMR (CDCl₃) δ: 2.59 (2H, t, J=5.4 Hz), 3.76 (2H, q, J=5.4 Hz), 4.17 (2H, s), 5.38 (1H, br s), 5.69 (1H, br s), 6.62 (1H, s), 7.15 (1H, br s), 7.23-7.34 (2H, m), 7.38-7.56 (6H, m), 7.68-7.75 (2H, m), 8.01 (1H, s).

### Working Example 14

### N-(2-Methoxyethyl)-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10 and methoxyethanamine were used in the same manner as in Working Example 3 to obtain the titled compound. Yield: 63%; melting point: 91-92° C. (ethyl acetate-hexane).

¹H-NMR (CDCl₃) δ: 3.38 (3H, s), 3.57 (2H, t, J=5.1 Hz), 3.68 (2H, q, J=5.1 Hz), 4.17 2H, s), 6.55 (1H, br s), 6.61 (1H, s), 7.26-7.35 (2H, m), 7.39-7.55 (6H, m), 7.68-7.77 (2H, m), 8.01 (1H, s).

### Working Example 15

### N-1,3-thiazol-2-yl)-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10 and 2-aminothiazole were used in the same manner as in Working Example 8 to obtain the titled compound. Yield: 63%; melting point: 146-147° C. (ethyl acetate-hexane).

¹H-NMR (CDCl₃) δ: 4.17 (2H, s), 6.62 (1H, s), 6.90 (1H, d, J=3.6 Hz), 7.17 (1H, d, J=3.6 Hz), 7.26-7.35 (2H, m), 7.40-7.65 (6H, m), 7.84 (1H, d, J=8.1 Hz), 7.95 (1H, d, J=7.8 Hz), 8.21 (1H, s), 11.4 (1H, br s).

### Working Example 16

### N-Pyridin-3-yl-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10 and 3-aminopyridine were used in the same manner as in Working Example 8 to obtain the titled compound. Yield: 62%; melting point: 127-128° C. (ethyl acetate-hexane).

¹H-NMR (CDCl₃) δ: 4.18(2H, s), 6.61 (1H, s), 7.28-7.36 (3H, m), 7.41-7.63 (6H, m), 7.79 (1H, d, J=7.5 Hz), 7.85 (1H, d, J=7.8 Hz), 7.90 (1H, s), 8.10 (1H, s), 8.30 (1H, d, J=8.4 Hz), 8.40 (1H, d, J=4.5 Hz), 8.67 (1H, d, J=2.4 Hz).

### Working Example 17

### N-(2-Cyanoethyl)-3-[3-methyl-2-[3-(trifluoromethyl)benzyl]-1-benzofuran-4-yl]benzamide

3-[3-Methyl-2-[3-(trifluoromethyl)benzyl]-1-benzofuran-4-yl]benzoic acid obtained in Reference Example 15 and 3-aminopropanenitrile were used in the same manner as in Working Example 3 to obtain the titled compound. Yield: 57%; melting point: 108-109° C. (ethyl acetate-hexane).

¹H-NMR (CDCl₃) δ: 1.80 (3H, s), 2.75 (2H, t, J=6.3 Hz), 3.71 (2H, t, J=6.3 Hz), 4.11 (2H, s), 6.70 (1H, br s), 7.07 (1H, d, J=7.2 Hz), 7.21-7.28 (1H, m), 7.37-7.60 (7H, m), 7.77-7.83 (2H, m).

### Working Example 18

### N-(2-Hydroxyethyl)-3-[3-methyl-2-[3-(trifluoromethyl)benzyl]-1-benzofuran-4-yl]benzamide

3-[3-Methyl-2-[3-(trifluoromethyl)benzyl]-1-benzofuran-4-yl]benzoic acid obtained in Reference Example 15 and 2-aminoethanol were used in the same manner as in Working Example 3 to obtain the titled compound. Yield: 54%; melting point: 139-141° C. (ethyl acetate-hexane).

¹H-NMR (CDCl₃) δ: 1.79 (3H, s), 2.40 (1H, br s), 3.65 (2H, q, J=5.4 Hz), 3.85 (2H, t, J=5.4 Hz), 4.11 (2H, s), 6.63 (1H, br s), 7.08 (1H, d, J=7.5 Hz), 7.21-7.28 (1H, m), 7.37-7.60 (7H, m), 7.79-7.83 (2H, m).

### Working Example 19

### N-(2-Cyanoethyl)-3-[7-methoxy-2-[3-(trifluoromethyl)benzyl]-1-benzofuran-4-yl]benzamide

3-[7-Methoxy-2-[3-(trifluoromethyl)benzyl]-1-benzofuran-4-yl]benzoic acid obtained in Reference Example 18 and 3-aminopropanenitrile were used in the same manner as in Working Example 3 to obtain the titled compound. Yield: 74%; melting point: 126-127° C. (ethyl acetate-hexane).

¹H-NMR (CDCl₃) δ: 2.76 (2H, t, J=6.3 Hz), 3.73 (2H, q, J=6.3 Hz), 4.03 (3H, s), 4.20 (2H, s), 6.57 (1H, s), 6.63 (1H, br s), 6.85 (1H, d, J=8.1 Hz), 7.22 (1H, d, J=8.1 Hz), 7.38-7.57 (5H, m), 7.70 (2H, dd, J=7.5, 1.8 Hz), 7.96 (1H, d, J=1.8 Hz).

### Working Example 20

### N-(2-Hydroxyethyl)-3-[7-methoxy-2-[3-(trifluoromethyl)benzyl]-1-benzofuran-4-yl]benzamide

3-[7-Methoxy-2-[3-(trifluoromethyl)benzyl]-1-benzofuran-4-yl]benzoic acid obtained in Reference Example 18 and 2-aminoethanol were used in the same manner as in Working Example 3 to obtain the titled compound. Yield: 82%; melting point: 111-112° C. (ethyl acetate-hexane).

¹H-NMR (CDCl₃) δ: 2.50 (1H, br s), 3.65 (2H, q, J=4.5 Hz), 3.85 (2H, t, J=4.5 Hz), 4.03 (3H, s), 4.20 (2H, s), 6.57 (1H, s), 6.64 (1H, br s), 6.84 (1H, d, J=8.4 Hz), 7.22 (1H, d, J=8.4 Hz), 7.38-7.56 (5H, m), 7.65-7.73 (2H, m), 7.96 (1H, d, J=1.5 Hz).

### Working Example 21

### N-(2-Cyanoethyl)-2-[2-[3-(trifluoromethyl)benzyl]-1-benzofuran-4-yl]isonicotinamide

2-[2-[3-(Trifluoromethyl)benzyl]-1-benzofuran-4-yl]isonicotinic acid obtained in Reference Example 21 and 3-aminopropanenitrile were used in the same manner as in Working Example 3 to obtain the titled compound. Yield: 78%; melting point: 114-115° C. (ethyl acetate-hexane).

¹H-NMR (CDCl₃) δ: 2.80 (2H, t, J=6.2 Hz), 3.77 (2H, q, J=6.2 Hz), 4.21 (2H, s), 6.72 (1H, br s), 7.11 (1H, d, J=0.8 Hz), 7.35 (1H, t, J=7.9 Hz), 7.41-7.47 (1H, m), 7.48-7.55 (4H, m), 7.59 (1H, s), 7.68 (1H, dd, J=7.5, 0.8 Hz), 8.10 (1H, d, J=0.8 Hz), 8.88 (1H, dd, J=5.1, 0.8 Hz).

### Working Example 22

### N-(2-Hydroxyethyl)-2-[2-[3-(trifluoromethyl)benzyl]-1-benzofuran-4-yl]isonicotinamide

2-Aminoethanol (10.1 μL, 0.24 mmol) was added to a methanol (2 mL) solution of 2-{2-[3-(trifluoromethyl)benzyl]-1-benzofuran-4-yl}isonicotinic acid (80 mg, 0.20 mmol) obtained in Reference Example 21 and DMTMM (70.7 mg, 0.24 mmol), and the mixture was stirred for 16 hours at room temperature. The reaction solution was concentrated at reduced pressure, and the addition of saturated sodium bicarbonate aqueous solution was followed by extraction with ethyl acetate. The combined organic layers were dried over anhydrous magnesium sulfate, and then concentrated at reduced pressure. The residue was purified by silica gel column chromatography (hexane-ethyl acetate 70:30→0:100) and was recrystallized from ethyl acetate-hexane to give 67 mg of the titled compound (yield 76%) in the form of crystals. Melting point: 79-80° C. (ethyl acetate-hexane).

¹H-NMR (CDCl₃) δ: 2.26 (1H, br s), 3.67 (2H, q, J=5.1 Hz), 3.87 (2H, br s), 4.21 (2H, s), 6.76 (1H, br s), 7.11 (1H, d, J=0.9 Hz), 7.34 (1H, t, J=7.8 Hz), 7.40-7.47 (1H, m), 7.47-7.55 (4H, m), 7.59 (1H, s), 7.66 (1H, dd, J=7.7, 0.8 Hz), 8.10 (1H, d, J=0.8 Hz), 8.84 (1H, dd, J=5.1, 0.8 Hz).

### Working Example 23

### N-(2-Cyanoethyl)-3-[2-[3-(trifluoromethyl)benzyl]furo[3,2-c]pyridin-4-yl]benzamide

3-[2-[3-(Trifluoromethyl)benzyl]furo[3,2-c]pyridin-4-yl]benzoic acid obtained in Reference Example 25 and 3-aminopropanenitrile were used in the same manner as in Working Example 3 to obtain the titled compound. Yield: 91%.

¹H-NMR (CDCl₃) δ: 2.78 (2H, t, J=6.3 Hz), 3.75 (2H, dd, J=12.0, 6.0 Hz), 4.22 (2H, s), 6.78 (1H, s), 6.85 (1H, br s), 7.35 (1H, d, J=5.5 Hz), 7.42-7.64 (5H, m), 7.88 (1H, dd, J=7.7, 1.1 Hz), 8.07 (1H, dd, J=7.7, 1.1 Hz), 8.33 (1H, s), 8.53 (1H, d, J=5.8 Hz).

### Working Example 24

### N-(2-Hydroxyethyl)-3-[2-[3-(trifluoromethyl)benzyl]furo[3,2-c]pyridin-4-yl]benzamide

3-[2-[3-(Trifluoromethyl)benzyl]furo[3,2-c]pyridin-4-yl]benzoic acid obtained in Reference Example 25 and 2-aminoethanol were used in the same manner as in Working Example 22 to obtain the titled compound. Yield: 68%.

¹H-NMR (CDCl₃) δ: 3.61-3.70 (2H, m), 3.82-3.86 (2H, m), 4.22 (2H, s), 6.78 (1H, s), 6.91 (1H, br s), 7.34-7.39 (1H, m), 7.42-7.63 (5H, m), 7.85-7.91 (1H, m), 8.00-8.06 (1H, m), 8.34 (1H, t, J=1.6 Hz), 8.52 (1H, d, J=5.5 Hz), 1H unconfirmed.

### Working Example 25

### N-(2-Cyanoethyl)-3-[2-[3-(trifluoromethyl)benzyl]-1-benzofuran-7-yl]benzamide

3-[2-[3-(Trifluoromethyl)benzyl]-1-benzofuran-7-yl]benzoic acid obtained in Reference Example 28 and 3-aminopropanenitrile were used in the same manner as in Working Example 3 to obtain the titled compound. Yield: 71%; melting point: 154-155° C. (ethyl acetate-hexane).

¹H-NMR (CDCl₃) δ: 2.77 (1H, t, J=6.0 Hz), 3.73 (2H, t, J=6.0 Hz),4.20(2H, s), 6.45 (1H, s), 6.62 (1H, br s), 7.29 (1H, d, J=7.5 Hz), 7.38-7.62 (7H, m), 7.77 (1H, d, J=7.8 Hz), 7.97 (1H, d, J=7.8 Hz), 8.21 (1H, t, J=1.5 Hz).

### Working Example 26

### N-(2-Hydroxyethyl)-3-[2-[3-(trifluoromethyl)benzyl]-1-benzofuran-7-yl]benzamide

3-[2-[3-(Trifluoromethyl)benzyl]-1-benzofuran-7-yl]benzoic acid obtained in Reference Example 28 and 2-aminoethanol were used in the same manner as in Working Example 3 to obtain the titled compound. Yield: 52%; melting point: 140-141° C. (ethyl acetate-hexane).

¹H-NMR (CDCl₃) δ: 2.54 (1H, t, J=5.1 Hz), 3.65 (2H, q, J=5.1 Hz), 3.85 (2H, q, J=5.1 Hz), 4.19 (2H, s), 6.45 (1H, s), 6.63 (1H, br s), 7.29 (1H, d, J=7.8 Hz), 7.38-7.61 (7H, m), 7.77 (1H, dt, J=7.8, 1.5 Hz), 7.95 (1H, dt, J=7.8, 1.5 Hz), 8.20 (1H, t, J=1.5 Hz).

### Working Example 27

### N-[2-(Methylthio)ethyl]-3-[2-[3-(trifluoromethyl)benzyl]-1-benzofuran-7-yl]benzamide

3-[2-[3-(Trifluoromethyl)benzyl]-1-benzofuran-7-yl]benzoic acid obtained in Reference Example 28 and 2-(methylsulfanyl)ethanamine were used in the same manner as in Working Example 3 to obtain the titled compound. Yield: 61%; melting point: 135-138° C. (ethyl acetate-hexane).

¹H-NMR (CDCl₃) δ: 2.15 (3H, s), 2.78 (2H, t, J=6.3 Hz), 3.70 (2H, t, J=6.3 Hz), 4.20 (2H, s), 6.44 (1H, br s), 6.62 (1H, br s), 7.24-7.29 (1H, m), 7.29-7.61 (7H, m), 7.78 (1H, d, J=7.8 Hz), 7.95 (1H, d, J=7.8 Hz), 8.22 (1H, t, J=1.5 Hz).

### Working Example 28

### N-[2-(Methylsulfinyl)ethyl]-3-[2-[3-(trifluoromethyl)benzyl]-1-benzofuran-7-yl]benzamide

While cooled on ice, m-chloroperbenzoic acid (70%) (105 mg, 0.426 mmol) was added to a dichloromethane solution (5.0 mL) of the N-[2-(methylthio)ethyl]-3-[2-[3-(trifluoromethyl)benzyl]-1-benzofuran-7-yl]benzamide

(200 mg, 0.426 mmol) obtained in Working Example 27, and the mixture was stirred for 30 min. The reaction solution was diluted with saturated sodium bicarbonate aqueous solution and extracted with ethyl acetate. The extract was dried over anhydrous sodium sulfate, and then concentrated at reduced pressure, and the residue was purified by silica gel column chromatography (ethyl acetate-methanol 100:0→90:10) to give 160 mg of the titled compound (yield 77%). Melting point: 176-177° C. (ethyl acetate-hexane).

¹H-NMR (CDCl₃) δ: 2.66 (3H, s), 2.82-2.94 (1H, m), 3.13-3.30 (1H, m), 3.97-4.12 (2H, m), 4.21 (2H, s), 6.42 (1H, s), 7.24-7.30 (1H, m), 7.35-7.57 (7H, m), 7.62 (1H, s), 7.78 (1H, d, J=7.5 Hz), 7.96 (1H, d, J=7.5 Hz), 8.24 (1H, s).

### Working Example 29

### N-[2-Hydroxy-1-(hydroxymethyl)ethyl]-3-[2-[3-(trifluoromethyl)benzyl]-1-benzofuran-7-yl]benzamide

2-Aminopropane-1,3-diol (109 mg, 1.2 mmol) was added to a methanol (5 mL) solution of 3-[2-[3-(trifluoromethyl)benzyl]-1-benzofuran-7-yl]benzoic acid (400 mg, 1.0 mmol) obtained in Reference Example 28 and DMTMM (324 mg, 1.1 mmol), and the mixture was stirred for 5 hours at room temperature. The reaction solution was concentrated at reduced pressure, and the addition of saturated sodium bicarbonate aqueous solution was followed by extraction with ethyl acetate. The organic layer was dried over anhydrous magnesium sulfate and then concentrated at reduced pressure. The residue was purified by silica gel column chromatography (ethyl acetate-hexane 5:95→60:40) and recrystallized from ethyl acetate-hexane to give 300 mg of the titled compound (yield 64%). Melting point: 105-107° C. (ethyl acetate-hexane).

¹H-NMR (CDCl₃) δ: 2.78 (2H, br s), 3.82-4.02 (4H, m), 4.14-4.24 (3H, m), 6.43 (1H, s), 7.02 (1H, d, J=6.6 Hz), 7.24-7.30 (1H, m), 7.28-7.55 (6H, m), 7.60 (1H, s), 7.78 (1H, d, J=7.8 Hz), 7.95 (1H, d, J=7.8 Hz), 8.24 (1H, s).

### Working Example 30

### N-(2-Cyanoethyl)-3-[3-methyl-2-[3-(trifluoromethyl)benzyl]-1-benzofuran-7-yl]benzamide

3-[3-Methyl-2-[3-(trifluoromethyl)benzyl]-1-benzofuran-7-yl]benzoic acid obtained in Reference Example 32 and 3-aminopropanenitrile were used in the same manner as in Working Example 3 to obtain the titled compound. Yield: 65%; melting point: 158-159° C. (ethyl acetate-hexane).

¹H-NMR (CDCl₃) δ: 2.27 (3H, s), 2.75 (2H, t, J=6.0 Hz), 3.71 (2H, q, J=6.0 Hz), 4.18 (2H, s), 6.57 (1H, br s), 7.31 (1H, t, J=7.5 Hz), 7.39-7.57 (7H, m), 7.76 (1H, d, J=7.5 Hz), 7.96 (1H, d, J=7.5 Hz), 8.18 (1H, t, J=1.5 Hz).

### Working Example 31

### N-(2-Hydroxyethyl)-3-[3-methyl-2-[3-(trifluoromethyl)benzyl]-1-benzofuran-7-yl]benzamide

3-[3-Methyl-2-[3-(trifluoromethyl)benzyl]-1-benzofuran-7-yl]benzoic acid obtained in Reference Example 32 and 2-aminoethanol were used in the same manner as in Working Example 3 to obtain the titled compound. Yield: 62%; melting point: 158-159° C. (ethyl acetate-hexane).

¹H-NMR (CDCl₃) δ: 2.26 (3H, s), 2.57 (1H, br s), 3.63 (2H, q, J=5.7 Hz), 3.83 (2H, q, J=5.7 Hz), 4.17 (2H, s), 6.61 (1H, br s), 7.30 (1H, t, J=7.8 Hz), 7.40-7.56 (7H, m), 7.75 (1H, d, J=7.8 Hz), 7.93 (1H, d, J=7.8 Hz), 8.18 (1H, t, J=1.5 Hz).

### Working Example 32

### N-[2-(Methylthio)ethyl]-3-[3-methyl-2-[3-(trifluoromethyl)benzyl]-1-benzofuran-7-yl]benzamide

3-[3-Methyl-2-[3-(trifluoromethyl)benzyl]-1-benzofuran-7-yl]benzoic acid obtained in Reference Example 32 and 2-(methylsulfanyl)ethanamine were used in the same manner as in Working Example 3 to obtain the titled compound. Yield: 78%; melting point: 138-139° C. (ethyl acetate-hexane).

¹H-NMR (CDCl₃) δ: 2.14 (3H, s), 2.26 (3H, s), 2.76 (2H, t, J=6.3 Hz), 3.69 (2H, q, J=5.9 Hz), 4.18 (2H, s), 6.59 (1H, br s), 7.32 (1H, t, J=7.6 Hz), 7.41-7.51 (5H, m), 7.54 (1H, d, J=8.1 Hz), 7.57 (1H, s), 7.74-7.81 (1H, m), 7.92-7.98 (1H, m), 8.20 (1H, t, J=1.6 Hz).

### Working Example 33

### N-[2-(Methylsulfinyl)ethyl]-3-[3-methyl-2-[3-(trifluoromethyl)benzyl]-1-benzofuran-7-yl]benzamide

While cooled on ice, m-chloroperbenzoic acid (224 mg, 0.91 mmol) was added to a dichloromethane (4 mL) solution of the N-[2-(methylthio)ethyl]-3-[3-methyl-2-[3-(trifluoromethyl)benzyl]-1-benzofuran-7-yl]benzamide (400 mg, 0.83 mmol) obtained in Working Example 32, and the mixture was stirred for 1 hour. The addition of saturated sodium bicarbonate aqueous solution to the reaction solution was followed by extraction with ethyl acetate. The organic layer was dried over anhydrous sodium sulfate and then concentrated at reduced pressure. The residue was purified by basic silica gel column chromatography (hexane-ethyl acetate 50:50→0:100), and the fractions containing components of higher polarity were concentrated and recrystallized from ethyl acetate-hexane to give 200 mg of the titled compound (yield 48%) in the form of crystals. Melting point: 146-147° C.

¹H-NMR (CDCl₃) δ: 2.26 (3H, s), 2.66 (3H, s), 2.84-2.94 (1H, m), 3.12-3.24 (1H, m), 3.97-4.11 (2H, m), 4.19 (2H, s), 7.28-7.37 (2H, m), 7.38-7.55 (6H, m), 7.58 (1H, s), 7.77 (1H, d, J=8.3 Hz), 7.96 (1H, d, J=7.6 Hz), 8.23 (1H, s).

### Working Example 34

### N-[2-(Methylsulfonyl)ethyl]-3-[3-methyl-2-[3-(trifluoromethyl)benzyl]-1-benzofuran-7-yl]benzamide

While cooled on ice, m-chloroperbenzoic acid (224 mg, 0.91 mmol) was added to a dichloromethane (4 mL) solution of the N-[2-(methylthio)ethyl]-3-[3-methyl-2-[3-(trifluoromethyl)benzyl]-1-benzofuran-7-yl]benzamide (400 mg, 0.83 mmol) obtained in Working Example 32, and the mixture was stirred for 1 hour. The addition of saturated sodium bicarbonate aqueous solution to the reaction solution was followed by extraction with ethyl acetate. The organic layer was dried over anhydrous sodium sulfate and then concentrated at reduced pressure. The residue was purified by basic silica gel column chromatography (hexane-ethyl acetate 50:50→0:100), and the fractions containing components of lower polarity were concentrated and recrystallized from ethyl acetate-hexane to give 85 mg of the titled compound (yield 16%) in the form of crystals. Melting point: 162-163° C.

¹H-NMR (CDCl₃) δ: 2.26 (3H, s), 2.98 (3H, s), 3.32-3.42 (2H, m), 3.97-4.08 (2H, m), 4.19 (2H, s), 7.01 (1H, br s), 7.28-7.36 (1H, m), 7.40-7.56 (6H, m), 7.58 (1H, s), 7.77 (1H, d, J=8.3 Hz), 7.96 (1H, d, J=7.6 Hz), 8.23 (1H, s).

### Working Example 35

### N-(2-Cyanoethyl)-2-[3-methyl-2-[3-(trifluoromethyl)benzyl]-1-benzofuran-7-yl]pyridine-4-carboxamide

2-[3-Methyl-2-[3-(trifluoromethyl)benzyl]-1-benzofuran-7-yl]pyridine-4-carboxylic acid obtained in Reference Example 34 and 3-aminopropanenitrile were used in the same manner as in Working Example 3 to obtain the titled compound. Yield: 75%; melting point: 200-201° C. (THF-hexane).

¹H-NMR (DMSO-d₆) δ: 2.31 (3H, s), 2.84 (2H, t, J=6.2 Hz), 3.58 (2H, q, J=6.2 Hz), 4.35 (2H, s), 7.35-7.44 (1H, m), 7.54-7.62 (2H, m), 7.63-7.68 (1H, m), 7.71-7.78 (3H, m), 8.08 (1H, dd, J=7.6, 1.1 Hz), 8.68 (1H, s), 8.88 (1H, dd, J=4.9, 1.1 Hz), 9.21 (1H, t, J=6.2 Hz).

### Working Example 36

### N-(2-Hydroxyethyl)-2-[3-methyl-2-[3-(trifluoromethyl)benzyl]-1-benzofuran-7-yl]pyridine-4-carboxamide

2-[3-Methyl-2-[3-(trifluoromethyl)benzyl]-1-benzofuran-7-yl]pyridine-4-carboxylic acid obtained in Reference Example 34 and 2-aminoethanol were used in the same manner as in Working Example 3 to obtain the titled compound. Yield: 66%; melting point: 205-206° C. (THF-hexane).

¹H-NMR (DMSO-d₆) δ: 2.31 (3H, s), 3.41 (2H, q, J=5.9 Hz), 3.57 (2H, q, J=5.9 Hz), 4.35 (2H, s), 4.78 (1H, t, J=5.9 Hz), 7.38 (1H, t, J=7.8 Hz), 7.53-7.68 (3H, m), 7.71-7.81 (3H, m), 8.07 (1H, dd, J=7.8, 1.1 Hz), 8.66 (1H, s), 8.79 (1H, t, J=5.9 Hz), 8.84 (1H, d, J=4.9 Hz).

### Working Example 37

### N-(2-Cyanoethyl)-3-[4-fluoro-2-[3-(trifluoromethyl)benzyl]-1-benzofuran-7-yl]benzamide

3-[4-Fluoro-2-[3-(trifluoromethyl)benzyl]-1-benzofuran-7-yl]benzoic acid obtained in Reference Example 37 and 3-aminopropanenitrile were used in the same manner as in Working Example 3 to obtain the titled compound. Yield: 66%; melting point: 158-159° C. (ethyl acetate-hexane).

¹H-NMR (CDCl₃) δ: 2.76 (2H, t, J=6.0 Hz), 3.72 (2H, q, J=6.0 Hz), 4.19 (2H, s), 6.53 (1H, s), 6.68 (1H, br s), 6.97 (1H, t, J=8.7 Hz), 7.34 (1H, dd, J=8.7, 4.8 Hz), 7.40-7.60 (5H, m), 7.75 (1H, d, J=7.8 Hz), 7.90 (1H, d, J=8.1 Hz), 8.16 (1H, d, J=1.5 Hz).

### Working Example 38

### 3-[4-Fluoro-2-[3-(trifluoromethyl)benzyl]-1-benzofuran-7-yl]-N-(2-hydroxyethyl)benzamide

3-[4-Fluoro-2-[3-(trifluoromethyl)benzyl]-1-benzofuran-7-yl]benzoic acid obtained in Reference Example 37 and 2-aminoethanol were used in the same manner as in Working Example 3 to obtain the titled compound. Yield: 60%; melting point: 159-160° C. (ethyl acetate-hexane).

¹H-NMR (CDCl₃) δ: 2.49 (1H, br s), 3.66 (2H, q, J=5.4 Hz), 3.86 (2H, q, J=5.4 Hz), 4.19 (2H, s), 6.53 (1H, s), 6.62 (1H, br s), 6.98 (1H, t, J=8.7 Hz), 7.34 (1H, dd, J=8.7, 5.4 Hz), 7.43-7.60 (5H, m), 7.76 (1H, d, J=7.5 Hz), 7.88 (1H, d, J=7.8 Hz), 8.15 (1H, s).

### Working Example 39

### 4-Hydroxy-N-(3-[2-[3-(trifluoromethyl)benzyl]-1-benzofuran-7-yl]phenyl)butanamide

A toluene solution (5.0 mL) of 3-[2-[3-(trifluoromethyl)benzyl]-1-benzofuran-7-yl]aniline (300 mg, 0.82 mmol) obtained in Reference Example 38 and succinic anhydride (100 mg, 1.0 mmol) was heated to reflux for 2 hours. The reaction solution was concentrated at reduced pressure to give a 4-oxo-4-[(3-[2-[3-(trifluoromethyl)benzyl]-1-benzofuran-7-yl]phenyl)amino]butanoic acid crude product. A mixture of this compound, sodium acetate (202 mg, 2.46 mmol), and acetic anhydride (5.0 mL) was heated to 70° C. for 5 hours. The addition of water to the reaction solution was followed by extraction with ethyl acetate. The organic layer was washed with water, dried over anhydrous sodium sulfate, and then concentrated at reduced pressure to give a 1-(3-[2-[3-(trifluotomethyl)benzyl]-1-benzofuran-7-yl]phenyl)pyrrolidine-2,5-dione crude product. Sodium borohydride (310 mg, 8.20 mmol) was added at room temperature to an ethanol (7 mL) solution of this compound, and the mixture was heated to 60° C. for 1 hour. The reaction solution was concentrated at reduced pressure, and the addition of water was followed by extraction with ethyl acetate. The organic layer was washed with water and saturated brine, then dried over anhydrous sodium sulfate, and concentrated at reduced pressure. The residue was purified by silica gel column chromatography (hexane-ethyl acetate 30:70→0:100) to give 160 mg of the titled compound (yield 43%). Melting point: 134-135° C. (ethyl acetate-hexane).

¹H-NMR (CDCl₃) δ: 1.90-2.11 (2H, m), 2.34 (1H, br s), 2.56 (2H, t, J=6.9 Hz), 3.70-3.90 (2H, m), 4.19 (2H, s), 6.43 (1H, s), 7.20-7.72 (11H, m), 7.87 (1H, s).

### Working Example 40

### N-(2-Cyanoethyl)-3-[2-[3-(trifluoromethyl)benzyl]-2,3-dihydro-1-benzofuran-7-yl]benzamide

3-[2-[3-(Trifluoromethyl)benzyl]-2,3-dihydro-1-benzofuran-7-yl]benzoic acid obtained in Reference Example 39 and 3-aminopropanenitrile were used in the same manner as in Working Example 3 to obtain the titled compound. Yield: 68%; melting point: 145-146° C. (ethyl acetate-hexane).

¹H-NMR (CDCl₃) δ: 2.74 (2H, t, J=6.0 Hz), 2.97-3.06 (2H, m), 3.18-3.39 (2H, m), 3.58-3.82 (2H, m), 5.00-5.18 (1H, m), 6.61 (1H, br s), 6.94 (1H, t, J=7.5 Hz), 7.15 (1H, d, J=8.7 Hz), 7.29-7.60 (6H, m), 7.69 (1H, d, J=6.6 Hz), 7.86 (1H, t, J=6.9 Hz), 8.08 (1H, s).

### Working Example 41

### N-(2-Hydroxyethyl)-3-[2-[3-(trifluoromethyl)benzyl]-2,3-dihydro-1-benzofuran-7-yl]benzamide

3-[2-[3-(Trifluoromethyl)benzyl]-2,3-dihydro-1-benzofuran-7-yl]benzoic acid obtained in Reference Example 39 and 2-aminoethanol were used in the same manner as in Working Example 3 to obtain the titled compound. Yield: 55%; melting point: 161-162° C. (ethyl acetate-hexane).

¹H-NMR (CDCl₃) δ: 2.60 (1H, br s), 2.97-3.08 (2H, m), 3.18-3.38 (2H, m), 3.63 (2H, q, J=5.1 Hz), 3.83 (2H, q, J=5.1 Hz), 5.00-5.13 (1H, m), 6.60 (1H, br s), 6.93 (1H, t, J=7.5 Hz), 7.14 (1H, d, J=7.5 Hz), 7.32 (1H, d, J=7.8 Hz), 7.28-7.52 (5H, m), 7.70 (1H, d, J=7.5 Hz), 7.85 (1H, t, J=6.9 Hz), 8.08 (1H, s).

### Working Example 42

### 3-[2-(3-Fluorobenzyl)-1-benzofuran-7-yl]-N-(2-hydroxyethyl)benzamide

3-[2-(3-Fluorobenzyl)-1-benzofuran-7-yl]benzoic acid obtained in Reference Example 42 and 2-aminoethanol were used in the same manner as in Working Example 3 to obtain the titled compound. Yield: 13%; melting point: 159-160° C. (ethyl acetate-hexane).

¹H-NMR (CDCl₃) δ: 2.64 (1H, br s), 3.64 (2H, q, J=5.4 Hz), 3.78-3.86 (2H, m), 4.12 (2H, s), 6.45 (1H, s), 6.65 (1H, br s), 6.91-6.99 (1H, m), 7.02 (1H, d, J=9.9 Hz), 7.09 (1H, d, J=7.2 Hz), 7.23-7.36 (2H, m), 7.40 (1H, d, J=6.6 Hz), 7.47 (1H, d, J=7.5 Hz), 7.53 (1H, t, J=7.8 Hz), 7.78 (1H, d, J=8.1 Hz), 7.95 (1H, d, J=7.8 Hz), 8.19 (1H, s).

### Working Example 43

### 3-[2-(3-Methoxybenzyl)-1-benzofuran-7-yl]-N-(2-pyrrolidin-1-ylethyl)benzamide

3-[2-(3-Methoxybenzyl)-1-benzofuran-7-yl]benzoic acid obtained in Reference Example 45 and 2-pyrrolidin-1-yl ethanolamine were used in the same manner as in Working Example 3 to obtain the titled compound. Yield: 31%; melting point: 101-102° C. (ethyl acetate-hexane).

¹H-NMR (CDCl₃) δ: 1.70-1.90 (4H, m), 2.42-2.63 (4H, m), 2.71 (2H, t, J=6.0 Hz), 3.58 (2H, q, J=6.0 Hz), 3.78 (3H, s), 4.10 (2H, s), 6.40 (1H, s), 6.76-6.92 (4H, m), 7.23-7.29 (2H, m), 7.40 (1H, dd, J=7.8, 1.5 Hz), 7.45 (1H, dd, J=7.8, 1.5 Hz), 7.53 (1H, t, J=7.8 Hz), 7.77 (1H, d, J=7.5 Hz), 7.97 (1H, dd, J=6.0, 0.9 Hz), 8.21 (1H, s).

### Working Example 44

### N-(6-Fluoropyridin-3-yl)-3-[2-(3-methoxybenzyl)-1-benzofuran-7-yl]benzamide

3-[2-(3-Methoxybenzyl)-1-benzofuran-7-yl]benzoic acid obtained in Reference Example 45 and 6-fluoropyridin-3-amine were used in the same manner as in Working Example 8 to obtain the titled compound. Yield: 55%; melting point: 196-199° C. (ethyl acetate-hexane).

¹H-NMR (CDCl₃) δ: 3.75 (3H, s), 4.11 (2H, s), 6.48 (1H, s), 6.74 (1H, d, J=7.8 Hz), 6.89 (1H, d, J=6.3 Hz), 6.98 (1H, dd, J=9.0, 3.0 Hz), 7.20 (1H, t, J=7.8 Hz), 7.30 (1H, d, J=7.5 Hz), 7.41 (1H, dd, J=7.5, 1.2 Hz), 7.50 (1H, d, J=7.8 Hz), 7.61 (1H, t, J=7.5 Hz), 7.82-8.04 (2H, m), 8.03 (1H, d, J=7.2 Hz), 8.23-8.40 (3H, m).

### Working Example 45

### 3-[2-(3-Methoxybenzyl)-1-benzofuran-7-yl]-N-(tetrahydro-2H-pyran-4-yl)benzamide

3-[2-(3-Methoxybenzyl)-1-benzofuran-7-yl]benzoic acid obtained in Reference Example 45 and tetrahydro-2H-pyran-4-amine were used in the same manner as in Working Example 3 to obtain the titled compound. Yield: 82%; melting point: 146-147° C. (ethyl acetate-hexane).

¹H-NMR (CDCl₃) δ: 1.43-1.68 (2H, m), 1.95-2.10 (2H, m), 3.55 (2H, t, J=11.7 Hz), 3.70 (3H, s), 3.92-4.30 (5H, m), 5.92-6.07 (1H, m), 6.44 (1H, s), 6.78-6.92 (3H, m), 7.20-7.30 (2H, m), 7.40 (1H, d, J=6.3 Hz), 7.47 (1H, d, J=7.5 Hz), 7.54 (1H, t, J=7.8 Hz), 7.77 (1H, d, J=7.8 Hz), 7.97 (1H, d, J=7.5 Hz), 8.17 (1H, s).

### Working Example 46

### 3-[2-(3,4-dimethoxybenzyl)-1-benzofuran-7-yl]-N-(2-pyrrolidin-1-ylethyl)benzamide

3-[2-(3,4-Dimethoxybenzyl)-1-benzofuran-7-yl]benzoic acid obtained in Reference Example 48 and 2-pyrrolidin-1-yl ethanamine were used in the same manner as in Working Example 3 to obtain the titled compound. Yield: 62%; melting point: 119-120° C. (ethyl acetate-hexane).

¹H-NMR (CDCl₃) δ: 1.75-1.80 (4H, m), 2.51-2.61 (4H, m), 2.71 (2H, t, J=6.0 Hz), 3.58 (2H, q, J=6.0 Hz), 3.84 (3H, s), 3.87 (3H, s), 4.07 (2H, s), 6.38 (1H, s), 6.77-6.90 (4H, m), 7.21-7.28 (1H, m), 7.40 (1H, dd, J=7.5, 1.5 Hz), 7.46 (1H, dd, J=7.8, 1.5 Hz), 7.53 (1H, t, J=7.8 Hz), 7.77 (1H, d, J=7.8 Hz), 7.97 (1H, dd, J=7.8, 1.5 Hz), 8.23 (1H, d, J=1.5 Hz).

### Working Example 47

### 3-[2-(3,4-dimethoxybenzyl)-2,3-dihydro-1-benzofuran-7-yl]-N-(2-pyrrolidin-1-ylethyl)benzamide

Triethylsilane (0.21 mL, 1.32 mmol) was added at room temperature to a trifluoroacetic acid (5 mL) mixture of 3-[2-(3,4-dimethoxybenzyl)-1-benzofuran-7-yl]-N-(2-pyrrolidin-1-ylethyl)benzamide (160 mg, 0.33 mmol) obtained in Working Example 46, and the mixture was heated to reflux for 3 hours. The reaction solution was concentrated at reduced pressure, and the subsequent addition of saturated sodium bicarbonate aqueous solution to the residue was followed by extraction with ethyl acetate. The organic layer was washed with water and saturated brine, then dried over anhydrous sodium sulfate, and concentrated at reduced pressure. The resulting residue was purified by silica gel column chromatography (methanol-ethyl acetate 0:100→5:95) to give 65 mg of the titled compound (yield 40%). Melting point: 134-135° C. (ethyl acetate-hexane).

¹H-NMR (CDCl₃) δ: 1.72-1.82 (4H, m), 2.41-2.61 (4H, m), 2.69 (2H, t, J=6.0 Hz), 2.90 (1H, dd, J=14.4, 6.0 Hz), 3.01 (1H, dd, J=15.6, 7.2 Hz), 3.15 (1H, dd, J=14.4, 6.3 Hz), 3.28 (1H, dd, J=15.6, 8.4 Hz), 3.56 (2H, q, J=6.0 Hz), 3.74 (3H, s), 3.85 (3H, s), 5.00-5.10 (1H, m), 6.74-6.82 (4H, m), 6.92 (1H, d, J=7.5 Hz), 7.13 (1H, t, J=7.2 Hz), 7.31 (1H, d, J=7.2 Hz), 7.45 (1H, t, J=7.8 Hz), 7.80 (1H, d, J=7.8 Hz), 7.86 (1H, d, J=7.5 Hz), 8.12 (1H, s).

### Working Example 48

### N-(2-Cyanoethyl)-3-[2-[3-(trifluoromethyl)benzyl]-1H-indol-7-yl]benzamide

1 N sodium hydroxide aqueous solution (2.83 mL, 2.83 mmol) was added to an ethanol (7 mL) and THF (3 mL) mixture of ethyl 3-[2-[3-(trifluoromethyl)benzyl]-1H-indol-7-yl]benzoate (0.40 g, 0.94 mmol) obtained in Reference Example 51, and the mixture was stirred for 1 hour. The reaction solution was neutralized with the addition of 1 N hydrochloric acid (2.83 mL, 2.83 mmol), and the solvent was distilled off at reduced pressure. WSC (0.21 g, 1.23 mmol) and HOBt (0.17 g, 1.23 mmol) were added to a mixture of the residue, 3-aminopropanenitrile (0.081 mL, 1.13 mmol), and DMF (5 mL), and the mixture was stirred for 15 hours. The reaction solution was diluted with water and extracted with ethyl acetate. The extract was washed with water and dried over anhydrous magnesium sulfate, and the solvent was then distilled off at reduced pressure. The residue was purified by HPLC (Waters Preparative HPLC System; column: Waters SunFire Column C18 (30×50 mm S−5 μm); eluate A: distilled water (0.1% TFA), eluate B: MeCN (0.1% TFA); 40-100% Gradient) to give 80.8 mg of the titled compound (yield 19%) in the form of crystals.

Melting point: 174-178° C.

¹H-NMR (CDCl₃) δ: 2.71 (2H, t, J=6.3 Hz), 3.67 (2H, t, J=6.0 Hz), 4.18 (2H, s), 6.33 (1H, s), 6.65 (1H, m), 7.10-7.25 (2H, m), 7.35-7.65 (6H, m), 7.54 (2H, d, J=7.8 Hz), 8.01 (1H, s), 8.33 (1H, s).

### Working Example 49

### N-(2-Hydroxyethyl)-3-[2-[3-(trifluoromethyl)benzyl]-1H-indol-7-yl]benzamide

Ethyl 3-[2-[3-(trifluoromethyl)benzyl]-1H-indol-7-yl]benzoate obtained in Reference Example 51 and 2-aminoethanol were used in the same manner as in Working Example 48 to obtain the titled compound. 20% yield, oily substance.

¹H-NMR (CDCl₃) δ: 2.90 (1H, br s), 3.60 (2H, q, J=5.0 Hz), 3.78 (2H, t, J=4.7 Hz), 5.40 (2H, s), 6.60 (1H, d, J=3.0 Hz), 6.77 (1H, br s), 7.14 (1H, d, J=3.0 Hz), 7.18 (1H, d, J=7.2 Hz), 7.30-7.50 (5H, m), 7.50 (1H, d, J=7.5 Hz), 7.60-7.75 (3H, m).

### Working Example 50

### N-(2-Cyanoethyl)-3-[2-[3-(trifluoromethyl)benzyl]-2H-indazol-7-yl]benzamide

3-[2-[3-(Trifluoromethyl)benzyl]-2H-indazol-7-yl]benzoic acid obtained in Reference Example 54 and 3-aminopropanenitrile were used in the same manner as in Working Example 3 to obtain the titled compound. Yield: 84%, melting point: 174-175° C.

¹H-NMR (DMSO-d₆) δ: 2.81 (2H, t, J=6.4 Hz), 3.53 (2H, q, J=6.4 Hz), 5.83 (2H, s), 7.20 (1H, dd, J=8.3, 7.0 Hz), 7.52-7.62 (4H, m), 7.66-7.72 (1H, m), 7.75-7.87 (3H, m), 8.26-8.33 (1H, m), 8.44 (1H, t, J=1.6 Hz), 8.65 (1H, s), 8.93 (1H, t, J=6.4 Hz).

### Working Example 51

### N-(2-Hydroxyethyl)-3-[2-[3-(trifluoromethyl)benzyl]-2H-indazol-7-yl]benzamide

3-[2-[3-(Trifluoromethyl)benzyl]-2H-indazol-7-yl]benzoic acid obtained in Reference Example 54 and 2-aminoethanol were used in the same manner as in Working Example 3 to obtain the titled compound. Yield: 78%; melting point: 136-137° C. (ethyl acetate-hexane).

¹H-NMR (DMSO-d₆) δ: 3.33-3.41 (2H, m), 3.54 (2H, q, J=6.0 Hz), 4.75 (1H, t, J=6.0 Hz), 5.83 (2H, s), 7.19 (1H, dd, J=8.3, 7.2 Hz), 7.49-7.65 (4H, m), 7.64-7.73 (1H, m), 7.74-7.87 (3H, m), 8.27 (1H, d, J=8.3 Hz), 8.40 (1H, s), 8.52 (1H, t, J=6.0 Hz), 8.65 (1H, s).

### Working Example 52

### N-(2-Cyanoethyl)-3-[2-[3-(trifluoromethyl)benzyl]-2H-indazol-4-yl]benzamide

3-[2-[3-(Trifluoromethyl)benzyl]-2H-indazol-4-yl]benzoic acid obtained in Reference Example 57 and 3-aminopropanenitrile were used in the same manner as in Working Example 3 to obtain the titled compound. Yield: 74%; melting point: 187-188° C. (ethyl acetate-hexane).

¹H-NMR (DMSO-d₆) δ: 2.81 (2H, t, J=6.4 Hz), 3.54 (2H, q, J=6.4 Hz), 5.79 (2H, s), 7.26 (1H, d, J=6.2 Hz), 7.38 (1H, dd, J=8.6, 6.2 Hz), 7.54-7.73 (5H, m), 7.79 (1H, s), 7.88-7.96 (2H, m), 8.19 (1H, s), 8.80 (1H, d, J=0.8 Hz), 8.98 (1H, t, J=6.4 Hz).

### Working Example 53

### N-(2-Hydroxyethyl)-3-[2-[3-(trifluoromethyl)benzyl]-2H-indazol-4-yl]benzamide

3-[2-[3-(Trifluoromethyl)benzyl]-2H-indazol-4-yl]benzoic acid obtained in Reference Example 57 and 2-aminoethanol were used in the same manner as in Working Example 3 to obtain the titled compound. Yield: 74%; melting point: 136-137° C. (ethyl acetate-hexane).

¹H-NMR (DMSO-d₆) δ: 3.31-3.41 (2H, m), 3.50-3.58 (2H, m), 4.74 (1H, t, J=5.7 Hz), 5.79 (2H, s), 7.26 (1H, d, J=6.2 Hz), 7.33-7.42 (1H, m), 7.53-7.72 (5H, m), 7.79 (1H, s), 7.85-7.94 (2H, m), 8.18 (1H, t, J=1.6 Hz), 8.57 (1H, t, J=5.7 Hz), 8.80 (1H, d, J=0.8 Hz).

### Working Example 54

### N-(2-Cyanoethyl)-3-[3-methyl-2-[3-(trifluoromethyl)benzyl]-2H-indazol-7-yl]benzamide

3-[3-Methyl-2-[3-(trifluoromethyl)benzyl]-2H-indazol-7-yl]benzoic acid obtained in Reference Example 61 and 3-aminopropanenitrile were used in the same manner as in Working Example 3 to obtain the titled compound in solid form. Yield: 84%; melting point: 196-197° C. (THF-hexane).

¹H-NMR (DMSO-d₆) δ: 2.64 (3H, s), 2.80 (2H, t, J=6.4 Hz), 3.53 (2H, q, J=6.4 Hz), 5.82 (2H, s), 7.15 (1H, dd, J=8.3, 7.0 Hz), 7.36-7.43 (1H, m), 7.53-7.62 (3H, m), 7.63-7.70 (2H, m), 7.74 (1H, d, J=7.0 Hz), 7.79-7.85 (1H, m), 8.31-8.38 (1H, m), 8.41-8.48 (1H, m), 8.94 (1H, t, J=6.4 Hz).

### Working Example 55

### N-(2-Hydroxyethyl)-3-[3-methyl-2-[3-(trifluoromethyl)benzyl]-2H-indazol-7-yl]benzamide

3-[3-Methyl-2-[3-(trifluoromethyl)benzyl]-2H-indazol-7-yl]benzoic acid obtained in Reference Example 61 and 2-aminoethanol were used in the same manner as in Working Example 3 to obtain the titled compound. Yield: 70%; melting point: 142-143° C. (ethyl acetate-hexane).

¹H-NMR (DMSO-d₆) δ: 2.64 (3H, s), 3.29-3.40 (2H, m), 3.53 (2H, q, J=6.0 Hz), 4.75 (1H, t, J=6.0 Hz), 5.82 (2H, s), 7.15 (1H, dd, J=8.4, 7.1 Hz ), 7.40 (1H, d, J=7.1 Hz), 7.49-7.61 (3H, m), 7.64-7.76 (3H, m), 7.78-7.85 (1H, m), 8.30-8.36 (1H, m), 8.38-8.45 (1H, m), 8.53 (1H, t, J=6.0 Hz).

### Working Example 56

### N-[2-(Dimethylamino)ethyl]-3-[2-[3-(trifluoromethyl)benzyl]-2H-indazol-7-yl]benzamide

3-[2-[3-(Trifluoromethyl)benzyl]-2H-indazol-7-yl]benzoic acid obtained in Reference Example 54 and N,N-dimethylethane-1,2-diamine were used in the same manner as in Working Example 3 to obtain the titled compound. Yield: 72%; melting point: 102-103° C. (ethyl acetate-hexane).

¹H-NMR (DMSO-d₆) δ: 2.18 (6H, s), 2.42 (2H, t, J=6.9 Hz ), 3.34-3.43 (2H, m), 5.84 (2H, s), 7.19 (1H, dd, J=8.2, 7.1 Hz), 7.50-7.63 (4H, m), 7.65-7.72 (1H, m), 7.73-7.85 (3H, m), 8.21-8.30 (1H, m), 8.40 (1H, s), 8.47 (1H, t, J=6.9 Hz), 8.65 (1H, s).

### Working Example 57

### N-(2-Cyanoethyl)-3-[2-(3-methoxybenzyl)-2H-indazol-4-yl]benzamide

3-[2-(3-Methoxybenzyl)-2H-indazol-4-yl]benzoic acid obtained in Reference Example 64 and 3-aminopropanenitrile were used in the same manner as in Working Example 3 to obtain the titled compound. Yield: 69%; melting point: 176-177° C. (THF-hexane).

¹H-NMR (DMSO-d₆) δ: 2.81 (2H, t, J=6.4 Hz), 3.54 (2H, q, J=6.4 Hz), 3.72 (3H, s), 5.63 (2H, s), 6.83-6.98 (3H, m), 7.21-7.30 (2H, m), 7.37 (1H, dd, J=8.6, 6.9 Hz), 7.60-7.69 (2H, m), 7.84-7.95 (2H, m), 8.16-8.21 (1H, m), 8.71 (1H, d, J=0.8 Hz), 8.98 (1H, t, J=6.4 Hz).

### Working Example 58

### N-(2-Hydroxyethyl)-3-[2-(3-methoxybenzyl)-2H-indazol-4-yl]benzamide

3-[2-(3-Methoxybenzyl)-2H-indazol-4-yl]benzoic acid obtained in Reference Example 64 and 2-aminoethanol were used in the same manner as in Working Example 3 to obtain the titled compound. Yield: 52%; melting point: 176-177° C. (ethyl acetate-hexane).

¹H-NMR (DMSO-d₆) δ: 3.30-3.41 (2H, m), 3.54 (2H, q, J=6.0 Hz), 3.72 (3H, s), 4.75 (1H, t, J=6.0 Hz ), 5.63 (2H, s), 6.82-6.97 (3H, m), 7.19-7.29 (2H, m), 7.36 (1H, dd J=8.6, 6.9 Hz), 7.57-7.67 (2H, m), 7.82-7.93 (2H, m), 8.14-8.19 (1H, m), 8.57 (1H, t, J=6.0 Hz), 8.72 (1H, d, J=0.8 Hz).

### Working Example 59

### N-(2-Cyanoethyl)-3-[2-(2-fluorobenzyl)-2H-indazol-4-yl]benzamide

3-[2-(2-Fluorobenzyl)-2H-indazol-4-yl]benzoic acid obtained in Reference Example 67 and 3-aminopropanenitrile were used in the same manner as in Working Example 3 to obtain the titled compound. Yield: 81%; melting point: 165-166° C. (THF-hexane).

¹H-NMR (DMSO-d₆) δ: 2.81 (2H, t, J=6.5 Hz), 3.54 (2H, q, J=6.5 Hz), 5.75 (2H, s), 7.13-7.45 (6H, m), 7.60-7.70 (2H, m), 7.86-7.95 (2H, m), 8.18 (1H, t, J=1.6 Hz), 8.70 (1H, s), 8.96 (1H, t, J=6.5 Hz).

### Working Example 60

### 3-[2-(2-Fluorobenzyl)-2H-indazol-4-yl]-N-(2-hydroxyethyl)benzamide

3-[2-(2-Fluorobenzyl)-2H-indazol-4-yl]benzoic acid obtained in Reference Example 67 and 2-aminoethanol were used in the same manner as in Working Example 3 to obtain the titled compound. Yield: 58%; melting point: 159-160° C. (THF-hexane).

¹H-NMR (DMSO-d₆) δ: 3.37 (2H, q, J=6.0 Hz), 3.54 (2H, q, J=6.0 Hz), 4.73 (1H, t, J=6.0 Hz), 5.75 (2H, s), 7.14-7.43 (6H, m), 7.56-7.66 (2H, m), 7.83-7.93 (2H, m), 8.17 (1H, s), 8.55 (1H, t, J=6.0 Hz), 8.70 (1H, s).

### Working Example 61

### N-(2-Cyanoethyl)-3-[2-(3-fluorobenzyl)-2H-indazol-4-yl]benzamide

3-[2-(3-Fluorobenzyl)-2H-indazol-4-yl]benzoic acid obtained in Reference Example 70 and 3-aminopropanenitrile were used in the same manner as in Working Example 3 to obtain the titled compound. Yield: 68%; melting point: 170-171° C. (THF-hexane).

¹H-NMR (DMSO-d₆) δ: 2.81 (2H, t, J=6.4 Hz), 3.54 (2H, q, J=6.4 Hz), 5.69 (2H, s), 7.08-7.28 (4H, m), 7.34-7.44 (2H, m), 7.60-7.69 (2H, m), 7.87-7.95 (2H, m), 8.16-8.21 (1H, m), 8.74 (1H, d, J=0.8 Hz), 8.96 (1H, t, J=6.4 Hz).

### Working Example 62

### 3-[2-(3-Fluorobenzyl)-2H-indazol-4-yl]-N-(2-hydroxyethyl)benzamide

3-[2-(3-Fluorobenzyl)-2H-indazol-4-yl]benzoic acid obtained in Reference Example 70 and 2-aminoethanol were used in the same manner as in Working Example 3 to obtain the titled compound. Yield: 46%; melting point: 187-188° C. (THF-hexane).

¹H-NMR (DMSO-d₆) δ: 3.36 (2H, q, J=6.0 Hz), 3.54 (2H, q, J=6.0 Hz), 4.73 (1H, t, J=6.0 Hz), 5.70 (2H, s), 7.08-7.28 (4H, m), 7.32-7.45 (2H, m), 7.55-7.70 (2, m), 7.83-7.93 (2H, m), 8.17 (1H, s), 8.55 (1H, t, J=6.0 Hz), 8.74 (1H, s).

### Working Example 63

### N-(2-Cyanoethyl)-3-[2-(4-fluorobenzyl)-2H-indazol-4-yl]benzamide

3-[2-(4-Fluorobenzyl)-2H-indazol-4-yl]benzoic acid obtained in Reference Example 73 and 3-aminopropanenitrile were used in the same manner as in Working Example 3 to obtain the titled compound. Yield: 74%; melting point: 133-134° C. (ethyl acetate-hexane).

¹H-NMR (DMSO-d₆) δ: 2.81 (2H, t, J=6.4 Hz), 3.54 (2H, q, J=6.4 Hz), 5.66 (2H, s), 7.11-7.28 (3H, m), 7.32-7.48 (3H, m), 7.59-7.69 (2H, m), 7.85-7.95 (2H, m), 8.17 (1H, t, J=1.5 Hz), 8.74 (1H, d, J=0.8 Hz), 8.96 (1H, t, J=6.4 Hz).

### Working Example 64

### 3-[2-(4-Fluorobenzyl)-2H-indazol-4-yl]-N-(2-hydroxyethyl)benzamide

3-[2-(4-Fluorobenzyl)-2H-indazol-4-yl]benzoic acid obtained in Reference Example 73 and 2-aminoethanol were used in the same manner as in Working Example 3 to obtain the titled compound. Yield: 66%; melting point: 137-138° C. (ethyl acetate-hexane).

¹H-NMR (DMSO-d₆) δ: 3.37 (2H, q, J=6.0 Hz), 3.54 (2H, q, J=6.0 Hz), 4.73 (1H, t, J=6.0 Hz), 5.66 (2H, s), 7.11-7.28 (3H, m), 7.31-7.48 (3H, m), 7.57-7.66 (2H, m), 7.83-7.93 (2H, m), 8.17 (1H, t, J=1.6 Hz), 8.55 (1H, t, J=6.0 Hz), 8.72 (1H, d, J=0.8 Hz).

### Working Example 65

### 3-[2-(3-Chloro-4-fluorobenzyl)-2H-indazol-4-yl]-N-(2-cyanoethyl)benzamide

3-[2-(3-Chloro-4-fluorobenzyl)-2H-indazol-4-yl]benzoic acid obtained in Reference Example 76 and 3-aminopropanenitrile were used in the same manner as in Working Example 3 to obtain the titled compound. Yield: 69%; melting point: 165-166° C. (ethyl acetate-hexane).

¹H-NMR (DMSO-d₆) δ: 2.21 (2H, t, J=6.4 Hz), 2.94 (2H, q, J=6.4 Hz), 5.07 (2H, s), 6.62-6.69 (1H, m), 6.73-6.83 (3H, m), 7.00-7.10 (3H, m), 7.26-7.36 (2H, m), 7.59 (1H, s), 8.15 (1H, s), 8.36 (1H, t, J=6.4 Hz).

### Working Example 66

### 3-[2-(3-Chloro-4-fluorobenzyl)-2H-indazol-4-yl]-N-(2-hydroxyethyl)benzamide

3-[2-(3-Chloro-4-fluorobenzyl)-2H-indazol-4-yl]benzoic acid obtained in Reference Example 76 and 2-aminoethanol were used in the same manner as in Working Example 3 to obtain the titled compound. Yield: 42%; melting point: 177-178° C. (ethyl acetate-hexane).

¹H-NMR (DMSO-d₆) δ: 3.37 (2H, q, J=6.0 Hz), 3.54 (2H, q, J=6.0 Hz), 4.72 (1H, t, J=6.0 Hz), 5.67 (2H, s), 7.22-7.27 (1H, m), 7.32-7.42 (3H, m), 7.56-7.69 (3H, m), 7.83-7.94 (2H, m), 8.17 (1H, s), 8.55 (1H, t, J=6.0 Hz), 8.75 (1H, s).

### Working Example 67

### N-(2-Cyanoethyl)-3-[2-(3-chlorobenzyl)-2H-indazol-4-yl]benzamide

3-[2-(3-Chlorobenzyl)-2H-indazol-4-yl]benzoic acid obtained in Reference Example 79 and 3-aminopropanenitrile were used in the same manner as in Working Example 3 to obtain the titled compound. Yield: 82%; melting point: 185-186° C. (THF-hexane).

¹H-NMR (DMSO-d₆) δ: 2.81 (2H, t, J=6.4 Hz), 3.54 (2H, q, J=6.4 Hz), 5.68 (2H, s), 7.22-7.46 (6H, m), 7.59-7.69 (2H, m), 7.86-7.95 (2H, m), 8.18 (1H, s), 8.75 (1H, s), 8.96 (1H, t, J=6.4 Hz).

### Working Example 68

### 3-[2-(3-Chlorobenzyl)-2H-indazol-4-yl]-N-(2-hydroxyethyl)benzamide

3-[2-(3-Chlorobenzyl)-2H-indazol-4-yl]benzoic acid obtained in Reference Example 79 and 2-aminoethanol were used in the same manner as in Working Example 3 to obtain the titled compound. Yield: 59%; melting point: 202-203° C. (THF-hexane).

¹H-NMR (DMSO-d₆) δ: 3.36 (2H, q, J=6.0 Hz), 3.54 (2H, q, J=6.0 Hz), 4.73 (1H, t, J=6.0 Hz), 5.69 (2H, s), 7.21-7.46 (6H, m), 7.57-7.68 (2H, m), 7.89 (2H, dd, J=8.1, 4.4 Hz), 8.17 (1H, s), 8.55 (1H, t, J=6.0 Hz), 8.75 (1H, s).

### Working Example 69

### N-(2-Cyanoethyl)-3-[2-(3,4-difluorobenzyl)-2H-indazol-4-yl]benzamide

3-[2-(3,4-Difluorobenzyl)-2H-indazol-4-yl]benzoic acid obtained in Reference Example 82 and 3-aminopropanenitrile were used in the same manner as in Working Example 3 to obtain the titled compound. Yield: 74%; melting point: 191-192° C. (THF-hexane).

¹H-NMR (DMSO-d₆) δ: 2.81 (2H, t, J=6.4 Hz), 3.54 (2H, q, J=6.4 Hz), 5.67 (2H, s), 7.17-7.29 (2H, m), 7.33-7.54 (3H, m), 7.58-7.69 (2H, m), 7.86-7.97 (2H, m), 8.18 (1H, t, J=1.6 Hz), 8.73 (1H, s), 8.96 (1H, t, J=6.4 Hz).

### Working Example 70

### 3-[2-(3,4-difluorobenzyl)-2H-indazol-4-yl]-N-(2-hydroxyethyl)benzamide

3-[2-(3,4-Difluorobenzyl)-2H-indazol-4-yl]benzoic acid obtained in Reference Example 82 and 2-aminoethanol were used in the same manner as in Working Example 3 to obtain the titled compound. Yield: 67%; melting point: 116-117° C. (THF-hexane).

¹H-NMR (DMSO-d₆) δ: 3.37 (2H, q, J=6.0 Hz), 3.54 (2H, q, J=6.0 Hz), 4.73 (1H, t, J=6.0 Hz ), 5.67 (2H, s), 7.17-7.29 (2H, m), 7.33-7.53 (3H, m), 7.57-7.68 (2H, m), 7.84-7.93 (2H, m), 8.17 (1H, t, J=1.6 Hz), 8.55 (1H, t, J=6.0 Hz), 8.74 (1H, d, J=0.8 Hz).

### Working Example 71

### 3-[2-(3-Chloro-5-fluorobenzyl)-2H-indazol-4-yl]-N-(2-cyanoethyl)benzamide

3-[2-(3-Chloro-5-fluorobenzyl)-2H-indazol-4-yl]benzoic acid obtained in Reference Example 85 and 3-aminopropanenitrile were used in the same manner as in Working Example 3 to obtain the titled compound. Yield: 70%; melting point: 207-208° C. (THF-hexane).

¹H-NMR (DMSO-d₆) δ: 2.81 (2H, t, J=6.4 Hz), 3.54 (2H, q, J=6.4 Hz), 5.70 (2H, s), 7.17-7.34 (3H, m), 7.35-7.44 (2H, m), 7.61-7.70 (2H, m), 7.87-7.97 (2H, m), (2H, m), 8.19 (1H, s), 8.76 (1H, s), 8.97 (1H, t, J=6.4 Hz).

### Working Example 72

### 3-[2-(3-Chloro-5-fluorobenzyl)-2H-indazol-4-yl]-N-(2-hydroxyethyl)benzamide

3-[2-(3-Chloro-5-fluorobenzyl)-2H-indazol-4-yl]benzoic acid obtained in Reference Example 85 and 2-aminoethanol were used in the same manner as in Working Example 3 to obtain the titled compound. Yield: 59%; melting point: 188-189° C. (THF-hexane).

¹H-NMR (DMSO-d₆) δ: 3.37 (2H, q, J=5.9 Hz), 3.54 (2H, q, J=5.9 Hz), 4.73 (1H, t, J=5.9 Hz), 5.70 (2H, s), 7.17-7.32 (3H, m), 7.33-7.43 (2H, m), 7.58-7.69 (2H, m), 7.86-7.93 (2H, m), 8.16-8.21 (1H, m), 8.56 (1H, t, J=5.9 Hz), 8.77 (1H, s).

### Working Example 73

### 3-(2-Benzyl-2H-indazol-4-yl)-N-(2-cyanoethyl)benzamide

3-(2-Benzyl-2H-indazol-4-yl]benzoic acid obtained in Reference Example 88 and 3-aminopropanenitrile were used in the same manner as in Working Example 3 to obtain the titled compound. Yield: 79%; melting point: 143-144° C. (THF-hexane).

¹H-NMR (DMSO-d₆) δ: 2.81 (2H, t, J=6.2 Hz), 3.53 (2H, q, J=6.2 Hz), 5.67 (2H, s), 7.20-7.41 (7H, m), 7.60-7.69 (2H, m), 7.86-7.94 (2H, m), 8.15-8.21 (1H, m), 8.71 (1H, s), 8.96 (1H, t, J=6.2 Hz).

### Working Example 74

### 3-(2-Benzyl-2H-indazol-4-yl)-N-(2-hydroxyethyl)benzamide

3-(2-Benzyl-2H-indazol-4-yl)benzoic acid obtained in Reference Example 88 and 2-aminoethanol were used in the same manner as in Working Example 3 to obtain the titled compound. Yield: 67%; melting point: 166-167° C. (THF-hexane).

¹H-NMR (DMSO-d₆) δ: 3.36 (2H, q, J=5.9 Hz), 3.54 (2H, q, J=5.9 Hz), 4.73 (1H, t, J=5.9 Hz), 5.67 (2H, s), 7.21-7.41 (7H, m), 7.57-7.66 (2H, m), 7.83-7.92 (2H, m), 8.13-8.19 (1H, m), 8.56 (1H, t, J=5.9 Hz), 8.72 (1H, s).

### Working Example 75

### N-(2-Cyanoethyl)-3-[2-[(6-methoxypyridin-2-yl)methyl]-2H-indazol-4-yl]benzamide

3-[2-[(6-Methoxypyridin-2-yl)methyl]-2H-indazol-4-yl]benzoic acid obtained in Reference Example 91 and 3-aminopropanenitrile were used in the same manner as in Working Example 3 to obtain the titled compound. Yield: 81%; melting point: 145-146° C. (THF-hexane).

¹H-NMR (DMSO-d₆) δ: 2.80 (2H, t, J=6.2 Hz), 3.53 (2H, q, J=6.2 Hz), 3.81 (3H, s), 5.70 (2H, s), 6.69 (1H, d, J=7.2 Hz), 6.74 (1H, d, J=8.3 Hz), 7.26 (1H, d, J=6.1 Hz), 7.38 (1H, dd, J=8.3, 7.2 Hz), 7.61-7.70 (3H, m), 7.86-7.95 (2H, m), 8.16-8.21 (1H, m), 8.73 (1H, s), 8.96 (1H, t, J=6.2 Hz).

### Working Example 76

### N-(2-Hydroxyethyl)-3-[2-[(6-methoxypyridin-2-yl)methyl]-2H-indazol-4-yl]benzamide

3-[2-[(6-Methoxypyridin-2-yl)methyl]-2H-indazol-4-yl]benzoic acid obtained in Reference Example 91 and 2-aminoethanol were used in the same manner as in Working Example 3 to obtain the titled compound. Yield: 60%; melting point: 104-105° C. (THF-hexane).

¹H-NMR (DMSO-d₆) δ: 3.36 (2H, q, J=6.2 Hz), 3.54 (2H, q, J=6.2 Hz), 3.81 (3H, s), 4.73 (1H, t, J=6.2 Hz), 5.70 (2H, s), 6.69 (1H, d, J=7.2 Hz), 6.74 (1H, d, J=8.0 Hz), 7.26 (1H, d, J=7.2 Hz), 7.33-7.42 (1H, m), 7.55-7.70 (3H, m), 7.83-7.94 (2H, m), 8.14-8.21 (1H, m), 8.55 (1H, t, J=6.2 Hz), 8.74 (1H, s).

### Working Example 77

### N-(2-Cyanoethyl)-6-[2-[3-(trifluoromethyl)benzyl]-2H-indazol-4-yl]pyridine-2-carboxamide

6-[2-[3-(Trifluoromethyl)benzyl]-2H-indazol-4-yl]pyridine-2-carboxylic acid obtained in Reference Example 93 and 3-aminopropanenitrile were used in the same manner as in Working Example 3 to obtain the titled compound. Yield: 38%; melting point: 178-179° C. (ethyl acetate-hexane).

¹H-NMR (DMSO-d₆) δ: 2.89 (2H, t, J=6.2 Hz), 3.63 (2H, q, J=6.2 Hz), 5.81 (2H, s), 7.42 (1H, dd, J=8.5,7.0 Hz), 7.55-7.63 (1H, m), 7.65-7.72 (2H, m), 7.73-7.87 (3H, m), 8.00-8.06 (1H, m), 8.14 (1H, t, J=7.0 Hz), 8.20-8.26 (1H, m), 9.00-9.09 (2H, m).

### Working Example 78

### N-(2-Hydroxyethyl)-6-[2-[3-(trifluoromethyl)benzyl]-2H-indazol-4-yl]pyridine-2-carboxamide

6-[2-[3-(Trifluoromethyl)benzyl]-2H-indazol-4-yl]pyridine-2-carboxylic acid obtained in Reference Example 93 and 2-aminoethanol were used in the same manner as in Working Example 3 to obtain the titled compound. Yield: 27%; melting point: 150-151° C. (ethyl acetate-hexane).

¹H-NMR (DMSO-d₆) δ: 3.48 (2H, q, J=5.9 Hz), 3.63 (2H, q, J=5.9 Hz); 4.98 (1H, t, J=5.9 Hz), 5.88 (2H, s), 7.42 (1H, dd, J=8.5, 7.0 Hz), 7.56-7.64 (1H, m), 7.64-7.73 (2H, m), 7.76 (1H, d, J=8.5 Hz), 7.80-7.87 (2H, m), 7.99-8.06 (1H, m), 8.13 (1H, t, J=7.0 Hz), 8.20-8.27 (1H, m), 8.64 (1H, t, J=5.9 Hz), 9.06 (1H, s).

### Working Example 79

### N-(2-Cyanoethyl)-2-[2-[3-(trifluoromethyl)benzyl]-2H-indazol-4-yl]pyridine-4-carboxamide

2-[2-[3-(Trifluoromethyl)benzyl]-2H-indazol-4-yl]pyridine-4-carboxylic acid obtained in Reference Example 95 and 3-aminopropanenitrile were used in the same manner as in Working Example 3 to obtain the titled compound. Yield: 28%; melting point: 147-148° C. (ethyl acetate-hexane).

¹H-NMR (DMSO-d₆) δ: 2.84 (2H, t, J=6.2 Hz), 3.58 (2H, q, J=6.2 Hz), 5.85 (2H, s), 7.38-7.48 (1H, m), 7.54-7.85 (7H, m), 8.42 (1H, s), 8.91 (1H, d, J=5.3 Hz), 9.13 (1H, s), 9.27 (1H, t, J=6.2 Hz).

### Working Example 80

### N-(2-Hydroxyethyl)-2-[2-[3-(trifluoromethyl)benzyl]-2H-indazol-4-yl]pyridine-2-carboxamide

2-[2-[3-(Trifluoromethyl)benzyl]-2H-indazol-4-yl]pyridine-4-carboxylic acid obtained in Reference Example 95 and 2-aminoethanol were used in the same manner as in Working Example 3 to obtain the titled compound. Yield: 55%; melting point: 50-51° C. (ethyl acetate-hexane).

¹H-NMR (DMSO-d₆) δ: 3.40 (2H, q, J=5.9 Hz), 3.56 (2H, q, J=5.9 Hz), 4.79 (1H, t, J=5.9 Hz), 5.85 (2H, s), 7.37-7.46 (1H, m), 7.55-7.86 (7H, m), 8.44 (1H, s), 8.83-8.93 (2H, m), 9.13 (1H, s).

### Working Example 81

### N-(2-Cyanoethyl)-5-[2-[3-(trifluoromethyl)benzyl]-2H-indazol-4-yl]pyridine-3-carboxamide

5-[2-[3-(Trifluoromethyl)benzyl]-2H-indazol-4-yl]pyridine-3-carboxylic acid obtained in Reference Example 97 and 3-aminopropanenitrile were used in the same manner as in Working Example 3 to obtain the titled compound. Yield: 75%; melting point: 192-193° C. (THF-hexane).

¹H-NMR (DMSO-d₆) δ: 2.82 (2H, t, J=6.2 Hz), 3.57 (2H, q, J=6.2 Hz), 5.79 (2H, s), 7.31-7.46 (2H, m), 7.54-7.75 (4H, m), 7.81 (1H, s), 8.51 (1H, t, J=2.3 Hz), 8.90 (1H, s), 9.05 (1H, d, J=1.9 Hz), 9.12 (1H, d, J=1.9 Hz), 9.16 (1H, t, J=6.2 Hz).

### Working Example 82

### N-(2-Hydroxyethyl)-5-[2-[3-(trifluoromethyl)benzyl]-2H-indazol-4-yl]pyridine-3-carboxamide

5-[2-[3-(Trifluoromethyl)benzyl]-2H-indazol-4-yl]pyridine-3-carboxylic acid obtained in Reference Example 97 and 2-aminoethanol were used in the same manner as in Working Example 3 to obtain the titled compound. Yield: 34%; melting point: 185-186° C. (THF-hexane).

¹H-NMR (DMSO-d₆) δ: 3.39 (2H, q, J=5.9 Hz), 3.56 (2H, q, J=5.9 Hz), 4.77 (1H, t, J=5.9 Hz), 5.79 (2H, s), 7.30-7.45 (2H, m), 7.54-7.74 (4H, m), 7.81 (1H, s), 8.51 (1H, t, J=2.1 Hz), 8.77 (1H, t, J=5.9 Hz), 8.91 (1H, s), 9.04 (1H, d, J=1.9 Hz), 9.08 (1H, d, J=1.9 Hz).

### Working Example 83

### N-(2-Cyanoethyl)-4-[2-[3-(trifluoromethyl)benzyl]-2H-indazol-4-yl]pyridine-2-carboxamide

4-[2-[3-(Trifluoromethyl)benzyl]-2H-indazol-4-yl]pyridine-2-carboxylic acid obtained in Reference Example 99 and 3-aminopropanenitrile were used in the same manner as in Working Example 3 to obtain the titled compound. Yield: 81%; melting point: 166-167° C. (THF-hexane).

¹H-NMR (DMSO-d₆) δ: 2.85 (2H, t, J=6.2 Hz), 3.60 (2H, q, J=6.2 Hz), 5.82 (2H, s), 7.37-7.46 (2H, m), 7.55-7.72 (3H, m), 7.73-7.82 (2H, m), 7.99 (1H, dd, J=4.9, 1.9 Hz), 8.34 (1H, d, J=1.9 Hz), 8.79 (1H, d, J=4.9 Hz), 8.90(1H, s), 9.24 (1H, t, J=6.2 Hz).

### Working Example 84

### N-(2-Hydroxyethyl)-4-[2-[3-(trifluoromethyl)benzyl]-2H-indazol-4-yl]pyridine-2-carboxamide

4-[2-[3-(Trifluoromethyl)benzyl]-2H-indazol-4-yl]pyridine-2-carboxylic acid obtained in Reference Example 99 and 2-aminoethanol were used in the same manner as in Working Example 3 to obtain the titled compound. Yield: 68%; melting point: 131-132° C. (ethyl acetate-hexane).

¹H-NMR (DMSO-d₆) δ: 3.43 (2H, q, J=5.8 Hz), 3.57 (2H, q, J=5.8 Hz), 4.83 (1H, t, J=5.8 Hz), 5.82 (2H, s), 7.38-7.46 (2H, m), 7.55-7.72 (3H, m), 7.72-7.82 (2H, m), 7.97 (1H, dd, J=4.9, 1.9 Hz), 8.33 (1H, s), 8.73-8.82 (2H, m), 8.90 (1H, s).

### Working Example 85

### N-(2-Cyanoethyl)-3-[2-[3-(trifluoromethyl)benzyl]-1,3-benzoxazol-4-yl]benzamide

3-[2-[3-(Trifluoromethyl)benzyl]-1,3-benzoxazol-4-yl]benzoic acid obtained in Reference Example 102 and 3-aminopropanenitrile were used in the same manner as in Working Example 3 to obtain the titled compound. Yield: 66%; melting point: 117-118° C. (ethyl acetate-hexane).

¹H-NMR (CDCl₃) δ: 2.77 (2H, t, J=6.3 Hz), 3.74 (2H, q, J=6.3 Hz), 4.38 (2H, s), 6.77 (1H, br s), 7.35-7.62 (7H, m), 7.68 (1H, s), 7.81 (1H, d, J=7.8 Hz), 8.11 (1H, d, J=7.8 Hz), 8.37 (1H, t, J=1.8 Hz).

### Working Example 86

### N-(2-Hydroxyethyl)-3-[2-[3-(trifluoromethyl)benzyl]-1,3-benzoxazol-4-yl]benzamide

3-[2-[3-(Trifluoromethyl)benzyl]-1,3-benzoxazol-4-yl]benzoic acid obtained in Reference Example 102 and 2-aminoethanol were used in the same manner as in Working Example 22 to obtain the titled compound. Yield: 47%; melting point: 132-133° C. (ethyl acetate-hexane).

¹H-NMR (CDCl₃) δ: 2.70-2.79 (1H, m), 3.64 (2H, q, J=5.1 Hz), 3.84 (2H, q, J=5.1 Hz), 4.37 (2H, s), 6.82 (1H, br s), 7.34-7.61 (7H, m), 7.68 (1H, s), 7.79 (1H, dt, J=7.5, 1.5 Hz), 8.07 (1H, dt, J=7.5, 1.5 Hz), 8.31 (1H, t, J=1.5 Hz).

### Working Example 87

### N-(2-Hydroxyethyl)-3-[2-[3-(trifluoromethyl)benzyl]-1,3-benzoxazol-7-yl]benzamide

3-[2-[3-(Trifluoromethyl)benzyl]-1,3-benzoxazol-7-yl]benzoic acid obtained in Reference Example 104 and 2-aminoethanol were used in the same manner as in Working Example 22 to obtain the titled compound. Yield: 59%; melting point: 122-123° C. (ethyl acetate-hexane).

¹H-NMR (CDCl₃) δ: 2.43 (1H, br s), 3.68 (2H, q, J=5.1 Hz), 3.87 (2H, q, J=5.1 Hz), 4.38 (2H, s), 6.62 (1H, br s), 7.36-7.63 (6H, m), 7.66-7.71 (2H, m), 7.78 (1H, d, J=8.4 Hz), 7.93 (1H, d, J=7.8 Hz), 8.21 (1H, s).

### Working Example 88

### N-(2-Cyanoethyl)-3-[1-methyl-2-[3-(trifluoromethyl)benzyl]-1H-benzimidazol-4-yl]benzamide

3-[1-Methyl-2-[3-(trifluoromethyl)benzyl]-1H-benzimidazol-4-yl]benzoic acid obtained in Reference Example 109 and 3-aminopropanenitrile were used in the same manner as in Working Example 3 to obtain the titled compound. Yield: 40%; melting point: 168-169° C. (ethyl acetate-hexane).

¹H-NMR (DMSO-d₆) δ: 2.73 (2H, t, J=6.3 Hz), 3.63 (3H, s), 3.69 (2H, q, J=6.3 Hz), 4.42 (2H, s), 7.20-7.62 (9H, m), 7.82 (1H, dd, J=6.3, 1.5 Hz), 8.15 (1H, dt, J=6.3, 1.5 Hz), 8.44 (1H, s).

### Working Example 89

### N-(2-Hydroxyethyl)-3-[1-methyl-2-[3-(trifluoromethyl)benzyl]-1H-benzimidazol-4-yl]benzamide

3-[1-Methyl-2-[3-(trifluoromethyl)benzyl]-1H-benzimidazol-4-yl]benzoic acid obtained in Reference Example 109 and 2-aminoethanol were used in the same manner as in Working Example 3 to obtain the titled compound. Yield: 45%; melting point: 147-148° C. (ethyl acetate-hexane).

¹H-NMR (DMSO-d₆) δ: 3.30-3.42 (2H, m), 3.45-3.61 (2H, m), 3.76 (3H, s), 4.49 (2H, s), 4.74 (1H, br s), 7.34 (1H, t, J=7.5 Hz), 7.47-7.62 (6H, m), 7.75 (1H, s), 7.81 (1H, d, J=7.5 Hz), 8.31-8.38 (2H, m), 8.48-8.52 (1H, m).

### Working Example 90

### N-(2-Cyanoethyl)-3-[2-[3-(trifluoromethyl)benzyl]imidazo[1,2-a]pyridin-8-yl]benzamide

3-[2-[3-(Trifluoromethyl)benzyl]imidazo[1,2-a]pyridin-8-yl]benzoic acid obtained in Reference Example 111 and 3-aminopropanenitrile were used in the same manner as in Working Example 3 to obtain the titled compound. Yield: 64%; melting point: 126-127° C. (ethyl acetate-hexane).

¹H-NMR (CDCl₃) δ: 2.73 (2H, t, J=6.6 Hz), 3.69 (2H, q, J=6.6 Hz), 4.23 (2H, s), 6.87 (1H, t, J=6.9 Hz), 7.22-7.26 (1H, m), 7.30 (1H, d, J=6.6 Hz), 7.40-7.60 (5H, m), 7.80-7.92 (2H, m), 7.98 (1H, dt, J=7.8, 1.5 Hz), 8.03 (1H, dd, J=6.9, 1.2 Hz), 8.43 (1H, t, J=1.5 Hz).

### Working Example 91

### N-(2-Hydroxyethyl)-3-[2-[3-(trifluoromethyl)benzyl]imidazo[1,2-a]pyridin-8-yl]benzamide

3-[2-[3-(Trifluoromethyl)benzyl]imidazo[1,2-a]pyridin-8-yl]benzoic acid obtained in Reference Example 111 and 2-aminoethanol were used in the same manner as in Working Example 3 to obtain the titled compound. Yield: 61%; melting point: 136-137° C. (ethyl acetate-hexane).

¹H-NMR (CDCl₃) δ: 2.20 (1H, br s), 3.47 (2H, t, J=4.8 Hz), 3.63 (2H, q, J=4.8 Hz), 4.21 (2H, s), 6.84 (1H, t, J=6.6 Hz), 7.21-7.26 (2H, m), 7.36-7.57 (5H, m), 7.78 (1H, d, J=7.5 Hz), 7.83 (1H, br s), 7.89 (1H, d, J=8.1 Hz), 8.00 (1H, dd, J=6.9, 1.2 Hz), 8.30 (1H, s).

### Working Example 92

### N-(2-Cyanoethyl)-3-[2-[3-(trifluoromethyl)benzyl]pyrazolo[1,5-a]pyrimidin-7-yl]benzamide

3-[2-[3-(Trifluoromethyl)benzyl]pyrazolo[1,5-a]pyrimidin-7-yl]benzoic acid obtained in Reference Example 117 and 3-aminopropanenitrile were used in the same manner as in Working Example 3 to obtain the titled compound. Yield: 87%; melting point: 153-154° C. (ethyl acetate-hexane).

¹H-NMR (CDCl₃) δ: 2.78 (2H, t, J=6.2 Hz), 3.76 (2H, q, J=6.2 Hz), 4.27 (2H, s), 6.54 (1H, s), 6.68 (1H, br s), 6.91 (1H, d, J=4.3 Hz), 7.40-7.47.(1H, m), 7.48-7.54 (2H, m), 7.61 (1H, s), 7.68 (1H, t, J=7.9 Hz), 7.97 (1H, d, J=8.1 Hz), 8.27 (1H, d, J=8.3 Hz), 8.48-8.52 (2H, m).

### Working Example 93

### N-(2-Hydroxyethyl)-3-[2-[3-(trifluoromethyl)benzyl]pyrazolo[1,5-a]pyrimidin-7-yl]benzamide

2-Aminoethanol (7.56 μL, 0.18 mmol) was added to a methanol (1 mL) solution of 3-[2-[3-(trifluoromethyl)benzyl]pyrazolo[1,5-a]pyrimidin-7-yl]benzoic acid (60 mg, 0.15 mmol) obtained in Reference Example 117 and DMTMM (53.1 mg, 0.18 mmol), and the mixture was stirred for 5 hours at room temperature. The reaction solution was concentrated at reduced pressure, and the addition of saturated sodium bicarbonate aqueous solution was followed by extraction with ethyl acetate. The organic layer was dried over anhydrous magnesium sulfate and then concentrated at reduced pressure. The residue was purified by silica gel column chromatography (ethyl acetate-methanol 100:0→90:10) and was recrystallized from ethyl acetate-hexane to give 30 mg of the titled compound (yield 45%) in the form of crystals. Melting point: 127-128° C.

¹H-NMR (CDCl₃) δ: 2.43 (1H, br s), 3.66 (2H, q, J=5.4 Hz), 3.85 (2H, br s), 4.26 (2H, s), 6.53 (1H, s), 6.71 (1H, br s), 6.90 (1H, d, J=4.5 Hz), 7.44 (1H, d, J=7.6 Hz), 7.47-7.54 (2H, m), 7.61 (1H, s), 7.65 (1H, t, J=8.0 Hz), 7.96 (1H, d, J=8.0 Hz), 8.24 (1H, d, J=8.0 Hz), 8.47 (1H, s), 8.49 (1H, d, J=4.2 Hz).

### Working Example 94

### N-(2-Cyanoethyl)-3-(2-[[3-(trifluoromethyl)phenyl]amino][1,2,4]triazolo[1,5-a]pyridin-8-yl)benzamide

A DMF (1.6 mL) solution of 3-(2-[[3-(trifluoromethyl)phenyl]amino][1,2,4]triazolo[1,5-a]pyridin-8-yl)benzoic acid (80 mg, 0.201 mmol) obtained in Reference Example 122, 3-aminopropanenitrile (0.016 mL, 0.221 mmol), HATU (91.6 mg, 0.241 mmol), and N,N-diisopropylethylamine (0.042 mL, 0.241 mmol) was stirred for 3 hours at room temperature. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate) and recrystallized from hexane and ethyl acetate to give 68.4 mg of the titled compound (yield 76%) in solid form. Melting point: 214-215° C.

¹H-NMR (DMSO-d₆) δ: 2.81 (2H, t, J=6.5 Hz), 3.55 (2H, q, J=6.5 Hz), 7.15-7.26 (2H, m), 7.53 (1H, t, J=8.0 Hz), 7.65 (1H, t, J=8.0 Hz), 7.89-7.97 (3H, m), 8.21 (1H, br s), 8.34-8.41 (1H, m), 8.49 (1H, t, J=1.6 Hz), 8.89 (1H, dd, J=6.7, 1.0 Hz), 8.99 (1H, t, J=5.7 Hz), 10.22 (1H, s).

### Working Example 95

### N-(2-Hydroxyethyl)-3-(2-[[3-(trifluoromethyl)phenyl]amino][1,2,4]triazolo[1,5-a]pyridin-8-yl)benzamide

3-(2-[[3-(Trifluoromethyl)phenyl]amino][1,2,4]triazolo[1,5-a]pyridin-8-yl)benzoic acid obtained in Reference Example 122 and 2-aminoethanol were used in the same manner as in Working Example 94 to obtain the titled compound. Yield: 69%; melting point: 215-216° C. (ethyl acetate-hexane).

¹H-NMR (DMSO-d₆) δ: 3.38 (2H, q, J=5.9 Hz), 3.55 (2H, q, J=5.9 Hz), 4.76 (1H, t, J=5.9 Hz), 7.16-7.26 (2H, m), 7.48-7.67 (2H, m), 7.89-7.99 (3H, m), 8.21 (1H, s), 8.35 (1H, d, J=8.0 Hz), 8.48 (1H, s), 8.59 (1H, t, J=5.9 Hz), 8.88 (1H, d, J=5.7 Hz), 10.22 (1H, s).

### Working Example 96

### N-(2-Cyanoethyl)-3-[1-oxo-2-[3-(trifluoromethyl)benzyl]-2,3-dihydro-1H-isoindol-4-yl]benzamide

3-[1-Oxo-2-[3-(trifluoromethyl)benzyl]-2,3-dihydro-1H-isoindol-4-yl]benzoic acid obtained in Reference Example 124 and 3-aminopropanenitrile were used in the same manner as in Working Example 3 to obtain the titled compound. Yield: 63%; melting point: 191-192° C. (ethyl acetate-hexane).

¹H-NMR (CDCl₃) δ: 2.78 (2H, t, J=6.3 Hz), 3.75 (2H, q, J=6.3 Hz), 4.29 (2H, s), 4.79 (2H, s), 7.02 (1H, br s), 7.38-7.65 (8H, m), 7.76-7.93 (3H, m).

### Working Example 97

### N-(2-Hydroxyethyl)-3-[1-oxo-2-[3-(trifluoromethyl)benzyl]-2,3-dihydro-1H-isoindol-4-yl]benzamide

3-[1-Oxo-2-[3-(trifluoromethyl)benzyl]-2,3-dihydro-1H-isoindol-4-yl]benzoic acid obtained in Reference Example 124 and 2-aminoethanol were used in the same manner as in Working Example 3 to obtain the titled compound. Yield: 67%; melting point: 148-149° C. (ethyl acetate-hexane).

¹H-NMR (CDCl₃) δ: 2.75 (1H, br s), 3.67 (2H, q, J=4.8 Hz), 3.81 (2H, t, J=4.5 Hz), 4.30 (2H, s), 4.79 (2H, s), 6.90 (1H, br s), 7.37-7.60 (8H, m), 7.77-7.90 (3H, m).

### Working Example 98

### N-(2-Cyanoethyl)-3-[3-oxo-2-[3-(trifluoromethyl)benzyl]-2,3-dihydro[1,2,4]triazolo[4,3-a]pyridin-8-yl]benzamide

Ethyl 3-[3-oxo-2-[3-(trifluoromethyl)benzyl]-2,3-dihydro[1,2,4]triazolo[4,3-a]pyridin-8-yl]benzoate obtained in Reference Example 128 and 3-aminopropanenitrile were used in the same manner as in Working Example 1 to obtain the titled compound. Yield: 82%, melting point: 217-219° C.

¹H-NMR (CDCl₃) δ: 2.77 (2H, t, J=6.0 Hz), 3.74 (2H, q, J=6.0 Hz), 5.26 (2H, s), 6.64 (2H, t, J=6.6 Hz), 7.23 (1H, d, J=6.9 Hz), 7.47 (1H, t, J=4.5 Hz), 7.50-7.65 (3H, m), 7.71 (1H, s), 7.82 (2H, d, J=6.9 Hz), 8.04 (1H, d, J=7.5 Hz), 8.29 (1H, s).

### Working Example 99

### N-(2-Hydroxyethyl)-3-[3-oxo-2-[3-(trifluoromethyl)benzyl]-2,3-dihydro[1,2,4]triazolo[4,3-a]pyridin-8-yl]benzamide

Ethyl 3-[3-oxo-2-[3-(trifluoromethyl)benzyl]-2,3-dihydro[1,2,4]triazolo[4,3-a]pyridin-8-yl]benzoate obtained in Reference Example 128 and 2-aminoethanol were used in the same manner as in Working Example 1 to obtain the titled compound. Yield: 75%, melting point: 148-150° C.

¹H-NMR (CDCl₃) δ: 2.55 (1H, br s), 3.64 (2H, q, J=4.6 Hz), 3.84 (2H, t, J=4.6 Hz), 5.25 (2H, s), 6.62 (1H, t, J=7.1 Hz), 6.65-6.80 (1H, m), 7.30 (1H, d, J=6.9 Hz), 7.46 (1H, t, J=7.8 Hz), 7.50-7.60 (3H, m), 7.62 (1H, d, J=8.1 Hz), 7.80 (2H, d, J=6.9 Hz), 8.02 (1H, d, J=7.8 Hz), 8.25 (1H, s).

### Working Example 100

### N-Cyclopropyl-3-[3-oxo-2-[3-(trifluoromethyl)benzyl]-2,3-dihydro[1,2,4]triazolo[4,3-a]pyridin-8-yl]benzamide

3-[3-Oxo-2-[3-(trifluoromethyl)benzyl]-2,3-dihydro[1,2,4]triazolo[4,3-a]pyridin-8-yl]benzoic acid obtained in Reference Example 129 and cyclopropylamine were used in the same manner as in Working Example 3 to obtain the titled compound. Yield: 94%, melting point: 157-158° C.

¹H-NMR (CDCl₃) δ: 0.55-0.65 (2H, m), 0.80-1.00 (2H, m), 2.85-3.00 (1H, m), 5.25 (2H, s), 6.26 (1H, br s), 6.64 (1H, t, J=6.9 Hz), 7.31 (1H, d, J=6.9 Hz), 7.20-7.65 (4H, m), 7.69 (1H, s), 7.75 (1H, d, J=7.5 Hz), 7.81 (1H, d, J=6.9 Hz), 8.01 (1H, d, J=7.5 Hz), 8.20 (1H, s).

### Working Example 101

### N-(3-Hydroxypropyl)-3-[3-oxo-2-[3-(trifluoromethyl)benzyl]-2,3-dihydro[1,2,4]triazolo[4,3-a]pyridin-8-yl]benzamide

3-[3-Oxo-2-[3-(trifluoromethyl)benzyl]-2,3-dihydro[1,2,4]triazolo[4,3-a]pyridin-8-yl]benzoic acid obtained in Reference Example 129 and 4-amino-1-butyl alcohol were used in the same manner as in Working Example 3 to obtain the titled compound. Yield: 91%, melting point: 148-149° C.

¹H-NMR (CDCl₃) δ: 1.75-1.90 (2H, m), 2.95 (1H, br s), 3.65 (2H, q, J=6.1 Hz), 3.72 (2H, t, J=5.6 Hz), 5.25 (2H, s), 6.64 (1H, t, J=6.9 Hz), 6.70-6.80 (1H, br s), 7.31 (1H, d, J=6.6 Hz), 7.40-7.65 (4H, m), 7.70 (1H, s), 7.80 (2H, t, J=6.6 Hz), 8.03 (1H, d, J=7.5 Hz), 8.24 (1H, s).

Table 1 shows the structures of the compounds obtained in Working Examples 1 through 101.

TABLE 1

Working Example 1

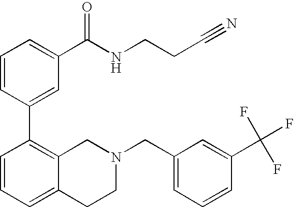

Working Example 2

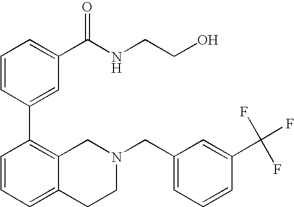

Working Example 3

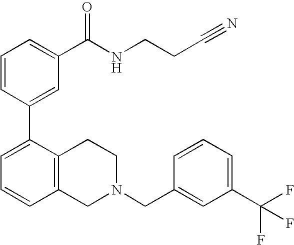

Working Example 4

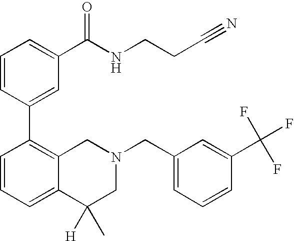

Working Example 5

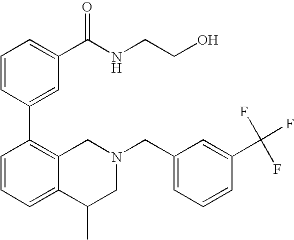

Working Example 6

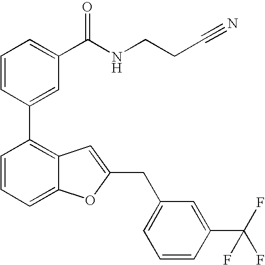

Working Example 7

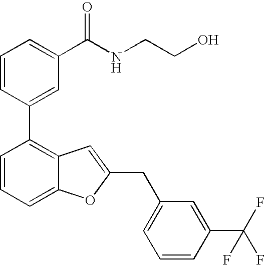

Working Example 8

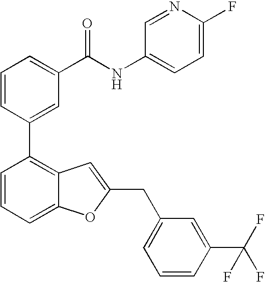

Working Example 9

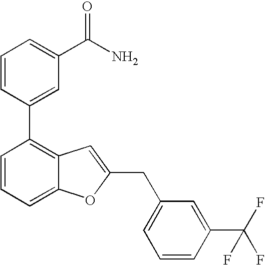

Working Example 10

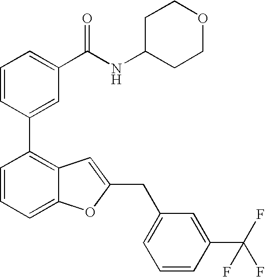

Working Example 11

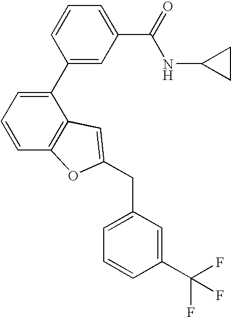

Working Example 12

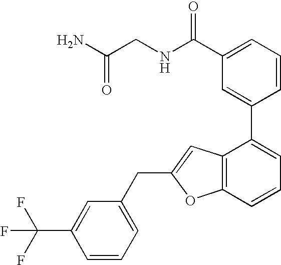

Working Example 13

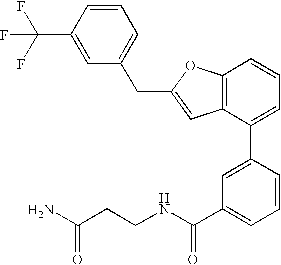

Working Example 14

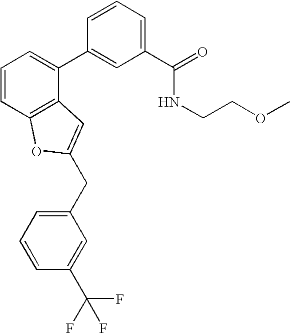

Working Example 15

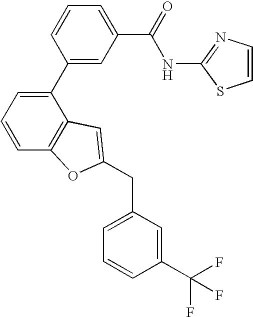

Working Example 16

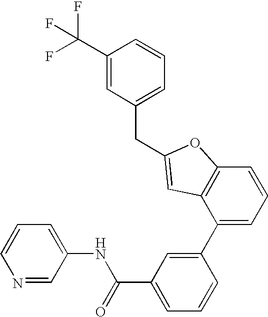

Working Example 17

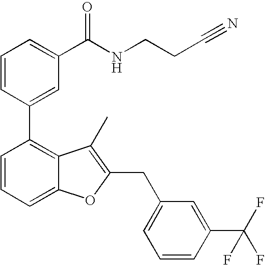

Working Example 18

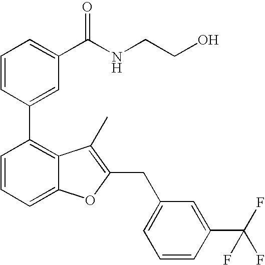

Working Example 19

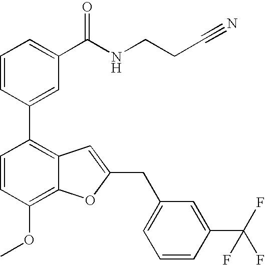

Working Example 20

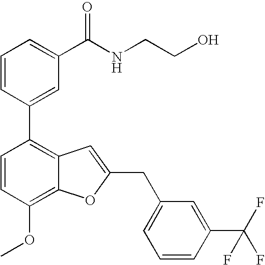

Working Example 21

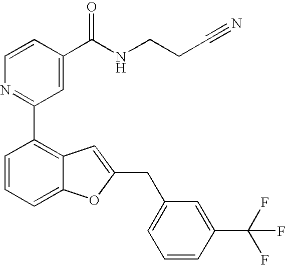

Working Example 22

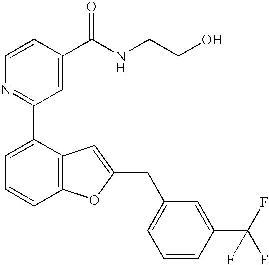

Working Example 23

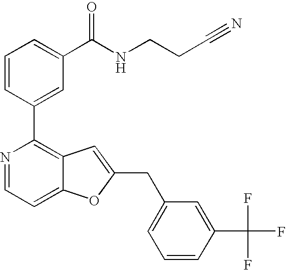

Working Example 24

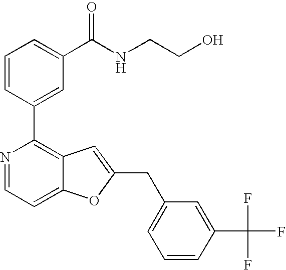

Working Example 25

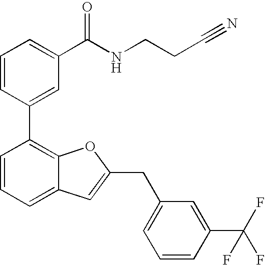

Working Example 26

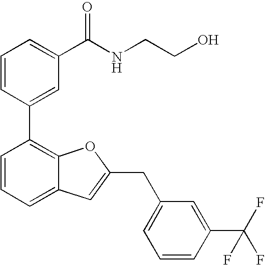

Working Example 27

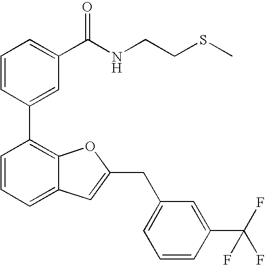

Working Example 28

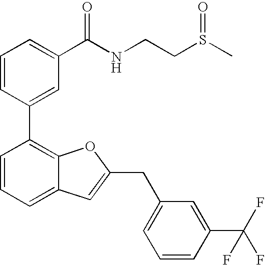

Working Example 29

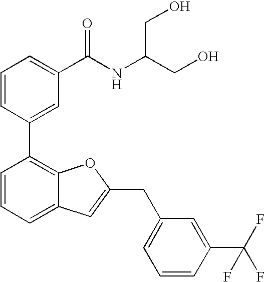

Working Example 30

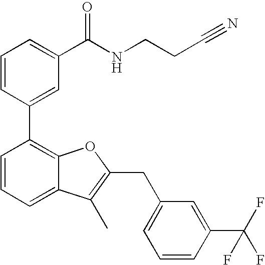

Working Example 31

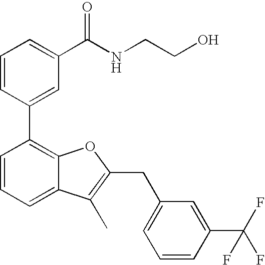

Working Example 32

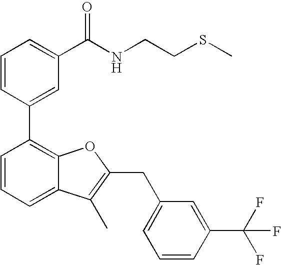

Working Example 33

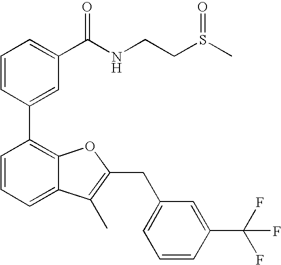

Working Example 34

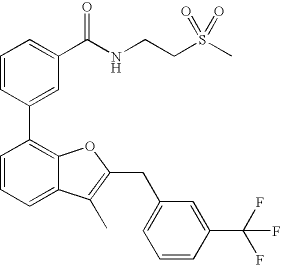

Working Example 35

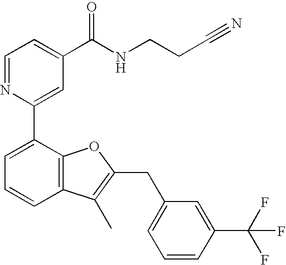

Working Example 36

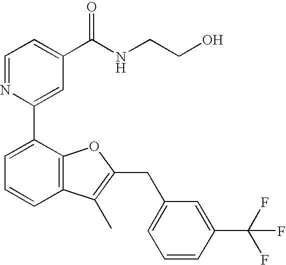

Working Example 37

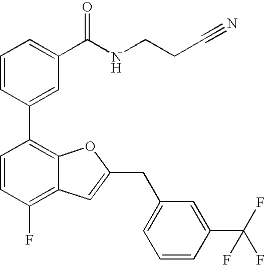

Working Example 38

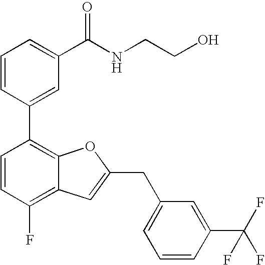

Working Example 39

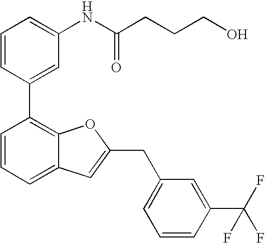

Working Example 40

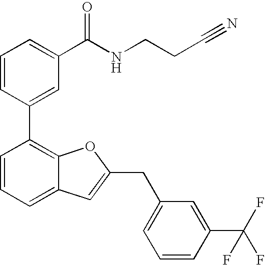

Working Example 41

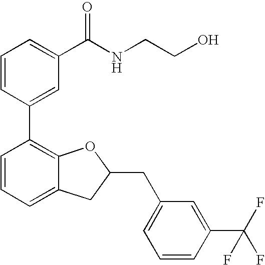

Working Example 42

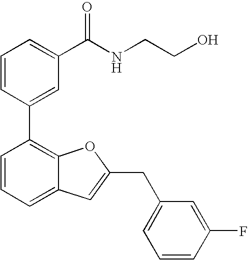

Working Example 43

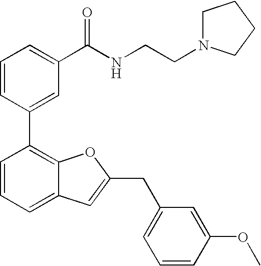

Working Example 44

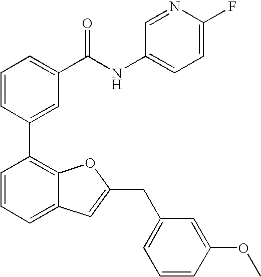

Working Example 45

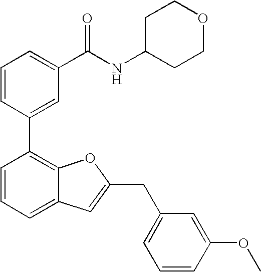

Working Example 46

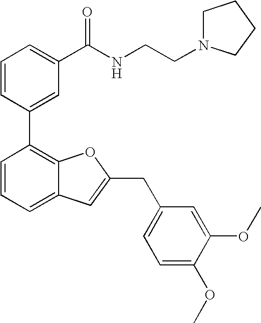

Working Example 47

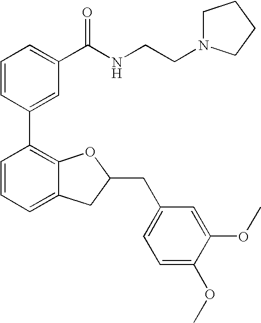

Working Example 48

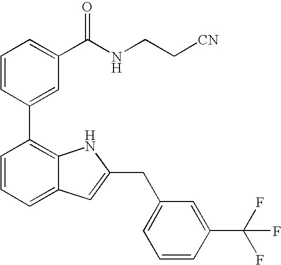

Working Example 49

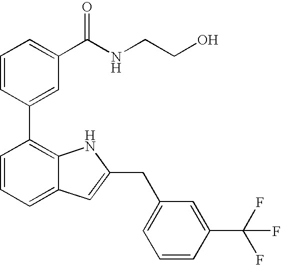

Working Example 50

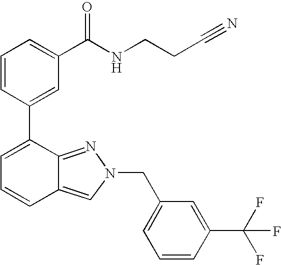

Working Example 51

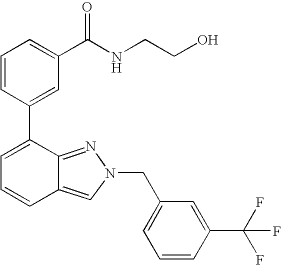

Working Example 52

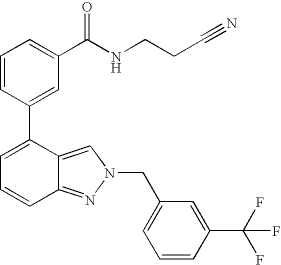

Working Example 53

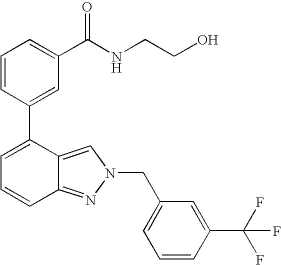

Working Example 54

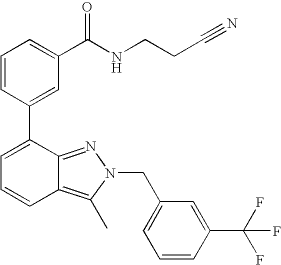

Working Example 55

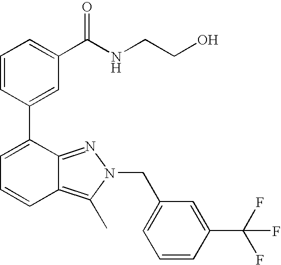

Working Example 56

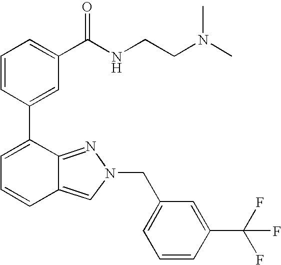

Working Example 57

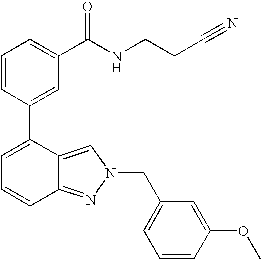

Working Example 58

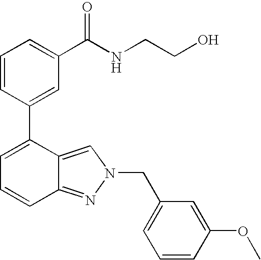

Working Example 59

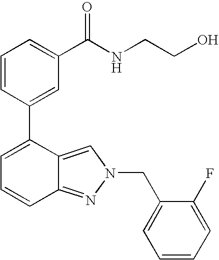

Working Example 60

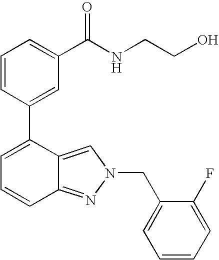

Working Example 61

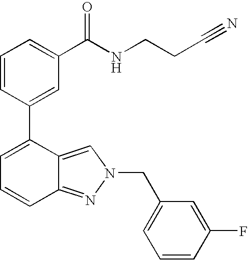

Working Example 62

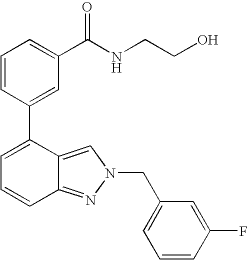

Working Example 63

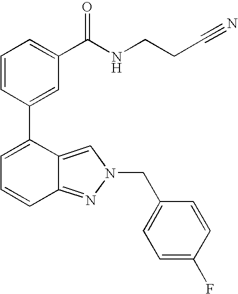

Working Example 64

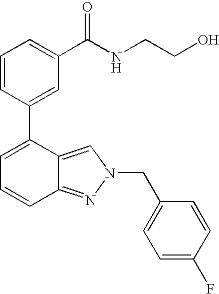

Working Example 65

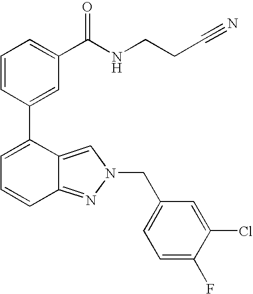

Working Example 66

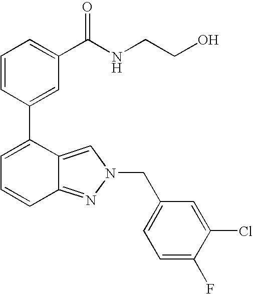

Working Example 67

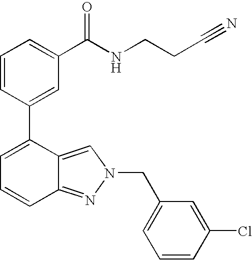

Working Example 68

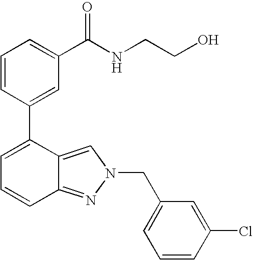

Working Example 69

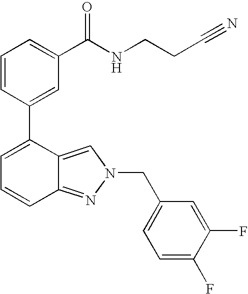

Working Example 70

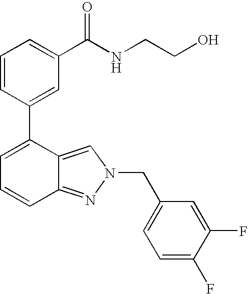

Working Example 71

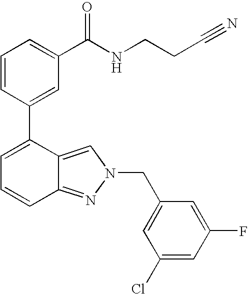

Working Example 72

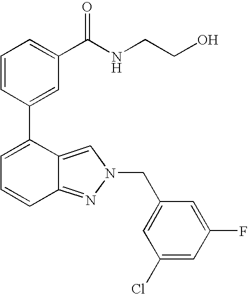

Working Example 73

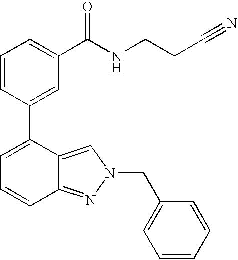

Working Example 74

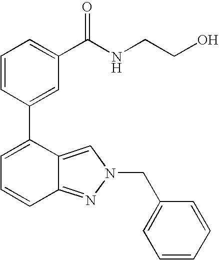

Working Example 75

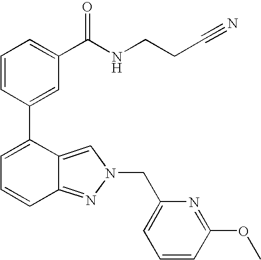

Working Example 76

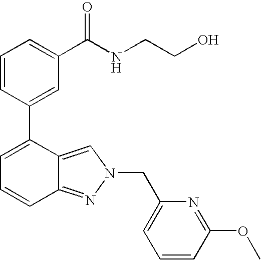

Working Example 77

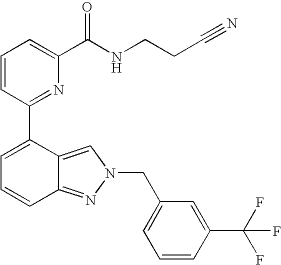

Working Example 78

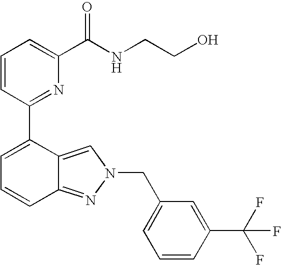

Working Example 79

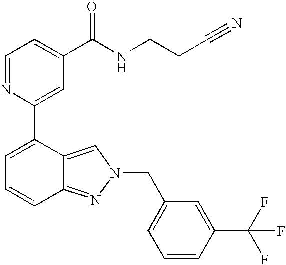

Working Example 80

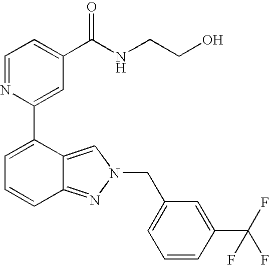

Working Example 81

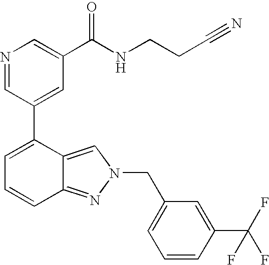

Working Example 82

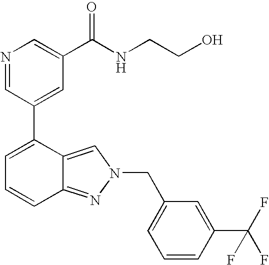

Working Example 83

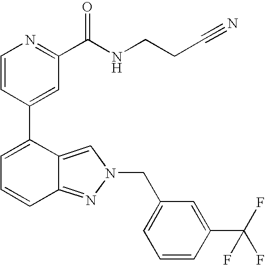

Working Example 84

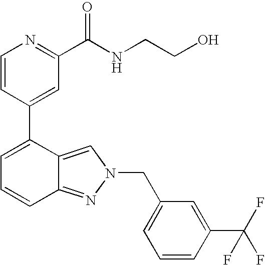

Working Example 85

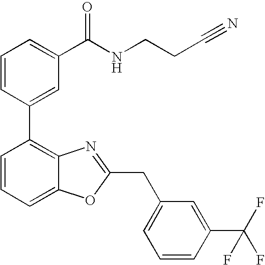

Working Example 86

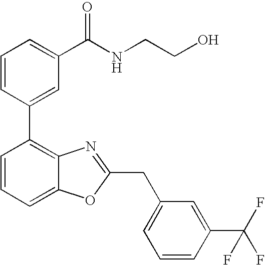

Working Example 87

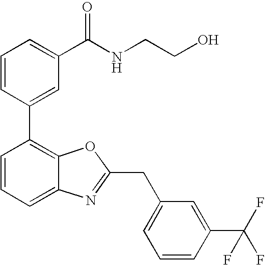

Working Example 88

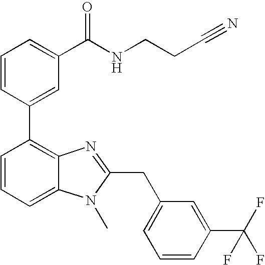

Working Example 89

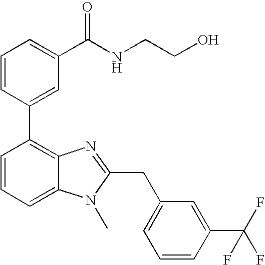

Working Example 90

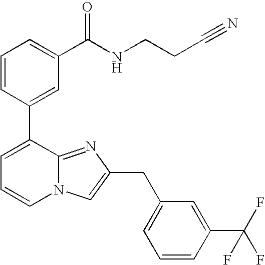

Working Example 91

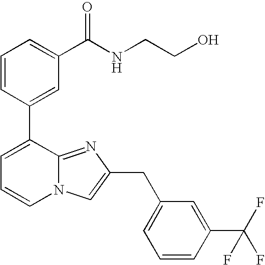

Working Example 92

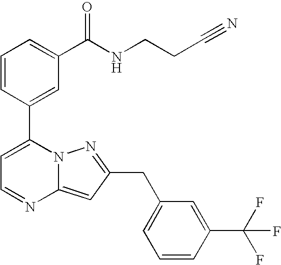

Working Example 93

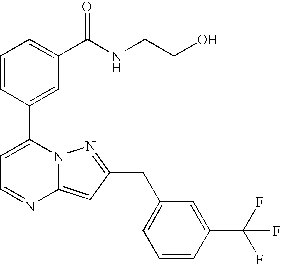

Working Example 94

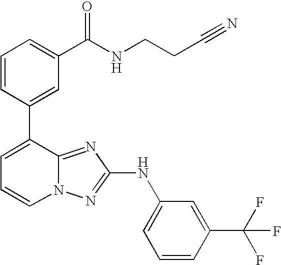

Working Example 95

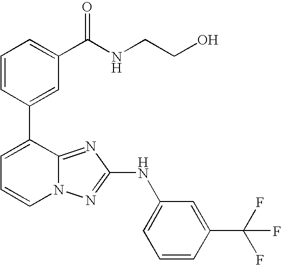

Working Example 96

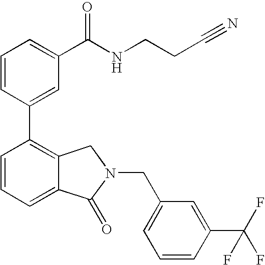

Working Example 97

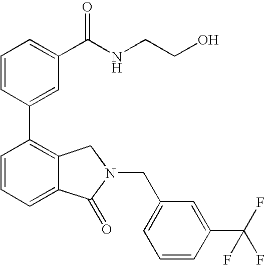

Working Example 98

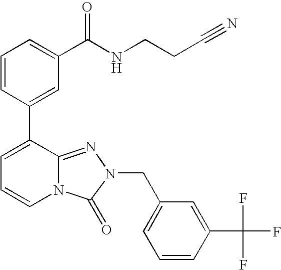

Working Example 99

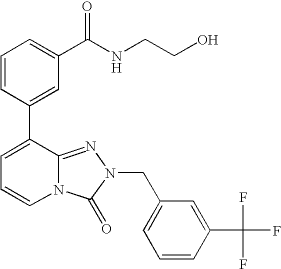

Working Example 100

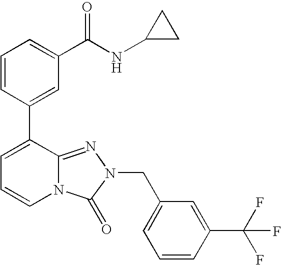

Working Example 101

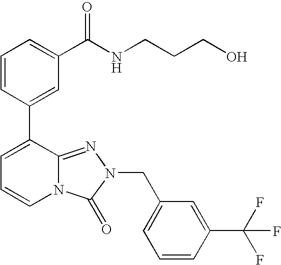

### Working Examples 102-141

The compounds of Working Examples 102 through 141 were synthesized by reactions between various amines and 3-[2-[3-(trifluoromethyl)benzyl]-1-benzofuran-4-yl]benzoic acid obtained in Reference Example 10 in the same manner as in Working Example 3. The synthesized compounds are shown in Table 2.

TABLE 2

| Working | | |
|---|---|---|
| Example | | |
| No. | R | LCMS |
| 102 | 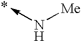 | 410 |
| 103 | 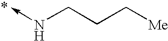 | 452 |
| 104 | 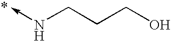 | 454 |
| 105 | 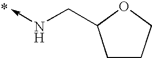 | 480 |
| 106 | 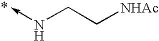 | 481 |
| 107 | 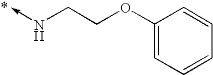 | 514 |
| 108 | 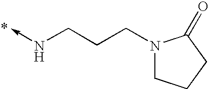 | 521 |
| 109 | 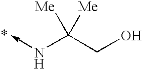 | 468 |
| 110 | 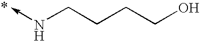 | 468 |
| 111 | 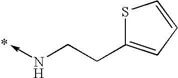 | 506 |
| 112 | 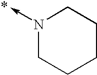 | 464 |
| 113 | 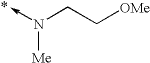 | 468 |
| 114 | 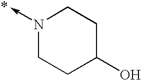 | 480 |
| 115 | 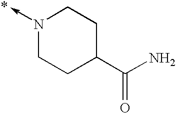 | 507 |
| 116 | 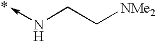 | 467 |
| 117 | 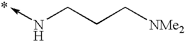 | 481 |
| 118 | 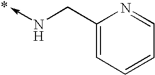 | 487 |
| 119 | 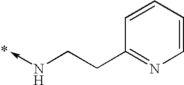 | 501 |
| 120 | 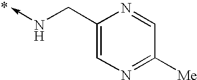 | 502 |
| 121 | 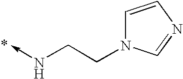 | 504 |
| 122 | 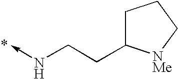 | 507 |
| 123 | 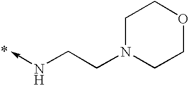 | 509 |
| 124 | 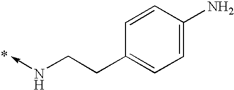 | 513 |
| 125 | 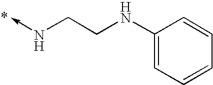 | 515 |
| 126 | 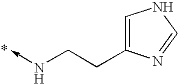 | 490 |
| 127 | 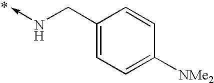 | 529 |
| 128 | 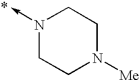 | 479 |
| 129 | 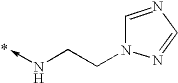 | 491 |
| 130 | 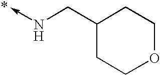 | 494 |
| 131 | 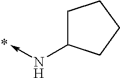 | 464 |
| 132 | 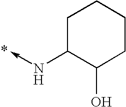 | 494 |
| 133 | 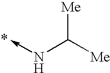 | 436 |
| 134 | 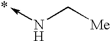 | 424 |
| 135 | 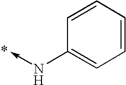 | 470 |
| 136 | 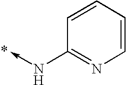 | 471 |
| 137 | 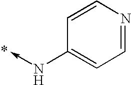 | 471 |
| 138 | 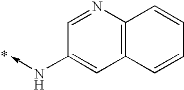 | 521 |
| 139 | 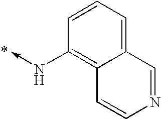 | 521 |
| 140 | 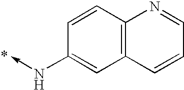 | 521 |
| 141 | 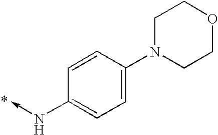 | 555 |

### Working Example 142

### N-(2-Hydroxyethyl)-3-[1-methyl-2-[[3-(trifluoromethyl)phenyl]amino]-1H-benzimidazol-4-yl]benzamide

3-[1-Methyl-2-[[3-(trifluoromethyl)phenyl]amino]-1H-benzimidazol-4-yl]benzoic acid obtained in Reference Example 233 and 2-aminoethanol were used in the same manner as in Working Example 3 to obtain the titled compound. Yield: 50%; melting point: 235-236° C. (ethanol).

¹H-NMR (DMSO-d₆) δ: 3.38 (2H, t, J=5.8 Hz), 3.54 (2H, q, J=6.0 Hz), 3.79 (3H, s), 4.73 (1H,t, J=5.6 Hz), 7.21 (1H, t, J=7.8 Hz), 7.28 (1H, d, J=7.7 Hz), 7.37 (1H, d, J=7.7 Hz), 7.43-7.61 (3H, m), 7.81 (1H, d, J=7.7 Hz), 8.10 (1H, d, J=8.2 Hz), 8.43-8.58 (3H, m), 8.66 (1H, s), 9.44 (1H, s)

### Working Example 143

### N-(2-Methoxyethyl)-3-[1-methyl-2-[3-(trifluoromethyl)phenoxy]-1H-benzimidazol-4-yl]benzamide

3-[1-Methyl-2-[3-(trifluoromethyl)phenoxy]-1H-benzimidazol-4-yl]benzoic acid obtained in Reference Example 234 and 2-methoxyethane were used in the same manner as in Working Example 3 to obtain the titled compound. Yield: 34%; melting point: 146-147° C. (ethyl acetate-hexane).

¹H-NMR (CDCl₃) δ: 3.35 (3H, s), 3.51-3.57 (2H, m), 3.65 (2H, q, J=5.1 Hz), 3.79 (3H, s), 6.53 (1H, br. s.), 7.28 (1H, d, J=1.1 Hz), 7.34 (1H, t, J=7.7 Hz), 7.44-7.51 (3H, m), 7.56 (1H, t, J=8.0 Hz), 7.71-7.79 (2H, m), 7.86 (1H, s), 8.16 (1H, ddd, J=8.0, 1.5, 1.2 Hz), 8.32 (1H, t, J=1.6 Hz)

### Working Example 144

### N-(2-Amino-2-oxoethyl)-3-[1-methyl-2-[3-(trifluoromethyl)phenoxy]-1H-benzimidazol-4-yl]benzamide

3-[1-Methyl-2-[3-(trifluoromethyl)phenoxy]-1H-benzimidazol-4-yl]benzoic acid obtained in Reference Example 234 and glycinamide hydrochloride were used in the same manner as in Working Example 12 to obtain the titled compound. Yield: 30%; melting point: 185-186° C. (ethyl acetate).

¹H-NMR (DMSO-d₆) δ: 3.74-3.90 (5H, m), 7.03 (1H, br s.), 7.29-7.41 (2H, m), 7.45 (1H, t, J=7.8 Hz), 7.51 (2H, dd, J=7.7, 3.0 Hz), 7.59-7.77 (3H, m), 7.83 (2H, dd, J=18.0, 7.8 Hz), 8.05 (1H, s), 8.20 (1H, d, J=7.7 Hz), 8.35 (1H, s), 8.70 (1H, t, J=5.9 Hz)

### Working Example 145

### N-(2-Hydroxyethyl)-3-(1-methyl-2-[[3-(trifluoromethyl)phenyl]sulfanyl]-1H-benzimidazol-4-yl)benzamide

3-(1-Methyl-2-[[3-(trifluoromethyl)phenyl]sulfanyl]-1H-benzimidazol-4-yl)benzoic acid obtained in Reference Example 235 and 2-aminoethanol were used in the same manner as in Working Example 3 to obtain the titled compound. Yield: 72%; melting point: 138-139° C. (ethyl acetate-hexane).

¹H-NMR (CDCl₃) δ: 3.29 (1H, t, J=5.4 Hz), 3.55-3.64 (2H, m), 3.75-3.86 (5H, m), 7.03 (1H, t, J=5.6 Hz), 7.29-7.36 (1H, m), 7.37-7.56 (6H, m), 7.70-7.79 (2H, m), 8.12 (1H, dt, J=7.8, 1.5 Hz), 8.32 (1H, t, J=1.6 Hz).

### Working Example 146

### 3-[2-(3-Chlorobenzyl)-1-benzofuran-7-yl]-N-(2-cyanoethyl)benzamide

3-[2-(3-Chlorobenzyl)-1-benzofuran-7-yl]benzoic acid obtained in Reference Example 236 and 3-aminopropanenitrile were used in the same manner as in Working Example 3 to obtain the titled compound. Yield: 97%.

¹H-NMR (CDCl₃) δ: 2.77 (2H, t, J=6.2 Hz), 3.74 (2H, q, J=12.4, 6.4 Hz), 4.12 (2H, s), 6.46 (1H, s), 6.57 (1H, t, J=5.6 Hz), 7.19-7.33 (5H, m), 6.57 (1H, dd, J=7.6, 1.2 Hz), 7.50 (1H, dd, J=7.6, 1.2 Hz), 7.58 (1H, t, J=7.6 Hz), 7.79 (1H, dt, J=1.4, 8.0 Hz), 7.99 (1H, dt, J=1.4, 8.0 Hz), 8.21 (1H, t, J=1.6 Hz).

### Working Example 147

### 3-[2-(3-Chlorobenzyl)-1-benzofuran-7-yl]-N-(2-hydroxyethyl)benzamide

3-[2-(3-Chlorobenzyl)-1-benzofuran-7-yl]benzoic acid obtained in Reference Example 236 and 2-aminoethanol were used in the same manner as in Working Example 3 to obtain the titled compound. Yield: 98%.

¹H-NMR (CDCl₃) δ: 2.54 (1H, s), 3.66 (2H, q, J=9.8, 5.4 Hz), 3.86 (2H, d, J=3.6 Hz), 4.12 (2H, s), 6.46 (1H, s), 6.61 (1H, s), 7.20-7.33 (5H, m), 7.42 (1H, dd, J=7.6, 1.2 Hz), 7.49 (1H, dd, J=7.6, 1.2 Hz), 7.56 (1H, t, J=7.8 Hz), 7.79 (1H, dt, J=1.4, 8.0 Hz), 7.97 (1H, dt, J=1.4, 8.0 Hz), 8.21 (1H, t, J=1.6 Hz).

### Working Example 148

### N-(2-Amino-2-oxoethyl)-3-[3-methyl-2-[3-(trifluoromethyl)benzyl]-1-benzofuran-7-yl]benzamide

3-[3-Methyl-2-[3-(trifluoromethyl)benzyl]-1-benzofuran-7-yl]benzoic acid obtained in Reference Example 32 and glycinamide hydrochloride were used in the same manner as in Working Example 12 to obtain the titled compound. Yield: 62%; melting point: 194-195° C. (ethyl acetate).

¹H-NMR (DMSO-d₆) δ: 2.28 (3H, s), 3.85 (2H, d, J=5.8 Hz), 4.30 (2H, s), 7.05 (1H, br s.), 7.30-7.42 (2H, m), 7.50-7.67 (6H, m), 7.71 (1H, s), 7.88 (1H, d, J=8.0 Hz), 7.97 (1H, d, J=8.2 Hz), 8.31 (1H, s), 8.78 (1H, t, J=5.6 Hz)

### Working Example 149

### N-(2-Cyanoethyl)-3-[2-[3-(trifluoromethyl)benzyl]-1-benzothiophen-7-yl]benzamide

1 N sodium hydroxide aqueous solution (2.72 mL, 2.72 mmol) was added to an ethanol (10 mL)-THF (2 mL) solution of ethyl 3-[2-[3-(trifluoromethyl)benzyl]-1-benzothiophen-7-yl]benzoate (0.40 g, 0.91 mmol) obtained in Reference Example 200, and the mixture was stirred for 30 min at 60° C. The reaction solution was neutralized with the addition of 1 N hydrochloric acid (2.72 mL), and the solvent was distilled off at reduced pressure. A mixture of the residue, 3-aminopropanenitrile (0.078 mL, 1.09 mmol), WSC (0.23 g, 1.36 mmol), HOBt (0.18 g, 1.36 mmol), and DMF (7 mL) was stirred for 3 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate =1:1), and the resulting crystals were recrystallized from hexane-ethyl acetate to give 0.35 g of the titled compound (yield 83%). Melting point: 95-98° C.

¹H NMR (CDCl₃) δ: 2.76 (2H, t, J=6.3 Hz), 3.73 (2H, q, J=6.3 Hz), 4.27 (2H, s), 6.55-6.75 (1H, m), 7.10 (1H, s), 7.31 (1H, d, J=7.5 Hz), 7.40-7.60 (6H, m), 7.69 (1H, d, J=7.8 Hz), 7.79 (1H, d, J=8.1 Hz), 7.84 (1H, d, J=7.8 Hz), 8.05 (1H, t, J=7.8 Hz).

### Working Example 150

### N-(2-Amino-2-oxoethyl)-3-[2-[3-(trifluoromethyl)benzyl]-1-benzothiophen-7-yl]benzamide

1 N sodium hydroxide aqueous solution (2.72 mL, 2.72 mmol) was added to an ethanol (10 mL)-THF (2 mL) solution of ethyl 3-[2-[3-(trifluoromethyl)benzyl]-1-benzothiophen-7-yl]benzoate (0.40 g, 0.91 mmol) obtained in Reference Example 200, and the mixture was stirred for 30 min at 60° C. The reaction solution was neutralized with the addition of 1 N hydrochloric acid (2.72 mL), and the solvent was distilled off at reduced pressure. A mixture of the residue, glycinamide hydrochloride (0.12 g, 1.09 mmol), triethylamine (0.15 mL, 1.09 mmol) WSC (0.23 g, 1.36 mmol), HOBt (0.18 g, 1.36 mmol), and DMF (7 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate), and the resulting crystals were recrystallized from hexane-ethyl acetate to give 0.40 g of the titled compound (yield 94%).

Melting point: 147-148° C.

¹H NMR (CDCl₃) δ: 4.18 (2H, d, J=4.8 Hz), 4.27 (1H, s), 5.45 (1H, br s), 6.00 (1H, br s), 6.90-7.00 (1H, m), 7.10 (1H, s), 7.31 (1H, d, J=7.2 Hz), 7.40-7.60 (6H, m), 7.68 (1H, d, J=8.1 Hz), 7.80-7.90 (2H, m), 8.10 (1H, s).

### Working Example 151

### N-(2-Methoxyethyl)-3-[2-[3-(trifluoromethyl)benzyl]-1-benzothiophen-7-yl]benzamide

1 N sodium hydroxide aqueous solution (1.70 mL, 1.70 mmol) was added to an ethanol (10 mL)-THF (2 mL) solution of ethyl 3-[2-[3-(trifluoromethyl)benzyl]-1-benzothiophen-7-yl]benzoate (0.25 g, 0.57 mmol) obtained in Reference Example 200, and the mixture was stirred for 30 min at 60° C. The reaction solution was neutralized with the addition of 1 N hydrochloric acid (1.70 mL), and the solvent was distilled off at reduced pressure. A mixture of the residue, 2-methoxyethanamine (0.059 mL, 0.68 mmol), WSC (0.15 g, 0.85 mmol), HOBt (0.12 g, 0.85 mmol), and DMF (4 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=2:1), and the resulting crystals were recrystallized from hexane-ethyl acetate to give 0.24 g of the titled compound (yield 90%).

Melting point: 113-114° C.

¹H NMR (CDCl₃) δ: 3.37 (3H, s), 3.56 (2H, d, J=5.0 Hz), 3.67 (2H, q, J=5.0 Hz), 4.27 (2H, s), 6.53 (1H, br s), 7.10 (1H, s), 7.32 (1H, d, J=7.5 Hz), 7.40-7.60 (6H, m), 7.69 (1H, d, J=7.8 Hz), 7.75-7.85 (2H, m), 8.04 (1H, s).

### Working Example 152

### N-(2-Hydroxyethyl)-3-[2-[3-(trifluoromethyl)benzyl]-1-benzothiophen-7-yl]benzamide

1 N sodium hydroxide aqueous solution (1.70 mL, 1.70 mmol) was added to an ethanol (5 mL)-THF (2 mL) solution of ethyl 3-[2-[3-(trifluoromethyl)benzyl]-1-benzothiophen-7-yl]benzoate (0.25 g, 0.57 mmol) obtained in Reference Example 200, and the mixture was stirred for 30 min at 60° C. The reaction solution was neutralized with the addition of 1 N hydrochloric acid (1.70 mL), and the solvent was distilled off at reduced pressure. A mixture of the residue, 2-aminoethanol (0.041 mL, 0.68 mmol), WSC (0.15 g, 0.85 mmol), HOBt (0.12 g, 0.85 mmol), and DMF (5 mL) was stirred for 15 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=1:1) to give 0.25 g of the titled compound (yield 97%) in the form of an oily substance.

¹H NMR (CDCl₃) δ: 2.24 (1H, t, J=4.8 Hz), 3.65 (1H, q, J=5.1 Hz), 3.85 (1H, q, J=5.1 Hz), 4.27 (2H, s), 6.62 (1H, br s), 7.11 (1H, s), 7.31 (1H, d, J=7.5 Hz), 7.40-7.60 (6H, m), 7.69 (1H, d, J=7.5 Hz), 7.78-7.85 (2H, m), 8.05 (1H, s).

### Working Example 153

### N-(2-Methoxyethyl)-3-[2-[3-(methylsulfonyl)benzyl]-1-benzothiophen-7-yl]benzamide

1 N sodium hydroxide aqueous solution (2.0 mL, 2.0 mmol) was added to an ethanol (5 mL) solution of ethyl 3-[2-[3-(methylsulfonyl)benzyl]-1-benzothiophen-7-yl]benzoate (0.3 g, 0.66 mmol) obtained in Reference Example 201, and the mixture was stirred for 1 hour at 50° C. The reaction solution was neutralized with the addition of 1 N hydrochloric acid (2.0 mL), and the solvent was distilled off at reduced pressure. A mixture of the residue, 2-methoxyethanamine (0.069 mL, 0.80 mmol), WSC (0.17 g, 1.0 mmol), HOBt (0.135 g, 1.0 mmol), and DMF (5 mL) was stirred for 3 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate:methanol=10:1) and recrystallized from hexane-ethyl acetate to give 0.27 g of the titled compound (yield 85%) in the form of crystals.

Melting point: 131-132° C.

¹H NMR (CDCl₃) δ: 3.04 (3H, s), 3.37 (3H, s), 3.56 (2H, t, J=4.8 Hz), 3.67 (2H, q, J=4.8 Hz), 4.31 (2H, s), 6.55 (1H, s), 7.14 (1H, s), 7.33 (1H, d, J=7.2 Hz), 7.44 (1H, t, J=7.5 Hz), 7.50-7.60 (3H, m), 7.70 (1H, d, J=7.5 Hz), 7.75-7.85 (3H, m), 7.86 (1H, s), 8.05 (1H, s).

### Working Example 154

### N-(2-Amino-2-oxoethyl)-3-[2-[3-(methylsulfonyl)benzyl]-1-benzothiophen-7-yl]benzamide

1 N sodium hydroxide aqueous solution (2.33 mL, 2.33 mmol) was added to an ethanol (5 mL) solution of ethyl 3-[2-[3-(methylsulfonyl)benzyl]-1-benzothiophen-7-yl]benzoate (0.35 g, 0.78 mmol) obtained in Reference Example 201, and the mixture was stirred for 1 hour at 50° C. The reaction solution was neutralized with the addition of 1 N hydrochloric acid (2.33 mL), and the solvent was distilled off at reduced pressure. A mixture of the residue, glycinamide hydrochloride (103 mg, 0.93 mmol), WSC (0.20 g, 1.17 mmol), HOBt (0.16 g, 1.17 mmol), triethylamine (0.13 mL, 0.93 mmol), and DMF (5 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate:methanol=4:1), and the resulting crystals were recrystallized from hexane-ethyl acetate to give 0.34 g of the titled compound (yield 91%).

Melting point: 189-193° C.

¹H NMR (d₆-DMSO) δ: 3.20 (3H, s), 3.81 (2H, d, J=5.7 Hz), 4.40 (2H, s), 7.04 (1H, S), 7.35-7.45 (3H, m), 7.49 (1H, t, J=7.5 Hz), 7.55-7.65 (2H, m), 7.68 (1H, d, J=7.5 Hz), 7.70-7.85 (3H, m), 7.89 (1H, s), 7.93 (1H, d, J=8.1 Hz), 8.15 (1H, s), 8.75-8.85 (1H, M).

### Working Example 155

### 3-[2-[3-Fluoro-5-(trifluoromethyl)benzyl]-1-benzothiophen-7-yl]-N-(2-methoxyethyl)benzamide

1 N sodium hydroxide aqueous solution (1.96 mL, 1.96 mmol) was added to an ethanol (10 mL) solution of ethyl 3-[2-[3-fluoro-5-(trifluoromethyl)benzyl]-1-benzothiophen-7-yl]benzoate (0.3 g, 0.64 mmol) obtained in Reference Example 202, and the mixture was stirred for 30 min at 50° C. The reaction solution was neutralized with the addition of 1 N hydrochloric acid (1.96 mL), and the solvent was distilled off at reduced pressure. A mixture of the residue, 2-methoxyethanamine (0.064 mL, 0.74 mmol), WSC (159 mg, 0.92 mmol), HOBt (125 mg, 0.92 mmol), and DMF (5 mL) was stirred for 15 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=2:1-1:2) and recrystallized from hexane-ethyl acetate to give 0.22 g of the titled compound (yield 69%).

Melting point: 106-107° C.

¹H NMR (CDCl₃) δ: 3.37 (3H, s), 3.56 (2H, t, J=5.0 Hz), 3.67 (2H, q, J=5.0 Hz), 4.26 (2H, s), 6.54 (1H, m), 7.14 (1H, s), 7.18 (2H, t, J=9.6 Hz), 7.30-7.40 (2H, m), 7.47 (1H, t, J=8.1 Hz), 7.54 (1H, t, J=7.8 Hz), 7.71 (1H, d, J=8.1 Hz), 7.75-7.85 (2H, m), 8.05 (1H, s).

### Working Example 156

### N-(2-Amino-2-oxoethyl)-3-[2-[3-fluoro-5-(trifluoromethyl)benzyl]-1-benzothiophen-7-yl]benzamide

1 N sodium hydroxide aqueous solution (1.96 mL, 1.96 mmol) was added to an ethanol (10 mL) solution of ethyl 3-[2-[3-fluoro-5-(trifluoromethyl)benzyl]-1-benzothiophen-7-yl]benzoate (0.3 g, 0.64 mmol) obtained in Reference Example 202, and the mixture was stirred for 30 min at 50° C. The reaction solution was neutralized with the addition of 1 N hydrochloric acid (1.96 mL), and the solvent was distilled off at reduced pressure. A mixture of the residue, glycinamide hydrochloride (87 mg, 0.74 mmol), WSC (159 mg, 0.92 mmol), HOBt (125 mg, 0.92 mmol), triethylamine (0.11 mL, 0.74 mmol), and DMF (5 mL) was stirred for 2 hours. The reaction solution was diluted with water and extracted with ethyl acetate. The extract was washed with water and dried over anhydrous magnesium sulfate, and was then passed through a small amount of silica gel to allow the solvent to be distilled off at reduced pressure. The residue was crystallized from hexane-ethyl acetate to give 0.24 g of the titled compound (yield 75%).

Melting point: 169-170° C.

¹H NMR (CDCl₃) δ: 4.19 (2H, d, J=4.8 Hz), 4.26 (2H, s), 5.48 (1H, m), 6.09 (1H, m), 7.01 (1H, m), 7.13 (1H, m), 7.18 (2H, t, J=9.0 Hz), 7.30-7.40 (2H, m), 7.43 (1H, t, J=7.5 Hz), 7.55 (1H, t, J=7.5 Hz), 7.70 (1H, d, J=7.8 Hz), 7.87 (2H, d, J=7.8 Hz), 8.10 (1H, s).

### Working Example 157

### 3-[2-[3-Fluoro-5-(trifluoromethyl)benzyl]-1-benzothiophen-7-yl]benzamide

1 N sodium hydroxide aqueous solution (1.96 mL, 1.96 mmol) was added to an ethanol (5 mL) solution of ethyl 3-[2-[3-fluoro-5-(trifluoromethyl)benzyl]-1-benzothiophen-7-yl]benzoate (0.3 g, 0.65 mmol) obtained in Reference Example 202, and the mixture was stirred for 30 min at 50° C. The reaction solution was neutralized with the addition of 1 N hydrochloric acid (1.96 mL), and the solvent was distilled off at reduced pressure. A mixture of the residue, WSC (0.16 g, 0.92 mmol), HOBt (0.13 g, 0.92 mmol), and DMF (5 mL) was stirred for 1 hour, the reaction solution was then added to 28% aqueous ammonia (10 mL), and the mixture was stirred for 30 min. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=4:1-1:4), and the resulting crystals were recrystallized from hexane-ethyl acetate to give 0.26 g of the titled compound (yield 93%).

Melting point: 147-148° C.

¹H NMR (CDCl₃) δ: 4.26 (2H, s), 5.60 (1H, br s), 6.10 (1H, br s), 7.14 (1H, s), 7.18-7.25 (2H, m), 7.30-7.40 (2H, m), 7.44 (1H, t, J=7.2 Hz), 7.56 (1H, t, J=7.8 Hz), 7.71 (1H, d, J=8.1 Hz), 7.84 (2H, d, J=7.2 Hz), 8.09 (1H, s).

### Working Example 158

### N-(2-Amino-2-oxoethyl)-3-[4-fluoro-2-[3-(methylsulfonyl)benzyl]-1-benzothiophen-7-yl]benzamide

1 N sodium hydroxide aqueous solution (2.24 mL, 2.24 mmol) was added to an ethanol (5 mL) solution of ethyl 3-[4-fluoro-2-[3-(methylsulfonyl)benzyl]-1-benzothiophen-7-yl]benzoate (0.35 g, 0.75 mmol) obtained in Reference Example 203, and the mixture was stirred for 30 min at 50° C. The reaction solution was neutralized with the addition of 1 N hydrochloric acid (2.24 mL), and the solvent was distilled off at reduced pressure. A mixture of the residue, glycinamide hydrochloride (99 mg, 0.90 mmol), WSC (0.19 g, 1.12 mmol), HOBt (0.15 g, 1.12 mmol), triethylamine (0.13 mL, 0.90 mmol), and DMF (5 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate:methanol=4:1), and the resulting crystals were recrystallized from ethanol-diethyl ether to give 0.27 g of the titled compound (yield 69%).

Melting point: 107-108° C.

¹H NMR (CDCl₃) δ: 3.05 (3H, s), 4.18 (2H, d, J=5.1 Hz), 4.31 (2H, s), 5.50-5.60 (1H, br s), 6.00-6.15 (1H, br s), 7.09 (1H, t, J=8.9 Hz), 7.00-7.10 (1H, m), 7.20-7.30 (2H, m), 7.45-7.65 (3H, m), 7.76 (1H, d, J=7.8 Hz), 7.80-7.90 (3H, m), 8.06 (1H, s).

### Working Example 159

### 3-[4-Fluoro-2-[3-(methylsulfonyl)benzyl]-1-benzothiophen-7-yl]-N-(2-methoxyethyl)benzamide

1 N sodium hydroxide aqueous solution (2.56 mL, 2.56 mmol) was added to an ethanol (5 mL) solution of ethyl 3-[4-fluoro-2-[3-(methylsulfonyl)benzyl]-1-benzothiophen-7-yl]benzoate (0.4 g, 0.85 mmol) obtained in Reference Example 203, and the mixture was stirred for 30 min at 50° C. The reaction solution was neutralized with the addition of 1 N hydrochloric acid (2.56 mL), and the solvent was distilled off at reduced pressure. A mixture of the residue, 2-methoxyethanamine (0.089 mL, 1.02 mmol), WSC (0.22 g, 1.28 mmol), HOBt (0.17 g, 1.28 mmol), and DMF (5 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=1:1, then ethyl acetate), and the resulting crystals were recrystallized from hexane-ethyl acetate to give 0.30 g of the titled compound (yield 71%).

Melting point: 158-159° C.

¹H NMR (CDCl₃) δ: 3.04 (3H, s), 3.38 (3H, s), 3.56 (2H, t, J=4.8 Hz), 3.67 (1H, q, J=4.8 Hz), 4.31 (2H, s), 6.54 (1H, m), 7.10 (1H, t, J=8.9 Hz), 7.20-7.30 (2H, m), 7.45-7.60 (3H, m), 7.70-7.90 (4H, m), 8.02 (1H, s).

### Working Example 160

### N-(2-Amino-2-oxoethyl)-3-[2-[3-fluoro-5-(methylsulfonyl)benzyl]-1-benzothiophen-7-yl]benzamide

1 N sodium hydroxide aqueous solution (2.24 mL, 2.24 mmol) was added to an ethanol (5 mL) solution of ethyl 3-[2-[3-fluoro-5-(methylsulfonyl)benzyl]-1-benzothiophen-7-yl]benzoate (0.35 g, 0.75 mmol) obtained in Reference Example 257, and the mixture was stirred for 30 min at 50° C. The reaction solution was neutralized with the addition of 1 N hydrochloric acid (2.24 mL), and the solvent was distilled off at reduced pressure. A mixture of the residue, glycinamide hydrochloride (99 mg, 0.90 mmol), WSC (0.19 g, 1.12 mmol), HOBt (0.15 g, 1.12 mmol), triethylamine (0.13 mL, 0.90 mmol), and DMF (5 mL) was stirred for 5.5 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate:methanol=10:1), and the resulting crystals were recrystallized from ethanol-ethyl acetate to give 0.20 g of the titled compound (yield 54%).

Melting point: 160-161° C.

¹H NMR (CDCl₃) δ: 3.05 (3H, s), 4.18 (2H, q, J=5.1 Hz), 4.30 (1H, s), 5.49 (1H, br s), 6.00 (1H, br s), 6.98 (1H, br s), 7.18 (1H, s), 7.20-7.30 (1H, m), 7.34 (1H, d, J=7.2 Hz), 7.45 (1H, t, J=7.6 Hz), 7.52 (1H, d, J=9.0 Hz), 7.56 (1H, t, J=7.6 Hz), 7.67 (1H, s), 7.71 (1H, d, J=7.5 Hz), 7.80-7.90 (2H, m), 8.12 (1H, s).

### Working Example 161

### 3-[2-[3-Fluoro-5-(methylsulfonyl)benzyl]-1-benzothiophen-7-yl]benzamide

1 N sodium hydroxide aqueous solution (2.24 mL, 2.24 mmol) was added to an ethanol (5 mL) solution of ethyl 3-[2-[3-fluoro-5-(methylsulfonyl)benzyl]-1-benzothiophen-7-yl]benzoate (0.35 g, 0.75 mmol) obtained in Reference Example 257, and the mixture was stirred for 30 min at 50° C. The reaction solution was neutralized with the addition of 1 N hydrochloric acid (2.24 mL), and the solvent was distilled off at reduced pressure. A mixture of the residue, WSC (0.19 g, 1.12 mmol), HOBt (0.15 g, 1.12 mmol), and DMF (5 mL) was stirred for 2 hours, the reaction solution was added to 28% aqueous ammonia (15 mL), and the mixture was stirred for 30 min. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=1:2, then ethyl acetate), and the resulting crystals were recrystallized from hexane-ethyl acetate to give 0.18 g of the titled compound (yield 55%).

Melting point: 170-171° C.

¹H NMR (CDCl₃) δ: 3.05 (3H, s), 4.30 (2H, s), 5.62 (1H, br s), 6.12 (1H, br s), 7.17 (1H, s), 7.20-7.35 (1H, m), 7.34 (1H, d, J=7.5 Hz), 7.45 (1H, t, J=7.6 Hz), 7.50-7.67 (1H, m), 7.58 (1H, d, J=7.6 Hz), 7.70 (1H, s), 7.71 (1H, d, J=7.5 Hz), 7.82 (1H, s), 7.85 (1H, s), 8.10 (1H, s).

### Working Example 162

### N-(2-Cyanoethyl)-3-[2-[3-(trifluoromethyl)benzyl]-1-benzothiophen-4-yl]benzamide

2 N sodium hydroxide aqueous solution (0.23 mL, 0.46 mmol) was added to a methanol (1 mL)-THF (2 mL) solution of ethyl 3-[2-[3-(trifluoromethyl)benzyl]-1-benzothiophen-4-yl]benzoate (101 mg, 0.23 mmol) obtained in Reference Example 197, the mixture was stirred for 2 hours and 30 min at room temperature, 2 N sodium hydroxide aqueous solution (0.23 mL, 0.46 mmol) was then further added, and the mixture was stirred for 2 hours and 30 min at room temperature. The reaction solution was neutralized with the addition of 1 N hydrochloric acid, then diluted with water, and extracted with ethyl acetate. The extract was washed with saturated brine and dried over anhydrous sodium sulfate, and the solvent was then distilled off at reduced pressure to give a 3-[2-[3-(trifluoromethyl)benzyl]-1-benzothiophen-4-yl]benzoic acid crude product. A mixture of the resulting 3-[2-[3-(trifluoromethyl)benzyl]-1-benzothiophen-4-yl]benzoic acid crude product, 3-aminopropanenitrile (0.022 mL, 0.30 mmol), WSC (57 mg, 0.30 mmol), HOBt (40 mg, 0.30 mmol), and DMF (3 mL) was stirred for 14 hours. The reaction solution was diluted with saturated sodium bicarbonate aqueous solution and extracted with ethyl acetate. The extract was washed with saturated brine and dried over anhydrous magnesium sulfate, and the solvent was then distilled off at reduced pressure. The residue was purified by silica gel column chromatography (hexane:ethyl acetate=90:10→50:50), and the resulting crystals were recrystallized from hexane-ethyl acetate to give 0.60 g of the titled compound (yield 56%). Melting point: 137-138° C.

¹H-NMR (CDCl₃) δ: 2.74-2.81 (2H, m), 3.70-3.79 (2H, m), 4.27 (2H, s), 6.60 (1H, br s), 7.13 (1H, d, J=0.8 Hz), 7.27-7.61 (7H, m), 7.69-7.74 (1H, m), 7.74-7.78 (1H, m), 7.78-7.82 (1H, m), 7.93-7.96 (1H, m).

### Working Example 163

### N-(2-Methoxyethyl)-3-[2-[3-(trifluoromethyl)benzyl]-1-benzothiophen-4-yl]benzamide

A 3-[2-[3-(trifluoromethyl)benzyl]-1-benzothiophen-4-yl]benzoic acid crude product was synthesized from ethyl 3-[2-[3-(trifluoromethyl)benzyl]-1-benzothiophen-4-yl]benzoate obtained in Reference Example 197, and the resulting 3-[2-[3-(trifluoromethyl)benzyl]-1-benzothiophen-4-yl]benzoic acid crude product and 2-methoxyethanamine were used in the same manner as in Working Example 162 to obtain the titled compound. 83% yield, oily substance.

¹H-NMR (CDCl₃) δ: 3.38 (3H, s), 3.55-3.61 (2H, m), 3.64-3.73 (2H, m), 4.27 (2H, s), 6.54 (1H, br s), 7.15 (1H, d, J=0.8 Hz), 7.29-7.58 (7H, m), 7.65-7.70 (1H, m), 7.73-7.78 (1H, m), 7.78-7.83 (1H, m), 7.93-7.96 (1H, m).

### Working Example 164

### N-(2-Hydroxyethyl)-3-[2-[3-(trifluoromethyl)benzyl]-1-benzothiophen-4-yl]benzamide

2 N sodium hydroxide aqueous solution (0.89 mL, 1.78 mmol) was added to a methanol (3 mL)-THF (6 mL) solution of ethyl 3-[2-[3-(trifluoromethyl)benzyl]-1-benzothiophen-4-yl]benzoate (392 mg, 0.89 mmol) obtained in Reference Example 197, and the mixture was stirred for 3 hours at room temperature. The reaction solution was neutralized with the addition of 1 N hydrochloric acid, then diluted with water, and extracted with ethyl acetate. The extract was washed with saturated brine and dried over anhydrous sodium sulfate, and the solvent was then distilled off at reduced pressure to give a 3-[2-[3-(trifluoromethyl)benzyl]-1-benzothiophen-4-yl]benzoic acid crude product (362 mg). A mixture of the resulting 3-[2-[3-(trifluoromethyl)benzyl]-1-benzothiophen-4-yl]benzoic acid crude product (112 mg), 2-aminoethanol (0.015 mL, 0.353 mmol), DMTMM (104 mg, 0.353 mmol), and methanol (3 mL) was stirred for 14 hours. The reaction solution was diluted with saturated sodium bicarbonate aqueous solution and extracted with ethyl acetate. The extract was washed with saturated brine and dried over anhydrous magnesium sulfate, and the solvent was then distilled off at reduced pressure. The residue was purified by silica gel column chromatography (hexane:ethyl acetate=30:70→0:100) to give 105 mg of the titled compound (yield 85%). Oily substance.

¹H-NMR (CDCl₃) δ: 2.41 (1H, t, J=4.9 Hz), 3.61-3.72 (2H, m), 3.81-3.91 (2H, m), 4.27 (2H, s), 6.62 (1H, br s), 7.13 (1H, d, J=0.8 Hz), 7.28-7.59 (7H, m), 7.66-7.71 (1H, m), 7.73-7.78 (1H, m), 7.79-7.84 (1H, m), 7.93-7.96 (1H, m).

### Working Example 165

### N-(2-Amino-2-oxoethyl)-3-[2-[3-(trifluoromethyl)benzyl]-1-benzothiophen-4-yl]benzamide

A 3-[2-[3-(trifluoromethyl)benzyl]-1-benzothiophen-4-yl]benzoic acid crude product was synthesized from ethyl 3-[2-[3-(trifluoromethyl)benzyl]-1-benzothiophen-4-yl]benzoate obtained in Reference Example 197, and the resulting 3-[2-[3-(trifluoromethyl)benzyl]-1-benzothiophen-4-yl]benzoic acid crude product and glycinamide hydrochloride were used in the same manner as in Working Example 12 to obtain the titled compound. Yield: 92%. Melting point: 125-126° C. (hexane-ethyl acetate).

¹H-NMR (CDCl₃) δ: 4.19 (2H, d, J=4.9 Hz), 4.27 (2H, s), 5.46 (1H, br s), 5.96 (1H, br s), 6.96 (1H, br s), 7.13 (1H, d, J=0.8 Hz), 7.27-7.61 (7H, m), 7.67-7.78 (2H, m), 7.81-7.87 (1H, m), 7.99 (1H, t, J=1.6 Hz).

### Working Example 166

### 3-[2-(3-Chloro-4-fluorobenzyl)-1-benzothiophen-4-yl]-N-(2-cyanoethyl)benzamide

A 3-[2-(3-chloro-4-fluorobenzyl)-1-benzothiophen-4-yl]benzoic acid crude product was synthesized from ethyl 3-[2-(3-chloro-4-fluorobenzyl)-1-benzothiophen-4-yl]benzoate obtained in Reference Example 198, and the resulting 3-[2-(3-chloro-4-fluorobenzyl)-1-benzothiophen-4-yl]benzoic acid crude product and 3-aminopropanenitrile were used in the same manner as in Working Example 162 to obtain the titled compound. Yield: 93%. Melting point: 133-135° C. (hexane-ethyl acetate).

¹H-NMR (CDCl₃) δ: 2.78 (2H, t, J=6.3 Hz), 3.70-3.79 (2H, m), 4.16 (2H, s), 6.58 (1H, br s), 7.04-7.13 (3H, m), 7.21-7.40 (3H, m), 7.54-7.61 (1H, m), 7.69-7.83 (3H, m), 7.93-7.96 (1H, m).

### Working Example 167

### N-(2-Amino-2-oxoethyl)-3-[2-(3-chloro-4-fluorobenzyl)-1-benzothiophen-4-yl]benzamide

A 3-[2-(3-chloro-4-fluorobenzyl)-1-benzothiophen-4-yl]benzoic acid crude product was synthesized from ethyl 3-[2-(3-chloro-4-fluorobenzyl)-1-benzothiophen-4-yl]benzoate obtained in Reference Example 198, and the resulting 3-[2-(3-chloro-4-fluorobenzyl)-1-benzothiophen-4-yl]benzoic acid crude product and glycinamide hydrochloride were used in the same manner as in Working Example 12 to 10 obtain the titled compound. Yield: 92%. Melting point: 158-159° C. (hexane-ethyl acetate).

¹H-NMR (CDCl₃) δ: 4.15 (2H, s), 4.20 (2H, d, J=5.2 Hz), 5.52 (1H, br s), 6.14 (1H, br s), 7.00-7.18 (4H, m), 7.25-7.37 (3H, m), 7.54 (1H, d, J=7.7 Hz), 7.66-7.77 (2H, m), 7.81-7.86 (1H, m), 7.97-8.01 (1H, m).

### Working Example 168

### 3-[2-(3-Chloro-4-fluorobenzyl)-1-benzothiophen-4-yl]-N-(2-methoxyethyl)benzamide

A 3-[2-(3-chloro-4-fluorobenzyl)-1-benzothiophen-4-yl]benzoic acid crude product was synthesized from ethyl 3-[2-(3-chloro-4-fluorobenzyl)-1-benzothiophen-4-yl]benzoate obtained in Reference Example 198, and the resulting 3-[2-(3-chloro-4-fluorobenzyl)-1-benzothiophen-4-yl]benzoic acid crude product and 2-methoxyethanamine were used in the same manner as in Working Example 162 to obtain the titled compound. Yield: quantitative. Oily substance.

¹H-NMR (CDCl₃) δ: 3.38 (3H, s), 3.54-3.62 (2H, m), 3.64-3.73 (2H, m), 4.16 (2H, s), 6.54 (1H, br s), 7.01-7.15 (3H, m), 7.26-7.39 (3H, m), 7.54 (1H, t, J=7.7 Hz), 7.64-7.69 (1H, m), 7.73-7.78 (1H, m), 7.78-7.84 (1H, m), 7.95 (1H, t, J=1.6 Hz).

### Working Example 169

### 3-[2-(3-Chloro-5-fluorobenzyl)-1-benzothiophen-4-yl]-N-(2-cyanoethyl)benzamide

A 3-[2-(3-chloro-5-fluorobenzyl)-1-benzothiophen-4-yl]benzoic acid crude product was synthesized from ethyl 3-[2-(3-chloro-5-fluorobenzyl)-1-benzothiophen-4-yl]benzoate obtained in Reference Example 199, and the resulting 3-[2-(3-chloro-5-fluorobenzyl)-1-benzothiophen-4-yl]benzoic acid crude product and 3-aminopropanenitrile were used in the same manner as in Working Example 162 to obtain the titled compound. Yield: 67%. Melting point: 174-175° C. (hexane-ethyl acetate).

¹H-NMR (CDCl₃) δ: 2.78 (2H, t, J=6.3 Hz), 3.71-3.79 (2H, m), 4.17 (2H, s), 6.59 (1H, br s), 6.84-6.90 (1H, m), 6.92-6.98 (1H, m), 7.03-7.06 (1H, m), 7.12-7.14 (1H, m), 7.29-7.33 (1H, m), 7.34-7.40 (1H, m), 7.54-7.61 (1H, m), 7.69-7.74 (1H, m), 7.75-7.84 (2H, m), 7.93-7.97 (1H, m).

### Working Example 170

### 3-[2-(3-Chloro-5-fluorobenzyl)-1-benzothiophen-4-yl]-N-(2-methoxyethyl)benzamide

A 3-[2-(3-chloro-5-fluorobenzyl)-1-benzothiophen-4-yl]benzoic acid crude product was synthesized from ethyl 3-[2-(3-chloro-5-fluorobenzyl)-1-benzothiophen-4-yl]benzoate obtained in Reference Example 199, and the resulting 3-[2-(3-chloro-5-fluorobenzyl)-1-benzothiophen-4-yl]benzoic acid crude product and 2-methoxyethanamine were used in the same manner as in Working Example 162 to obtain the titled compound. Yield: 67%. Oily substance.

¹H-NMR (CDCl₃) δ: 3.38 (3H, s), 3.54-3.61 (2H, m), 3.64-3.73 (2H, m), 4.17 (2H, s), 6.54 (1H, br s), 6.82-6.89 (1H, m), 6.92-6.98 (1H, m), 7.04 (1H, s), 7.15 (1H, d, J=0.8 Hz), 7.29-7.41 (2H, m), 7.55 (1H, t, J=7.7 Hz), 7.65-7.70 (1H, m), 7.74-7.78 (1H, m), 7.78-7.84 (1H, m), 7.92-7.97 (1H, m).

### Working Example 171

### N-(2-Amino-2-oxoethyl)-3-[2-(3-chloro-5-fluorobenzyl)-1-benzothiophen-4-yl]benzamide

A 3-[2-(3-chloro-5-fluorobenzyl)-1-benzothiophen-4-yl]benzoic acid crude product was synthesized from ethyl 3-[2-(3-chloro-5-fluorobenzyl)-1-benzothiophen-4-yl]benzoate obtained in Reference Example 199, and the resulting 3-[2-(3-chloro-5-fluorobenzyl)-1-benzothiophen-4-yl]benzoic acid crude product and glycinamide hydrochloride were used in the same manner as in Working Example 12 to obtain the titled compound. Yield: 72%. Melting point: 178-179° C. (hexane-ethyl acetate).

¹H-NMR (CDCl₃) δ: 4.17 (2H, s), 4.20 (2H, d, J=4.9 Hz), 5.46 (1H, br s), 5.94 (1H, br s), 6.83-6.90 (1H, m), 6.91-6.98 (2H, m), 7.04 (1H, s), 7.13 (1H, d, J=0.8 Hz), 7.29-7.40 (2H, m), 7.53-7.60 (1H, m), 7.68-7.73 (1H, m), 7.74-7.79 (1H, m), 7.82-7.87 (1H, m), 7.97-8.01 (1H, m).

### Working Example 172

### 3-[2-(3-Chloro-4-fluorobenzyl)-1-benzothiophen-7-yl]-N-(2-cyanoethyl)benzamide

A 3-[2-(3-chloro-4-fluorobenzyl)-1-benzothiophen-7-yl]benzoic acid crude product was synthesized from ethyl 3-[2-(3-chloro-4-fluorobenzyl)-1-benzothiophen-7-yl]benzoate obtained in Reference Example 204, and the resulting 3-[2-(3-chloro-4-fluorobenzyl)-1-benzothiophen-7-yl]benzoic acid crude product and 3-aminopropanenitrile were used in the same manner as in Working Example 162 to obtain the titled compound. Yield: 70%. Melting point: 104-105° C. (hexane-diethyl ether).

¹H-NMR (CDCl₃) δ: 2.73-2.80 (2H, m), 3.69-3.79 (2H, m), 4.16 (2H, s), 6.58 (1H, br s), 7.02-7.17 (3H, m), 7.27-7.34 (2H, m), 7.39-7.46 (1H, m), 7.56 (1H, t, J=7.7 Hz), 7.69 (1H, dd, J=8.0, 0.8 Hz), 7.77-7.87 (2H, m), 8.03-8.08 (1H, m).

### Working Example 173

### 3-[2-(3-Chloro-4-fluorobenzyl)-1-benzothiophen-7-yl]-N-(2-methoxyethyl)benzamide

A 3-[2-(3-chloro-4-fluorobenzyl)-1-benzothiophen-7-yl]benzoic acid crude product was synthesized from ethyl 3-[2-(3-chloro-4-fluorobenzyl)-1-benzothiophen-7-yl]benzoate obtained in Reference Example 204, and the resulting 3-[2-(3-chloro-4-fluorobenzyl)-1-benzothiophen-7-yl]benzoic acid crude product and 2-methoxyethanamine were used in the same manner as in Working Example 162 to obtain the titled compound. Yield: 82%. Melting point: 112-113° C. (hexane-ethyl acetate).

¹H-NMR (CDCl₃) δ: 3.37 (3H, s), 3.53-3.59 (2H, m), 3.63-3.71 (2H, m), 4.16 (2H, s), 6.53 (1H, br s), 7.02-7.17 (3H, m), 7.26-7.34 (2H, m), 7.39-7.46 (1H, m), 7.53 (1H, d, J=7.7 Hz), 7.66-7.71 (1H, m), 7.77-7.83 (2H, m), 8.02-8.07 (1H, m).

### Working Example 174

### N-(2-Amino-2-oxoethyl)-3-[2-(3-chloro-4-fluorobenzyl)-1-benzothiophen-7-yl]benzamide

A 3-[2-(3-chloro-4-fluorobenzyl)-1-benzothiophen-7-yl]benzoic acid crude product was synthesized from ethyl 3-[2-(3-chloro-4-fluorobenzyl)-1-benzothiophen-7-yl]benzoate obtained in Reference Example 204, and the resulting 3-[2-(3-chloro-4-fluorobenzyl)-1-benzothiophen-7-yl]benzoic acid crude product and glycinamide hydrochloride were used in the same manner as in Working Example 12 to obtain the titled compound. Yield: 83%. Melting point: 130-131° C. (hexane-ethyl acetate).

¹H-NMR (CDCl₃) δ: 4.16 (2H, s), 4.19 (2H, d, J=4.9 Hz), 5.47 (1, br s), 6.04 (1, br s), 6.98 (1H, br s), 7.02-7.19 (3H, m), 7.27-7.34 (2H, m), 7.39-7.45 (1H, m), 7.52-7.58 (1H, m), 7.68 (1H, dd, J=8.0, 1.1 Hz), 7.84 (2H, dd, J=7.7, 1.9 Hz), 8.08-8.12 (1H, m).

### Working Example 175

### N-(2-Cyanoethyl)-3-[2-(3,4-difluorobenzyl)-1-benzothiophen-7-yl]benzamide

A 3-[2-(3,4-difluorobenzyl)-1-benzothiophen-7-yl]benzoic acid crude product was synthesized from ethyl 3-[2-(3,4-difluorobenzyl)-1-benzothiophen-7-yl]benzoate obtained in Reference Example 205, and the resulting 3-[2-(3,4-difluorobenzyl)-1-benzothiophen-7-yl]benzoic acid crude product and 3-aminopropanenitrile were used in the same manner as in Working Example 162 to obtain the titled compound. Yield: 53%. Melting point: 133-134° C. (hexane-diethyl ether).

¹H-NMR (CDCl₃) δ: 2.76 (2H, t, J=6.2 Hz), 3.69-3.78 (2H, m), 4.16 (2H, s), 6.58 (1H, br s), 6.99 (1H, br s), 7.02-7.13 (3H, m), 7.31 (1H, dd, J=7.4, 1.1 Hz), 7.39-7.46 (1H, m), 7.55 (1H, t, J=7.7 Hz), 7.68 (1H, dd, J=7.7, 1.1 Hz), 7.77-7.76 (2H, m), 8.05 (1H, t, J=1.6 Hz).

### Working Example 176

### 3-[2-(3,4-difluorobenzyl)-1-benzothiophen-7-yl]-N-(2-methoxyethyl)benzamide

A 3-[2-(3,4-difluorobenzyl)-1-benzothiophen-7-yl]benzoic acid crude product was synthesized from ethyl 3-[2-(3,4-difluorobenzyl)-1-benzothiophen-7-yl]benzoate obtained in Reference Example 205, and the resulting 3-[2-(3,4-difluorobenzyl)-1-benzothiophen-7-yl]benzoic acid crude product and 2-methoxyethanamine were used in the same manner as in Working Example 162 to obtain the titled compound. Yield: 68%. Melting point: 98-104° C. (hexane-ethyl acetate).

¹H-NMR (CDCl₃) δ: 3.37 (3H, s), 3.53-3.60 (2H, m), 3.63-3.72 (2H, m), 4.16 (2H, s), 6.53 (1H, br s), 6.99 (1H, br s), 7.02-7.14 (3H, m), 7.29-7.34 (1H, m), 7.39-7.45 (1H, m), 7.50-7.56 (1H, m), 7.68 (1H, dd, J=8.0, 1.1 Hz), 7.77-7.84 (2H, m), 8.02-8.06 (1H, m).

### Working Example 177

### N-(2-Amino-2-oxoethyl)-3-[2-(3,4-difluorobenzyl)-1-benzothiophen-7-yl]benzamide

A 3-[2-(3,4-difluorobenzyl)-1-benzothiophen-7-yl]benzoic acid crude product was synthesized from ethyl 3-[2-(3,4-difluorobenzyl)-1-benzothiophen-7-yl]benzoate obtained in Reference Example 205, and the resulting 3-[2-(3,4-difluorobenzyl)-1-benzothiophen-7-yl]benzoic acid crude product and glycinamide hydrochloride were used in the same manner as in Working Example 12 to obtain the titled compound. Yield: 65%. Melting point: 131-132° C. (hexane-ethyl acetate).

¹H-NMR (CDCl₃) δ: 4.17 (2H, s), 4.19 (2H, d, J=4.9 Hz), 5.43 (1H, br s), 5.96 (1H, br s), 6.89-7.02 (2H, m), 7.02-7.14 (3H, m), 7.31 (1H, dd, J=7.4, 1.1 Hz), 7.39-7.45 (1H, m), 7.52-7.59 (1H, m), 7.68 (1H, dd, J=8.0, 1.1 Hz), 7.84 (2H, dd, J=7.7, 1.6 Hz), 8.10 (1H, t, J=1.9 Hz).

### Working Example 178

### 3-[2-(3-Chloro-5-fluorobenzyl)-1-benzothiophen-7-yl]-N-(2-cyanoethyl)benzamide

A 3-[2-(3-chloro-5-fluorobenzyl)-1-benzothiophen-7-yl]benzoic acid crude product was synthesized from ethyl 3-[2-(3-chloro-5-fluorobenzyl)-1-benzothiophen-7-yl]benzoate obtained in Reference Example 206, and the resulting 3-[2-(3-chloro-5-fluorobenzyl)-1-benzothiophen-7-yl]benzoic acid crude product and 3-aminopropanenitrile were used in the same manner as in Working Example 162 to obtain the titled compound. Yield: 59%. Melting point: 116-117° C. (hexane-ethyl acetate).

¹H-NMR (CDCl₃) δ: 2.77 (2H, t, J=6.2 Hz), 3.70-3.79 (2H, m), 4.18 (2H, s), 6.58 (1H, br s), 6.85-6.92 (1H, m), 6.93-6.99 (1H, m), 7.05-7.08 (1H, m), 7.12-7.14 (1H, m), 7.31-7.35 (1H, m), 7.40-7.47 (1H, m), 7.57 (1H, t, J=7.7 Hz), 7.68-7.73 (1H, m), 7.78-7.82 (1H, m), 7.83-7.88 (1H, m), 8.05-8.07 (1H, m).

### Working Example 179

### 3-[2-(3-Chloro-5-fluorobenzyl)-1-benzothiophen-7-yl]-N-(2-methoxyethyl)benzamide

A 3-[2-(3-chloro-5-fluorobenzyl)-1-benzothiophen-7-yl]benzoic acid crude product was synthesized from ethyl 3-[2-(3-chloro-5-fluorobenzyl)-1-benzothiophen-7-yl]benzoate obtained in Reference Example 206, and the resulting 3-[2-(3-chloro-5-fluorobenzyl)-1-benzothiophen-7-yl]benzoic acid crude product and 2-methoxyethanamine were used in the same manner as in Working Example 162 to obtain the titled compound. Yield: 63%. Melting point: 94-95° C. (hexane-diethyl ether).

¹H-NMR (CDCl₃) δ: 3.37 (3H, s), 3.53-3.60 (2H, m), 3.63-3.71 (2H, m), 4.17 (2H, s), 6.53 (1H, br s), 6.85-6.91 (1H, m), 6.93-6.99 (1H, m), 7.04-7.07 (1H, m), 7.12-7.14 (1H, m), 7.31-7.35 (1H, m), 7.39-7.47 (1H, m), 7.54 (1H, t, J=7.4 Hz), 7.70 (1H, dd, J=7.7, 1.1 Hz), 7.78-7.85 (2H, m), 8.03-8.07 (1H, m).

### Working Example 180

### N-(2-Amino-2-oxoethyl)-3-[2-(3-chloro-5-fluorobenzyl)-1-benzothiophen-7-yl]benzamide

A 3-[2-(3-chloro-5-fluorobenzyl)-1-benzothiophen-7-yl]benzoic acid crude product was synthesized from ethyl 3-[2-(3-chloro-5-fluorobenzyl)-1-benzothiophen-7-yl]benzoate obtained in Reference Example 206, and the resulting 3-[2-(3-chloro-5-fluorobenzyl)-1-benzothiophen-7-yl]benzoic acid crude product and glycinamide hydrochloride were used in the same manner as in Working Example 12 to obtain the titled compound. Yield: 68%. Melting point: 165-166° C. (hexane-ethyl acetate).

¹H-NMR (CDCl₃) δ: 4.15-4.22 (4H, m), 5.46 (1H, s), 6.02 (1H, br s), 6.84-7.00 (3H, m), 7.06 (1H, s), 7.12 (1H, s), 7.30-7.35 (1H, m), 7.43 (1H, t, J=7.4 Hz), 7.52-7.60 (1H, m), 7.70 (1H, dd, J=7.7, 0.8 Hz), 7.81-7.88 (2H, m), 8.09-8.13 (1H, m).

### Working Example 181

### N-(2-Hydroxyethyl)-3-(2-[[3-(trifluoromethyl)phenoxy]methyl]-1-benzothiophen-7-yl)benzamide

A 3-(2-[[3-(trifluoromethyl)phenoxy]methyl]-1-benzothiophen-7-yl]benzoic acid crude product was synthesized from ethyl 3-(2-[[3-(trifluoromethyl)phenoxy]methyl]-1-benzothiophen-7-yl]benzoate obtained in Reference Example 228, and the resulting 3-(2-[[3-(trifluoromethyl)phenoxy]methyl]-1-benzothiophen-7-yl]benzoic acid crude product and 2-aminoethanol were used in the same manner as in Working Example 164 to obtain the titled compound. Yield: 71%. Oily substance.

¹H-NMR (CDCl₃) δ: 2.50 (1H, t, J=4.9 Hz), 3.63-3.71 (2H, m), 3.86 (2H, q, J=5.1 Hz), 5.35 (2H, s), 6.67 (1H, br s), 7.13-7.19 (1H, m), 7.21-7.26 (2H, m), 7.35-7.52 (4H, m), 7.54-7.61 (1H, m), 7.77 (1H, dd, J=7.8, 1.2 Hz), 7.85 (2H, tt, J=7.9, 1.4 Hz), 8.09 (1H, t, J=1.8 Hz).

### Working Example 182

### N-(2-Methoxyethyl)-3-(2-[[3-(trifluoromethyl)phenoxy]methyl]-1-benzothiophen-7-yl)benzamide

A 3-(2-[[3-(trifluoromethyl)phenoxy]methyl]-1-benzothiophen-7-yl]benzoic acid crude product was synthesized from ethyl 3-(2-[[3-(trifluoromethyl)phenoxy]methyl]-1-benzothiophen-7-yl]benzoate obtained in Reference Example 228, and the resulting 3-(2-[[3-(trifluoromethyl)phenoxy]methyl]-1-benzothiophen-7-yl]benzoic acid crude product and 2-methoxyethanamine were used in the same manner as in Working Example 162 to obtain the titled compound. Yield: 77%. Oily substance.

¹H-NMR (CDCl₃) δ: 3.39 (3H, s), 3.55-3.61 (2H, m), 3.65-3.73 (2H, m), 5.35 (2H, s), 6.57 (1H, br s), 7.16 (1H, dd, J=8.0, 2.5 Hz), 7.21-7.25 (2H, m), 7.36-7.44 (3H, m), 7.45-7.51 (1H, m), 7.54-7.61 (1H, m), 7.77 (1H, dd, J=7.8, 1.2 Hz), 7.81-7.89 (2H, m), 8.09 (1H, t, J=1.5 Hz).

### Working Example 183

### N-(2-Amino-2-oxoethyl)-3-(2-[[3-(trifluoromethyl)phenoxy]methyl]-1-benzothiophen-7-yl)benzamide

A 3-(2-[[3-(trifluoromethyl)phenoxy]methyl]-1-benzothiophen-7-yl]benzoic acid crude product was synthesized from ethyl 3-(2-[[3-(trifluoromethyl)phenoxy]methyl]-1-benzothiophen-7-yl]benzoate obtained in Reference Example 228, and the resulting 3-(2-[[3-(trifluoromethyl)phenoxy]methyl]-1-benzothiophen-7-yl]benzoic acid crude product and glycinamide hydrochloride were used in the same manner as in Working Example 12 to obtain the titled compound. Yield: 43%. Melting point: 132-133° C. (hexane-ethyl acetate).

¹H-NMR (CDCl₃) δ: 4.20 (2H, d, J=4.9 Hz), 5.35 (2H, s), 6.99 (1H, br s), 7.12-7.20 (1H, m), 7.21-7.25 (2H, m), 7.35-7.44 (3H, m), 7.44-7.51 (1H, m), 7.55-7.62 (1H, m), 7.77 (1H, d, J=8.0 Hz), 7.83-7.93 (2H, m), 8.14 (1H, t, J=1.8 Hz), 2H, unconfirmed.

### Working Example 184

### 3-(2-[[3-(Trifluoromethyl)phenoxy]methyl]-1-benzothiophen-7-yl]benzamide

A 3-(2-[[3-(trifluoromethyl)phenoxy]methyl]-1-benzothiophen-7-yl]benzoic acid crude product was synthesized from ethyl 3-(2-[[3-(trifluoromethyl)phenoxy]methyl]-1-benzothiophen-7-yl]benzoate obtained in Reference Example 228, and the resulting 3-(2-[[3-(trifluoromethyl)phenoxy]methyl]-1-benzothiophen-7-yl]benzoic acid crude product and 28% aqueous ammonia were used in the same manner as in Working Example 157 to obtain the titled compound. Yield: 52%. Melting point: 79-80° C. (hexane-ethyl acetate-diethyl ether).

¹H-NMR (CDCl₃) δ: 5.35 (2H, s), 7.16 (1H, dd, J=8.0, 2.2 Hz), 7.21-7.25 (2H, m), 7.36-7.44 (3H, m), 7.44-7.51 (1H, m), 7.56-7.63 (1H, m), 7.78 (1H, dd, J=7.8, 1.0 Hz), 7.85-7.92 (2H, m), 8.13 (1H, t, J=1.8 Hz), 2H, unconfirmed.

### Working Example 185

### N-(2-Amino-2-oxoethyl)-3-[2-[(3-chloro-5-fluorophenoxy)methyl]-1-benzothiophen-7-yl]benzamide

A 3-[2-[(3-chloro-5-fluorophenoxy)methyl]-1-benzothiophen-7-yl]benzoic acid crude product was synthesized from ethyl 3-[2-[(3-chloro-5-fluorophenoxy)methyl]-1-benzothiophen-7-yl]benzoate obtained in Reference Example 229, and the resulting 3-[2-[(3-chloro-5-fluorophenoxy)methyl]-1-benzothiophen-7-yl]benzoic acid crude product and glycinamide hydrochloride were used in the same manner as in Working Example 12 to obtain the titled compound. Yield: 51%. Melting point: 158-159° C. (hexane-ethyl acetate).

¹H-NMR (CDCl₃) δ: 4.21 (2H, d, J=4.9 Hz), 5.28 (2H, s), 5.49 (1H, br s), 6.10 (1H, br s), 6.62 (1H, dt, J=10.3, 2.1 Hz), 6.72 (1H, dt, J=8.3, 1.8 Hz), 6.77-6.82 (1H, m), 7.04 (1H, br s), 7.35-7.42 (2H, m), 7.43-7.50 (1H, m), 7.54-7.62 (1H, m), 7.77 (1H, dd, J=8.0, 1.1 Hz), 7.87 (2H, dt, J=7.7, 1.5 Hz), 8.14 (1H, t, J=1.8 Hz).

### Working Example 186

### 3-[2-[(3-Chloro-5-fluorophenoxy)methyl]-1-benzothiophen-7-yl]benzamide

A 3-[2-[(3-chloro-5-fluorophenoxy)methyl]-1-benzothiophen-7-yl]benzoic acid crude product was synthesized from ethyl 3-[2-[(3-chloro-5-fluorophenoxy)methyl]-1-benzothiophen-7-yl]benzoate obtained in Reference Example 229, and the resulting 3-[2-[(3-chloro-5-fluorophenoxy)methyl]-1-benzothiophen-7-yl]benzoic acid crude product and aqueous ammonia were used in the same manner as in Working Example 157 to obtain the titled compound. Yield: 44%. Melting point: 110-111° C. (hexane-ethyl acetate).

¹H-NMR (CDCl₃) δ: 5.29 (2H, s), 6.58-6.65 (1H, m), 6.69-6.76 (1H, m), 6.80 (1H, s), 7.37-7.43 (2H, m), 7.44-7.51 (1H, m), 7.56-7.64 (1H, m), 7.75-7.81 (1H, m), 7.85-7.92 (2H, m), 8.10-8.15 (1H, m), 2H, unconfirmed.

### Working Example 187

### 3-[2-[(3-Chloro-5-fluorophenoxy)methyl]-1-benzothiophen-7-yl]-N-(2-methoxyethyl)benzamide

A 3-[2-[(3-chloro-5-fluorophenoxy)methyl]-1-benzothiophen-7-yl]benzoic acid crude product was synthesized from ethyl 3-[2-[(3-chloro-5-fluorophenoxy)methyl]-1-benzothiophen-7-yl]benzoate obtained in Reference Example 229, and the resulting 3-[2-[(3-chloro-5-fluorophenoxy)methyl]-1-benzothiophen-7-yl]benzoic acid crude product and 2-methoxyethanamine were used in the same manner as in Working Example 162 to obtain the titled compound. Yield: 61%. Oily substance.

¹H-NMR (CDCl₃) δ: 3.38 (3H, s), 3.53-3.62 (2H, m), 3.64-3.73 (2H, m), 5.27 (2H, s), 6.56 (1H, br s), 6.61 (1H, dt, J=10.2, 2.2 Hz), 6.71 (1H, dt, J=8.5, 2.0 Hz), 6.76-6.82 (1H, m), 7.34-7.43 (2H, m), 7.43-7.51 (1H, m), 7.53-7.61 (1H, m), 7.73-7.89 (3H, m), 8.08 (1H, t, J=1.5 Hz).

### Working Example 188

### 3-Fluoro-N-(2-methoxyethyl)-5-[2-[[3-(trifluoromethyl)phenoxy]methyl]-1-benzothiophen-7-yl)benzamide

A 3-fluoro-5-[2-[[3-(trifluoromethyl)phenoxy]methyl]-1-benzothiophen-7-yl]benzoic acid crude product was synthesized from ethyl 3-fluoro-5-[2-[[3-(trifluoromethyl)phenoxy]methyl]-1-benzothiophen-7-yl]benzoate obtained in Reference Example 232, and the resulting 3-fluoro-5-[2-[[3-(trifluoromethyl)phenoxy]methyl]-1-benzothiophen-7-yl]benzoic acid crude product and 2-methoxyethanamine were used in the same manner as in Working Example 162 to obtain the titled compound. Yield: 95%. Oily substance.

¹H-NMR (CDCl₃) δ: 3.38 (3H, s), 3.53-3.62 (2H, m), 3.68 (2H, q, J=5.0 Hz), 5.36 (2H, s), 6.53 (1H, br s), 7.10-7.25 (3H, m), 7.34-7.42 (2H, m), 7.43 (1H, s), 7.48 (1H, t, J=7.5 Hz), 7.52-7.60 (2H, m), 7.79 (1H, dd, J=7.8, 1.0 Hz), 7.86 (1H, t, J=1.5 Hz).

### Working Example 189

### N-(2-Amino-2-oxoethyl)-3-fluoro-5-[2-[[3-(trifluoromethyl)phenoxy]methyl]-1-benzothiophen-7-yl]benzamide

A 3-fluoro-5-[2-[[3-(trifluoromethyl)phenoxy]methyl]-1-benzothiophen-7-yl]benzoic acid crude product was synthesized from ethyl 3-fluoro-5-[2-[[3-(trifluoromethyl)phenoxy]methyl]-1-benzothiophen-7-yl]benzoate obtained in Reference Example 232, and the resulting 3-fluoro-5-[2-[[3-(trifluoromethyl)phenoxy]methyl]-1-benzothiophen-7-yl]benzoic acid crude product and glycinamide hydrochloride were used in the same manner as in Working Example 12 to obtain the titled compound. Yield: 87%. Melting point: 162-163° C. (hexane-ethyl acetate).

¹H-NMR (CDCl₃) δ: 4.20 (2H, d, J=4.9 Hz), 5.35 (2H, s), 5.52 (1H, br s), 6.01 (1H, br s), 7.06 (1H, br s), 7.16 (1H, dt, J=8.7, 2.1 Hz), 7.21-7.26 (2H, m), 7.34-7.53 (4H, m), 7.54-7.65 (2H, m), 7.79 (1H, dd, J=8.0, 1.1 Hz), 7.92 (1H, t, J=1.4 Hz).

### Working Example 190

### N-(2-Methoxyethyl)-3-[2-[(5-methoxypyridin-3-yl)methyl]-1-benzothiophen-7-yl]benzamide hydrochloride

4 N sodium hydroxide aqueous solution (1.0 mL, 4.0 mmol) was added to a mixture of ethyl 3-[2-[(5-methoxypyridin-3-yl)methyl]-1-benzothiophen-7-yl]benzoate (0.34 g, 0.84 mmol) obtained in Reference Example 258, ethanol (8 mL), and THF (3 mL), and the mixture was stirred for 2 hours at room temperature and then stirred for 2 hours at 60° C. The reaction solution was neutralized with the addition of 1 N hydrochloric acid (4.0 mL), and the solvent was distilled off at reduced pressure. A mixture of the residue, 2-methoxyethanamine (95 mg, 1.3 mmol), WSC (320 mg, 1.7 mmol), HOBt (260 mg, 1.7 mmol), and triethylamine (430 mg, 4.2 mmol) in DMF (5 mL) was stirred for 15 hours at room temperature. The reaction solution was diluted with water and extracted with ethyl acetate. The extract was washed with saturated brine and dried over anhydrous magnesium sulfate, and the solvent was then distilled off at reduced pressure. The residue was purified by silica gel column chromatography (hexane:ethyl acetate=90:10→0/100). An ethyl acetate solution (0.1 mL) of 4 N hydrogen chloride was added to an ethyl acetate (2.0 mL) solution of the resulting pale yellow oily substance, the mixture was stirred for 5 min, and the solvent was then distilled off at reduced pressure to give 0.02 g of the titled compound (yield 5%). Amorphous solids.

¹H-NMR (DMSO-d₆) δ: 3.27 (3H, s), 3.37-3.54 (4H, m), 3.89 (3H, s), 4.39 (2H, s), 7.36-7.45 (1H, m), 7.46-7.54 (1H, m) 7.57-7.65 (1 H, m), 7.77-7.87 (3H, m), 7.88-7.95 (1H, m), 8.11-8.16 (1H, m), 8.37-8.45 (2H, m), 8.65 (1H, br s).

### Working Example 191

### N-(2-Hydroxyethyl)-3-[3-methyl-2-[3-(trifluoromethyl)benzyl]-1-benzothiophen-7-yl]benzamide

3-[3-Methyl-2-[3-(trifluoromethyl)benzyl]-1-benzothiophen-7-yl]benzoic acid obtained in Reference Example 237 and 2-aminoethanol were used in the same manner as in Working Example 22 to obtain the titled compound. Yield: 84%; melting point: 140-145° C. (ethyl acetate-hexane).

¹H-NMR (CDCl₃) δ: 2.41 (3H, s), 2.42-2.47 (1H, m), 3.60-3.69 (2H, m), 3.84 (2H, q, J=4.8 Hz), 4.26 (2H, s), 6.62 (1H, br s), 7.35 (1H, dd, J=7.2, 1.1 Hz), 7.37-7.41 (2H, m), 7.44-7.57 (4H, m), 7.68 (1H, dd, J=8.0, 1.1 Hz), 7.76-7.87 (2H, m), 8.05 (1H, s).

### Working Example 192

### N-(2-Cyanoethyl)-3-[3-methyl-2-[3-(trifluoromethyl)benzyl]-1-benzothiophen-7-yl]benzamide

3-[3-Methyl-2-[3-(trifluoromethyl)benzyl]-1-benzothiophen-7-yl]benzoic acid obtained in Reference Example 237 and 3-aminopropanenitrile were used in the same manner as in Working Example 3 to obtain the titled compound. Yield: 80%; melting point: 135-136° C. (ethyl acetate-hexane).

¹H-NMR (CDCl₃) δ: 2.42 (3H, s), 2.75 (2H, t, J=6.2 Hz), 3.73 (2H, q, J=6.1 Hz), 4.26 (2H, s), 6.60 (1H, br s), 7.36 (1H, d, J=7.2 Hz), 7.37-7.41 (2H, m), 7.42-7.52 (3H, m), 7.56 (1H, t, J=7.8 Hz), 7.69 (1H, d, J=8.0 Hz), 7.80 (1H, d, J=8.0 Hz), 7.85 (1H, d, J=7.6 Hz), 8.06 (1H, s).

### Working Example 193

### N-(2-Methoxyethyl)-3-[3-methyl-2-[3-(trifluoromethyl)benzyl]-1-benzothiophen-7-yl]benzamide

3-[3-Methyl-2-[3-(trifluoromethyl)benzyl]-1-benzothiophen-7-yl]benzoic acid obtained in Reference Example 237 and 2-methoxyethanamine were used in the same manner as in Working Example 3 to obtain the titled compound. Yield: 88%; melting point: 135-136° C. (ethyl acetate-hexane).

¹H-NMR (CDCl₃) δ: 2.41 (3H, s), 3.36 (3H, s), 3.51-3.61 (2H, m), 3.67 (2H, q, J=5.0 Hz), 4.26 (2H, s), 6.54 (1H, br s), 7.33-7.41 (3H, m), 7.43-7.59 (4H, m), 7.68 (1H, dd, J=8.1, 1.1 Hz), 7.77-7.86 (2H, m), 8.05 (1H, t, J=1.7 Hz).

### Working Example 194

### 3-[3-Methyl-2-[3-(trifluoromethyl)benzyl]-1-benzothiophen-7-yl]-N-[2-(propan-2-yloxy)ethyl]benzamide

3-[3-Methyl-2-[3-(trifluoromethyl)benzyl]-1-benzothiophen-7-yl]benzoic acid obtained in Reference Example 237 and 2-(propan-2-yloxy)ethanamine were used in the same manner as in Working Example 3 to obtain the titled compound. Yield: 91%; melting point: 121-122° C. (ethyl acetate-hexane).

¹H-NMR (CDCl₃) δ: 1.15 (6H, d, J=6.1 Hz), 2.41 (3H, s), 3.55-3.67 (5H, m), 4.26 (2H, s), 6.60 (1H, br s), 7.33-7.41 (3H, m), 7.43-7.57 (4H, m), 7.68 (1H, d, J=8.0 Hz), 7.76-7.86 (2H, m), 8.06 (1H, s).

### Working Example 195

### N-(2-tert-butoxyethyl)-3-[3-methyl-2-[3-(trifluoromethyl)benzyl]-1-benzothiophen-7-yl]benzamide

3-[3-Methyl-2-[3-(trifluoromethyl)benzyl]-1-benzothiophen-7-yl]benzoic acid obtained in Reference Example 237 and 2-tert-butoxyethanamine were used in the same manner as in Working Example 3 to obtain the titled compound. Yield: 74%, amorphous solids.

¹H-NMR (CDCl₃) δ: 1.18 (9H, s), 2.41 (3H, s), 3.50-3.58 (2H, m), 3.58-3.66 (2H, m), 4.26 (2H, s), 6.62 (1H, br s), 7.34-7.40 (3H, m), 7.43-7.57 (4H, m), 7.68 (1H, d, J=6.8 Hz), 7.76-7.84 (2H, m), 8.06 (1H, s).

### Working Example 196

### N-(2-Amino-2-oxoethyl)-3-[3-methyl-2-[3-(trifluoromethyl)benzyl]-1-benzothiophen-7-yl]benzamide

Glycinamide hydrochloride (61.9 mg, 0.56 mmol) and triethylamine (98.8 μL, 0.71 mmol) were added to a DMF (2 mL) solution of 3-[3-methyl-2-[3-(trifluoromethyl)benzyl]-1-benzothiophen-7-yl]benzoic acid (200 mg, 0.47 mmol) obtained in Reference Example 237, WSC (107 mg, 0.56 mmol), and HOBt (75.7 mg, 0.56 mmol), and the mixture was stirred for 16 hours at room temperature. The addition of saturated sodium bicarbonate aqueous solution to the reaction solution was followed by extraction with ethyl acetate. The combined organic layers were washed with water, dried over anhydrous magnesium sulfate, and then concentrated at reduced pressure. The residue was purified by basic silica gel column chromatography (ethyl acetate-methanol 70:30→0:100) and recrystallized from hexane and THF to give 190 mg of the titled compound (yield 84%). Melting point: 187-188° C.

¹H-NMR (CDCl₃) δ: 2.43 (3H, s), 3.82 (2H, d, J=5.8 Hz), 4.38 (2H, s), 7.04 (1H, br s), 7.38 (1H, br s), 7.42-7.49 (1H, m), 7.49-7.68 (6H, m), 7.77 (1H, d, J=7.3 Hz), 7.83 (1H, d, J=7.7 Hz), 7.93 (1H, d, J=7.9 Hz), 8.14 (1H, s), 8.79 (1H, t, J=5.8 Hz).

### Working Example 197

### 3-[3-Methyl-2-[3-(trifluoromethyl)benzyl]-1-benzothiophen-7-yl]benzamide

A DMF (2 mL) solution of 3-[3-methyl-2-[3-(trifluoromethyl)benzyl]-1-benzothiophen-7-yl]benzoic acid (200 mg, 0.47 mmol) obtained in Reference Example 237, WSC (107 mg, 0.56 mmol), and HOBt (75.7 mg, 0.56 mmol) was stirred for 5 hours at room temperature, and the reaction solution was then poured into 28% aqueous ammonia and extracted with ethyl acetate. The combined organic layers were washed with water, dried over anhydrous magnesium sulfate, and then concentrated at reduced pressure. The residue was purified by basic silica gel column chromatography (ethyl acetate-methanol 90:10→50:50) and recrystallized from hexane and ethyl acetate to give 120 mg of the titled compound (yield 60%). Melting point: 190-191° C.

¹H-NMR (CDCl₃) δ: 2.42 (3H, s), 4.26 (2H, s), 5.63 (1H, br s), 6.07 (1H, br s), 7.22-7.30 (1H, m), 7.34-7.51 (5H, m), 7.56 (1H, t, J=7.7 Hz), 7.69 (1H, d, J=8.1 Hz), 7.80-7.92 (2H, m), 8.05-8.13 (1H, m).

### Working Example 198

### N-(2-Hydroxyethyl)-2-[2-[3-(trifluoromethyl)benzyl]-1-benzothiophen-7-yl]pyridine-4-carboxamide

2-[2-[3-(Trifluoromethyl)benzyl]-1-benzothiophen-7-yl]pyridine-4-carboxylic acid obtained in Reference Example 238 and 2-aminoethanol were used in the same manner as in Working Example 22 to obtain the titled compound. Yield: 77%; melting point: 168-169° C. (ethyl acetate-hexane).

¹H-NMR (CDCl₃) δ: 2.17 (1H, t, J=4.7 Hz), 3.69 (2H, q, J=5.0 Hz), 3.89 (2H, q, J=4.9 Hz), 4.32 (2H, s), 6.76 (1H, br s), 7.10 (1H, s), 7.37-7.56 (5H, m), 7.58 (1H, s), 7.79 (1H, d, J=8.0 Hz), 7.91 (1H, d, J=6.8 Hz), 8.30 (1H, s), 8.89 (1H, d, J=4.5 Hz).

### Working Example 199

### N-(2-Methoxyethyl)-2-[2-[3-(trifluoromethyl)benzyl]-1-benzothiophen-7-yl]pyridine-4-carboxamide

2-[2-[3-(Trifluoromethyl)benzyl]-1-benzothiophen-7-yl]pyridine-4-carboxylic acid obtained in Reference Example 238 and 2-methoxyethanamine were used in the same manner as in Working Example 3 to obtain the titled compound. Yield: 65%; melting point: 108-109° C. (ethyl acetate-hexane).

¹H-NMR (CDCl₃ ) δ: 3.41 (3H, s), 3.56-3.64 (2H, m), 3.71 (2H, q, J=5.2 Hz), 4.32 (2H, s), 6.64 (1H, br s), 7.10 (1H, s), 7.40-7.55 (5H, m), 7.58 (1H, s), 7.79 (1H, d, J=8.0 Hz), 7.92 (1H, d, J=6.8 Hz), 8.30 (1H, s), 8.89 (1H, d, J=4.9 Hz).

### Working Example 200

### N-(2-Amino-2-oxoethyl)-2-[2-[3-(trifluoromethyl)benzyl]-1-benzothiophen-7-yl]pyridine-4-carboxamide

2-[2-[3-(Trifluoromethyl)benzyl]-1-benzothiophen-7-yl]pyridine-4-carboxylic acid obtained in Reference Example 238 and glycinamide hydrochloride were used in the same manner as in Working Example 196 to obtain the titled compound. Yield: 62%; melting point: 176-177° C. (ethyl acetate-hexane).

¹H-NMR (CDCl₃) δ: 4.22 (2H, d, J=4.9 Hz), 4.32 (2H, s), 5.55 (1H, br s), 5.89 (1H, br s), 7.09 (1H, s), 7.19 (1H, br s), 7.37-7.54 (4H, m), 7.55-7.62 (2H, m), 7.78 (1H, d, J=8.0 Hz), 7.91 (1H, d, J=7.6 Hz), 8.33 (1H, s), 8.91 (1H, d, J=4.9 Hz).

### Working Example 201

### 2-[2-[3-(Trifluoromethyl)benzyl]-1-benzothiophen-7-yl]pyridine-4-carboxamide

2-[2-[3-(Trifluoromethyl)benzyl]-1-benzothiophen-7-yl]pyridine-4-carboxylic acid obtained in Reference Example 238 was used in the same manner as in Working Example 197 to obtain the titled compound. Yield: 51%; melting point: 177-178° C. (ethyl acetate-hexane).

¹H-NMR (CDCl₃ ) δ: 4.32 (2H, s), 5.76 (1H, br s), 6.18 (1H, br s), 7.10 (1H, s), 7.40-7.56 (5H, m), 7.58 (1H, s), 7.79 (1H, d, J=6.8 Hz), 7.92 (1H, d, J=7.6 Hz), 8.33 (1H, s), 8.92 (1H, d, J=6.1 Hz).

### Working Example 202

### 2-[2-(3-Chloro-5-fluorobenzyl)-1-benzothiophen-7-yl]-N-(2-hydroxyethyl)pyridine-4-carboxamide

2-[2-(3-Chloro-5-fluorobenzyl)-1-benzothiophen-7-yl]pyridine-4-carboxylic acid obtained in Reference Example 239 and 2-aminoethanol were used in the same manner as in Working Example 22 to obtain the titled compound. Yield: 73%; melting point: 172-173° C. (ethyl acetate-hexane).

¹H-NMR (CDCl₃) δ: 2.14 (1H, t, J=4.9 Hz), 3.63-3.77 (2H, m), 3.89 (2H, q, J=4.9 Hz), 4.22 (2H, s), 6.75 (1H, br s), 6.89-7.02 (2H, m), 7.07-7.18 (2H, m), 7.44-7.51 (1H, m), 7.54 (1H, d, J=4.9 Hz), 7.80 (1H, d, J=8.0 Hz), 7.93 (1H, d, J=7.6 Hz), 8.31 (1H, s), 8.91 (1H, d, J=4.9 Hz).

### Working Example 203

### 2-[2-(3-Chloro-5-fluorobenzyl)-1-benzothiophen-7-yl]pyridine-4-carboxamide

2-[2-(3-Chloro-5-fluorobenzyl)-1-benzothiophen-7-yl]pyridine-4-carboxylic acid obtained in Reference Example 239 was used in the same manner as in Working Example 197 to obtain the titled compound. Yield: 70%; melting point: 173-175° C. (ethyl acetate-hexane).

¹H-NMR (CDCl₃) δ: 4.23 (2H, s), 5.80 (1H, br s), 6.21 (1H, br s), 6.88-7.01 (2H, m), 7.11 (1H, s), 7.13 (1H, s), 7.44-7.52 (1H, m), 7.55 (1H, dd, J=4.9, 1.5 Hz), 7.81 (1H, d, J=6.8 Hz), 7.93 (1H, d, J=6.4 Hz), 8.34 (1H, s), 8.93 (1H, d, J=5.3 Hz).

### Working Example 204

### N-(2-Hydroxyethyl)-2-[3-methyl-2-[3-(trifluoromethyl)benzyl]-1-benzothiophen-7-yl]pyridine-4-carboxamide

2-[3-Methyl-2-[3-(trifluoromethyl)benzyl]-1-benzothiophen-7-yl]pyridine-4-carboxylic acid obtained in Reference Example 240 and 2-aminoethanol were used in the same manner as in Working Example 22 to obtain the titled compound. Yield: 61%; melting point: 201-202° C. (THF-hexane).

¹H-NMR (CDCl₃) δ:2.08 (1H, t, J=4.9 Hz), 2.41 (3H, s), 3.70 (2H, q, J=5.0 Hz), 3.89 (2H, q, J=4.8 Hz), 4.31 (2H, s), 6.70 (1H, br s), 7.36-7.48 (3H, m), 7.49-7.57 (3H, m), 7.78 (1H, d, J=7.2 Hz), 7.95 (1H, d, J=7.2 Hz), 8.30 (1H, s), 8.90 (1H, d, J=4.9 Hz).

### Working Example 205

### 2-[3-Methyl-2-[3-(trifluoromethyl)benzyl]-1-benzothiophen-7-yl]pyridine-4-carboxamide

2-[3-Methyl-2-[3-(trifluoromethyl)benzyl]-1-benzothiophen-7-yl]pyridine-4-carboxylic acid obtained in Reference Example 240 was used in the same manner as in Working Example 197 to obtain the titled compound. Yield: 17%; melting point: 193-194° C. (ethyl acetate-hexane).

¹H-NMR (CDCl₃) δ: 2.41 (3H, s), 4.31 (2H, s), 5.77 (1H, br s), 6.19 (1H, br s), 7.34-7.48 (3H, m), 7.50-7.57 (3H, m), 7.78 (1H, dd, J=7.9, 0.8 Hz), 7.95 (1H, dd, J=7.5, 0.8 Hz), 8.33 (1H, s), 8.92 (1H, dd, J=5.3, 0.8 Hz).

### Working Example 206

### 2-[2-[3-(Trifluoromethyl)benzyl]-1-benzothiophen-7-yl]benzamide

7-Bromo-2-[3-(trifluoromethyl)benzyl]-1-benzothiophene obtained in Reference Example 190 and (2-carbamoylphenyl)boronic acid were used in the same manner as in Working Example 9 to obtain the titled compound. Yield: 36%, amorphous solids.

¹H-NMR (CDCl₃) δ: 4.24 (2H, s), 5.22 (2H, br s), 7.08 (1H, s), 7.22-7.28 (1H, m), 7.36-7.56 (8H, m), 7.70 (1H, dd, J=8.1, 0.9 Hz), 7.87-7.98 (1H, m).

### Working Example 207

### 4-[2-[3-(Trifluoromethyl)benzyl]-1-benzothiophen-7-yl]benzamide

7-Bromo-2-[3-(trifluoromethyl)benzyl]-1-benzothiophene obtained in Reference Example 190 and (4-carbamoylphenyl)boronic acid were used in the same manner as in Working Example 9 to obtain the titled compound. Yield: 68%; melting point: 175-176° C. (ethyl acetate-hexane).

¹H-NMR (CDCl₃ ) δ: 4.28 (2H, s), 5.66 (1H, br s), 6.07 (1H, br s), 7.12 (1H, s), 7.33 (1H, d, J=6.4 Hz), 7.40-7.49 (3H, m), 7.52 (2H, d, J=11.7 Hz), 7.71 (1H, d, J=7.2 Hz), 7.77 (2H, d, J=8.3 Hz), 7.92 (2H, d, J=8.3 Hz).

### Working Example 208

### N-(2-Hydroxyethyl)-4-[2-[3-(trifluoromethyl)benzyl]-1-benzothiophen-7-yl]benzamide

4-[2-[3-(Trifluoromethyl)benzyl]-1-benzothiophen-7-yl]benzoic acid obtained in Reference Example 241 and 2-aminoethanol were used in the same manner as in Working Example 22 to obtain the titled compound. Yield: 76%; melting point: 120-121° C. (ethyl acetate-hexane).

¹H-NMR (CDCl₃) δ: 2.44 (1H, t, J=4.9 Hz), 3.61-3.74 (2H, m), 3.87 (2H, q, J=5.0 Hz), 4.27 (2H, s), 6.64 (1H, br s), 7.12 (1H, s), 7.32 (1H, dd, J=7.3, 0.9 Hz), 7.40-7.48 (3H, m), 7.52 (2H, d, J=11.3 Hz), 7.70 (1H, dd, J=7.9, 1.1 Hz), 7.73-7.79 (2H, m), 7.85-7.95 (2H, m).

### Working Example 209

### N-(2-Amino-2-oxoethyl)-4-[2-[3-(trifluoromethyl)benzyl]-1-benzothiophen-7-yl]benzamide

4-[2-[3-(Trifluoromethyl)benzyl]-1-benzothiophen-7-yl]benzoic acid obtained in Reference Example 241 and glycinamide hydrochloride were used in the same manner as in Working Example 196 to obtain the titled compound. Yield: 77%; melting point: 134-136° C. (ethyl acetate-hexane).

¹H-NMR (CDCl₃ ) δ: 4.22 (2H, d, J=5.3 Hz), 4.26 (2H, s), 5.51 (1H, br s), 6.14 (1H, br s), 7.05 (1H, t, J=4.5 Hz), 7.11 (1H, s), 7.29-7.35 (1H, m), 7.38-7.48 (3H, m), 7.49-7.57 (2H, m), 7.70 (1H, dd, J=7.9, 1.1 Hz), 7.77 (2H, d, J=8.3 Hz), 7.93 (2H, d, J=8.7 Hz).

### Working Example 210

### 4-[2-(3-Chloro-5-fluorobenzyl)-1-benzothiophen-7-yl]benzamide

7-Bromo-2-(3-chloro-5-fluorobenzyl)-1-benzothiophene obtained in Reference Example 191 and (4-carbamoylphenyl)boronic acid were used in the same manner as in Working Example 9 to obtain the titled compound. Yield: 65%; melting point: 202-203° C. (ethyl acetate-hexane).

¹H-NMR (CDCl₃) δ: 4.18 (2H, s), 5.63 (1H, br s), 6.10 (1H, br s), 6.89 (1H, d, J=9.0 Hz), 6.94-7.01 (1H, m), 7.07 (1H, s), 7.14 (1H, s), 7.34 (1H, d, J=7.5 Hz), 7.45 (1H, t, J=7.5 Hz), 7.72 (1H, d, J=7.5 Hz), 7.77 (2H, d, J=8.3 Hz), 7.93 (2H, d, J=8.7 Hz).

### Working Example 211

### 6-[2-[3-(Trifluoromethyl)benzyl]-1-benzothiophen-7-yl]pyridine-3-carboxamide

6-[2-[3-(Trifluoromethyl)benzyl]-1-benzothiophen-7-yl]pyridine-3-carboxylic acid obtained in Reference Example 242 was used in the same manner as in Working Example 197 to obtain the titled compound. Yield: 58%; melting point: 222-223° C. (THF-hexane).

¹H-NMR (DMSO-d₆) δ: 4.41 (2H, s), 7.32 (1H, s), 7.48-7.56 (1H, m), 7.63-7.70 (2H, m), 7.73 (1H, s), 7.92 (1H, d, J=8.0 Hz), 8.14 (1H, d, J=7.6 Hz), 8.20 (1H, br s), 8.27-8.41 (2H, m), 9.18 (1H, s), 2H unconfirmed.

### Working Example 212

### N-(2-Hydroxyethyl)-6-[2-[3-(trifluoromethyl)benzyl]-1-benzothiophen-7-yl]pyridine-3-carboxamide

6-[2-[3-(Trifluoromethyl)benzyl]-1-benzothiophen-7-yl]pyridine-3-carboxylic acid obtained in Reference Example 242 and 2-aminoethanol were used in the same manner as in Working Example 22 to obtain the titled compound. Yield: 26%; melting point: 160-161° C. (ethyl acetate-hexane).

¹H-NMR (CDCl₃) δ: 2.27 (1H, t, J=4.9 Hz), 3.69 (2H, q, J=5.4 Hz), 3.88 (2H, q, J=4.8 Hz), 4.32 (2H, s), 6.69 (1H, br s), 7.10 (1H, s), 7.39-7.54 (4H, m), 7.58 (1H, s), 7.80 (1H, d, J=7.5 Hz), 7.88 (1H, d, J=7.5 Hz), 8.02 (1H, d, J=8.7 Hz), 8.25 (1H, dd, J=8.3, 2.3 Hz), 9.15 (1H, d, J=1.5 Hz).

### Working Example 213

### N-(2-Amino-2-oxoethyl)-6-[2-[3-(trifluoromethyl)benzyl]-1-benzothiophen-7-yl]pyridine-3-carboxamide

6-[2-[3-(Trifluoromethyl)benzyl]-1-benzothiophen-7-yl]pyridine-3-carboxylic acid obtained in Reference Example 242 and glycinamide hydrochloride were used in the same manner as in Working Example 196 to obtain the titled compound. Yield: 80%; melting point: 191-192° C. (THF-hexane).

¹H-NMR (DMSO-d₆) δ: 3.86 (2H, d, J=5.7 Hz), 4.41 (2H, s), 7.08 (1H, br s), 7.32 (1H, s), 7.45 (1H, br s), 7.49-7.56 (1H, m), 7.57-7.65 (2H, m), 7.66-7.71 (1H, m), 7.74 (1H, s), 7.92 (1H, d, J=8.0 Hz), 8.14 (1H, d, J=7.6 Hz), 8.28-8.43 (2H, m), 8.98 (1H, t, J=5.7 Hz), 9.20 (1H, s).

### Working Example 214

### 5-[2-[3-(Trifluoromethyl)benzyl]-1-benzothiophen-7-yl]pyridine-2-carboxamide

4,4,5,5-Tetramethyl-2-[2-[3-(trifluoromethyl)benzyl]-1-benzothiophen-7-yl]-1,3,2-dioxaborolane obtained in Reference Example 193 and 5-bromopyridine-2-carboxamide were used in the same manner as in Working Example 9 to obtain the titled compound. Yield: 15%; melting point: 144-145° C. (ethyl acetate-hexane).

¹H-NMR (CDCl₃) δ: 4.29 (2H, s), 5.58 (1H, br s), 7.14 (1H, s), 7.34 (1H, d, J=7.2 Hz), 7.43-7.50 (3H, m), 7.50-7.58 (2H, m), 7.76 (1H, d, J=8.0 Hz), 7.86 (1H, br s), 8.16 (1H, dd, J=8.1, 2.1 Hz), 8.31 (1H, d, J=7.6 Hz), 8.87 (1H, d, J=1.9 Hz).

### Working Example 215

### N-(2-Cyanoethyl)-3-[2-(3,4-difluorobenzyl)-1-benzothiophen-4-yl]benzamide

3-[2-(3,4-Difluorobenzyl)-1-benzothiophen-4-yl]benzoic acid obtained in Reference Example 250 and 3-aminopropanenitrile were used in the same manner as in Working Example 3 to obtain the titled compound. Yield: 45%; melting point: 123-124° C. (ethyl acetate-hexane).

¹H-NMR (CDCl₃) δ: 2.79 (2H, t, J=6.3 Hz), 3.75 (2H, q, J=6.1 Hz), 4.17 (2 H, s), 6.62 (1H, br s), 6.94-7.01 (1H, m), 7.01-7.14 (3H, m), 7.29-7.40 (2H, m), 7.57 (1H, t, J=7.5 Hz), 7.72 (1H, ddd, J=7.8, 1.5, 1.4 Hz), 7.77 (1H, d, J=6.9 Hz), 7.81 (1H, dt, J=7.7, 1.5 Hz), 7.95 (1H, t, J=1.5 Hz)

### Working Example 216

### N-(2-Amino-2-oxoethyl)-3-[2-(3,4-difluorobenzyl)-1-benzothiophen-4-yl]benzamide

3-[2-(3,4-Difluorobenzyl)-1-benzothiophen-4-yl]benzoic acid obtained in Reference Example 250 and glycinamide hydrochloride were used in the same manner as in Working Example 12 to obtain the titled compound. Yield: 58%; melting point: 160-161° C. (ethyl acetate).

¹H-NMR (CDCl₃) δ: 4.17 (2H, s), 4.20 (2H, d, J=4.9 Hz), 5.49 (1H, br. s.), 6.00 (1H, br s), 6.93-7.02 (2H, m), 7.01-7.15 (3H, m), 7.29-7.40 (2H, m), 7.56 (1H, t, J=7.7 Hz), 7.70 (1H, ddd, J=8.0, 1.4, 1.1 Hz), 7.76 (1H, d, J=8.2 Hz), 7.84 (1H, dd, J=7.8, 1.8 Hz), 7.99 (1H, t, J=1.6 Hz)

### Working Example 217

### 3-[2-[3-(Trifluoromethyl)benzyl]-1-benzothiophen-7-yl]benzamide

28% Aqueous ammonia (500 μL, 7.3 mmol) was added to a DMF (3 mL) solution of ethyl 3-[2-[3-(trifluoromethyl)benzyl]-1-benzothiophen-7-yl]benzoate (300 mg, 0.73 mmol) obtained in Reference Example 200, WSC (167 mg, 0.87 mmol), and HOBt (118 mg, 0.87 mmol), and the mixture was stirred for 14 hours at room temperature. WSC (139 mg, 0.73 mmol), HOBt (98.0 mg, 0.73 mmol), and 28% aqueous ammonia (500 μL, 7.3 mmol) were furthermore added to the reaction solution, and the mixture was stirred for 5 hours at room temperature. The addition of water to the reaction solution was followed by extraction with ethyl acetate. The combined organic layers were washed with water and saturated brine, dried over anhydrous magnesium sulfate, and then concentrated at reduced pressure. The residue was purified by silica gel column chromatography (hexane-ethyl acetate 90:10→50:50) and was recrystallized from hexane-ethyl acetate to give 95.3 mg of the titled compound (yield 32%). Melting point: 153-154° C.

¹H-NMR (CDCl₃) δ: 4.28 (2H, s), 5.61 (1H, br s), 6.09 (1H, br s), 7.12 (1H, s), 7.29-7.36 (1H, m), 7.38-7.46 (3H, m), 7.47-7.55 (2H, m), 7.55-7.60 (1H, m), 7.70 (1H, dd, J=7.7, 1.1 Hz), 7.85 (2H, dd, J=7.7, 1.6 Hz), 8.09 (1H, t, J=1.5 Hz)

### Working Example 218

### N-Methyl-3-[2-[3-(trifluoromethyl)benzyl]-1-benzothiophen-7-yl]benzamide

A THF solution of ethyl 3-[2-[3-(trifluoromethyl)benzyl]-1-benzothiophen-7-yl]benzoate obtained in Reference Example 200 and methylamine was used in the same manner as in Working Example 3 to obtain the titled compound. Yield: 64%; melting point: 104-106° C. (ethyl acetate-hexane).

¹H-NMR (CDCl₃) δ: 3.04 (3H, d, J=4.9 Hz), 4.27 (2H, s), 6.14 (1H, br s), 7.11 (1H, s), 7.28-7.35 (1H, m), 7.38-7.58 (6H, m), 7.69 (1H, dd, J=8.0, 1.1 Hz), 7.75-7.85 (2H, m), 8.03 (1H, t, J=1.5 Hz)

### Working Example 219

### N-Ethyl-3-[2-[3-(trifluoromethyl)benzyl]-1-benzothiophen-7-yl]benzamide

A THF solution of ethyl 3-[2-[3-(trifluoromethyl)benzyl]-1-benzothiophen-7-yl]benzoate obtained in Reference Example 200 and ethylamine was used in the same manner as in Working Example 3 to obtain the titled compound. Yield: 54%; melting point: 122-123° C. (ethyl acetate-hexane).

¹H-NMR (CDCl₃) δ: 1.26 (3H, t, J=7.3 Hz), 3.52 (2H, qd, J=7.2, 5.8 Hz), 4.28 (2H, s), 6.08 (1H, br s), 7.11 (1H, s), 7.29-7.35 (1H, m), 7.38-7.47 (3H, m), 7.47-7.58 (3H, m), 7.69 (1H, dd, J=8.0, 1.1 Hz), 7.75-7.84 (2H, m), 8.02 (1H, t, J=1.6 Hz)

### Working Example 220

### 5-[2-(3-Chloro-5-fluorobenzyl)-1-benzothiophen-7-yl]-N-(2-hydroxyethyl)pyridine-3-carboxamide

A DMF (2.0 mL) solution of the 5-[2-(3-chloro-5-fluorobenzyl)-1-benzothiophen-7-yl]pyridine-3-carboxylic acid (100 mg, 0.251 mmol) obtained in Reference Example 251, 2-aminoethanol (0.016 mL, 0.276 mmol), WSC (57.8 mg, 0.302 mmol), and HOBt (40.8 mg, 0.302 mmol) was stirred for 3 hours at room temperature. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate) and recrystallized from hexane and ethyl acetate to give 69.5 mg of the titled compound (yield 63%) in solid form. Melting point: 126-127° C.

¹H NMR (DMSO-d₆) δ: 3.37 (2H, q, J=5.9 Hz), 3.54 (2H, q, J=5.9 Hz), 4.31 (2H, s), 4.76 (1H, t, J=5.9 Hz), 7.18-7.26 (1H, m), 7.26-7.34 (2H, m), 7.40 (1H, s), 7.44-7.58 (2H, m), 7.84-7.91 (1H, m), 8.47 (1H, t, J=2.1 Hz), 8.76 (1H, t, J=5.9 Hz), 8.99 (1H, d, J=1.9 Hz), 9.07 (1H, d, J=1.9 Hz).

### Working Example 221

### N-(2-Amino-2-oxoethyl)-5-[2-(3-chloro-5-fluorobenzyl)-1-benzothiophen-7-yl]pyridine-3-carboxamide

A DMF (2.0 mL) solution of the 5-[2-(3-chloro-5-fluorobenzyl)-1-benzothiophen-7-yl]pyridine-3-carboxylic acid (100 mg, 0.251 mmol) obtained in Reference Example 251, glycinamide hydrochloride (30.6 mg, 0.276 mmol), WSC (57.8 mg, 0.302 mmol), HOBt (40.8 mg, 0.302 mmol), and N,N-diisopropylethylamine (48.2 μL, 0.276 mmol) was stirred for 3 hours at room temperature. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate) and recrystallized from hexane and ethyl acetate to give 50.9 mg of the titled compound (yield 45%) in solid form. Melting point: 176-177° C.

¹H NMR (DMSO-d₆) δ: 3.86 (2H, d, J=5.7 Hz), 4.31 (2H, s), 7.09 (1H, br s), 7.22 (1H, d, J=9.4 Hz), 7.26-7.34 (2H, m), 7.36-7.59 (4H, m), 7.88 (1H, d, J=6.4 Hz), 8.49 (1H, t, J=2.1 Hz), 8.97-9.14 (3H, m).

### Working Example 222

### 6-[2-(3-Chloro-5-fluorobenzyl)-1-benzothiophen-7-yl]-N-(2-hydroxyethyl)pyridine-2-carboxamide

6-[2-(3-Chloro-5-fluorobenzyl)-1-benzothiophen-7-yl]pyridine-2-carboxylic acid obtained in Reference Example 252 and 2-aminoethanol were used in the same manner as in Working Example 220 to obtain the titled compound. Yield: 63%; melting point: 139-140° C. (ethyl acetate-hexane).

¹H NMR (DMSO-d₆) δ: 3.54 (2H, q, J=5.3 Hz), 3.67 (2H, q, J=5.3 Hz), 4.32 (2H, s), 5.05 (1H, t, J=5.3 Hz ), 7.25-7.34 (2H, m), 7.37 (2H, s), 7.55 (1H, t, J=7.8 Hz), 7.94 (1H, d, J=7.2 Hz), 8.03-8.10 (1H, m), 8.13-8.22 (2H, m), 8.46 (1H, d, J=7.2 Hz), 8.60 (1H, t, J=5.3 Hz).

### Working Example 223

### N-(2-Amino-2-oxoethyl)-6-[2-(3-chloro-5-fluorobenzyl)-1-benzothiophen-7-yl]pyridine-2-carboxamide

6-[2-(3-Chloro-5-fluorobenzyl)-1-benzothiophen-7-yl]pyridine-2-carboxylic acid obtained in Reference Example 252 and glycinamide hydrochloride were used in the same manner as in Working Example 221 to obtain the titled compound. Yield: 58%; melting point: 200-201° C. (ethyl acetate-hexane).

¹H NMR (DMSO-d₆) δ: 4.12 (2H, d, J=4.9 Hz), 4.35 (2H, s), 7.23-7.42 (5H, m), 7.55 (1H, t, J=7.8 Hz), 7.70 (1H, br s), 7.93 (1H, d, J=7.2 Hz), 8.03-8.10 (1H, m), 8.13-8.23 (2H, m), 8.48 (1H, d, J=7.2 Hz), 8.79 (1H, t, J=4.9 Hz).

### Working Example 224

### N-(2-Hydroxyethyl)-6-[2-[3-(trifluoromethyl)benzyl]-1-benzothiophen-7-yl]pyrimidine-4-carboxamide

A mixture of 4,4,5,5-tetramethyl-2-[2-[3-(trifluoromethyl)benzyl]-1-benzothiophen-7-yl]-1,3,2-dioxaborolane (430 mg, 1.03 mmol) obtained in Reference Example 193, ethyl 2,6-dichloropyrimidine-4-carboxylate (255 mg, 1.23 mmol), and tetrakis(triphenylphosphine)palladium (0) (143 mg, 0.123 mmol) in 2 N sodium carbonate aqueous solution (1.5 mL)-1,2-dimethoxyethane (8.6 mL) was stirred for 4 hours at 90° C. in a nitrogen atmosphere. Ethyl acetate and water were poured into the reaction solution, and the resulting solids were filtered off and dried to give 271 mg of 2-chloro-6-[2-[3-(trifluoromethyl)benzyl]-1-benzothiophen-7-yl]pyrimidine-4-carboxylic acid in solid form. A DMF (5.4 mL) solution of this compound (270 mg, 0.602 mmol), 2-aminoethanol (0.040 mL, 0.662 mmol), WSC (138 mg, 0.722 mmol), and HOBt (97.6 mg, 0.722 mmol) was stirred for 5 hours at room temperature. Water was poured into the reaction solution, and the mixture was extracted with ethyl acetate. The extract was washed with water and dried over anhydrous magnesium sulfate, and the solvent was distilled off at reduced pressure. The residue was purified by basic silica gel column chromatography (ethyl acetate:hexane=1:1) to give 77.2 mg of 2-chloro-N-(2-hydroxyethyl)-6-[2-[3-(trifluoromethyl)benzyl]-1-benzothiophen-7-yl]pyrimidine-4-carboxamide in solid form. Palladium-carbon (15 mg) was added in a nitrogen atmosphere to a THF (2 mL) solution of this compound (75 mg, 0.152 mmol) and triethylamine (0.064 mL, 0.457 mmol), and the reaction solution was stirred for 6 hours at room temperature in a 3 atm hydrogen atmosphere. The reaction solution was filtered, and the filtrate was concentrated. The residue was purified by silica gel column chromatography (ethyl acetate:hexane=4:1) and recrystallized from hexane and ethyl acetate to give 38.6 mg of the titled compound (yield 8%) in solid form. Melting point: 46-47° C.

¹H NMR (DMSO-d₆) δ: 3.43 (2H, q, J=6.2 Hz), 3.56 (2H, q, J=6.2 Hz), 4.44 (2H, s), 4.83 (1H, t, J=6.2 Hz ), 7.39 (1H, s), 7.52-7.72 (4H, m), 7.74 (1H, s), 8.05 (1H, d, J=7.2 Hz), 8.37 (1H, d, J=7.2 Hz), 8.71 (1H, s), 8.95 (1H, t, J=6.2 Hz), 9.47 (1H, s).

### Working Example 225

### 2-Fluoro-5-[2-[3-(trifluoromethyl)benzyl]-1-benzothiophen-7-yl]benzamide

2-Fluoro-5-[2-[3-(trifluoromethyl)benzyl]-1-benzothiophen-7-yl]benzoic acid obtained in Reference Example 253 and 28% aqueous ammonia were used in the same manner as in Working Example 197 to obtain the titled compound. Yield: 63%; melting point: 169-170° C. (ethyl acetate-hexane).

¹H NMR (DMSO-d₆) δ: 4.39 (2H, s), 7.33-7.41 (2H, m), 7.41-7.68 (5H, m), 7.71 (2H, s), 7.76-7.85 (3H, m), 7.90 (1H, dd, J=6.8, 2.7 Hz).

### Working Example 226

### 4-Fluoro-3-[2-[3-(trifluoromethyl)benzyl]-1-benzothiophen-7-yl]benzamide

4-Fluoro-3-[2-[3-(trifluoromethyl)benzyl]-1-benzothiophen-7-yl]benzoic acid obtained in Reference Example 254 and 28% aqueous ammonia were used in the same manner as in Working Example 197 to obtain the titled compound. Yield: 73%; melting point: 139-140° C. (ethyl acetate-hexane).

¹H NMR (DMSO-d₆) δ: 4.37 (2H, s), 7.31-7.39 (2H, m), 7.41-7.67 (6H, m), 7.71 (1H, s), 7.86 (1H, d, J=7.6 Hz), 7.97-8.14 (3H, m).

### Working Example 227

### N-(2-Amino-2-oxoethyl)-2-methyl-3-[2-[3-(trifluoromethyl)benzyl]-1-benzothiophen-7-yl]benzamide

2-Methyl-3-[2-[3-(trifluoromethyl)benzyl]-1-benzothiophen-7-yl]benzoic acid obtained in Reference Example 255 and glycinamide hydrochloride were used in the same manner as in Working Example 221 to obtain the titled compound. Yield: 61%; melting point: 124-125° C. (ethyl acetate-hexane).

¹H NMR (DMSO-d₆) δ: 2.05 (3H, s), 3.79 (2H, q, J=6.1 Hz ), 4.34 (2H, s), 7.04 (1H, br s), 7.12 (1H, d, J=6.8 Hz), 7.25-7.39 (4H, m), 7.40-7.50 (2H, m), 7.51-7.66 (3H, m), 7.70 (1H, s), 7.79 (1H, d, J=7.6 Hz), 8.49 (1H, d, J=6.1 Hz).

### Working Example 228

### 2-Fluoro-N-(2-methoxyethyl)-5-[2-[3-(trifluoromethyl)benzyl]-1-benzothiophen-7-yl]benzamide

2-Fluoro-5-[2-[3-(trifluoromethyl)benzyl]-1-benzothiophen-7-yl]benzoic acid obtained in Reference Example 253 and 2-methoxyethanamine were used in the same manner as in Working Example 220 to obtain the titled compound. Yield: 68%; melting point: 94-95° C. (diethyl ether-hexane).

¹H NMR (DMSO-d₆) δ: 3.26 (3H, s), 3.37-3.49 (4H, m), 4.39 (2H, s), 7.33-7.42 (2H, m), 7.42-7.67 (5H, m), 7.71 (1H, s), 7.76-7.87 (3H, m), 8.43 (1H, br s).

### Working Example 229

### 5-[2-(3-Chloro-5-fluorobenzyl)-1-benzothiophen-7-yl]-2-fluorobenzamide

5-[2-(3-Chloro-5-fluorobenzyl)-1-benzothiophen-7-yl]-2-fluorobenzoic acid obtained in Reference Example 256 and 28% aqueous ammonia were used in the same manner as in Working Example 197 to obtain the titled compound. Yield: 42%; melting point: 169-170° C. (ethyl acetate-hexane).

¹H NMR (DMSO-d₆) δ: 4.30 (2H, s), 7.22 (1H, d, J=9.8 Hz), 7.26-7.35 (2H, m), 7.35-7.52 (4H, m), 7.73 (1H, br s), 7.77-7.87 (3H, m), 7.90 (1H, dd, J=6.8, 2.6 Hz).

### Working Example 230

### N-(2-Amino-2-oxoethyl)-3-[4-fluoro-2-[3-(trifluoromethyl)benzyl]-1-benzothiophen-7-yl]benzamide

N-Ethyl diisopropylamine (57 μL, 0.30 mmol) was added to a DMF (25 mL) solution of 3-[4-fluoro-2-[3-(trifluoromethyl)benzyl]-1-benzothiophen-7-yl]benzoic acid (118 mg, 0.27 mmol) obtained in Reference Example 243, WSC (58 mg, 0.30 mmol), HOBt (41 mg, 0.30 mmol), and glycinamide hydrochloride (36 mg, 0.33 mmol), and the mixture was stirred for 5 hours at room temperature. The addition of saturated sodium bicarbonate aqueous solution to the reaction solution was followed by extraction with ethyl acetate. The combined organic layers were washed with water, dried over anhydrous sodium sulfate, and then concentrated at reduced pressure. The residue was purified by silica gel column chromatography (ethyl acetate-methanol 100:0→95:5) and was then recrystallized from a hexane-ethyl acetate solvent mixture to give 80 mg of the titled compound (yield 60%). Melting point: 157-158° C. (ethyl acetate-hexane).

¹H-NMR (CDCl₃) δ: 4.20 (2H, J=4.9 Hz), 4.27 (2H, s), 5.53 (1H, br s), 6.20 (1H, br s), 7.03-7.14 (2H, m), 7.22 (1H, s), 7.23-7.29 (1H, m), 7.39-7.58 (5H, m), 7.75-7.86 (2H, m), 8.04-8.09 (1H, m).

### Working Example 231

### 3-[4-Fluoro-2-[3-(trifluoromethyl)benzyl]-1-benzothiophen-7-yl]benzamide

3-[4-Fluoro-2-[3-(trifluoromethyl)benzyl]-1-benzothiophen-7-yl]benzoic acid obtained in Reference Example 243 and 28% aqueous ammonia were used in the same manner as in Working Example 197 to obtain the titled compound. Yield: 58%; melting point: 139-140° C. (ethyl acetate-hexane).

¹H-NMR (CDCl₃) δ: 4.28 (2H, s), 5.62 (1H, br s), 6.08 (1H, br s), 7.10 (1H, dd, J=9.6, 8.1 Hz), 7.21-7.29 (2H, m), 7.41-7.58 (5H, m), 7.75-7.85 (2H, m), 8.04 (1H, s).

### Working Example 232

### 3-[4-Fluoro-2-[3-(trifluoromethyl)benzyl]-1-benzothiophen-7-yl]-N-(2-methoxyethyl)benzamide

3-[4-Fluoro-2-[3-(trifluoromethyl)benzyl]-1-benzothiophen-7-yl]benzoic acid obtained in Reference Example 243 and 2-methoxyethanamine were used in the same manner as in Working Example 220 to obtain the titled compound. Yield: 64%; melting point: 123-124° C. (ethyl acetate-hexane).

¹H-NMR (CDCl₃) δ: 3.37 (3H, s), 3.56 (2H, t, J=5.1 Hz), 3.67 (2H, q, J=5.1 Hz), 4.27 (2H, s), 6.53 (1H, br s), 7.10 (1H, dd, J=9.6, 8.1 Hz), 7.21-7.29 (2H, m), 7.41-7.56 (5H, m), 7.73-7.80 (2H, m), 8.00 (1H, s).

### Working Example 233

### N-(2-Amino-2-oxoethyl)-3-[2-(3-chloro-5-fluorobenzyl)-4-fluoro-1-benzothiophen-7-yl]benzamide

3-[2-(3-Chloro-5-fluorobenzyl)-4-fluoro-1-benzothiophen-7-yl]benzoic acid obtained in Reference Example 244 and glycinamide hydrochloride were used in the same manner as in Working Example 230 to obtain the titled compound. Yield: 58%; melting point: 166-167° C. (ethyl acetate-hexane).

¹H-NMR (CDCl₃) δ: 4.16 (2H, s), 4.20 (2H, d, J=4.8 Hz), 5.62 (1H, br s), 6.37 (1H, br s), 6.87 (1H, d, J=8.4 Hz), 6.96 (1H, d, J=8.4 Hz), 7.04 (1H, s), 7.09 (1H, t, J=9.0 Hz), 7.18-7.24 (3H, m), 7.52 (1H, t, J=7.5 Hz), 7.77 (1H, d, J=8.4 Hz), 7.81 (1H, d, J=7.8 Hz), 8.05 (1H, s).

### Working Example 234

### 3-[2-(3-Chloro-5-fluorobenzyl)-4-fluoro-1-benzothiophen-7-yl]benzamide

3-[2-(3-Chloro-5-fluorobenzyl)-4-fluoro-1-benzothiophen-7-yl]benzoic acid obtained in Reference Example 244 and 28% aqueous ammonia were used in the same manner as in Working Example 197 to obtain the titled compound. Yield: 54%; melting point: 168-169° C. (ethyl acetate-hexane).

¹H-NMR(CDCl₃) δ: 4.18 (2H, s), 5.78 (1H, br s), 6.10 (1H, br s), 6.87 (1H, d, J=9.3 Hz), 6.97 (1H, d, J=8.4 Hz), 7.05 (1H, s), 7. 10 (1H, t, J=8.7 Hz), 7.20-7.30 (2H, m), 7.55 (1H, t, J=7.8 Hz), 7.77-7.85 (2H, m), 8.05 (1H, s).

### Working Example 235

### N-(2-Amino-2-oxoethyl)-3-[2-(3,4-difluorobenzyl)-4-fluoro-1-benzothiophen-7-yl]benzamide

3-[2-(3,4-Difluorobenzyl)-4-fluoro-1-benzothiophen-7-yl]benzoic acid obtained in Reference Example 245 and glycinamide hydrochloride were used in the same manner as in Working Example 230 to obtain the titled compound. Yield: 70%; melting point: 161-162° C. (ethyl acetate-hexane).

¹H-NMR (CDCl₃) δ: 4.14-4.21 (4H, m), 5.50 (1H, br s), 6.00 (1H, br s), 6.90-7.18 (5H, m), 7.20-7.27 (2H, m), 7.54 (1H, t, J=7.8 Hz), 7.78 (1H, d, J=7.5 Hz), 7.82 (1H, d, J=8.4 Hz), 8.05 (1H, s).

### Working Example 236

### N-(2-Amino-2-oxoethyl)-3-[4-chloro-2-(3,4-difluorobenzyl)-1-benzothiophen-7-yl]benzamide

3-[4-Chloro-2-(3,4-difluorobenzyl)-1-benzothiophen-7-yl]benzoic acid obtained in Reference Example 246 and glycinamide hydrochloride were used in the same manner as in Working Example 230 to obtain the titled compound. Yield: 59%; melting point: 188-189° C. (ethyl acetate-hexane).

¹H-NMR (CDCl₃) δ: 4.15-4.22 (4H, m), 5.50 (1H, br s), 6.12 (1H, br s), 6.92-7.12 (4H, m), 7.20-7.28 (2H, m), 7.41 (1H, d, J=7.8 Hz), 7.53 (1H, t, J=7.8 Hz), 7.75-7.86 (2H, m), 8.06 (1H, t, J=1.8 Hz).

### Working Example 237

### 3-[4-Chloro-2-(3,4-difluorobenzyl)-1-benzothiophen-7-yl]benzamide

3-[4-Chloro-2-(3,4-difluorobenzyl)-1-benzothiophen-7-yl]benzoic acid obtained in Reference Example 246 and 28% aqueous ammonia were used in the same manner as in Working Example 197 to obtain the titled compound. Yield: 55%; melting point: 185-186° C. (ethyl acetate-hexane).

¹H-NMR (CDCl₃ ) δ: 4.18 (2H, s), 5.71 (1H, br s), 6.08 (1H, br s), 6.90-7.15 (3H, m), 7.18-7.31 (2H, m), 7.42 (1H, d, J=8.1 Hz), 7.55 (1H, t, J=8.1 Hz), 7.74-7.85 (2H, m), 8.05 (1H, t, J=1.8 Hz).

### Working Example 238

### N-(2-Amino-2-oxoethyl)-3-[4-chloro-2-(3-chloro-5-fluorobenzyl)-1-benzothiophen-7-yl]benzamide

3-[4-Chloro-2-(3-chloro-5-fluorobenzyl)-1-benzothiophen-7-yl]benzoic acid obtained in Reference Example 247 and glycinamide hydrochloride were used in the same manner as in Working Example 230 to obtain the titled compound. Yield: 63%; melting point: 204-205° C. (ethyl acetate-hexane).

¹H-NMR (DMSO-d₆) δ: 3.83 (2H, d, J=5.7 Hz), 4.35 (2H, s), 7.05 (1H, br s), 7.21-7.45 (5H, m), 7.49 (1H, s), 7.57-7.66 (3H, m), 7.81 (1H, d, J=8.4 Hz), 7.96 (1H, t, J=7.8 Hz), 8.12 (1H, s), 8.82 (1H, t, J=6.0 Hz).

### Working Example 239

### 3-[4-Chloro-2-(3-chloro-5-fluorobenzyl)-1-benzothiophen-7-yl]benzamide

3-[4-Chloro-2-(3-chloro-5-fluorobenzyl)-1-benzothiophen-7-yl]benzoic acid obtained in Reference Example 247 and 28% aqueous ammonia were used in the same manner as in Working Example 197 to obtain the titled compound. Yield: 71%; melting point: 169-170° C. (ethyl acetate-hexane).

¹H-NMR (CDCl₃) δ: 4.19 (2H, s), 5.63 (1H, br s), 6.08 (1H, br s), 6.88 (1H, d, J=9.0 Hz), 6.97 (1H, d, J=9.0 Hz), 7.24 (1H, s), 7.25-7.29 (1H, m), 7.32 (1H, s), 7.43 (1H, d, J=7.8 Hz), 7.56 (1H, t, J=7.8 Hz), 7.77-7.85 (2H, m), 8.05 (1H, s).

### Working Example 240

### 3-Fluoro-5-[2-[3-(trifluoromethyl)benzyl]-1-benzothiophen-7-yl]benzamide

3-Fluoro-5-[2-[3-(trifluoromethyl)benzyl]-1-benzothiophen-7-yl]benzoic acid obtained in Reference Example 248 and 28% aqueous ammonia were used in the same manner as in Working Example 197 to obtain the titled compound. Yield: 55%; melting point: 165-166° C. (ethyl acetate-hexane).

¹H-NMR (CDCl₃) δ: 4.27 (2H, s), 5.60 (1H, br s), 6.00 (1H, br s), 7.11 (1H, s), 7.31 (1H, d, J=7.2 Hz), 7.40-7.60 (7H, m), 7.71 (1H, d, J=7.5 Hz), 7.85 (1H, s).

### Working Example 241

### 3-Fluoro-N-(2-methoxyethyl)-5-[2-[3-(trifluoromethyl)benzyl]-1-benzothiophen-7-yl]benzamide

3-Fluoro-5-[2-[3-(trifluoromethyl)benzyl]-1-benzothiophen-7-yl]benzoic acid obtained in Reference Example 248 and 2-methoxyethanamine were used in the same manner as in Working Example 220 to obtain the titled compound. Yield: 56%; melting point: 103-104° C. (ethyl acetate-hexane). ¹H-NMR (CDCl₃) δ: 3.36 (3H, s), 3.56 (2H, q, J=4.5 Hz), 3.66 (2H, q, J=4.5 Hz), 4.27 (2H, s), 6.49 (1H, br s), 7.10 (1H, s), 7.30 (1H, d, J=7.2 Hz), 7.38-7.54 (7H, m), 7.70 (1H, d, J=7.5 Hz), 7.81 (1H, s).

### Working Example 242

### 3-[2-(3-Chloro-5-fluorobenzyl)-1-benzothiophen-7-yl]-5-fluoro-N-(2-methoxyethyl)benzamide

3-[2-(3-Chloro-5-fluorobenzyl)-1-benzothiophen-7-yl]-5-fluorobenzoic acid obtained in Reference Example 249 and 2-methoxyethanamine were used in the same manner as in Working Example 220 to obtain the titled compound. Yield: 46%; melting point: 72-73° C. (ethyl acetate-hexane).

¹H-NMR (CDCl₃) δ: 3.37 (3H, s), 3.57 (2H, q, J=4.8 Hz), 3.67 (2H, q, J=4.8 Hz), 4.18 (2H, s), 6.50 (1H, br s), 6.88 (1H, d, J=9.6 Hz), 6.97 (1H, d, J=8.1 Hz), 7.06 (1H, s), 7.13 (1H, s), 7.32 (1H, d, J=6.9 Hz), 7.44 (1H, t, J=7.5 Hz), 7.48-7.55 (2H, m), 7.72 (1H, d, J=8.1 Hz), 7.81 (1H, s).

### Working Example 243

### N-(2-Amino-2-oxoethyl)-3-[2-(3-chloro-5-fluorobenzyl)-1-benzothiophen-7-yl]-5-fluorobenzamide

3-[2-(3-Chloro-5-fluorobenzyl)-1-benzothiophen-7-yl]-5-fluorobenzoic acid obtained in Reference Example 249 and glycinamide hydrochloride were used in the same manner as in Working Example 230 to obtain the titled compound. Yield: 44%. Amorphous solids.

¹H-NMR(CDCl₃) δ: 4.07-4.30 (4H, m), 5.55 (1H, br s), 6.09 (1H, br s), 6.88 (1H, d, J=8.4 Hz), 6.96 (1H, d, J=8.1 Hz), 7.00-7.18 (3H, m), 7.30 (1H, d, J=7.2 Hz), 7.42 (1H, t, J=7.8 Hz), 7.49-7.61 (2H, m), 7.71 (1H, d, J=8.1 Hz), 7.87 (1H, s).

### Working Example 244

### 3-[2-(3-Chloro-5-fluorobenzyl)-1-benzothiophen-7-yl]-5-fluorobenzamide

3-[2-(3-Chloro-5-fluorobenzyl)-1-benzothiophen-7-yl]-5-fluorobenzoic acid obtained in Reference Example 249 and 28% aqueous ammonia were used in the same manner as in Working Example 197 to obtain the titled compound. Yield: 56%; melting point: 186-187° C. (ethyl acetate-hexane).

¹H-NMR (CDCl₃) δ: 4.18 (2H, s), 5.70 (1H, br s), 6.00 (1H, br s), 6.88 (1H, d, J=9.9 Hz), 6.97 (1H, d, J=7.8 Hz), 7.06 (1H, s), 7.13 (1H, s), 7.31 (1H, d, J=7.5 Hz), 7.44 (1H, t, J=7.5 Hz), 7.52-7.58 (2H, m), 7.72 (1H, d, J=7.8 Hz), 7.85 (1H, s).

### Working Example 245

### 3-[2-(3-Chloro-5-fluorobenzyl)-1-benzothiophen-7-yl]-N-(2-cyanoethyl)-5-fluorobenzamide

3-[2-(3-Chloro-5-fluorobenzyl)-1-benzothiophen-7-yl]-5-fluorobenzoic acid obtained in Reference Example 249 and 3-aminopropanenitrile were used in the same manner as in Working Example 162 to obtain the titled compound. Yield: 56%; melting point: 113-114° C. (ethyl acetate-hexane).

¹H-NMR (CDCl₃) δ: 2.76 (2H, t, J=6.3 Hz), 3.73 (2H, q, J=6.3 Hz), 4.18 (2H, s), 6.59 (1H, br s), 6.88 (1H, d, J=8.7 Hz), 6.97 (1H, d, J=8.1 Hz), 7.06 (1H, s), 7.13 (1H, s), 7.31 (1H, d, J=7.5 Hz), 7.44 (1H, t, J=7.5 Hz), 7.50-7.57 (2H, m), 7.72 (1H, d, J=7.8 Hz), 7.83 (1H, s).

### Working Example 246

### N-(2-Methoxyethyl)-3-[2-[(2-methylpyridin-4-yl)methyl]-1-benzothiophen-7-yl]benzamide hydrochloride

A mixture of 3-[2-(bromomethyl)-1-benzothiophen-7-yl]-N-(2-methoxyethyl)benzamide (839 mg, 2.07 mmol) synthesized in Reference Example 187, 2-methyl-4-pyridineboronic acid pinacol ester (500 mg, 2.28 mmol), and tetrakis(triphenylphosphine)palladium (0) (120 mg, 0.104 mmol), 2 N sodium carbonate aqueous solution (3 mL), and 1,2-dimethoxyethane (6 mL) was stirred for 20 min at 150° C. while irradiated with microwaves. After cooling to room temperature, the reaction solution was partitioned with ethyl acetate and water. The organic phase was washed with saturated brine and dried over anhydrous magnesium sulfate, and the solvent was then distilled off at reduced pressure. The residue was purified by silica gel column chromatography (hexane-ethyl acetate=90:10→0/100). An ethyl acetate solution (1.0 mL) of 4 N hydrogen chloride was added to an ethyl acetate (4.0 mL) solution of the resulting pale yellow oily substance, the mixture was stirred for 5 min, and the solvent was then distilled off at reduced pressure to give 150 mg of the titled compound (yield 16%). Amorphous solids.

¹H-NMR (DMSO-d6) δ: 2.68 (3H, s), 3.27 (3H, s), 3.30-3.60 (4H, m), 4.56 (2H, s), 7.41-7.57 (3H, m), 7.61 (1H, t, J=7.8 Hz), 7.75-7.98 (5H, m), 8.14 (1H, s), 8.62-8.74 (2H, m).

### Working Example 247

### N-(2-Methoxyethyl)-3-(2-[[5-(trifluoromethyl)pyridin-3-yl]methyl]-1-benzothiophen-7-yl)benzamide hydrochloride

A DMF (15 mL) solution of 3-bromo-5-trifluoromethylpyridine (1.00 g, 4.42 mmol), bis(pinacolato)diboron (1.35 g, 5.30 mmol), [1,1-bis(diphenylphosphino)ferrocene]dichloropalladium (II) complex with dichloromethane (180 mg, 0.221 mmol), and potassium acetate (1.30 mg, 13.3 mmol) was stirred for 15 hours at 80° C. in an argon atmosphere. After the reaction solution had cooled to room temperature, 3-[2-(bromomethyl)-1-benzothiophen-7-yl]-N-(2-methoxyethyl)benzamide (1.49 g, 3.68 mmol) obtained in Reference Example 187, [1,1-bis(diphenylphosphino)ferrocene]dichloropalladium (II) complex with dichloromethane (90 mg, 0.111 mmol), and 2 N sodium carbonate aqueous solution (3.0 mL) were added to the reaction solution, and the mixture was stirred for 15 hours at 80° C. in an argon atmosphere. After cooling to room temperature, the reaction solution was partitioned with ethyl acetate and water. The organic phase was washed with saturated brine and dried over anhydrous magnesium sulfate, and the solvent was then distilled off at reduced pressure. The residue was purified by silica gel column chromatography (hexane-ethyl acetate=85:15→0/100). An ethyl acetate solution (1.0 mL) of 4 N hydrogen chloride was added to an ethyl acetate (4.0 mL) solution of the resulting pale yellow oily substance, the mixture was stirred for 5 min, and the solvent was then distilled off at reduced pressure to give 520 mg of the titled compound (yield 28%) in the form of pale yellow solids.

¹H-NMR (DMSO-d6) δ: 3.26 (3H, s), 3.37-3.53 (4H, m), 4.44 (2H, s), 7.36-7.45 (2H, m), 7.50 (1H, t, J=7.5 Hz), 7.61 (1H, t, J=7.5 Hz), 7.77-7.86 (2H, m), 7.88-7.95 (1H, m), 8.10-8.16 (1H, m), 8.18-8.25 (1H, m), 8.64 (1H, br s), 8.84-8.95 (2H, m).

### Working Example 248

### N-(2-Methoxyethyl)-3-(2-[[2-(trifluoromethyl)pyridin-4-yl]methyl]-1-benzothiophen-7-yl)benzamide

A DMF (5.0 mL) solution of 4-bromo-2-trifluoromethylpyridine (419 mg, 1.85 mmol), bis(pinacolato)diboron (564 mg, 2.22 mmol), [1,1-bis(diphenylphosphino)ferrocene]dichloropalladium (II) complex with dichloromethane (76 mg, 0.0925 mmol), and potassium acetate (545 mg, 5.55 mmol) was stirred for 15 hours at 80° C. in an argon atmosphere. After the reaction solution had cooled to room temperature, 3-[2-(bromomethyl)-1-benzothiophen-7-yl]-N-(2-methoxyethyl)benzamide (500 mg, 1.24 mmol) obtained in Reference Example 187, [1,1-bis(diphenylphosphino)ferrocene]dichloropalladium (II) complex with dichloromethane (38 mg, 0.0463 mmol), and 2 N sodium carbonate aqueous solution (1.0 mL) were added to the reaction solution, and the mixture was stirred for 15 hours at 80° C. in an argon atmosphere. After cooling to room temperature, the reaction solution was partitioned with ethyl acetate and water. The organic phase was washed with saturated brine and dried over anhydrous magnesium sulfate, and the solvent was then distilled off at reduced pressure. The residue was purified by silica gel column chromatography (hexane-ethyl acetate=85:15-0/100) to give 220 mg of the titled compound (yield 38%) in the form of a light tan oily substance.

¹H-NMR (CDCl₃) δ: 3.36 (3H, s), 3.50-3.60 (2H, m), 3.61-3.71 (2H, m), 4.29 (2H, s), 6.60 (1H, br s), 7.17 (1H, s), 7.34 (1H, dd, J=1.2, 7.2 Hz), 7.37-7.41 (1H, m), 7.45 (1H, t, J=7.5 Hz), 7.53 (1H, t, J=7.5 Hz), 7.57-7.61 (1H, m), 7.71 (1H, dd, J=1.2, 7.8 Hz), 7.79 (1H, d, J=1.8 Hz), 7.82 (1H, d, J=1.5 Hz), 8.06-8.10 (1H, m), 8.63 (1H, d, J=5.4 Hz).

### Working Example 249

### N-(2-Methoxyethyl)-3-[2-(pyridin-2-ylmethyl)-1-benzothiophen-7-yl]benzamide

A mixture of 3-[2-(bromomethyl)-1-benzothiophen-7-yl]-N-(2-methoxyethyl)benzamide (200 mg, 0.495 mmol) obtained in Reference Example 187, 2-(tributylstannyl)pyridine (218 mg, 0.593 mmol), and dichlorobis(triphenylphosphine)palladium (17 mg, 0.0248 mmol) in toluene (4.0 mL) was heated to reflux for 15 hours in an argon atmosphere. After cooling to room temperature, the reaction solution was partitioned with ethyl acetate and water. The organic phase was washed with saturated brine and dried over anhydrous magnesium sulfate, and the solvent was then distilled off at reduced pressure. The residue was purified by silica gel column chromatography (hexane-ethyl acetate=15:85-0/100) to give 70 mg of the titled compound (yield 35%) in the form of a colorless oily substance.

¹H-NMR (CDCl₃) δ: 3.37 (3H, s), 3.52-3.61 (2H, m), 3.62-3.72 (2H, m), 4.40 (2H, s), 6.56 (1H, br s), 7.11-7.18 (1H, m), 7.20 (1H, s), 7.22-7.27 (1H, m), 7.30 (1H, dd, J=1.2, 7.5 Hz), 7.41 (1H, dd, J=7.2, 8.1 Hz), 7.50-7.65 (2H, m), 7.69 (1H, d, J=1.2, 7.8 Hz), 7.78-7.86 (2H, m), 8.02-8.07 (1H, m), 8.54-8.59 (1H, m).

### Working Example 250

### N-(2-Methoxyethyl)-3-[2-[[4-(trifluoromethyl)pyridin-2-yl]methyl]-1-benzothiophen-7-yl]benzamide

A THF (5 mL) solution of 2-bromo-4-trifluoromethylpyridine (500 mg, 2.21 mmol) was cooled to −78° C., and a 1.6 N butyllithium-hexane solution (1.4 mL, 2.21 mmol) was added. The mixture was stirred at the same temperature for 15 min, tributyltin chloride (755 mg, 2.31 mmol) was then added, and the mixture was stirred for another hour at the same temperature. The addition of saturated ammonium chloride aqueous solution to the reaction solution was followed by extraction with ethyl acetate. The extract was washed with saturated brine and dried over anhydrous magnesium sulfate, and the solvent was then distilled off at reduced pressure. 3-[2-(Bromomethyl)-1-benzothiophen-7-yl]-N-(2-methoxyethyl)benzamide (404 mg, 1.00 mmol) obtained in Reference Example 187 and dichlorobis(triphenylphosphine)palladium (35 mg, 0.050 mmol) were added to a toluene (5 mL) solution of the residue, and the mixture was heated to reflux for 15 hours in an argon atmosphere. After cooling to room temperature, the reaction solution was partitioned with ethyl acetate and water. The organic phase was washed with saturated brine and dried over anhydrous magnesium sulfate, and the solvent was then distilled off at reduced pressure. The residue was purified by silica gel column chromatography (hexane-ethyl acetate=15:85-0/100) to give 110 mg of the titled compound (yield 23%) in the form of a colorless oily substance.

¹H-NMR (CDCl₃) δ: 3.37 (3H, s), 3.53-3.61 (2H, m), 3.62-3.72 (2H, m), 4.48 (2H, s), 6.57 (1H, br s), 7.23 (1H, s), 7.32 (1H, dd, J=1.2, 7.2 Hz), 7.36-7.48 (3H, m), 7.54 (1H, t, J=7.5 Hz), 7.71 (1H, dd, J=0.9, 7.5 Hz), 7.78-7.87 (2H, m), 8.02-8.08 (1H, m), 8.72-8.78 (1H, m).

### Working Example. 251

### N-(2-Methoxyethyl)-3-[2-[(5-methylpyridin-3-yl)methyl]-1-benzothiophen-7-yl]benzamide

A mixture of 3-[2-(bromomethyl)-1-benzothiophen-7-yl]-N-(2-methoxyethyl)benzamide (805 mg, 1.99 mmol) obtained in Reference Example 187, 5-methyl-3-pyridineboronic acid (300 mg, 2.19 mmol), tetrakis(triphenylphosphine)palladium (115 mg, 0.0995 mmol), 2 N sodium carbonate aqueous solution (3.0 mL), ethanol (2.0 mL), and 1,2-dimethoxyethane (6.0 mL) was stirred for 20 min at 150° C. while irradiated with microwaves. After cooling to room temperature, the reaction solution was partitioned with ethyl acetate and water. The organic phase was washed with saturated brine and dried over anhydrous magnesium sulfate, and the solvent was then distilled off at reduced pressure. The residue was purified by silica gel column chromatography (hexane-ethyl acetate=15:85-0/100) to give 150 mg of the titled compound (yield 18%) in the form of a colorless oily substance.

¹H-NMR (CDCl₃) δ: 2.30 (3H, s), 3.38 (3H, s), 3.52-3.61 (2H, m), 3.63-3.72 (2H, m), 4.18 (2H, s), 6.54 (1H, br s), 7.08-7.13 (1H, m), 7.32 (1H, dd, J=1.2, 7.5 Hz), 7.35-7.39 (1H, m), 7.43 (1H, t, J=7.8 Hz), 7.54 (1H, t, J=7.8 Hz), 7.69 (1H, dd, J=1.2, 7.8 Hz), 7.77-7.85 (2H, m), 8.03-8.07 (1H, m), 8.31-8.34 (1H, m), 8.36-8.40 (1H, m).

### Working Example 252

### N-(2-Amino-2-oxoethyl)-3-[2-[3-(trifluoromethyl)benzyl]-1,3-benzothiazol-4-yl]benzamide

3-[2-[3-(Trifluoromethyl)benzyl]-1,3-benzothiazol-4-yl]benzoic acid obtained in Reference Example 259 and glycinamide hydrochloride were used in the same manner as in Working Example 230 to obtain the titled compound. Yield: 43%; melting point: 130-131 ° C. (ethyl acetate-hexane).

¹H-NMR (CDCl₃) δ: 4.20 (2H, d, J=4.8 Hz), 4.50 (2H, s), 5.51 (1H, br s), 6.16 (1H, br s), 7.10 (1H, br s), 7.38-7.49 (2H, m), 7.53-7.59 (4H, m), 7.67 (1H, s), 7.78-7.87 (2H, m), 8.01 (1H, d, J=7.8 Hz), 8.31 (1H, t, J=1.8 Hz).

Table 3 shows the structures of the compounds obtained in Working Examples 142 through 252.

TABLE 3

Working Example 142

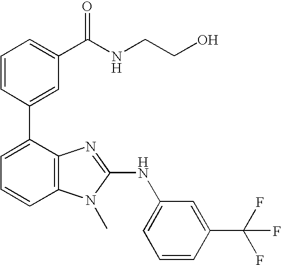

Working Example 143

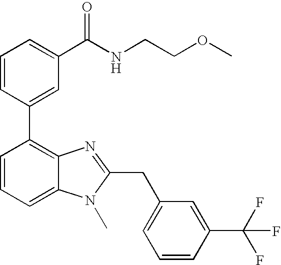

Working Example 144

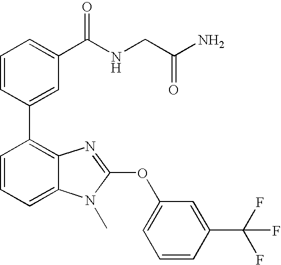

Working Example 145

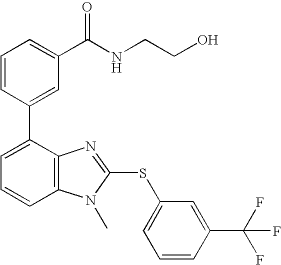

Working Example 146

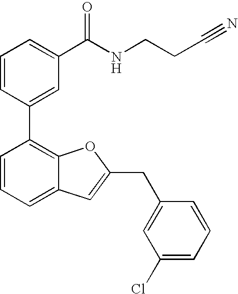

Working Example 147

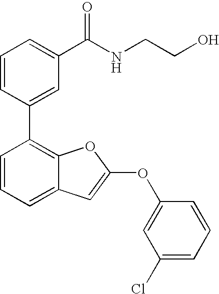

Working Example 148

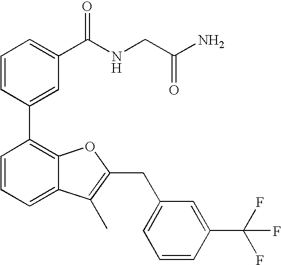

Working Example 149

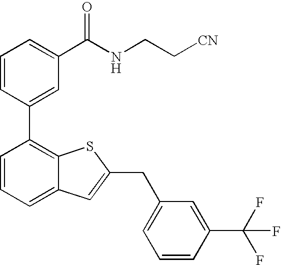

Working Example 150

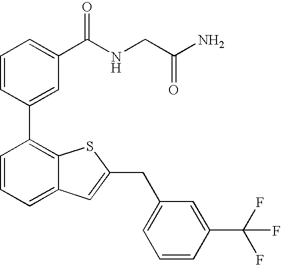

Working Example 151

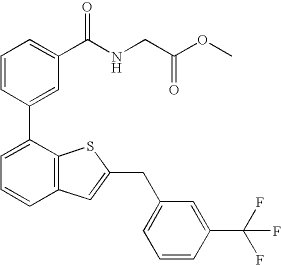

Working Example 152

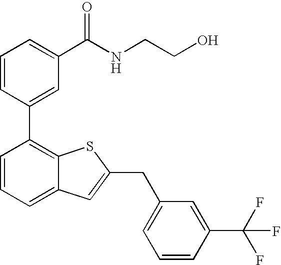

Working Example 153

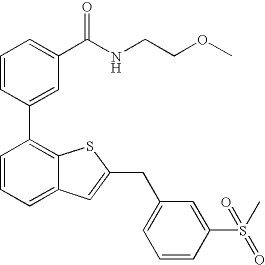

Working Example 154

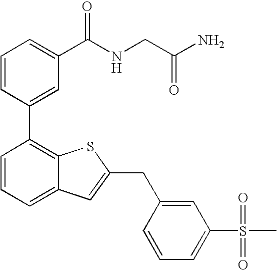

Working Example 155

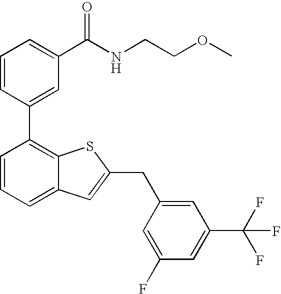

Working Example 156

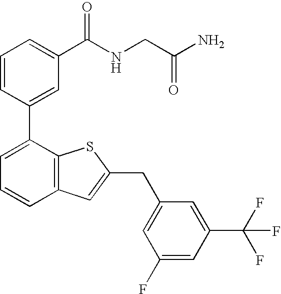

Working Example 157

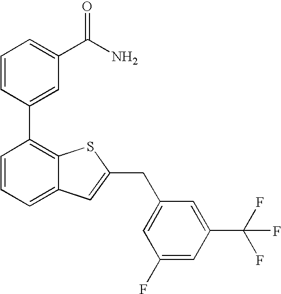

Working Example 158

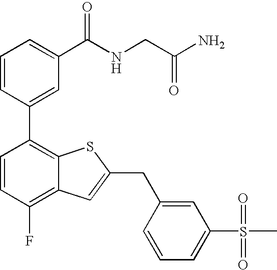

Working Example 159

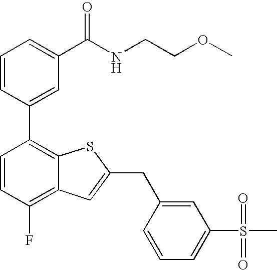

Working Example 160

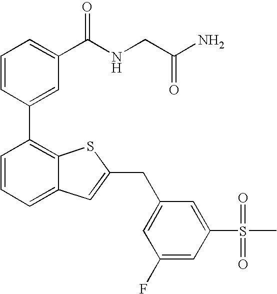

Working Example 161

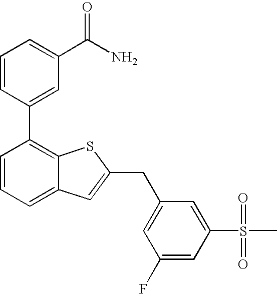

Working Example 162

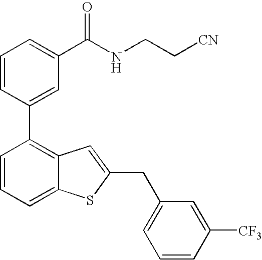

Working Example 163

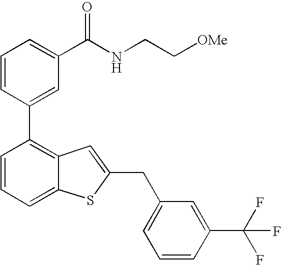

Working Example 164

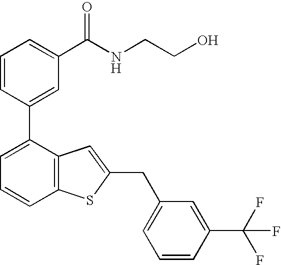

Working Example 165

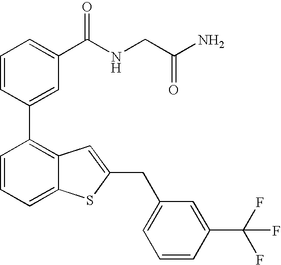

Working Example 166

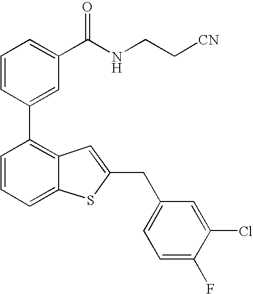

Working Example 167

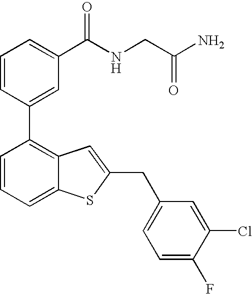

Working Example 168

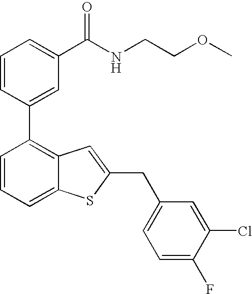

Working Example 169

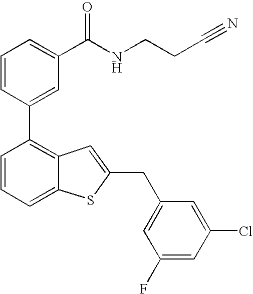

Working Example 170

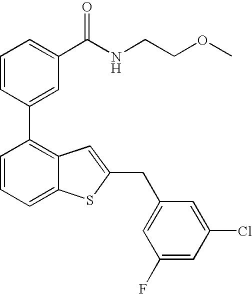

Working Example 171

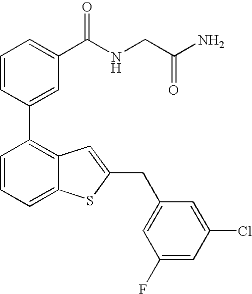

Working Example 172

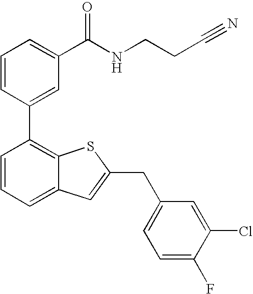

Working Example 173

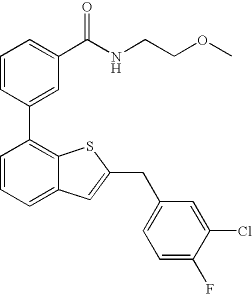

Working Example 174

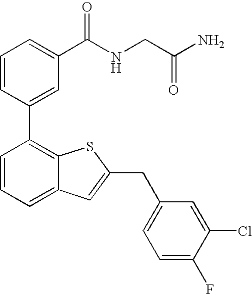

Working Example 175

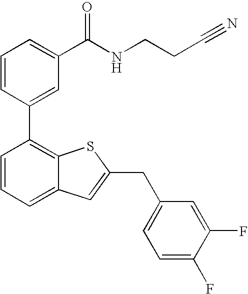

Working Example 176

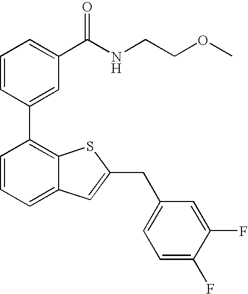

Working Example 177

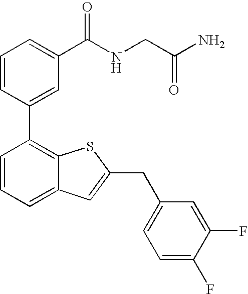

Working Example 178

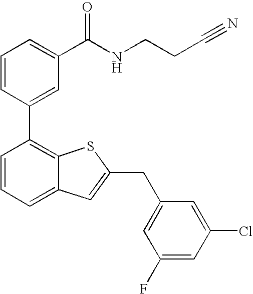

Working Example 179

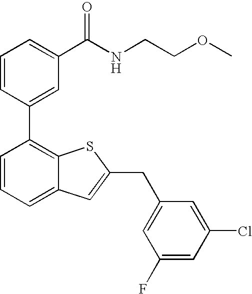

Working Example 180

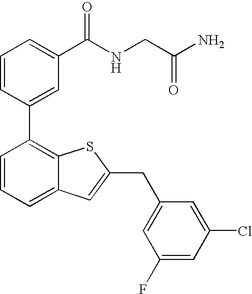

Working Example 181

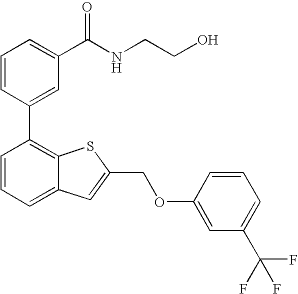

Working Example 182

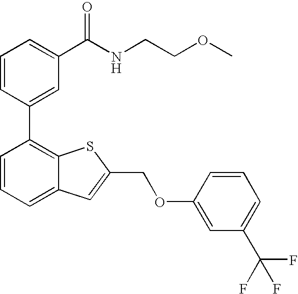

Working Example 183

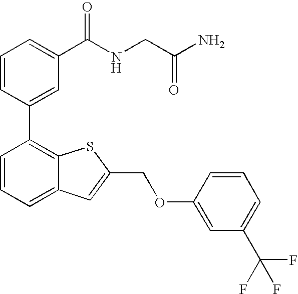

Working Example 184

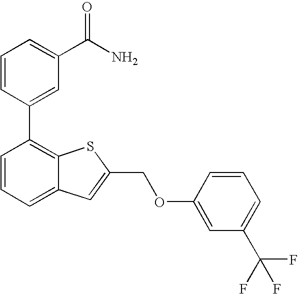

Working Example 185

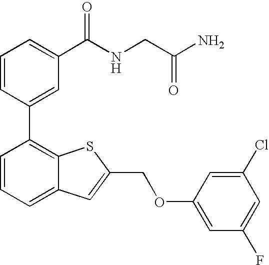

Working Example 186

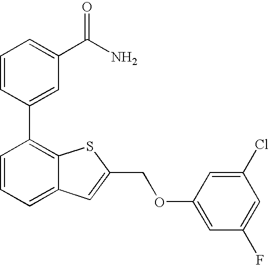

Working Example 187

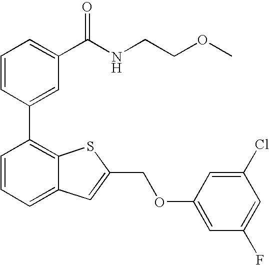

Working Example 188

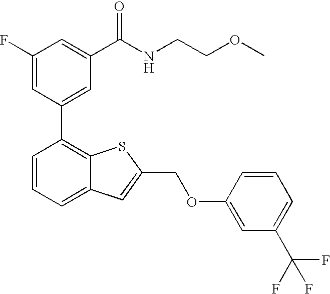

Working Example 189

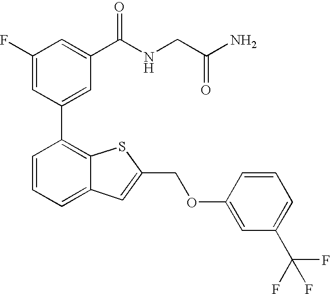

Working Example 190

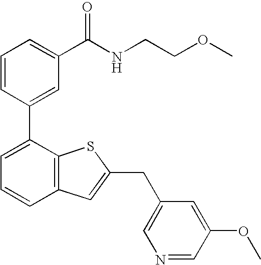

Working Example 191

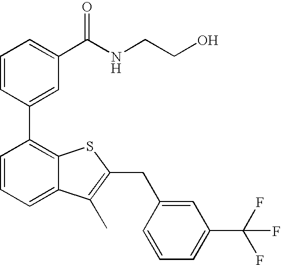

Working Example 192

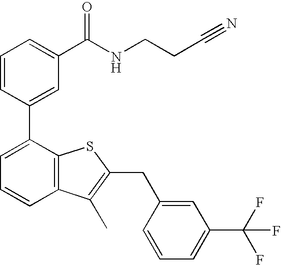

Working Example 193

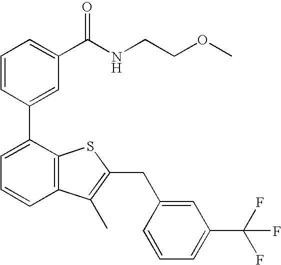

Working Example 194

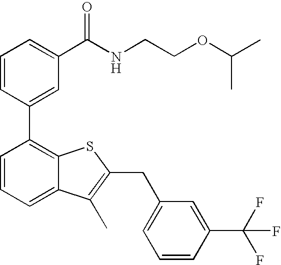

Working Example 195

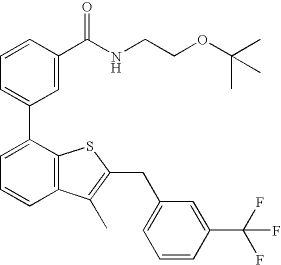

Working Example 196

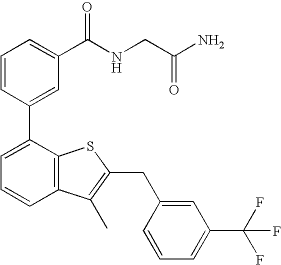

Working Example 197

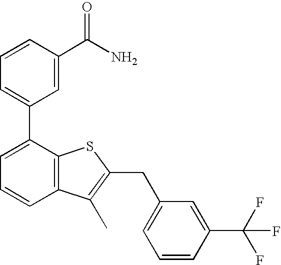

Working Example 198

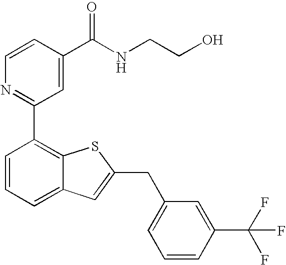

Working Example 199

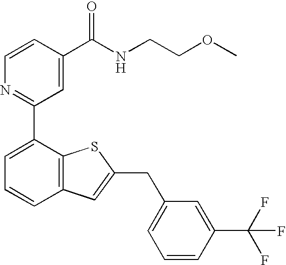

Working Example 200

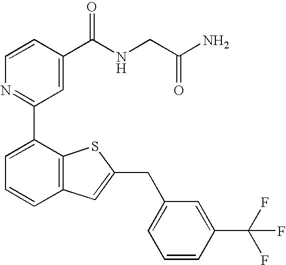

Working Example 201

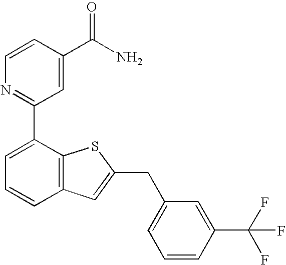

Working Example 202

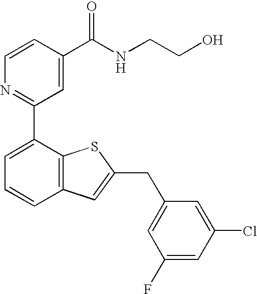

Working Example 203

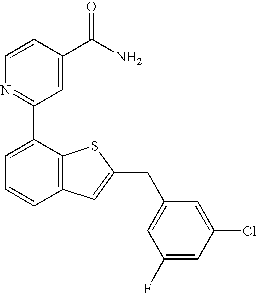

Working Example 204

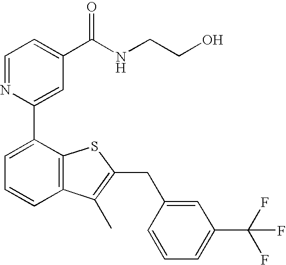

Working Example 205

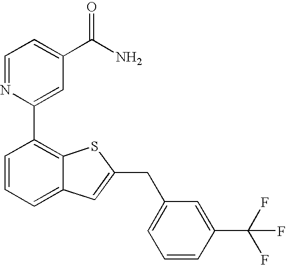

Working Example 206

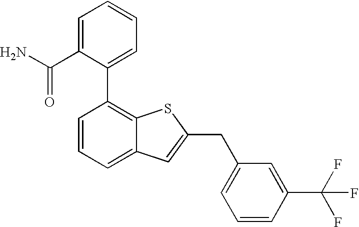

Working Example 207

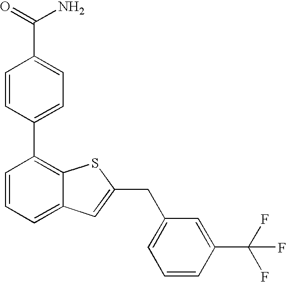

Working Example 208

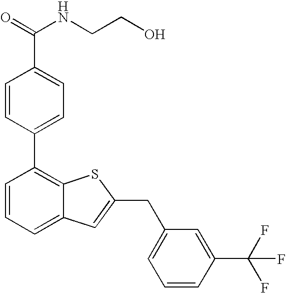

Working Example 209

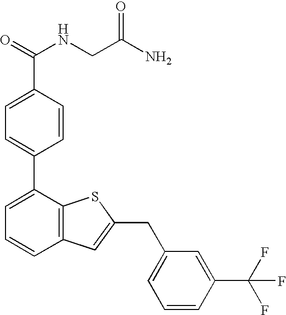

Working Example 210

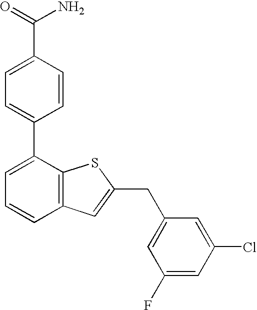

Working Example 211

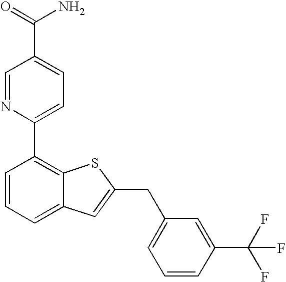

Working Example 212

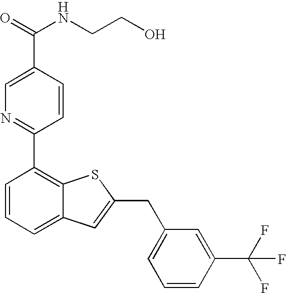

Working Example 213

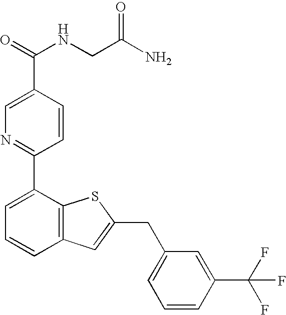

Working Example 214

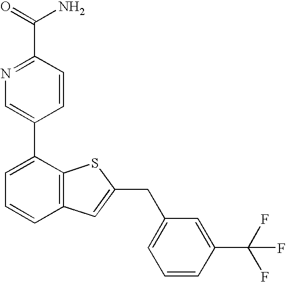

Working Example 215

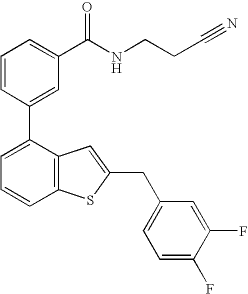

Working Example 216

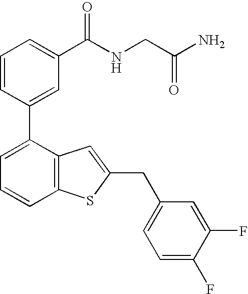

Working Example 217

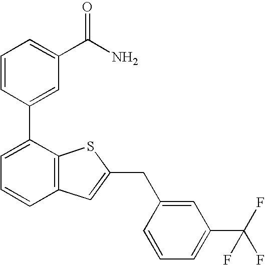

Working Example 218

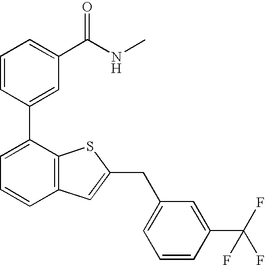

Working Example 219

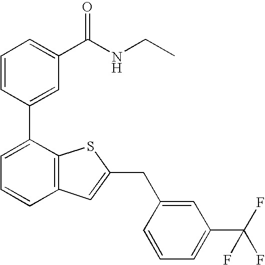

Working Example 220

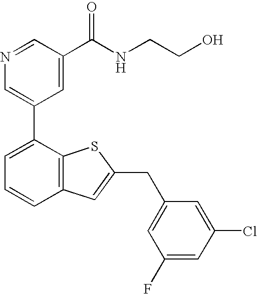

Working Example 221

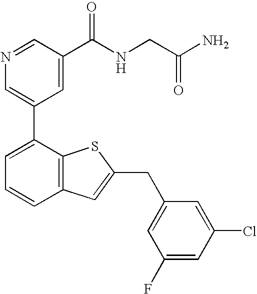

Working Example 222

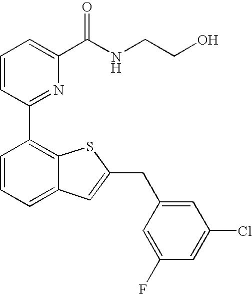

Working Example 223

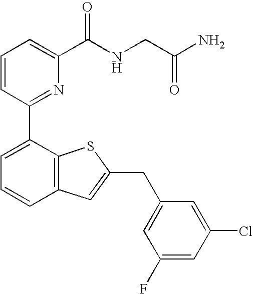

Working Example 224

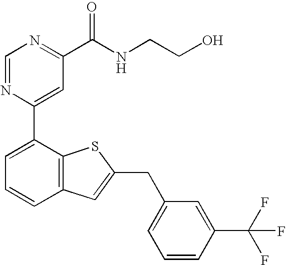

Working Example 225

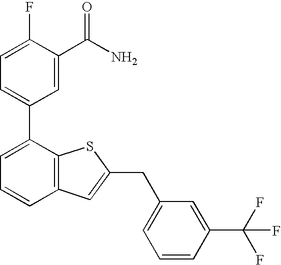

Working Example 226

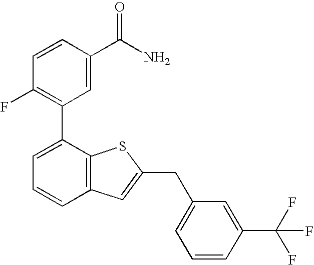

Working Example 227

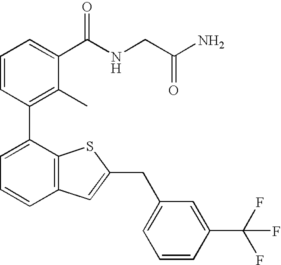

Working Example 228

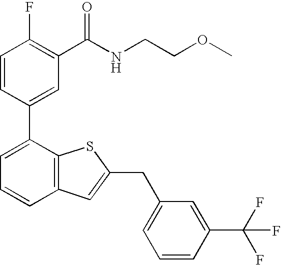

Working Example 229

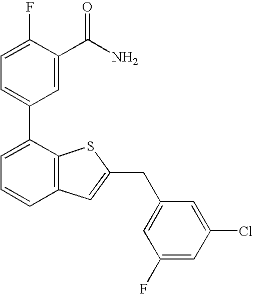

Working Example 230

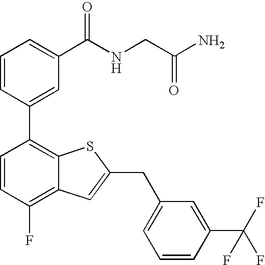

Working Example 231

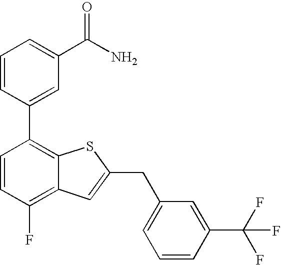

Working Example 232

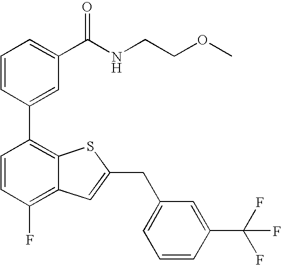

Working Example 233

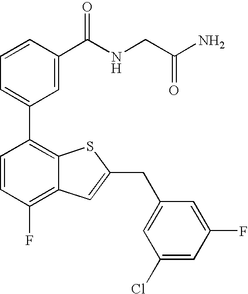

Working Example 234

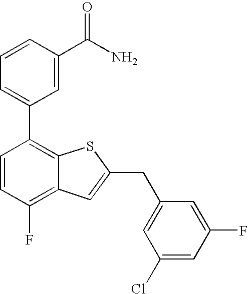

Working Example 235

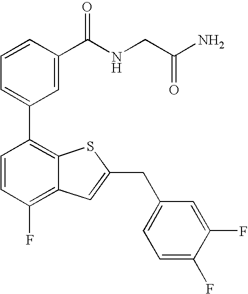

Working Example 236

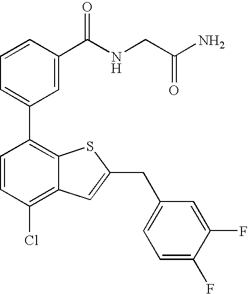

Working Example 237

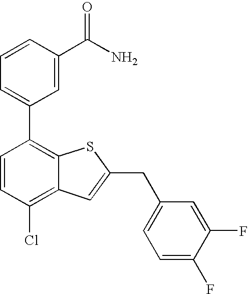

Working Example 238

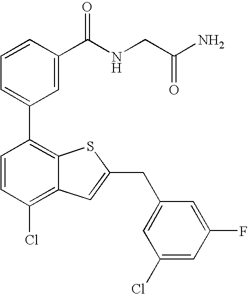

Working Example 239

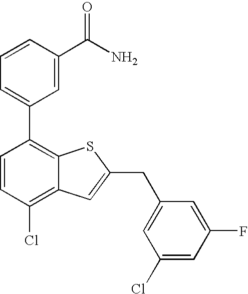

Working Example 240

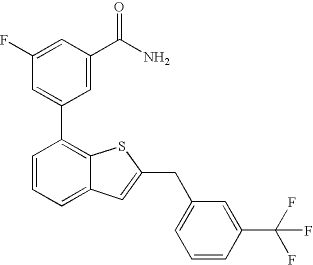

Working Example 241

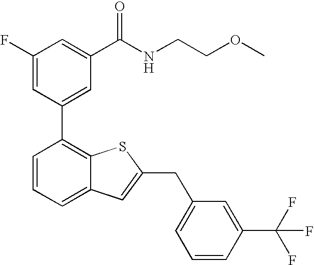

Working Example 242

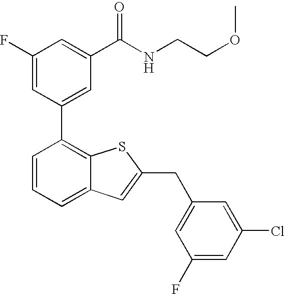

Working Example 243

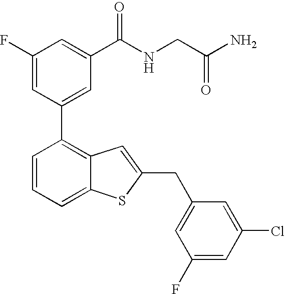

Working Example 244

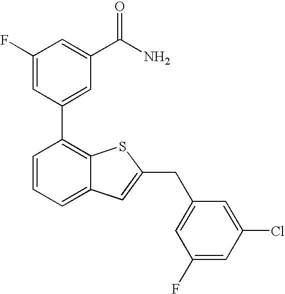

Working Example 245

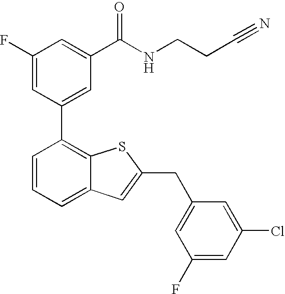

Working Example 246

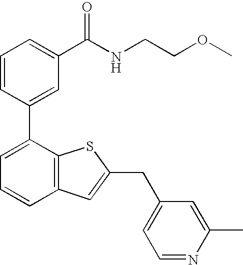

Working Example 247

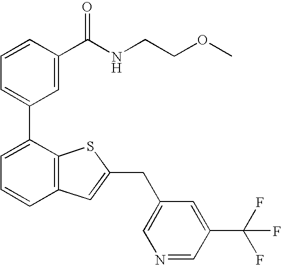

Working Example 248

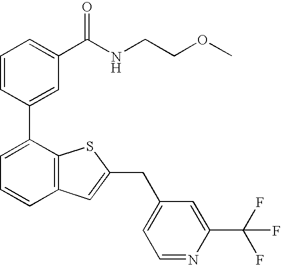

Working Example 249

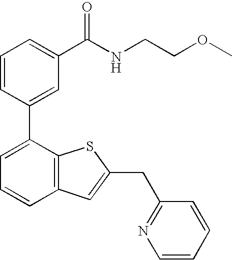

Working Example 250

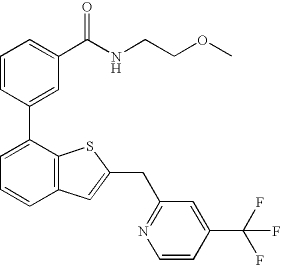

Working Example 251

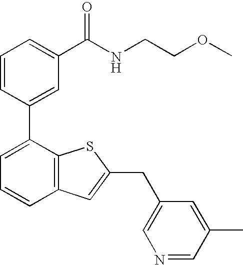

Working Example 252

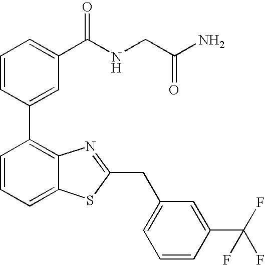

### Reference Example 260

### 7-Chlorothieno[3,2-b]pyridine-2-carbaldehyde

2.5 N n-butyllithium (9.63 mL, 24.1 mmol) was added at −50° C. to a THF solution (61.7 mL) of 7-chlorothieno[3,2-b]pyridine (3.14 g, 18.5 mmol), and the mixture was stirred for 1 hour. DMF (2.15 mL, 27.8 mmol) was added to the reaction solution at the same temperature, and the mixture was stirred for another 2 hours. Methanol was added to the reaction solution, the mixture was heated to room temperature, 1 N hydrochloric acid was added to adjust the pH to 1, and the mixture was then stirred for 30 min at room temperature. Saturated sodium bicarbonate aqueous solution was added to the mixture, and the resulting precipitate was filtered off, washed with water (80 mL) and diethyl ether (50 mL), and then dried to give 2.75 g of the titled product (yield 75%).

¹H-NMR (DMSO-d₆) δ: 7.82 (1H, d, J=5.2 Hz), 8.66 (1H, s), 8.83 (1H, d, J=5.2 Hz), 10.25 (1H, s).

### Reference Example 261

### (7-Chlorothieno[3,2-b]pyridin-2-yl)methanol

Sodium tetrahydroborate (632 mg, 16.7 mmol) was added while cooled on ice to an ethanol (6 mL) solution of 7-chlorothieno[3,2-b]pyridine-2-carbaldehyde (2.75 g, 13.9 mmol) obtained in Reference Example 260, and the mixture was then stirred for 1 hour at the same temperature. The addition of water to the reaction solution was followed by extraction with ethyl acetate. The organic layer was dried over anhydrous sodium sulfate, and then concentrated at reduced pressure to give 2.75 g of the titled compound (yield 99%) in the form of crystals.

¹H-NMR (DMSO-d₆) δ: 4.84 (2H, s), 5.98 (1H, br s), 7.48 (1H, dd, J=1.2, 1.2 Hz), 7.52 (1H, d, J=5.2 Hz), 8.60 (1H, d, J=5.6 Hz).

### Reference Example 262

### Ethyl 3-[2-(hydroxymethyl)thieno[3,2-b]pyridin-7-yl]benzoate

A water (8.0 mL)-THF (40 mL) mixture of (7-chlorothieno[3,2-b]pyridin-2-yl)methanol (2.75 g, 13.8 mmol) obtained in Reference Example 261, [3-(ethoxycarbonyl)phenyl]boronic acid (4.01 g, 20.7 mmol), dicyclohexyl[2′,4′,6′-trisisopropyl-biphenyl-2-yl]phosphine (657 mg, 1.38 mmol), palladium acetate (309 mg, 1.38 mmol), and tripotassium phosphate (8.77 g, 41.3 mmol) was stirred for 17 hours at 60° C. The reaction solution was diluted with dichloromethane, and was washed with water and saturated brine. The organic layer was dried over anhydrous sodium sulfate and then concentrated at reduced pressure, and the residue was purified by silica gel column chromatography (ethyl acetate) to give 2.71 g of the titled compound (yield 63%).

¹H-NMR (CDCl₃) δ: 1.43 (3H, t, J=7.2 Hz), 2.23 (1H, t, J=6.0 Hz), 4.43 (2H, q, J=7.2 Hz), 5.02 (2H, dd, J=6.0, 0.8 Hz), 7.32 (1H, d, J=4.8 Hz), 7.52 (1H, dd, J=0.8, 0.8 Hz), 7.63 (1H, td, J=7.6, 0.4 Hz), 7.95-7.96 (1H, m), 8.16-8.19 (1H, m), 8.41-8.42 (1H, m), 8.74 (1H, d, J=4.8 Hz).

### Reference Example 263

### Ethyl 3-[2-[3-(trifluoromethyl)benzyl]thieno[3,2-b]pyridin-7-yl]benzoate

Thionyl chloride (0.962 mL, 12.9 mmol) was added dropwise at 0° C. to a dichloromethane solution (30 mL) of ethyl 3-[2-(hydroxymethyl)thieno[3,2-b]pyridin-7-yl]benzoate (2.70 g, 8.62 mmol) obtained in Reference Example 262, and the mixture was stirred for 5 hours at room temperature. The solvent was distilled off at reduced pressure to give 2.86 g of ethyl 3-[2-(chloromethyl)thieno[3,2-b]pyridin-7-yl]benzoate crude product. A water (5 mL)-THF (25 mL) mixture of this compound (2.86 g, 8.62 mmol), [3-(trifluoromethyl)phenyl]boronic acid (2.13 g, 11.2 mmol), cesium carbonate (7.02 g, 21.6 mmol), and (1,1-bis(diphenylphosphino)ferrocene)dichloropalladium (II) complex with dichloromethane (352 mg, 0.431 mmol) was heated for 17 hours to 60° C. The reaction solution was diluted with water and extracted with dichloromethane. The organic layer was dried over anhydrous sodium sulfate and then concentrated at reduced pressure, and the residue was purified by silica gel column chromatography (hexane-ethyl acetate 67:33) to give 3.73 g of the titled compound (yield 98%).

¹H-NMR (CDCl₃) δ: 1.41 (3H, t, J=7.2 Hz), 4.34 (2H, s), 4.42 (2H, q, J=7.2 Hz), 7.29 (1H, d, J=4.8 Hz), 7.39 (1H, dd, J=1.2, 1.2 Hz), 7.45-7.56 (4H, m), 7.60 (1H, d, J=8.0, 0.8 Hz), 7.89-7.92 (1H, m), 8.14-8.16 (1H, m), 8.37-8.38 (1H, m), 8.73 (1H, d, J=6.0 Hz).

### Reference Example 264

### 3-[2-[3-(Trifluoromethyl)benzyl]thieno[3,2-b]pyridin-7-yl]benzoic acid

Ethyl 3-[2-[3-(trifluoromethyl)benzyl]thieno[3,2-b]pyridin-7-yl]benzoate obtained in Reference Example 263 was used in the same manner as in Reference Example 5 to obtain the titled compound. Yield: 94%.

¹H-NMR (DMSO-d₆) δ: 4.47 (2H, s), 7.43 (1H, d, J=4.8 Hz), 7.52 (1H, s), 7.55-7.63 (3H, m), 7.69 (1H, d, J=7.6 Hz), 7.77 (1H, s), 7.81 (1H, d, J=7.6 Hz), 8.03 (1H, dd, J=8.0, 1.2 Hz), 8.28 (1H, d, J=1.2 Hz), 8.69 (1H, d, J=5.2 Hz), 1H unconfirmed.

### Reference Example 265

### (7-Bromo-1-benzofuran-2-yl)(3-fluoro-4-methoxyphenyl)methanone

3-Bromo-2-hydroxybenzaldehyde, and 2-bromo-1-(3-fluoro-4-methoxyphenyl)ethanone were used in the same manner as in Reference Example 8 to obtain the titled compound. Yield: 32%.

¹H-NMR (CDCl₃) δ: 4.02 (3H, s), 7.11 (1H, t, J=8.4 Hz), 7.23 (2H, t, J=7.8 Hz), 7.68 (2H, td, J=8.6, 0.8 Hz), 8.02 (1H, dd, J=12.0, 2.0 Hz), 8.02 (1H, ddd, J=8.2, 1.2 Hz).

### Reference Example 266

### 7-Bromo-2-(3-fluoro-4-methoxybenzyl)-1-benzofuran

Triethylsilane (0.140 mL, 0.860 mmol) was added to a trifluoroacetic acid solution (2.0 mL) of (7-bromo-1-benzofuran-2-yl)(3-fluoro-4-methoxyphenyl)methanone (100 mg, 0.290 mmol) obtained in Reference Example 265, and the mixture was stirred for 5 hours at room temperature. The reaction solution was diluted with ethyl acetate, then washed with water, and then dried over anhydrous sodium sulfate. The solvent was distilled off at reduced pressure, and the resulting residue was purified by silica gel column chromatography (hexane-ethyl acetate 10:1) to give 64.2 mg of the titled compound (yield 67%).

¹H-NMR (CDCl₃) δ: 3.88 (3H, s), 4.07 (2H, s), 6.38 (1H, t, J=1.0 Hz), 6.91-6.95 (1H, m), 7.01-7.08 (3H, m), 7.38 (2H, td, J=7.8, 1.2 Hz).

### Reference Example 267

### Ethyl 3-[2-(3-fluoro-4-methoxybenzyl)-1-benzofuran-7-yl]benzoate

7-Bromo-2-(3-fluoro-4-methoxybenzyl)-1-benzofuran obtained in Reference Example 266, and [3-(ethoxycarbonyl)phenyl]boronic acid were used in the same manner as in Reference Example 4 to obtain the titled compound. Yield: 37%.

¹H-NMR (CDCl₃) δ: 1.41 (3H, t, J=7.2 Hz), 3.88 (3H, s), 4.06 (2H, s), 4.42 (2H, q, J=14.4, 7.2 Hz), 6.42 (1H, t, J=1.0 Hz), 6.92 (1H, t, J=8.6 Hz), 7.02-7.08 (2H, m), 7.28 (1H, t, J=7.4 Hz), 7.43 (1H, dd, J=7.6, 1.2 Hz), 7.47 (1H, dd, J=7.6, 1.6 Hz), 7.55 (1H, td, J=7.7, 0.4 Hz), 8.02-8.07 (2H, m), 8.51 (1H, t, J=2.0 Hz).

### Reference Example 268

### 3-[2-(3-Fluoro-4-methoxybenzyl)-1-benzofuran-7-yl]benzoic acid

Ethyl 3-[2-(3-fluoro-4-methoxybenzyl)-1-benzofuran-7-yl]benzoate obtained in Reference Example 267 was used in the same manner as in Reference Example 5 to obtain the titled compound. Yield: 94%.

¹H-NMR (CDCl₃) δ: 3.88 (3H, s), 4.06 (2H, s), 6.44 (1H, s), 6.94 (1H, t, J=8.4 Hz), 7.04-7.09 (2H, m), 7.25-7.33 (1H, m), 7.41-7.43 (2H, m), 7.59 (1H, br s), 8.07-8.12 (2H, m), 8.61 (1H, m), 1H unconfirmed.

### Reference Example 269

### (7-Bromo-1-benzofuran-2-yl)(3-chloro-4-fluorophenyl)methanone

3-Bromo-2-hydroxybenzaldehyde, and 2-bromo-1-(3-chloro-4-fluorophenyl)ethanone were used in the same manner as in Reference Example 8 to obtain the titled compound. Yield: 35%.

¹H-NMR (CDCl₃) δ: 7.25 (1H, t, J=7.8 Hz), 7.33 (1H, t, J=8.6 Hz), 7.68-7.72 (3H, m), 8.15-8.18 (1H, m), 8.33 (1H, dd, J=7.0, 2.2 Hz).

### Reference Example 270

### 7-Bromo-2-(3-chloro-4-fluorobenzyl)-1-benzofuran

(7-Bromo-1-benzofuran-2-yl)(3-chloro-4-fluorophenyl)methanone obtained in Reference Example 269 was used in the same manner as in Reference Example 266 to obtain the titled compound. Yield: 52%.

¹H-NMR (CDCl₃) δ: 4.12 (2H, s), 6.42 (1H, t, J=1.0 Hz), 7.04-7.13 (2H, m), 7.17-7.21 (1H, m), 7.34-7.38 (1H, m), 7.40 (2H, td, J=8.1, 0.8 Hz).

### Reference Example 271

### Ethyl 3-[2-(3-chloro-4-fluorobenzyl)-1-benzofuran-7-yl]benzoate

7-Bromo-2-(3-chloro-4-fluorobenzyl)-1-benzofuran obtained in Reference Example 270, and [3-(ethoxycarbonyl)phenyl]boronic acid were used in the same manner as in Reference Example 4 to obtain the titled compound. Yield: 33%.

¹H-NMR (CDCl₃) δ: 1.40 (3H, t, J=8.2 Hz), 4.08 (2H, s), 4.42 (2H, q, J=14.2, 7.0 Hz), 6.45 (1H, t, J=0.8 Hz), 7.10 (1H, t, J=8.8 Hz), 7.18-7.22 (1H, m), 7.29 (1H, t, J=7.6 Hz), 7.36 (1H, dd, J=7.0, 2.2 Hz), 7.43 (1H, dd, J=7.4, 1.0 Hz), 7.49 (1H, dd, J=7.6, 1.2 Hz), 7.55 (1H, td, J=7.8, 1.6 Hz), 7.99-8.02 (1H, m), 8.05-8.07 (1H, m), 8.51 (1H, t, J=1.6 Hz).

### Reference Example 272

### 3-[2-(3-Chloro-4-fluorobenzyl)-1-benzofuran-7-yl]benzoic acid

Ethyl 3-[2-(3-chloro-4-fluorobenzyl)-1-benzofuran-7-yl]benzoate obtained in Reference Example 271 was used in the same manner as in Reference Example 5 to obtain the titled compound. Yield: 97%.

¹H-NMR (CDCl₃) δ: 4.21 (2H, s), 6.64 (1H, s), 7.30 (1H, t, J=7.6 Hz), 7.39-7.44 (4H, m), 7.53 (1H, dd, J=7.6, 1.2 Hz), 7.60 (1H, d, J=8.0 Hz), 7.73-7.76 (1H, m), 7.55 (1H, dt, J=7.6, 1.6 Hz), 8.33 (1H, t, J=1.6 Hz), 1H unconfirmed.

### Reference Example 273

### (7-Bromo-1-benzofuran-2-yl)(3,4-difluorophenyl)methanone

3-Bromo-2-hydroxybenzaldehyde, and 2-bromo-1-(3,4-difluorophenyl)ethanone were used in the same manner as in Reference Example 8 to obtain the titled compound. Yield: 30%.

¹H-NMR (CDCl₃) δ: 7.12 (1H, t, J=7.2 Hz), 7.33-7.39 (1H, m), 7.68-7.71 (3H, m), 8.04-8.12 (2H, m).

### Reference Example 274

### 7-Bromo-2-(3,4-difluorobenzyl)-1-benzofuran

(7-Bromo-1-benzofuran-2-yl)(3,4-difluorophenyl)methanone obtained in Reference Example 273 was used in the same manner as in Reference Example 266 to obtain the titled compound. Yield: 42%.

¹H-NMR (CDCl₃) δ: 4.11 (2H, s), 6.42 (1H, d, J=0.8 Hz), 7.01-7.16 (4H, m), 7.40 (2H, dt, J=8.2, 1.2 Hz).

### Reference Example 275

### Ethyl 3-[2-(3,4-difluorobenzyl)-1-benzofuran-7-yl]benzoate

7-Bromo-2-(3,4-difluorobenzyl)-1-benzofuran obtained in Reference Example 274, and [3-(ethoxycarbonyl)phenyl]boronic acid were used in the same manner as in Reference Example 4 to obtain the titled compound. Yield: 61%

¹H-NMR (CDCl₃ ) δ: 1.40 (3H, t, J=7.6 Hz), 4.09 (2H, s), 4.42 (2H, q, J=14.2, 7.0 Hz), 6.45 (1H, t, J=1.0 Hz), 7.03-7.07 (1H, m), 7.09-7.16 (2H, m), 7.29 (1H, t, J=7.6 Hz), 7.36 (1H, dd, J=7.6, 1.2 Hz), 7.49 (1H, dd, J=7.6, 1.2 Hz), 7.55 (1H, t, J=7.8 Hz), 7.99-8.02 (1H, m), 8.05-8.07 (1H, m), 8.51 (1H, t, J=1.8 Hz).

### Reference Example 276

### 3-[2-(3,4-Difluorobenzyl)-1-benzofuran-7-yl]benzoic acid

Ethyl 3-[2-(3,4-difluorobenzyl)-1-benzofuran-7-yl]benzoate obtained in Reference Example 275 was used in the same manner as in Reference Example 5 to obtain the titled compound. Yield: 92%.

¹H-NMR (CDCl₃) δ: 4.19 (2H, s), 6.62 (1H, s), 7.24-7.29 (2H, m), 7.31-7.47 (4H, m), 7.52 (1H, dd, J=7.6, 1.2 Hz), 7.71-7.73 (1H, m), 7.86-7.88 (1H, m), 8.31 (1H, t, J=1.6 Hz), 1H unconfirmed.

### Reference Example 277

### 7-Chloro-4-fluoro-2-[3-(trifluoromethyl)benzyl]-1-benzothiophene

2-(Bromomethyl)-7-chloro-4-fluoro-1-benzothiophene obtained in Reference Example 184, and [3-(trifluoromethyl)phenyl]boronic acid were used in the same manner as in Reference Example 194 to obtain the titled compound. Yield: 75%

¹H-NMR (CDCl₃) δ: 4.29 (2H, s), 6.96 (1H, dd, J=9.6, 8.7 Hz), 7.14-7.27 (2H, m), 7.40-7.61 (4H, m).

### Reference Example 278

### Ethyl 3-fluoro-5-[4-fluoro-2-[3-(trifluoromethyl)benzyl]-1-benzothiophen-7-yl]benzoate

7-Chloro-4-fluoro-2-[3-(trifluoromethyl)benzyl]-1-benzothiophene obtained in Reference Example 277, and ethyl 3-fluoro-5-(4,4,5,5-tetramethyl-1,3,2-dioxaborolane-2-yl)benzoate obtained in Reference Example 225 was used in the same manner as in Reference Example 203 to obtain the titled compound. Yield: 68%

¹H-NMR (CDCl₃) δ: 4.29 (2H, s), 6.96 (1H, dd, J=9.6, 8.7 Hz), 7.14-7.27 (2H, m), 7.40-7.61 (4H, m).

### Reference Example 279

### 3-Fluoro-5-[4-fluoro-2-[3-(trifluoromethyl)benzyl]-1-benzothiophen-7-yl]benzoic acid

Ethyl 3-fluoro-5-[4-fluoro-2-[3-(trifluoromethyl)benzyl]-1-benzothiophen-7-yl]benzoate obtained in Reference Example 278 was used in the same manner as in Reference Example 5 to obtain the titled compound. Yield: 63% melting point: 169-170 ° C. (ethyl acetate-hexane).

¹H-NMR (CDCl₃) δ: 4.29 (2H, s), 7.11 (1H, dd, J=9.6, 8.1 Hz), 7.21-7.31 (3H, m), 7.41-7.54 (3H, m), 7.59 (1H, d, J=8.1 Hz), 7.80 (1H, d, J=7.5 Hz), 8.17 (1H, s), 1H unconfirmed.

### Reference Example 280

### Ethyl 4-fluoro-3-(4,4,5,5-tetramethyl-1,3,2-dioxaborolan-2-yl)benzoate

Ethyl 3-bromo-4-fluorobenzoate was used in the same manner as in Reference Example 225 to obtain the titled compound. Yield: 42%

¹H-NMR (CDCl₃) δ: 1.37 (12H, s), 1.42 (3H, t, J=7.2 Hz), 4.37 (2H, q, J=7.2 Hz), 7.07 (1H, t, J=8.7 Hz), 8.07-8.15 (1H, m), 8.37-8.44 (1H, m).

### Reference Example 281

### Ethyl 3-[2-(bromomethyl)-1-benzothiophen-7-yl]-4-fluorobenzoate

Ethyl 4-fluoro-3-(4,4,5,5-tetramethyl-1,3,2-dioxaborolan-2-yl)benzoate obtained in Reference Example 280, and (7-bromo-1-benzothiophene-2-yl)methanol synthesized in Reference Example 162 were used in the same manner as in Reference Examples 170, and 185 to obtain the titled compound. Yield: 58%, melting point: 143-144 ° C. (ethyl acetate-hexane).

¹H-NMR (CDCl₃) δ: 1.39 (3H, t, J=7.2 Hz), 4.39 (2H, q, J=7.2 Hz), 4.74 (2H, s), 7.11 (1H, dd, J=9.6, 8.1 Hz), 7.22-7.37 (3H, m), 7.49 (1H, s), 8.06-8.14 (1H, m), 8.25 (1H, dd, J=7.2, 2.1 Hz).

### Reference Example 282

### Ethyl 3-[2-(3-chloro-5-fluorobenzyl)-4-fluoro-1-benzothiophen-7-yl]-4-fluorobenzoate

Ethyl 3-[2-(bromomethyl)-1-benzothiophen-7-yl]-4-fluorobenzoate obtained in Reference Example 281 was used in the same manner as in Reference Example 197 to obtain the titled compound. Yield: 71%.

¹H-NMR (CDCl₃) δ: 1.38 (3H, t, J=7.2 Hz), 4.17 (2H, s), 4.37 (2H, q, J=7.2 Hz), 6.87 (1H, d, J=9.0 Hz), 6.96 (1H, d, J=8.4 Hz), 7.05 (1H, s), 7.10 (1H, dd, J=9.0, 8.7 Hz), 7.21-7.37 (3H, m), 8.05-8.12 (1H, m), 8.23 (1H, dd, J=7.2, 2.1 Hz).

### Reference Example 283

### 3-[2-(3-Chloro-5-fluorobenzyl)-4-fluoro-1-benzothiophen-7-yl]-4-fluorobenzoic acid

Ethyl 3-[2-(3-chloro-5-fluorobenzyl)-4-fluoro-1-benzothiophen-7-yl]-4-fluorobenzoate obtained in Reference Example 282 was used in the same manner as in Reference Example 5 to obtain the titled compound. Yield: 66%.

¹H-NMR (CDCl₃) δ: 4.17 (2H, s), 6.87 (1H, d, J=8.4 Hz), 6.96 (1H, d, J=8.7 Hz), 7.05 (1H, s), 7.10 (1H, t, J=8.7 Hz), 7.21-7.40 (3H, m), 8.10-8.20 (1H, m), 8.31 (1H, d, J=6.9 Hz), 1H unconfirmed.

### Reference Example 284

### (4-Bromo-1-benzofuran-2-yl)(3-chloro-4-fluorophenyl)methanone

2-Bromo-6-hydroxybenzaldehyde and 2-bromo-1-(3-chloro-4-fluorophenyl)ethanone were used in the same manner as in Reference Example 8 to obtain the titled compound. Yield: 63%.

¹H-NMR (CDCl₃) δ: 7.31-7.41 (2H, m), 7.51 (1H, dd, J=7.8, 0.6 Hz), 7.55-7.62 (2H, m), 8.02 (1H, ddd, J=8.4, 4.2, 1.8 Hz), 8.18 (1H, dd, J=7.2, 1.8 Hz).

### Reference Example 285

### 4-Bromo-2-(3-chloro-4-fluorobenzyl)-1-benzofuran

(4-Bromo-1-benzofuran-2-yl)(3-chloro-4-fluorophenyl)methanone obtained in Reference Example 284 was used in the same manner as in Reference Example 266 to obtain the titled compound. Yield: 42%.

¹H-NMR (CDCl₃) δ: 4.05 (2H, s), 6.45 (1H, s), 7.04-7.19 (3H, m), 7.30-7.37 (3H, m).

### Reference Example 286

### 3-[2-(3-Chloro-4-fluorobenzyl)-1-benzofuran-4-yl]benzoic acid

4-Bromo-2-(3-chloro-4-fluorobenzyl)-1-benzofuran obtained in Reference Example 285, and [3-(ethoxycarbonyl)phenyl]boronic acid were used in the same manner as in Reference Example 10 to obtain the titled compound. Yield: 64%.

¹H-NMR (CDCl₃) δ: 4.08 (2H, s), 6.61 (1H, s), 7.08 (1H, t, J=8.7 Hz), 7.13-7.21 (1H, m), 7.25-7.50 (4H, m), 7.59 (1H, t, J=7.5 Hz), 7.84 (1H, d, J=7.5 Hz), 8.12 (1H, d, J=7.2 Hz), 8.36 (1H, m), 1H unconfirmed.

### Reference Example 287

### (4-Bromo-1-benzofuran-2-yl)(3,4-difluorophenyl)methanone

2-Bromo-6-hydroxybenzaldehyde and 2-bromo-1-(3,4-difluorophenyl)ethanone were used in the same manner as in Reference Example 8 to obtain the titled compound. Yield: 50%.

¹H-NMR (CDCl₃) δ: 7.32-7.42 (2H, m), 7.52 (1H, d, J=7.8 Hz), 7.55-7.62 (2H, m), 7.88-8.01 (2H, m).

### Reference Example 288

### 4-Bromo-2-(3,4-difluorobenzyl)-1-benzofuran

(4-Bromo-1-benzofuran-2-yl)(3,4-difluorophenyl)methanone obtained in Reference Example 287 was used in the same manner as in Reference Example 266 to obtain the titled compound. Yield: 25%.

¹H-NMR (CDCl₃) δ: 4.05 (2H, s), 6.61 (1H, s), 7.11-7.20 (3H, m), 7.31-7.39 (3H, m).

### Reference Example 289

### 3-[2-(3,4-difluorobenzyl)-1-benzofuran-4-yl]benzoic acid

4-Bromo-2-(3,4-difluorobenzyl)-1-benzofuran obtained in Reference Example 288, and [3-(ethoxycarbonyl)phenyl]boronic acid were used in the same manner as in Reference Example 10 to obtain the titled compound. Yield: 64% melting point: 194-195° C. (ethyl acetate-hexane).

¹H-NMR (CDCl₃) δ: 4.08 (2H, s), 6.61 (1H, s), 6.99-7.15 (3H, m), 7.29-7.46 (3H, m), 7.58 (1H, t, J=7.8 Hz), 7.84 (1H, d, J=7.8 Hz), 8.12 (1H, d, J=7.8 Hz), 8.35 (1H, m), 1H unconfirmed.

### Reference Example 290

### Ethyl 3-[2-(4-chloro-3-fluorophenoxy)-1-methyl-1H-benzimidazol-4-yl]benzoate

Ethyl 3-(2-chloro-1-methyl-1H-benzimidazole-4-yl)benzoate obtained in Reference Example 141, and 4-chloro-3-fluorophenol were used in the same manner as in Reference Example 143 to obtain the titled compound. Yield: 62%.

¹H-NMR (CDCl₃) δ: 1.38 (3H, t, J=7.2 Hz), 3.76 (3H, s), 4.39 (2H, q, J=7.2 Hz), 7.22-7.30 (1H, m), 7.39-7.55 (6H, m), 8.00 (1H, d, J=7.5 Hz), 8.16 (1H, d, J=7.5 Hz), 8.56 (1H, t, J=1.8 Hz).

### Reference Example 291

### 3-[2-(4-Chloro-3-fluorophenoxy)-1-methyl-1H-benzimidazol-4-yl]benzoic acid

Ethyl 3-[2-(4-chloro-3-fluorophenoxy)-1-methyl-1H-benzimidazol-4-yl]benzoate obtained in Reference Example 290 was used in the same manner as in Reference Example 233 to obtain the titled compound. Yield: 94%, melting point: 283-284° C. (ethyl acetate-hexane).

¹H-NMR (DMSO-d₆) δ: 3.77 (3H, s), 7.33 (1H, t, J=7.8 Hz), 7.45-7.58 (4H, m), 7.67 (1H, t, J=8.7 Hz), 7.77 (1H, dd, J=10.2, 2.7 Hz), 7.89 (1H, d, J=7.8 Hz), 8.15 (1H, d, J=7.8 Hz), 8.68 (1H, s), 12.9 (1H, br s).

### Reference Example 292

### Ethyl 3-fluoro-5-[2-(hydroxymethyl)-1-benzothiophen-7-yl]benzoate

Ethyl 3-fluoro-5-(4,4,5,5-tetramethyl-1,3,2-dioxaborolane-2-yl)benzoate obtained in Reference Example 225, and (7-bromo-1-benzothiophene-2-yl)methanol synthesized in Reference Example 162 were used in the same manner as in Reference Example 170 to obtain the titled compound. Yield: 71%.

¹H-NMR (CDCl₃) δ: 1.41 (3H, t, J=7.2 Hz), 2.02 (1H, br s), 4.41 (2H, q, J=7.2 Hz), 4.93 (2H, d, J=4.2 Hz), 7.29-7.37 (2H, m), 7.45 (1H, t, J=7.5 Hz), 7.58-7.64 (1H, m), 7.72-7.79 (2H, m), 8.17 (1H, t, J=1.5 Hz).

### Reference Example 293

### Ethyl 3-[2-(3-chloro-4-fluorobenzyl)-1-benzothiophen-7-yl]-5-fluorobenzoate

Ethyl 3-fluoro-5-[2-(hydroxymethyl)-1-benzothiophen-7-yl]benzoate obtained in Reference Example 292, and [3-(ethoxycarbonyl)phenyl]boronic acid was used in the same manner as in Reference Example 197 to obtain the titled compound. Yield: 39%.

¹H-NMR (CDCl₃) δ: 1.39 (3H, t, J=7.2 Hz), 4.16 (2H, s), 4.40 (2H, q, J=7.2 Hz), 7.03-7.19 (3H, m), 7.20-7.31 (2H, m), 7.42 (1H, t, J=7.5 Hz), 7.52-7.60 (1H, m), 7.67-7.77 (2H, m), 8.14 (1H, t, J=1.5 Hz).

### Reference Example 294

### 3-[2-(3-Chloro-4-fluorobenzyl)-1-benzothiophen-7-yl]-5-fluorobenzoic acid

Ethyl 3-[2-(3-chloro-4-fluorobenzyl)-1-benzothiophen-7-yl]-5-fluorobenzoate obtained in Reference Example 293 was used in the same manner as in Reference Example 5 to obtain the titled compound. Yield: 79%, melting point: 135-137 ° C. (ethyl acetate-hexane).

¹H-NMR (CDCl₃) δ: 4.17 (2H, s), 7.02-7.18 (3H, m), 7.27-7.34 (2H, m), 7.43 (1H, t, J=7.5 Hz), 7.64 (1H, dd, J=9.0, 1.5 Hz), 7.71 (1H, d, J=8.1 Hz), 7.80 (1H, d, J=8.7 Hz), 8.22 (1H, s), 1H unconfirmed.

### Reference Example 295

### 3-[2-[3-(Trifluoromethyl)benzyl]-2,3-dihydro-1-benzofuran-4-yl]benzoic acid

Triethylsilane (0.4 mL, 2.5 mmol) was added at room temperature to a trifluoroacetic acid (10 mL) mixture of 3-[2-[3-(trifluoromethyl)benzyl]-1-benzofuran-4-yl]benzoic acid (0.50 g, 1.26 mmol) obtained in Reference Example 10, and the mixture was heated to 60° C. for 16 hours. The reaction solution was concentrated at reduced pressure, and the subsequent addition of saturated sodium bicarbonate aqueous solution to the residue was followed by extraction with ethyl acetate. The organic layer was washed with water and saturated brine, then dried over anhydrous sodium sulfate, and concentrated at reduced pressure to give 0.4 g of the titled compound (yield 79%).

1H-NMR (CDCl₃) δ: 2.98-3.16 (2H, m), 3.19 (1H, dd, J=15.2, 7.5 Hz), 3.39 (1H, dd, J=15.3, 8.7 Hz), 5.00-5.40 (2H, m), 6.81 (1H, d, J=7.8 Hz), 6.93 (1H, d, J=7.8 Hz), 7.21 (1H, t, J=8.1 Hz), 7.38-7.57 (5H, m), 7.66 (1H, d, J=7.8 Hz), 8.08 (1H, d, J=7.5 Hz), 8.19 (1H, s).

### Reference Example 296

### (4-Bromo-1-benzofuran-2-yl)(3-chloro-5-fluorophenyl)methanone

2-Bromo-6-hydroxybenzaldehyde, and 2-bromo-1-(3-chloro-5-fluorophenyl)ethanone were used in the same manner as in Reference Example 8 to obtain the titled compound. Yield: 63%.

¹H-NMR (CDCl₃) δ: 7.34-7.42 (2H, m), 7.52 (1H, dd, J=8.1, 1.2 Hz), 7.52-7.62 (2H, m), 7.68 (1H, ddd, J=8.4, 2.4, 1.2 Hz), 7.85 (1H, s).

### Reference Example 297

### 4-Bromo-2-(3-chloro-5-fluorobenzyl)-1-benzofuran

Triethylsilane (2.7 mL, 17 mmol) was added while cooled on ice to a trifluoroacetic acid (40 mL) solution of (4-bromo-1-benzofuran-2-yl)(3-chloro-5-fluorophenyl)methanone (2.0 g, 5.7 mmol) obtained in Reference Example 296, and the mixture was stirred for 5 hours at room temperature. Ethyl acetate was added to the reaction solution, the mixture was washed with water and saturated brine, and was dried over anhydrous magnesium sulfate, and the solvent was then distilled off at reduced pressure. The resulting residue was purified by silica chromatography (hexane only). The resulting crude product was further washed with hexane to give 0.96 g of the titled compound (yield 50%).

¹H-NMR (CDCl₃) δ: 4.08 (2H, s), 6.51 (1H, s), 6.92 (1H, dt, J=9.0, 1.8 Hz), 7.01 (1H, dt, J=8.5, 2.1 Hz), 7.08-7.11 (1H, m), 7.13 (1H, d, J=8.2 Hz), 7.36 (2H, d, J=8.0 Hz).

### Reference Example 298

### Ethyl 3-[2-(3-chloro-5-fluorobenzyl)-1-benzofuran-4-yl]benzoate

4-Bromo-2-(3-chloro-5-fluorobenzyl)-1-benzofuran obtained in Reference Example 297 was used in the same manner as in Reference Example 4 to obtain the titled compound. Yield: 92%, oily substance

¹H-NMR (CDCl₃) δ: 1.42 (3H, t, J=7.1 Hz), 4.10 (2H, s), 4.42 (2H, q, J=7.1 Hz), 6.64 (1H, s), 6.92 (1H, dt, J=9.1, 1.9 Hz), 6.98 (1H, dt, J=8.3, 2.2 Hz), 7.09 (1H, s), 7.28-7.36 (2H, m), 7.42-7.47 (1H, m), 7.55 (1H, t, J=7.7 Hz), 7.79 (1H, dt, J=7.7, 1.5 Hz), 8.06 (1H, dt, J=7.7, 1.5 Hz), 8.29 (1H, t, J=1.8 Hz).

### Reference Example 299

### 3-[2-(3-Chloro-5-fluorobenzyl)-1-benzofuran-4-yl]benzoic acid

Ethyl 3-[2-(3-chloro-5-fluorobenzyl)-1-benzofuran-4-yl]benzoate obtained in Reference Example 298 was used in the same manner as in Reference Example 5 to obtain the titled compound. Yield: 87%

¹H-NMR (DMSO-d₆) δ: 4.23 (2H, s), 6.84 (1H, s), 7.20 (1H, dd, J=9.5, 1.5 Hz), 7.26-7.45 (4H, m), 7.49-7.60 (1H, m), 7.64 (1H, t, J=7.7 Hz), 7.97 (1H, dd, J=7.7, 1.1 Hz), 7.88 (1H, dd, J=7.7, 1.1 Hz), 8.15 (1H, s), 13.10 (1H, br s).

### Reference Example 300

### Ethyl 3-[2-(3-chloro-4-fluorobenzyl)-3-methyl-1-benzothiophen-7-yl]benzoate

Ethyl 3-[2-(bromomethyl)-3-methyl-1-benzothiophene-7-yl]benzoate obtained in Reference Example 180, and (3-chloro-4-fluorophenyl)boronic acid were used in the same manner as in Reference Example 4 to obtain the titled compound. Yield: 59%

¹H-NMR (CDCl₃) δ: 1.40 (3H, t, J=7.0 Hz), 2.41 (3H, s), 4.16 (2H, s), 4.40 (2H, q, J=7.1 Hz), 6.98-7.13 (2H, m), 7.23 (1H, dd, J=7.0, 2.1 Hz), 7.37 (1H, dd, J=7.1, 1.1 Hz), 7.45-7.59 (2H, m), 7.67 (1H, dd, J=8.0, 1.1 Hz), 7.88 (1H, dt, J=8.0, 1.5 Hz), 8.07 (1H, dt, J=7.8, 1.5 Hz), 8.34 (1H, t, J=1.6 Hz).

### Reference Example 301

### Ethyl 3-[2-(3-acetylbenzyl)-1-benzothiophen-7-yl]benzoate

Ethyl 3-[2-(bromomethyl)-1-benzothiophene-7-yl]benzoate obtained in Reference Example 179, and (3-acetylphenyl)boronic acid were used in the same manner as in Reference Example 4 to obtain the titled compound. Yield: 58%

¹H-NMR (CDCl₃) δ: 1.40 (3H, t, J=7.1 Hz), 2.59 (3H, s), 4.28 (2H, s), 4.40 (2H, q, J=7.1 Hz), 7.11 (1H, s), 7.28-7.36 (1H, m), 7.36-7.46 (2H, m), 7.46-7.50 (1H, m), 7.53 (1H, t, J=7.7 Hz), 7.68 (1H, dd, J=8.0, 1.1 Hz), 7.79-7.92 (3H, m), 8.07 (1H, dt, J=8.0, 1.2 Hz), 8.33 (1H, t, J=1.8 Hz).

### Reference Example 302

### Ethyl 3-[2-[(6-methoxypyridin-3-yl)methyl]-1-benzothiophen-7-yl]benzoate

Ethyl 3-[2-(bromomethyl)-1-benzothiophene-7-yl]benzoate obtained in Reference Example 179, and (6-methoxypyridine-3-yl)boronic acid were used in the same manner as in Reference Example 4 to obtain the titled compound. Yield: 92%

¹H-NMR (CDCl₃) δ: 1.34-1.47 (3H, m), 3.92 (3H, s), 4.14 (2H, s), 4.40 (2H, q, J=7.1 Hz), 6.69 (1H, d, J=8.5 Hz), 7.08 (1H, s), 7.28-7.35 (1H, m), 7.36-7.45 (1H, m), 7.47 (1H, dd, J=8.5, 2.5 Hz), 7.50-7.57 (1H, m), 7.67 (1H, dd, J=8.0, 1.1 Hz), 7.81-7.91 (1H, m), 8.02-8.13 (2H, m), 8.33 (1H, t, J=1.8 Hz).

### Reference Example 303

### Ethyl 3-[2-[(2-methoxypyridin-3-yl)methyl]-1-benzothiophen-7-yl]benzoate

Ethyl 3-[2-(bromomethyl)-1-benzothiophene-7-yl]benzoate obtained in Reference Example 179, and (2-methoxypyridine-3-yl)boronic acid was used in the same manner as in Reference Example 4 to obtain the titled compound. Yield: 91%

¹H-NMR (CDCl₃) δ: 1.31-1.48 (3H, m), 3.97 (3H, s), 4.17 (2H, s), 4.40 (2H, q, J=7.1 Hz), 6.82 (1H, dd, J=7.3, 5.1 Hz), 7.11 (1H, s), 7.29-7.35 (1H, m), 7.38-7.47 (2H, m), 7.54 (1H, t, J=7.7 Hz), 7.68 (1H, dd, J=8.0, 1.1 Hz), 7.89 (1H, dt, J=7.7, 1.5 Hz), 8.01-8.15 (2H, m), 8.35 (1H, t, J=1.5 Hz).

### Reference Example 304

### 3-[2-[(6-Methoxypyridin-3-yl)methyl]-1-benzothiophen-7-yl]benzoic acid

Ethyl 3-[2-[(6-methoxypyridin-3-yl)methyl]-1-benzothiophen-7-yl]benzoate obtained in Reference Example 302 was used in the same manner as in Reference Example 5 to obtain the titled compound. Yield: 98%

¹H-NMR (CDCl₃) δ: 3.92 (3H, s), 4.14 (2H, s), 6.70 (1H, d, J=8.2 Hz), 7.08 (1H, s), 7.29-7.36 (1H, m), 7.42 (1H, t, J=7.5 Hz), 7.48 (1H, dd, J=8.6, 2.3 Hz), 7.58 (1H, t, J=7.7 Hz), 7.67 (1H, d, J=7.7 Hz), 7.93 (1H, d, J=8.0 Hz), 8.04-8.21 (2H, m), 8.41 (1H, s). 1H unconfirmed.

### Reference Example 305

### 3-[2-[(2-Methoxypyridin-3-yl)methyl]-1-benzothiophen-7-yl]benzoic acid

Ethyl 3-[2-[(2-methoxypyridin-3-yl)methyl]-1-benzothiophen-7-yl]benzoate obtained in Reference Example 303 was used in the same manner as in Reference Example 5 to obtain the titled compound. Yield: 97%

¹H-NMR (CDCl₃) δ: 3.98 (3H, s), 4.17 (2H, s), 6.82 (1H, dd, J=7.3, 5.1 Hz), 7.11 (1H, s), 7.29-7.36 (1H, m), 7.36-7.48 (2H, m), 7.58 (1H, t, J=7.7 Hz), 7.68 (1H, dd, J=8.0, 1.1 Hz), 7.94 (1H, dt, J=7.5, 1.6 Hz), 8.06 (1H, dd, J=4.9, 1.9 Hz), 8.13 (1H, dt, J=8.0, 1.2 Hz), 8.43 (1H, t, J 1.5 Hz). 1H unconfirmed

### Reference Example 306

### 3-[2-[(6-Oxo-1,6-dihydropyridin-3-yl)methyl]-1-benzothiophen-7-yl]benzoic acid

Concentrated hydrochloric acid (4 mL) was added to an acetic acid (4 mL) solution of ethyl 3-[2-[(6-methoxypyridin-3-yl)methyl]-1-benzothiophen-7-yl]benzoate (0.20 g, 0.50 mmol) obtained in Reference Example 302, and the mixture was stirred for 19 hours at 100° C. The reaction solution was poured into water, and the precipitate was then filtered off to give 0.16 g of the titled compound (yield 90%).

¹H-NMR (DMSO-d₆) δ: 4.01 (2H, s), 6.34 (1H, d, J=10.2 Hz), 7.29 (1H, s), 7.33-7.57 (5H, m), 7.57-7.73 (1H, m), 7.79 (1H, d, J=7.7 Hz), 7.90 (1H, d, J=8.0 Hz), 7.99 (1H, d, J=7.7 Hz), 8.20 (1H, d, J=1.6 Hz). 1H unconfirmed.

### Reference Example 307

### 3-[2-[(2-Oxo-1,2-dihydropyridin-3-yl)methyl]-1-benzothiophen-7-yl]benzoic acid

Ethyl 3-[2-[(2-methoxypyridin-3-yl)methyl]-1-benzothiophen-7-yl]benzoate obtained in Reference Example 303 was used in the same manner as in Reference Example 306 to obtain the titled compound. Yield: 94%

¹H-NMR (DMSO-d₆) δ: 3.99 (2H, s), 6.12 (1H, t, J=6.5 Hz), 7.22-7.30 (2H, m), 7.32-7.41 (2H, m), 7.41-7.49 (1H, m), 7.59-7.70 (1H, m), 7.77 (1H, d, J=6.9 Hz), 7.89 (1H, d, J=7.7 Hz), 7.99 (1H, d, J=7.7 Hz), 8.19 (1H, t, J=1.8 Hz). 2H unconfirmed.

### Reference Example 308

### Ethyl 3-[2-[(6-oxo-1,6-dihydropyridin-3-yl)methyl]-1-benzothiophen-7-yl]benzoate

A hydrogen chloride-ethanol (5 mL) solution of ethyl 3-[2-[(6-methoxypyridin-3-yl)methyl]-1-benzothiophen-7-yl]benzoate (0.30 g, 0.74 mmol) obtained in Reference Example 302 was stirred for 24 hours at 100° C. The addition of water to the reaction solution was followed by extraction with ethyl acetate. The extract was washed with saturated sodium bicarbonate aqueous solution and saturated brine, and was then dried over anhydrous magnesium sulfate, and the solvent was distilled off at reduced pressure. The resulting residue was washed with hexane-ethyl acetate (5:1) to give 0.23 g of the titled compound (yield 80%).

¹H-NMR (CDCl₃) δ: 1.40 (3H, t, J=7.1 Hz), 3.97 (2H, s), 4.40 (2H, q, J=7.1 Hz), 6.54 (1H, d, J=9.6 Hz), 7.12 (1H, s), 7.19 (1H, d, J=1.6 Hz), 7.31-7.35 (1H, m), 7.38 (1H, dd, J=9.3, 2.5 Hz), 7.44 (1H, t, J=7.5 Hz), 7.51-7.59 (1H, m), 7.69 (1H, dd, J=8.0, 1.1 Hz), 7.87 (1H, dt, J=7.8, 1.3 Hz), 8.08 (1H, dt, J=7.9, 1.4 Hz), 8.33 (1H, t, J=1.8 Hz), 1H unconfirmed.

### Reference Example 309

### Ethyl 3-[2-[(1-methyl-6-oxo-1,6-dihydropyridin-3-yl)methyl]-1-benzothiophen-7-yl]benzoate

Potassium carbonate (0.18 g, 1.3 mmol) and methyl iodide (82 μL, 1.3 mmol) were added to a DMF (10 mL) solution of ethyl 3-[2-[(6-oxo-1,6-dihydropyridin-3-yl)methyl]-1-benzothiophen-7-yl]benzoate (0.23 g, 0.60 mmol) obtained in Reference Example 308, and the mixture was stirred for 1 hour at room temperature and then stirred for 2 hours at 50° C. The addition of water to the reaction solution was followed by extraction with ethyl acetate. The extract was washed with water and saturated brine, and was dried over anhydrous magnesium sulfate, and the solvent was then distilled off at reduced pressure. The resulting residue was purified by silica gel chromatography (hexane-ethyl acetate 3:7→ethyl acetate alone→ethyl acetate-methanol=9:1) to give 0.18 g of the titled compound (yield 74%).

¹H-NMR (CDCl₃) δ: 1.41 (3H, t, J=7.1 Hz), 3.52 (3H, s), 3.95 (2H, s), 4.41 (2H, q, J=7.1 Hz), 6.55 (1H, d, J=9.1 Hz), 7.13 (1H, s), 7.16 (1H, d, J=1.9 Hz), 7.23-7.30 (H, m), 7.32-7.37 (1H, m), 7.45 (1H, t, J=7.6 Hz), 7.56 (1H, t, J=8.0 Hz), 7.70 (1H, dd, J=7.7, 1.1 Hz), 7.87 (1H, dt, J=7.7, 1.5 Hz), 8.08 (1H, dt, J=7.9, 1.4 Hz), 8.34 (1H, t, J=1.5 Hz).

### Reference Example 310

### 3-[2-[(1-Methyl-6-oxo-1,6-dihydropyridin-3-yl)methyl]-1-benzothiophen-7-yl]benzoic acid

Ethyl 3-[2-[(1-methyl-6-oxo-1,6-dihydropyridin-3-yl)methyl]-1-benzothiophen-7-yl]benzoate obtained in Reference Example 309 was used in the same manner as in Reference Example 5 to obtain the titled compound. Yield: 93%

¹H-NMR (DMSO-d₆) δ: 3.37 (3H, s), 3.98 (2H, s), 6.33 (1H, d, J=9.1 Hz), 7.26-7.41 (3H, m), 7.46 (1H, t, J=7.6 Hz), 7.57-7.73 (2H, m), 7.78 (1H, d, J=7.7 Hz), 7.89 (1H, dd, J=7.7, 1.1 Hz), 7.99 (1H, dd, J=7.8, 1.2 Hz), 8.20 (1H, s). 1H unconfirmed.

### Reference Example 311

### Ethyl 3-[2-[(1-methyl-2-oxo-1,2-dihydropyridin-3-yl)methyl]-1-benzothiophen-7-yl]benzoate

A hydrogen chloride-methanol (5 mL) solution of ethyl 3-[2-[(2-methoxypyridin-3-yl)methyl]-1-benzothiophen-7-yl]benzoate (0.33 g, 0.81 mmol) obtained in Reference Example 303 was stirred for 15 hours at 100° C., and the solvent was distilled off at reduced pressure. Methyl iodide (87 μL, 1.8 mmol) was added to a DMF (10 mL) solution of the resulting residue and cesium carbonate (0.58 g, 1.8 mmol), and the mixture was stirred for 2 hours. The addition of water to the reaction solution was followed by extraction with ethyl acetate. The extract was washed with water and saturated brine, and was dried over anhydrous magnesium sulfate, and the solvent was then distilled off at reduced pressure. The resulting residue was purified by silica chromatography to give 0.27 g of the titled compound (yield 84%).

¹H-NMR (CDCl₃) δ: 1.40 (3H, t, J=7.0 Hz), 3.56 (3H, s), 4.14 (2H, s), 4.40 (2H, q, J=7.1 Hz), 6.09 (1H, t, J=6.7 Hz), 7.14-7.25 (3H, m), 7.28-7.34 (1H, m), 7.36-7.46 (1H, m), 7.54 (1H, t, J=7.7 Hz), 7.69 (1H, dd, J=7.7, 1.1 Hz), 7.90 (1H, ddd, J=7.7, 1.9, 1.1 Hz), 8.07 (1H, dt, J=7.8, 1.5 Hz), 8.34 (1H, t, J=1.8 Hz).

### Reference Example 312

### 3-[2-[(1-Methyl-2-oxo-1,2-dihydropyridin-3-yl)methyl]-1-benzothiophen-7-yl]benzoic acid

Ethyl 3-[2-[(1-methyl-2-oxo-1,2-dihydropyridin-3-yl)methyl]-1-benzothiophen-7-yl]benzoate obtained in Reference Example 311 was used in the same manner as in Reference Example 5 to obtain the titled compound. Yield: 95%

¹H-NMR (DMSO-d₆) δ: 3.41 (3H, s), 4.02 (2H, s), 6.15 (1H, t, J=6.7 Hz), 7.29 (1H, s), 7.31-7.36 (1H, m), 7.38 (1H, dd, J=6.9, 1.9 Hz), 7.41-7.47 (1H, m), 7.59 (1H, dd, J=6.6, 1.9 Hz), 7.64 (1H, t, J=7.7 Hz), 7.73-7.79 (1H, m), 7.89 (1H, d, J=7.7 Hz), 7.99 (1H, d, J=7.7 Hz), 8.19 (1H, t, J=1.8 Hz), 13.14 (1H, br s).

### Reference Example 314 and Reference Example 315

### Ethyl 3-[2-[[1-(1-methylethyl)-6-oxo-1,6-dihydropyridin-3-yl]methyl]-1-benzothiophen-7-yl]benzoate and Ethyl 3-[2-[[6-(1-methylethoxy)pyridin-3-yl]methyl]-1-benzothiophen-7-yl]benzoate

Isopropyl iodide (0.18 mL, 1.84 mmol) and cesium carbonate (0.82 g, 2.82 mmol) were added to a DMF (10 mL) solution of ethyl

3-[2-[(6-oxo-1,6-dihydropyridin-3-yl)methyl]-1-benzothiophen-7-yl]benzoate (0.55 g, 1.41 mmol) obtained in Reference Example 308, and the mixture was stirred for 1 hour at 50° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:methanol=5:1, ethyl acetate) to give ethyl 3-[2-[[6-(1-methylethoxy)pyridin-3-yl]methyl]-1-benzothiophen-7-yl]benzoate (0.42 g, 69%) in the form of a colorless oily substance as the first distillate. ¹H NMR (CDCl₃) δ: 1.33 (6H, d, J=6.3 Hz), 1.39 (3H, t, J=7.2 Hz), 4.11 (2H, s), 4.40 (2H, q, J=7.2 Hz), 5.20-5.35 (1H, m), 6.62 (1H, d, J=8.7 Hz), 7.08 (1H, s), 7.31 (1H, d, J=7.2 Hz), 7.41 (1H, t, J=7.8 Hz), 7.44 (1H, dd, J=8.1, 2.7 Hz), 7.53 (1H, t, J=7.8 Hz), 7.66 (1H, d, J=7.8 Hz), 7.87 (1H, d, J=7.5 Hz), 8.00-8.10 (2H, m), 8.33 (1H, s).

Ethyl 3-[2-[[1-(1-methylethyl)-6-oxo-1,6-dihydropyridin-3-yl]methyl]-1-benzothiophen-7-yl]benzoate (0.167 g, 27%) was also obtained in the form of a colorless oily substance as the second distillate. ¹H NMR (CDCl₃) δ: 1.35 (6H, d, J=6.3 Hz), 1.40 (3H, t, J=7.2 Hz), 3.97 (2H, s), 4.40 (2H, q, J=7.2 Hz), 5.20-5.35 (1H, m), 6.53 (1H, d, J=9.9 Hz), 7.10 (1H, s), 7.20-7.35 (2H, m), 7.33 (1H, d, J=7.2 Hz), 7.44 (1H, t, J=7.2 Hz), 7.55 (1H, t, J=7.5 Hz), 7.69 (1H, d, J=7.5 Hz), 7.87 (1H, d, J=7.2 Hz), 8.07 (1H, d, J=6.6 Hz), 8.34 (1H, s).

### Reference Example 316 and Reference Example 317

### Ethyl 3-[2-[[6-oxo-1-(2,2,2-trifluoroethyl)-1,6-dihydropyridin-3-yl]methyl]-1-benzothiophen-7-yl]benzoate and Ethyl 3-[2-[[6-(2,2,2-trifluoroethoxy)pyridin-3-yl]methyl]-1-benzothiophen-7-yl]benzoate

2,2,2-Trifluoroethyl trichloromethane sulfonate (0.54 g, 1.93 mmol) and cesium carbonate (0.84 g, 2.56 mmol) were added to a DMF (7 mL) solution of ethyl 3-[2-[(6-oxo-1,6-dihydropyridin-3-yl)methyl]-1-benzothiophen-7-yl]benzoate (0.5 g, 1.28 mmol) obtained in Reference Example 308, and the mixture was stirred for 4 hour at room temperature. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:methanol=10:1 to 1:1) to give ethyl 3-[2-[[6-(2,2,2-trifluoroethoxy)pyridin-3-yl]methyl]-1-benzothiophen-7-yl]benzoate (0.33 g, 55%) in the form of a colorless oily substance as the first distillate. ¹H NMR (CDCl₃) δ: 1.40 (3H, t, J=7.2 Hz), 4.16 (2H, s), 4.40 (2H, q, J=6.9 Hz), 4.73 (2H, q, J=8.1 Hz), 6.81 (1H, d, J=8.7 Hz), 7.09 (1H, s), 7.38 (1H, d, J=7.5 Hz), 7.42 (1H, t, J=7.5 Hz), 7.50-7.60 (2H, m), 7.68 (1H, d, J=7.8 Hz), 7.86 (1H, d, J=7.5 Hz), 8.00-8.10 (2H, m), 8.32 (1H, s).

Ethyl 3-[2-[[6-oxo-1-(2,2,2-trifluoroethyl)-1,6-dihydropyridin-3-yl]methyl]-1-benzothiophen-7-yl]benzoate (0.20 g, 33%) was also obtained in the form of a colorless oily substance as the second distillate. ¹H NMR (CDCl₃) δ: 1.40 (3H, t, J=7.1 Hz), 3.96 (2H, s), 4.40 (2H, q, J=6.8 Hz), 4.57 (2H, q, J=8.5 Hz), 6.58 (1H, d, J=8.7 Hz), 7.13 (2H, s), 7.29 (1H, dd, J=8.7, 1.5 Hz), 7.34 (1H, q, J=7.2 Hz), 7.47 (1H, t, J=7.5 Hz), 7.55 (1H, t, J=7.8 Hz), 7.70 (1H, d, J=8.1 Hz), 7.86 (1H, d, J=8.1 Hz), 8.33 (1H, s).

### Reference Example 318

### 5-Bromo-1-methyl-3-(trifluoromethyl)pyridin-2(1H)-one

Methyl iodide (0.55 mL, 8.76 mmol) and cesium carbonate (4.39 g, 13.5 mmol) were added to a DMF (15 mL) solution of 5-bromo-3-(trifluoromethyl)pyridin-2(1H)-one (1.63 g, 6.74 mmol), and the mixture was stirred for 1 hour. The reaction solution was diluted with water and extracted with ethyl acetate. The extract was washed with water and dried over anhydrous magnesium sulfate, and the solvent was then distilled off at reduced pressure. The residue was crystallized from hexane-diethyl ether to give 1.47 g of the titled compound (yield 85%).

¹H NMR (CDCl₃) δ: 3.59 (3H, s), 7.63 (1H, d, J=3.3 Hz), 7.78 (1H, s).

### Reference Example 319

### Ethyl 3-[2-[[1-methyl-6-oxo-5-(trifluoromethyl)-1,6-dihydropyridin-3-yl]methyl]-1-benzothiophen-7-yl]benzoate

A mixture of 5-bromo-1-methyl-3-(trifluoromethyl)pyridin-2(1H)-one (0.5 g, 1.95 mmol) obtained in Reference Example 318, bis(pinacolato)diborane (0.74 g, 2.93 mmol), [1,1′-bis(diphenylphosphino)ferrocene]dichloropalladium (II) complex with dichloromethane (80 mg, 0.098 mmol), potassium acetate (0.39 g, 3.95 mmol), and DMF (3 mL) was stirred for 4 hours at 80° C. The reaction solution was diluted with 0.5 N hydrochloric acid (30 mL) and extracted with ethyl acetate. The extract was washed with water and dried over anhydrous magnesium sulfate, and the solvent was then distilled off at reduced pressure. Ethyl 3-[2-(bromomethyl)-1-benzothiophen-7-yl]benzoate (0.41 g, 1.09 mmol) obtained in Reference Example 179, tetrakis(triphenylphosphine)palladium (0) (54 mg, 0.038 mmol), and sodium carbonate (0.33 g, 3.12 mmol) were added to a 1,2-dimethoxyethane and water mixture (4:1, 25 mL) of the residue, and the mixture was stirred for 6 hours at 70° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=1:1) to give 0.19 g of the titled compound (yield 37%) in the form of an oily substance.

¹H NMR (CDCl₃) δ: 1.42 (3H, t, J=7.2 Hz), 3.56 (3H, s), 4.00 (2H, s), 4.40 (2H, q, J=7.2 Hz), 7.15 (1H, s), 7.15-7.20 (2H, m), 7.46 (1H, t, J=7.7 Hz), 7.56 (1H, t, J=7.7 Hz), 7.66 (1H, s), 7.72 (1H, d, J=7.8 Hz), 7.86 (1H, d, J=7.2 Hz), 8.09 (1H, d, J=7.5 Hz), 8.34 (1H, s).

### Reference Example 320

### 5-Bromo-3-chloropyridin-2(1H)-one

A mixture of 5-bromo-2,3-dichloropyridine (1.0 g, 4.41 mmol), sodium hydroxide (0.53 g, 13.2 mmol), water (5 mL), and DMSO (5 mL) was stirred for 2 hours at 100° C. The addition of 1 N hydrochloric acid (30 mL) to the reaction solution was followed by extraction with ethyl acetate. The extract was washed with water and dried over anhydrous magnesium sulfate, and the solvent was then distilled off at reduced pressure to give 0.83 g of the titled compound (yield 90%) in the form of crystals.

¹H NMR (CDCl₃) δ: 7.48 (1H, d, J=2.4 Hz), 7.72 (1H, d, J=2.7 Hz).

### Reference Example 321

### 5-Bromo-3-chloro-1-methylpyridin-2(1H)-one

5-Bromo-3-chloropyridin-2(1H)-one obtained in Reference Example 320 (0.82 g, 3.93 mmol) was dissolved in DMF (10 mL), methyl iodide (0.32 mL, 5.11 mmol) and cesium carbonate (2.56 g, 7.87 mmol) were added, and the mixture was stirred for 30 min. The reaction solution was diluted with water and extracted with ethyl acetate. The extract was washed with water and dried over anhydrous magnesium sulfate, and the solvent was then distilled off at reduced pressure. The residue was crystallized from hexane to give 0.76 g of the titled compound (yield 87%).

¹H NMR (CDCl₃) δ: 3.59 (3H, s), 7.38 (1H, d, J=2.4 Hz), 7.59 (1H, d, J=3.3 Hz).

### Reference Example 322

### Ethyl 3-[2-[(5-chloro-1-methyl-6-oxo-1,6-dihydropyridin-3-yl)methyl]-1-benzothiophen-7-yl]benzoate

A mixture of5-bromo-3-chloro-1-methylpyridin-2(1H)-one (0.6 g, 2.70 mmol) obtained in Reference Example 321, bis(pinacolato)diborane (1.03 g, 4.05 mmol), [1,1′-bis(diphenylphosphino)ferrocene]dichloropalladium (II) complex with dichloromethane (0.11 g, 0.13 mmol), potassium acetate (0.40 g, 4.05 mmol), and DMF (10 mL) was stirred for 1.5 hours at 70° C. The reaction solution was diluted with 1 N hydrochloric acid (10 mL) and extracted with ethyl acetate. The extract was washed with water and dried over anhydrous magnesium sulfate, and the solvent was then distilled off at reduced pressure. Ethyl 3-[2-(bromomethyl)-1-benzothiophen-7-yl]benzoate (0.90 g, 2.40 mmol) obtained in Reference Example 179, tetrakis(triphenylphosphine)palladium (0) (156 mg, 0.135 mmol), and sodium carbonate (0.86 g, 8.09 mmol) were added to a 1,2-dimethoxyethane and water mixture (2:1, 15 mL) of the residue, and the mixture was stirred for 5 hours at 75° C. The reaction solution was diluted with water and extracted with ethyl acetate. The extract was washed with water and dried over anhydrous magnesium sulfate, and the solvent was then distilled off at reduced pressure.

The residue was purified by silica gel column chromatography (hexane:ethyl acetate=2:3) to give 0.54 g of the titled compound (yield 51%) in the form of an oily substance.

¹H NMR (CDCl₃) δ: 1.40 (3H, t, J=7.1 Hz), 3.58 (3H, s), 3.95 (2H, s), 4.40 (2H, q, J=7.1 Hz), 7.19 (1H, s), 735 (1H, d, J=6.3 Hz), 7.40-7.70 (4H, m), 7.71 (1H, d, J=8.7 Hz), 7.87 (12H, d, J=8.1 Hz), 8.08 (1H, d, J=7.8 Hz), 8.36 (1H, s).

### Reference Example 323

### Ethyl 3-[2-[(2-methoxypyridin-4-yl)methyl]-1-benzothiophen-7-yl]benzoate

A mixture of 4-bromo-2-methoxypyridine (0.50 g, 2.66 mmol), bis(pinacolato)diborane (1.01 g, 3.99 mmol), [1,1′-bis(diphenylphosphino)ferrocene]dichloropalladium (II) complex with dichloromethane (0.11 g, 0.13 mmol), potassium acetate (0.39 g, 3.99 mmol), and DMF (7 mL) was stirred for 1.5 hours at 70° C. 1 N hydrochloric acid (4 mL) was added to the reaction solution, and the mixture was diluted with water and then extracted with ethyl acetate. The extract was washed with water and dried over anhydrous magnesium sulfate, and the solvent was then distilled off at reduced pressure. Ethyl 3-[2-(bromomethyl)-1-benzothiophen-7-yl]benzoate (0.67 g, 1.77 mmol) obtained in Reference Example 179, tetrakis(triphenylphosphine)palladium (0) (61.5 mg, 0.053 mmol), and sodium carbonate (0.38 g, 3.55 mmol) were added to a dimethoxy ether and water mixture (2:1, 15 mL) of the residue, and the mixture was stirred for 2 hours at 75° C. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=5:1 to 3:1) to give 0.07 g of the titled compound (yield 10%) in the form of an oily substance.

¹H NMR (CDCl₃) δ: 1.40 (3H, t, J=7.2 Hz), 3.91 (3H, s), 4.15 (2H, s), 4.40 (2H, q, J=7.2 Hz), 6.64 (1H, s), 6.79 (1H, d, J=5.4 Hz), 7.13 (1H, s), 7.33 (1H, d, J=7.2 Hz), 7.43 (1H, t, J=7.6 Hz), 7.54 (1H, t, J=7.6 Hz), 7.68 (1H, d, J=7.5 Hz), 7.87 (1H, d, J=7.8 Hz), 8.00-8.10 (2H, m), 8.33 (1H, s).

### Reference Example 324

### Ethyl 3-[2-[(1-methyl-2-oxo-1,2-dihydropyridin-4-yl)methyl]-1-benzothiophen-7-yl]benzoate

A mixture of ethyl 3-[2-[(2-methoxypyridin-4-yl)methyl]-1-benzothiophen-7-yl]benzoate (70 mg, 0.17 mmol) obtained in Reference Example 323 and 15% hydrogen chloride-ethanol solution (10 mL) was heated to reflux for 16 hours. The solvent was distilled off from the reaction solution at reduced pressure, and the residue was dissolved in DMF (3 mL). Methyl iodide (0.016 mL, 0.26 mmol) and cesium carbonate (113 mg, 0.35 mmol) were added, and the mixture was stirred for 5 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane) to give 20.8 mg of the titled compound (yield 30%) in the form of an oily substance.

¹H NMR (CDCl₃) δ: 1.40 (3H, t, J=7.2 Hz), 3.50 (3H, s), 4.01 (2H, s), 4.40 (2H, q, J=7.2 Hz), 6.00-6.10 (1H, m), 6.49 (1H, s), 7.10-7.20 (2H, m), 7.33 (1H, d, J=7.5 Hz), 7.43 (1H, t, J=7.8 Hz), 7.50-7.70 (1H, m), 7.69 (1H, d, J=7.8 Hz), 7.88 (1H, d, J=7.8 Hz), 8.00-8.10 (1H, m), 8.32 (1H, s).

### Reference Example 325

### Methyl 3-(1-methyl-2-[methyl[3-(trifluoromethyl)phenyl]amino]-1H-benzimidazol-4-yl]benzoate

Sodium hydride (58.8 mg, 1.47 mmol) was added while cooled on ice to a DMF (2 mL) solution of 3-[1-methyl-2-[[3-(trifluoromethyl)phenyl]amino]-1H-benzimidazol-4-yl]benzoic acid (200 mg, 0.49 mmol) obtained in Reference Example 233, and the mixture was stirred for 2 hours at room temperature. Iodomethane (152 μg, 2.45 mmol) was added dropwise to the reaction solution, and the mixture was then stirred for 14 hours at room temperature. The reaction solution was treated with the addition of water and was extracted with ethyl acetate. The combined organic layers were washed with water, dried over anhydrous magnesium sulfate, and then concentrated at reduced pressure. The residue was purified by silica gel column chromatography (hexane-ethyl acetate 100:0→80:20) to give 130 mg of the titled compound (yield 60%) in the form of crystals. Melting point: 148-149° C. (ethyl acetate).

¹H-NMR (CDCl₃) δ: 3.38 (3H, s), 3.64 (3H, s), 3.94 (3H, s), 7.00 (1H, dd, J=8.1, 2.1 Hz), 7.17 (1H, s), 7.21-7.28 (2H, m), 7.37 (2H, q, J=8.0 Hz), 7.50 (1H, dd, J=7.5, 1.1 Hz), 7.55 (1H, t, J=7.7 Hz), 7.99-8.06 (1H, m), 8.31-8.39 (1H, m), 8.70 (1H, t, J=1.6 Hz).

### Reference Example 326

### 3-(1-Methyl-2-[methyl[3-(trifluoromethyl)phenyl]amino]-1H-benzimidazol-4-yl)benzoic acid

Methyl 3-(1-methyl-2-[methyl[3-(trifluoromethyl)phenyl]amino]-1H-benzimidazol-4-yl)benzoate obtained in Reference Example 325 was used in the same manner as in Reference Example 5 to obtain the titled compound. Yield: 71% melting point: 211-212° C. (ethyl acetate).

¹H-NMR (DMSO-d₆) δ: 3.49 (3H, s), 3.50 (3H, s), 7.10 (1H, dd, J=8.0, 2.3 Hz), 7.22 (1H, s), 7.29 (1H, d, J=7.6 Hz), 7.36 (1H, t, J=7.8 Hz), 7.45-7.55 (3H, m), 7.59 (1H, t, J=7.8 Hz), 7.92 (1H, d, J=8.0 Hz), 8.31 (1H, d, J=8.3 Hz), 8.60 (1H, s), 12.97 (1H, br s).

### Reference Example 327

### Ethyl 3-(2-[[6-(trifluoromethyl)pyridin-2-yl]methyl]-1-benzothiophen-4-yl)benzoate

Ethyl 3-[2-(hydroxymethyl)-1-benzothiophene-4-yl]benzoate obtained in Reference Example 167, and phosphorus tribromide were used in the same manner as in Reference Example 197 to give ethyl 3-[2-(bromomethyl)-1-benzothiophene-4-yl]benzoate crude product, and the resulting crude preparation of ethyl 3-[2-(bromomethyl)-1-benzothiophene-4-yl]benzoate, and 2-(4,4,5,5-tetramethyl-1,3,2-dioxaborolane-2-yl)-6-(trifluoromethyl)pyridine were used to obtain the, titled compound. Yield: 62% (2 steps).

¹H-NMR (CDCl₃) δ: 1.40 (3H, t, J=7.2 Hz), 4.41 (2H, q, J=7.2 Hz), 4.48 (2H, s), 7.24 (1H, s), 7.31-7.38 (2H, m), 7.40 (1H, d, J=8.0 Hz), 7.55 (2H, t, J=6.8 Hz), 7.71-7.81 (3H, m), 8.08 (1H, d, J=8.0 Hz), 8.23 (1H, s).

### Reference Example 328

### 3-(2-[[6-(Trifluoromethyl)pyridin-2-yl]methyl]-1-benzothiophen-4-yl)benzoic acid

Ethyl 3-(2-[[6-(trifluoromethyl)pyridin-2-yl]methyl]-1-benzothiophen-4-yl)benzoate obtained in Reference Example 327 was used in the same manner as in Reference Example 5 to obtain the titled compound. Yield: 92%, amorphous solid.

¹H-NMR (CDCl₃) δ: 4.48 (2H, s), 7.26 (1H, s), 7.31-7.39 (2H, m), 7.39-7.44 (1H, m), 7.55 (1H, d, J=7.5 Hz), 7.60 (1H, t, J=7.8 Hz), 7.74-7.84 (3H, m), 8.11-8.19 (1H, m), 8.30 (1H, t, J=1.6 Hz), 1H unconfirmed.

### Reference Example 329

### Ethyl 3-(2-[[6-(trifluoromethyl)pyridin-2-yl]methyl]-1-benzothiophen-7-yl)benzoate

Ethyl 3-[2-(bromomethyl)-1-benzothiophene-7-yl]benzoate obtained in Reference Example 179, and 2-(4,4,5,5-tetramethyl-1,3,2-dioxaborolane-2-yl)-6-(trifluoromethyl)pyridine were used in the same manner as in Reference Example 4 to obtain the titled compound. Yield: 50%.

¹H-NMR (CDCl₃) δ: 1.39 (3H, t, J=7.1 Hz), 4.40 (2H, q, J=7.0 Hz), 4.49 (2H, s), 7.23 (1H, s), 7.32-7.37 (1H, m), 7.39-7.48 (2H, m), 7.51-7.59 (2H, m), 7.71 (1H, dd, J=7.9, 1.1 Hz), 7.77 (1H, t, J=7.8 Hz), 7.85-7.92 (1H, m), 8.04-8.11 (1H, m), 8.35 (1H, t, J=1.6 Hz).

### Reference Example 330

### 3-(2-[[6-(Trifluoromethyl)pyridin-2-yl]methyl]-1-benzothiophen-7-yl)benzoic acid

Ethyl 3-(2-[[6-(trifluoromethyl)pyridin-2-yl]methyl]-1-benzothiophen-7-yl)benzoate obtained in Reference Example 329 was used in the same manner as in Reference Example 5 to obtain the titled compound. Yield: 73%.

¹H-NMR (DMSO-d₆) δ: 4.53 (2H, s), 7.37-7.42 (2H, m), 7.49 (1H, t, J=7.6 Hz), 7.64 (1H, t, J=7.7 Hz), 7.72 (1H, d, J=7.9 Hz), 7.78 (1H, d, J=7.5 Hz), 7.82 (1H, dd, J=7.8, 1.0 Hz), 7.88 (1H, d, J=7.7 Hz), 7.96-8.09 (2H, m), 8.24 (1H, s), 13.28 (1H, br s).

### Reference Example 331

### 7-Bromopyrazolo[1,5-a]pyridin-2-ol

An n-butyllithium-hexane solution (2.5 M, 22 mL, 55.9 mmol) was added dropwise at −78° C. to a THF (80 mL) solution of pyrazolo[1,5-a]pyridin-2-ol (1.50 g, 11.2 mmol) and N,N,N′N′-tetramethylethylenediamine (1.69 mL, 11.2 mmol), and the mixture was stirred for 5 min. A THF (30 mL) solution of 1,2-dibromo-1,1,2,2-tetrachloroethane (4.37 g, 13.4 mmol) was added dropwise at −78° C. to the reaction solution, and the mixture was stirred for 30 min. The mixture was heated to room temperature, then diluted with ethyl acetate, and washed with 10% citric acid aqueous solution. The organic layer was dried over anhydrous sodium sulfate and then concentrated at reduced pressure. The residue was purified by silica gel column chromatography (hexane-ethyl acetate 3:1) to give 1.38 g of the titled compound (yield 58%) in the form of crystals.

¹H-NMR (CDCl₃) δ: 6.04 (1H s), 6.96 (2H, m), 7.33 (1H, m), 10.34 (1H, br s).

### Reference Example 332

### Ethyl 3-(2-hydroxypyrazolo[1,5-a]pyridin-7-yl)benzoate

A water (7.00 mL)-toluene (14.0 mL) mixture of 7-bromopyrazolo[1,5-a]pyridin-2-ol (1.35 g, 6.34 mmol) obtained in Reference Example 331, [3-(ethoxycarbonyl)phenyl]boronic acid (1.84 g, 9.51 mmol), tetrakis(triphenylphosphine)palladium (0) (0.366 g, 0.317 mmol), and sodium carbonate (1.34 g, 12.7 mmol) was heated and stirred for 18 hours at 100° C. in a nitrogen atmosphere. After being allowed to cool to room temperature, the reaction solution was diluted with ethyl acetate and washed with 10% citric acid aqueous solution. The organic layer was dried over anhydrous sodium sulfate and then concentrated at reduced pressure. The residue was purified by silica gel column chromatography (hexane-ethyl acetate 5:1) to give 1.34 g of the titled compound (yield 75%) in the form of crystals.

¹H-NMR (CDCl₃) δ: 1.34 (3H, t, J=7.2 Hz), 4.33 (2H, m), 5.83 (1H, s), 6.67 (1H, dd, J=6.8, 1.2 Hz), 7.16 (1H, dd, J=7.2, 8.8 Hz), 7.32 (1H, dd, J=8.8, 1.6 Hz), 7.59 (1H, t, J=7.6 Hz), 8.00 (1H, ddd, J=1.4, 1.6, 7.8 Hz), 8.17 (1H, ddd, J=1.2, 1.4, 8.0 Hz), 8.47 (1H, t, J=1.6 Hz), 1H unconfirmed.

### Reference Example 333

### Ethyl 3-(2-[[3-(trifluoromethyl)benzyl]oxy]pyrazolo[1,5-a]pyridin-7-yl]benzoate

An acetone (12 mL) solution of ethyl 3-(2-hydroxypyrazolo[1,5-a]pyridin-7-yl)benzoate (1.00 g, 3.54 mmol) obtained in Reference Example 332, 1-(bromomethyl)-3-(trifluoromethyl)benzene (0.650 mL, 4.25 mmol), and potassium carbonate (0.587 g, 4.25 mmol) was heated to reflux for 18 hours. The reaction solution was concentrated at reduced pressure, diluted with ethyl acetate, and washed with saturated brine. The organic layer was dried over anhydrous sodium sulfate and then concentrated at reduced pressure. The residue was purified by silica gel column chromatography (hexane-dichloromethane 7:1) to give 0.510 g of the titled compound (yield 33%) in the form of crystals.

¹H-NMR (CDCl₃) δ: 1.40 (3H, t, J=7.0 Hz), 4.41 (2H, m), 5.33 (2H, s), 5.98 (1H, s), 6.75 (1H, dd, J=1.6, 7.0 Hz), 7.16 (1H, dd, J=7.0, 9.0 Hz), 7.34 (1H, dd, J=1.6, 8.8 Hz), 7.47 (1H, m), 7.57 (2H, dd, J=7.6, 8.0 Hz), 7.64 (1H, d, J=7.6 Hz), 7.73 (1H, s), 8.14 (2H, m), 8.56 (1H, m).

### Reference Example 334

### 3-(2-[[3-(Trifluoromethyl)benzyl]oxy]pyrazolo[1,5-a]pyridin-7-yl]benzoate

Sodium hydroxide (0.0230 g, 0.568 mmol) and several drops of water were added at room temperature to an ethanol (4 mL) solution of ethyl 3-(2-[[3-(trifluoromethyl)benzyl]oxy]pyrazolo[1,5-a]pyridin-7-yl]benzoate (0.500 g, 1.14 mmol) obtained in Reference Example 333, and the mixture was then stirred for 4 hours at 45° C. The reaction solution was diluted with water and neutralized with 10% citric acid aqueous solution. The resulting crystals were filtered off, washed with water and hexane, and then dried at reduced pressure to give 0.310 g of the titled compound (yield 66%) in the form of crystals.

¹H-NMR (DMSO-d₆) δ: 5.35 (2H, s), 6.21 (1H, s), 6.94 (1H, dd, J=1.6, 7.0 Hz), 7.29 (1H, dd, J=6.8, 8.8 Hz), 7.53 (1H, dd, J=1.2, 8.8 Hz), 7.62 (2H, m), 7.69 (1H, d, J=7.6 Hz), 7.80 (2H, m), 8.06 (2H, m), 8.46 (1H, t, J=1.6 Hz), 1H unconfirmed.

### Reference Example 335

### Ethyl 3-fluoro-5-[2-(hydroxymethyl)-3-methyl-1-benzothiophen-7-yl]benzoate

(7-Bromo-3-methyl-1-benzothiophene-2-yl)methanol obtained in Reference Example 163, and ethyl 3-fluoro-5-(4,4,5,5-tetramethyl-1,3,2-dioxaborolane-2-yl)benzoate obtained in Reference Example 225 were used in the same manner as in Reference Example 4 to obtain the titled compound. Yield: 47%.

¹H-NMR (CDCl₃) δ: 1.41 (3H, t, J=7.2 Hz), 1.89 (1H, br s), 2.44 (3H, s), 4.41 (2H, q, J=7.2 Hz), 4.93 (2H, s), 7.38 (1H, dd, J=7.2, 1.1 Hz), 7.50 (1H, t, J=7.8 Hz), 7.58-7.67 (1H, m), 7.71 (1H, dd, J=8.0, 1.1 Hz), 7.74-7.81 (1H, m), 8.17 (1H, t, J=1.5 Hz).

### Reference Example 336

### Ethyl 3-[2-(bromomethyl)-3-methyl-1-benzothiophen-7-yl]-5-fluorobenzoate

Phosphorus tribromide (287 μL, 3.05 mmol) was added dropwise while cooled on ice to a THF (15 mL) solution of ethyl

3-fluoro-5-[2-(hydroxymethyl)-3-methyl-1-benzothiophen-7-yl]benzoate (1.00 g, 2.90 mmol) obtained in Reference Example 335. The reaction solution was stirred for 1 hour at room temperature, was then poured into ice, and was extracted with ethyl acetate. The combined organic layers were washed with saturated sodium bicarbonate aqueous solution, dried over anhydrous magnesium sulfate, and then concentrated at reduced pressure to give 1.18 g of the titled compound (yield quantitative).

¹H-NMR (CDCl₃) δ: 1.41 (3H, t, J=7.2 Hz), 2.44 (3H, s), 4.42 (2H, q, J=7.2 Hz), 4.78 (2H, s), 7.41 (1H, d, J=7.2 Hz), 7.51 (1H, t, J=7.6 Hz), 7.61 (1H, dt, J=9.3, 2.2 Hz), 7.72 (1H, d, J=8.0 Hz), 7.74-7.82 (1H, m), 8.16 (1H, s).

### Reference Example 337

### Ethyl 3-fluoro-5-[3-methyl-2-[3-(trifluoromethyl)benzyl]-1-benzothiophen-7-yl]benzoate

Ethyl 3-[2-(bromomethyl)-3-methyl-1-benzothiophen-7-yl]-5-fluorobenzoate obtained in Reference Example 336, and 3-trifluoromethylphenylboronic acid were used in the same manner as in Reference Example 4 to obtain the titled compound. Yield: 52%.

¹H-NMR (CDCl₃) δ: 1.35-1.43 (3H, m), 2.42 (3H, s), 4.27 (2H, s), 4.39 (2H, q, J=7.2 Hz), 7.36 (1H, d, J=7.2 Hz), 7.38-7.43 (2H, m), 7.44-7.54 (3H, m), 7.55-7.63 (1H, m), 7.70 (1H, d, J=8.0 Hz), 7.74 (1H, dd, J=8.7, 1.5 Hz), 8.15 (1H, d, J=1.5 Hz).

### Reference Example 338

### 3-Fluoro-5-[3-methyl-2-[3-(trifluoromethyl)benzyl]-1-benzothiophen-7-yl]benzoic acid

Ethyl 3-fluoro-5-[3-methyl-2-[3-(trifluoromethyl)benzyl]-1-benzothiophen-7-yl]benzoate obtained in Reference Example 337 was used in the same manner as in Reference Example 5 to obtain the titled compound. Yield: 89% melting point: 168-169 ° C. (ethyl acetate).

¹H-NMR (DMSO-d₆) δ: 2.44 (3H, s), 4.39 (2H, s), 7.45-7.50 (1H, m), 7.52-7.61 (4H, m), 7.65 (1H, s), 7.68-7.78 (2H, m), 7.81 (1H, d, J=6.8 Hz), 8.08 (1H, s), 13.50 (1H, br s).

### Reference Example 339

### (7-Bromo-1-benzothiophen-2-yl)[3-(trifluoromethyl)phenyl]methanol

A THF (10 mL) solution of 7-bromo-1-benzothiophen-2-carbaldehyde (1.00 g, 4.15 mmol) was added dropwise in a nitrogen atmosphere while cooled on ice to a THF (10 mL) solution of Grignard reagent prepared from magnesium (202 mg, 8.30 mmol) and 1-bromo-3-(trifluoromethyl)benzene (1.16 mL, 8.30 mmol). The reaction solution was stirred for 2 hours at room temperature, was then poured into a mixture of ice and ammonium chloride, and was extracted with ethyl acetate. The combined organic layers were washed with water, dried over anhydrous magnesium sulfate, and then concentrated at reduced pressure. The residue was purified by silica gel column chromatography (hexane-ethyl acetate 100:0→85:15) to give 1.59 g of the titled compound (yield 99%).

¹H-NMR (CDCl₃) δ: 2.70 (1H, d, J=3.8 Hz), 6.17 (1H, d, J=2.7 Hz), 7.17-7.28 (2H, m), 7.45 (1H, d, J=7.6 Hz), 7.47-7.55 (1H, m), 7.56-7.62 (1H, m), 7.62-7.71 (2H, m), 7.78 (1H, s).

### Reference Example 340

### Ethyl 3-(2-[hydroxy[3-(trifluoromethyl)phenyl]methyl]-1-benzothiophen-7-yl)benzoate

(7-Bromo-1-benzothiophen-2-yl)[3-(trifluoromethyl)phenyl]methanol obtained in Reference Example 339, and [3-(ethoxycarbonyl)phenyl]boronic acid were used in the same manner as in Reference Example 4 to obtain the titled compound. Yield: 92%.

¹H-NMR (CDCl₃) δ: 1.38 (3H, t, J=7.2 Hz), 2.72 (1H, d, J=4.1 Hz), 4.39 (2H, q, J=7.2 Hz), 6.17 (1H, d, J=3.4 Hz), 7.24 (1H, d, J=0.8 Hz), 7.32-7.39 (1H, m), 7.42-7.51 (2H, m), 7.51-7.61 (2H, m), 7.66 (1H, d, J=7.5 Hz), 7.72 (1H, dd, J=7.9, 1.1 Hz), 7.77 (1H, s), 7.88 (1H, d, J=8.3 Hz), 8.07 (1H, d, J=7.9 Hz), 8.32 (1H, s).

### Reference Example 341

### 3-(2-[Hydroxy[3-(trifluoromethyl)phenyl]methyl]-1-benzothiophen-7-yl)benzoic acid

Ethyl 3-(2-[hydroxy[3-(trifluoromethyl)phenyl]methyl]-1-benzothiophen-7-yl)benzoate obtained in Reference Example 340 was used in the same manner as in Reference Example 5 to obtain the titled compound. Yield: 96%, amorphous solid.

¹H-NMR (CDCl₃) δ: 6.19 (1H, s), 7.25 (1H, s), 7.34-7.41 (1H, m), 7.43-7.53 (2H, m), 7.56-7.63 (2H, m), 7.67 (1H, d, J=7.6 Hz), 7.73 (1H, d, J=8.0 Hz), 7.78 (1H, s), 7.95 (1H, d, J=6.8 Hz), 8.15 (1H, d, J=8.0 Hz), 8.41 (1H, s), 2H unconfirmed.

### Reference Example 342

### 7-Bromo-N-methoxy-N-methyl-1-benzothiophene-2-carboxamide

Triethylamine (2.03 mL, 14.6 mmol) was added to a DMF (25 mL) solution of 7-bromo-1-benzothiophene-2-carboxylic acid (2.50 g, 9.72 mmol), N-methoxymethanamine hydrochloride (1.14 g, 11.7 mmol), WSC (2.24 g, 11.7 mmol), and HOBt (1.58 g, 11.7 mmol), and the mixture was stirred for 15 hours at room temperature. The addition of saturated sodium bicarbonate aqueous solution to the reaction solution was followed by extraction with ethyl acetate. The combined organic layers were washed with water, dried over anhydrous magnesium sulfate, and then concentrated at reduced pressure. The residue was purified by silica gel column chromatography (ethyl acetate-hexane 100:0 75:25) to give 2.83 g of the titled compound (yield 97%).

¹H-NMR (CDCl₃) δ: 3.44 (3H, s), 3.86 (3H, s), 7.24-7.33 (1H, m), 7.58 (1H, d, J=7.6 Hz), 7.84 (1H, d, J=8.0 Hz), 8.30 (1H, s).

### Reference Example 343

### (7-Bromo-1-benzothiophen-2-yl)[3-(trifluoromethyl )phenyl]methanone

A THF (30 mL) solution of 7-bromo-N-methoxy-N-methyl-1-benzothiophene-2-carboxamide (2.83 g, 9.43 mmol) obtained in Reference Example 342 was added dropwise while cooled on ice to a THF (30 mL) solution of Grignard reagent prepared from magnesium (343 mg, 14.1 mmol) and 1-bromo-3-(trifluoromethyl)benzene (1.97 mL, 14.1 mmol). The reaction solution was stirred for 3 hours at room temperature, was then poured into a mixture of ice and ammonium chloride, and was extracted with ethyl acetate. The combined organic layers were washed with water, dried over anhydrous magnesium sulfate, and then concentrated at reduced pressure to give 3.63 g of the titled compound (yield quantitative). Melting point: 135-136° C. (ethyl acetate).

¹H-NMR (CDCl₃) δ: 7.34 (1H, t, J=7.8 Hz), 7.61-7.76 (2H, m), 7.84-7.92 (2H, m), 7.93 (1H, s), 8.11 (1H, d, J=7.6 Hz), 8.17 (1H, s).

### Reference Example 344

### 1-(7-Bromo-1-benzothiophen-2-yl)-1-[3-(trifluoromethyl)phenyl]ethanol

A THF solution (1.0 mol/L, 7.78 mL, 7.78 mmol) of methyl magnesium bromide was added dropwise while cooled on ice to a THF (25 mL) solution of (7-bromo-1-benzothiophen-2-yl)[3-(trifluoromethyl)phenyl]methanone (1.50 g, 3.89 mmol) obtained in Reference Example 343, and the mixture was stirred for 1 hour at room temperature. The reaction solution was poured into a mixture of ice and ammonium chloride, and the mixture was extracted with ethyl acetate. The combined organic layers were washed with water, dried over anhydrous magnesium sulfate, and then concentrated at reduced pressure. The residue was purified by silica gel column chromatography (ethyl acetate-hexane 100:0→85:15) to give 1.56 g of the titled compound (yield quantitative) in the form of an oily substance.

¹H-NMR (CDCl₃) δ: 2.10 (3H, s), 2.60 (1H, s), 7.19-7.25 (1H, m), 7.25 (1H, s), 7.43-7.51 (2H, m), 7.53-7.60 (1H, m), 7.65 (1H, d, J=8.0 Hz), 7.69 (1H, d, J=7.6 Hz), 7.87 (1H, s).

### Reference Example 345

### Ethyl 3-(2-[1-hydroxy-1-[3-(trifluoromethyl)phenyl]ethyl]-1-benzothiophen-7-yl)benzoate

1-(7-Bromo-1-benzothiophen-2-yl)-1-[3-(trifluoromethyl)phenyl]ethanol obtained in Reference Example 344, and [3-(ethoxycarbonyl)phenyl]boronic acid were used in the same manner as in Reference Example 4 to obtain the titled compound. Yield Quantitative. Oily substance.

¹H-NMR (CDCl₃) δ: 1.38 (3H, t, J=7.0 Hz), 2.09 (3H, s), 2.60 (1H, s), 4.39 (2H, q, J=7.2 Hz), 7.25 (1H, s), 7.32-7.39 (1H, m), 7.40-7.49 (2H, m), 7.49-7.58 (2H, m), 7.68 (1H, d, J=8.0 Hz), 7.72 (1H, dd, J=8.0, 1.1 Hz), 7.83-7.93 (2H, m), 8.07 (1H, d, J=8.0 Hz), 8.31 (1H, s).

### Reference Example 346

### 3-(2-[1-Hydroxy-1-[3-(trifluoromethyl)phenyl]ethyl]-1-benzothiophen-7-yl)benzoic acid

Ethyl 3-(2-[1-hydroxy-1-[3-(trifluoromethyl)phenyl]ethyl]-1-benzothiophen-7-yl)benzoate obtained in Reference Example 345 was used in the same manner as in Reference Example 5 to obtain the titled compound. Yield: 94%, amorphous solid.

¹H-NMR (CDCl₃) δ: 2.10 (3H, s), 7.25 (1H, s), 7.35.-7.41 (1H, m), 7.41-7.51 (2H, m), 7.51-7.56 (1H, m), 7.59 (1H, t, J=7.8 Hz), 7.69 (1H, d, J=8.0 Hz), 7.74 (1H, d, J=6.8 Hz), 7.87 (1H, s), 7.96 (1H, d, J=7.6 Hz), 8.15 (1H, d, J=7.6 Hz), 8.40 (1H, s), 2H unconfirmed.

### Reference Example 347

### 7-Bromo-2-[1-[3-(trifluoromethyl)phenyl]ethyl]-1-benzothiophene

Triethylsilane (359 μL, 2.25 mmol) was added to a trifluoroacetic acid (2 mL) solution of 1-(7-bromo-1-benzothiophen-2-yl)-1-[3-(trifluoromethyl)phenyl]ethanol (300 mg, 0.75 mmol) obtained in Reference Example 344, and the mixture was stirred for 17 hours at room temperature. The reaction solution was concentrated at reduced pressure, was treated with saturated sodium bicarbonate aqueous solution, and was then extracted with ethyl acetate. The combined organic layers were washed with water, dried over anhydrous magnesium sulfate, and then concentrated at reduced pressure. The residue was purified by silica gel column chromatography (ethyl acetate-hexane 100:0→92:8) to give 200 mg of the titled compound (yield 69%) in the form of an oily substance.

¹H-NMR (CDCl₃) δ: 1.79 (3H, d, J=6.8 Hz), 4.47 (1H, q, J=6.9 Hz), 7.13 (1H, s), 7.16-7.24 (1H, m), 7.36-7.54 (4H, m), 7.56 (1H, s), 7.62 (1H, d, J=8.0 Hz).

### Reference Example 348

### Ethyl 3-(2-[1-[3-(trifluoromethyl)phenyl]ethyl]-1-benzothiophen-7-yl)benzoate

7-Bromo-2-[1-[3-(trifluoromethyl)phenyl]ethyl]-1-benzothiophene obtained in Reference Example 347, and [3-(ethoxycarbonyl)phenyl]boronic acid were used in the same manner as in Reference Example 4 to obtain the titled compound. Yield: 88%, oily substance.

¹H-NMR (CDCl₃) δ: 1.38 (3H, t, J=7.2 Hz), 1.78 (3H, d, J=7.2 Hz), 4.34-4.44 (2H, m), 4.41-4.51 (1H, m), 7.13 (1H, s), 7.28-7.36 (1H, m), 7.37-7.59 (6H, m), 7.70 (1H, d, J=6.8 Hz), 7.87 (1H, d, J=7.6 Hz), 8.07 (1H, d, J=8.0 Hz), 8.33 (1H, t, J=1.7 Hz).

### Reference Example 349

### 3-(2-[1-[3-(Trifluoromethyl)phenyl]ethyl]-1-benzothiophen-7-yl)benzoic acid

Ethyl 3-(2-[1-[3-(trifluoromethyl)phenyl]ethyl]-1-benzothiophen-7-yl)benzoate obtained in Reference Example 348 was used in the same manner as in Reference Example 5 to obtain the titled compound. Yield: 95%, amorphous solid.

¹H-NMR (CDCl₃) δ: 1.78 (3H, d, J=7.2 Hz), 4.46 (1H, q, J=6.8 Hz), 7.13 (1H, d, J=1.1 Hz), 7.31-7.37 (1H, m), 7.40-7.52 (4H, m), 7.55 (1H, s), 7.59 (1H, t, J=7.8 Hz), 7.71 (1H, d, J=8.0 Hz), 7.95 (1H, d, J=8.0 Hz), 8.14 (1H, d, J=8.0 Hz), 8.41 (1H, s), 1H unconfirmed.

### Reference Example 350

### Methyl [(2-bromo-4-fluorophenyl)sulfanyl]acetate

A DMSO mixture of 2-bromo-4-fluorobenzenethiol (5.0 g, 24.2 mmol), methyl bromoacetate (2.5 mL, 26.6 mmol), and pyridine (2.14 g, 26.6 mmol) was stirred for 2 hours. The reaction solution was diluted with a hexane-ethyl acetate (3:1, 120 mL) mixture, then washed with water, and dried over anhydrous sodium sulfate, and the solvent was then distilled off at reduced pressure. The residue was purified by silica gel column chromatography (hexane-ethyl acetate=85:15) to give 5.9 g of the titled compound (yield 87%) in the form of an oily substance.

¹H-NMR (CDCl₃) δ: 3.63 (2H, s), 3.71 (3H, s), 7.02 (1H, td, J=8.3, 2.7 Hz), 7.36 (1H, dd, J=8.0, 2.7 Hz), 7.48 (1H, dd, J=8.7, 6.1 Hz).

### Reference Example 351

### [(2-Bromo-4-fluorophenyl)sulfanyl]acetic acid

2 N sodium hydroxide aqueous solution (20 mL, 40 mmol) was added to a THF (100 mL) solution of methyl [(2-bromo-4-fluorophenyl)sulfanyl]acetate (5.58 g, 20 mmol) obtained in Reference Example 350, and the mixture was stirred for 2 hours at room temperature. The reaction solution was neutralized with the addition of 6 N hydrochloric acid, and was then extracted with ethyl acetate. The organic layer was washed with water, dried over anhydrous sodium sulfate, and then concentrated at reduced pressure to give 5.3 g of the titled compound (yield 100%) in the form of colorless solids.

¹H-NMR (CDCl₃) δ: 3.66 (2H, s), 7.02 (1H, td, J=8.3, 2.7 Hz), 7.37 (1H, dd, J=8.3, 2.7 Hz), 7.48 (1H, dd, J=8.7, 5.7 Hz); 8.81 (1H, br s).

### Reference Example 352

### 7-Bromo-5-fluoro-2-[3-(trifluoromethyl)benzyl]-1-benzothiophene

A chlorobenzene (5 mL) mixture of [(2-bromo-4-fluorophenyl)sulfanyl]acetic acid (530 mg, 2.0 mmol) obtained in Reference Example 351 and thionyl chloride (0.29 mL, 4.0 mmol) was stirred for 2 hours at 50° C. The reaction mixture was cooled to room temperature, aluminum chloride (798 mg, 6.0 mmol) was then added, and the mixture was stirred for 1 hour at room temperature. The reaction solution was poured into water, and the mixture was extracted with ethyl acetate. The organic layer was washed with saturated brine, dried over anhydrous sodium sulfate, and then concentrated at reduced pressure. The residue was dissolved in toluene (5 mL), 3-(trifluoromethyl)benzaldehyde (0.27 mL, 2.0 mmol) and piperidine (0.1 mL) were added, and the mixture was stirred over night at 100° C. The reaction solution was diluted with ethyl acetate, then washed with water, and dried over anhydrous sodium sulfate, and the solvent was then distilled off at reduced pressure. The residue was recrystallized using hexane-ethyl acetate to give yellow solids (409 mg). Triethylsilane (0.40 mL) and trifluoromethanesulfonic acid (0.22 mL) were added to a toluene (5 mL) solution of the resulting yellow solids (400 mg), and the mixture was stirred for 2 hours at room temperature. The reaction solution was diluted with ethyl acetate, then washed with sodium bicarbonate aqueous solution and saturated brine, dried over anhydrous sodium sulfate, and then concentrated at reduced pressure. The residue was purified by silica gel column chromatography (hexane-ethyl acetate=95:5) to give 42 mg of the titled compound (yield 5%) in the form of an oily substance.

¹H-NMR (CDCl₃) δ: 4.28 (2H, s), 7.08 (1H, t, J=1.1 Hz), 7.24 (1H, dd, J=8.2, 2.2 Hz), 7.31 (1H, dd, J=9.1, 2.2 Hz), 7.45-7.58 (4H, m).

### Reference Example 353

### Ethyl 3-[5-fluoro-2-[3-(trifluoromethyl)benzyl]-1-benzothiophen-7-yl]benzoate

A 1,2-dimethoxyethane (2 mL) mixture of 7-bromo-5-fluoro-2-[3-(trifluoromethyl)benzyl]-1-benzothiophene (190 mg, 0.49 mmol) obtained in Reference Example 352, (3-(ethoxycarbonyl)phenyl)boronic acid (142 mg, 0.73 mmol), tetrakis(triphenylphosphine)palladium (0) (28 mg, 0.024 mmol), and 2 N sodium carbonate aqueous solution (0.49 mL) was reacted for 2 hours at 80° C. in a nitrogen atmosphere. The reaction solution was diluted with ethyl acetate, then washed with saturated brine, dried over anhydrous sodium sulfate, and then concentrated at reduced pressure. The residue was purified by silica gel column chromatography (hexane-ethyl acetate=90:10) to give 222 mg of the titled compound (yield 99%) in the form of an oily substance.

¹H-NMR (CDCl₃) δ: 1.40 (3H, t, J=7.2 Hz), 4.26 (2H, s), 4.40 (2H, q, J=7.2 Hz), 7.05-7.08 (1H, m), 7.12 (1H, dd, J=9.5, 2.3 Hz), 7.36 (1H, dd, J=8.9, 2.5 Hz), 7.39-7.60 (5H, m), 7.81-7.89 (1H, m), 8.07-8.13 (1H, m), 8.31-8.35 (1H, m).

### Reference Example 354

### 3-[5-Fluoro-2-[3-(trifluoromethyl)benzyl]-1-benzothiophen-7-yl]benzoic acid

A THF (2 mL)-ethanol (1 mL) mixture of ethyl 3-[5-fluoro-2-[3-(trifluoromethyl)benzyl]-1-benzothiophen-7-yl]benzoate (220 mg, 0.48 mmol) obtained in Reference Example 353 and 1 N sodium hydroxide aqueous solution (1.4 mL) was stirred for 2 hours at 80° C. The reaction solution was cooled to room temperature, then neutralized with 1 N hydrochloric acid, and extracted with ethyl acetate. The organic layer was washed with saturated brine, dried over anhydrous sodium sulfate, and then concentrated at reduced pressure to give 200 mg of the titled compound (yield 97%) in the form of colorless solids.

¹H-NMR (CDCl₃) δ: 4.27 (2H, s), 7.06 (1H, t, J=0.9 Hz), 7.13 (1H, dd, J=9.6, 2.4 Hz), 7.37 (1H, dd, J=9.0, 2.3 Hz), 7.40-7.65 (5H, m), 7.88-7.95 (1H, m), 8.14-8.21 (1H, m), 8.40-8.43 (1H, m).

### Reference Example 355

### [4-(4,4,5,5-Tetramethyl-1,3,2-dioxaborolan-2-yl)-1-benzothiophen-2-yl]methanol

A DMF (12.7 mL) solution of (4-bromo-1-benzothiophen-2-yl)methanol (845 mg, 3.48 mmol) obtained in Reference Example 161, bispinacolatodiboron (971 mg, 3.82 mmol), 1,1-bis-(diphenylphosphino)-ferrocene palladium dichloride (41.9 mg, 0.174 mmol), and potassium acetate (1.02 g, 10.4 mmol) was stirred over night at 80° C. in a nitrogen atmosphere. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate:hexane=1:4) to give 858 mg of the titled compound (yield 85%) in the form of an oily substance.

¹H-NMR (CDCl₃) δ: 1.39 (12H, s), 1.96 (1H, t, J=6.2 Hz), 4.90-5.01 (2H, m), 7.24-7.37 (1H, m), 7.79-7.98 (3H, m)

### Reference Example 356

### Methyl 2-[2-(hydroxymethyl)-1-benzothiophen-4-yl]pyridine-4-carboxylate

[4-(4,4,5,5-Tetramethyl-1,3,2-dioxaborolan-2-yl)-1-benzothiophen-2-yl]methanol obtained in Reference Example 355 and methyl 2-bromopyridine-4-carboxylate were used in the same manner as in Reference Example 4 to obtain the titled compound in the form of an oily substance. Yield: 40%.

¹H-NMR (CDCl₃) δ: 2.14-2.23 (1H, m), 4.00 (3H, s), 4.94 (2H, d, J=5.7 Hz), 7.38-7.47 (1H, m), 7.59-7.67 (1H, m), 7.74 (1H, s), 7.80-7.87 (1H, m), 7.90 (1H, d, J=7.9 Hz), 8.24 (1H, s), 8.90 (1H, d, J=6.0 Hz)

### Reference Example 357

### Methyl 2-[2-(bromomethyl)-1-benzothiophen-4-yl]pyridine-4-carboxylate

Phosphorus tribromide (0.138 mL, 1.46 mmol) was added dropwise while cooled on ice to a THF solution (6.3 mL) of methyl 2-[2-(hydroxymethyl)-1-benzothiophen-4-yl]pyridine-4-carboxylate (418 mmol, 1.40 mmol) obtained in Reference Example 356, and the mixture was stirred for 1 hour at 0° C. The reaction solution was concentrated, the residue was dissolved in ethyl acetate, washed with saturated sodium bicoarbonate aqueous solution, and dried over anhydrous magnesium sulfate, and the solvent was distilled off at reduced pressure. The residue was purified by silica gel column chromatography (ethyl acetate:hexane=1:4) to give 253 mg of the titled compound (yield 50%) in the form of an oily substance.

¹H-NMR (CDCl₃) δ: 4.01 (3H, s), 4.81 (2H, s), 7.42-7.50 (1H, m), 7.62-7.69 (1H, m), 7.83-7.93 (3H, m), 8.22-8.26 (1H, m), 8.90-8.95 (1H, m)

### Reference Example 358

### Methyl 2-[2-[3-(trifluoromethyl)benzyl]-1-benzothiophen-4-yl]pyridine-4-carboxylate

A 2 N sodium carbonate aqueous solution (1.0 mL)-1,2-dimethoxyethane (5.0 mL) mixture of methyl 2-[2-(bromomethyl)-1-benzothiophen-4-yl]pyridine-4-carboxylate (250 mg, 0.690 mmol) obtained in Reference Example 357, 3-trifluororniethylphenylboronic acid (157 mg, 0.828 mmol), and tetrakis(triphenylphosphine)palladium (0) (95.7 mg, 0.083 mmol) was stirred for 2 hours at 95° C. in a nitrogen atmosphere. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1:4) to give 62.8 mg of the titled compound (yield 21%) in the form of an oily substance.

¹H-NMR (CDCl₃) δ: 3.99 (3H, s), 4.31 (2H, s), 7.34-7.54 (4H, m), 7.56 (1H, s), 7.59-7.66 (2H, m), 7.78-7.88 (2H, m), 8.23 (1H, s), 8.90 (1H, d, J=6.0 Hz).

### Reference Example 359

### 2-[2-[3-(Trifluoromethyl)benzyl]-1-benzothiophen-4-yl]pyridine-4-carboxylic acid

1 N sodium hydroxide aqueous solution (0.220 mL) was added at room temperature to a THF (1.0 mL)-methanol (0.5 mL) mixture of methyl 2-[2-[3-(trifluoromethyl)benzyl]-1-benzothiophen-4-yl]pyridine-4-carboxylate (62.8 mg, 0.147 mmol) obtained in Reference Example 358, and the mixture was stirred over night at room temperature. Water was poured into the reaction solution, the pH was adjusted to between 2 and 3 with 1 N hydrochloric acid aqueous solution, and the mixture was extracted with ethyl acetate. The extract was washed with water and dried over anhydrous magnesium sulfate, and the solvent was distilled off at reduced pressure. Diethyl ether and hexane were added to the residue, and 48.5 mg of the titled compound (yield 80%) was filtered off in solid form.

¹H-NMR (DMSO-d₆) δ: 4.43 (2H, s), 7.44 (1H, t, J=7.9 Hz), 7.52-7.77 (6H, m), 7.84 (1H, dd, J=4.9, 1.5 Hz), 7.99 (1H, d, J=7.9 Hz), 8.14 (1H, s), 8.92 (1H, d, J=4.9 Hz),13.80 (1H, br s)

### Reference Example 360

### Ethyl 5-[2-[3-(trifluoromethyl)benzyl]-1-benzothiophen-7-yl]pyridine-3-carboxylate

4,4,5,5-Tetramethyl-2-[2-[3-(trifluoromethyl)benzyl]-1-benzothiophene-7-yl]-1,3,2-dioxaborolane obtained in Reference Example 193, and ethyl 5-bromopyridine-3-carboxylate were used in the same manner as in Reference Example 220 to obtain the titled compound in the form of an oily substance. Yield: 68%

¹H-NMR (CDCl₃) δ: 1.42 (3H, t, J=7.2 Hz), 4.28 (2H, s), 4.40-4.49 (2H, m), 7.13 (1H, s), 7.28-7.36 (1H, m), 7.38-7.57 (5H, m), 7.70-7.79 (1H, m), 8.59 (1H, t, J=2.1 Hz), 9.07 (1H, d, J=2.3 Hz), 9.24 (1H, d, J=1.9 Hz).

### Reference Example 361

### 5-[2-[3-(Trifluoromethyl)benzyl]-1-benzothiophen-7-yl]pyridine-3-carboxylic acid

Ethyl 5-[2-[3-(trifluoromethyl)benzyl]-1-benzothiophen-7-yl]pyridine-3-carboxylate obtained in Reference Example 360 was used in the same manner as in Reference Example 359 to obtain the titled compound in solid form. Yield: 76%

¹H-NMR (DMSO-d₆) δ: 4.41 (2H, s), 7.39 (1H, s), 7.45-7.69 (5H, m), 7.72 (1H, s), 7.85-7.94 (1H, m), 8.47-8.55 (1H, m), 9.02-9.20 (2H, m), 13.64 (1H, br s).

### Reference Example 362

### 4-[2-(3-Chloro-5-fluorobenzyl)-1-benzothiophen-7-yl]pyridine-2-carboxylic acid

A 2 N sodium carbonate aqueous solution (1.0 mL)-1,2-dimethoxyethane (5.5 mL) mixture of 2-[2-(3-chloro-5-fluorobenzyl)-1-benzothiophen-7-yl]-4,4,5,5-tetramethyl-1,3,2-dioxaborolane (275 mg, 0.683 mmol) obtained in Reference Example 192, methyl 4-bromopyridine-2-carboxylate (177 mg, 0.819 mmol), and tetrakis(triphenylphosphine)palladium (0) (94.7 mg, 0.082 mmol) was stirred for 4 hours at 95° C. in a nitrogen atmosphere. Water was poured into the reaction solution, and the mixture was extracted with ethyl acetate. Ethyl acetate was added to the residue, and 168 mg of the titled compound (yield 62%) was filtered off in solid form.

¹H-NMR (DMSO-d₆) δ: 4.31 (2H, s), 7.17-7.26 (1H, m), 7.26-7.33 (2H, m), 7.39 (1H, s), 7.43-7.58 (2H, m), 7.63 (1H, dd, J=4.9, 1.9 Hz), 7.87 (1H, dd, J=7.6, 1.9 Hz), 8.28 (1H, s), 8.63 (1H, d, J=4.9 Hz).

### Reference Example 363

### 2-Chloro-3-fluoro-N-(2-hydroxyethyl)pyridine-4-carboxamide

2-Chloro-3-fluoropyridine-4-carboxylic acid, and 2-aminoethanol were used in the same manner as in Reference Example 178 to obtain the titled compound in the form of an oily substance. Yield: 53%

¹H-NMR (CDCl₃) δ: 3.62-3.73 (2H, m), 3.82-3.90 (2H, m), 7.16 (1H, br s), 7.83 (1H, t, J=4.9 Hz), 8.33 (1H, d, J=4.9 Hz).

### Reference Example 364

### 2-Chloro-4-[2-[3-(trifluoromethyl)benzyl]-1-benzothiophen-7-yl]pyrimidine

4,4,5,5-Tetramethyl-2-[2-[3-(trifluoromethyl)benzyl]-1-benzothiophene-7-yl]-1,3,2-dioxaborolane obtained in Reference Example 193, and 2,4-dichloropyrimidine were used in the same manner as in Reference Example 4 to obtain the titled compound in solid form. Yield: 68%

¹H-NMR (DMSO-d₆) δ: 4.36 (2H, s), 7.08 (1H, s), 7.41-7.57 (4H, m), 7.60 (1H, s), 7.84 (1H, d, J=5.3 Hz), 7.88 (1H, d, J=8.0 Hz), 7.94 (1H, d, J=8.0 Hz), 8.69 (1H, d, J=5.3 Hz).

### Reference Example 365

### 4-[2-[3-(Trifluoromethyl)benzyl]-1-benzothiophen-7-yl]pyrimidine-2-carbonitrile

2-Chloro-4-[2-[3-(trifluoromethyl)benzyl]-1-benzothiophen-7-yl]pyrimidine (350 mg, 0.865 mmol) obtained in Reference Example 364 was added at room temperature to a DMF (3.5 mL) solution of sodium cyanide (63.6 mg, 1.30 mmol), and the mixture was stirred over night. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1:4) to give 90.2 mg of the titled compound (yield 26%) in solid form.

¹H-NMR (CDCl₃) δ: 4.35 (2H, s), 7.10 (1H, s), 7.41-7.62 (5H, m), 7.86-8.01 (2H, m), 8.08 (1H, d, J=5.7 Hz), 8.88 (1H, d, J=5.7 Hz).

### Reference Example 366

### 4-[2-[3-(Trifluoromethyl)benzyl]-1-benzothiophen-7-yl]pyrimidine-2-carboxylic acid

A mixture of 4-[2-[3-(trifluoromethyl)benzyl]-1-benzothiophen-7-yl]pyrimidine-2-carbonitrile (90 mg, 0.228 mmol) obtained in Reference Example 365 and 2 N sodium hydroxide aqueous solution (1.1 mL) was stirred for 6 hours at 120° C. Water was poured into the reaction solution, the pH was adjusted to between 2 and 3 with 1 N hydrochloric acid aqueous solution, and the mixture was extracted with ethyl acetate. The extract was washed with water and dried over anhydrous magnesium sulfate, and the solvent was distilled off at reduced pressure to give 90.0 mg of the titled compound (yield 95%) in solid form.

¹H-NMR (DMSO-d₆) δ: 4.43 (2H, s), 7.29 (1H, s), 7.48-7.69 (4H, m), 7.72 (1H, s), 7.98-8.07 (1H, m), 8.29 (1H, d, J=8.0 Hz), 8.46 (1H, d, J=5.7 Hz), 9.05 (1H, d, J=5.7 Hz).

### Reference Example 367

### 2-Bromo-5-fluoro-N-(2-hydroxyethyl)pyridine-4-carboxamide

2-Bromo-5-fluoropyridine-4-carboxylic acid, and 2-aminoethanol were used in the same manner as in Reference Example 178 to obtain the titled compound in solid form. Yield: 61%

¹H-NMR (CDCl₃) δ: 2.10 (1H, br s), 3.56-3.72 (2H, m), 3.79-3.90 (2H, m), 7.10 (1H, br s), 8.09 (1H, d, J=5.3 Hz), 8.37 (1H, d, J=2.3 Hz).

### Reference Example 368

### 7-Chloro-2-[3-(trifluoromethyl)phenoxy]-1,3-benzothiazole

A mixture of 2,7-dichloro-1,3-benzothiazole (1.0 g, 4.90 mmol), 3-trifluoromethylphenylboronic acid (953 mg, 5.88 mmol), Tris(dibenzylideneacetone)dipalladium (0) (89.7 mg, 0.098 mmol), and 2-(dicyclohexylphosphino)-2′,4′,6′-triisopropyl-1,1′-biphenyl (140 mg, 0.294 mmol), and cesium carbonate (2.39 g, 7.35 mmol) in toluene (20 mL) was stirred for 5 hours at 100° C. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1:4) to give 1.52 g of the titled compound (yield 94%) in solid form.

¹H-NMR (CDCl₃) δ: 7.27-7.40 (2H, m), 7.54-7.70 (5H, m).

### Reference Example 369

### Ethyl 3-[2-[3-(trifluoromethyl)phenoxy]-1,3-benzothiazol-7-yl]benzoate

A THF (10 mL) solution of 7-chloro-2-[3-(trifluoromethyl)phenoxy]-1,3-benzothiazole (500 mg, 1.52 mmol) obtained in Reference Example 368, [3-(ethoxycarbonyl)phenyl]boronic acid (353 mg, 1.82 mmol), palladium acetate (10.2 mg, 0.045 mmol), 2-(dicyclohexylphosphino)-2′,4′,6′-triisopropyl-1,1′-biphenyl (43.3 mg, 0.091 mmol), and potassium phosphate (644 mg, 3.03 mmol) was stirred for 5 hours at 60° C. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1:4) to give 484 mg of the titled compound (yield 72%) in solid form.

¹H-NMR (CDCl₃) δ: 1.38-1.44 (3H, m), 4.42 (2H, q, J=7.2 Hz), 7.39 (1H, d, J=6.4 Hz), 7.48-7.64 (5H, m), 7.67 (1H, s), 7.73-7.78 (1H, m), 7.80-7.86 (1H, m), 8.08-8.13 (1H, m), 8.30-8.34 (1H, m).

### Reference Example 370

### 3-[2-[3-(Trifluoromethyl)phenoxy]-1,3-benzothiazol-7-yl]benzoic acid

Ethyl 3-[2-[3-(trifluoromethyl)phenoxy]-1,3-benzothiazol-7-yl]benzoate obtained in Reference Example 369 was used in the same manner as in Reference Example 359 to obtain the titled compound in solid form. Yield: 74%

¹H-NMR (DMSO-d₆) δ: 7.49-7.56 (1H, m), 7.56-7.64 (1H, m), 7.65-7.73 (1H, m), 7.74-7.82 (3H, m), 7.82-7.88 (1H, m), 7.89-7.98 (2H, m), 7.99-8.08 (1H, m), 8.20-8.24 (1H, m), 13.20 (1H, br s).

### Reference Example 371

### N-(2-Amino-2-oxoethyl)-2-chloro-3-fluoropyridine-4-carboxamide

A DMF (6.0 mL) solution of 2-chloro-3-fluoropyridine-4-carboxylic acid (300 mg, 1.71 mmol), WSC (393 mg, 2.05 mmol), HOBt (137 mg, 2.05 mmol), glycinamide hydrochloride (208 mg, 1.87 mL), and N,N-diisopropylethylamine (0.327 mL, 1.87 mmol) was stirred for 3 hours at room temperature. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate) and recrystallized from ethyl acetate and hexane to give 71.5 mg of the titled compound (yield 18%) in solid form.

¹H-NMR (CDCl₃) δ: 4.20 (2H, d, J=4.9 Hz), 5.54 (1H, br s), 5.72 (1H, br s), 7.41 (1H, br s), 7.84 (1H, t, J=4.9 Hz), 8.35 (1H, d, J=4.9 Hz)

### Reference Example 372

### 7-Bromo-2-[3-(trifluoromethyl)benzyl]-1,3-benzothiazole

Oxalyl chloride (3.45 ml, 40.8 mmol) was added dropwise at room temperature to a THF (170 mL)-DMF (1 mL) solution of [3-(trifluoromethyl)phenyl]acetic acid (7.63 g, 37.4 mmol), the mixture was stirred for 1 hour, and the reaction solution was concentrated. The residue was dissolved in THF (170 mL). 2,3-dibromoaniline (8.53 g, 34.0 mmol), N,N-diisopropylethylamine (13.0 mL, 74.8 mmol), and 4-dimethylaminopyridine (415 mg, 3.40 mmol) were added, and the mixture was stirred for 3 hours at room temperature. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1:4) to give 14.62 g of N-(2,3-dibromophenyl)-2-[3-(trifluoromethyl)phenyl]acetamide (yield 98%) in solid form. Lawesson's reagent (3.05 g, 7.55 mmol) was added at room temperature to a toluene (60 mL) solution of the resulting N-(2,3-dibromophenyl)-2-[3-(trifluoromethyl)phenyl]acetamide (3.0 g, 6.86 mmol), and the mixture was stirred for 2 hours at 100° C. Water was poured into the reaction solution, and the mixture was extracted with ethyl acetate. The extract was dried over anhydrous magnesium sulfate, and the solvent was distilled off at reduced pressure to give 2.59 g of N-(2,3-dibromophenyl)-2-[3-(trifluoromethyl)phenyl]ethanamide (yield 83%) in the form of an oily substance. A toluene (50 mL) solution of the resulting N-(2,3-dibromophenyl)-2-[3-(trifluoromethyl)phenyl]ethanamide (2.59 g, 5.72 mmol), Tris(dibenzylideneacetone)dipalladium (0) (523 mg, 0.572 mmol), biphenyl-2-yl(di-tert-butyl)phosphine (188 mg, 0.629 mmol), and cesium carbonate (2.79 g, 8.57 mmol) was stirred for 6 hours at 100° C. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1:19) to give 220 mg of the titled compound (yield 10%) in the form of an oily substance.

¹H-NMR (CDCl₃) δ: 4.49 (2H, s), 7.32-7.40 (1H, m), 7.46-7.53 (2H, m), 7.54-7.60 (2H, m), 7.64 (1H, br s), 7.91-7.97 (1H, m).

### Reference Example 373

### Ethyl 3-[2-[3-(trifluoromethyl)benzyl]-1,3-benzothiazol-7-yl]benzoate

7-Bromo-2-[3-(trifluoromethyl)benzyl]-1,3-benzothiazole obtained in Reference Example 372, and [3-(ethoxycarbonyl)phenyl]boronic acid were used in the same manner as in Reference Example 4 to obtain the titled compound in solid form. Yield: 49%

¹H-NMR (CDCl₃) δ: 1.39 (3H, t, J=7.0 Hz), 4.35-4.44 (2H, m), 4.50 (2H, s), 7.42-7.50 (2H, m), 7.50-7.67 (5H, m), 7.77-7.84 (1H, m), 7.98-8.11 (2H, m), 8.27-8.33 (1H, m).

### Reference Example 374

### 3-[2-[3-(Trifluoromethyl)benzyl]-1,3-benzothiazol-7-yl]benzoic acid

Ethyl 3-[2-[3-(trifluoromethyl)benzyl]-1,3-benzothiazol-7-yl]benzoate obtained in Reference Example 373 was used in the same manner as in Reference Example 5 to obtain the titled compound in solid form. Yield: 99%

¹H-NMR (DMSO-d₆) δ: 4.65 (2H, s), 7.54-7.77 (6H, m), 7.82 (1H, br s), 7.89-7.95 (1H, m), 7.99-8.07 (2H, m), 8.19-8 24 (1H, m), 13.20 (1H, br s).

### Reference Example 375

### 3-Bromo-2-fluoro-N-(2-methoxyethyl)benzamide

3-Bromo-2-fluorobenzoic acid, and 2-methoxyethylamine were used in the same manner as in Reference Example 178 to obtain the titled compound in solid form. Yield: 69%

¹H-NMR (CDCl₃) δ: 3.40 (3H, s), 3.54-3.61 (2H, m), 3.64-3.72 (2H, m), 7.00 (1H, br s), 7.14 (1H, t, J=7.9 Hz), 7.62-7.71 (1H, m), 7.96-8.04 (1H, m).

### Reference Example 376

### N-(2-amino-2-oxoethyl)-3-bromo-2-fluorobenzamide

3-Bromo-2-fluorobenzoic acid, and glycinamide hydrochloride were used in the same manner as in Reference Example 371 to obtain the titled compound in solid form. Yield: 70%

¹H-NMR (CDCl₃) δ: 4.19 (2H, d, J=3.8 Hz), 5.44 (1H, br s), 5.83 (1H, br s), 7.17 (1H, t, J=8.3 Hz), 7.33 (1H, br s), 7.67-7.76 (1H, m), 7.95-8.05 (1H, m).

### Reference Example 377

### 2-Chloro-3-fluoro-N-(2-methoxyethyl)pyridine-4-carboxamide

2-Chloro-3-fluoropyridine-4-carboxylic acid, and 2-methoxyethylamine were used in the same manner as in Reference Example 178 to obtain the titled compound in solid form. Yield: 62%

¹H-NMR (CDCl₃) δ: 3.41 (3H, s), 3.54-3.61 (2H, m), 3.64-3.73 (2H, m), 7.01 (1H, br s), 7.85 (1H, t, J=4.9 Hz), 8.33 (1H, d, J=4.9 Hz).

### Reference Example 378

### Ethyl 3-[2-(3-chloro-5-fluorobenzyl)-1-benzothiophen-7-yl]-4-fluorobenzoate

2-[2-(3-Chloro-5-fluorobenzyl)-1-benzothiophene-7-yl]-4,4,5,5-tetramethyl-1,3,2-dioxaborolane obtained in Reference Example 192, and ethyl 3-bromo-4-fluorobenzoate were used in the same manner as in Reference Example 220 to obtain the titled compound in the form of an oily substance. Yield: 60%

¹H-NMR (CDCl₃) δ: 1.37 (3H, t, J=7.2 Hz), 4.17 (2H, s), 4.38 (2H, q, J=7.2 Hz), 6.83-6.92 (1H, m), 6.93-7.00 (1H, m), 7.06 (1H, s), 7.12 (1H, s), 7.20-7.34 (2H, m), 7.38-7.48 (1H, m), 7.69-7.78 (1H, m), 8.06-8.15 (1H, m), 8.28 (1H, dd, J=7.2, 2.3 Hz).

### Reference Example 379

### 3-[2-(3-Chloro-5-fluorobenzyl)-1-benzothiophen-7-yl]-4-fluorobenzoic acid

Ethyl 3-[2-(3-chloro-5-fluorobenzyl)-1-benzothiophen-7-yl]-4-fluorobenzoate obtained in Reference Example 378 was used in the same manner as in Reference Example 359 to obtain the titled compound in solid form. Yield: 74%

¹H-NMR (DMSO-d₆) δ: 4.29 (2H, s), 7.17-7.25 (1H, m), 7.25-7.42 (4H, m), 7.44-7.58 (2H, m), 7.83-7.91 (1H, m), 8.02-8.15 (2H, m), 13.19 (1H, br s).

### Reference Example 380

### Methyl 4-methyl-3-[2-[3-(trifluoromethyl)benzyl]-1-benzothiophen-7-yl]benzoate

4,4,5,5-tetramethyl-2-[2-[3-(trifluoromethyl)benzyl]-1-benzothiophene-7-yl]-1,3,2-dioxaborolane obtained in Reference Example 193, and methyl 3-bromo-4-methylbenzoate were used in the same manner as in Reference Example 220 to obtain the titled compound in the form of an oily substance. Yield: 55%

¹H-NMR (CDCl₃) δ: 2.20 (3H, s), 3.88 (3H, s), 4.23 (2H, s), 7.07-7.15 (2H, m), 7.35-7.46 (4H, m), 7.47-7.53 (2H, m), 7.66-7.72 (1H, m), 7.95-8.02 (2H, m).

### Reference Example 381

### 4-Methyl-3-[2-[3-(trifluoromethyl)benzyl]-1-benzothiophen-7-yl]benzoic acid

Methyl 4-methyl-3-[2-[3-(trifluoromethyl)benzyl]-1-benzothiophen-7-yl]benzoate obtained in Reference Example 380 was used in the same manner as in Reference Example 359 to obtain the titled compound in solid form. Yield: 60%

¹H-NMR (DMSO-d₆) δ: 4.35 (2H, s), 7.19 (1H, d, J=6.4 Hz), 7.33 (1H, s), 7.40-7.66 (5H, m), 7.69 (1H, s), 7.77-7.84 (2H, m), 7.91 (1H, dd, J=7.9, 1.9 Hz), 12.92 (1H, s).

### Reference Example 382

### Ethyl 2-methyl-5-[2-[3-(trifluoromethyl)benzyl]-1-benzothiophen-7-yl]benzoate

4,4,5,5-tetramethyl-2-[2-[3-(trifluoromethyl)benzyl]-1-benzothiophene-7-yl]-1,3,2-dioxaborolane obtained in Reference Example 193, and ethyl 5-bromo-2-methylbenzoate were used in the same manner as in Reference Example 220 to obtain the titled compound in the form of an oily substance. Yield: 94%

¹H-NMR (CDCl₃) δ: 1.36 (3H, t, J=7.2 Hz), 2.65 (3H, s), 4.27 (2H, s), 4.36 (2H, q, J=7.2 Hz), 7.11 (1H, s), 7.28-7.37 (2H, m), 7.38-7.56 (5H, m), 7.65-7.74 (2H, m), 8.22 (1H, d, J=1.9 Hz).

### Reference Example 383

### Ethyl 6-fluoro-7-methoxy-1-benzothiophene-2-carboxylate

2,4-difluoro-3-methoxybenzaldehyde was used in the same manner as in Reference Example 154 to obtain the titled compound in the form of an oily substance. Yield: 28%

¹H-NMR (CDCl₃) δ: 1.41 (3H, t, J=7.2 Hz), 4.15 (3H, d, J=2.3 Hz), 4.40 (2H, q, J=7.2 Hz), 7.13-7.23 (1H, m), 7.49 (1H, dd, J=8.9, 4.0 Hz), 8.00 (1H, s).

### Reference Example 384

### 6-Fluoro-7-methoxy-1-benzothiophene-2-carboxylic acid

Ethyl 6-fluoro-7-methoxy-1-benzothiophene-2-carboxylate obtained in Reference Example 383 was used in the same manner as in Reference Example 158 to obtain the titled compound in solid form. Yield: 65%

¹H-NMR (DMSO-d₆) δ: 4.08 (3H, d, J=2.3 Hz), 7.37-7.48 (1H, m), 7.73 (1H, dd, J=8.7, 4.2 Hz), 8.11 (1H, s), 13.59 (1H, br s).

### Reference Example 385

### (6-Fluoro-7-methoxy-1-benzothiophen-2-yl)methanol

6-Fluoro-7-methoxy-1-benzothiophene-2-carboxylic acid obtained in Reference Example 384 was used in the same manner as in Reference Example 161 to obtain the titled compound in solid form. Yield: 75%

¹H-NMR (CDCl₃) δ: 1.90 (1H, t, J=6.1 Hz), 4.12 (3H, d, J=2.3 Hz), 4.90 (2H, d, J=6.1 Hz), 7.06-7.19 (2H, m), 7.34 (1H, dd, J=8.7, 4.2 Hz).

### Reference Example 386

### 2-(Bromomethyl)-6-fluoro-7-methoxy-1-benzothiophene

(6-Fluoro-7-methoxy-1-benzothiophen-2-yl)methanol obtained in Reference Example 385 was used in the same manner as in Reference Example 179 to obtain the titled compound in solid form. Yield: 50%

¹H-NMR (CDCl₃) δ: 4.12 (3H, d, J=2.3 Hz), 4.75 (2H, s), 7.07-7.28 (2H, m), 7.33 (1H, dd, J=8.7, 4.2 Hz)

### Reference Example 387

### 6-Fluoro-7-methoxy-2-[3-(trifluoromethyl)benzyl]-1-benzothiophene

A 2 N sodium carbonate aqueous solution (1.5 mL)-1,2-dimethoxyethane (4.0 mL) mixture of 2-(bromomethyl)-6-fluoro-7-methoxy-1-benzothiophene (200 mg, 0.727 mmol) obtained in Reference Example 386, 3-trifluoromethylphenylboronic acid (152 mg, 0.800 mmol), and tetrakis(triphenylphosphine)palladium (0) (42.0 mg, 0.036 mmol) was stirred over night at 95° C. in a nitrogen atmosphere. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1:9) to give 123 mg of the titled compound (yield 50%) in the form of an oily substance.

¹H-NMR (CDCl₃) δ: 4.09 (3H, d, J=2.3 Hz), 4.24 (2H, s), 6.96 (1H, s), 7.05-7.14 (1H, m), 7.23-7.32 (1H, m), 7.40-7.50 (2H, m), 7.50-7.58 (2H, m)

### Reference Example 388

### 6-Fluoro-2-[3-(trifluoromethyl)benzyl]-1-benzothiophen-7-ol

1.0 M boron tribromide-dichloromethane solution (3.53 mL, 3.53 mmol) was added dropwise while cooled on ice to a toluene (6 mL) solution of 6-fluoro-7-methoxy-2-[3-(trifluoromethyl)benzyl]-1-benzothiophene (300 mg, 0.881 mmol) obtained in Reference Example 387, and the mixture was stirred for 4 hours at room temperature. Water was poured into the reaction solution, and the mixture was extracted with ethyl acetate. The extract was dried over anhydrous magnesium sulfate, and the solvent was distilled off at reduced pressure to give the titled compound in solid form. The resulting solid was used without further modification in the following reaction.

### Reference Example 389

### 6-Fluoro-2-[3-(trifluoromethyl)benzyl]-1-benzothiophen-7-yl trifluoromethanesulfonate

Trifluoromethanesulfonic anhydride (0.297 mL, 1.76 mmol) was added dropwise while cooled on ice to a pyridine (1.4 mL) solution of 6-fluoro-2-[3-(trifluoromethyl)benzyl]-1-benzothiophen-7-ol (288 mg, 0.881 mmol) obtained in Reference Example 388, and the mixture was stirred for 1 hour at 0° C. Water was poured into the reaction solution, and the mixture was neutralized using 8 N sodium hydroxide aqueous solution and was extracted with ethyl acetate. The extract was dried over anhydrous magnesium sulfate, and the solvent was distilled off at reduced pressure. The residue was purified by silica gel column chromatography (ethyl acetate:hexane=1:1) to give 189 mg of the titled compound (yield 47%) in the form of an oily substance.

¹H-NMR (CDCl₃) δ: 4.28 (2H, s), 7.00 (1H, s), 7.22-7.30 (1H, m), 7.43-7.52 (2H, m), 7.52-7.67 (3H, m)

### Reference Example 390

### Ethyl 3-[6-fluoro-2-[3-(trifluoromethyl)benzyl]-1-benzothiophen-7-yl]benzoate

6-Fluoro-2-[3-(trifluoromethyl)benzyl]-1-benzothiophen-7-yl trifluoromethanesulfonate obtained in Reference Example 389, and [3-(ethoxycarbonyl)phenyl]boronic acid were used in the same manner as in Reference Example 4 to obtain the titled compound in the form of an oily substance. Yield: 26%

¹H-NMR (CDCl₃) δ: 1.38 (3H, t, J=7.2 Hz), 4.22 (2H, s), 4.34-4.44 (2H, m), 7.04 (1H, s), 7.16-7.23 (1H, m), 7.40-7.65 (6H, m), 7.73-7.80 (1H, m), 8.06-8.14 (1H, m), 8.26-8.32 (1H, m)

### Reference Example 391

### 3-[6-Fluoro-2-[3-(trifluoromethyl)benzyl]-1-benzothiophen-7-yl]benzoic acid

Ethyl 3-[6-fluoro-2-[3-(trifluoromethyl)benzyl]-1-benzothiophen-7-yl]benzoate obtained in Reference Example 390 were used in the same manner as in Reference Example 359 to obtain the titled compound in solid form. Yield: 78%

¹H-NMR (DMSO-d₆) δ: 4.35 (2H, s), 7.30-7.44 (2H, m), 7.50-7.73 (5H, m), 7.79-7.89 (2H, m), 8.01-8.08 (1H, m), 8.10 (1H, s), 13.16 (1H, s)

### Reference Example 392

### Methyl 2-methyl-6-[2-[3-(trifluoromethyl)benzyl]-1-benzothiophen-7-yl]pyridine-4-carboxylate

4,4,5,5-Tetramethyl-2-[2-[3-(trifluoromethyl)benzyl]-1-benzothiophene-7-yl]-1,3,2-dioxaborolane obtained in Reference Example 193, and methyl 2-chloro-6-methylpyridine-4-carboxylate were used in the same manner as in Reference Example 220 to obtain the titled compound in the form of an oily substance. Yield: 88%

¹H-NMR (CDCl₃) δ: 2.76 (3H, s), 3.98 (3H, s), 4.32 (2H, s), 7.07 (1H, s), 7.39-7.54 (4H, m), 7.61 (1H, s), 7.66 (1H, s), 7.76 (1H, dd, J=7.9, 1.1 Hz), 7.92 (1H, dd, J=7.9, 1.1 Hz), 8.31 (1H, s)

### Reference Example 393

### 2-Methyl-6-[2-[3-(trifluoromethyl)benzyl]-1-benzothiophen-7-yl]pyridine-4-carboxylic acid

Methyl 2-methyl-6-[2-[3-(trifluoromethyl)benzyl]-1-benzothiophen-7-yl]pyridine-4-carboxylate obtained in Reference Example 392 was used in the same manner as in Reference Example 359 to obtain the titled compound in solid form. Yield: 74%

¹H-NMR (DMSO-d₆) δ: 2.69 (3H, s), 4.41 (2H, s), 7.27 (1H, s), 7.45-7.71 (5H, m), 7.75 (1H, s), 7.89 (1H, d, J=7.2 Hz), 8.11 (1H, d, J=7.2 Hz), 8.34 (1H, s), 13.65 (1H, br s)

### Reference Example 394

### 4-Chlorothieno[3,2-d]-pyrimidine-6-carbaldehyde

n-Butyllithium (19.7 mL, 49.2 mmol, 2.5 M hexane solution) was added dropwise at −78° C. to an anhydrous THF (137 mL) solution of 4-chlorothieno[3,2-d]pyrimidine (7.00 g, 41.0 mmol), and the mixture was stirred for 1 hour at −78° C. Ethyl formate (5.03 mL, 61.5 mmol) was added at −78° C. to the reaction solution, the mixture was stirred for 3 hours, water was then poured into the reaction solution, and the mixture was extracted with dichloromethane. The extract was dried over anhydrous magnesium sulfate, and the solvent was distilled off at reduced pressure. The residue was purified by silica gel column chromatography (ethyl acetate:hexane=1:4) to give 3.02 g of the titled compound (yield 37%) in solid form.

¹H-NMR (CDCl₃) δ: 8.23 (1H, s), 9.11 (1H, s), 10.27 (1H, s).

### Reference Example 395

### (4-Chlorothieno[3,2-d]pyrimidin-6-yl)methanol

Sodium borohydride (690 mg, 18.3 mmol) was added at 0° C. to an ethanol (50.7 mL) solution of 4-chlorothieno[3,2-d]pyrimidine-6-carbaldehyde (3.02 g, 15.2 mmol) obtained in Reference Example 394, and the mixture was stirred for 2 hours at 0° C. Water was poured into the reaction solution, and the mixture was extracted with ethyl acetate. The extract was dried over anhydrous magnesium sulfate, and the solvent was distilled off at reduced pressure to give 3.00 g of the titled compound (yield 98%) in solid form. The resulting solid was used without further modification in the following reaction.

¹H-NMR (CDCl₃) δ: 3.29 (1H, br s), 5.09 (2H, s), 7.43 (1H, s), 8.93 (1H, s).

### Reference Example 396

### Ethyl 3-[6-(hydroxymethyl)thieno[3,2-d]pyrimidin-4-yl]benzoate

A THF (50 mL)-water (0.80 mL) mixture of (4-chlorothieno[3,2-d]-pyrimidin-6-yl)methanol (3.0 g, 15.0 mmol) obtained in Reference Example 395, [3-(ethoxycarbonyl)phenyl]boronic acid (4.35 g, 22.4 mmol), potassium phosphate (9.52 g, 44.9 mmol), palladium acetate (336 mg, 1.50 mmol), and 2-(dicyclohexylphosphino)-2′,4′,6′-triisopropyl-1,1′-biphenyl (713 mg, 1.50 mmol) was stirred for 17 hours at 60° C. in a nitrogen atmosphere. The reaction solution was cooled to room temperature, then diluted with dichloromethane, washed with water, and dried over anhydrous sodium sulfate, and the solvent was distilled off at reduced pressure. The residue was purified by silica gel column chromatography (ethyl acetate) to give 1.20 g of the titled compound (yield 26%) in solid form.

¹H-NMR (CDCl₃) δ: 1.44 (3H, t, J=7.2 Hz), 2.17 (1H, br s), 4.45 (2H, q, J=7.2 Hz), 5.11 (2H, d, J=6.0 Hz), 7.51 (1H, s), 7.67 (1H, t, J=7.8 Hz), 8.25 (1H, d, J=7.6 Hz), 8.39 (1H, d, J=8.8 Hz), 8.87 (1H, s), 9.29 (1H, s).

### Reference Example 397

### Ethyl 3-[6-[3-(trifluoromethyl)benzyl]thieno[3,2-d]pyrimidin-4-yl]benzoate

Thionyl chloride (0.334 mL, 4.58 mmol) was added dropwise at 0° C. to a dichloromethane solution (38 mL) of ethyl 3-[6-(hydroxymethyl)thieno[3,2-d]pyrimidin-4-yl]benzoate (1.20 g, 3.82 mmol) obtained in Reference Example 396, the mixture was stirred for 5 hours at room temperature, and the reaction solution was then concentrated to give ethyl 3-[6-(chloromethyl)thieno[3,2-d]pyrimidin-4-yl]benzoate. A THF (5.0 mL)-water (0.08 mL) mixture of the resulting ethyl 3-[6-(chloromethyl)thieno[3,2-d]pyrimidin-4-yl]benzoate (500 mg, 1.50 mmol), 3-trifluoromethylphenylboronic acid (428 mg, 2.25 mmol), cesium carbonate (1.47 g, 4.51 mmol), and 1,1-bis-(diphenylphosphino)-ferrocene palladium dichloride (61.0 mg, 0.080 mmol) was stirred for 17 hours at 60° C. in a nitrogen atmosphere. The reaction solution was cooled to room temperature, water was then added, and the mixture was extracted with dichloromethane. The extract was dried over anhydrous magnesium sulfate, and the solvent was distilled off at reduced pressure. The residue was purified by silica gel column chromatography (ethyl acetate:hexane=1:2) to give 371 mg of the titled compound (yield 56%) in solid form.

¹H-NMR (CDCl₃) δ: 1.42 (3H, t, J=7.4 Hz), 4.41 (2H, s), 4.43 (2H, q, J=7.2 Hz), 7.36 (1H, s), 7.52 (2H, s), 7.58 (2H, s), 7.65 (1H, t, J=7.8 Hz), 8.23 (1H, d, J=7.6 Hz), 8.33 (1H, d, J=8.0 Hz), 8.82 (1H, s), 9.26 (1H, s).

### Reference Example 398

### 3-[6-[3-(Trifluoromethyl)benzyl]thieno[3,2-d]pyrimidin-4-yl]benzoic acid

An ethanol (3.58 mL) solution of ethyl 3-[6-[3-(trifluoromethyl)benzyl]thieno[3,2-d]pyrimidin-4-yl]benzoate (317 mg, 0.716 mmol) obtained in Reference Example 397 and 2 N sodium hydroxide aqueous solution (1.08 mL, 2.15 mmol), was stirred for 17 hours at room temperature. The reaction solution was neutralized with 10% citric acid aqueous solution, and the resulting solids were filtered off and washed with water. The resulting solids were purified by HPLC (LC: Agilent 1100; column: 50×300 mm C18; eluate A: 0.01% trifluoroacetic acid acetonitrile solution; eluate B: 0.01% trifluoroacetic acid aqueous solution; eluted with 30% eluate A to 95% eluate A) to give 43.0 mg of the titled compound (yield 15%) in solid form.

¹H-NMR (MeOH-d₄) δ: 4.53 (2H, s), 7.41 (1H, s), 7.53-7.65 (3H, m), 7.68-7.72 (2H, m), 8.21-8.24 (1H, m), 8.34-8.36 (1H, m), 8.78 (1H, dd, J=1.6, 1.6 Hz), 9.16 (1H, s).

### Reference Example 399

### 3-[2-(3-Chloro-5-fluorobenzyl)-1-benzothiophen-7-yl]benzoic acid

Ethyl 3-[2-(3-chloro-5-fluorobenzyl)-1-benzothiophen-7-yl]benzoate obtained in Reference Example 206 was used in the same manner as in Reference Example 5 to obtain the titled compound. Yield: 70%.

¹H-NMR (DMSO-d₆) δ: 4.30 (2H, s), 7.17-7.25 (1H, m), 7.27-7.34 (2H, m), 7.36-7.43 (2H, m), 7.45-7.53 (1H, m), 7.62-7.71 (1H, m), 7.83 (1H, dd, J=7.7, 0.9 Hz), 7.87-7.94 (1H, m), 7.97-8.04 (1H, m), 8.22 (1H, d, J=8.1 Hz), 13.2(1H, br s).

### Reference Example 400

### Ethyl 3-[2-(bromomethyl)-4-fluoro-1-benzothiophen-7-yl]benzoate

Ethyl 3-[4-fluoro-2-(hydroxymethyl)-1-benzothiophene-7-yl]benzoate obtained in Reference Example 170, and phosphorus tribromide was used in the same manner as in Reference Example 187 to obtain the titled compound. Yield: 60%, white solid.

¹H-NMR (CDCl₃) δ: 1.42 (3H, t, J=7.2 Hz), 4.32 (2H, q, J=7.2 Hz), 4.76 (2H, s), 7.13 (1H, dd, J=8.1, 9.6 Hz), 7.34 (1H, dd, J=4.8, 8.1 Hz), 7.50-7.62 (2H, m), 7.82-7.89 (1H, m), 8.07-8.14 (1H, m), 8.29-8.34 (1H, m).

### Reference Example 401

### Ethyl 3-[2-(3-chloro-5-fluorobenzyl)-4-fluoro-1-benzothiophen-7-yl]benzoate

A mixture of ethyl 3-[2-(bromomethyl)-4-fluoro-1-benzothiophen-7-yl]benzoate (1.15 g, 2.92 mmol) obtained in Reference Example 400, 3-chloro-5-fluorophenylboric acid (0.76 g, 4.38 mmol), tetrakis(triphenylphosphine)palladium (169 mg, 0.146 mmol), 2 N sodium carbonate aqueous solution (6.0 mL), ethanol (4.0 mL), and 1,2-dimethoxyethane (15 mL) was stirred for 15 hours at 80° C. in an argon atmosphere. After cooling to room temperature, the reaction solution was partitioned with ethyl acetate and water. The organic layer was washed with saturated brine and dried over anhydrous magnesium sulfate, and the solvent was then distilled off at reduced pressure. The residue was purified by silica gel column chromatography (hexane-ethyl acetate=100:0-92/8) to give 1.03 g of the titled compound (yield 80%) in the form of white solids.

¹H-NMR (CDCl₃) δ: 1.40 (3H, t, J=7.2 Hz), 4.18 (2H, s), 4.40 (2H, q, J=7.2 Hz), 6.84-6.93 (1H, m), 6.94-7.02 (1H, m), 7.03-7.18 (2H, m), 7.24-7.87 (1H, m), 8.03-8.12 (1H, m), 8.26-8.32 (1H, m).

### Reference Example 402

### 3-[2-(3-Chloro-5-fluorobenzyl)-4-fluoro-1-benzothiophen-7-yl]benzoic acid

A mixture of ethyl 3-[2-(3-chloro-5-fluorobenzyl)-4-fluoro-1-benzothiophen-7-yl]benzoate (1.03 g, 2.33 mmol) obtained in Reference Example 401, 4 N sodium hydroxide aqueous solution (1.5 mL), ethanol (6.0 mL), and THF (6.0 mL) was stirred for 1 hour at 60° C. After cooling to room temperature, the reaction solution was diluted with I N hydrochloric acid and extracted with ethyl acetate. The extract was washed with saturated brine and dried over anhydrous magnesium sulfate, and the solvent was then distilled off at reduced pressure to give 0.84 g of the titled compound (yield 87%) in the form of light yellow solids.

¹H-NMR (DMSO-d₆) δ: 4.34 (2H, s), 7.20-7.38 (4H, m), 7.40-7.52 (2H, m), 7.97-8.05 (1H, m), 8.16-8.22 (1H, m), 13.11 (1H, br s).

### Reference Example 403

Ethyl 3-fluoro-5-[4-fluoro-2-(hydroxymethyl)-1-benzothiophen-7-yl]benzoate (7-Bromo-4-fluoro-1-benzothiophene-2-yl)methanol obtained in Reference Example 164, and ethyl 3-fluoro-5-(4,4,5,5-tetramethyl-1,3,2-dioxaborolane-2-yl)benzoate obtained in Reference Example 225 were used in the same manner as in Reference Example 401 to obtain the titled compound. Yield: 55%, pale yellow solid.

¹H-NMR (CDCl₃) δ: 1.41 (3H, t, J=7.2 Hz), 1.97 (1H, t, J=6.0 Hz), 4.42 (2H, q, J=7.2 Hz), 4.95 (2H, d, J=6.0 Hz), 7.13 (1H, dd, J=8.1, 9.6 Hz), 7.31 (1H, dd, J=4.8, 8.1 Hz), 7.40-7.45 (1H, m), 7.54-7.62 (1H, m), 7.73-7.80 (1H, m), 8.11-8.16 (1H, m).

### Reference Example 404

### Ethyl 3-[2-(bromomethyl )-4-fluoro-1-benzothiophen-7-yl]-5-fluorobenzoate

Ethyl 3-fluoro-5-[4-fluoro-2-(hydroxymethyl)-1-benzothiophen-7-yl]benzoate obtained in Reference Example 403, and phosphorus tribromide were used in the same manner as in Reference Example 187 to obtain the titled compound. Yield: 48%, white solid.

¹H-NMR (CDCl₃) δ: 1.42 (3H, t, J=7.2 Hz), 4.42 (2H, q, J=7.2 Hz), 4.76 (2H, s), 7.13 (1H, dd, J=8.1, 9.3 Hz), 7.34 (1H, dd, J=4.8, 8.1 Hz), 7.51-7.60 (2H, m), 7.77 (1H, ddd, J=1.5, 2.4, 9.0 Hz), 8.10-8.14 (1H, m).

### Reference Example 405

Ethyl 3-[2-(3-chloro-5-fluorobenzyl)-4-fluoro-1-benzothiophen-7-yl]-5-fluorobenzoate Ethyl 3-[2-(bromomethyl)-4-fluoro-1-benzothiophen-7-yl]-5-fluorobenzoate obtained in Reference Example 404, and 3-chloro-5-fluorophenylboric acid were used in the same manner as in Reference Example 401 to obtain the titled compound. Yield: 35%, colorless oily substance.

¹H-NMR (CDCl₃) δ: 1.40 (3H, t, J=7.2 Hz), 4.19 (2H, s), 4.40 (2H, q, J=7.2 Hz), 6.85-6.92 (1H, m), 6.95-7.02 (1H, m), 7.05-7.16 (2H, m), 7.22-7.32 (2H, m), 7.53 (1H, ddd, J=1.5, 2.4, 9.3 Hz), 7.74 (1H, ddd, J=1.5, 2.4, 9.0 Hz), 8.09-8.12 (1H, m).

### Reference Example 406

### 3-[2-(3-Chloro-5-fluorobenzyl)-4-fluoro-1-benzothiophen-7-yl]-5-fluorobenzoic acid

Ethyl 3-[2-(3-chloro-5-fluorobenzyl)-4-fluoro-1-benzothiophen-7-yl]-5-fluorobenzoate obtained in Reference Example 405 was used in the same manner as in Reference Example 402 to obtain the titled compound. Yield: 60%, colorless oily substance.

¹H-NMR (CDCl₃) δ: 4.19 (2H, s), 6.84-6.92 (1H, m), 6.94-7.02 (1H, m), 7.04-7.34 (4H, m), 7.55-7.63 (1H, m), 7.76-7.84 (1H, m), 8.15-8.20 (1H, m).

### Reference Example 407

### Ethyl 2-fluoro-5-(4,4,5,5-tetramethyl-1,3,2-dioxaborolan-2-yl]benzoate

A DMF (35 mL) solution of ethyl 5-bromo-2-fluorobenzoate (2.89 g, 11.7 mmol), bis(pinacolato)diboron (3.56 g, 14.0 mmol), (1,1-bis(diphenylphosphino)ferrocene)dichloropalladium (II) complex with dichloromethane (478 mg, 0.585 mmol), and potassium acetate (3.50 g, 35.7 mmol) was stirred for 15 hours at 80° C. in an argon atmosphere. After cooling to room temperature, the reaction solution was partitioned with ethyl acetate and water. The organic layer was washed with saturated brine and dried over anhydrous magnesium sulfate, and the solvent was then distilled off at reduced pressure. The residue was purified by silica gel column chromatography (hexane-ethyl acetate=98:2-80/20) to give 3.54 mg of the titled compound (yield 99%) in the form of a light tan oily substance.

¹H-NMR (CDCl₃) δ: 1.35 (12 h, s), 1.40 (3H, t, J=7.2 Hz), 4.39 (2H, q, J=7.2 Hz), 7.12 (1H, dd, J=8.4, 10.8 Hz), 7.86-7.98 (1H, m), 8.34 (1H, dd, J=1.8, 7.8 Hz).

### Reference Example 408

### Ethyl 2-fluoro-5-[4-fluoro-2-(hydroxymethyl)-1-benzothiophen-7-yl]benzoate

(7-Bromo-4-fluoro-1-benzothiophene-2-yl)methanol obtained in Reference Example 164, and ethyl 2-fluoro-5-(4,4,5,5-tetramethyl-1,3,2-dioxaborolan-2-yl)benzoate obtained in Reference Example 407 were used in the same manner as in Reference Example 401 to obtain the titled compound. Yield: 65%, white solid.

¹H-NMR (CDCl₃) δ: 1.41 (3H, t, J=7.2 Hz), 2.02 (1H, t, J=6.0 Hz), 4.42 (2H, q, J=7.2 Hz), 4.94(1H, d, J=6.0 Hz), 7.11 (1H, dd, J=8.4, 9.9 Hz), 7.20-7.32 (2H, m), 7.39-7.43 (1H, m), 7.80 (1H, ddd, J=2.4, 4.5, 8.4 Hz), 8.19 (1H, dd, J=2.4, 6.9 Hz).

### Reference Example 409

### Ethyl 5-[2-(bromomethyl)-4-fluoro-1-benzothiophen-7-yl]-2-fluorobenzoate

Ethyl 2-fluoro-5-[4-fluoro-2-(hydroxymethyl)-1-benzothiophen-7-yl]benzoate obtained in Reference Example 408, and phosphorus tribromide were used in the same manner as in Reference Example 187 to obtain the titled compound. Yield: 68%, colorless solid.

¹H-NMR (CDCl₃) δ: 1.42 (3H, t, J=7.2 Hz), 4.43 (2H, q, J=7.2 Hz), 4.76 (2H, s), 7.12 (1H, dd, J=7.8, 9.3 Hz), 7.22-7.34 (2H, m), 7.51 (1H, s), 7.79 (1H, ddd, J=2.4, 4.8, 8.4 Hz), 8.19 (1H, dd, J=2.4, 6.6 Hz).

### Reference Example 410

### Ethyl 5-[2-(3-chloro-5-fluorobenzyl)-4-fluoro-1-benzothiophen-7-yl]-2-fluorobenzoate

Ethyl 5-[2-(bromomethyl)-4-fluoro-1-benzothiophen-7-yl]-2-fluorobenzoate obtained in Reference Example 409, and 3-chloro-5-fluorophenylboric acid were used in the same manner as in Reference Example 401 to obtain the titled compound. Yield: 73%, colorless oily substance.

¹H-NMR (CDCl₃) δ: 1.40 (3H, t, J=7.2 Hz), 4.19 (2H, s), 4.41 (2H, q, J=7.2 Hz), 6.85-6.92 (1H, m), 6.95-7.02 (1H, m), 7.04-7.15 (2H, m), 7.19-7.30 (3H, m), 7.76 (1H, ddd, J=2.4, 4.5, 8.7 Hz), 8.17 (1H, dd, J=2.4, 7.2 Hz).

### Reference Example 411

### 5-[2-(3-Chloro-5-fluorobenzyl)-4-fluoro-1-benzothiophen-7-yl]-2-fluorobenzoic acid

Ethyl 5-[2-(3-chloro-5-fluorobenzyl)-4-fluoro-1-benzothiophen-7-yl]-2-fluorobenzoate obtained in Reference Example 410 was used in the same manner as in Reference Example 402 to obtain the titled compound. Yield: 76%, pale yellow oily substance.

¹H-NMR (CDCl₃) δ: 4.34 (2H, s), 7.20-7.37 (4H, m), 7.38-7.53 (3H, m), 7.89 (1H, ddd, J=2.4, 4.5, 8.7 Hz), 8.09 (1H, dd, J=2.4, 6.9 Hz), 13.47 (1H, br s).

### Reference Example 412

### N-(2-Amino-2-oxoethyl)-3-[2-(hydroxymethyl)-1-benzothiophen-7-yl]benzamide

Ethyl 3-[2-(hydroxymethyl)-1-benzothiophene-7-yl]benzoate obtained in Reference Example 168 was used in the same manner as in Reference Examples 177, and 178 to give 3-[2-(hydroxymethyl)-1-benzothiophene-7-yl]benzoic acid crude product, and the resulting crude product of 3-[2-(hydroxymethyl)-1-benzothiophene-7-yl]benzoic acid, and glycinamide hydrochloride were used to give the titled compound. Yield: 77%. White solid.

¹H-NMR (CDCl₃) δ: 2.10-2.19 (1H, m), 3.50 (1H, d, J=5.5 Hz), 4.20 (1H, d, J=5.2 Hz), 4.93 (2H, d, J=6.0 Hz), 5.50 (1H, br s), 6.07 (1H, br s), 7.40 (1H, br s), 7.30 (1H, s), 7.33-7.38 (1H, m), 7.41-7.48 (1H, m), 7.53-7.61 (1H, m), 7.74 (1H, dd, J=8.0, 1.1 Hz), 7.81-7.91 (2H, m), 8.14 (1H, t, J=1.8 Hz).

### Reference Example 413

### Ethyl 3-[2-(4-fluoro-3-methoxybenzyl)-1-benzothiophen-7-yl]benzoate

Ethyl 3-[2-(bromomethyl)-1-benzothiophene-7-yl]benzoate obtained in Reference Example 179, and (4-fluoro-3-methoxyphenyl)boronic acid were used in the same manner as in Reference Example 200 to obtain the titled compound. Yield: 72%. Oily substance.

¹H-NMR (CDCl₃) δ: 1.40 (3H, t, J=7.0 Hz), 3.85 (3H, s), 4.15-4.23 (2H, m), 4.40 (2H, q, J=7.1 Hz), 6.77-6.84 (1H, m), 6.86 (1H, dd, J=8.0, 2.2 Hz), 7.00 (1H, dd, J=11.3, 8.2 Hz), 7.09 (1H, s), 7.29-7.34 (1H, m), 7.43 (1H, t, J=7.5 Hz), 7.54 (1H, t, J=7.7 Hz), 7.68 (1H, dd, J=8.0, 1.1 Hz), 7.88 (1H, dt, J=7.7, 1.5 Hz), 8.07 (1H, dt, J=7.8, 1.5 Hz), 8.34 (1H, t, J=1.8 Hz).

### Reference Example 414

### Ethyl 3-[2-(2,3-dihydro-1-benzofuran-5-ylmethyl )-1-benzothiophen-7-yl]benzoate

Ethyl 3-[2-(bromomethyl)-1-benzothiophene-7-yl]benzoate obtained in Reference Example 179, and 2,3-dihydro-1-benzofuran-5-ylboronic acid were used in the same manner as in Reference Example 200 to obtain the titled compound. Yield: 57%. Oily substance.

¹H-NMR (CDCl₃) δ: 1.40 (3H, t, J=7.1 Hz), 3.17 (2H, t, J=8.7 Hz), 4.14 (2H, s), 4.40 (2H, q, J=7.1 Hz), 4.54 (2H, t, J=8.8 Hz), 6.71 (1H, d, J=8.2 Hz), 7.02 (1H, dd, J=8.0, 1.6 Hz), 7.09 (2H, s), 7.28-7.33 (1H, m), 7.41 (1H, t, J=7.6 Hz), 7.53 (1H, t, J=7.7 Hz), 7.67 (1H, dd, J=7.7, 1.1 Hz), 7.88.(1H, dt, J=7.8, 1.4 Hz), 8.06 (1H, dt, J=7.9, 1.4 Hz), 8.33 (1H, t, J=1.8 Hz).

### Reference Example 415

### Ethyl 3-[2-(3-cyanobenzyl)-1-benzothiophen-7-yl]benzoate

Ethyl 3-[2-(bromomethyl)-1-benzothiophene-7-yl]benzoate obtained in Reference Example 179, and (3-cyanophenyl)boronic acid were used in the same manner as in Reference Example 200 to obtain the titled compound. Yield: 68%. Oily substance.

¹H-NMR (CDCl₃) δ: 1.40 (3H, t, J=7.1 Hz), 4.25 (2H, s), 4.40 (2H, q, J=7.1 Hz), 7.13 (1H, s), 7.32-7.36 (1H, m), 7.38-7.58 (6H, m), 7.70 (1H, dd, J=7.7, 1.1 Hz), 7.87 (1H, dt, J=7.7, 1.5 Hz), 8.08 (1H, dt, J=7.8, 1.5 Hz), 8.34 (1H, t, J=1.8 Hz).

### Reference Example 416

### Ethyl 3-[2-[(5-chlorothiophen-2-yl)methyl]-1-benzothiophen-7-yl]benzoate

Ethyl 3-[2-(bromomethyl)-1-benzothiophene-7-yl]benzoate obtained in Reference Example 179, and (5-chlorothiophene-2-yl)boronic acid were used in the same manner as in Reference Example 200 to obtain the titled compound. Yield: 63%. Oily substance.

¹H-NMR (CDCl₃) δ: 1.39 (3H, t, J=7.0 Hz), 4.30 (2H, s), 4.39 (2H, q, J=7.1 Hz), 6.66-6.69 (1H, m), 6.71-6.74 (1H, m), 7.16 (1H, t, J=1.0 Hz), 7.30-7.34 (1H, m), 7.42 (1H, t, J=7.7 Hz), 7.53 (1H, t, J=7.7 Hz), 7.68 (1H, dd, J=7.8, 1.2 Hz), 7.84-7.89 (1H, m), 8.06 (1H, dt, J=7.8, 1.5 Hz), 8.32-8.34 (1H, m).

### Reference Example 417

### Ethyl 3-[2-[3-(dimethylamino)benzyl]-1-benzothiophen-7-yl]benzoate

Ethyl 3-[2-(bromomethyl)-1-benzothiophene-7-yl]benzoate obtained in Reference Example 179, and [3-(dimethylamino)phenyl]boronic acid were used in the same manner as in Reference Example 200 to obtain the titled compound. Yield: 87%. Oily substance.

¹H-NMR (CDCl₃) δ: 1.40 (3H, t, J=7.1 Hz), 2.93 (6H, s), 4.17 (2H, s), 4.40 (2H, q, J=7.1 Hz), 6.59-6.68 (3H, m), 7.10 (1H, t, J=1.1 Hz), 7.14-7.22 (1H, m), 7.30 (1H, dd, J=7.4, 1.1 Hz), 7.37-7.44 (1H, m), 7.50-7.57 (1H, m), 7.66 (1H, dd, J=7.7, 1.1 Hz), 7.89 (1H, ddd, J=7.7, 1.9, 1.1 Hz), 8.03-8.09 (1H, m), 8.32-8.36 (1H, m).

### Reference Example 418

### Ethyl 3-[2-[3-(hydroxymethyl)benzyl]-1-benzothiophen-7-yl]benzoate

Ethyl 3-[2-(bromomethyl)-1-benzothiophene-7-yl]benzoate obtained in Reference Example 179, and [3-(hydroxymethyl)phenyl]boronic acid were used in the same manner as in Reference Example 200 to obtain the titled compound. Yield: 78%. Oily substance.

¹H-NMR (CDCl₃) δ: 1.40 (3H, t, J=7.1 Hz), 1.65 (1H, t, J=5.9 Hz), 4.23 (2H, s), 4.40 (2H, q, J=7.1 Hz), 4.68 (2H, d, J=5.8 Hz), 7.11 (1H, t, J=1.0 Hz), 7.18-7.26 (2H, m), 7.27-7.35 (3H, m), 7.39-7.45 (1H, m), 7.50-7.57 (1H, m), 7.68 (1H, dd, J=7.7, 1.1 Hz), 7.88 (1H, dq, J=7.7, 1.0 Hz), 8.06 (1H, dt, J=7.7, 1.4 Hz), 8.34 (1H, t, J=1.8 Hz).

### Reference Example 419

### Ethyl 3-[2-(3-chloro-5-fluorobenzyl)-3-methyl-1-benzothiophen-7-yl]benzoate

Ethyl 3-[2-(bromomethyl)-3-methyl-1-benzothiophene-7-yl]benzoate obtained in Reference Example 180, and (3-chloro-5-fluorophenyl)boronic acid were used in the same manner as in Working Example 200 to obtain the titled compound. Yield: 71%. Oily substance.

¹H-NMR (CDCl₃) δ: 1.38 (3H, t, J=7.1 Hz), 2.39 (3H, s), 4.15 (2H, s), 4.38 (2H, q, J=7.1 Hz), 6.76-6.82 (1H, m), 6.91 (1H, dt, J=8.4, 2.0 Hz), 6.98 (1H, s), 7.35 (1H, dd, J=7.4, 1.1 Hz), 7.45-7.57 (2H, m), 7.66 (1H, dd, J=7.8, 1.0 Hz), 7.86 (1H, dt, J=7.8, 1.6 Hz), 8.05 (1H, dt, J=7.7, 1.4 Hz), 8.32 (1H, t, J=1.8 Hz).

### Reference Example 420

### Ethyl 3-[2-([[4-(trifluoromethyl)pyridin-2-yl]oxy]methyl)-1-benzothiophen-7-yl]benzoate

4-(Trifluoromethyl)pyridine-2-ol (142 mg, 0.871 mmol), triphenylphosphine (274 mg, 1.045 mmol), and diethyl azocarboxylate (475 μL, 1.045 mmol) were added while cooled on ice to a THF solution (8 mL) of ethyl 3-[2-(hydroxymethyl)-1-benzothiophen-7-yl]benzoate (271 mg, 0.871 mmol) obtained in Reference Example 168, and the mixture was stirred for 24 hours at room temperature. The reaction solution was concentrated at reduced pressure, and the residue was purified by column chromatography (NH, hexane/ethyl acetate: 95/5→67/33; SiO₂, hexane/ethyl acetate: 75/25→50/50) to give the titled compound. Yield: 47%. Oily substance.

¹H-NMR (CDCl₃) δ: 1.41 (3H, t, J=7.1 Hz), 4.41 (2H, q, J=6.9 Hz), 5.37 (2H, s), 6.29 (1H, dd, J=7.1, 1.9 Hz), 6.87 (1H, s), 7.36-7.41 (1H, m), 7.42-7.52 (3H, m), 7.56 (1H, t, J=7.7 Hz), 7.76 (1H, dd, J=7.8, 1.2 Hz), 7.84-7.89 (1H, m), 8.09 (1H, dt, J=7.8, 1.2 Hz), 8.31 (1H, t, J=1.8 Hz).

### Reference Example 421

### Ethyl 3-[2-[(diethoxyphosphoryl)methyl]-1-benzothiophen-7-yl]benzoate

A mixture of ethyl 3-[2-(bromomethyl)-1-benzothiophen-7-yl]benzoate (1.0 g, 2.665 mmol) obtained in Reference Example 179 and triethyl phosphite (503 μL, 2.931 mmol) was heated to reflux for 4 hours. The reaction solution was concentrated at reduced pressure, and the residue was purified by silica gel column chromatography (hexane/ethyl acetate: 50/50→25/75) to give the titled compound. Yield: 88%. Oily substance.

¹H-NMR (CDCl₃) δ: 1.30 (6H, t, J 7.0 Hz), 1.41 (3H, t, J=7.1 Hz), 3.36-3.51 (2H, m), 4.04-4.19 (4H, m), 4.41 (2H, q, J 7.1 Hz), 7.29-7.39 (2H, m), 7.40-7.49 (1H, m), 7.56 (1H, t, J=7.8 Hz), 7.71 (1H, d ,J=7.7 Hz), 7.91 (1H, dt, J=7.7, 1.5 Hz), 8.09 (1H, dt, J=7.9, 1.4 Hz), 8.36 (1H, t, J=1.8 Hz).

### Reference Example 422

### Ethyl 3-(2-[(E)-2-[3-(trifluoromethyl)phenyl]ethenyl]-1-benzothiophen-7-yl]benzoate

Sodium hydride (112 mg, 2.802 mmol) was added while cooled on ice to a THF solution (17 mL) of ethyl 3-[2-[(diethoxyphosphoryl)methyl]-1-benzothiophen-7-yl]benzoate (1.01 g, 2.335 mmol) obtained in Reference Example 421, and the mixture was stirred for 15 min. A THF solution (6 mL) of 3-(trifluoromethyl)benzaldehyde (403 μL, 3.036 mmol) was added to the resulting reaction solution, and the mixture was stirred for 10 min while cooled on ice and then stirred for 1 hour at room temperature. Sodium hydride (112 mg, 2.802 mmol) was added, and the reaction solution was then stirred for 1 hour at room temperature and then stirred for 90 min at 40° C. The addition of saturated ammonium chloride aqueous solution to the reaction solution was followed by extraction with ethyl acetate. The extract was washed with saturated brine and dried over sodium sulfate. The reaction solution was concentrated at reduced pressure, and the residue was then purified by silica gel column chromatography (hexane/ethyl acetate: 95/5→80/20) to give the titled compound. Yield: 89%. White solids.

¹H-NMR (CDCl₃) δ: 1.43 (3H, t, J=7.1 Hz), 4.44 (2H, q, J=7.0 Hz), 7.00 (1H, d, J=15.9 Hz), 7.33-7.56 (6H, m), 7.57-7.68 (2H, m), 7.71-7.78 (2H, m), 7.94 (1H, ddd, J=7.7, 1.9, 1.1 Hz), 8.13 (1H, dt, J=7.7, 1.5 Hz), 8.37-8.41 (1H, m).

### Reference Example 423

### Ethyl 3-(2-[2-[3-(trifluoromethyl)phenyl]ethyl]-1-benzothiophen-7-yl]benzoate

Palladium-carbon (94 mg) was added to a THF solution (20 mL) of ethyl 3-(2-[(E)-2-[3-(trifluoromethyl)phenyl]ethenyl]-1-benzothiophen-7-yl]benzoate (940 mg, 2.078 mmol) obtained in Reference Example 422, and the mixture was stirred for 3 hours at room temperature in a hydrogen atmosphere. The reaction solution was filtered using celite, and the filtrate was concentrated at reduced pressure. The residue was purified by silica gel column chromatography (hexane/ethyl acetate: 95/5→80/20) to give the titled compound. Yield: 98%. Oily substance.

¹H-NMR (CDCl₃) δ: 1.42 (3H, t, J=7.1 Hz), 3.06-3.16 (2H, m), 3.18-3.28 (2H, m), 4.42 (2H, q, J=7.1 Hz), 7.06 (1H, s), 7.30-7.35 (1H, m), 7.36-7.50 (5H, m), 7.53-7.61 (1H, m), 7.67 (1H, dd, J=8.0, 1.1 Hz), 7.88-7.94 (1H, m), 8.07-8.13 (1H, m), 8.36-8.40 (1H, m).

### Reference Example 424

### Ethyl 3-[2-([[3-(trifluoromethyl)phenyl]amino]methyl)-1-benzothiophen-7-yl]benzoate

3-(Trifluoromethyl)aniline (938 μL, 7.568 mmol) and potassium carbonate (1.05 g, 7.568 mmol) were added to a DMF (30 mL) solution of ethyl 3-[2-(bromomethyl)-1-benzothiophen-7-yl]benzoate (1.42 g, 3.784 mmol) obtained in Reference Example 179, and the mixture was heated to reflux over night at 80° C. The reaction solution was cooled to room temperature, neutralized with ethyl acetate and water, and extracted with ethyl acetate. The resulting organic layer was washed with water and saturated brine, and was then dried over sodium sulfate. The solvent was distilled off at reduced pressure, and the residue was purified by silica gel column chromatography (hexane/ethyl acetate: 95/5→80/20) to give the titled compound. Yield: 88%. Oily substance.

¹H-NMR (CDCl₃) δ: 1.40 (3H, t, J=7.1 Hz), 4.41 (3H, q, J=7.0 Hz), 4.63 (2H, dd, J=5.8, 1.1 Hz), 6.80 (1H, dd, J=8.2, 2.5 Hz), 6.89 (1H, s), 6.97 (1H, dt, J=7.7, 0.8 Hz), 7.21-7.29 (1H, m), 7.33 (1H, t, J=1.1 Hz), 7.36 (1H, dd, J=7.4, 1.1 Hz), 7.42-7.49 (1H, m), 7.52-7.59 (1H, m), 7.72 (1H, dd, J=7.8, 1.2 Hz), 7.89 (1H, ddd, J=7.7, 1.9, 1.1 Hz), 8.09 (1H, dt, J=7.8, 1.3 Hz), 8.36 (1H, t, J=1.6 Hz).

### Reference Example 425

### Ethyl 3-[2-([methyl[3-(trifluoromethyl)phenyl]amino]methyl)-1-benzothiophen-7-yl]benzoate

Sodium hydride (339 mg, 8.474 mmol) was added to a DMF (40 mL) solution of ethyl 3-[2-([[3-(trifluoromethyl)phenyl]amino]methyl)-1-benzothiophen-7-yl]benzoate (1.93 g, 4.237 mmol) obtained in Reference Example 424. The mixture was stirred for 30 min at room temperature, iodomethane (2.64 mL, 42.37 mmol) was then added, and the mixture was stirred for another 90 min at room temperature. The addition of water to the reaction solution was followed by extraction with ethyl acetate. The resulting organic layer was washed with saturated brine, and was then dried over sodium sulfate. The solvent was distilled off at reduced pressure, and the residue was purified by silica gel column chromatography (hexane/ethyl acetate) to give the titled compound. Yield: 3%. Oily substance.

¹H-NMR (CDCl₃) δ: 1.40 (3H, t, J=7.1 Hz), 3.11 (3H, s), 4.40 (2H, q, J=7.0 Hz), 4.77 (2H, s), 6.89-7.04 (3H, m), 7.21 (1H, s), 7.26-7.36 (2H, m), 7.44 (1H, t, J=7.5 Hz), 7.52-7.59 (1H, m), 7.69 (1H, dd, J=7.7, 1.1 Hz), 7.88 (1H, dt, J=7.7, 1.6 Hz), 8.04-8.12 (1H, m), 8.34 (1H, t, J=1.8 Hz).

### Reference Example 426

### 2-Fluoro-3-methoxybenzaldehyde

sec-Butyllithium (100 mL, 106 mmol) was added dropwise over a period of 25 min at −78° C. to a THF solution (450 mL) of 1-fluoro-2-methoxybenzene (11.2 mL, 100 mmol) and N,N,N′N′-tetramethylethylenediamine (18.1 mL, 120 mmol), and the mixture was stirred for 2 hours. DMF (9.2 mL, 120 mmol) was added, and the mixture was stirred for 30 min. Acetic acid (5. mL) and water (200 mL) were added, the reaction solution was returned to room temperature, and it was extracted with ethyl acetate. The resulting organic layer was washed with 1N hydrochloric acid and saturated brine, and was then dried over sodium sulfate. The solvent was distilled off at reduced pressure, and the residue was purified by silica gel column chromatography (hexane/ethyl acetate: 95/5→80/20) to give the titled compound. Yield: 65%. White solids.

¹H-NMR (CDCl₃) δ: 3.94 (3H, s), 7.14-7.25 (2H, m), 7.37-7.46 (1H, m), 10.39 (1H, s).

### Reference Example 427

### 7-Methoxy-1-benzothiophene-2-carboxylic acid

Ethyl sulfanyl acetate (3.89 mL, 35.50 mmol) and potassium carbonate (8.17 g, 59.17 mmol) were added to a DMF (200 mL) solution of 2-fluoro-3-methoxybenzaldehyde (4.56 g, 29.58 mmol) obtained in Reference Example 426, and the mixture was stirred over night at 80° C. The addition of water to the reaction solution was followed by extraction with ethyl acetate. The resulting organic layer was washed with water and saturated brine, and was then dried over sodium sulfate. The solvent was distilled off at reduced pressure to give ethyl 7-methoxy-1-benzothiophene-2-carboxylate crude product (7.27 g). A 2N sodium hydroxide aqueous solution (44.4 mL, 88.74 mmol) was added to a THF/methanol (100 mL/50 mL) solution of the resulting ethyl 7-methoxy-1-benzothiophene-2-carboxylate crude product (7.27 g), and the mixture was stirred over night at room temperature. The reaction solution was concentrated at reduced pressure, and the addition of 1 N hydrochloric acid (120 mL) was followed by extraction with ethyl acetate. The extract was washed with saturated brine and dried over sodium sulfate. The solvent was distilled off at reduced pressure to give 6.08 g of the titled compound (yield 99%).

¹H-NMR (DMSO-d₆) δ: 3.96 (3H, s), 7.06 (1H, d, J=8.0 Hz), 7.41 (1H, t, J=7.8 Hz), 7.58 (1H, t, J=8.0 Hz), 8.07 (1H, s).

### Reference Example 428

### (3-Fluoro-7-methoxy-1-benzothiophen-2-yl)methanol

n-Butyllithium (54.7 mL, 87.60 mmol) was added dropwise over a period of 20 min at −78° C. to a THF solution (200 mL) of 7-methoxy-1-benzothiophene-2-carboxylic acid (6.08 g, 29.20 mmol) obtained in Reference Example 427. The reaction solution was stirred for 1 hour, and a THF (70 mL) solution of N-fluoro-N-(phenylsulfonyl)benzenesulfonamide (18.41 g, 58.40 mmol) was added dropwise over a period of 15 min. The reaction solution was stirred for 1 hour, 1 N hydrochloric acid (100 mL) was then added while cooled on ice, and the mixture was extracted with ethyl acetate. The extract was washed with saturated brine, and the solvent was distilled off at reduced pressure to give 3-fluoro-7-methoxy-1-benzothiophene-2-carboxylic acid crude product (28.56 g). Borane-THF (87.6 mL, 87.6 mmol) was added while cooled on ice to a THF (200 mL) solution of the resulting 3-fluoro-7-methoxy-1-benzothiophene-2-carboxylic acid crude product (28.56 g), and the reaction solution was stirred for 90 min at 60° C. The addition of water to the reaction solution was followed by extraction with ethyl acetate. The extract was washed with saturated brine and then dried over sodium sulfate. The solvent was distilled off at reduced pressure, and the residue was purified by silica gel column chromatography (hexane/ethyl acetate: 85/15→67/33) to give the titled compound. Yield: 60%.

¹H-NMR (CDCl₃) δ: 1.81 (1H, t, J=6.2 Hz), 4.00 (3H, s), 4.92 (2H, dd, J=6.3, 1.4 Hz), 6.81 (1H, dd, J=6.3, 2.5 Hz), 7.30-7.40 (2H, m).

### Reference Example 429

### 3-Fluoro-7-methoxy-2-[3-(trifluoromethyl)benzyl]-1-benzothiophene

Phosphorus tribromide (1.72 mL, 18.26 mmol) was added while cooled on ice to a diethyl ether (80 mL)/THF (40 mL) solution of (3-fluoro-7-methoxy-1-benzothiophen-2-yl)methanol (3.69 g, 17.39 mmol) obtained in Reference Example 428, and the mixture was stirred for 3.5 hours. The addition of water to the reaction solution was followed by extraction with ethyl acetate. The extract was washed with saturated brine and then dried over sodium sulfate. The solvent was distilled off at reduced pressure to give 2-(bromomethyl)-3-fluoro-7-methoxy-1-benzothiophene crude product (6.56 g). A 2 M sodium carbonate aqueous solution (35 mL)-1,2-dimethoxyethane (175 mL) mixture of the resulting 2-(bromomethyl)-3-fluoro-7-methoxy-1-benzothiophene crude product (6.56 g), [3-(trifluoromethyl)phenyl]boronic acid (3.96 g, 20.87 mmol), and tetrakistriphenylphosphine palladium (0) (804 mg, 0.70 mmol) was heated to reflux over night in a nitrogen atmosphere. The reaction solution was diluted with saturated brine and ethyl acetate, and was filtered using celite. The resulting filtrate was extracted with ethyl acetate. The combined organic layers were dried over anhydrous sodium sulfate and then concentrated at reduced pressure, and the residue was purified by silica gel column chromatography (hexane-ethyl acetate) to give the titled compound. Yield: 41%. White solids.

¹H-NMR (CDCl₃) δ: 3.96 (3H, s), 4.24 (2H, s), 6.73-6.83 (1H, m), 7.29-7.36 (2H, m), 7.36-7.53 (3H, m), 7.55 (1H, s).

### Reference Example 430

### 3-Fluoro-2-[3-(trifluoromethyl)benzyl]-1-benzothiophen-7-yl trifluoromethanesulfonate

Boron tribromide-methylene chloride solution (19.5 mL, 19.5 mmol) was added while cooled on ice to a toluene (30 mL) solution of 3-fluoro-7-methoxy-2-[3-(trifluoromethyl)benzyl]-1-benzothiophene (1.66 g, 4.88 mmol) obtained in Reference Example 429, and the reaction solution was stirred for 4 hours at room temperature. The addition of water to the reaction solution was followed by extraction with ethyl acetate. The extract was washed with saturated brine and then dried over sodium sulfate. The solvent was distilled off at reduced pressure to give 3-fluoro-2-[3-(trifluoromethyl)benzyl]-1-benzothiophen-7-ol crude product. Trifluoromethanesulfonic anhydride (1.64 mL, 9.76 mmol) was added while cooled on ice to a pyridine (8 mL) solution of the resulting 3-fluoro-2-[3-(trifluoromethyl)benzyl]-1-benzothiophen-7-ol crude product, and the mixture was stirred for 1 hour. The reaction solution was added to 6 N hydrochloric acid cooled on ice, and was extracted with ethyl acetate. The extract was washed with saturated brine and then dried over sodium sulfate. The solvent was distilled off at reduced pressure, and the residue was purified by silica gel column chromatography (hexane/ethyl acetate: 97/3) to give the titled compound. Yield: 88%.

¹H-NMR (CDCl₃) δ: 4.27 (2H, s), 7.33 (1H, dd, J=8.0, 0.8 Hz), 7.38-7.63 (5H, m), 7.73 (1H, dd, J=8.0, 0.8 Hz).

### Reference Example 431

### Ethyl 3-[3-fluoro-2-[3-(trifluoromethyl)benzyl]-1-benzothiophen-7-yl]benzoate

3-Fluoro-2-[3-(trifluoromethyl)benzyl]-1-benzothiophene-7-yl trifluoromethanesulfonate obtained in Reference Example 430, and [3-(ethoxycarbonyl)phenyl]boronic acid were used in the same manner as in Reference Example 10 to obtain the titled compound. Yield: 42%. Oily substance.

¹H-NMR (CDCl₃) δ: 1.39 (3H, t, J=7.1 Hz), 4.24 (2H, s), 4.39 (2H, q, J=7.0 Hz), 7.34-7.66 (7H, m), 7.73 (1H, dd, J=7.8, 1.2 Hz), 7.79-7.86 (1H, m), 8.08 (1H, dt, J=1.4 Hz), 8.29 (1H, t, J=1.8 Hz).

### Reference Example 432

### Ethyl 3-fluoro-5-[3-fluoro-2-[3-(trifluoromethyl)benzyl]-1-benzothiophen-7-yl]benzoate

3-Fluoro-2-[3-(trifluoromethyl)benzyl]-1-benzothiophene-7-yl trifluoromethanesulfonate obtained in Reference Example 430, and ethyl 3-fluoro-5-(4,4,5,5-tetramethyl-1,3,2-dioxaborolane-2-yl)benzoate in synthesized Reference Example 225 were used in the same manner as in Reference Example 10 to obtain the titled compound. Yield: 38%. Oily substance.

¹H-NMR (CDCl₃) δ: 1.39 (3H, t, J=7.1 Hz), 4.25 (2H, s), 4.40 (2H, q, J=7.0 Hz), 7.36-7.59 (7H, m), 7.73-7.79 (2H, m), 8.11 (1H, t, J=1.5 Hz).

### Reference Example 433

### 5-Bromoimidazo[1,2-a]pyridine-2-carbaldehyde

1,1,3-Trichloropropan-2-one (316 μL, 3.0 mmol) was added at room temperature to a 1,2-dimethoxyethane (5.0 mL) solution of 6-bromopyridin-2-amine (173 mg, 1.0 mol), and the mixture was heated and stirred for 24 hours at 70° C. The reaction solution was concentrated at reduced pressure, the residue was dissolved in ethanol (5.0 mL), and the mixture was heated to reflux for 4 hours. The reaction solution was concentrated at reduced pressure, saturated calcium carbonate aqueous solution (5.0 mL) was added to the residue, and the mixture was heated to reflux for 90 min. The reaction solution was returned to room temperature, and was extracted with ethyl acetate, THF, and methylene chloride. The aqueous layer was made basic with 1 N sodium hydroxide aqueous solution, and was extracted with ethyl acetate. The extract was combined, washed with saturated sodium bicarbonate aqueous solution, and dried over sodium sulfate. The solvent was distilled off at reduced pressure, and the residue was purified by silica gel column chromatography (hexane/ethyl acetate: 80/20→30/70) to give the titled compound. Yield: 46%.

¹H-NMR (CDCl₃) δ: 7.15-7.24 (2H, m), 7.70 (1H, d, J=8.8 Hz), 8.36-8.40 (1H, m), 10.17 (1H, s).

### Reference Example 434

### (5-Bromoimidazo[1,2-a]pyridin-2-yl)[3-(trifluoromethyl)phenyl]methanol

A THF (10 mL) solution of 1-bromo-3-(trifluoromethyl)benzene (1.62 mL, 11.55 mmol) was added while cooled on ice to a THF (15 mL) solution of magnesium (365 mg, 15.02 mmol) and small pieces of iodine. The reaction solution was returned to room temperature, and a THF (35 mL) solution of 5-bromoimidazo[1,2-a]pyridine-2-carbaldehyde (1.30 g, 5.78 mmol) synthesized in Reference Example 433 was gradually added dropwise. The reaction solution was stirred for 3.5 hours at room temperature, saturated ammonium chloride aqueous solution was then added, and the mixture was extracted with ethyl acetate. The extract was washed with saturated brine and then dried over sodium sulfate. The solvent was distilled off at reduced pressure, and the residue was purified by silica gel column chromatography (hexane/ethyl acetate: 40/60→15/85) to give the titled compound. Yield: 63%. Oily substance.

¹H-NMR (CDCl₃) δ: 3.73 (1H, br s), 6.07 (1H, s), 7.02-7.22 (2H, m), 7.44-7.60 (4H, m), 7.70 (1H, d, J=7.1 Hz), 7.80 (1H, s).

### Reference Example 435

### Ethyl 3-[2-(hydroxymethyl)imidazo[1,2-a]pyridin-5-yl]benzoate

In the same manner as in Reference Example 433, 5-bromoimidazo[1,2-a]pyridine-2-carbaldehyde crude product (2.35 g) was synthesized from 6-bromopyridin-2-amine (5.0 g, 28.9 mmol), and sodium borohydride (42 mg, 1.1 mmol) was added while cooled on ice to a methanol (5 mL) solution of the resulting 5-bromoimidazo[1,2-a]pyridine-2-carbaldehyde crude product (225 mg). The reaction solution was stirred for 15 min, and the addition of water to the reaction solution was followed by extraction with ethyl acetate. The extract was dried over anhydrous sodium sulfate and was filtered off. The solvent was distilled off at reduced pressure to give (5-bromoimidazo[1,2-a]pyridin-2-yl)methanol crude product (211 mg). A mixture of the resulting (5-bromoimidazo[1,2-a]pyridin-2-yl)methanol crude product, [3-(ethoxycarbonyl)phenyl]boronic acid (216 mg, 1.12 mmol), tetrakistriphenylphosphine palladium (0) (43 mg, 0.037 mmol), and 2 M sodium carbonate aqueous solution (1.9 mL)-1,2-dimethoxyethane (9.0 mL) was heated to reflux over night in a nitrogen atmosphere. The reaction solution was diluted with saturated brine and ethyl acetate, and was filtered using celite. The resulting filtrate was extracted with ethyl acetate. The combined organic layers were dried over anhydrous sodium sulfate and then concentrated at reduced pressure, and the residue was purified by silica gel column chromatography (ethyl acetate-100:0→90:10) to give 185 mg of the titled compound (yield 23%).

¹H-NMR (CDCl₃) δ: 1.43 (3H, t, J=7.1 Hz), 1.61 (1H, br s), 4.43 (2H, q, J=7.1 Hz), 4.83 (2H, s), 6.78 (1H, dd, J=7.1, 1.4 Hz), 7.29 (1H, dd, J=9.1, 6.9 Hz), 7.55-7.68 (2H, m), 7.79-7.84 (1H, m), 8.18-8.23 (1H, m), 8.27-8.30 (1H, m).

### Reference Example 436

### Ethyl 3-[2-(bromomethyl)imidazo[1,2-a]pyridine-5-yl]benzoate

Ethyl 3-[2-(hydroxymethyl)imidazo[1,2-a]pyridine-5-yl]benzoate obtained in Reference Example 435, and phosphorus tribromide were used in the same manner as in Reference Example 179 to obtain the titled compound. Yield: 48%. White solid.

¹H-NMR (CDCl₃) δ: 1.42 (3H, t, J=7.1 Hz), 4.43 (2H, q, J=7.1 Hz), 4.62 (2H, d, J=0.5 Hz), 6.78 (1H, dd, J=6.9, 1.1 Hz), 7.30 (1H, dd, J=9.1, 6.9 Hz), 7.59-7.63 (2H, m), 7.63-7.68 (1H, m), 7.77-7.82 (1H, m), 8.18-8.23 (1H, m), 8.26-8.28 (1H, m).

### Reference Example 437

### Ethyl 3-[2-[3-(trifluoromethyl)benzyl]imidazo[1,2-a]pyridine-5-yl]benzoate

Ethyl 3-[2-(bromomethyl)imidazo[1,2-a]pyridine-5-yl]benzoate obtained in Reference Example 436, and [3-(trifluoromethyl)phenyl]boronic acid were used in the same manner as in Reference Example 200 to obtain the titled compound. Yield: 42%. Oily substance.

¹H-NMR (CDCl₃) δ: 1.40 (3H, t, J=7.1 Hz), 4.16 (2H, s), 4.41 (2H, q, J=7.1 Hz), 6.74 (1H, dd, J=6.9, 1.1 Hz), 7.21-7.30 (1H, m), 7.31-7.67 (7H, m), 7.76-7.81 (1H, m), 8.15-8.21 (1H, m), 8.25-8.30 (1H, m).

### Reference Example 438

### 2-Chloro-3-(methoxymethoxy)pyridine

Sodium hydride (1.7 g, 42.5 mmol) was added while cooled on ice to a THF (85 mL) solution of 2-chloropyridin-3-ol (5.0 g, 38.6 mmol). The mixture was stirred for 15 min, and chloromethyl methyl ether (3.22 mL, 42.5 mmol) was then added. The mixture was stirred for 30 min while cooled on ice, and was then stirred for 2 hours at room temperature. The addition of water to the reaction solution was followed by extraction with ethyl acetate. The extract was washed with saturated brine and then dried over sodium sulfate. The solvent was distilled off at reduced pressure, and the residue was purified by silica gel column chromatography (hexane/ethyl acetate: 90/10→67/33) to give the titled compound. Yield: 75%. White solids.

¹H-NMR (CDCl₃) δ: 3.52 (3H, s), 5.27 (2H, s), 7.18 (1H, dd, J=8.2, 4.7 Hz), 7.48 (1H, dd, J=8.2, 1.6 Hz), 8.05 (1H, dd, J=4.7, 1.6 Hz).

### Reference Example 439

### 2-Chloro-4-iodo-3-(methoxymethoxy)pyridine

n-Butyllithium (29.1 mL, 46.6 mmol) was added at −78° C. in a nitrogen atmosphere to a THF (200 mL) solution of 2-chloro-3-(methoxymethoxy)pyridine (5.39 g, 31.0 mmol) obtained in Reference Example 438. The reaction solution was stirred for 1 hour, and a THF (110 mL) solution of iodine (8.26 g, 32.6 mmol) was then added, and the mixture was stirred for 3 hours. The addition of saturated ammonium chloride aqueous solution to the reaction solution was followed by extraction with ethyl acetate. The extract was washed with saturated sodium thiosulfate aqueous solution and saturated brine, and was then dried over sodium sulfate. The solvent was distilled off at reduced pressure, and the residue was purified by silica gel column chromatography (hexane/ethyl acetate: 90/10→70/30) to give the titled compound. Yield: 81%. White solids.

¹H-NMR (CDCl₃) δ: 3.73 (3H, s), 5.22 (2H, s), 7.68 (1H, dd, J=4.9, 0.8 Hz), 7.79 (1H, dd, J=4.9, 0.8 Hz).

### Reference Example 440

### 2-Chloro-4-iodopyridin-3-ol

Hydrochloric acid-methanol (1.0 mL) was added to a methanol (20 mL) solution of 2-chloro-4-iodo-3-(methoxymethoxy)pyridine (500 mg, 1.67 mmol) obtained in Reference Example 439, and the mixture was stirred for 1.5 hours at 50° C. Excess triethylamine was added to the reaction solution, and the reaction solution was concentrated. The residue was purified by silica gel column chromatography (hexane/ethyl acetate: 90/10→67/33, ethyl acetate/methanol: 67/33) to give the titled compound. Yield: quant. White solids.

¹H-NMR (CDCl₃) δ: 5.91 (1H, s), 7.59-7.65 (1H, m).

### Reference Example 441

### (7-Chlorofuro[2,3-c]-pyridin-2-yl)methanol

An DMF (17 mL) solution of 2-chloro-4-iodopyridin-3-ol (430 mg, 1.68 mmol) obtained in Reference Example 440, tetrakistriphenylphosphine palladium (0) (194 mg, 0.168 mmol), copper iodide (I) (32 mg, 0.168 mmol), propargyl alcohol (139 μL, 2.36 mmol), and triethylamine (704 μL, 5.05 mmol) was stirred for 15 hours at 50° C. The addition of water to the reaction solution was followed by filtration with celite. The resulting filtrate was extracted with ethyl acetate. The extract was washed with saturated brine and dried over sodium sulfate. The solvent was distilled off at reduced pressure, and the residue was purified by silica gel column chromatography (hexane-ethyl acetate 80:20→50:50) to give 181 mg of the titled compound (yield 59%).

¹H-NMR (CDCl₃) δ: 4.35 (1H, d, J=6.0 Hz), 4.87 (2H, d, J=5.8 Hz), 6.78 (1H, s), 7.44 (1H, d, J=5.2 Hz), 8.18 (1H, d, J=5.2 Hz).

### Reference Example 442

### 7-Chlorofuro[2,3-c]-pyridine-2-carbaldehyde

(7-Chlorofuro[2,3-c]-pyridin-2-yl)methanol (180 mg, 0.98 mmol) obtained in Reference Example 441, 4A molecular sieve (900 mg), 4-methylmorpholine N-oxide (287 mg, 2.45 mmol), and tetra-n-propylammonium perruthenate (VII) (17.2 mg, 0.049 mmol) were dissolved in acetonitrile (9 mL), and the mixture was stirred for 3 days at room temperature. 2-Propanol was added, the mixture was filtered using celite, and the solvent was distilled off at reduced pressure. The residue was purified by silica gel column chromatography (hexane/ethyl acetate 67/33→50/50) to give the titled compound. Yield: 22%. White solids.

¹H-NMR (CDCl₃) δ: 7.58 (1H, s), 7.63 (1H, d, J=5.5 Hz), 8.32 (1H, d, J=5.5 Hz), 10.03 (1H, s).

### Reference Example 443

### (7-Chlorofuro[2,3-c]pyridin-2-yl)[3-(trifluoromethyl)phenyl]methanol

7-Chlorofuro[2,3-c]pyridine-2-carbaldehyde obtained in Reference Example 442, and 1-bromo-3-(trifluoromethyl)benzene were used in the same manner as in Reference Example 434 to synthesize the titled compound. Yield: 40%. Oily substance.

¹H-NMR (CDCl₃) δ: 2.96 (1H, br s), 6.09 (1H, d, J=3.8 Hz), 6.64-6.66 (1H, m), 7.42 (1H, d, J=5.2 Hz), 7.51-7.60 (1H, m), 7.62-7.75 (2H, m), 7.81 (1H, s), 8.18 (1H, d, J=5.2

### Reference Example 444

### Ethyl 3-[2-(hydroxymethyl)furo[2,3-c]pyridin-7-yl]benzoate

(7-Chlorofuro[2,3-c]pyridin-2-yl)methanol obtained in Reference Example 441, and [3-(ethoxycarbonyl)phenyl]boronic acid were used in the same manner as in Reference Example 10 to obtain the titled compound. Yield: 76%. Pale yellow solid.

¹H-NMR (CDCl₃) δ: 1.42 (3H, t, J=7.1 Hz), 2.89 (1H, br s), 4.41 (2H, q, J=7.1 Hz), 4.48 (2H, d, J=5.8 Hz), 6.74 (1H, s), 7.42-7.48 (1H, m), 7.52-7.61 (1H, m), 8.06-8.12 (1H, m), 8.45-8.53 (2H, m), 8.99 (1H, d, J=1.1 Hz).

### Reference Example 445

### Ethyl 3-[2-(bromomethyl)furo[2,3-c]pyridin-7-yl]benzoate

Ethyl 3-[2-(hydroxymethyl)furo[2,3-c]pyridin-7-yl]benzoate obtained in Reference Example 444, and phosphorus tribromide were used in the same manner as in Reference Example 179 to obtain the titled compound. Yield: 59%. White solid.

¹H-NMR (CDCl₃) δ: 1.45 (3H, t, J=7.1 Hz), 4.44 (2H, q, J=7.1 Hz), 4.65 (2H, s), 6.85 (1H, d, J=0.5 Hz), 7.48 (1H, d, J=4.9 Hz), 7.59-7.67 (1H, m), 8.13-8.18 (1H, m), 8.51-8.59 (2H, m), 9.03-9.09 (1H, m).

### Reference Example 446

### Ethyl 3-[2-[3-(trifluoromethyl)benzyl]furo[2,3-c]pyridin-7-yl]benzoate

Ethyl 3-[2-(bromomethyl)furo[2,3-c]pyridin-7-yl]benzoate obtained in Reference Example 445, and [3-(trifluoromethyl)phenyl]boronic acid were used in the same manner as in Reference Example 200 to obtain the titled compound. Yield: 41%. White solid.

¹H-NMR (CDCl₃) δ: 1.42 (3H, t, J=6.9 Hz), 4.28 (2H, s), 4.43 (2H, q, J=7.2 Hz), 6.48 (1H, s), 7.41 (1H, d, J=4.9 Hz), 7.46-7.67 (5H, m), 8.10-8.15 (1H, m), 8.46-8.53 (2H, m), 9.03 (1H, t, J=1.6 Hz).

### Reference Example 447

### 3,5-Diiodopyridine-4-ol

N-lodosuccinimide (99.0 g, 442 mmol) was added to pyridine-4-ol (20.0 g, 210 mmol) in methanol (700 mL), and the mixture was heated to reflux for 17 hours. The reaction solution was allowed to cool to room temperature. The precipitated crystals were filtered off. The residue was washed with methanol (200 mL) and hexane (100 mL), and dried to give the titled compound (70.0 g, 96%). White solid.

¹H-NMR (DMSO-d₆): δ 8.29 (2H, s), 11.98 (1H, br s).

### Reference Example 448

### Ethyl 3-[2-(hydroxymethyl)furo[3,2-c]pyridin-7-yl]benzoate

Tetrakistriphenylphosphine palladium (0) (11.7 g, 10.1 mmol) and copper iodide (1) (3.84 g, 20.2 mmol) were added to a pyrrolidine (404 mL) solution of 3,5-diiodopyridin-4-ol (70.0 g, 202 mmol) obtained in Reference Example 447. The reaction solution was stirred for 30 min at room temperature, propargyl alcohol (5.82 mL, 101 mmol) was gradually added dropwise over a period of 3 hours, and the mixture was then stirred for 4 days at room temperature. The reaction solution was concentrated at reduced pressure, the residue was dissolved in THF (400 mL), and the mixture was heated to reflux for 5 hours in a nitrogen atmosphere. The reaction solution was diluted with ethyl acetate and washed with water. The organic layer was dried over sodium sulfate. The solvent was distilled off at reduced pressure, and the residue was purified by silica gel column chromatography (hexane-ethyl acetate 50:50) to give (7-iodofuro[3,2-c]pyridin-2-yl)methanol crude product (6.00 g). A water (24.2 mL)-THF (48.5 mL) solution of the resulting (7-iodofuro[3,2-c]pyridin-2-yl)methanol crude product (6.00 g), [3-(ethoxycarbonyl)phenyl]boronic acid (4.23 g, 21.8 mmol), potassium phosphate (11.6 g, 54.5 mmol), dicyclohexyl[2′,4′,6′-tris(1-methylethyl)biphenyl-2-yl]phosphine (1.04 g, 2.18 mmol), and palladium acetate (11) (490 mg, 2.18 mmol) was stirred for 17 hours at 45° C. in a nitrogen atmosphere. The reaction solution was diluted with dichloromethane, and was washed with water and saturated brine. The organic layer was dried over sodium sulfate. The solvent was distilled off at reduced pressure, and the residue was purified by silica gel column chromatography (ethyl acetate) to give 2.74 g of the titled compound (yield 4.6%).

¹H-NMR (CDCl₃): δ 1.43 (3H, t, J=7.0 Hz), 2.22 (1H, br s), 4.43 (2H, q, J=7.2 Hz), 4.86 (2H, s), 6.84 (1H, s), 7.61 (1H, t, J=7.8 Hz), 8.03-8.05 (1H, m), 8.11-8.13 (1H, m), 8.51 (1H, dd, J=1.6, 1.6 Hz), 8.68 (1H, s), 8.88 (1H, s).

### Reference Example 449

### Ethyl 3-[2-[3-(trifluoromethyl)benzyl]furo[3,2-c]pyridin-7-yl]benzoate

Thionyl chloride (823 μL, 11.1 mmol) was gradually added dropwise while cooled on ice to a dichloromethane (30.7 mL) solution of ethyl 3-[2-(hydroxymethyl)furo[3,2-c]pyridin-7-yl]benzoate (2.74 g, 9.22 mmol) obtained in Reference Example 448. The mixture was stirred for 12 hours at room temperature, and the reaction solution was concentrated at reduced pressure to give ethyl 3-[2-(chloromethyl)furo[3,2-c]pyridin-7-yl]benzoate crude product (2.91 g). A water (5.0 mL)-THF (25.0 mL) solution of the resulting ethyl 3-[2-(chloromethyl)furo[3,2-c]pyridin-7-yl]benzoate crude product (2.91 g. 9.22 mmol), [3-(trifluoromethyl)phenyl]boronic acid (2.10 g, 11.1 mmol), cesium phosphate (9.01 g, 27.6 mmol), and [1,1′-bis(diphenylphosphino)ferrocene]palladium (II) dichloride-dichloromethane complex (1:1) (376 mg, 0.461 mmol) was stirred for 17 hours at 50° C. in a nitrogen atmosphere. The reaction solution was diluted with dichloromethane, and was washed with water and saturated brine. The organic layer was dried over magnesium sulfate. The solvent was distilled off at reduced pressure, and the residue was purified by silica gel column chromatography (hexane-ethyl acetate 67:33) to give 2.29 g of the titled compound (yield 58%).

¹H-NMR (CDCl₃): δ 1.42 (3H, t, J=7.2 Hz), 4.23 (2H, s), 4.43 (2H, q, J=7.2 Hz), 6.53 (1H, dd, J=0.8, 0.8 Hz), 7.47-7.62 (5H, m), 7.99-8.02 (1H, m), 8.11-8.13 (1H, m), 8.51 (1H, dd, J=2.0, 2.0 Hz), 8.66 (1H, s), 8.81 (1H, s).

### Reference Example 450

### 3-[2-[3-(Trifluoromethyl)benzyl]furo[3,2-c]pyridin-7-yl]benzoic acid

An ethanol (17.9 mL) mixed solution of ethyl 3-[2-[3-(trifluoromethyl)benzyl]furo[3,2-c]pyridin-7-yl]benzoate obtained in Reference Example 449 and 2 N sodium hydroxide aqueous solution (1.08 mL, 21.5 mmol) was stirred for 17 hours at 50° C. The reaction solution was returned to room temperature, and the reaction solution was neutralized using 10% citric acid aqueous solution. The precipitated solids were filtered off, and the residue was washed with water (50 mL) and hexane (50 mL), and was then dried to give the titled compound (1.94 g, 91%). White solids.

¹H-NMR (DMSO-d₆,): δ 4.39 (2H, s), 6.84 (1H, s), 7.59-7.68 (3H, m), 7.73 (1H, d, J=7.2 Hz), 7.78 (1H, br s), 8.02-8.04 (1H, m), 8.08-8.10 (1H, m), 8.45 (1H, dd, J=1.6, 1.6 Hz), 8.68 (1H, s), 8.87 (1H, s), 12.6 (1H, br s).

### Reference Example 451

### 2-Iodo-3-(methoxymethoxy)pyridine

2-Iodopyridine-3-ol, and chloromethylmethyl ether were used in the same manner as in Reference Example 438 to obtain the titled compound. Yield: 87%. Yellow solid.

¹H-NMR (CDCl₃): δ 3.52 (3H, s), 5.27 (2H, s), 7.18 (1H, dd, J=8.2, 4.6 Hz), 7.28 (1H, dd, J=8.0, 1.6 Hz), 8.06 (1H, dd, J=4.8, 1.6 Hz).

### Reference Example 452

### 4-Chloro-2-iodo-3-(methoxymethoxy)pyridine

n-Butyllithium (36.2 mL, 91.0 mmol) was added at −78° C. in a nitrogen atmosphere to a THF (150 mL) solution of 2-iodo-3-(methoxymethoxy)pyridine (20.0 g, 75.0 mmol) obtained in Reference Example 451. The mixture was stirred for 20 min, 2,2,6,6-tetramethylpiperidine (17 mL, 98.0 mmol) was then added, and the mixture was stirred for 4 hours. Hexachloroethane (21.9 g, 91.0 mmol) was added to the reaction solution. The reaction solution was warmed to room temperature, diluted with dichloromethane, and washed with saturated ammonium chloride aqueous solution and saturated brine, and the organic layer was dried over sodium sulfate. The solvent was distilled off at reduced pressure, and the residue was purified by silica gel column chromatography (hexane/ethyl acetate: 15/1) to give 12.4 g of the titled compound (yield 55%). Oily substance.

¹H NMR (CDCl₃): δ 3.73 (3H, s), 5.23 (2H, s), 7.29 (1H, d, J=4.0 Hz), 8.06 (1H, d, J=5.2 Hz).

### Reference Example 453

### 4-Chloro-2-iodopyridin-3-ol

Hydrochloric acid-methanol (82.8 mL, 166 mmol) was added at room temperature to a methanol (80 mL) solution of 4-chloro-2-iodo-3-(methoxymethoxy)pyridine (12.4 g, 41.4 mmol) obtained in Reference Example 452. The reaction solution was stirred for 17 hours at 50° C., and triethylamine (23 mL, 166 mmol) was then added while cooled on ice. The reaction solution was diluted with dichloromethane and washed with water and saturated brine, and the organic layer was then dried over sodium sulfate. The solvent was distilled off at reduced pressure, and the residue was purified by silica gel column chromatography (ethyl acetate) to give 9.54 g of the titled compound (yield 90%). Yellow solids.

¹H-NMR (CDCl₃): δ 7.42 (1H, d, J=5.2 Hz), 7.78 (1H, d, J=5.2 Hz), (1Hunconfirmed).

### Reference Example 454

### (7-Chlorofuro[3,2-b]-pyridin-2-yl)methanol

A triethylamine (124 mL) solution of 4-chloro-2-iodopyridin-3-ol (9.54 g, 37.3 mmol) obtained in Reference Example 453, bis(triphenylphosphine)palladium (II) dichloride (1.31 g, 1.87 mmol), copper iodide (1) (711 mg, 3.73 mmol), and propargyl alcohol (3.31 mL, 56.0 mmol) was stirred for 10 hours at 90° C. in a nitrogen atmosphere. The reaction solution was cooled to room temperature, the reaction solution was then diluted with dichloromethane and washed with water and saturated brine, and the organic layer was then dried over sodium sulfate. The solvent was distilled off at reduced pressure, and the residue was purified by silica gel column chromatography (hexane/ethyl acetate: 1/1) to give 3.40 g of the titled compound (yield 50%). Yellow solids.

¹H-NMR (CDCl₃): δ 4.89 (2H, s), 6.94 (1H, dd, J=0.8, 0.8 Hz), 7.25 (1H, d, J=5.2 Hz), 8.42 (1H, d, J=5.2 Hz), (1Hunconfirmed).

### Reference Example 455

### Ethyl 3-[2-(hydroxymethyl)furo[3,2-b]pyridin-7-yl]benzoate

A water (8.00 mL)-THF (16.0 mL) solution of (7-chlorofuro[3,2-b]-pyridin-2-yl)methanol (2.20 g, 12.0 mmol) obtained in Reference Example 454, [3-(ethoxycarbonyl)phenyl]boronic acid (3.49 g, 18.0 mmol), potassium phosphate (7.63 g, 35.9 mmol), dicyclohexyl[2′,4′,6′-tris(1-methylethyl)biphenyl-2-yl]phosphane (571 mg, 1.20 mmol), and palladium acetate (II) (2.76 g, 2.68 mmol) was stirred for 4 hours at 50° C. in a nitrogen atmosphere. The reaction solution was diluted with dichloromethane, and was washed with water and saturated brine. The organic layer was dried over sodium sulfate. The solvent was distilled off at reduced pressure, and the residue was purified by silica gel column chromatography (ethyl acetate) to give 2.48 g of the titled compound (yield 70%).

¹H-NMR (DMSO-d₆): δ 1.43 (3H, t, J=7.2 Hz), 2.43 (1H, br s), 4.43 (2H, q, J=7.2 Hz), 4.88 (2H, d, J=4.0 Hz), 6.97 (1H, dd, J=0.8, 0.8 Hz), 7.42 (1H, d, J=5.2 Hz), 7.62 (1H, t, J=8.0 Hz), 8.15 (2H, dd, J=8.0, 1.6 Hz), 8.58-8.60 (2H, m).

### Reference Example 456

### Ethyl 3-[2-(chloromethyl)furo[3,2-b]pyridin-7-yl]benzoate

Thionyl chloride (745 μL, 10.0 mmol) was gradually added dropwise at room temperature to a dichloromethane (28.0 mL) solution of ethyl 3-[2-(hydroxymethyl)furo[3,2-b]pyridin-7-yl]benzoate (2.48 g, 8.34 mmol) obtained in Reference Example 455. The reaction solution was stirred for 4 hours at room temperature, the reaction solution was then diluted with dichloromethane and washed with water and saturated brine, and the organic layer was then dried over sodium sulfate. The solvent was distilled off at reduced pressure, and the residue was purified by silica gel column chromatography (ethyl acetate) to give 2.28 g of the titled compound (yield 87%). Yellow solids.

¹H-NMR (DMSO-d₆): δ 1.35 (3H, t, J=7.2 Hz), 4.35 (2H, q, J=7.2 Hz), 5.07 (2H, s), 7.27 (1H, s), 7.70 (1H, d, J=5.2 Hz), 7.65 (1H, t, J=7.8 Hz), 8.09-8.11 (1H, m), 8.25-8.28 (1H, m), 8.59 (1H, dd, J=1.8, 1.8 Hz), 8.62 (1H, d, J=5.2 Hz).

### Reference Example 457

### Ethyl 3-[2-[3-(trifluoromethyl)benzyl]furo[3,2-b]pyridin-7-yl]benzoate

A water (6.0 mL)-THF (30.0 mL) solution of the ethyl 3-[2-(chloromethyl)furo[3,2-b]pyridin-7-yl]benzoate (2.28 g, 7.22 mmol) obtained in Reference Example 456, [3-(trifluoromethyl)phenyl]boronic acid (2.06 g, 10.8 mmol), cesium carbonate (7.06 g, 21.7 mmol), and [1,1′-bis(diphenylphosphino)ferrocene]palladium (II) dichloride-dichloromethane complex (1:1) (295 mg, 0.361 mmol) was stirred for 17 hours at 50° C. in a nitrogen atmosphere. The reaction solution was diluted with dichloromethane, and was washed with water and saturated brine. The organic layer was dried over magnesium sulfate. The solvent was distilled off at reduced pressure, and the residue was purified by silica gel column chromatography (hexane-ethyl acetate 1:1) to give 2.76 g of the titled compound (yield 90%).

¹H-NMR (DMSO-d₆): δ 1.42 (3H, t, J=7.2 Hz), 4.27 (2H, s), 4.43 (2H, q, J=7.2 Hz), 6.72 (1H, dd, J=0.8, 0.8 Hz), 7.40 (1H, d, J=4.8 Hz), 7.49 (1H, d, J=7.6 Hz), 7.54-7.57 (2H, m), 7.61 (1H, t, J=7.8 Hz), 7.64 (1H, br s), 8.09-8.16 (2H, m), 8.56-8.58 (2H, m).

### Reference Example 458

### 3-[2-[3-(Trifluoromethyl)benzyl]furo[3,2-b]pyridin-7-yl]benzoic acid

Ethyl 3-[2-[3-(trifluoromethyl)benzyl]furo[3,2-b]pyridin-7-yl]benzoate obtained in Reference Example 457, and sodium hydroxide aqueous solution were used in the same manner as in Reference Example 450 to synthesize the titled compound. Yield: 80%. White solid.

¹H-NMR (DMSO-d₆): ≢ 4.39 (2H, s), 6.89 (1H, s), 7.57-7.74 (5H, m), 7.78 (1H, s), 8.05 (1H, d, J=7.6 Hz), 8.17 (1H, d, J=7.6 Hz), 8.51-8.53 (2H, m), 13.27 (1H, br s).

### Reference Example 459

### 7-Bromo-2-[[3-(trifluoromethyl)phenoxy]methyl]-1-benzothiophene

(7-Bromo-1-benzothiophene-2-yl)methanol, and 3-(trifluoromethyl)phenol were used in the same manner as in Reference Example 420 to obtain the titled compound. Yield: 97%. Oily substance.

¹H-NMR (CDCl₃) δ: 5.36 (2H, s), 7.18 (1H, d, J=8.8 Hz), 7.21-7.30 (1H, m), 7.37-7.46 (2H, m), 7.49 (1H, d, J=7.7 Hz).

### Reference Example 460

### 4,4,5,5-tetramethyl-2-(2-[[3-(trifluoromethyl)phenoxy]methyl]-1-benzothiophen-7-yl)-1,3,2-dioxaborolane

7-Bromo-2-[[3-(trifluoromethyl)phenoxy]methyl]-1-benzothiophene obtained in Reference Example 459 was used in the same manner as in Reference Example 192 to obtain the titled compound. Yield: 61%. White solid.

¹H-NMR (CDCl₃) δ: 1.41 (12H, s), 5.36 (2H, d, J=1.1 Hz), 7.18 (1H, dd, J=8.2, 2.2 Hz), 7.21-7.29 (1H, m), 7.32-7.44 (3H, m), 7.83 (2H, ddd, J=9.8, 7.6, 1.4 Hz).

### Reference Example 461

### Ethyl 2-(2-[[3-(trifluoromethyl)phenoxy]methyl]-1-benzothiophen-7-yl)pyridine-4-carboxylate

Ethyl 2-bromopyridine-4-carboxylate, and 4,4,5,5-tetramethyl-2-(2-[[3-(trifluoromethyl)phenoxy]methyl]-1-benzothiophen-7-yl)-1,3,2-dioxaborolane synthesized in Reference Example 460 were used in the same manner as in Reference Example 4 to synthesize the titled compound. Yield: 55%. White solid.

¹H-NMR (CDCl₃) δ: 1.46 (3H, t, J=7.1 Hz), 4.48 (2H, q, J=7.1 Hz), 5.41 (2H, s), 7.16-7.25 (2H, m), 7.28 (1H, s), 7.36-7.45 (2H, m), 7.53 (1H, t, J=7.7 Hz), 7.83 (1H, dd, J=4.9, 1.4 Hz), 7.87 (1H, dd, J=7.8, 1.0 Hz), 8.01 (1H, dd, J=7.7, 1.1 Hz), 8.54 (1H, m, J=1.4 Hz), 8.96 (1H, dd, J=4.9, 0.8 Hz).

### Reference Example 462

### Ethyl 4-(2-[[3-(trifluoromethyl)phenoxy]methyl]-1-benzothiophen-7-yl)benzoate

Ethyl 4-bromobenzoate, and 4,4,5,5-tetramethyl-2-(2-[[3-(trifluoromethyl)phenoxy]methyl]-1-benzothiophen-7-yl)-1,3,2-dioxaborolane synthesized in Reference Example 460 were used in the same manner as in Reference Example 4 to synthesize the titled compound. Yield: 65%. White solid.

¹H-NMR (CDCl₃) δ: 1.43 (3H, t, J=7.1 Hz), 4.43 (2H, q, J=7.1 Hz), 5.36 (2H, s), 7.13-7.20 (1H, m), 7.21-7.26 (2H, m), 7.35-7.54 (4H, m), 7.75-7.84 (3H, m), 8.12-8.23 (2H, m).

### Reference Example 463

### N-(3-Bromophenyl)-3-methoxypropanamide

3-Methoxypropanoic acid, and 3-bromoaniline were used in the same manner as in Reference Example 134 to obtain the titled compound. Yield: 75%. Oily substance.

¹H-NMR (CDCl₃) δ: 2.59-2.68 (2H, m), 3.46 (3H, s), 3.69-3.76 (2H, m), 7.12-7.25 (2H, m), 7.43 (1H, dt, J=7.8, 1.6 Hz), 7.75 (1H, t, J=1.9 Hz), 8.29 (1H, br s).

### Reference Example 464

### N-[3-[2-(Bromomethyl)-1-benzothiophen-7-yl]phenyl]-3-methoxypropanamide

N-(3-Bromophenyl)-3-methoxypropanamide synthesized in Reference Example 463 is used in the same manner as in Reference Example 192 to obtain 3-methoxy-N-[3-(4,4,5,5-tetramethyl-1,3,2-dioxaborolane-2-yl)phenyl]propanamide crude product. The resulting crude product of 3-methoxy-N-[3-(4,4,5,5-tetramethyl-1,3,2-dioxaborolane-2-yl)phenyl]propanamide, and (7-bromo-1-benzothiophene-2-yl)methanol were used in the same manner as in Reference Example 4 to give N-[3-[2-(hydroxymethyl)-1-benzothiophene-7-yl]phenyl]-3-methoxypropanamide crude product. The resulting crude product of N-[3-[2-(hydroxymethyl)-1-benzothiophene-7-yl]phenyl]-3-methoxypropanamide, and phosphorus tribromide were used to obtain the titled compound. Yield: 13%. Oily substance.

¹H-NMR (CDCl₃) δ: 2.67 (2H, t, J=5.6 Hz), 3.46 (3H, s), 3.75 (2H, t, J=5.6 Hz), 4.78 (2H, s), 7.34-7.49 (5H, m), 7.58-7.66 (1H, m), 7.70 (1H, dd, J=7.7, 1.4 Hz), 7.80 (1H, s), 8.34 (1H, br s).

### Working Example 253

### N-(2-Hydroxyethyl)-3-[-methyl-2-[3-(trifluoromethyl)phenoxy]-1 H-benzimidazol-4-yl]benzamide

3-[1-Methyl-2-[3-(trifluoromethyl)phenoxy]-1 H-benzimidazole-4-yl]benzoic acid obtained in Reference Example 234, and 2-aminoethanol were used in the same manner as in Working Example 3 to obtain the titled compound. Yield: 46%, melting point: 139-140° C. (ethyl acetate-hexane).

¹H-NMR (CDCl₃) δ: 3.02 (1H, t, J=5.1 Hz), 3.54 (2H, q, J=5.1 Hz), 3.72 (2H, q, J=5.1 Hz), 3.77 (3H, s), 6.73 (1H, br s), 7.22-7.26 (1H, m), 7.32 (1H, t, J=7.5 Hz), 7.41-7.57 (4H, m), 7.70 (2H, d, J=7.8 Hz), 7.81 (1H, s), 8.09 (1H, dd, J=7.8, 1.2 Hz), 8.32 (1H, t, J=1.5 Hz).

### Working Example 254

### 3-[2-(3-Chloro-4-fluorobenzyl)-1-benzofuran-4-yl]-N-(2-methoxyethyl)benzamide

3-[2-(3-Chloro-4-fluorobenzyl)-1-benzofuran-4-yl]benzoic acid obtained in Reference Example 286, and 2-methoxyethanamine were used in the same manner as in Working Example 3 to obtain the titled compound. Yield: 62%, melting point: 77-78° C. (ethyl acetate-hexane).

¹H-NMR (CDCl₃) δ: 3.38 (3H, s), 3.58 (2H, q, J=4.8 Hz), 3.68 (2H, q, J=4.8 Hz), 4.07 (2H, s), 6.56 (1H, br s), 6.60 (1H, s), 7.07 (1H, t, J=8.4 Hz), 7.11-7.20 (1H, m), 7.25-7.37 (3H, m), 7.38-7.47 (1H, m), 7.53 (1H, t, J=7.8 Hz), 7.67-7.77 (2H, m), 8.01 (1H, t, J=1.5 Hz).

### Working Example 255

### 3-[2-(3,4-Difluorobenzyl)-1-benzofuran-4-yl]-N-(2-methoxyethyl)benzamide

3-[2-(3,4-difluorobenzyl)-1-benzofuran-4-yl]benzoic acid obtained in Reference Example 289, and 2-methoxyethanamine were used in the same manner as in Working Example 3 to obtain the titled compound. Yield: 59%, melting point: 83-84° C. (ethyl acetate-hexane). H-NMR (CDCl₃) δ: 3.38 (3H, s), 3.58 (2H, q, J=4.8 Hz), 3.68 (2H, q, J=4.8 Hz), 4.07 (2H, s), 6.50-6.69 (2H, m), 6.92-7.13 (3H, m), 7.26-7.34 (2H, m), 7.39-7.56 (1H, m), 7.52 (1H, t, J=7.5 Hz), 7.68-7.76 (2H, m), 8.00 (1H, t, J=1.5 Hz).

### Working Example 256

### N-(2-Cyanoethyl)-3-[2-(3-fluoro-4-methoxybenzyl)-1-benzofuran-7-yl]benzamide

3-[2-(3-Fluoro-4-methoxybenzyl)-1-benzofuran-7-yl]benzoic acid obtained in Reference Example 268, and 3-aminopropanenitrile were used in the same manner as in Working Example 3 to obtain the titled compound. Yield: 68%.

¹H-NMR (CDCl₃ ) δ: 2.77 (2H, t, J=6.2 Hz), 3.74 (2H, q, J=12.4, 6 Hz), 3.88 (3H, s), 4.07 (2H, s), 6.44 (1H, t, J=1.0 Hz), 6.54 (1H, s), 6.93 (1H, t, J=8.6 Hz), 7.03 (1H, d, J=9.6 Hz), 7.07 (1H, dd, J=11.8, 2.2 Hz), 7.28 (1H, t, J=7.2 Hz), 7.41 (1H, dd, J=7.6, 1.2 Hz), 7.49 (1H, dd, J=7.6, 1.2 Hz), 7.57 (1H, t, J=7.8 Hz), 7.78-7.81 (1H, m), 7.97-8.00 (1H, m), 8.21 (1H, t, J=1.6 Hz).

### Working Example 257

### 3-[2-(3-Chloro-4-fluorobenzyl)-1-benzofuran-7-yl]-N-(2-cyanoethyl)benzamide

3-[2-(3-Chloro-4-fluorobenzyl)-1-benzofuran-7-yl]benzoic acid obtained in Reference Example 272, and 3-aminopropanenitrile were used in the same manner as in Working Example 3 to obtain the titled compound. Yield: 82%.

¹H-NMR (CDCl₃) δ: 2.78 (2H, t, J=6.2 Hz), 3.75 (2H, q, J=12.2, 6.2 Hz), 4.10 (2H, s), 6.46 (1H, t, J=1.0 Hz), 6.58 (1H, brs), 7.11 (1H, t, J=8.6 Hz), 7.18-7.22 (1H, m), 7.30 (1H, t, J=7.4 Hz), 7.37 (1H, dd, J=7.0, 2.2 Hz), 7.42 (1H, dd, J=7.6, 1.2 Hz), 7.51 (1H, dd, J=7.6, 1.2 Hz), 7.58 (1H, td, J=7.7, 0.4 Hz), 7.78-7.80 (1H, m), 7.97-7.99 (1H, m), 8.22 (1H, t, J=1.4 Hz).

### Working Example 258

### 3-[2-(3-Chloro-4-fluorobenzyl)-1-benzofuran-7-yl]-N-(2-hydroxyethyl)benzamide

3-[2-(3-Chloro-4-fluorobenzyl)-1-benzofuran-7-yl]benzoic acid obtained in Reference Example 272, and 2-aminoethanol were used in the same manner as in Working Example 3 to obtain the titled compound. Yield: 78%.

¹H-NMR (CDCl₃) δ: 2.51 (1H, t, J=5.0 Hz), 3.67 (2H, dd, J=10.2, 5.4 Hz), 3.86 (2H, dd, J=9.8, 5.0 Hz), 4.09 (2H, s), 6.46 (1H, t, J=1.0 Hz), 6.58 (1H, brs), 7.11 (1H, t, J=8.6 Hz), 7.18-7.22 (1H, m), 7.29 (1H, t, J=7.4 Hz), 7.36 (1H, dd, J=7.2, 2.0 Hz), 7.42 (1H, dd, J=7.6, 1.2 Hz), 7.49 (1H, dd, J=7.6, 1.2 Hz), 7.56 (1H, t, J=7.6 Hz), 7.78-7.80 (1H, m), 7.94-7.97 (1H, m), 8.21 (1H, t, J=1.6 Hz).

### Working Example 259

### N-(2-Cyanoethyl)-3-[2-(3,4-difluorobenzyl)-1-benzofuran-7-yl]benzamide

3-[2-(3,4-Difluorobenzyl)-1-benzofuran-7-yl]benzoic acid obtained in Reference Example 276, and 3-aminopropanenitrile were used in the same manner as in Working Example 3 to obtain the titled compound. Yield: 82%.

¹H-NMR (CDCl₃) δ: 2.78 (2H, t, J=6.4 Hz), 3.75 (2H, q, J=11.2, 4.8 Hz), 4.10 (2H, s), 6.46 (1H, t, J=1.0 Hz), 6.59 (1H, br s), 7.02-7.06 (1H, m), 7.09-7.17 (2H, m), 7.29 (1H, t, J=8.2 Hz), 7.42 (1H, dd, J=7.6, 1.2 Hz), 7.50 (1H, dd, J=8.0, 1.2 Hz), 7.57 (1H, t, J=7.8 Hz), 7.77-7.80 (1H, m), 7.96-7.99 (1H, m), 8.22 (1H, t, J=1.4 Hz).

### Working Example 260

### 3-[2-(3,4-Difluorobenzyl)-1-benzofuran-7-yl]-N-(2-hydroxyethyl)benzamide

3-[2-(3,4-Difluorobenzyl)-1-benzofuran-7-yl]benzoic acid obtained in Reference Example 276, and 2-aminoethanol were used in the same manner as in Working Example 3 to obtain the titled compound. Yield: 97%.

¹H-NMR (CDCl₃) δ: 2.48 (1H, t, J=5.0 Hz), 3.67 (2H, dd, J=10.2, 5.6 Hz), 3.87 (2H, dd, J=10.0, 4.8 Hz), 4.09 (2H, s), 6.46 (1H, t, J=0.8 Hz), 6.61 (1H, br s), 7.04-7.09 (1H, m), 7.11-7.16 (2H, m), 7.29 (1H, t, J=7.2 Hz), 7.42 (1H, dd, J=7.6, 1.2 Hz), 7.49 (1H, dd, J=7.8, 1.4 Hz), 7.55 (1H, t, J=7.8 Hz), 7.78-7.80 (1H, m), 7.94-7.97 (1H, m), 8.22 (1H, t, J=1.4 Hz).

### Working Example 261

### 3-[2-(4-Chloro-3-fluorophenoxy)-1-methyl-1H-benzimidazol-4-yl]-N-(2-methoxyethyl)benzamide

3-[2-(4-Chloro-3-fluorophenoxy)-1-methyl-1H-benzimidazol-4-yl]benzoic acid obtained in Reference Example 291, and 2-methoxyethanamine were used in the same manner as in Working Example 3 to obtain the titled compound. Yield: 65%, melting point: 150-151° C. (ethyl acetate-hexane).

¹H-NMR (CDCl₃) δ: 3.35 (3H, s), 3.56 (2H, t, J=4.8 Hz), 3.67 (2H, q, J=4.8 Hz), 3.76 3H, s), 6.52 (1H, br s), 7.23-7.37 (1H, m), 7.40-7.54 (4H, m), 7.76 ((1H, d, J=7.8 Hz), 8.09 (1H, d, J=7.8 Hz), 8.38 (1H, s).

### Working Example 262

### N-(2-hydroxyethyl)-3-[2-[3-(trifluoromethyl)benzyl]-2,3-dihydro-1-benzofuran-4-yl]benzamide

3-[2-[3-(Trifluoromethyl)benzyl]-2,3-dihydro-1-benzofuran-4-yl]benzoic acid obtained in Reference Example 295, and 2-aminoethanol were used in the same manner as in Working Example 3 to obtain the titled compound. Yield: 59%, melting point: 120-121° C. (ethyl acetate-hexane).

¹H-NMR (CDCl₃) δ: 2.42 (1H, br s), 2.94-3.06 (2H, m), 3.17 (1H, dd, J=14.7, 7.5 Hz), 3.34 (1H, dd, J=15.3, 8.4 Hz), 3.65 (2H, q, J=5.1 Hz), 3.85 (2H, br s), 4.98-5.05 (1H, m), 6.62 (1H, br s), 6.80 (1H, d, J=8.1 Hz), 6.90 (1H, d, J=7.5 Hz), 7.20 (1H, t, J=8.1 Hz), 7.38-7.57 (6H, m), 7.72 (1H, d, J=7.2 Hz), 7.85 (1H, s).

### Working Example 263

### 3-[2-(3-Chloro-4-fluorobenzyl)-1-benzothiophen-7-yl]-N-(2-cyanoethyl)-5-fluorobenzamide

3-[2-(3-Chloro-4-fluorobenzyl)-1-benzothiophen-7-yl]-5-fluorobenzoic acid obtained in Reference Example 294, and 3-aminopropanenitrile were used in the same manner as in Working Example 3 to obtain the titled compound. Yield: 70%, melting point: 127-128° C. (ethyl acetate-hexane).

¹H-NMR (CDCl₃) δ: 2.75 (2H, t, J=6.3 Hz), 3.72 (2H, q, J=6.3 Hz), 4.16 (2H, s), 6.68 (1H, br s), 7.03-7.18 (3H, m), 7.20-7.35 (2H, m), 7.42 (1H, t, J=7.5 Hz), 7.50-7.57 (2H, m), 7.70 (1H, d, J=7.8 Hz), 7.83 (1H, s).

### Working Example 264

### 3-[2-(3-Chloro-4-fluorobenzyl)-1-benzothiophen-7-yl]-5-fluoro-N-(2-methoxyethyl)benzamide

3-[2-(3-Chloro-4-fluorobenzyl)-1-benzothiophen-7-yl]-5-fluorobenzoic acid obtained in Reference Example 294, and 2-methoxyethanamine were used in the same manner as in Working Example 3 to obtain the titled compound. Yield: 70%, melting point: 108-109° C. (ethyl acetate-hexane).

¹H-NMR (CDCl₃) δ: 3.37 (3H, s), 3.55 (2H, t, J=4.8 Hz), 3.66 (2H, q, J=4.8 Hz), 4.16 (2H, s), 6.53 (1H, br s), 7.02-7.20 (3H, m), 7.26-7.32 (2H, m), 7.42 (1H, t, J=7.5 Hz), 7.50-7.57 (2H, m), 7.70 (1H, d, J=8.1 Hz), 7.81 (1H, s).

### Working Example 265

### N-(2-hydroxyethyl)-3-[2-[3-(trifluoromethyl)benzyl]thieno[3,2-b]pyridin-7-yl]benzamide

3-[2-[3-(Trifluoromethyl)benzyl]thieno[3,2-b]pyridin-7-yl]benzoic acid obtained in Reference Example 264, and 2-aminoethanol were used in the same manner as in Working Example 3 to obtain the titled compound. Yield: 30%., amorphous solid.

¹H-NMR (CDCl₃) δ: 2.57 (1H, br s), 3.66 (2H, q, J=5.1 Hz), 3.86 (2H, t, J=5.1 Hz), 4.32 (2H, s), 6.73 (1H, br s), 7.20-7.27 (1H, m), 7.34 (1H, s), 7.41-7.62 (5H, m), 7.80-7.88 (2H, m), 8.10 (1H, s), 8.68 (1H, d, J=4.5 Hz).

### Working Example 266

### N-(2-Amino-2-oxoethyl)-3-[2-[3-(trifluoromethyl)benzyl]thieno[3,2-b]pyridin-7-yl]benzamide

3-[2-[3-(Trifluoromethyl)benzyl]thieno[3,2-b]pyridin-7-yl]benzoic acid obtained in Reference Example 264, and glycinamide hydrochloride were used in the same manner as in Working Example 196 to obtain the titled compound. Yield: 70% melting point: 183-184° C. (ethyl acetate-hexane).

¹H-NMR (CDCl₃) δ: 4.19 (2H, d, J=5.1 Hz), 4.32 (2H, s), 5.22 (1H, br s), 6.07 (1H, br s), 7.09 (1H, br s), 7.22-7.26 (1H, m), 7.35 (1H, s), 7.39-7.61 (5H, m), 7.81-7.91 (2H, m), 8.15 (1H, s), 8.68 (1H, d, J=4.8 Hz).

### Working Example 267

### 3-[2-[3-(Trifluoromethyl)benzyl]thieno[3,2-b]pyridin-7-yl]benzamide

3-[2-[3-(Trifluoromethyl)benzyl]thieno[3,2-b]pyridin-7-yl]benzoic acid obtained in Reference Example 264, and 28% aqueous ammonia were used in the same manner as in Working Example 197 to obtain the titled compound. Yield: 64% melting point: 191-192° C. (ethyl acetate-hexane).

¹H-NMR (CDCl₃) δ: 4.32 (2H, s), 5.72 (1H, br s), 6.18 (1H, br s), 7.21-7.27 (1H, m), 7.35 (1H, s), 7.39-7.65 (5H, m), 7.86 (1H, d, J=7.8 Hz), 7.89 (1H, d, J=7.8 Hz), 8.14 (1H, s), 8.69 (1H, d, J=4.8 Hz).

### Working Example 268

### N-(2-Amino-2-oxoethyl)-3-fluoro-5-[4-fluoro-2-[3-(trifluoromethyl)benzyl]-1-benzothiopen-7-yl]benzamide

3-Fluoro-5-[4-fluoro-2-[3-(trifluoromethyl)benzyl]-1-benzothiophen-7-yl]benzoic acid obtained in Reference Example 279, and glycinamide hydrochloride were used in the same manner as in Working Example 196 to obtain the titled compound. Yield: 86% melting point: 177-178° C. (ethyl acetate-hexane).

¹H-NMR (CDCl₃) δ: 4.17 (2H, d, J=5.1 Hz), 4.28 (2H, s), 5.48 (1H, br s), 5.94 (1H, br s), 6.98 (1H, br s), 7.09 (1H, t, J=8.7 Hz), 7.20-7.26 (3H, m), 7.41-7.54 (5H, m), 7.82 (1H, s).

### Working Example 269

### 3-Fluoro-5-[4-fluoro-2-[3-(trifluoromethyl)benzyl]-1-benzothiophen-7-yl]benzamide

3-Fluoro-5-[4-fluoro-2-[3-(trifluoromethyl)benzyl]-1-benzothiophen-7-yl]benzoic acid obtained in Reference Example 279, and 28% aqueous ammonia were used in the same manner as in Working Example 197 to obtain the titled compound. Yield: 53% melting point: 161-162° C. (ethyl acetate-hexane).

¹H-NMR (CDCl₃) δ: 4.28 (2H, s), 5.70 (1H, br s), 6.02 (1H, br s), 7.10 (1H, t, J=8.7 Hz), 7.21-7.30 (3H, m), 7.42-7.54 (5H, m), 7.81 (1H, d, J=1.5 Hz).

### Working Example 270

### N-(2-Amino-2-oxoethyl)-3-[2-(3-chloro-5-fluorobenzyl)-4-fluoro-1-benzothiophen-7-yl]-4-fluorobenzamide

3-[2-(3-Chloro-5-fluorobenzyl)-4-fluoro-1-benzothiophen-7-yl]-4-fluorobenzoic acid obtained in Reference Example 283, and glycinamide hydrochloride were used in the same manner as in Working Example 196 to obtain the titled compound. Yield: 71%, melting point: 178-179° C. (ethyl acetate-hexane).

¹H-NMR (CDCl₃) δ: 4.14-4.21 (4H, m), 5.47 (1H, br s), 5.95 (1H, br s), 6.83-7.00 (3H, m), 7.03-7.14 (2H, m), 7.19-7.28 (3H, m), 7.81-7.88 (1H, m), 7.96-8.03 (1H, m).

### Working Example 271

### 3-Fluoro-5-[4-fluoro-2-[3-(trifluoromethyl)benzyl]-1-benzothiophen-7-yl]-N-(2-hydroxyethyl)benzamide

3-Fluoro-5-[4-fluoro-2-[3-(trifluoromethyl)benzyl]-1-benzothiophen-7-yl]benzoic acid obtained in Reference Example 279, and 2-aminoethanol were used in the same manner as in Working Example 3 to obtain the titled compound. Yield: 23%, melting point: 107-108° C. (ethyl acetate-hexane).

¹H-NMR (CDCl₃) δ: 2.30 (1H, br s), 3.57-3.69 (2H, m), 3.73-3.90 (2H, m), 4.27 (2H, s), 6.62 (1H, br s), 7.09 (1H, dd, J=9.6, 8.4 Hz), 7.20-7.30 (2H, s), 7.43-7.53 (6H, m), 7.78 (1H, s).

### Working Example 272

### 3-[2-(3-Chloro-4-fluorobenzyl)-1-benzofuran-4-yl]-N-(2-cyanoethyl)benzamide

3-[2-(3-Chloro-4-fluorobenzyl)-1-benzofuran-4-yl]benzoic acid obtained in Reference Example 286, and 3-aminopropanenitrile were used in the same manner as in Working Example 3 to obtain the titled compound. Yield: 55% melting point: 130-131° C. (hexane-ethyl acetate)

¹H-NMR (CDCl₃) δ: 2.78 (2H, t, J=6.2 Hz), 3.75 (2H, q, J=6.2 Hz), 4.08 (2H, s), 6.60 (1H, s), 6.64 (1H, t, J=6.2 Hz), 7.08 (1H, t, J=8.5 Hz), 7.15 (1H, ddd, J=4.5, 2.1, 1.9 Hz), 7.27-7.37 (3H, m), 7.41-7.46 (1H, m), 7.52-7.60 (1H, m), 7.76 (2 H, dd, J=7.7, 1.6 Hz), 8.02 (1H, t, J=1.6 Hz).

### Working Example 273

### N-(2-Amino-2-oxoethyl)-3-[2-(3-chloro-4-fluorobenzyl)-1-benzofuran-4-yl]benzamide

3-[2-(3-Chloro-4-fluorobenzyl)-1-benzofuran-4-yl]benzoic acid obtained in Reference Example 286, and glycinamide hydrochloride were used in the same manner as in Working Example 12 to obtain the titled compound. Yield: 49% melting point: 155-156° C. (ethyl acetate-hexane)

¹H-NMR (CDCl₃) δ: 4.07 (2H, s), 4.21 (2H, d, J=5.2 Hz), 5.51 (1H, br s), 6.10 (1H, br s), 6.59 (1H, s), 7.00-7.12 (2H, m), 7.12-7.21 (1H, m), 7.27-7.37 (3H, m), 7.39-7.46 (1H, m), 7.55 (1H, t, J=7.7 Hz), 7.78 (2H, dddd, J=17.0,7.8, 1.5, 1.4 Hz), 8.06 (1H, t, J=1.6 Hz).

### Working Example 274

### N-(2-Amino-2-oxoethyl)-3-[2-(3-chloro-5-fluorobenzyl)-1-benzofuran-4-yl]benzamide

3-[2-(3-Chloro-5-fluorobenzyl)-1-benzofuran-4-yl]benzoic acid obtained in Reference Example 299, and glycinamide hydrochloride were used in the same manner as in Working Example 12 to obtain the titled compound. Yield: 67% melting point: 156-157° C. (ethyl acetate). ¹H-NMR (CDCl₃) δ: 4.09 (2H, s), 4.21 (2H, d, J=4.7 Hz), 5.48 (1H, br s), 5.99 (1H, br s), 6.63 (1H, s), 6.92 (1H, d, J=7.7 Hz), 6.98 (2H, dt, J=8.5, 2.1 Hz), 7.09 (1H, s), 7.29-7.38 (2H, m), 7.44 (1H, dd, J=6.5, 1.5 Hz), 7.56 (1H, t, J=7.7 Hz), 7.79 (2H, dd, J=15.0, 7.8 Hz), 8.07 (1H, t, J=1.8 Hz).

### Working Example 275

### tert-Butyl (3-[[(3-[2-[3-(trifluoromethyl)benzyl]-1-benzofuran-4-yl]phenyl)carbonyl]amino]propyl)carbamate

3-[2-[3-(Trifluoromethyl)benzyl]-1-benzofuran-4-yl]benzoic acid obtained in Reference Example 10, and tert-butyl (3-aminopropyl)carbamate were used in the same manner as in Working Example 3 to obtain the titled compound. Yield: 62%

¹H-NMR (CDCl₃) δ: 1.44 (9H, s), 1.67-1.81 (2H, m), 3.27 (2H, q, J=6.0 Hz), 3.47-3.62 (2H, m), 3.49-3.60 (2H, m), 4.18 (2H, s), 4.88 (1H, br s), 6.68 (1H, s), 7.29-7.61 (9H, m), 7.73 (1H, d, J=7.7 Hz), 7.84 (1H, d, J=7.4 Hz), 8.13 (1H, s).

### Working Example 276

### N-(3-Aminopropyl)-3-[2-[3-(trifluoromethyl)benzyl]-1-benzofuran-4-yl]benzamide monohydrochloride

4 N hydrogen chloride-ethyl acetate (3.1 mL, 12.4 mmol) was added to an ethyl acetate (8 mL) solution of tert-butyl (3-[[(3-[2-[3-(trifluoromethyl)benzyl]-1-benzofuran-4-yl]phenyl)carbonyl]amino]propyl)carbamate (0.34 g, 0.62 mmol) obtained in Working Example 275, and the mixture was stirred for 4 hours at room temperature. The reaction solution was diluted with ethyl acetate and then filtered off to give 0.18 g of the titled compound (yield 60%).

¹H-NMR (DMSO-d₆) δ: 1.76-1.94 (2H, m), 2.72-2.92 (2H, m), 3.36 (2H, q, J=6.2 Hz), 4.32 (2H, s), 6.87 (1H, s), 7.28-7.42 (2H, m), 7.46-7.67 (5H, m), 7.70 (1H, s), 7.78 (1H, d, J=7.7 Hz), 7.90 (1H, d, J=8.0 Hz), 7.98 (3H, br s), 8.09 (1H, s), 8.83 (1H, t, J=5.6 Hz).

### Working Example 277

### tert-Butyl (2-[[(3-[2-[3-(trifluoromethyl)benzyl]-1-benzofuran-4-yl]phenyl)carbonyl]amino]ethyl)carbamate

3-[2-[3-(Trifluoromethyl)benzyl]-1-benzofuran-4-yl]benzoic acid obtained in Reference Example 10, and tert-butyl (2-aminoethyl)carbamate were used in the same manner as in Working Example 3 to obtain the titled compound. Yield: 89%

¹H-NMR (CDCl₃) δ: 1.38 (9H, s), 3.37-3.49 (2H, m), 3.54-3.64 (2H, m), 4.18 (2H, s), 4.97 (1H, br s), 6.67 (1H, s), 7.27-7.35 (3H, m), 7.38-7.46 (2H, m), 7.46-7.56 (3H, m), 7.57 (1H, s), 7.72 (1H, dt, J=7.7, 1.5 Hz), 7.81 (1H, d, J=7.7 Hz), 8.08 (1H, s).

### Working Example 278

### N-(2-Aminoethyl)-3-[2-[3-(trifluoromethyl)benzyl]-1-benzofuran-4-yl]benzamide monohydrochloride

tert-Butyl (2-[[(3-[2-[3-(trifluoromethyl)benzyl]-1-benzofuran-4-yl]phenyl)carbonyl]amino]ethyl)carbamate obtained in Working Example 277 was used in the same manner as in Working Example 276 to obtain the titled compound. Yield: 62%

¹H-NMR (DMSO-d₆) δ: 3.00 (2H, t, J=6.0 Hz), 3.55 (2H, q, J=5.7 Hz), 4.32 (2H, s), 6.88 (1H, s), 7.26-7.45 (2H, m), 7.46-7.68 (5H, m), 7.70 (1H, s), 7.80 (1H, d, J=7.7 Hz), 7.93 (1H, d, J=8.0 Hz), 8.03 (3H, br s), 8.12 (1H, s), 8.84 (1H, t, J=5.4 Hz).

### Working Example 279

### N-[3-[(Methylsulfonyl)amino]propyl]-3-[2-[3-(trifluoromethyl)benzyl]-1-benzofuran-4-yl]benzamide

Methanesulfonyl chloride (34 μL, 0.44 mmol) was added to a THF (2 mL) solution of N-(3-aminopropyl)-3-[2-[3-(trifluoromethyl)benzyl]-1-benzofuran-4-yl]benzamide monohydrochloride (0.18 g, 0.37 mmol) obtained in Working Example 276 and triethylamine (0.10 μL, 0.74 mmol), and the mixture was stirred for 17 hours at room temperature. The reaction solution was made acidic with the addition of 1 N hydrochloric acid, water was then added, and the aqueous layer was extracted with ethyl acetate. The resulting extract was washed with saturated brine and dried over anhydrous magnesium sulfate, and the solvent was then distilled off at reduced pressure. The residue was purified by silica gel column chromatography (hexane-ethyl acetate=4:1→1:4) and was then recrystallized from hexane-ethyl acetate to give 79 mg of the titled compound (yield 40%). Melting point: 79-80° C. (ethyl acetate-hexane).

¹H-NMR (CDCl₃) δ: 1.78-1.94 (2H, m), 2.97 (3H, s), 3.18-3.30 (2H, m), 3.66 (2H, q, J=6.2 Hz), 4.19 (2H, s), 5.20 (1H, t, J=6.6 Hz), 6.64 (2H, s), 7.29-7.34 (2H, m), 7.40-7.60 (6H, m), 7.69-7.81 (2H, m), 8.03 (1H, s).

### Working Example 280

### 3-[2-(3-Chloro-5-fluorobenzyl)-1-benzofuran-4-yl]-N-(2-methoxyethyl)benzamide

3-[2-(3-Chloro-5-fluorobenzyl)-1-benzofuran-4-yl]benzoic acid obtained in Reference Example 299, and 2-methoxyethanamine were used in the same manner as in Working Example 3 to obtain the titled compound. Yield: 40%, oily substance.

¹H-NMR (CDCl₃) δ: 3.39 (3 H, s), 3.54-3.62 (2H, m), 3.69 (2H, t, J=5.2 Hz), 4.09 (2H, s), 6.58 (1H, brs), 6.65 (1H, s), 6.92 (1H, dd, J=9.1, 1.6 Hz), 6.98 (1H, dt, J=8.2, 2.1 Hz), 7.09 (1H, s), 7.28-7.37 (2H, m), 7.41-7.47 (1H, m), 7.54 (1H, t, J=7.7 Hz), 7.66-7.83 (2H, m), 8.03(1H, t, J=1.5 Hz).

### Working Example 281

### N-[2-[(Methylsulfonyl)amino]ethyl]-3-[2-[3-(trifluoromethyl)benzyl]-1-benzofuran-4-yl]benzamide

N-(2-Aminoethyl)-3-[2-[3-(trifluoromethyl)benzyl]-1-benzofuran-4-yl]benzamide monohydrochloride obtained in Working Example 278, and methanesulfonyl chloride were used in the same manner as in Working Example 279 to obtain the titled compound. Yield: 62%, amorphous solid.

¹H-NMR (CDCl₃) δ: 2.98 (3H, s), 3.39-3.48 (2H, m), 3.66 (2H, q, J=5.6 Hz), 4.19 (2H, s), 4.92 (1H, t, J=5.8 Hz), 6.66 (1H, s), 6.84 (1H, br s), 7.28-7.35 (2H, m), 7.38-7.47 (2H, m), 7.47-7.62 (4H, m), 7.76 (2H, dd, J=15.0, 7.8 Hz), 8.05 (1H, s).

### Working Example 282

### 3-[2-(3-Chloro-4-fluorobenzyl)-3-methyl-1-benzothiophen-7-yl]-N-(2-hydroxyethyl)benzamide

2 N sodium hydroxide aqueous solution (1.0 mL, 2.0 mmol) was added to an ethanol (5 mL)-THF (2 mL) solution of ethyl 3-[2-(3-chloro-4-fluorobenzyl)-3-methyl-1-benzothiophen-7-yl]benzoate (0.46 g, 1.0 mmol) obtained in Reference Example 300, and the mixture was stirred for 30 min at 60° C. The solvent was distilled off at reduced pressure, and the reaction solution was neutralized with 1 N hydrochloric acid and then extracted with ethyl acetate. The extract was washed with saturated brine and dried over anhydrous magnesium sulfate, and the solvent was then distilled off at reduced pressure to give 0.40 g of 3-[2-(3-chloro-4-fluorobenzyl)-3-methyl-1-benzothiophen-7-yl]benzoic acid crude product. A mixture of the resulting 3-[2-(3-chloro-4-fluorobenzyl)-3-methyl-1-benzothiophen-7-yl]benzoic acid crude product (0.21 g), 2-aminoethanol (37 μL, 0.61 mmol), WSC (0.12 g, 0.61 mmol), HOBt (83 mg, 0.61 mmol), and DMF (2 mL) was stirred for 16 hours at room temperature. The reaction solution was diluted with water and extracted with ethyl acetate. The extract was washed with saturated brine and dried over anhydrous magnesium sulfate, and the solvent was then distilled off at reduced pressure. The residue was purified by silica gel column chromatography (hexane:ethyl acetate=1:1→3:7), and the resulting crystals were recrystallized from hexane-ethyl acetate to give 0.13 g of the titled compound (yield 55%). Melting point: 146-147° C. (hexane-ethyl acetate).

¹H-NMR (CDCl₃) δ: 2.40 (3H, s), 2.45 (1H, t, J=5.1 Hz), 3.61-3.69 (2H, m), 3.85 (2H, q, J=5.1 Hz), 4.15 (2H, s), 6.62 (1H, br s), 6.98-7.12 (2H, m), 7.22 (1H, dd, J=7.0, 1.5 Hz), 7.35 (1H, dd, J=7.1, 1.1 Hz),7.44-7.59 (2H, m), 7.67 (1H, dd, J=8.0, 1.1 Hz), 7.77-7.86 (2H, m), 8.05 (1H, t, J=1.6 Hz).

### Working Example 283

### 3-[2-(3-Chloro-4-fluorobenzyl)-3-methyl-1-benzothiophen-7-yl]-N-(2-methoxyethyl)benzamide

Ethyl 3-[2-(3-chloro-4-fluorobenzyl)-3-methyl-1-benzothiophen-7-yl]benzoate obtained in Reference Example 300 was used in the same manner as in Working Example 282 to give 3-[2-(3-chloro-4-fluorobenzyl)-3-methyl-1-benzothiophene-7-yl]benzoic acid crude product. The crude product of 3-[2-(3-chloro-4-fluorobenzyl)-3-methyl-1-benzothiophene-7-yl]benzoic acid, and 2-methoxyethanamine were used to give the titled compound. Yield: 59% melting point: 141-142° C. (hexane-ethyl acetate).

¹H-NMR (CDCl₃) δ: 2.41 (3H, s), 3.37 (3H, s), 3.52-3.63 (2H, m), 3.62-3.73 (2H, m), 4.15(2H, s), 6.53(1H, br s), 6.95-7.11 (2H, m), 7.22(1H, dd, J=6.9, 1.9 Hz), 7.36 (1H, dd, J=7.4, 1.1 Hz), 7.45-7.59 (2H, m), 7.67 (1H, dd, J=8.0, 1.1 Hz), 7.76-7.87 (2H, m), 8.05 (1H, t, J=1.8 Hz).

### Working Example 284

### 3-[2-(3-Acetylbenzyl)-1-benzothiophen-7-yl]-N-(2-methoxyethyl)benzamide

Ethyl 3-[2-(3-acetylbenzyl)-1-benzothiophen-7-yl]benzoate obtained in Reference Example 301 wad used in the same manner as in Working. Example 282 was used to give 3-[2-(3-acetylbenzyl)-1-benzothiophene-7-yl]benzoic acid crude product. The resulting crude product of 3-[2-(3-acetylbenzyl)-1-benzothiophene-7-yl]benzoic acid, and 2-methoxyethanamine were used to give the titled compound. Yield: 64% melting point: 81-82° C. (hexane-ethyl acetate)

¹H-NMR (CDCl₃) δ: 2.59 (3H, s), 3.38 (3H, s), 3.52-3.61 (2H, m), 3.63-3.72 (2H, m), 4.28 (2H, s), 6.53 (1H, br s), 7.11 (1H, s), 7.28-7.34 (1H, m), 7.37-7.45 (2H, ma), 7.45-7.50(1H, m), 7.53 (1H, t, J=7.7 Hz), 7.68 (1H, dd, J=8.0, 1.1 Hz), 7.76-7.86 (3H, m), 7.88 (1H, s), 8.04 (1H, t, J=1.8 Hz).

### Working Example 285

### 3-[2-(3,4-Difluorobenzyl)-1-benzothiophen-4-yl]benzamide

2 N sodium hydroxide aqueous solution (0.99 mL, 1.98 mmol) was added to a THF (3 mL)-methanol (2 mL) solution of ethyl 3-[2-(3,4-difluorobenzyl)-1-benzothiophen-4-yl]benzoate (0.27 g, 0.66 mmol) obtained in Reference Example 207, and the mixture was stirred for 4 hours at room temperature. The reaction solution was made acidic with the addition of 1 N hydrochloric acid, and was extracted with ethyl acetate. The extract was washed with saturated brine and dried over anhydrous magnesium sulfate, and the solvent was then distilled off at reduced pressure. A mixture of the residue, WSC (0.15 g, 0.77 mmol), HOBt (0.10 g, 0.77 mmol), and DMF (3 mL) was stirred for 2 hours, the reaction solution was then added to 25% aqueous ammonia (5 mL), and the mixture was stirred for 15 min. The reaction solution was extracted with ethyl acetate. The extract was washed with water, washed with saturated brine, and dried over anhydrous magnesium sulfate, and the solvent was distilled off at reduced pressure. The residue was washed with ethyl acetate and recrystallized from ethyl acetate to give 0.12 g of the titled compound (yield 47%).

Melting point: 189-190° C. (ethyl acetate).

¹H NMR (CDCl₃) δ: 4.17 (2H, s), 5.63 (1H, br s), 6.09 (1H, br s), 6.93-7.01 (1H, m), 7.01-7.10 (2H, m), 7.12 (1H, s), 7.28-7.41 (2H, m), 7.52-7.62 (1H, m), 7.68-7.81 (2H, m), 7.84 (1H, dt, J=7.7, 1.5 Hz), 7.99 (1H, t, J=1.8 Hz).

### Working Example 286

### N-(2-Hydroxyethyl)-3-[2-[(6-methoxypyridin-3-yl)methyl]-1-benzothiophen-7-yl]benzamide

3-[2-[(6-Methoxypyridin-3-yl)methyl]-1-benzothiophen-7-yl]benzoic acid obtained in Reference Example 304, and 2-aminoethanol were used in the same manner as in Working Example 3 to obtain the titled compound. Yield: 56% melting point: 138-139° C. (hexane-ethyl acetate)

¹H-NMR (CDCl₃) δ: 2.69 (1H, t, J=4.8 Hz), 3.61-3.71 (2H, m), 3.86 (2H, q, J=4.9 Hz), 3.92 (3H, s), 4.14 (2H, s), 6.63 (1H, br s), 6.70 (1H, d, J=8.5 Hz), 7.10 (1H, s), 7.28-7.34 (1H, m), 7.38-7.45 (1H, m), 7.48 (1H, dd, J=8.5, 2.5 Hz), 7.51-7.59 (1H, m), 7.67 (1H, dd, J=7.7, 1.1 Hz), 7.77-7.87 (2H, m), 8.00-8.17 (2H, m).

### Working Example 287

### N-(2-Hydroxyethyl)-3-[2-[(2-methoxypyridin-3-yl)methyl]-1-benzothiophen-7-yl]benzamide

3-[2-[(2-Methoxypyridin-3-yl)methyl]-1-benzothiophen-7-yl]benzoic acid obtained in Reference Example 305, and 2-aminoethanol were used in the same manner as in Working Example 3 to obtain the titled compound. Yield: 59% melting point: 121-122° C. (hexane-ethyl acetate)

¹H-NMR (CDCl₃) δ: 3.59-3.72 (2H, m), 3.79-3.88 (2H, m), 3.97 (3H, s), 4.16 (2H, s), 6.63 (1H, br s), 6.82 (1H, dd, J=7.1, 5.2 Hz), 7.11 (1H, s), 7.30 (1H, d, J=7.4 Hz), 7.36-7.47 (2H, m), 7.50-7.59 (1H, m), 7.67 (1H, d, J=8.0 Hz), 7.83 (2H, d, J=7.7 Hz), 8.06 (2H, d, J=1.9 Hz). 1H unconfirmed.

### Working Example 288

### N-(2-Hydroxyethyl)-3-[2-[(6-oxo-1,6-dihydropyridin-3-yl)methyl]-1-benzothiophen-7-yl]benzamide

3-[2-[(6-Oxo-1,6-dihydropyridin-3-yl)methyl]-1-benzothiophen-7-yl]benzoic acid obtained in Reference Example 306, and 2-aminoethanol were used in the same manner as in Working Example 3 to obtain the titled compound. Yield: 17% melting point: 134-137° C. (methanol-ethyl acetate-)

¹H-NMR (DMSO-d₆) δ: 3.34.-3.41 (2H, m), 3.44-3.62 (2H, m), 3.97 (2H, s), 4.71 (1H, brs), 6.27 (1H, d, J=9.9 Hz), 7.24-7.42 (4H, m), 7.42-7.51 (1H, m), 7.54-7.64 (1H, m), 7.79 (2H, t, J=7.7 Hz), 7.89 (1H, d, J=7.7 Hz), 8.10 (1H, s), 8.53 (1H, t, J=5.6 Hz), 11.44 (1H, brs).

### Working Example 289

### N-(2-Hydroxyethyl)-3-[2-[(2-oxo-1,2-dihydropyridin-3-yl)methyl]-1-benzothiophen-7-yl]benzamide

3-[2-[(2-Oxo-1,2-dihydropyridin-3-yl)methyl]-1-benzothiophen-7-yl]benzoic acid obtained in Reference Example 307, and 2-aminoethanol were used in the same manner as in Working Example 3 to obtain the titled compound. Yield: 43%, amorphous solid.

¹H-NMR (DMSO-d₆) δ: 3.34-3.40 (2H, m), 3.51 (2H, q, J=6.0 Hz), 3.98 (2H, s), 4.71 (1H, t, J=5.6 Hz), 6.05-6.19 (1H, m), 7.19-7.32 (2H, m), 7.37 (2H, t, J=6.7 Hz), 7.41-7.48 (1H, m), 7.54-7.67 (1H, m), 7.72-7.85 (2H, m), 7.85-7.94 (1H, m), 8.10 (1H, d, J=1.4 Hz), 8.53 (1H, t, J=5.4 Hz), 11.61 (1H, br s).

### Working Example 290

### N-(2-Hydroxyethyl)-3-[2-[(1-methyl-6-oxo-1,6-dihydropyridin-3-yl)methyl]-1-benhen-7-yl]benzamide

3-[2-[(1-Methyl-6-oxo-1,6-dihydropyridin-3-yl)methyl]-1-benzothiophen-7-yl]benzoic acid obtained in Reference Example 310, and 2-aminoethanol were used in the same manner as in Working Example 3 to obtain the titled compound. Yield: 53%, amorphous solid.

¹H-NMR (CDCl₃) δ: 3.50 (3H, s), 3.60-3.72 (2H, m), 3.85 (2H, t, J=3.7 Hz), 3.94 (2H, s), 6.53 (1H, s), 7.14 (2H, d, J=11.0 Hz), 7.19-7.24 (1H, m), 7.32 (1H, d, J=7.1 Hz), 7.38-7.48 (1H, m), 7.54 (1H, s), 7.69 (1H, d, J=7.7 Hz), 7.81 (2H, t, J=8.1 Hz), 8.07 (1H, s).2H unconfirmed.

### Working Example 291

### N-(2-Hydroxyethyl)-3-[2-[(1-methyl-2-oxo-1,2-dihydropyridin-3-yl)methyl]-1-benzothiophen-7-yl]benzamide

3-[2-[(1-Methyl-2-oxo-1,2-dihydropyridin-3-yl)methyl]-1-benzothiophen-7-yl]benzoic acid obtained in Reference Example 312, and 2-aminoethanol were used in the same manner as in Working Example 3 to obtain the titled compound. Yield: 41% melting point: 173-174° C. (methanol-ethyl acetate-)

¹H-NMR (CDCl₃) δ: 3.10 (1H, t, J=5.1 Hz), 3.55 (3H, s), 3.60-3.70 (2H, m), 3.87 (2H, q, J=4.9 Hz), 4.12 (2H, s), 6.10 (1H, t, J=6.7 Hz), 6.80 (1H, br s), 7.15-7.22 (2H, m), 7.23-7.31 (2H, m), 7.35-7.43 (1H, m), 7.52 (1H, t, J=7.7 Hz), 7.67 (1H, dd, J=7.8, 1.2 Hz), 7.75-7.87 (2H, m), 8.09 (1H, t, J=1.5 Hz).

### Working Example 292

### N-(2-Hydroxyethyl)-3-[2-[[1-(1-methylethyl)-6-oxo-1,6-dihydropyridin-3-yl]methyl]-1-benzothiophen-7-yl]benzamide

1 N sodium hydroxide aqueous solution (1.11 mL, 1.11 mmol) was added to an ethanol (5 mL) solution of ethyl 3-[2-[[1-(1-methylethyl)-6-oxo-1,6-dihydropyridin-3-yl]methyl]-1-benzothiophen-7-yl]benzoate (0.16 g, 0.37 mmol) obtained in Reference Example 314, and the mixture was stirred for 30 min at 50° C. The reaction solution was neutralized with the addition of 1 N hydrochloric acid (1.11 mL), and the solvent was distilled off at reduced pressure. A mixture of the residue, 2-aminoethanol (0.027 mL, 0.44 mmol), WSC (0.096 g, 0.56 mmol), HOBt (0.075 g, 0.56 mmol), and DMF (5 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (hexane:ethyl acetate=1:1, then ethyl acetate:methanol=4:1) and was recrystallized from hexane-ethyl acetate to give 0.11 g of the titled compound (yield 66%).

Melting point: 178-183° C.

¹H NMR (DMSO-d₆) δ: 1.26 (6H, d, J=6.9 Hz), 3.30-3.40 (2H, m), 3.45-3.60 (2H, m), 4.02 (2H, s), 4.65-4.80 (1H, m), 4.95-5.10 (1H, m), 6.32 (1H, d, J=9.0 Hz), 7.20-7.35 (2H, m), 7.38 (1H, d, J=7.2 Hz), 7.45 (1H, d, J=7.8 Hz), 7.62 (1H, t, J=7.5 Hz), 7.70-7.85 (3H, m), 7.89 (1H, d, J=6.9 Hz), 8.12 (1H, s), 8.53 (1H, br s).

### Working Example 293

### N-(2-Hydroxyethyl)-3-[2-[[6-(1-methylethoxy)pyridin-3-yl]methyl]-1-benzothiophen-7-yl]benzamide

1 N sodium hydroxide aqueous solution (2.78 mL, 2.78 mmol) was added to an ethanol (5 mL) solution of ethyl 3-[2-[[6-(1-methylethoxy)-pyridin-3-yl]methyl]-1-benzothiophen-7-yl]benzoate (0.40 g, 0.93 mmol) obtained in Reference Example 315, and the mixture was stirred for 1 hour at 50° C. The reaction solution was neutralized with the addition of I N hydrochloric acid (2.78 mL), and the solvent was distilled off at reduced pressure. A mixture of the residue, 2-aminoethanol (0.074 mL, 1.11 mmol), WSC (0.24 g, 1.39 mmol), HOBt (0.19 g, 0.19 mmol), and DMF (5 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate) and recrystallized from hexane-ethyl acetate to give 0.34 g of the titled compound (yield 82%). Melting point: 106-107° C.

¹H NMR (CDCl₃) δ: 1.34 (6H, d, J=6.0 Hz), 2.68 (1H, t, J=5.4 Hz), 3.65 (2H, q, J=5.0 Hz), 3.86 (2H, q, J=5.0 Hz), 4.12 (2H, s), 5.20-5.30 (1H, m), 6.62 (1H, d, J=8.7 Hz), 6.60-6.70 (1H, m), 7.09 (1H, s), 7.30 (1H, d, J=7.2 Hz), 7.41 (1H, t, J=7.7 Hz), 7.40-7.50 (1H, m), 7.54 (1H, t, J=7.5 Hz), 7.67 (1H, d, J=8.1 Hz), 7.78-7.90 (2H, m), 8.05-8.10 (2H, m).

### Working Example 294

### N-(2-Hydroxyethyl)-3-[2-[[6-oxo-1-(2,2,2-trifluoroethyl)-1,6-dihydropyridin-3-yl]methyl]-1-benzothiophen-7-yl]benzamide

1 N sodium hydroxide aqueous solution (1.27 mL, 1.27 mmol) was added to an ethanol (5 mL) solution of ethyl 3-[2-[[6-oxo-1-(2,2,2-trifluoroethyl)-1,6-dihydropyridin-3-yl]methyl]-1-benzothiophen-7-yl]benzoate (0.20 g, 0.42 mmol) obtained in Reference Example 316, and the mixture was stirred for 30 min at 50° C. The reaction solution was neutralized with the addition of 1 N hydrochloric acid (1.27 mL), and the solvent was distilled off at reduced pressure. A mixture of the residue, 2-aminoethanol (0.03 mL, 0.51 mmol), WSC (0.11 g, 0.64 mmol), HOBt (0.086 g, 0.64 mmol), and DMF (5 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate:methanol=5:1) to give 0.068 g of the titled compound (yield 33%) in the form of amorphous solids.

¹H NMR (DMSO-d₆) δ: 3.30-3.45 (2H, m), 3.51 (2H, q, J=6.3 Hz), 4.03 (2H, s), 4.72 (1H, t, J=5.6 Hz), 4.81 (2H, q, J=9.5 Hz), 6.45 (1H, d, J=9.3 Hz), 7.32 (1H, s), 7.38-7.55 (3H, m), 7.60 (1H, t, J=7.5 Hz), 7.65 (1H, s), 7.75-7.85 (2H, m), 7.90 (1H, d, J=8.1 Hz), 8.12 (1H, s), 8.54 (1H, m).

### Working Example 295

### N-(2-Hydroxyethyl)-3-[2-[[6-(2,2,2-trifluoroethoxy)pyridin-3-yl]methyl]-1-benzothiophen-7-yl]benzamide

1 N sodium hydroxide aqueous solution (2.04 mL, 2.04 mmol) was added to an ethanol (5 mL) solution of ethyl 3-[2-[[6-(2,2,2-trifluoroethoxy)pyridin-3-yl]methyl]-1-benzothiophen-7-yl]benzoate (0.32 g, 0.68 mmol) obtained in Reference Example 317, and the mixture was stirred for 1 hour at 50° C. The reaction solution was neutralized with the addition of 1 N hydrochloric acid (2.04 mL), and the solvent was distilled off at reduced pressure. A mixture of the residue, 2-aminoethanol (0.054 mL, 0.81 mmol), WSC (0.18 g, 1.02 mmol), HOBt (0.14 g, 1.02 mmol), and DMF (5 mL) was stirred for 2.5 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate) and recrystallized from hexane-ethyl acetate to give 0.34 g of the titled compound (yield 82%). Melting point: 123-124° C.

1H NMR (CDCl₃) δ: 2.45 (1H, t, J=6.0 Hz), 3.66 (2H, q, J=5.1 Hz), 3.85 (2H, q, J=5.1 Hz), 4.15 (2H, s), 4.73 (2H, q, J=8.6 Hz), 6.55-6.70 (1H, m), 6.81 (1H, d, J=8.7 Hz), 7.09 (1H, s), 7.31 (1H, d, J=7.5 Hz), 7.42 (1H, t, J=8.4 Hz), 7.50-7.60 (2H, m), 7.67 (2H, d, J=8.1 Hz), 7.81 (2H, d, J=7.8 Hz), 8.06 (2H, s).

### Working Example 296

### N-(2-Hydroxyethyl)-3-[2-[[1-methyl-6-oxo-5-(trifluoromethyl)-1,6-dihydropyridin-3-yl]methyl]-1-benzothiophen-7-yl]benzamide

1 N sodium hydroxide aqueous solution (1.15 mL, 1.15 mmol) was added to an ethanol (5 mL) solution of ethyl 3-[2-[[1-methyl-6-oxo-5-(trifluoromethyl)-1,6-dihydropyridin-3-yl]methyl]-1-benzothiophen-7-yl]benzoate (0.18 g, 0.38 mmol) obtained in Reference Example 319, and the mixture was stirred for 1 hour at 50° C. The reaction solution was neutralized with the addition of 1 N hydrochloric acid (1.15 mL), and the solvent was distilled off at reduced pressure. A mixture of the residue, 2-aminoethanol (0.031 mL, 0.46 mmol), WSC (0.098 g, 0.57 mmol), HOBt (0.077 g, 0.57 mmol), and DMF (5 mL) was stirred for 1.5 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate, then ethyl acetate:methanol=10:1) to give 22.7 mg of the titled compound (yield 12%) in the form of amorphous solids.

¹H NMR (CDCl₃) δ: 2.45 (1H, br s), 3.56 (3H, s), 3.65 (2H, q, J=5.3 Hz), 3.85 (2H, t, J=4.2 Hz), 4.00 (2H, s), 6.64 (1H, br s), 7.15 (1H, s), 7.30-7.40 (2H, m), 7.45 (1H, t, J=7.5 Hz), 7.58 (1H, t, J=8.0 Hz), 7.71 (1H, d, J=7.2 Hz), 7.65 (1H, s), 7.75-7.85 (2H, m), 8.08 (1H, s).

### Working Example 297

### 3-[2-[(5-Chloro-1-methyl-6-oxo-1,6-dihydropyridin-3-yl)methyl]-1-benzothiophen-7-yl]-N-(2-hydroxyethyl)benzamide

1 N sodium hydroxide aqueous solution (3.56 mL, 3.56 mmol) was added to an ethanol (10 mL) solution of ethyl 3-[2-[(5-chloro-1-methyl-6-oxo-1,6-dihydropyridin-3-yl)methyl]-1-benzothiophen-7-yl]benzoate (0.52 g, 1.19 mmol) obtained in Reference Example 322, and the mixture was stirred for 30 min at 50° C. The reaction solution was neutralized with the addition of 1 N hydrochloric acid (3.56 mL), and the solvent was distilled off at reduced pressure. A mixture of the residue, 2-aminoethanol (0.085 mL, 1.42 mmol), WSC (0.34 g, 1.78 mmol), HOBt (0.24 g, 1.78 mmol), and DMF (5 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate:methanol=5:1) and recrystallized from hexane-ethyl acetate to give 0.34 g of the titled compound (yield 63%). Melting point: 210-211° C.

¹H NMR (CDCl₃) δ: 3.30-3.40 (2H, d, J=6.0 Hz), 3.48 (3H, s), 3.51 (2H, d, J=6.0 Hz), 4.00 (2H, s), 4.72 (1H, t, J=6.0 Hz), 7.34(1H, s), 7.40 (1H, d, J=7.5 Hz), 7.48 (1H, t, J=7.6 Hz), 7.60 (1H, t, J=7.8 Hz), 8.11 (1H, s), 8.50-8.60 (1H, m).

### Working Example 298

### N-(2-Methoxyethyl)-3-[2-[(1-methyl-2-oxo-1,2-dihydropyridin-4-yl)methyl]-1-benzothiophen-7-yl]benzamide

1 N sodium hydroxide aqueous solution (0.15 mL, 0.15 mmol) was added to an ethanol (1 mL) solution of ethyl 3-[2-[(1-methyl-2-oxo-1,2-dihydropyridin-4-yl)methyl]-1-benzothiophen-7-yl]benzoate (20.8 mg, 0.052 mmol) obtained in Reference Example 324, and the mixture was stirred for 1 hour at 60° C. The reaction solution was neutralized with the addition of 1 N hydrochloric acid (0.15 mL), and the solvent was distilled off at reduced pressure. A mixture of the residue, 2-methoxyethylamine (0.0054 mL, 0.062 mmol), WSC (13.3 mg, 0.077 mmol), HOBt (10.4 mg, 0.077 mmol), and DMF (1 mL) was stirred for 2 hours. The reaction solution was diluted with water and extracted with ethyl acetate. The extract was washed with water and dried over anhydrous magnesium sulfate, and the solvent was then distilled off at reduced pressure. The residue was purified by silica gel column chromatography (ethyl acetate:methanol=4:1) to give 6.8 mg of the titled compound (yield 30%) in the form of an oily substance.

¹H NMR (CDCl₃) δ: 3.38 (3H, s), 3.50 (3H, s), 3.58 (2H, q, J=5.4 Hz), 3.68 (2H, q, J=5.4 Hz), 4.00 (2H, s), 6.07 (1H, d, J=9.0 Hz), 6.49 (1H, s), 6.50-6.65 (1H, m), 7.15 (1H, s), 7.18 (1H, d, J=6.9 Hz), 7.33 (1H, d, J=7.2 Hz), 7.43 (1H, t, J=7.5 Hz), 7.54 (1H, t, J=7.5 Hz), 7.69 (1H, d, J=7.8 Hz), 7.77-7.85 (2H, m), 8.07 (1H, s).

### Working Example 299

### N-(2-Hydroxyethyl)-3-(1-methyl-2-[methyl[3-(trifluoromethyl)phenyl]amino]-1H-benzimidazol-4-yl)benzamide

3-(1-Methyl-2-[methyl[3-(trifluoromethyl)phenyl]amino]-1H-benzimidazol-4-yl)benzoic acid obtained in Reference Example 326, and 2-aminoethanol were used in the same manner as in Working Example 22 to obtain the titled compound. Yield: 90% melting point: 156-158° C. (ethyl acetate-hexane).

¹H-NMR (CDCl₃) δ: 2.87 (1H, t, J=5.1 Hz), 3.40 (3H, s), 3.56-3.65 (5H, m), 3.79 (2H, q, J=4.9 Hz), 6.90 (1H, br s), 6.99 (1H, dd, J=8.3, 2.3 Hz), 7.15 (1H, s), 7.22-7.30 (2H, m), 7.36 (2H, t, J=7.8 Hz), 7.49 (1H, d, J=6.4 Hz), 7.54 (1H, t, J=7.8 Hz), 7.78 (1H, d, J=8.3 Hz), 8.17 (1H, d, J=8.0 Hz), 8.48 (1H, s).

### Working Example 300

### N-(2-Oxopyrrolidin-3-yl)-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10, and 3-aminopyrrolidine-2-one dihydrochloride were used in the same manner as in Working Example 196 to obtain the titled compound. Yield: 56% melting point: 110-120° C. (ethyl acetate-hexane).

¹H-NMR (CDCl₃) δ: 1.97-2.18 (1H, m), 2.86-3.04 (1H, m), 3.45 (2H, dd, J=9.7, 4.0 Hz), 4.17 (2H, s), 4.45-4.61 (1H, m), 5.98 (1H, br s), 6.62 (1H, s), 6.87 (1H, d, J=4.5 Hz), 7.27-7.34 (2H, m), 7.39-7.46 (2H, m), 7.46-7.55 (3H, m), 7.57 (1H, s), 7.73 (1H, d, J=8.0 Hz), 7.79 (1H, d, J=7.6 Hz), 8.05 (1H, s).

### Working Example 301

### N-[2-(Methylamino)-2-oxoethyl]-3-[2-[3-(trifluoromethyl)benzyl]-1-benzofuran-4-yl]benzamide

3-[2-[3-(Trifluoromethyl)benzyl]-1-benzofuran-4-yl]benzoic acid obtained in Reference Example 10, and N-methylglycinamide hydrochloride were used in the same manner as in Working Example 196 to obtain the titled compound. Yield: 76% melting point: 135-139° C. (ethyl acetate-hexane).

¹H-NMR (CDCl₃) δ: 2.87 (3H, d, J=4.9 Hz), 4.14 (2H, d, J=4.9 Hz), 4.18 (2H, s), 6.11 (1H, br s), 6.61 (1H, s), 7.07 (1H, t, J=4.7 Hz), 7.30 (1H, s), 7.31 (1H, d, J=3.4 Hz), 7.39-7.46 (2H, m), 7.46-7.59 (4H, m), 7.75 (1H, d, J=8.0 Hz), 7.80 (1H, d, J=7.6 Hz), 8.06 (1H, s).

### Working Example 302

### Methyl N-[(3-[2-[3-(trifluoromethyl)benzyl]-1-benzofuran-4-yl]phenyl)carbonyl]glycinate

3-[2-[3-(Trifluoromethyl)benzyl]-1-benzofuran-4-yl]benzoic acid obtained in Reference Example 10, and methylglycinate hydrochloride were used in the same manner as in Working Example 196 to obtain the titled compound. Yield: 98%. Oily Substance.

¹H-NMR (CDCl₃) δ: 3.81 (3H, s), 4.18 (2H, s), 4.28 (2H, d, J=5.1 Hz), 6.62 (1H, d, J=0.8 Hz), 6.70 (1H, br s), 7.28-7.36 (2H, m), 7.40-7.59 (6H, m), 7.72-7.78 (1H, m), 7.78-7.83 (1H, m), 8.06 (1H, t, J=1.7 Hz).

### Working Example 303

### N-[(3-[2-[3-(Trifluoromethyl)benzyl]-1-benzofuran-4-yl]phenyl)carbonyl]glycine

Methyl N-[(3-[2-[3-(trifluoromethyl)benzyl]-1-benzofuran-4-yl]phenyl)carbonyl]glycinate obtained in Working Example 302 was used in the same manner as in Reference Example 5 to obtain the titled compound. Yield: 74%, amorphous solid.

¹H-NMR (CDCl₃) δ: 4.17 (2H, s), 4.31 (2H, d, J=5.1 Hz), 6.61 (1H, d, J=0.8 Hz), 6.79 (1H, s), 7.28-7.35 (2H, m), 7.39-7.46 (2H, m), 7.46-7.59 (4H, m), 7.71-7.82 (2H, m), 8.04 (1H, t, J=1.6 Hz), 1H unconfirmed.

### Working Example 304

### N-[2-(Dimethylamino)-2-oxoethyl]-3-[2-[3-(trifluoromethyl)benzyl]-1-benzofuran-4-yl]benzamide

N-[(3-[2-[3-(Trifluoromethyl)benzyl]-1-benzofuran-4-yl]phenyl)carbonyl]glycine obtained in Working Example 303, and dimethylamine hydrochloride were used in the same manner as in Working Example 196 to obtain the titled compound. Yield: 49%, amorphous solid.

¹H-NMR (CDCl₃) δ: 3.05 (6H, d, J=3.4 Hz), 4.18 (2H, s), 4.27 (2H, d, J=3.8 Hz), 6.63 (1H, d, J=0.8 Hz), 7.29-7.34 (2H, m), 7.38-7.46 (3H, m), 7.47-7.60 (4H, m), 7.71-7.77 (1H, m), 7.80-7.86 (1H, m), 8.09 (1H, t, J=1.7 Hz).

### Working Example 305

### N-[2-[(2-Hydroxyethyl)amino]-2-oxoethyl]-3-[2-[3-(trifluoromethyl)benzyl]-1-benzofuran-4-yl]benzamide

N-[(3-[2-[3-(Trifluoromethyl)benzyl]-1-benzofuran-4-yl]phenyl)carbonyl]glycine obtained in Working Example 303, and 2-aminoethanol were used in the same manner as in Working Example 22 to obtain the titled compound. Yield: 55% melting point: 89-94° C. (ethyl acetate-hexane).

¹H-NMR (CDCl₃ ) δ: 2.76 (1H, t, J=5.4 Hz), 3.47 (2H, q, J=5.4 Hz), 3.74 (2H, q, J=5.0 Hz), 4.12-4.27 (4H, m), 6.61 (1H, s), 6.79 (1H, br s), 7.23 (1H, br s), 7.27-7.34 (2H, m), 7.38-7.46 (2H, m), 7.46-7.59 (4H, m), 7.74 (1H, d, J=7.9 Hz), 7.80 (1H, d, J=7.7 Hz), 8.05 (1H, s).

### Working Example 306

### N-(2-Hydroxyethyl)-2-[2-[3-(trifluoromethyl)benzyl]-1-benzothiophen-7-yl]pyridine-4-carboxamide

N-[(3-[2-[3-(Trifluoromethyl)benzyl]-1-benzofuran-4-yl]phenyl)carbonyl]glycine obtained in Working Example 303, and pyrrolizidine were used in the same manner as in Working Example 3 to obtain the titled compound. Yield: 81%, amorphous solid.

¹H-NMR (CDCl₃) δ: 1.84-1.98 (2H, m), 1.98-2.10 (2H, m), 3.46 (2H, t, J=6.7 Hz), 3.55 (2H, t, J=6.9 Hz), 4.18 (2H, s), 4.20 (2H, d, J=4.0 Hz), 6.63 (1H, d, J=0.8 Hz), 7.30-7.34 (2H, m), 7.36 (1H, br s), 7.40-7.46 (2H, m), 7.48-7.53 (2H, m), 7.54-7.60 (2H, m), 7.71-7.77 (1H, m), 7.80-7.86 (1H, m), 8.09 (1H, t, J=1.6 Hz).

### Working Example 307

### N-(2-Hydroxyethyl)-3-(2-[[6-(trifluoromethyl)pyridin-2-yl]methyl]-1-benzothiophen-4-benzamide

3-(2-[[6-(Trifluoromethyl)pyridin-2-yl]methyl]-1-benzothiophen-4-yl)benzoic acid obtained in Reference Example 331, and 2-aminoethanol were used in the same manner as in Working Example 22 to obtain the titled compound. Yield: 50% melting point: 123-124° C. (ethyl acetate-hexane).

¹H-NMR (CDCl₃) δ: 2.54 (1H, t, J=4.9 Hz), 3.66 (2H, q, J=5.3 Hz), 3.86 (2H, q, J=4.8 Hz), 4.47 (2H, s), 6.69 (1H, br s), 7.24 (1H, s), 7.28-7.39 (2H, m), 7.41 (1H, d, J=7.9 Hz), 7.51-7.59 (2H, m), 7.69 (1H, d, J=7.7 Hz), 7.74-7.85 (3H, m), 7.95 (1H, s).

### Working Example 308

### N-(2-Hydroxyethyl)-3-(2-[[6-(trifluoromethyl)pyridin-2-yl]methyl]-1-benzothiophen-7-yl) benzamide

3-(2-[[6-(Trifluoromethyl)pyridin-2-yl]methyl]-1-benzothiophen-7-yl)benzoic acid obtained in Reference Example 330, and 2-aminoethanol were used in the same manner as in Working Example 22 to obtain the titled compound. Yield: 73%, amorphous solid.

¹H-NMR (CDCl₃) δ: 2.47 (1H, t, J=5.1 Hz), 3.60-3.72 (2H, m), 3.85 (2H, q, J=5.2 Hz), 4.48 (2H, s), 6.64 (1H, br s), 7.23 (1H, s), 7.33 (1H, d, J=6.4 Hz), 7.39-7.47 (2H, m), 7.55 (2H, dt, J=7.4, 3.9 Hz), 7.71 (1H, d, J=6.8 Hz), 7.74-7.87 (3H, m), 8.07 (1H, s).

### Working Example 309

### N-(2-Hydroxyethyl)-3-(2-[[3-(trifluoromethyl)benzyl]oxy]pyrazolo[1,5-a]pyridin-7-yl)benzamide

3-(2-[[3-(Trifluoromethyl)benzyl]oxy]pyrazolo[1,5-a]pyridin-7-yl)benzoic acid obtained in Reference Example 334, and 2-aminoethanol were used in the same manner as in Working Example 22 to obtain the titled compound. Yield: 58% melting point: 118-119° C. (ethyl acetate-hexane).

¹H-NMR (CDCl₃) δ: 2.49 (1H, t, J=5.1 Hz), 3.58-3.70 (2H, m), 3.84 (2H, q, J=5.1 Hz), 5.32 (2H, s), 5.98 (1H, s), 6.73 (2H, dd, J=7.2, 1.5 Hz), 7.15 (1H, dd, J=8.7, 7.2 Hz), 7.34 (1H, dd, J=8.9, 1.3 Hz), 7.44-7.51 (1H, m), 7.52-7.59 (2H, m), 7.64 (1H, d, J=7.9 Hz), 7.73 (1H, s), 7.88 (1H, d, J=8.3 Hz), 8.02 (1H, d, J=7.9 Hz), 8.29 (1H, s).

### Working Example 310

### 3-[2-(3-Fluorobenzyl)-1-benzothiophen-7-yl]-N-(2-methoxyethyl)benzamide

3-[2-(Bromomethyl)-1-benzothiophene-7-yl]- obtained in Reference Example 187N-(2-methoxyethyl)benzamide, and (3-fluorophenyl)boronic acid were used in the same manner as in Working Example 9 to obtain the titled compound. Yield: 57% melting point: 108-110° C. (ethyl acetate-hexane).

¹H-NMR (CDCl₃) δ: 3.37 (3H, s), 3.53-3.60 (2H, m), 3.63-3.72 (2H, m), 4.21 (2H, s), 6.54 (1H, brs), 6.88-7.01 (2H, m), 7.06 (1H, d, J=7.5 Hz), 7.11 (1H, s), 7.21-7.29 (1H, m), 7.32 (1H, d, J=7.5 Hz), 7.38-7.47 (1H, m), 7.49-7.59 (1H, m), 7.69 (1H, d, J=7.9 Hz), 7.77-7.86 (2H, m), 8.05 (1H, s).

### Working Example 311

### 3-[2-(3-Chlorobenzyl)-1-benzothiophen-7-yl]-N-(2-methoxyethyl)benzamide

3-[2-(Bromomethyl)-1-benzothiophene-7-yl]- obtained in Reference Example 187N-(2-methoxyethyl)benzamide, and (3-chlorophenyl)boronic acid were used in the same manner as in Working Example 9 to obtain the titled compound. Yield: 61% melting point: 120-134° C. (ethyl acetate-hexane).

¹H-NMR (CDCl₃) δ: 3.37 (3H, s), 3.53-3.61 (2H, m), 3.68 (2H, q, J=4.9 Hz), 4.19 (2H, s), 6.54 (1H, brs), 7.11 (1H, s), 7.13-7.19 (1H, m), 7.19-7.28 (3H, m), 7.32 (1H, d, J=6.4 Hz), 7.39-7.47 (1H, m), 7.50-7.58 (1H, m), 7.69 (1H, d, J=6.8 Hz), 7.82 (2H, td, J=5.1, 2.3 Hz), 8.05 (1H, s).

### Working Example 312

### 3-Fluoro-5-[3-methyl-2-[3-(trifluoromethyl)benzyl]-1-benzothiophen-7-yl]benzamide

3-Fluoro-5-[3-methyl-2-[3-(trifluoromethyl)benzyl]-1-benzothiophen-7-yl]benzoic acid obtained in Reference Example 338 was used in the same manner as in Working Example 197 to obtain the titled compound. Yield: 70% melting point: 146-147° C. (ethyl acetate-hexane).

¹H-NMR (CDCl₃) δ: 2.42 (3H, s), 4.27 (2H, s), 5.63 (1H, br s), 6.01 (1H, br s), 7.35 (1H, dd, J=7.6, 1.1 Hz), 7.40 (2H, dd, J=4.7, 1.7 Hz), 7.48 (2H, d, J=3.0 Hz), 7.49-7.53 (1H, m), 7.53-7.56 (1H, m), 7.57 (1H, t, J=1.7 Hz), 7.70 (1H, dd, J=8.0, 1.1 Hz), 7.86 (1H, t, J=1.5 Hz).

### Working Example 313

### 3-Fluoro-N-(2-hydroxyethyl)-5-[3-methyl-2-[3-(trifluoromethyl)benzyl]-1-benzothiophen-7-yl]benzamide

3-Fluoro-5-[3-methyl-2-[3-(trifluoromethyl)benzyl]-1-benzothiophen-7-yl]benzoic acid obtained in Reference Example 338, and 2-aminoethanol were used in the same manner as in Working Example 22 to obtain the titled compound. Yield: 78% melting point: 151-152° C. (ethyl acetate-hexane).

¹H-NMR (CDCl₃) δ: 2.23 (1H, t, J=5.1 Hz), 2.42 (3H, s), 3.61-3.70 (2H, m), 3.84 (2H, q, J=4.9 Hz), 4.27 (2H, s), 6.57 (1H, br s), 7.34 (1H, d, J=7.2 Hz), 7.39 (1H, d, J=1.9 Hz), 7.40 (1H, s), 7.43-7.53 (4H, m), 7.55 (1H, s), 7.70 (1H, d, J=6.8 Hz), 7.82 (1H, s).

### Working Example 314

### 3-Fluoro-N-(2-methoxyethyl)-5-[3-methyl-2-[3-(trifluoromethyl)benzyl]-1-benzothiophen-7-yl]benzamide

3-Fluoro-5-[3-methyl-2-[3-(trifluoromethyl)benzyl]-1-benzothiophen-7-yl]benzoic acid obtained in Reference Example 338, and 2-methoxyethanamine were used in the same manner as in Working Example 3 to obtain the titled compound. Yield: 83% melting point: 124-126° C. (ethyl acetate-hexane).

¹H-NMR (CDCl₃) δ: 2.42 (3H, s), 3.36 (3H, s), 3.52-3.59 (2H, m), 3.66 (2H, q, J=4.8 Hz), 4.27 (2H, s), 6.49 (1H, br s), 7.35 (1H, d, J=7.6 Hz), 7.39 (1H, d, J=1.9 Hz), 7.40 (1H, br s), 7.43-7.53 (4H, m), 7.54 (1H, s), 7.70 (1H, d, J=7.2 Hz), 7.81 (1H, s).

### Working Example 315

### N-(2-Amino-2-oxoethyl)-3-fluoro-5-[3-methyl-2-[3-(trifluoromethyl)benzyl]-1-benzothiophen-7-yl]benzamide

3-Fluoro-5-[3-methyl-2-[3-(trifluoromethyl)benzyl]-1-benzothiophen-7-yl]benzoic acid obtained in Reference Example 338, and glycinamide hydrochloride were used in the same manner as in Working Example 196 to obtain the titled compound. Yield: 69% melting point: 46-47° C. (ethyl acetate-hexane).

¹H-NMR (CDCl₃) δ: 2.41 (3H, s), 4.17 (2H, d, J=4.9 Hz), 4.27 (2H, s), 5.47 (1H, br s), 5.91 (1H, br s), 6.95 (1H, br s), 7.34 (1H, d, J=6.4 Hz), 7.37-7.42 (2H, m), 7.43-7.52 (3H, m), 7.54 (1H, s), 7.57 (1H, s), 7.69 (1H, d, J=7.2 Hz), 7.87 (1H, s).

### Working Example 316

### 3-(2-[Hydroxy[3-(trifluoromethyl)phenyl]methyl]-1-benzothiophen-7-yl)benzamide

3-(2-[Hydroxy[3-(trifluoromethyl)phenyl] methyl]-1-benzothiophen-7-yl)benzoic acid obtained in Reference Example 341 was used in the same manner as in Working Example 197 to obtain the titled compound. Yield: 55%, amorphous solid.

¹H-NMR (CDCl₃) δ: 2.72 (1H, d, J=3.8 Hz), 5.63 (1H, br s), 6.10 (1H, br s), 6.18 (1H, d, J=3.4 Hz), 7.24 (1H, s), 7.32-7.39 (1H, m), 7.41-7.48 (1H, m), 7.48-7.55 (1H, m), 7.55-7.61 (2H, m), 7.67 (1H, d, J=7.6 Hz), 7.72 (1H, d, J=8.0 Hz), 7.77 (1H, s), 7.85 (2H, d, J=7.6 Hz), 8.08 (1H, s).

### Working Example 317

### N-(2-Hydroxyethyl)-3-(2-[hydroxy[3-(trifluoromethyl)phenyl]methyl]-1-benzothiophen-7-yl)benzamide

3-(2-[Hydroxy[3-(trifluoromethyl)phenyl]methyl]-1-benzothiophen-7-yl)benzoic acid obtained in Reference Example 341, and 2-aminoethanol were used in the same manner as in Working Example 22 to obtain the titled compound. Yield: 19%, amorphous solid.

¹H-NMR (CDCl₃) δ: 2.47 (1H, t, J=5.1 Hz), 2.79 (1H, d, J=3.8 Hz), 3.59-3.71 (2H, m), 3.84 (2H, q, J=4.9 Hz), 6.18 (1H, d, J=3.8 Hz), 6.65 (1H, brs), 7.23 (1H, s), 7.31-7.37 (1H, m), 7.41-7.47 (1H, m), 7.48-7.62 (3H, m), 7.66 (1H, d, J=7.6 Hz), 7.71 (1H, d, J=8.0 Hz), 7.77 (1H, s), 7.78-7.85 (2H, m), 8.04 (1H, s).

### Working Example 318

### 3-(2-[Hydroxy[3-(trifluoromethyl)phenyl]methyl]-1-benzothiophen-7-yl)-N-(2-methoxyethyl)benzamide

3-(2-[Hydroxy[3-(trifluoromethyl)phenyl]methyl]-1-benzothiophen-7-yl)benzoic acid obtained in Reference Example 341, and 2-methoxyethanamine were used in the same manner as in Working Example 3 to obtain the titled compound. Yield: 78% melting point: 126-127° C. (ethyl acetate-hexane).

¹H-NMR (CDCl₃) δ: 2.73 (1H, d, J=3.8 Hz), 3.37 (3H, s), 3.52-3.61 (2H, m), 3.62-3.71 (2H, m), 6.18 (1H, d, J=3.4 Hz), 6.55 (1H, br s), 7.24 (1H, s), 7.31-7.38 (1H, m), 7.41-7.46 (1H, m), 7.47-7.60 (3H, m), 7.66 (1H, d, J=8.0 Hz), 7.72 (1H, d, J=7.6 Hz), 7.77 (1H, s), 7.78-7.86 (2H, m), 8.03 (1H, s).

### Working Example 319

### N-(2-Amino-2-oxoethyl)-3-(2-[hydroxy[3-(trifluoromethyl)phenyl]methyl]-1-benzothiophen-7-yl)benzamide

3-(2-[Hydroxy[3-(trifluoromethyl)phenyl]methyl]-1-benzothiophen-7-yl)benzoic acid obtained in Reference Example 341, and glycinamide hydrochloride were used in the same manner as in Working Example 196 to obtain the titled compound. Yield: 60%, amorphous solid.

¹H-NMR (CDCl₃) δ: 3.65 (1H, d, J=3.8 Hz), 4.13 (2H, d, J=4.9 Hz), 5.72 (1H, br s), 6.13 (1H, d, J=3.4 Hz), 6.40 (1H, br s), 7.14 (1H, s), 7.21-7.34 (3H, m), 7.35-7.51 (3H, m), 7.51-7.58 (1H, m), 7.60-7.69 (2H, m), 7.69-7.81 (2H, m), 8.08 (1H, s).

### Working Example 320

### 3-(2-[1-Hydroxy-1-[3-(trifluoromethyl)phenyl]ethyl]-1-benzothiophen-7-yl)benzamide

3-(2-[1-Hydroxy-1-[3-(trifluoromethyl)phenyl]ethyl]-1-benzothiophen-7-yl)benzoic acid obtained in Reference Example 346 was used in the same manner as in Working Example 197 to obtain the titled compound. Yield: 62%, amorphous solid.

¹H-NMR (CDCl₃ ) δ: 2.09 (3H, s), 2.77 (1H, s), 5.69 (1H, br s), 6.13 (1H, br s), 7.24 (1H, s), 7.31-7.37 (1H, m), 7.44 (2H, t, J=7.5 Hz), 7.50-7.59 (2H, m), 7.68 (1H, d, J=7.9 Hz), 7.72 (1H, dd, J=7.9, 1.1 Hz), 7.79-7.90 (3H, m), 8.06 (1H, s).

### Working Example 321

### N-(2-Hydroxyethyl)-3-(2-[1-hydroxy-1-[3-(trifluoromethyl)phenyl]ethyl]-1-benzothiophen-7-yl)benzamide

3-(2-[1-Hydroxy-1-[3-(trifluoromethyl)phenyl]ethyl]-1-benzothiophen-7-yl)benzoic acid obtained in Reference Example 346, and 2-aminoethanol were used in the same manner as in Working Example 3 to obtain the titled compound. Yield: 65% melting point: 176-177° C. (ethyl acetate-hexane).

¹H-NMR (CDCl₃) δ: 2.09 (3H, s), 2.45 (1H, t, J=4.9 Hz), 2.71 (1H, s), 3.64 (2H, q, J=5.3 Hz), 3.84 (2H, q, J=4.8 Hz), 6.64 (1H, br s), 7.24 (1H, s), 7.33 (1H, d, J=6.0 Hz), 7.44 (2H, t, J=7.5 Hz), 7.49-7.57 (2H, m), 7.68 (1H, d, J=7.9 Hz), 7.72 (1H, d, J=6.8 Hz), 7.80 (2H, t, J=7.9 Hz), 7.86 (1H, s), 8.02 (1H, s).

### Working Example 322

### 3-(2-[1-Hydroxy-1-[3-(trifluoromethyl)phenyl]ethyl]-1-benzothiophen-7-yl)-N-(2-methoxy ethyl)benzamide

3-(2-[1-Hydroxy-1-[3-(trifluoromethyl)phenyl]ethyl]-1-benzothiophen-7-yl)benzoic acid obtained in Reference Example 346, and 2-methoxyethanamine were used in the same manner as in Working Example 3 to obtain the titled compound. Yield: 80% melting point: 134-138° C. (ethyl acetate-hexane).

¹H-NMR (CDCl₃) δ: 2.09 (3H, s), 2.60 (1H, s), 3.37 (3H, s), 3.52-3.60 (2H, m), 3.67 (2H, q, J=4.8 Hz), 6.54 (1H, br s), 7.25 (1H, s), 7.32-7.39 (1H, m), 7.41-7.49 (2H, m), 7.50-7.57 (2H, m), 7.68 (1H, d, J=7.9 Hz), 7.72 (1H, d, J=6.8 Hz), 7.77-7.84 (2H, m), 7.86 (1H, s), 8.02 (1H, s).

### Working Example 323

### N-(2-Amino-2-oxoethyl)-3-(2-[1-hydroxy-1-[3-(trifluoromethyl)phenyl]ethyl]-1-benzothio phen-7-yl)benzamide

3-(2-[1-Hydroxy-1-[3-(trifluoromethyl)phenyl]ethyl]-1-benzothiophen-7-yl)benzoic acid obtained in Reference Example 346, and glycinamide hydrochloride were used in the same manner as in Working Example 196 to obtain the titled compound. Yield: 47%, amorphous solid.

¹H-NMR (CDCl₃) δ: 2.03 (3H, s), 3.95 (1H, s), 4.06 (2H, d, J=4.9 Hz), 5.89 (1H, br s), 6.63 (1H, br s), 7.15 (1H, s), 7.20-7.29 (1H, m), 7.29-7.45 (4H, m), 7.45-7.53 (1H, m), 7.63 (2H, dd, J=7.3, 3.6 Hz), 7.71 (2H, d, J=7.5 Hz), 7.83 (1H, s), 8.04 (1H, s).

### Working Example 324

### N-(2-Hydroxyethyl)-3-(2-[1-[3-(trifluoromethyl)phenyl]ethyl]-1-benzothiophen-7-yl)benzamide

3-(2-[1-[3-(Trifluoromethyl)phenyl]ethyl]-1-benzothiophen-7-yl)benzoic acid obtained in Reference Example 349, and 2-aminoethanol were used in the same manner as in Working Example 3 to obtain the titled compound. Yield: 67%, amorphous solid.

¹H-NMR (CDCl₃) δ: 1.78 (3H, d, J=6.8 Hz), 2.42 (1H, br s), 3.65 (2H, d, J=4.9 Hz), 3.84 (2H, brs), 4.45 (1H, q, J=6.9 Hz), 6.61 (1H, brs), 7.13 (1H, s), 7.31 (1H, d, J=7.2 Hz), 7.37-7.60 (6H, m), 7.70 (1H, d, J=8.0 Hz), 7.81 (2H, br s), 8.04 (1H, s).

### Working Example 325

### N-(2-Amino-2-oxoethyl)-3-(2-[1-[3-(trifluoromethyl)phenyl]ethyl]-1-benzothiophen-7-yl)benzamide

3-(2-[1-[3-(Trifluoromethyl)phenyl]ethyl]-1-benzothiophen-7-yl)benzoic acid obtained in Reference Example 349, and glycinamide hydrochloride were used in the same manner as in Working Example 196 to obtain the titled compound. Yield: 44%, amorphous solid.

¹H-NMR (CDCl₃) δ: 1.77 (3H, d, J=6.8 Hz), 4.19 (2H, br s), 4.44 (1H, d, J=7.2 Hz), 5.53 (1H, br s), 6.23 (1H, br s), 7.06 (1H, br s), 7.12 (1H, s), 7.30 (1H, d, J=5.7 Hz), 7.37-7.59 (6H, m), 7.69 (1H, d, J=7.2 Hz), 7.82 (2H, br s), 8.09 (1H, br s).

### Working Example 326

### 4-[2-[3-(Trifluoromethyl)benzyl]-1-benzothiophen-7-yl]-2,3-dihydro-1H-isoindole-1-one

4,4,5,5-Tetramethyl-2-[2-[3-(trifluoromethyl)benzyl]-1-benzothiophene-7-yl]-1,3,2-dioxaborolane obtained in Reference Example 193, and 4-bromo-2,3-dihydro-1H-isoindole-1-one were used in the same manner as in Working Example 9 to obtain the titled compound. Yield: 54% melting point: 195-196° C. (ethyl acetate-hexane).

¹H-NMR (CDCl₃) δ: 4.25 (2H, s), 4.36 (2H, s), 6.40 (1H, br s), 7.12 (1H, s), 7.20-7.31 (1H, m), 7.37-7.48 (3H, m), 7.48-7.55 (2H, m), 7.55-7.64 (1H, m), 7.73 (2H, d, J=8.0 Hz), 7.94 (1H, d, J=6.8 Hz).

### Working Example 327

### 6-[2-[3-(Trifluoromethyl)benzyl]-1-benzothiophen-7-yl]-2,3-dihydro-1H-isoindole-1-one

4,4,5,5-Tetramethyl-2-[2-[3-(trifluoromethyl)benzyl]-1-benzothiophene-7-yl]-1,3,2-dioxaborolane obtained in Reference Example 193, and 6-bromo-2,3-dihydro-1H-isoindole-1-one were used in the same manner as in Working Example 9 to obtain the titled compound. Yield: 69% melting point: 151-152° C. (ethyl acetate-hexane).

¹H-NMR (CDCl₃) δ: 4.27 (2H, s), 4.52 (2H, s), 6.50 (1H, br s), 7.12 (1H, s), 7.34 (2H, d, J=6.4 Hz), 7.38-7.47 (3H, m), 7.48-7.52 (1H, m), 7.54 (1H, s), 7.57 (1H, d, J=8.3 Hz), 7.70 (1H, dd, J=8.0, 1.1 Hz), 7.92 (1H, dd, J=8.0, 1.5 Hz), 8.17 (1H, s).

### Working Example 328

### N-(2-Amino-2-oxoethyl)-3-[5-fluoro-2-[3-(trifluoromethyl)benzyl]-1-benzothiophen-7-yl]benzamide

A THF mixture (4 mL) of 3-[5-fluoro-2-[3-(trifluoromethyl)benzyl]-1-benzothiophen-7-yl]benzoic acid (200 mg, 0.47 mmol) obtained in Reference Example 354, glycinamide hydrochloride (67 mg, 0.61 mmol), N-ethyl diisopropylamine (0.11 mL, 0.61 mmol), N-[3-(dimethylamino)propyl]-N′-ethylcarbodiimide (116 mg, 0.61 mmol), and HOBt (82 mg, 0.61 mmol) was stirred for 3 hours at room temperature. DMF (1 mL) was added to the reaction solution, and the mixture was stirred for 3 hours at 50° C. The reaction solution was diluted with ethyl acetate, then washed with 1 N hydrochloric acid, sodium bicarbonate aqueous solution, and saturated brine, dried over anhydrous sodium sulfate, and then concentrated at reduced pressure. The residue was purified by silica gel column chromatography (ethyl acetate-methanol=90:10) and then crystallized in hexane to give 150 mg of the titled compound (yield 66%) in the form colorless crystals. Melting point: 71-73° C.

¹H-NMR (CDCl₃) δ: 4.20 (2H, d, J=4.9 Hz), 4.25 (2H, s), 5.56 (1H, br s), 6.20 (1H, br s), 7.02-7.16 (3H, m), 7.35 (1H, dd, J=9.0, 2.6 Hz), 7.39-7.59 (5H, m), 7.78-7.88 (2H, m), 8.10 (1H, t, J=1.7 Hz).

### Working Example 329

### N-(2-Amino-2-oxoethyl)-2-[2-[3-(trifluoromethyl)benzyl]-1-benzothiophen-4-yl]pyridine-4-carboxamide

2-[2-[3-(Trifluoromethyl)benzyl]-1-benzothiophen-4-yl]pyridine-4-carboxylic acid obtained in Reference Example 359, and glycinamide hydrochloride were used in the same manner as in Working Example 196 to obtain the titled compound in solid form. Yield. 69%, melting point: 49-50° C. (ethyl acetate-hexane)

¹H-NMR (DMSO-d₆) δ: 3.87 (2H, d, J=6.0 Hz), 4.43 (2H, s), 7.10 (1H, br s), 7.39-7.50 (2H, m), 7.51-7.67 (3H, m), 7.68-7.77 (2H, m), 7.77-7.85 (2H, m), 7.99 (1H, d, J=7.9 Hz), 8.21 (1H, s), 8.88 (1H, d, J=4.5 Hz), 9.12 (1H, t, J=5.8 Hz).

### Working Example 330

### N-(2-Amino-2-oxoethyl)-5-[2-[3-(trifluoromethyl)benzyl]-1-benzothiophen-7-yl]pyridine-3-carboxamide

5-[2-[3-(Trifluoromethyl)benzyl]-1-benzothiophen-7-yl]pyridine-3-carboxylic acid obtained in Reference Example 361, and glycinamide hydrochloride were used in the same manner as in Working Example 196 to obtain the titled compound in solid form. Yield. 19%, melting point: 122-123° C. (ethyl acetate-hexane)

¹H-NMR (DMSO-d₆) δ: 3.85 (2H, d, J=5.7 Hz), 4.40 (2H, s), 7.08 (1H, br s), 7.34-7.69 (7H, m), 7.72 (1H, s), 7.88 (1H, d, J=7.5 Hz), 8.49 (1H, s), 8.94-9.16 (3H, m).

### Working Example 331

### N-(2-Amino-2-oxoethyl)-4-[2-(3-chloro-5-fluorobenzyl)-1-benzothiophen-7-yl]pyridine-2-carboxamide

4-[2-(3-Chloro-5-fluorobenzyl)-1-benzothiophen-7-yl]pyridine-2-carboxylic acid obtained in Reference Example 362, and glycinamide hydrochloride were used in the same manner as in Working Example 196 to obtain the titled compound in solid form. Yield. 47%, melting point: 157-158° C. (ethyl acetate-hexane)

¹H-NMR (DMSO-d₆) δ: 3.93 (2H, d, J=5.7 Hz), 4.33 (2H, s), 7.14 (1H, br s), 7.19-7.27 (1H, m), 7.27-7.34 (2H, m), 7.42 (1H, s), 7.48 (1H, br s), 7.52-7.59 (2H, m), 7.88-7.96 (2H, m), 8.33-8.37 (1H, m), 8.81 (1H, d, J=4.9 Hz), 8.92 (1H, t, J=5.7 Hz).

### Working Example 332

### 3-Fluoro-N-(2-hydroxyethyl)-2-[2-[3-(trifluoromethyl)benzyl]-1-benzothiophen-7-yl]pyridine-4-carboxamide

A mixture of 2-chloro-3-fluoro-N-(2-hydroxyethyl)pyridine-4-carboxamide (188 mg, 0.861 mmol) obtained in Reference Example 363, 4,4,5,5-tetramethyl-2-[2-[3-(trifluoromethyl)benzyl]-1-benzothiophen-7-yl]-1,3,2-dioxaborolane (300 mg, 0.717 mmol) obtained in Reference Example 193, and tetrakis(triphenylphosphine)palladium (0) (99.5 mg, 0.086 mmol) in 2 N sodium carbonate aqueous solution (1.1 mL)-1,2-dimethoxyethane (6.0 mL) was stirred for 3 hours at 90° C. in a nitrogen atmosphere. Water was poured into the reaction solution, and the mixture was extracted with ethyl acetate. The extract was dried over anhydrous magnesium sulfate, and the solvent was distilled off at reduced pressure. The residue was purified by silica gel column chromatography (ethyl acetate:hexane=1.1), purified by HPLC (YMC HPLC column, eluateA: 0.1% trifluoroacetic acid acetonitrile solution; eluate B: 0.01% trifluoroacetic acid aqueous solution; eluted with 10% eluate A to 100% eluate A), and recrystallized from hexane and ethyl acetate to give 141 mg of the titled compound (yield 41%) in solid form. Melting point: 110-111° C.

¹H-NMR (DMSO-d₆) δ: 3.31-3.40 (2H, m), 3.53 (2H, q, J=5.8 Hz), 4.39 (2H, s), 4.77 (1H, t, J=5.8 Hz), 7.33 (1H, s), 7.47-7.68 (5H, m), 7.72 (1H, s), 7.84-7.96 (2H, m), 8.61-8.74 (2H, m).

### Working Example 333

### N-(2-Amino-2-oxoethyl)-4-[2-[3-(trifluoromethyl)benzyl]-1-benzothiophen-7-yl]pyrimidine-2-carboxamide

4-[2-[3-(Trifluoromethyl)benzyl]-1-benzothiophen-7-yl]pyrimidine-2-carboxylic acid obtained in Reference Example 366, and glycinamide hydrochloride were used in the same manner as in Working Example 196 to obtain the titled compound in solid form. Yield. 49%, melting point: 241-242° C. (ethyl acetate-hexane)

¹H-NMR (DMSO-d₆) δ: 4.04 (2H, d, J=5.3 Hz), 4.44 (2H, s), 7.27 (1H, br s), 7.35 (1H, s), 7.52-7.66 (4H, m), 7.68-7.81 (2H, m), 8.04 (1H, d, J=7.6 Hz), 8.33 (1H, d, J=7.6 Hz), 8.49 (1H, d, J=5.7 Hz), 8.90 (1H, t, J=5.3 Hz), 9.08 (1H, d, J=5.7 Hz).

### Working Example 334

### 5-Fluoro-N-(2-hydroxyethyl)-2-[2-[3-(trifluoromethyl)benzyl]-1-benzothiophen-7-yl]pyridine-4-carboxamide

2-Bromo-5-fluoro-N-(2-hydroxyethyl)pyridine-4-carboxamide obtained in Reference Example 367, and 4,4,5,5-tetramethyl-2-[2-[3-(trifluoromethyl)benzyl]-1-benzothiophene-7-yl]-1,3,2-dioxaborolane obtained in Reference Example 193 were used in the same manner as in Working Example 332 to obtain the titled compound in solid form. Yield. 37%, melting point: 110-111° C. (ethyl acetate-hexane)

¹H-NMR (DMSO-d₆) δ: 3.36 (2H, q, J=5.9 Hz), 3.55 (2H, q, J=5.9 Hz), 4.40 (2H, s), 4.80 (1H, t, J=5.9 Hz), 7.32 (1H, s), 7.47-7.69 (4H, m), 7.72 (1H, s), 7.90 (1H, d, J=7.2 Hz), 8.08 (1H, d, J=7.2 Hz), 8.33 (1H, d, J=5.3 Hz), 8.73 (1H, t, J=5.9 Hz), 8.83 (1H, s).

### Working Example 335

### N-(2-Hydroxyethyl)-3-[2-[3-(trifluoromethyl)phenoxy]-1,3-benzothiazol-7-yl]benzamide

3-[2-[3-(Trifluoromethyl)phenoxy]-1,3-benzothiazol-7-yl]benzoic acid obtained in Reference Example 370, and 2-aminoethanol were used in the same manner as in Working Example 3 to obtain the titled compound in solid form. Yield. 42%, melting point: 103-104° C. (ethyl acetate-hexane)

¹H-NMR (DMSO-d₆) δ: 3.32-3.42 (2H, m), 3.53 (2H, q, J=6.2 Hz), 4.73 (1H, t, J=6.2 Hz), 7.50-7.69 (3H, m), 7.72-7.89 (5H, m), 7.91-7.99 (2H, m), 8.10-8.17 (1H, m), 8.58 (1H, t, J=6.2 Hz).

### Working Example 336

### N-(2-Amino-2-oxoethyl)-3-[2-[3-(trifluoromethyl)phenoxy]-1,3-benzothiazol-7-yl]benzamide

3-[2-[3-(Trifluoromethyl)phenoxy]-1,3-benzothiazol-7-yl]benzoic acid obtained in Reference Example 370, and glycinamide hydrochloride were used in the same manner as in Working Example 196 to obtain the titled compound in solid form. Yield. 18%, melting point: 203-204° C. (ethyl acetate-hexane)

^{1H}-NMR (DMSO-d₆) δ: 3.84 (2H, d, J=6.0 Hz), 7.05 (1H, br s), 7.41 (1H, br s), 7.51-7.72 (3H, m), 7.72-7.90 (5H, m), 7.92-8.02 (2H, m), 8.17 (1H, s), 8.83 (1H, t, J=6.0 Hz).

### Working Example 337

### N-(2-Amino-2-oxoethyl)-3-fluoro-2-[2-[3-(trifluoromethyl)benzyl]-1-benzothiophen-7-yl]pyridine-4-carboxamide

A mixture of N-(2-amino-2-oxoethyl)-2-chloro-3-fluoropyridine-4-carboxamide (70.0 mg, 0.302 mmol) obtained in Reference Example 371, 4,4,5,5-tetramethyl-2-[2-[3-(trifluoromethyl)benzyl]-1-benzothiophen-7-yl]-1,3,2-dioxaborolane (115 mg, 0.275 mmol) obtained in Reference Example 193, and tetrakis(triphenylphosphine)palladium (0) (38.1 mg, 0.033 mmol) in 2 N sodium carbonate aqueous solution (0.412 mL)-1,2-dimethoxyethane (2.5 mL) was stirred for 4 hours at 90° C. in a nitrogen atmosphere. Water was poured into the reaction solution, and the mixture was extracted-with ethyl acetate. The extract was dried over anhydrous magnesium sulfate, and the solvent was distilled off at reduced pressure. The residue was purified by silica gel column chromatography (ethyl acetate:hexane=4:1) and recrystallized from hexane and ethyl acetate to give 71.9 mg of the titled compound (yield 54%) in solid form. Melting point: 169-170° C.

¹H-NMR (DMSO-d₆) δ: 3.88 (2H, d, J=5.7 Hz), 4.40 (2H, s), 7.14 (1H, br s), 7.34 (1H, s), 7.44 (1H, br s), 7.47-7.69 (5H, m), 7.72 (1H, s), 7.82-7.98 (2H, m), 8.67 (1H, d, J=4.9 Hz), 8.85 (1H, t, J=5.7 Hz).

### Working Example 338

### N-(2-Amino-2-oxoethyl)-3-[2-[3-(trfl uoromethyl)benzyl]-1,3-benzothiazol-7-yl]benzamide

3-[2-[3-(Trifluoromethyl)benzyl]-1,3-benzothiazol-7-yl]benzoic acid obtained in Reference Example 374, and glycinamide hydrochloride were used in the same manner as in Working Example 196 to obtain the titled compound in solid form. Yield. 15%, melting point: 139-140° C. (ethyl acetate-hexane)

¹H-NMR (DMSO-d₆) δ: 3.83 (2H, d, J=5.7 Hz), 4.64 (2H, s), 7.05 (1H, br s), 7.39 (1H, br s), 7.55-7.69 (5H, m), 7.70-7.77 (1H, m), 7.78-7.88 (2H, m), 7.91-8.06 (2H, m), 8.17 (1H, s), 8.81 (1H, t, J=5.5 Hz).

### Working Example 339

### N-(2-Hydroxyethyl)-3-[2-[3-(trifluoromethyl)benzyl]-1,3-benzothiazol-7-yl]benzamide

3-[2-[3-(Trifluoromethyl)benzyl]-1,3-benzothiazol-7-yl]benzoic acid obtained in Reference Example 374, and 2-aminoethanol were used in the same manner as in Working Example 3 to obtain the titled compound in solid form. Yield. 36%, melting point: 94-95° C. (diethyl ether-hexane)

¹H-NMR (DMSO-d₆) δ: 3.29-3.40 (2H, m), 3.52 (2H, q, J=5.9 Hz), 4.64 (2H, s), 4.72 (1H, t, J=5.9 Hz), 7.54-7.69 (5H, m), 7.69-7.77 (1H, m), 7.78-7.86 (2H, m), 7.92 (1H, d, J=8.0 Hz), 7.98-8.05 (1H, m), 8.11-8.17 (1H, m), 8.56 (1H, t, J=5.9 Hz).

### Working Example 340

### N-(2-Methoxyethyl)-3-[2-[3-(trifluoromethyl)benzyl]-1,3-benzothiazol-7-yl]benzamide

3-[2-[3-(Trifluoromethyl)benzyl]-1,3-benzothiazol-7-yl]benzoic acid obtained in Reference Example 374, and 2-methoxyethylamine were used in the same manner as in Working Example 3 to obtain the titled compound in solid form. Yield. 37%, melting point: 101-102° C. (diethyl ether-hexane)

¹H-NMR (DMSO-d₆) δ: 3.26 (3H, s), 3.40-3.51 (4H, m), 4.64 (2H, s), 7.54-7.69 (5H, m), 7.70-7.76 (1H, m), 7.78-7.85 (2H, m), 7.88-7.95 (1H, m), 7.98-8.05 (1H, m), 8.11-8.16 (1H, m), 8.59-8.69 (1H, m).

### Working Example 341

### 2-Fluoro-N-(2-methoxyethyl)-3-[2-[3-(trifluoromethyl)benzyl]-1-benzothiophen-7-yl]benzamide

3-Bromo-2-fluoro-N-(2-methoxyethyl)benzamide obtained in Reference Example 375, and 4,4,5,5-tetramethyl-2-[2-[3-(trifluoromethyl)benzyl]-1 benzothiophene-7-yl]-1,3,2-dioxaborolane obtained in Reference Example 193 were used in the same manner as in Working Example 337 to obtain the titled compound in solid form. Yield. 47%, melting point: 94-95° C. (diethyl ether-hexane)

¹H-NMR (DMSO-d₆) δ: 3.25 (3H, s), 3.35-3.49 (4H, m), 4.37 (2H, s), 7.28-7.41 (3H, m), 7.47 (1H, t, J=7.8 Hz), 7.52-7.73 (6H, m), 7.85 (1H, d, J=7.2 Hz), 8.41 (1H, br s).

### Working Example 342

### N-(2-Amino-2-oxoethyl)-2-fluoro-3-[2-[3-(trifluoromethyl)benzyl]-1-benzothiophen-7-yl]benzamide

N-(2-Amino-2-oxoethyl)-3-bromo-2-fluorobenzamide obtained in Reference Example 376, and 4,4,5,5-tetramethyl-2-[2-[3-(trifluoromethyl)benzyl]-1-benzothiophene-7-yl]-1,3,2-dioxaborolane obtained in Reference Example 193 were used in the same manner as in Working Example 337 to obtain the titled compound in solid form. Yield. 29%, melting point: 135-136° C. (diethyl ether-hexane)

¹H-NMR (DMSO-d₆) δ: 3.83 (2H, d, J=5.7 Hz), 4.37 (2H, s), 7.09 (1H, br s), 7.28-7.73 (10H, m), 7.73-7.82 (1H, m), 7.85 (1H, d, J=7.2 Hz), 8.39-8.49 (1H, m).

### Working Example 343

### 3-Fluoro-N-(2-methoxyethyl)-2-[2-[3-(trifluoromethyl)benzyl]-1-benzothiophen-7-yl]pyridine-4-carboxamide

2-Chloro-3-fluoro-N-(2-methoxyethyl)pyridine-4-carboxamide obtained in Reference Example 377, and 4,4,5,5-tetramethyl-2-[2-[3-(trifluoromethyl)benzyl]-1-benzothiophene-7-yl]-1,3,2-dioxaborolane obtained in Reference Example 193 were used in the same manner as in Working Example 337 to obtain the titled compound in solid form. Yield. 62%, melting point: 94-95° C. (diethyl ether-hexane)

¹H-NMR (DMSO-d₆) δ: 3.28 (3H, s), 3.40-3.50 (4H, m), 4.39 (2H, s), 7.34 (1H, s), 7.47-7.69 (5H, m), 7.71 (1H, s), 7.84-7.96 (2H, m), 8.65 (1H, d, J=3.8 Hz), 8.73-8.83 (1H, m).

### Working Example 344

### N-(2-Amino-2-oxoethyl)-3-[2-(3-chloro-5-fluorobenzyl)-1-benzothiophen-7-yl]-2-fluorobenzamide

N-(2-Amino-2-oxoethyl)-3-bromo-2-fluorobenzamide obtained in Reference Example 376, and 2-[2-(3-chloro-5-fluorobenzyl)-1-benzothiophene-7-yl]-4,4,5,5-tetramethyl-1,3,2-dioxaborolane obtained in Reference Example 192 were used in the same manner as in Working Example 337 to obtain the titled compound in solid form. Yield. 42%, melting point: 46-47° C. (ethyl acetate-hexane)

¹H-NMR (DMSO-d₆) δ: 3.84 (2H, d, J=5.7 Hz), 4.28 (2H, s), 7.09 (1H, br s), 7.16-7.24 (1H, m), 7.26-7.53 (7H, m), 7.64-7.73 (1H, m), 7.74-7.82 (1H, m), 7.86 (1H, d, J=6.8 Hz), 8.40-8.49 (1H, m).

### Working Example 345

### 3-[2-(3-Chloro-5-fluorobenzyl)-1-benzothiophen-7-yl]-2-fluoro-N-(2-methoxyethyl)benzamide

3-Bromo-2-fluoro-N-(2-methoxyethyl)benzamide obtained in Reference Example 375, and 2-[2-(3-chloro-5-fluorobenzyl)-1-benzothiophene-7-yl]-4,4,5,5-tetramethyl-1,3,2-dioxaborolane obtained in Reference Example 192 were used in the same manner as in Working Example 337 to obtain the titled compound in solid form. Yield. 30%, melting point: 124-125° C. (diethyl ether-hexane)

¹H-NMR (DMSO-d₆) δ: 3.26 (3H, s), 3.36-3.49 (4H, m), 4.28 (2H, s), 7.15-7.25 (1H, m), 7.26-7.42 (5H, m), 7.48 (1H, t, J=7.7 Hz), 7.60-7.71 (2H, m), 7.85 (1H, d, J=7.7 Hz), 8.37-8.48 (1H, m).

### Working Example 346

### N-(2-Amino-2-oxoethyl)-5-[2-(3-chloro-5-fluorobenzyl)-1-benzothiophen-7-yl]-2-fluorobenzamide

5-[2-(3-Chloro-5-fluorobenzyl)-1-benzothiophene-7-yl]-2-fluorobenzoic acid obtained in Reference Example 256, and glycinamide hydrochloride were used in the same manner as in Working Example 196 to obtain the titled compound in solid form. Yield. 56%, melting point: 129-130° C. (ethyl acetate-hexane)

¹H-NMR (DMSO-d₆) δ: 3.86 (2H, d, J=5.7 Hz), 4.30 (2H, s), 7.11 (1H, br s), 7.16-7.25 (1H, m), 7.26-7.34 (2H, m), 7.34-7.53 (5H, m), 7.78-7.88 (2H, m), 7.99 (1H, dd, J=7.0, 2.4 Hz), 8.40-8.51 (1H, m).

### Working Example 347

### N-(2-Amino-2-oxoethyl)-3-[2-(3-chloro-5-fluorobenzyl)-1-benzothiophen-7-yl]-4-fluorobenzamide

3-[2-(3-Chloro-5-fluorobenzyl)-1-benzothiophen-7-yl]-4-fluorobenzoic acid obtained in Reference Example 379, and glycinamide hydrochloride were used in the same manner as in Working Example 196 to obtain the titled compound in solid form. Yield. 61%, melting point: 159-160° C. (ethyl acetate-hexane)

¹H-NMR (DMSO-d₆) δ: 3.80 (2H, d, J=6.0 Hz), 4.28 (2H, s), 7.03 (1H, br s), 7.15-7.25 (1H, m), 7.26-7.43 (5H, m), 7.44-7.55 (2H, m), 7.87 (1H, d, J=7.2 Hz), 7.99-8.07 (1H, m), 8.10 (1H, dd, J=7.2, 2.3 Hz), 8.79 (1H, t, J=6.0 Hz).

### Working Example 348

### 5-[2-(3-Chloro-5-fluorobenzyl)-1-benzothiophen-7-yl]-2-fluoro-N-(2-methoxyethyl)benzamide

5-[2-(3-Chloro-5-fluorobenzyl)-1-benzothiophene-7-yl]-2-fluorobenzoic acid obtained in Reference Example 256, and 2-methoxyethylamine were used in the same manner as in Working Example 3 to obtain the titled compound in solid form. Yield. 64%, melting point: 84-85° C. (diethyl ether-hexane)

¹H-NMR (DMSO-d₆) δ: 3.27 (3H, s), 3.38-3.51 (4H, m), 4.30 (2H, s), 7.21 (1H, d, J=9.1 Hz), 7.27-7.34 (2H, m), 7.35-7.53 (4H, m), 7.76-7.88 (3H, m), 8.43 (1H, br s).

### Working Example 349

### N-(2-Amino-2-oxoethyl)-4-methyl-3-[2-[3-(trifluoromethyl)benzyl]-1-benzothiophen-7-yl]benzamide

4-Methyl-3-[2-[3-(trifluoromethyl)benzyl]-1-benzothiophen-7-yl]benzoic acid obtained in Reference Example 381, and glycinamide hydrochloride were used in the same manner as in Working Example 196 to obtain the titled compound in solid form. Yield. 76%, melting point: 165-166° C. (ethyl acetate-hexane)

¹H-NMR (DMSO-d₆) δ: 2.11 (3H, s), 3.77 (2H, d, J=5.7 Hz), 4.34 (2H, s), 7.01 (1H, s), 71.8 (1H, d, J=6.4 Hz), 7.34 (1H, s), 7.35 (1H, br s), 7.42-7.50 (2H, m), 7.50-7.66 (3H, m), 7.69 (1H, s), 7.76-7.91 (3H, m), 8.66 (1H, t, J=5.7 Hz).

### Working Example 350

### N-(2-Amino-2-oxoethyl)-2-methyl-5-[2-[3-(trifluoromethyl)benzyl]-1-benzothiophen-7-yl]benzamide

1 N sodium hydroxide aqueous solution (6.7 mL) was added at room temperature to a THF (10 mL)-methanol (5 mL) mixed solution of ethyl 2-methyl-5-[2-[3-(trifluoromethyl)benzyl]-1-benzothiophen-7-yl]benzoate (500 mg, 1.10 mmol) obtained in Reference Example 382, and the mixture was stirred over night at room temperature. Water was poured into the reaction solution, the pH was adjusted to between 2 and 3 with 1 N hydrochloric acid aqueous solution, and the mixture was extracted with ethyl acetate. The extract was washed with water and dried over anhydrous magnesium sulfate, and the solvent was distilled off at reduced pressure to give 328 mg of 2-methyl-5-[2-[3-(trifluoromethyl)benzyl]-1-benzothiophen-7-yl]benzoic acid (yield 70%) in solid form. An DMF (4.0 mL) solution of the resulting 2-methyl-5-[2-[3-(trifluoromethyl)benzyl]-1-benzothiophen-7-yl]benzoic acid (200 mg, 0.469 mmol), WSC (108 mg, 0.563 mmol), HOBt (76.1 mg, 0.563 mmol), glycinamide hydrochloride (57.0 mg, 1.87 mL), and N,N-diisopropylethylamine (0.090 mL, 0.516 mmol) was stirred for 3 hours at room temperature. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate) and recrystallized from ethyl acetate and hexane to give 101 mg of the titled compound (yield 45%) in solid form. Melting point: 143-144° C.

¹H-NMR (DMSO-d₆) δ 3.79 (2H, d, J=6.1 Hz), 4.38 (2H, s), 7.02 (1H, br s), 7.29-7.41 (4H, m), 7.42-7.49 (1H, m), 7.52-7.68 (5H, m), 7.70 (1H, s), 7.79 (1H, d, J=6.8 Hz), 8.46 (1H, t, J=6.1 Hz).

### Working Example 351

### 3-Chloro-5-[2-[3-(trifluoromethyl)benzyl]-1-benzothiophen-7-yl]benzamide

A mixture of the 4,4,5,5-tetramethyl-2-[2-[3-(trifluoromethyl)benzyl]-I -benzothiophen-7-yl]-1,3,2-dioxaborolane (300 mg, 0.808 mmol) obtained in Reference Example 193, 3-chloro-5-(dihydroxyboranyl)benzoic acid (178 mg, 0.889 mmol), and tetrakis(triphenylphosphine)palladium (0) (112 mg, 0.097 mmol) in 2 N sodium carbonate aqueous solution (1.2 mL)-1,2-dimethoxyethane (6.0 mL) was stirred for 4 hours at 90° C. in a nitrogen atmosphere. Water was poured into the reaction solution, and the mixture was extracted with ethyl acetate. The extract was dried over anhydrous magnesium sulfate, and the solvent was distilled off at reduced pressure. The residue was dissolved in DMF (7.2 mL). WSC (186 mg, 0.970 mmol) and HOBt (131 mg, 0.970 mmol) were added, and the mixture was stirred for 3 hours at room temperature. 28% aqueous ammonia (3 mL) was added dropwise to the reaction solution, and the mixture was stirred for 30 min at room temperature. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate:hexane=1:1) and recrystallized from hexane and ethyl acetate to give 48.9 mg of the titled compound (yield 14%) in solid form. Melting point: 139-140° C.

¹H-NMR (DMSO-d₆) δ: 4.40 (2H, s), 7.37 (1H, s), 7.41-7.69 (6H, m), 7.72 (1H, s), 7.81-7.88 (2H, m), 7.95-8.00 (1H, m), 8.09-8.15 (1H, m), 8.17 (1H, s).

### Working Example 352

### 3-Chloro-N-(2-methoxyethyl)-5 -[2-[3-(trifluoromethyl)benzyl]-1-benzothiophen-7-yl]benzamide

A mixture of the 4,4,5,5-tetramethyl-2-[2-[3-(trifluoromethyl)benzyl]-1-benzothiophen-7-yl]-1,3,2-dioxaborolane (300 mg, 0.808 mmol) obtained in Reference Example 193, 3-chloro-5-(dihydroxyboranyl)benzoic acid (178 mg, 0.889 mmol), and tetrakis(triphenylphosphine)palladium (0) (112 mg, 0.097 mmol) in 2 N sodium carbonate aqueous solution (1.2 mL)-1,2-dimethoxyethane (6.0 mL) was stirred for 4 hours at 90° C. in a nitrogen atmosphere. Water was poured into the reaction solution, and the mixture was extracted with ethyl acetate. The extract was dried over anhydrous magnesium sulfate, and the solvent was distilled off at reduced pressure. The residue was dissolved in DMF (7.2 mL). WSC (186 mg, 0.970 mmol), HOBt (131 mg, 0.970 mmol), and 2-methoxyethylamine (0.077 mL, 0.889 mmol) were added, and the mixture was stirred for 3 hours at room temperature. Water was poured into the reaction solution, and the mixture was extracted with ethyl acetate. The extract was washed with water and dried over anhydrous magnesium sulfate, and the solvent was distilled off at reduced pressure. The residue was purified by silica gel column chromatography (ethyl acetate:hexane=1:1) to give 116 mg of the titled compound (yield 28%) in the form of an oily substance.

¹H-NMR (DMSO-d₆) δ: 3.26 (3H, s), 3.39-3.51 (4H, m), 4.40 (2H, s), 7.38 (1H, s), 7.41-7.68 (5H, m), 7.72 (1H, s), 7.81-7.89 (2H, m), 7.96 (1H, t, J=1.9 Hz), 8.08-8.14 (1H, m), 8.68-8.83 (1H, m).

### Working Example 353

### N-(2-Amino-2-oxoethyl)-3-[6-fluoro-2-[3-(trifluoromethyl)benzyl]-1-benzothiophen-7-yl]benzamide

3-[6-Fluoro-2-[3-(trifluoromethyl)benzyl]-1-benzothiophen-7-yl]benzoic acid obtained in Reference Example 391, and glycinamide hydrochloride were used in the same manner as in Working Example 196 to obtain the titled compound in solid form. Yield. 81%, melting point: 130-131 ° C. (ethyl acetate-hexane)

¹H-NMR (DMSO-d₆) δ: 3.81 (2H, d, J=6.1 Hz), 4.34 (2H, s), 7.03 (1H, br s), 7.28-7.45 (3H, m), 7.49-7.76 (6H, m), 7.84 (1H, dd, J=8.7, 4.9 Hz), 7.99 (1H, d, J=7.6 Hz), 8.06 (1H, s), 8.77 (1H, t, J=6.1 Hz).

### Working Example 354

### 3-[6-Fluoro-2-[3-(trifluoromethyl)benzyl]-1-benzothiophen-7-yl]-N-(2-methoxyethyl)benzamide

3-[6-Fluoro-2-[3-(trifluoromethyl)benzyl]-1-benzothiophen-7-yl]benzoic acid obtained in Reference Example 391, and 2-methoxyethylamine were used in the same manner as in Working Example 3 to obtain the titled compound in solid form. Yield. 60%, melting point: 116-117° C. (diethyl ether-hexane)

¹H-NMR (DMSO-d₆) δ: 3.25 (3H, s), 3.37-3.50 (4H, m), 4.34 (2H, s), 7.30-7.44 (2H, m), 7.50-7.66 (4H, m), 7.67-7.74 (2H, m), 7.84 (1H, dd, J=8.7, 4.7 Hz), 7.92-7.99 (1H, m), 8.04 (1H, s), 8.56-8.67 (1H, m).

### Working Example 355

### 3-[6-Fluoro-2-[3-(trifluoromethyl)benzyl]-1-benzothiophen-7-yl]benzamide

3-[6-Fluoro-2-[3-(trifluoromethyl)benzyl]-1-benzothiophen-7-yl]benzoic acid obtained in Reference Example 391, and 28% aqueous ammonia were used in the same manner as in Working Example 197 to obtain the titled compound in solid form. Yield. 37%, melting point: 166-167° C. (ethyl acetate-hexane)

¹H-NMR (DMSO-d₆) δ: 4.34 (2H, s), 7.31-7.43 (2H, m), 7.47 (1H, s), 7.51-7.65 (4H, m), 7.67-7.74 (2H, m), 7.84 (1H, dd, J=8.8, 5.0 Hz), 7.94-8.01 (1H, m), 8.01-8.11 (2H, m).

### Working Example 356

### N-(2-Amino-2-oxoethyl)-2-methyl-6-[2-[3-(trifluoromethyl)benzyl]-1-benzothiophen-7-yl]pyridine-4-carboxamide

2-Methyl-6-[2-[3-(trifluoromethyl)benzyl]-1-benzothiophen-7-yl]pyridine-4-carboxylic acid obtained in Reference Example 393, and glycinamide hydrochloride were used in the same manner as in Working Example 196 to obtain the titled compound in solid form. Yield. 80%, melting point: 183-184° C. (ethyl acetate-hexane)

¹H-NMR (DMSO-d₆) δ: 2.67 (3H, s), 3.88 (2H, d, J=5.7 Hz), 4.41 (2H, s), 7.11 (1H, s), 7.26 (1H, s), 7.46 (1H, s), 7.49-7.71 (5H, m), 7.75 (1H, s), 7.89 (1H, d, J=7.2 Hz), 8.13 (1H, d, J=7.2 Hz), 8.42 (1H, s), 9.10 (1H, t, J=5.7 Hz).

### Working Example 357

### N-(2-Methoxyethyl)-2-methyl-6-[2-[3-(trifluoromethyl)benzyl]-1-benzothiophen-7-yl]pyridine-4-carboxamide

2-Methyl-6-[2-[3-(trifluoromethyl)benzyl]-1-benzothiophen-7-yl]pyridine-4-carboxylic acid obtained in Reference Example 393, and 2-methoxyethylamine were used in the same manner as in Working Example 3 to obtain the titled compound in solid form. Yield. 64%, melting point: 115-116° C. (diethylether-hexane)

¹H-NMR (DMSO-d₆) δ: 2.66 (3H, s), 3.29 (3H, s), 3.43-3.55 (4H, m), 4.41 (2H, s), 7.26 (1H, s), 7.48-7.70 (5H, m), 7.75 (1H, s), 7.89 (1H, d, J=7.2 Hz), 8.12 (1H, d, J=7.2 Hz), 8.37 (1H, s), 8.92 (1H, br s).

### Working Example 358

### N-(2-Amino-2-oxoethyl)-3-[6-[3-(trifluoromethyl)benzyl]thieno[3,2-d]pyrimidin-4-yl]benzamide

3-[6-[3-(Trifluoromethyl)benzyl]thieno[3,2-d]pyrimidin-4-yl]benzoic acid obtained in Reference Example 398, and glycinamide hydrochloride were used in the same manner as in Working Example 196 to obtain the titled compound in solid form. Yield. 66%, melting point: 205-206° C. (ethyl acetate-hexane)

¹H-NMR (DMSO-d₆) δ: 3.85 (2H, d, J=6.0 Hz), 4.57 (2H, s), 7.06 (1H, br s.), 7.42 (1H, br s), 7.54-7.68 (3H, m), 7.69-7.78 (2H, m), 7.82 (1H, s), 8.12 (1H, d, J=7.9 Hz), 8.25 (1H, d, J=7.9 Hz), 8.63 (1H, s), 8.89 (1H, t, J=6.0 Hz), 9.26 (1H, s).

### Working Example 359

### 3-[2-(3-Chloro-5-fluorobenzyl)-1-benzothiophen-7-yl]-N-(2-ethoxyethyl)benzamide

3-[2-(3-Chloro-5-fluorobenzyl)-1-benzothiophen-7-yl]benzoic acid obtained in Reference Example 399, and 2-ethoxyethanamine were used in the same manner as in Working Example 3, to obtain the titled compound in solid form. Yield: 73% melting point: 79-80° C. (diethyl ether-hexane).

¹H-NMR (CDCl₃) δ: 1.21 (3H, t, J=7.0 Hz), 3.42-3.73 (6H, m), 4.17 (2H, s), 6.59 (1H, br s), 6.84-6.92 (1H, m), 6.93-7.00 (1H, m), 7.06 (1H, s), 7.13 (1H, s), 7.30-7.37 (1H, m), 7.40-7.48 (1H, m), 7.51-7.60 (1H, m), 7.67-7.74 (1H, m), 7.78-7.86 (2H, m), 8.05-8.10 (1H, m).

### Working Example 360

### 3-[2-(3-Chloro-5-fluorobenzyl)-1-benzothiophen-7-yl]-N-[2-(1-methylethoxy)ethyl]benzamide

3-[2-(3-Chloro-5-fluorobenzyl)-1-benzothiophen-7-yl]benzoic acid obtained in Reference Example 399, and 2-(1-methylethoxy)ethanamine were used in the same manner as in Working Example 3, to obtain the titled compound in solid form. Yield: 72% melting point: 84-85 ° C. (diethyl ether-hexane).

¹H-NMR (CDCl₃) δ: 1.17 (6H, d, J=6.1 Hz), 3.57-3.71 (5H, m), 4.17 (2H, s) 6.59 (1H, br s), 6.84-6.92(1H, m), 6.93-7.00(1H, m), 7.06(1H, s), 7.13 (1H, s), 7.30-7.38 (1H, m), 7.40-7.48 (1H, m), 7.51-7.60 (1H, m), 7.67-7.74 (1H, m), 7.76-7.86 (2H, m), 8.04-8.11 (1H, m).

### Working Example 361

### 3-[2-(3-Chloro-5-fluorobenzyl)-1-benzothiophen-7-yl]-N-(2-hydroxypropyl)benzamide

3-[2-(3-Chloro-5-fluorobenzyl)-1-benzothiophen-7-yl]benzoic acid obtained in Reference Example 399, and 1-aminopropane-2-ol were used in the same manner as in Working Example 3 to give the titled compound in amorphous solid form. Yield. 66%.

¹H NMR (DMSO-d₆) δ: 1.07 (3H, d, J=6.4 Hz), 3.22 (2H, t, J=6.1 Hz), 3.73-3.84 (1H, m), 4.30 (2H, s), 4.73 (1H, d, J=4.5 Hz), 7.17-7.24 (1H, m), 7.25-7.34 (2H, m), 7.38 (1H, s), 7.39-7.44 (1H, m), 7.45-7.54 (1H, m), 7.56-7.64 (1H, m), 7.81 (1H, s), 7.83 (1H, s), 7.88- 7.96 (1H, m), 8.11-8.16 (1H, m), 8.51 (1H, t, J=5.7 Hz).

### Working Example 362

### 3-[2-(3-Chloro-5-fluorobenzyl)-1-benzothiophen-7-yl]-N-(2-hydroxy-2-methylpropyl) benzamide

3-[2-(3-Chloro-5-fluorobenzyl)-1-benzothiophen-7-yl]benzoic acid obtained in Reference Example 399, and 1-amino-2-methylpropane-2-ol were used in the same manner as in Working Example 3, to obtain the titled compound in solid form. Yield: 64% melting point: 123° C. (ethyl acetate-hexane).

¹H NMR (DMSO-d₆) δ: 1.10 (6H, s), 3.27 (2H, d, J=6.0 Hz), 4.29 (2H, s), 4.54 (1H, s), 7.16-7.25 (1H, m), 7.26-7.34 (2H, m), 7.38 (1H, s), 7.40-7.46 (1H, m), 7.46-7.54 (1H, m), 7.57-7.65 (1H, m), 7.82 (2H, dd, J=7.5, 0.8 Hz), 7.88-7.94 (1H, m), 8.14 (1H, t, J=1.5 Hz), 8.37 (1H, t, J=6.2 Hz).

### Working Example 363

### 3-[2-(3-Chloro-5-fluorobenzyl)-1-benzothiophen-7-yl]-N-(2-hydroxy-1-methylethyl) benzamide

3-[2-(3-Chloro-5-fluorobenzyl)-1-benzothiophen-7-yl]benzoic acid obtained in Reference Example 399, and 2-aminopropane-1-ol were used in the same manner as in Working 25 Example 3, to obtain the titled compound in amorphous solid form. Yield: 53%.

¹H NMR (DMSO-d₆) δ: 1.13 (3H, d, J=6.8 Hz), 3.42-3.49 (1H, m), 3.97-4.09 (2H, m), 4.29 (2H, s), 4.71 (1H, t, J=5.9 Hz), 7.17-7.25 (1H, m), 7.26-7.34 (2H, m), 7.35-7.45 (2H, m), 7.46-7.53 (1H, m), 7.56-7.66 (1H, m), 7.77-7.86 (2H, m), 7.88-7.94 (1H, m), 8.11 (1H, t, J=1.5 Hz), 8.19 (1H, d, J=8.0 Hz).

### Working Example 364

### 3-[2-(3-Chloro-5-fluorobenzyl)-1-benzothiophen-7-yl]-N-(2-hydroxy-1,1-dimethylethyl) benzamide

3-[2-(3-Chloro-5-fluorobenzyl)-1-benzothiophen-7-yl]benzoic acid obtained in Reference Example 399, and 2-amino-2-methylpropane-1-ol were used in the same manner as in Working Example 3, to obtain the titled compound in solid form. Yield: 34% melting point: 149-150° C. (ethyl acetate-hexane).

¹H NMR (DMSO-d₆) δ: 1.31 (6H, s), 3.52 (2H, d, J=6.1 Hz), 4.30 (2H, s), 4.87 (1H, t, J=6.1 Hz), 7.17-7.24 (1H, m), 7.26-7.34 (2H, m), 7.35-7.44 (2H, m), 7.45-7.53 (1H, m), 7.54-7.62 (1H, m), 7.64 (1H, s), 7.76-7.89 (3H, m), 8.01-8.06 (1H, m).

### Working Example 365

### 3-[2-(3-Chloro-5-fluorobenzyl)-1-benzothiophen-7-yl]-N-(2,3-dihydroxypropyl)benzamide

3-[2-(3-Chloro-5-fluorobenzyl)-1-benzothiophen-7-yl]benzoic acid obtained in Reference Example 399, and 3-aminopropane-1,2-diol were used in the same manner as in Working Example 3, to obtain the titled compound in amorphous solid form. Yield: 86%.

¹H NMR (DMSO-d₆) δ: 3.16-3.28 (1H, m), 3.35-3.48 (3H, m), 3.60-3.72 (1H, m), 4.30 (2H, s), 4.56 (1H, t, J=5.9 Hz), 4.80 (1H, d, J=4.9 Hz), 7.21 (1H, d, J=9.5 Hz), 7.25-7.34 (2H, m), 7.35-7.45 (2H, m), 7.46-7.54 (1H, m), 7.56-7.66 (1H, m), 7.82 (2H, d, J=7.6 Hz), 7.92 (1H, d, J=8.0 Hz), 8.13 (1H, s), 8.51 (1H, t, J=5.5 Hz).

### Working Example 366

### 3-[2-(3-Chloro-5-fluorobenzyl)-1-benzothiophen-7-yl]-N-(tetrahydro-2H-pyran-4-yl) benzamide

An DMF (4 mL) mixture of 3-[2-(3-chloro-5-fluorobenzyl)-1-benzothiophen-7-yl]benzoic acid (200 mg, 0.504 mmol) obtained in Reference Example 399, tetrahydro-2H-pyran-4-amine hydrochloride (76 mg, 0.55 mmol), diisopropylethylamine (96 μL, 0.55 mmol), WSC (115 mg, 0.600 mmol), and HOBt (81 mg, 0.60 mmol) was stirred for 16 hours at room temperature. The addition of water to the reaction solution was followed by extraction with ethyl acetate. The extract was washed with water and dried over magnesium sulfate, and the solvent was distilled off at reduced pressure. The residue was purified by silica gel column chromatography (hexane:ethyl acetate) and by HPLC, and was recrystallized from hexane and ethyl acetate to give 130 mg of the titled compound (yield 54%) in solid form. Melting point: 127-128° C. (ethyl acetate-hexane).

¹H NMR (DMSO-d₆) δ: 1.49-1.65 (2H, m), 1.71-1.82 (2H, m), 3.39 (2H, td, J=11.6, 1.7 Hz), 3.83-3.93 (2H, m), 3.94-4.10 (1H, m), 4.29 (2H, s), 7.21 (1H, dt, J=9.7, 1.8 Hz), 7.27-7.33 (2H, m), 7.36-7.43 (2H, m), 7.46-7.53 (1H, m), 7.57-7.64 (1H, m), 7.79-7.85 (2H, m), 7.88-7.94 (1H, m), 8.12 (1H, t, J=1.5 Hz), 8.39 (1H, d, J=8.0 Hz).

### Working Example 367

### N-[(1R)-2-Amino-1-methyl-2-oxoethyl]-3-[2-(3-chloro-5-fluorobenzyl)-1-benzothiophen-7-yl]benzamide

A DMF (4 mL) mixture of 3-[2-(3-chloro-5-fluorobenzyl)-1-benzothiophen-7-yl]benzoic acid (200 mg, 0.504 mmol) obtained in Reference Example 399, D-alaninamide hydrochloride (69 mg, 0.55 mmol), diisopropylethylamine (96 μL, 0.55 mmol), WSC (115 mg, 0.600 mmol), and HOBt (81 mg, 0.599 mmol) was stirred for 2 hours at room temperature. The addition of water to the reaction solution was followed by extraction with ethyl acetate. The extract was washed with water and dried over magnesium sulfate, and the solvent was distilled off at reduced pressure. The residue was purified by silica gel column chromatography (ethyl acetate) to give 170 mg of the titled compound (yield 73%) in the form of amorphous solids.

¹H NMR (DMSO-d₆) δ: 1.33 (3H, d, J=7.2 Hz), 4.30 (2H, s), 4.43 (1H, quint, J=7.2 Hz), 6.98 (1H, br s), 7.21 (1H, d, J=9.5 Hz), 7.26-7.34 (2H, m), 7.37 (2H, s), 7.40-7.45 (1H, m), 7.48 (1H, d, J=7.6 Hz), 7.57-7.66(1H, m), 7.78-7.87 (2H, m), 7.96 (1H, d, J=7.6 Hz), 8.16 (1H, s), 8.55 (1H, d, J=7.6 Hz).

### Working Example 368

### N-[(1S)-2-Amino-1-methyl-2-oxoethyl]-3-[2-(3-chloro-5-fluorobenzyl)-1-benzothiophen-7-yl]benzamide

3-[2-(3-Chloro-5-fluorobenzyl)-1-benzothiophen-7-yl]benzoic acid obtained in Reference Example 399, and L-alaninamide hydrochloride were used in the same manner as in Working Example 367, to obtain the titled compound in amorphous solid form. Yield: 89%.

¹H NMR (DMSO-d₆) δ: 1.34 (3H, d, J=7.2 Hz), 4.30 (2H, s), 4.44 (1H, quin, J=7.2 Hz), 6.98 (1H, br s), 7.17-7.55 (7H, m), 7.57-7.66 (1H, m), 7.80-7.89 (2H, m), 7.96 (1H, d, J=7.6 Hz), 8.16 (1H, s), 8.55 (1H, d, J=7.6 Hz).

### Working Example 369

### 3-[2-(3-Chloro-5-fluorobenzyl)-1-benzothiophen-7-yl]-N-(2-methoxy-2-methylpropyl) benzamide

A DMF (4 mL) mixture of 3-[2-(3-chloro-5-fluorobenzyl)-1-benzothiophen-7-yl]benzoic acid (200 mg, 0.504 mmol) obtained in Reference Example 399, 2-methoxy-2-methylpropane-1-amine oxalate hemihydrate (202 mg, 0.54 mmol), diisopropylethylamine (300 μL, 1.72 mmol), WSC (115 mg, 0.600 mmol), and HOBt (81 mg, 0.60 mmol) was stirred for 2 hours at room temperature. The addition of water to the reaction solution was followed by extraction with ethyl acetate. The extract was washed with water and dried over magnesium sulfate, and the solvent was distilled off at reduced pressure. The residue was purified by silica gel column chromatography (hexane:ethyl acetate) to give 40 mg of the titled compound (yield 17%) in solid form. Melting point: 89-90° C. (diethyl ether-hexane).

¹H NMR (DMSO-d₆) δ: 1.11 (6H, s), 3.14 (3H, s), 3.30-3.37 (2H, m), 4.30 (2H, s), 7.20 (1H, dt, J=9.7, 1.7 Hz), 7.25-7.34 (2H, m), 7.36-7.45 (2H, m), 7.46-7.54 (1H, m), 7.56-7.66 (1H, m), 7.77-7.86 (2H, m), 7.86-7.94 (1H, m), 8.13 (1H, t, J=1.7 Hz), 8.37 (1H, t, J=6.0 Hz).

### Working Example 370

### 3-[2-(3-Chloro-5-fluorobenzyl)-4-fluoro-1-benzothiophen-7-yl]-N-(2-hydroxyethyl) benzamide

WSC (291 mg, 1.52 mmol) was added while cooled on ice to a DMF (6.0 mL) mixed solution of 3-[2-(3-chloro-5-fluorobenzyl)-4-fluoro-1-benzothiophen-7-yl]benzoic acid (420 mg, 1.01 mmol) obtained in Reference Example 402, aminoethanol (93 mg, 1.52 mmol), HOBt (233 mg, 1.52 mmol), and triethylamine (204 mg, 2.02 mmol), and the mixture was then heated to room temperature and stirred for 15 hours. The reaction solution was diluted with water and extracted with ethyl acetate. The extract was washed with saturated brine and dried over anhydrous magnesium sulfate, and the solvent was then distilled off at reduced pressure. The residue was purified by silica gel column chromatography (hexane-ethyl acetate=15:85−0:100) and crystallized using hexane-ethyl acetate to give 280 mg of the titled compound (yield 61%) in the form of colorless crystals. Melting point: 114-119° C.

¹H-NMR (CDCl₃) δ: 2.43 (3H, t, J=5.1 Hz), 3.61-3.72 (2H, s), 3.81-3.92 (2H, m), 4.18 (2H, s), 6.64 (1H, br s), 6.85-6.94 (1H, m), 6.95-7.03 (1H, m), 7.04-7.18 (2H, m), 7.21-7.35 (2H, m), 7.50-7.60 (1H, m), 7.74-7.85 (2H, m), 8.00-8.07 (1H, m).

### Working Example 371

### 3- [2-(3-Chloro-5-fluorobenzyl)-4-fluoro-1-benzothiophen-7-yl]-N-(2-methoxyethyl) benzamide

3-[2-(3-Chloro-5-fluorobenzyl)-4-fluoro-1-benzothiophen-7-yl]benzoic acid (420 mg, 1.01 mmol) obtained in Reference Example 402, and 2-methoxyethylamine (114 mg, 1.52 mmol) were used in the same manner as in Working Example 370 to obtain the titled compound. Yield: 42%, colorless solid. melting point: 90-110° C. (hexane-ethyl acetate).

¹H-NMR (CDCl₃) δ: 3.38 (3H, s), 3.537-3.62 (2H, s), 3.63-3.72 (2H, m), 4.18 (2H, s), 6.54 (1H, br s), 6.85-6.92 (1H, m), 6.94-7.01 (1H, m), 7.04-7.16 (2H, m), 7.21 -7.33 (2H, m), 7.54 (1H, t, J=7.8 Hz), 7.73-7.83 (2H, m), 8.00-8.05 (1H, m).

### Working Example 372

### N-(2-Amino-2-oxoethyl)-3-[2-(3-chloro-5-fluorobenzyl)-4-fluoro- 1 -benzothiophen-7-yl]-5-fluorobenzamide

3-[2-(3-Chloro-5-fluorobenzyl)-4-fluoro-1-benzothiophen-7-yl]-5-fluorobenzoic acid (130 mg, obtained in Reference Example 406.300 mmol), and glycinamide hydrochloride (50 mg, 0.450 mmol) were used, in the same manner as in Working Example 196 to obtain the titled compound. Yield: 24%, colorless solid, melting point: 192-194° C. (hexane-ethyl acetate).

¹H-NMR (CDCl₃) δ: 3.82 (2H, d, J=6.0 Hz), 4.34 (2H, s), 7.08 (1H, br s), 7.20-7.41 (4H, m), 7.42 (1H, br s), 7.45-7.54 (2H, m), 7.65-7.81 (2H, m), 7.99-8.03 (1H, m), 8.92 (1H, t, J=6.0 Hz).

### Working Example 373

### N-(2-Amino-2-oxoethyl)-5-[2-(3-chloro-5-fluorobenzyl)-4-fluoro-1-benzothiophen-7-yl]-2 -fluorobenzamide

5-[2-(3-Chloro-5-fluorobenzyl)-4-fluoro-1-benzothiophen-7-yl]-2-fluorobenzoic acid (315 mg, 0 obtained in Reference Example 411.728 mmol), and glycinamide hydrochloride (121 mg, 1.09 mmol) were used, in the same manner as in Working Example 196 to obtain the titled compound. Yield: 73%, colorless solid, melting point: 148-150° C. (hexane-ethyl acetate).

¹H-NMR (CDCl₃) δ: 4.18 (2H, s), 4.20-4.25 (2H, s), 5.50 (1H, br s), 6.04 (1H, br s), 6.84-6.92 (1H, m), 6.94-7.01 (1H, m), 7.04-7.08 (1H, m), 7.10 (1H, dd, J=8.1, 9.6 Hz), 7.21-7.31 (3H, m), 7.41-7.55 (1H, m), 7.76 (1H, ddd, J=2.7, 5.1, 8.7 Hz), 8.33 (1H, dd, J=2.7, 8.7 Hz).

### Working Example 374

### N-(2-Amino-2-oxoethyl)-5-[2-(3-chloro-5-fluorobenzyl)-4-fluoro-1-benzothiophen-7-yl]-2-fluorobenzamide

A DMF (5.0 mL) mixture of 5-[2-(3-chloro-5-fluorobenzyl)-4-fluoro-1-benzothiophen-7-yl]-2-fluorobenzoic acid (315 mg, 0.728 mmol) obtained in Reference Example 411, WSC (209 mg, 1.09 mmol), and HOBt ammonia complex (166 mg, 1.09 mmol) was stirred for 15 hours. The reaction solution was diluted with water and extracted with ethyl acetate. The extract was washed with saturated brine and dried over anhydrous magnesium sulfate, and the solvent was then distilled off at reduced pressure. The residue was purified by silica gel column chromatography (hexane-ethyl acetate=92:8-65:35) and crystallized using hexane-ethyl acetate to give 240 mg of the titled compound (yield 76%) in the form of colorless crystals. Melting point: 157-159° C.

¹H-NMR(CDCl₃) δ: 4.18 (2H, s), 5.90 (1H, brs),6.70(1H, brs), 6.84-6.92 (6H, m), 6.94-7.01 (1H, m), 7.04-7.14 (2H, m), 7.21-7.31 (3H, m), 7.78 (1H, ddd, J=2.7, 5.1, 8.7 Hz), 8.37 (1H, dd, J=2.4, 7.5 Hz).

### Working Example 375

### 3-[2-(2,3-Difluorobenzyl)-1-benzothiophen-7-yl]-N-(2-methoxyethyl)benzamide

A 2 M sodium carbonate aqueous solution (1.1 mL)-1,2-dimethoxyethane (5.5 mL) mixture of 3-[2-(bromomethyl)-1-benzothiophen-7-yl]-N-(2-methoxyethyl)benzamide (223 mg, 0.55 mmol) obtained in Reference Example 187, (2,3-difluorophenyl)boronic acid (105 mg, 0.66 mmol), and tetrakistriphenylphosphine palladium (0) (26 mg, 0.022 mmol) was heated to reflux over night in a nitrogen atmosphere. The reaction solution was diluted with saturated brine and ethyl acetate, and was filtered using celite. The resulting filtrate was extracted with ethyl acetate. The combined organic layers were dried over anhydrous sodium sulfate and then concentrated at reduced pressure, the residue was purified by silica gel column chromatography (hexane-ethyl acetate 70:30→34:66), and the resulting crystals were recrystallized from hexane-diethyl ether-ethyl acetate to give 182 mg of the titled compound (yield 75%). Melting point: 77-78° C.

¹H-NMR (CDCl₃) δ: 3.39 (3H, m), 3.54-3.63 (2H, m), 3.64-3.74 (2H, m), 4.27 (2H, s), 6.53 (1H, br s), 6.98-7.11 (3H, m), 7.13 (1H, s), 7.28-7.35 (1H, m), 7.42 (1H, t, J=1.5 Hz), 7.50-7.59 (1H, m), 7.68 (1H, dd, J=8.0, 0.8 Hz), 7.80 (1H, t, J=1.5 Hz), 7.83 (1H, s), 8.04 (1H, t, J=1.6 Hz).

### Working Example 376

### 3-[2-(2,4-Difluorobenzyl)-1-benzothiophen-7-yl]-N-(2-methoxyethyl)benzamide

3-[2-(Bromomethyl)-1-benzothiophene-7-yl]-N-(2-methoxyethyl)benzamide obtained in Reference Example 187, and (2,4-difluorophenyl)boronic acid were used in the same manner as in Working Example 375 to obtain the titled compound. Yield: 74%, melting point: 94-95° C. (hexane-diethyl ether-ethyl acetate-).

¹H-NMR (CDCl₃) δ: 3.39 (3H, m), 3.53-3.61 (2H, m), 3.63-3.73 (2H, m), 4.20 (2H, s), 6.53 (1H, br s), 6.77-6.87 (2H, m), 7.10 (1H, s), 7.17-7.25 (1H, m), 7.28-7.34 (1H, m), 7.38-7.46 (1H, m), 7.50-7.59 (1H, m), 7.68 (1H, dd, J=8.0, 1.1 Hz), 7.77-7.86 (2H, m), 8.04 (1H, t, J=1.6 Hz).

### Working Example 377

### 3-[2-(2,5-Difluorobenzyl)-1-benzothiophen-7-yl]-N-(2-methoxyethyl)benzamide

3-[2-(Bromomethyl)-1-benzothiophene-7-yl]-N-(2-methoxyethyl)benzamide obtained in Reference Example 187, and (2,5-difluorophenyl)boronic acid were used in the same manner as in Working Example 375 to obtain the titled compound. Yield: 69%, melting point: 92-94° C. (hexane-diethyl ether-ethyl acetate-).

¹H-NMR (CDCl₃) δ: 3.38 (3H, m), 3.53-3.63 (2H, m), 3.63-3.74 (2H, m), 4.22 (2H, s), 6.55 (1H, br s), 6.84-7.08 (3H, m), 7.14 (1H, s), 7.30-7.35 (1H, m), 7.39-7.49 (1H, m), 7.50-7.59 (1H, m), 7.69 (1H, dd, J=8.0, 1.1 Hz), 7.77-7.87 (2H, m), 8.05 (1H, t, J=1.6 Hz).

### Working Example 378

### 3-[2-(4-Chloro-3-fluorobenzyl)-1-benzothiophen-7-yl]-N-(2-methoxyethyl)benzamide

3-[2-(Bromomethyl)-1-benzothiophene-7-yl]-N-(2-methoxyethyl)benzamide obtained in Reference Example 187, and (4-chloro-3-fluorophenyl)boronic acid were used in the same manner as in Working Example 375 to obtain the titled compound. Yield: 79%. Oily substance.

¹H-NMR (CDCl₃) δ: 3.35-3.41 (3H, m), 3.52-3.62 (2H, m), 3.64-3.73 (2H, m), 4.18 (2H, s), 6.54 (1H, br s), 6.98-7.03 (1H, m), 7.05 (1H, dd, J=9.6, 1.9 Hz), 7.12 (1H, t, J=1.0 Hz), 7.28-7.36 (2H, m), 7.40-7.47 (1H, m), 7.50-7.58 (1H, m), 7.69 (1H, dd, J=8.0, 1.1 Hz), 7.77-7.85 (2H, m), 8.05 (1H, t, J=1.6 Hz).

### Working Example 379

### 3-[2-(3,5-Difluorobenzyl)-1-benzothiophen-7-yl]-N-(2-methoxyethyl)benzamide

3-[2-(Bromomethyl)-1-benzothiophene-7-yl]-N-(2-methoxyethyl)benzamide obtained in Reference Example 187, and (3,5-difluorophenyl)boronic acid were used in the same manner as in Working Example 375 to obtain the titled compound. Yield: 59%, melting point: 96-97° C. (hexane-diethyl ether-ethyl acetate).

¹H-NMR (CDCl₃) δ: 3.33-3.43 (3H, m), 3.53-3.62 (2H, m), 3.63-3.73 (2H, m), 4.19 (2H, s), 6.54 (1H, br s), 6.68 (1H, tt, J=8.9, 2.3 Hz), 6.74-6.85 (2H, m), 7.14 (1H, s), 7.33 (1H, dd, J=7.4, 1.1 Hz), 7.40-7.48 (1H, m), 7.51-7.59 (1H, m), 7.70 (1H, dd, J=8.0, 1.1 Hz), 7.77-7.86 (2H, m), 8.05 (1H, t, J=1.8 Hz).

### Working Example 380

### 3-[2-(3,5-Dichlorobenzyl)-1-benzothiophen-7-yl]-N-(2-methoxyethyl)benzamide

3-[2-(Bromomethyl)-1-benzothiophene-7-yl]-N-(2-methoxyethyl)benzamide obtained in Reference Example 187, and (3,5-dichlorophenyl)boronic acid were used in the same manner as in Working Example 375 to obtain the titled compound. Yield: 35%, melting point: 109-110° C. (hexane-diethyl ether-ethyl acetate).

¹H-NMR (CDCl₃) δ: 3.38 (3H, s), 3.54-3.62 (2H, m), 3.64-3.73 (2H, m), 4.17 (2H, s), 6.54 (1H, br s), 7.11-7.18 (3H, m), 7.24 (1H, t, J=1.8 Hz), 7.31-7.38 (1H, m), 7.44 (1H, t, J=7.7 Hz), 7.50-7.59 (1H, m), 7.71 (1H, dd, J=7.8, 1.0 Hz), 7.77-7.86 (2H, m), 8.05 (1H, t, J=1.6 Hz).

### Working Example 381

### N-(2-Cyanoethyl)-3-[2-(4-fluoro-3-methoxybenzyl)-1-benzothiophen-7-yl]benzamide

Ethyl 3-[2-(4-fluoro-3-methoxybenzyl)-1-benzothiophen-7-yl]benzoate obtained in Reference Example 413, and 3-aminopropanenitrile were used in the same manner as in Working Example 162 to obtain the titled compound. Yield: 60%, melting point: 158-159° C. (hexane-ethyl acetate).

¹H-NMR (CDCl₃) δ: 2.77 (2H, t, J=6.2 Hz), 3.75 (2H, q, J=6.3 Hz), 3.85 (3H, s), 4.17 (2H, s), 6.59 (1H, br s), 6.77-6.83 (1H, m), 6.86 (1H, dd, J=8.1, 2.1 Hz), 7.01 (1H, dd, J=11.3, 8.2 Hz), 7.02 (1H, s), 7.29-7.33 (1H, m), 7.43 (1H, t, J=7.6 Hz), 7.56 (1H, t, J=7.7 Hz), 7.69 (1H, dd, J=8.0, 0.8 Hz), 7.77-7.89 (2H, m), 8.07 (1H, t, J=1.9 Hz).

### Working Example 382

### 3-[2-(4-Fluoro-3-methoxybenzyl)-1-benzothiophen-7-yl]-N-(2-methoxyethyl)benzamide

Ethyl 3-[2-(4-flubro-3-methoxybenzyl)-1-benzothiophen-7-yl]benzoate obtained in Reference Example 413, and 2-methoxyethylamine were used in the same manner as in Working Example 162 to obtain the titled compound. Yield: 62%, melting point: 70-71° C. (hexane-diethyl ether).

¹H-NMR (CDCl₃) δ: 3.38 (3H, s), 3.54-3.62 (2H, m), 3.64-3.72 (2H, m), 3.85 (3H, s), 4.17 (2H, s), 6.54 (1H, br s), 6.77-6.83 (1H, m), 6.86 (1H, dd, J=8.1, 2.1 Hz), 7.01 (1H, dd, J=11.0, 8.2 Hz), 7.09 (1H, s), 7.28-7.34 (1H, m), 7.38-7.46 (1H, m), 7.50-7.58 (1H, m), 7.68 (1H, dd, J=7.7, 1.1 Hz), 7.76-7.86 (2H, m), 8.06 (1H, t, J=1.6 Hz).

### Working Example 383

### 3-[2-(2,3-Dihydro-1-benzofuran-5-ylmethyl)-1-benzothiophen-7-yl]-N-(2-methoxyethyl) benzamide

Ethyl 3-[2-(2,3-dihydro-1-benzofuran-5-ylmethyl)-1-benzothiophen-7-yl]benzoate obtained in Reference Example 414, and 2-methoxyethylamine were used in the same manner as in Working Example 162 to obtain the titled compound. Yield: 70%, melting point: 79-80° C. (hexane-diethyl ether).

¹H-NMR (CDCl₃) δ: 3.17 (2H, t, J=8.8 Hz), 3.38 (3H, s), 3.53-3.61 (2H, m), 3.64-3.72 (2H, m), 4.14 (2H, s), 4.54 (2H, t, J=8.7 Hz), 6.54 (1H, br s), 6.71 (1H, d, J=8.2 Hz), 6.97-7.05 (1H, m), 7.09 (2H, s), 7.29 (1H, d, J=7.1 Hz), 7.41 (1H, t, J=7.6 Hz), 7.53 (1H, t, J=7.7 Hz), 7.67 (1H, d, J=8.0 Hz), 7.76-7.85 (2H, m), 8.04 (1H, s).

### Working Example 384

### 3-[2-(3-Cyanobenzyl)-1-benzothiophen-7-yl]-N-(2-methoxyethyl)benzamide

Ethyl 3-[2-(3-cyanobenzyl)-1-benzothiophen-7-yl]benzoate obtained in Reference Example 415, and 2-methoxyethylamine were used in the same manner as in Working Example 162 to obtain the titled compound. Yield: 65%, melting point: 86-92° C. (hexane-ethyl acetate).

¹H-NMR (CDCl₃) δ: 3.38 (3H, s), 3.53-3.61 (2H, m), 3.63-3.73 (2H, m), 4.25 (2H, s), 6.55 (1H, br s), 7.13 (1H, s), 7.31-7.37 (1H, m), 7.38-7.48 (2H, m), 7.49-7.59 (4H, m), 7.70 (1H, dd, J=8.0, 1.1 Hz), 7.77-7.85 (2H, m), 8.03-8.08 (1H, m).

### Working Example 385

### 3-[2-[(5-Chlorothiophen-2-yl)methyl]-1-benzothiophen-7-yl]-N-(2-methoxyethyl)benzamide

Ethyl 3-[2-[(5-chlorothiophen-2-yl)methyl]-1-benzothiophen-7-yl]benzoate obtained in Reference Example 416, and 2-methoxyethylamine were used in the same manner as in Working Example 162 to obtain the titled compound. Yield: 58%, melting point: 104-105° C. (hexane-ethyl acetate).

¹H-NMR (CDCl₃) δ: 3.39 (3H, s), 3.55-3.61 (2H, m), 3.64-3.72 (2H, m), 4.31 (2H, s), 6.54 (1H, br s), 6.68-6.71 (1H, m), 6.73-6.75 (1H, m), 7.18 (1H, t, J=1.0 Hz), 7.31-7.35 (1H, m), 7.44 (1H, t, J=7.6Hz), 7.52-7.59 (1H, m), 7.70 (1H, dd, J=7.8, 1.2 Hz), 7.79-7.85 (2H, m), 8.06 (1H, t, J=1.6 Hz).

### Working Example 386

### 3-[2-[3-(Dimethylamino)benzyl]-1-benzothiophen-7-yl]-N-(2-methoxyethyl)benzamide

Ethyl 3-[2-[3-(dimethylamino)benzyl]-1-benzothiophen-7-yl]benzoate obtained in Reference Example 417, and 2-methoxyethylamine were used in the same manner as in Working Example 162 to obtain the titled compound. Yield: 54%, melting point: 92-93° C. (hexane-diethyl ether-ethyl acetate-).

¹H-NMR (CDCl₃) δ: 2.93 (6H, s), 3.38 (3H, s), 3.53-3.62 (2H, m), 3.63-3.74 (2H, m), 4.17 (2H, s), 6.54 (1H, br s), 6.58-6.69 (3H, m), 7.10 (1H, s), 7.17 (1H, t, J=7.8 Hz), 7.27-7.32 (1H, m), 7.36-7.45 (1H, m), 7.48-7.58 (1H, m), 7.66 (1H, d, J=7.7 Hz), 7.77-7.86 (2H, m), 8.03 (1H, t, J=1.8 Hz).

### Working Example 387

### N-(2-Amino-2-oxoethyl)-3-[2-[3-(dimethylamino)benzyl]-1-benzothiophen-7-yl]benzamide

5 Ethyl 3-[2-[3-(dimethylamino)benzyl]-1-benzothiophen-7-yl]benzoate obtained in Reference Example 417, and glycinamide hydrochloride were used in the same manner as in Working Example 150 to obtain the titled compound. Yield: 85%, melting point: 149-151° C. (hexane-ethyl acetate).

¹H-NMR (CDCl₃) δ: 2.93 (6H, s), 4.17 (2H, s), 4.19 (2H, d, J=4.9 Hz), 5.46 (1H, br s), 6.07 (1H, br s), 6.59-6.68 (3H, m), 6.96 (1H, br s), 7.10 (1H, s), 7.13-7.22 (1H, m), 7.28 (1H, dd, J=7.3, 1.2 Hz), 7.39 (1H, t, J=7.7 Hz), 7.50-7.58 (1H, m), 7.66 (1H, dd, J=8.0, 1.1 Hz), 7.80-7.90 (2H, m), 8.09 (1H, t, J=1.5 Hz).

### Working Example 388

### 3-[2-[3-(Hydroxymethyl)benzyl]-1-benzothiophen-7-yl]-N-(2-methoxyethyl)benzamide

Ethyl 3-[2-[3-(hydroxymethyl)benzyl]-1-benzothiophen-7-yl]benzoate obtained in Reference Example 418, and 2-methoxyethylamine were used in the same manner as in Working Example 162 to obtain the titled compound. Yield: 75%. Oily substance.

¹H-NMR (CDCl₃) δ: 1.85 (1H, t, J=6.0 Hz), 3.37 (3H, s), 3.52-3.61 (2H, m), 3.67 (2H, q, J=5.0 Hz), 4.22 (2H, s), 4.67 (2H, d, J=5.5 Hz), 6.55 (1H, br s), 7.11 (1H, d, J=1.1 Hz), 7.18-7.25 (2H, m), 7.27-7.35 (3H, m), 7.41 (1H, td, J=7.6, 0.8 Hz), 7.49-7.57 (1H, m), 7.67 (1H, d, J=7.7 Hz), 7.77-7.83 (2H, m), 8.05 (1H, t, J=1.6 Hz).

### Working Example 389

### N-(2-Amino-2-oxoethyl)-3-[2-[3-(hydroxymethyl)benzyl]-1-benzothiophen-7-yl]benzamide

Ethyl 3-[2-[3-(hydroxymethyl)benzyl]-1-benzothiophen-7-yl]benzoate obtained in Reference Example 418, and glycinamide hydrochloride were used in the same manner as in Working Example 150 to obtain the titled compound. Yield: 61%, melting point: 155-156° C. (hexane-ethyl acetate).

¹H-NMR (DMSO-d₆) δ: 3.80 (2H, d, J=5.8 Hz), 4.23 (2H, s), 4.44 (2H, d, J=5.5 Hz), 5.13 (1H, d, J=5.5Hz), 7.03 (1H, s), 7.15 (2H, m, J=5.8Hz), 7.21-7.28 (2H, m), 7.30 (1H, s), 7.35-7.42 (2H, m), 7.42-7.51 (1H, m), 7.59 (1H, d, J=7.7 Hz), 7.75-7.85 (2H, m), 7.91 (1H, d, J=7.7 Hz), 8.09-8.18 (1H, m), 8.72-8.86 (1H, m).

### Working Example 390

### 3-[2-(3-Chloro-5-fluorobenzyl)-3-methyl-1-benzothiophen-7-yl]-N-(2-hydroxyethyl) benzamide

Ethyl 3-[2-(3-chloro-5-fluorobenzyl)-3-methyl- I -benzothiophen-7-yl]benzoate obtained in Reference Example 419, and 2-aminoethanol were used in the same manner as in Working Example 162 to obtain the titled compound. Yield: 68%, melting point: 146-147° C. (hexane-ethyl acetate).

¹H-NMR (CDCl₃) δ: 2.40 (3H, s), 2.42-2.52 (1H, m), 3.60-3.71 (2H, m), 3.85 (2H, q, J=4.9 Hz), 4.16 (2H, s), 6.62 (1H, br s), 6.67-6.84 (1H, m), 6.92 (1H, dt, J=8.5, 2.1 Hz), 6.99 (1H, s), 7.32-7.39 (1H, m), 7.45-7.58 (2H, m), 7.67 (1H, dd, J=8.0, 1.1 Hz), 7.78-7.86 (2H, m), 8.05 (1H, t, J=1.6Hz).

### Working Example 391

### 3-[2-(3-Chloro-5-fluorobenzyl)-3-methyl-1-benzothiophen-7-yl]-N-(2-methoxyethyl) benzamide

Ethyl 3-[2-(3-chloro-5-fluorobenzyl)-3-methyl-1-benzothiophen-7-yl]benzoate obtained in Reference Example 419, and 2-methoxyethylamine were used in the same manner as in Working Example 162 to obtain the titled compound. Yield: 64%, melting point: 132-133° C. (hexane-ethyl acetate).

¹H-NMR (CDCl₃) δ: 2.40 (3H, s), 3.35-3.41 (3H, m), 3.52-3.62 (2H, m), 3.63-3.74 (2H, m), 4.17 (2H, s), 6.55 (1H, br s), 6.78-6.85 (1H, m), 6.93 (1H, dt, J=8.7, 2.0Hz), 7.00 (1H, s), 7.38 (1H, dd, J=7.3, 1.0 Hz), 7.46-7.59 (2H, m), 7.68 (1H, dd, J=7.8, 1.0 Hz), 7.78-(7.86 (2H, m), 8.05 (1H, t, J=1.6 Hz).

### Working Example 392

### N-(2-Methoxyethyl)-3-[2-([[4-(trifluoromethyl)pyridin-2-yl]oxy]methyl)-1-benzothiophen-7-yl]benzamide

Ethyl 3-[2-([[4-(trifluoromethyl)pyridin-2-yl]oxy]methyl)-1-benzothiophen-7-yl]benzoate obtained in Reference Example 420, and 2-methoxyethylamine were used in the same manner as in Working Example 162 to obtain the titled compound. Yield: 70%, melting point: 186-187° C. (hexane-ethyl acetate).

¹H-NMR (CDCl₃) δ: 3.39 (3H, s), 3.55-3.62 (2H, m), 3.64-3.73 (2H, m), 5.36 (2H, s), 6.29 (1H, dd, J=7.1, 1.9 Hz), 6.55 (1H, br s), 6.87 (1H, s), 7.35-7.40 (1H, m), 7.42-7.60 (4H, m), 7.75 (1H, dd, J=7.8, 1.0 Hz), 7.81 (2H, dd, J=7.4, 1.6 Hz), 8.04 (1H, t, J=1.6 Hz).

### Working Example 393

### N-(2-Methoxyethyl)-3-[2-([[6-(trifluoromethyl)pyridin-2-yl]oxy]methyl)-1-benzothiophen-7-yl]benzamide

3-[2-(Hydroxymethyl)-1-benzothiophene-7-yl]-N-(2-methoxyethyl)benzamide obtained in Reference Example 178, and 6-(trifluoromethyl)pyridine-2-ol were used in the same manner as in Reference Example 420 to obtain the titled compound. Yield: 63%. Oily substance.

¹H-NMR (CDCl₃) δ: 3.39 (3H, s), 3.54-3.62 (2H, m), 3.64-3.74 (2H, m), 5.67 (2H, s), 6.56 (1H, br s), 6.95 (1H, d, J=8.5 Hz), 7.28 (1H, d, J=7.4 Hz), 7.34-7.40 (1H, m), 7.45 (1H, d, J=7.7Hz), 7.49 (1H, s), 7.51-7.60(1H, m), 7.67-7.75 (1H, m), 7.77 (1H, dd, J=7.7, 1.1 Hz), 7.80-7.89 (2H, m), 8.07 (1H, t, J=1.6 Hz).

### Working Example 394

### N-(2-Methoxyethyl)-3-[2-([[2-(trifluoromethyl)pyridin-4-yl]oxy]methyl)-1-benzothiophen-7-yl]benzamide

3-[2-(Hydroxymethyl)-1-benzothiophene-7-yl]-N-(2-methoxyethyl)benzamide obtained in Reference Example 178, and 2-(trifluoromethyl)pyridine-4-ol were used in the same manner as in Reference Example 420 to obtain the titled compound. Yield: 42%. Oily substance.

1H-NMR (CDCl₃) δ: 3.39 (3H, s), 3.55-3.62 (2H, m), 3.64-3.74 (2H, m), 5.42 (2H, s), 6.57 (1H, br s), 7.05 (1H, dd, J=5.8, 2.5 Hz), 7.29 (1H, d, J=2.5 Hz), 7.39-7.43 (1H, m), 7.45 (1H, s), 7.46-7.53 (1H, m), 7.54-7.61 (1H, m), 7.75-7.89 (3H, m), 8.10 (1H, t, J=1.8 Hz), 8.56 (1H, t, J=5.8 Hz).

### Working Example 395

### 3-[2-[(2,3-Difluorophenoxy)methyl]-1-benzothiophen-7-yl]-N-(2-methoxyethyl)benzamide

3-[2-(Hydroxymethyl)-1-benzothiophene-7-yl]-N-(2-methoxyethyl)benzamide, and 2,3-difluorophenol obtained in Reference Example 178 were used in the same manner as in Reference Example 420 to obtain the titled compound. Yield: 84%. Oily substance.

¹H-NMR (CDCl₃) δ: 3.39 (3H, s), 3.55-3.64 (2H, m), 3.65-3.76 (2H, m), 5.39 (2H, s), 6.57 (1H, br s), 6.73-6.88 (2H, m), 6.89-7.00 (1H, m), 7.35-7.43 (1H, m), 7.43-7.51 (1H, m), 7.53-7.61 (1H, m), 7.76 (1H, dd, J=7.7, 1.1 Hz), 7.80-7.90 (2H, m), 8.08 (1H, t, J=1.6 Hz).

### Working Example 396

### N-(2-Amino-2-oxoethyl)-3-[2-[(3,5-dichlorophenoxy)methyl]-1-benzothiophen-7-yl]benzamide

N-(2-amino-2-oxoethyl)-3-[2-(hydroxymethyl)-1-benzothiophen-7-yl]benzamide obtained in Reference Example 178, and 3,5-dichlorophenol were used in the same manner as in Reference Example 420 to give the titled compound. Yield: 44%, melting point: 164-165° C. (hexane-ethyl acetate).

¹H-NMR (DMSO-d₆) δ: 3.83 (2H, d, J=5.8 Hz), 5.51 (2H, s), 7.04 (1H, s), 7.17 (3H, s), 7.40 (1H, br s), 7.45-7.73 (4H, m), 7.83-8.05 (3H, m), 8.19 (1H, s), 8.83 (1H, t, J=5.8 Hz).

### Working Example 397

### N-(2-Methoxyethyl)-3-(2-[2-[3-(trifluoromethyl)phenyl]ethyl]-1-benzothiophen-7-yl) benzamide

Ethyl 3-(2-[2-[3-(trifluoromethyl)phenyl]ethyl]-1-benzothiophen-7-yl)benzoate obtained in Reference Example 423, and 2-methoxyethylamine were used in the same manner as in Working Example 162 to obtain the titled compound. Yield: 88%. Oily substance.

¹H-NMR (CDCl₃) δ: 3.06-3.17 (2H, m), 3.18-3.28 (2H, m), 3.40 (3H, s), 3.55-3.63 (2H), m), 3.65-3.74 (2H, m), 6.57 (1H, br s), 7.06 (1H, s), 7.30-7.35 (1H, m), 7.35-7.51 (5H, m), 7.53-7.62 (1H, m), 7.76 (1H, d, J=8.0 Hz), 7.79-7.90 (2H, m), 8.10 (1H, s).

### Working Example 398

### N-(2-Amino-2-oxoethyl)-3-(2-[2-[3-(trifluoromethyl)phenyl]ethyl]-1-benzothiophen-7-yl) benzamide

Ethyl 3-(2-[2-[3-(trifluoromethyl)phenyl]ethyl]-1-benzothiophen-7-yl)benzoate obtained in Reference Example 423, and glycinamide hydrochloride were used in the same manner as in Working Example 150 to obtain the titled compound. Yield: 63%, melting point: 153-154° C. (hexane-ethyl acetate).

¹H-NMR (CDCl₃) δ: 3.05-3.15 (2H, m), 3.16-3.28 (2H, m), 4.22 (2H, d, J=4.9 Hz), 5.52 (1H, br s), 6.17 (1H, brs), 7.02-7.11 (2H, m), 7.28-7.34 (1H, m), 7.35-7.51 (5H, m), 7.53-7.62 (1H, m), 7.67 (1H, dd, J=8.0, 1.1 Hz), 7.82-7.92 (2H, m), 8.15 (1H, t, J=1.8 Hz).

### Working Example 399

### N-(2-Methoxyethyl)-3-[2-([[3-(trifluoromethyl)phenyl]amino]methyl)-1-benzothiophen-7-yl]benzamide

Ethyl 3-[2-([[3-(trifluoromethyl)phenyl]amino]methyl)-1-benzothiophen-7-yl]benzoate obtained in Reference Example 424, and 2-methoxyethylamine were used in the same manner as in Working Example 162 to obtain the titled compound. Yield: 57%. Oily substance.

¹H-NMR (CDCl₃) δ: 3.37 (3H, s), 3.53-3.63 (2H, m), 3.63-3.73 (2H, m), 4.35-4.47 (2H, m), 4.62 (2H, d, J=5.8 Hz), 6.56 (1H, br s), 6.80 (1H, dd, J=8.2, 2.5 Hz), 6.89 (1H, s), 6.93-7.01 (1H, m), 7.20-7.30 (1H, m), 7.30-7.37 (2H, m), 7.44 (1H, t, J=7.7Hz), 7.51-7.58 (1H, m), 7.71 (1H, d, J=8.0Hz), 7.78-7.88 (2H, m), 8.07 (1H, t, J=1.8 Hz).

### Working Example 400

### N-(2-Amino-2-oxoethyl)-3-[2-([[3-(trifluoromethyl)phenyl]amino]methyl)-1-benzothiophen-7-yl]benzamide

Ethyl 3-[2-([[3-(trifluoromethyl)phenyl]amino]methyl)-1-benzothiophen-7-yl]benzoate obtained in Reference Example 424, and glycinamide hydrochloride were used in the same manner as in Working Example 150 to obtain the titled compound. Yield: 59%. Oily substance.

¹H-NMR (CDCl₃) δ: 4.19 (2H, d, J=4.9 Hz), 4.39-4.50 (1H, m), 4.62 (2H, d, J=5.8 Hz), 5.51 (1H, br s), 6.10 (1H, br s), 6.76-6.85 (1H, m), 6.90 (1H, s), 6.96 (1H, d, J=7.4 Hz), 7.04 (1H, br s), 7.20-7.29 (1H, m), 7.30-7.38 (2H, m), 7.40-7.47 (1H, m), 7.52-7.60 (1H, m), 7.71 (1H, d, J=8.0Hz), 7.85 (2H, dd, J=7.7, 1.9Hz), 8.14 (1H, t, J=1.8 Hz).

### Working Example 401

### N-(2-Methoxyethyl)-3-[2-([methyl[3-(trifluoromethyl)phenyl]amino]methyl)-1-benzothiophen-7-yl]benzamide

Ethyl 3-[2-([methyl[3 -(trifluoromethyl)phenyl]amino]methyl)-1-benzothiophen-7-yl]benzoate obtained in Reference Example 425, and 2-methoxyethylamine were used in the same manner as in Working Example 162 to obtain the titled compound. Yield: 73%. Oily substance.

¹H-NMR (CDCl₃) δ: 3.11 (3H, s), 3.38 (3H, s), 3.54-3.62 (2H, m), 3.63-3.73 (2H, m), 4.76 (2H, s), 6.55 (1H, br s), 6.90-7.03 (3H, m), 7.21 (1H, s), 7.26-7.37 (2H, m), 7.43 (1H, t, J=7.5 Hz), 7.51-7.59 (1H, m), 7.69 (1H, dd, J=8.0, 1.1 Hz), 7.78-7.86 (2H, m), 8.05 (1H, t, J=1.5 Hz).

### Working Example 402

### 3-Fluoro-5-[3-fluoro-2-[3-(trifluoromethyl)benzyl]-1-benzothiophen-7-yl]-N-(2-methoxyethyl)benzamide

Ethyl 3-fluoro-5-[3-fluoro-2-[3-(trifluoromethyl)benzyl]-1-benzothiophen-7-yl]benzoate obtained in Reference Example 432, and 2-methoxyethylamine were used in the same manner as in Working Example 162 to obtain the titled compound. Yield: 70%, melting point: 109-110° C. (hexane-diethyl ether-ethyl acetate-).

¹H-NMR (CDCl₃) δ: 3.37 (3H, s), 3.55-3.60 (2H, m), 3.62-3.72 (2H, m), 4.26 (2H, s), 6.49 (1H, br s), 7.35-7.58 (8H, m), 7.73-7.78 (1H, m), 7.79 (1H, t, J=1.2 Hz).

### Working Example 403

### N-(2-Amino-2-oxoethyl)-3-fluoro-5-[3-fluoro-2-[3-(trifluoromethyl)benzyl]-1; -benzothiophen-7-yl]benzamide

Ethyl 3-fluoro-5-[3-fluoro-2-[3-(trifluoromethyl)benzyl]-1-benzothiophen-7-yl]benzoate obtained in Reference Example 432, and glycinamide hydrochloride were used in the same manner as in Working Example 150 to obtain the titled compound. Yield: 70%, melting point: 145-146° C. (hexane-ethyl acetate).

¹H-NMR (CDCl₃) δ: 4.17 (2H, d, J=4.7 Hz), 4.24 (2H, s), 5.47 (1H, br s), 5.90 (1H, br s), 6.95 (1H, br s), 7.32-7.61 (8H, m), 7.74 (1H, dd, J=8.1, 1.0 Hz), 7.83 (1H, t, J=1.2 Hz).

### Working Example 404

### 3-[3-Fluoro-2-[3-(trifluoromethyl)benzyl]-1-benzothiophen-7-yl]-N-(2-methoxyethyl) benzamide

Ethyl 3-[3-fluoro-2-[3-(trifluoromethyl)benzyl]-1-benzothiophen-7-yl]benzoate obtained in Reference Example 431, and 2-methoxyethylamine were used in the same manner as in Working Example 162 to obtain the titled compound. Yield: 75%, melting point: 115-116° C. (hexane-diethyl ether-ethyl acetate-).

¹H-NMR (CDCl₃) δ: 3.37 (3H, s), 3.52-3.62 (2H, m), 3.63-3.72 (2H, m), 4.24 (2H, s), 6.53 (1H, br s), 7.36 - 7.59 (7H, m), 7.70 - 7.85 (3H, m), 8.02 (1H, t, J=1.8 Hz).

### Working Example 405

### N-(2-Amino-2-oxoethyl)-3-[3-fluoro-2-[3-(trifluoromethyl)benzyl]-1-benzothiophen-7-yl]benzamide

Ethyl 3-[3-fluoro-2-[3-(trifluoromethyl)benzyl]-1-benzothiophen-7-yl]benzoate obtained in Reference Example 431, and glycinamide hydrochloride were used in the same manner as in Working Example 150 to obtain the titled compound. Yield: 57%, melting point: 127-128° C. C (hexane-ethyl acetate).

¹H-NMR (CDCl₃) δ: 4.19 (2H, d, J=4.9 Hz), 4.24 (2H, s), 5.46 (1H, br s), 5.97 (1H, br s), 6.95 (1H, br s), 7.34-7.61 (7H, m), 7.73 (1H, dd, J=8.0, 0.8 Hz), 7.85 (1H, dt, J=7.6, 1.3 Hz), 8.07 (1H, t, J=1.8 Hz).

### Working Example 406

### N-(2-Cyanoethyl)-3-[3-fluoro-2-[3-(trifluoromethyl)benzyl]-1-benzothiophen-7-yl]benzamide

Ethyl 3-[3-fluoro-2-[3-(trifluoromethyl)benzyl]-1-benzothiophen-7-yl]benzoate obtained in Reference Example 431, and 3-aminopropionitrile were used in the same manner as in Working Example 162 to obtain the titled compound. Yield: 80%, melting point: 66-67° C. (hexane-ethyl acetate).

¹H-NMR (CDCl₃) δ: 2.76 (2H, t, J=6.3 Hz), 3.73 (2H, q, J=6.1 Hz), 4.24 (2H, s), 6.57 (1H, br s), 7.32-7.63 (7H, m), 7.73 (1H, dd, J=8.0, 1.1 Hz), 7.80 (1H, dd, J=7.7, 1.9 Hz), 8.02 (1H, t, J=1.8Hz).

### Working Example 407

### N-(2-Hydroxyethyl)-3-(2-[hydroxy[3-(trifluoromethyl)phenyl]methyl]imidazo[1,2-a]pyridine-5-yl)benzamide

(5-Bromoimidazo[1,2-a]pyridin-2-yl)[3-(trifluoromethyl)phenyl]methanol obtained in Reference Example 434, and 3-ethoxycarbonylphenylboronic acid were used in the same manner as in Reference Example 10 to give ethyl 3-(2-[hydroxy[3-(trifluoromethyl)phenyl]methyl]imidazo[1,2-a]pyridine-5-yl)benzoate crude product. The resulting crude product of ethyl 3-(2-[hydroxy[3-(trifluoromethyl)phenyl]methyl]imidazo[1,2-a]pyridine-5-yl)benzoate was used in the same manner as in Reference Example 5 to give 3-(2-[hydroxy[3-(trifluoromethyl)phenyl]methyl]imidazo[1,2-a]pyridine-5-yl)benzoic acid crude product. The resulting crude product 3-(2-[hydroxy[3-(trifluoromethyl)phenyl]methyl]imidazo[1,2-a]pyridine-5-yl)benzoic acid, and 2-aminoethanol were used to obtain the titled compound. Yield: 38% ( calculated through 4 steps from 5-bromoimidazo[1,2-a]pyridine-2-carbaldehyde). melting point: 169° C. (ethanol-ethyl acetate-).

¹H-NMR (CDCl₃) δ: 2.39 (1H, br s), 3.51 (1H, d, J=4.4 Hz), 3.62-3.70 (2H, m), 3.81-3.90 (2H, m), 6.01 (1H, d, J=4.1 Hz), 6.70 (1H, br s), 6.74 (1H, dd, J=6.9, 1.1 Hz), 7.26 (1H, dd, J=9.1, 6.9 Hz), 7.37 (1H, s), 7.40-7.47 (1H, m), 7.49-7.54 (1H, m), 7.54-7.59 (1H, m), 7.59-7.63 (1H, m), 7.65 (1H, d, J=7.4 Hz), 7.69-7.76 (2H, m), 7.88-7.93 (1H, m), 7.98-8.00 (1H, m).

### Working Example 408

### N-(2-Cyanoethyl)-3-(2-[hydroxy[3-(trifluoromethyl)phenyl]methyl]imidazo[1,2-a]pyridine-5-yl)benzamide

(5-Bromoimidazo[1,2-a]pyridin-2-yl)[3-(trifluoromethyl)phenyl]methanol obtained in Reference Example 434, and 3-ethoxycarbonylphenylboronic acid were used in the same manner as in Reference Example 10 to give ethyl 3-(2-[hydroxy[3-(trifluoromethyl)phenyl]methyl]imidazo[1,2-a]pyridine-5-yl)benzoate crude product. The resulting crude product of ethyl 3-(2-[hydroxy[3-(trifluoromethyl)phenyl]methyl]imidazo[1,2-a]pyridine-5-yl)benzoate was used in the same manner as in Reference Example 5 to give is 3-(2-[hydroxy[3-(trifluoromethyl)phenyl]methyl]imidazo[1,2-a]pyridine-5-yl)benzoic acid, crude product. The resulting crude product of 3-(2-[hydroxy[3-(trifluoromethyl)phenyl]methyl]imidazo[1,2-a]pyridine-5-yl)benzoic acid, and 3-aminopropionitrile were used to obtain the titled compound. Yield: 52%, melting point: 223-224° C. (hexane-ethanol).

¹H-NMR (CDCl₃) δ: 2.77 (2H, t, J=6.0 Hz), 3.49 (1H, br s), 3.68-3.77 (2H, m), 6.01 (1H, d, J=4.1 Hz), 6.75 (1H, br s), 6.76 (1H, dd, J=6.9, 0.8Hz), 7.26-7.31 (1H, m), 7.38 (1H, s), 7.40-7.48 (1H, m), 7.49-7.54 (1H, m), 7.55-7.68 (3H, m), 7.73-7.78 (2H, m), 7.89-7.94 (1H, m), 8.00-8.04 (1H, m).

### Working Example 409

### N-(2-Cyanoethyl)-3-[2-[3-(trifluoromethyl)benzyl]imidazo[1,2-a]pyridine-5-yl]benzamide

Ethyl 3-[2-[3-(trifluoromethyl)benzyl]imidazo[1,2-a]pyridine-5-yl]benzoate obtained in Reference Example 437, and 3-aminopropionitrile were used in the same manner as in Working Example 162 to obtain the titled compound. Yield: 91%, melting point: 192-193° C. (hexane-ethyl acetate).

¹H-NMR (CDCl₃) δ: 2.78 (2H, t, J=6.3 Hz), 3.69-3.78 (2H, m), 4.13 (2H, s), 6.71 (1H, dd, J=6.9, 1.1 Hz), 6.87 (1H, brs), 7.23 (1H, dd, J=9.1,6.9Hz), 7.29 (1H, s), 7.33-7.40 (1H, m), 7.41-7.50 (2H, m), 7.52 (2H, s), 7.54-7.59 (1H, m), 7.59-7.66 (1H, m), 7.74-7.79 (1H, m), 7.88-7.93 (1H, m), 8.02 (1H, t, J=1.6 Hz).

### Working Example 410

### N-(2-Cyanoethyl)-3-[2-[3-(trifluoromethyl)benzyl]furo[2,3-c]pyridin-7-yl]benzamide

A mixture of (7-chlorofuro[2,3-c]pyridin-2-yl)[3-(trifluoromethyl)phenyl]methanol (143 mg, 0.436 mmol) obtained in Reference Example 443, triethylsilane (349 μL, 2.18 mmol), and trifluoroacetic acid (2.0 mL) was stirred for 4 hours at room temperature and was then stirred for 18 hours at 80° C. Triethylsilane (349 μL, 2.18 mmol) was added to the reaction solution, and the mixture was stirred for another 7 hours at 80° C. The reaction solution was diluted with ethyl acetate and saturated sodium bicarbonate aqueous solution, and was then extracted with ethyl acetate. The extract was washed with saturated brine and dried over sodium sulfate. The solvent was distilled off at reduced pressure, and the residue was purified by silica gel column chromatography (hexane-ethyl acetate 70:30→50:50) to give 7-chloro-2-[3-(trifluoromethyl)benzyl]furo[2,3-c]pyridine crude product (292 mg). A 2 M sodium carbonate aqueous solution (872 μL, 1.74 mmol)-1,2-dimethoxyethane (5.0 mL) mixture of the resulting 7-chloro-2-[3-(trifluoromethyl)benzyl]furo[2,3-c]pyridine crude product (292 mg), [3-[(2-cyanoethyl)carbamoyl]phenyl]boronic acid (114 mg, 0.52 mmol) obtained in Reference Example XX, and tetrakistriphenylphosphine palladium (0) (20 mg, 0.017 mmol) was heated to reflux for 4 hours in a nitrogen atmosphere. The reaction solution was diluted with saturated brine and ethyl acetate, and was extracted with ethyl acetate. The resulting organic layer was dried over anhydrous sodium sulfate and then concentrated at reduced pressure, and the residue was purified by silica gel column chromatography (hexane-ethyl acetate 70:30→50:50, ethyl acetate-methanol 100:0→95:5) (NH, ethyl acetate-methanol 100:0→95:5) and preparative HPLC to give the titled compound. Yield: 11%. White solids.

¹H-NMR (CDCl₃) δ: 2.79 (2H, t, J=6.3 Hz), 3.72-3.81 (2H, m), 4.30 (2H, s), 6.48 (1H, s), 6.72 (1H, br s), 7.41-7.68 (6H, m), 7.90-7.95 (1H, m), 8.47 (1H, t, J=5.2 Hz), 8.48-8.53 (1H, m), 8.71-8.74 (1H, m).

### Working Example 411

### N-(2-Hydroxyethyl)-3-[2-[3-(trifluoromethyl)benzyl]furo[2,3-c]pyridin-7-yl]benzamide

Ethyl 3-[2-[3-(trifluoromethyl)benzyl]furo[2,3-c]pyridin-7-yl]benzoate obtained in Reference Example 446, and 2-aminoethanol were used in the same manner as in Working Example 162 to obtain the titled compound. Yield: 28%, melting point: 168-169° C. (hexane-ethyl acetate).

¹H-NMR (CDCl₃) δ: 2.78-2.85 (1H, m), 3.63-3.72 (2H, m), 3.82-3.92 (2H, m), 4.28 (2H, s), 6.47 (1H, s), 6.83 (1H, br s), 7.42 (1H, d, J=5.2 Hz), 7.45-7.66 (5H, m), 7.90-7.95 (1H, m), 8.43-8.48 (2H, m), 8.69-8.72 (1H, m).

### Working Example 41.2

### N-(2-Cyanoethyl)-3-[2-[3-(trifluoromethyl)benzyl]furo[3,2-c]pyridin-7-yl]benzamide

3-[2-[3-(Trifluoromethyl)benzyl]furo[3,2-c]pyridin-7-yl]benzoic acid obtained in Reference Example 450, and 3-aminopropionitrile were used in the same manner as in Working Example 3 to obtain the titled compound. Yield: 64%, melting point: 152-154° C. (hexane-ethyl acetate).

¹H-NMR (CDCl₃) δ: 2.80 (2H, t, J=6.3 Hz), 3.76 (2H, q, J=6.0 Hz), 4.24 (2H, s), 6.51 (1H, s), 6.77 (1H, br s), 7.42-7.67 (5H, m), 7.81-7.87 (1H, m), 7.97 (1H, ddd, J=7.8, 1.8, 1.1 Hz), 8.25 (1H, t, J=1.5 Hz), 8.64 (1H, s), 8.80 (1H, s).

### Working Example 413

### N-(2-Hydroxyethyl)-3-[2-[3-(trifluoromethyl)benzyl]furo[3,2-c]pyridin-7-yl]benzamide

3-[2-[3-(Trifluoromethyl)benzyl]furo[3,2-c]pyridin-7-yl]benzoic acid obtained in Reference Example 450, and 2-aminoethanol were used in the same manner as in Working Example 3 to obtain the titled compound. Yield: 68%, melting point: 155-156° C. (hexane-ethyl acetate).

¹H-NMR (CDCl₃) δ: 2.89 (1H, br s), 3.64-3.73 (2H, m), 3.88 (2H, t, J=3.8 Hz), 4.22 (2H, s), 6.50 (1H, s), 6.80 (1H, br s), 7.44-7.64 (5H, m), 7.82 (1H, dq, J=7.7, 1.0 Hz), 7.91 (1H, dq, J=7.7, 1.0Hz), 8.21 (1H, t, J=1.8 Hz), 8.60 (1H, s), 8.77 (1H, s).

### Working Example 414

### N-(2-Cyanoethyl)-3-[2-[3-(trifluoromethyl)benzyl]furo[3,2-b]pyridin-7-yl]benzamide

3-[2-[3-(Trifluoromethyl)benzyl]furo[3,2-b]pyridin-7-yl]benzoic acid obtained in Reference Example 458, and 3-aminopropionitrile were used in the same manner as in Working Example 3 to obtain the titled compound. Yield: 70%, melting point: 164-165° C. (hexane-ethyl acetate).

¹H-NMR (CDCl₃) δ: 2.78 (2H, t, J=6.1 Hz), 3.75 (2H, q, J=6.2 Hz), 4.26 (2H, s), 6.60 (1H, br s), 6.70 (1H, s), 7.36 (1H, d, J=5.3 Hz), 7.43-7.68 (5H, m), 7.82-7.86 (1H, m), 8.02-8.08 (1H, m), 8.32 (1H, t, J=1.7Hz), 8.55 (1H, d, J=4.9Hz).

### Working Example 415

### N-(2-Hydroxyethyl)-3-[2-[3-(trifluoromethyl)benzyl]furo[3,2-b]pyridin-7-yl]benzamide

3-[2-[3-(Trifluoromethyl)benzyl]furo[3,2-b]pyridin-7-yl]benzoic acid obtained in Reference Example 458, and 2-aminoethanol were used in the same manner as in Working Example 3 to obtain the titled compound. Yield: 74%, melting point: 158-159° C. (hexane-ethyl acetate).

¹H-NMR (CDCl₃) δ: 2.38 (1H, t, J=4.9 Hz), 3.64-3.72 (2H, m), 3.87 (2H, t, J=5.2 Hz), 4.25 (2H, s), 6.64 (1H, br s), 6.70 (1H, s), 7.36 (1H, d, J=5.3 Hz), 7.43-7.65 (5H, m), 7.81-7.87 (1H, m), 8.00-8.06 (1H, m), 8.31 (1H, t, J=1.7 Hz), 8.54 (1H, d, J=5.3 Hz).

### Working Example 416

### N-(2-Methoxyethyl)-2-(2-[[3-(trifluoromethyl)phenoxy]methyl]-1-benzothiophen-7-yl)pyridine-4-carboxamide

Ethyl 2-(2-[[3-(trifluoromethyl)phenoxy]methyl]-1-benzothiophene-7-yl)pyridine-4-carboxylate obtained in Reference Example 461, and 2-methoxyethylamine were used in the same manner as in Working Example 162 to obtain the titled compound. Yield: 66%. Oily substance.

¹H-NMR (CDCl₃) δ: 3.42 (3H, s), 3.56-3.67 (2H, m), 3.67-3.77 (2H, m), 5.40 (2H, s), 6.66 (1H, br s), 7.15-7.25 (2H, m), 7.26-7.31 (1H, m), 7.35-7.46 (2H, m), 7.47-7.59 (2H, m), 7.86 (1H, d, J=7.7 Hz), 7.99 (1H, d, J=6.9 Hz), 8.33 (1H, s), 8.92 (1H, d, J=5.2 Hz).

### Working Example 417

### N-(2-Amino-2-oxoethyl)-2-(2-[[3-(trifluoromethyl)phenoxy]methyl]-1-benzothiophen-7-yl)pyridine-4-carboxamide

Ethyl 2-(2-[[3-(trifluoromethyl)phenoxy]methyl]-1-benzothiophene-7-yl)pyridine-4-carboxylate obtained in Reference Example 461, and glycinamide hydrochloride were used in the same manner as in Working Example 150 to obtain the titled compound. Yield: 58%, melting point: 185-186° C. (hexane-ethyl acetate).

¹H-NMR (DMSO-d₆) δ: 3.38 (2H, d, J=5.8 Hz), 5.56 (2H, s), 7.08-7.15 (1H, m), 7.27-7.34 (1H, m), 7.35-7.66 (6H, m), 7.78 (1H, dd, J=4.9,0.8Hz), 7.99 (1H, d, J=8.0 Hz), 8.22 (1H, d, J=7.7 Hz), 8.63 (1H, s), 8.91 (1H, d, J=4.9 Hz), 9.16-9.23 (1H, m).

### Working Example 418

### N-(2-Methoxyethyl)-4-(2-[[3-(trifluoromethyl)phenoxy]methyl]-1-benzothiophen-7-yl)benzamide

Ethyl 4-(2-[[3-(trifluoromethyl)phenoxy]methyl]-1-benzothiophen-7-yl)benzoate obtained in Reference Example 462, and 2-methoxyethylamine were used in the same manner as in Working Example 162 to obtain the titled compound. Yield: 96%. Oily substance.

¹H-NMR (CDCl₃) δ: 3.41 (3H, s), 3.55-3.64 (2H, m), 3.65-3.76 (2H, m), 5.35 (2H, s), 6.58 (1H, brs), 7.12-7.19 (1H, m), 7.21-7.25 (2H, m), 7.34-7.51 (4H, m), 7.73-7.84 (3H, m), 7.87-7.96 (2H, m).

### Working Example 419

### N-(2-Amino-2-oxoethyl)-4-(2-[[3-(trifluoromethyl)phenoxy]methyl]-1-benzothiophen-7-yl)benzamide

Ethyl 4-(2-[[3-(trifluoromethyl)phenoxy]methyl]-1-benzothiophen-7-yl)benzoate obtained in Reference Example 462, and glycinamide hydrochloride were used in the same manner as in Working Example 150 to obtain the titled compound. Yield: 75%, melting point: 164-165° C. (methanol-ethyl acetate).

¹H-NMR (CDCl₃) δ: 4.22 (2H, d, J=5.2 Hz), 5.34 (2H, s), 5.50 (1H, br s), 6.08 (1H, br s), 7.04 (1H, br s), 7.12-7.19 (1H, m), 7.21-7.25 (2H, m), 7.35-7.44 (3H, m), 7.43-7.51 (1H, m), 7.73-7.87 (3H, m), 7.96 (2H, d, J=8.0 Hz).

### Working Example 420

### 4-(2-[[3-(Trifluoromethyl)phenoxy]methyl]-1-benzothiophen-7-yl)benzamide

Ethyl 4-(2-[[3-(trifluoromethyl)phenoxy]methyl]-1-benzothiophen-7-yl)benzoate obtained in Reference Example 462, and aqueous ammonia were used in the same manner as in Working Example 157 to obtain the titled compound. Yield: 72%, melting point: 191-192° C. (hexane-ethyl acetate).

¹H-NMR (CDCl₃) δ: 5.36 (2H, s), 5.71 (1H, br s), 6.14 (1H, br s), 7.16 (1H, dd, J=8.5, 2.2 Hz), 7.21-7.26 (2H, m), 7.34-7.54 (4H, m), 7.74-7.86 (3H, m), 7.90-8.01 (2H, m),

### Working Example 421

### 3-Methoxy-N-(3-[2-[3-(trifluoromethyl)benzyl]-1-benzothiophen-7-yl]phenyl)propanamide

N-[3-[2-(bromomethyl)-1-benzothiophen-7-yl]phenyl]-3-methoxypropanamide obtained in Reference Example 464, and [3-(trifluoromethyl)phenyl]boronic acid were used in the same manner as in Working Example 375 to obtain the titled compound. Yield: 55%, melting point: 102-103° C.

¹H-NMR (CDCl₃) δ: 2.64 (2H, t, J=5.6 Hz), 3.42 (3H, s), 3.69-3.76 (2H, m), 4.26 (2H, s), 7.08 (1H, s), 7.27-7.32 (1H, m), 7.36-7.51 (6H, m), 7.53 (1H, s), 7.57-7.69 (2H, m), 7.74 (1H, s), 8.31 (1H, br s).

### Working Example 422

### 3-[2-(3-Chloro-5-fluorobenzyl)-1-benzothiophen-7-yl]-N-[2-(dimethylamino)ethyl]benzamide

3-[2(3-Chloro-5-fluorobenzyl)-1-benzothiophen-7-yl]benzoic acid obtained in Reference Example 399, and N,N-dimethylethane-1,2-diamine were used in the same manner as in Working Example 3 to obtain the titled compound. Yield: 53%, melting point: 126-127° C. (hexane-ethyl acetate).

¹H-NMR (CDCl₃) δ: 2.25 (6H, s), 2.52 (2H, t, J=5.9 Hz), 3.49-3.60 (2H, m), 4.18 (2H, s), 6.80-6.93 (1H, m), 6.97 (1H, dt, J=8.5, 2.1 Hz), 7.06 (1H, s), 7.14 (1H, s), 7.32-7.39 (1H, m), 7.41-7.49 (1H, m), 7.50-7.59 (1H, m), 7.71 (1H, dd, J=7.7, 1.1 Hz), 7.82 (2H, dd, J=7.7, 1.6 Hz), 8.07 (1H, t, J=1.8 Hz).

### Working Example 423

### tert-Butyl [2-[([3-[2-(3-chloro-5-fluorobenzyl)-1-benzothiophen-7-yl]phenyl]carbonyl)amino]ethyl]carbamate

3-[2-(3-Chloro-5-fluorobenzyl)-1-benzothiophen-7-yl]benzoic acid obtained in Reference Example 399, and tert-butyl (2-aminoethyl)carbamate were used in the same manner as in Working Example 3 to obtain the titled compound. Yield: 85%.

¹H-NMR (CDCl₃) δ: 1.38 (9H, s), 3.42 (2H, q, J=5.6 Hz), 3.59 (2H, q, J=5.4 Hz), 4.18 (2H, s), 6.85-6.93 (1H, m), 6.96 (1H, dt, J=8.4, 2.0 Hz), 7.06 (1H, s), 7.12 (1H, s), 7.19-7.31 (1H, m), 7.31-7.38 (1H, m), 7.39-7.48 (1H, m), 7.49-7.59 (1H, m), 7.69 (1H, dd, J=7.8, 1.0 Hz), 7.78-7.93 (2H, m), 8.10 (1H, s).

### Working Example 424

### N-(2-Aminoethyl)-3-[2-(3-chloro-5-fluorobenzyl)-1-benzothiophen-7-yl]benzamide hydrochloride

4 N hydrochloric acid-ethyl acetate solution (0.6 mL) was added to an ethyl acetate solution (7 mL) of tert-butyl [2-[([3-[2-(3-chloro-5-fluorobenzyl)-1-benzothiophen-7-yl]phenyl]carbonyl)amino]ethyl]carbamate (323 mg, 0.599 mmol) obtained in Working Example 423. The mixture was stirred for 1.5 hours at room temperature, 4 N hydrochloric acid-ethyl acetate solution (0.6 mL) was then added, and the mixture was stirred over night. The mixture was stirred for 1 hour at 50° C., hydrochloric acid-methanol solution (2.0 mL) was then added, and the mixture was stirred for another hour. The reaction solution was concentrated at reduced pressure, and the residue was recrystallized from hexane/ethyl acetate to give 282 mg of the titled compound (yield 59%). Melting point: 179-180° C. (hexane-ethyl acetate).

¹H-NMR (CDCl₃) δ: 3.14 (2H, br s), 3.59 (2H, br s), 3.86 (2H, s), 6.68 (1H, d, J=8.8 Hz), 6.76-6.90 (3H, m), 6.99-7.19 (3H, m), 7.34-7.59 (2H, m), 7.74 (1H, d, J=7.4 Hz), 8.00 (1H, s), 8.17 (2H, br s), 8.30 (1H, br s).

Table 4 shows the structures of the compounds obtained in Working Examples 253 through 424.

TABLE 4

Working Example 253

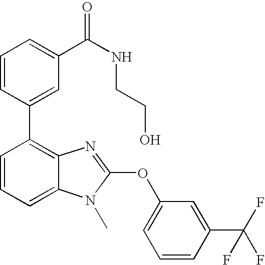

Working Example 254

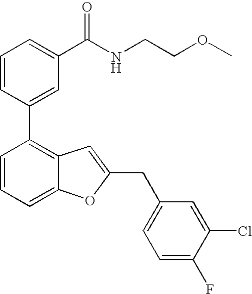

Working Example 255

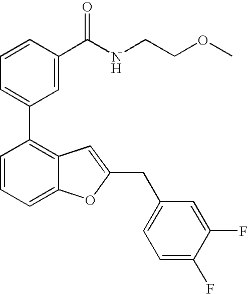

Working Example 256

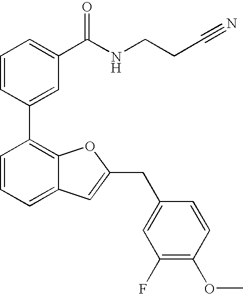

Working Example 257

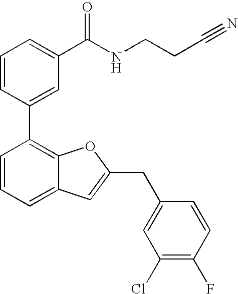

Working Example 258

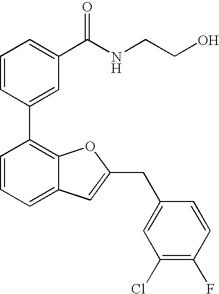

Working Example 259

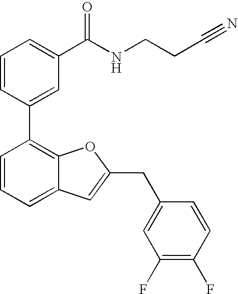

Working Example 260

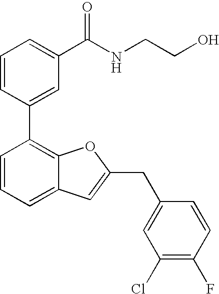

Working Example 261

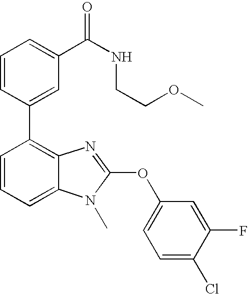

Working Example 262

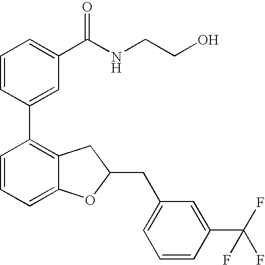

Working Example 263

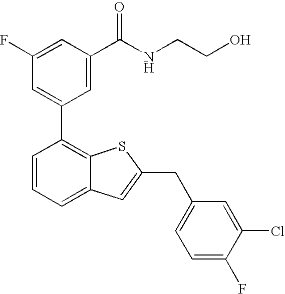

Working Example 264

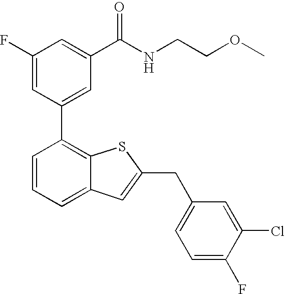

Working Example 265

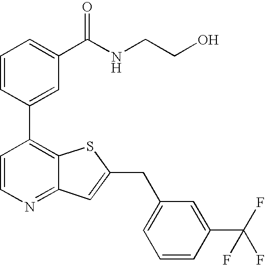

Working Example 266

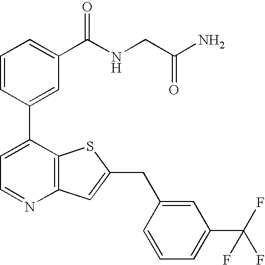

Working Example 267

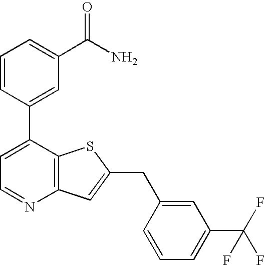

Working Example 268

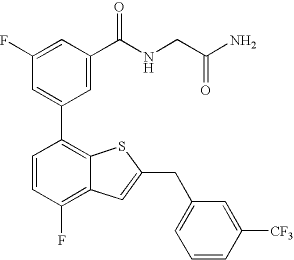

Working Example 269

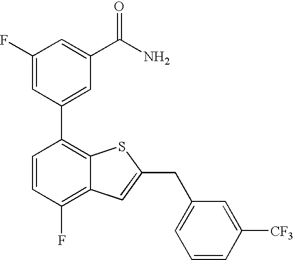

Working Example 270

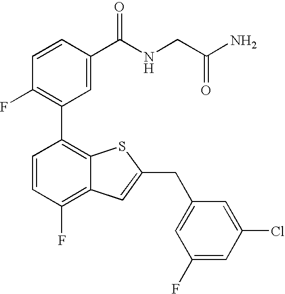

Working Example 271

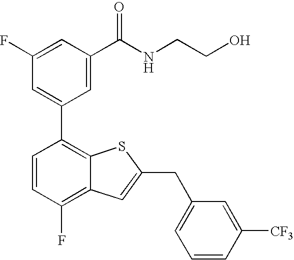

Working Example 272

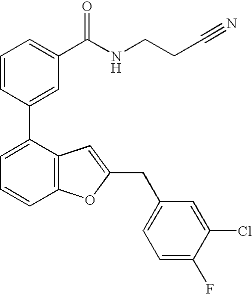

Working Example 273

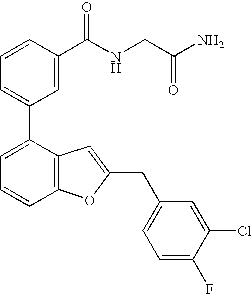

Working Example 274

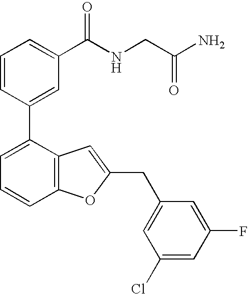

Working Example 275

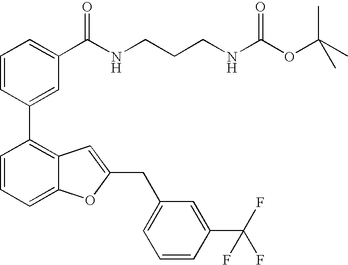

Working Example 276

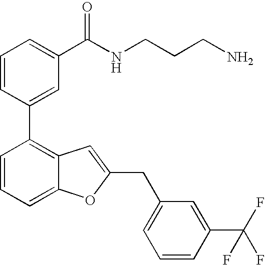

Working Example 277

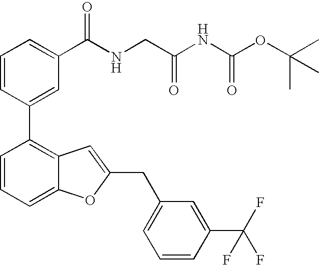

Working Example 278

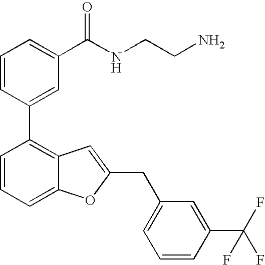

Working Example 279

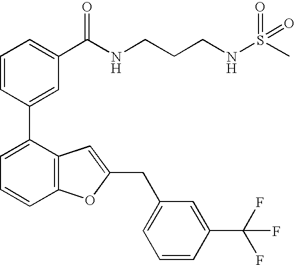

Working Example 280

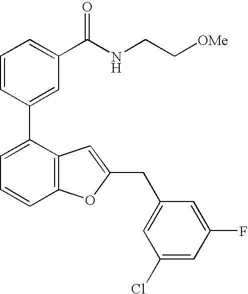

Working Example 281

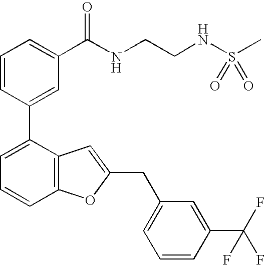

Working Example 282

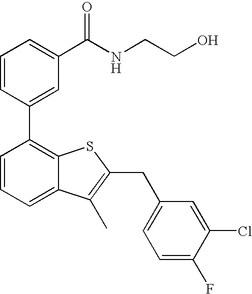

Working Example 283

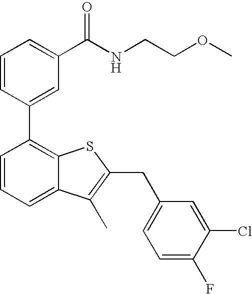

Working Example 284

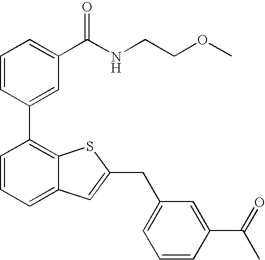

Working Example 285

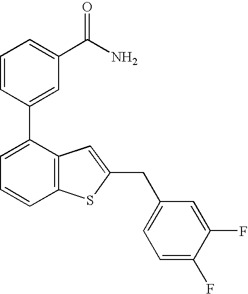

Working Example 286

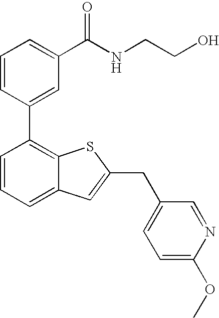

Working Example 287

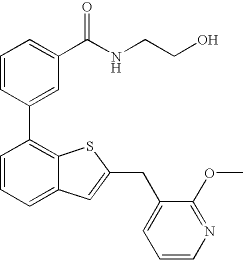

Working Example 288

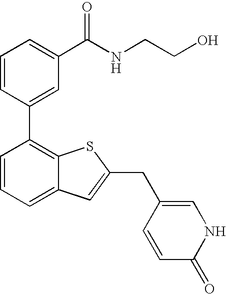

Working Example 289

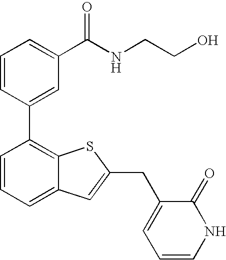

Working Example 290

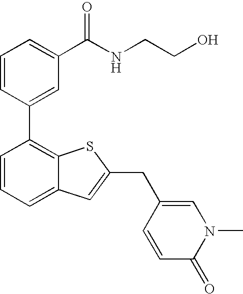

Working Example 291

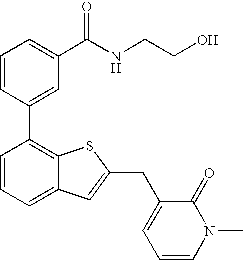

Working Example 292

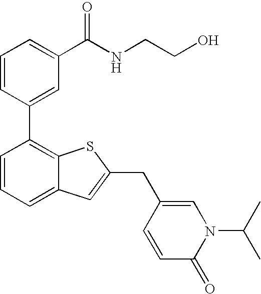

Working Example 293

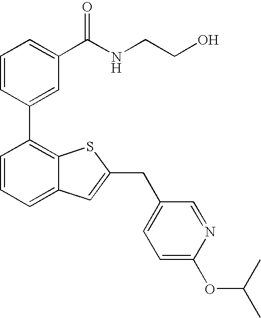

Working Example 294

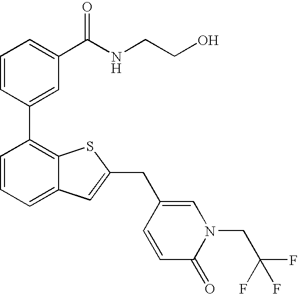

Working Example 295

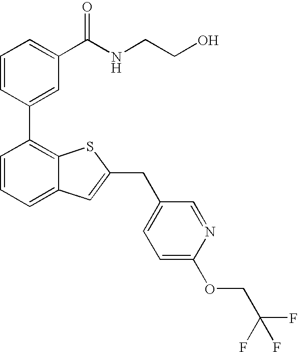

Working Example 296

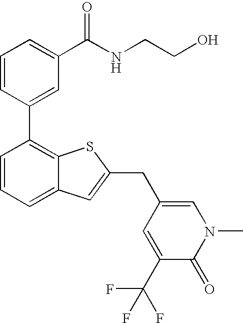

Working Example 297

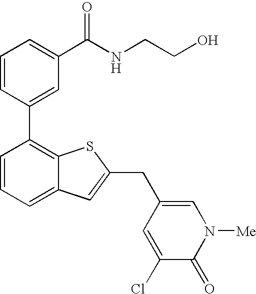

Working Example 298

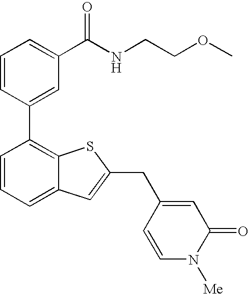

Working Example 299

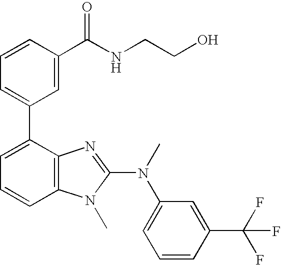

Working Example 300

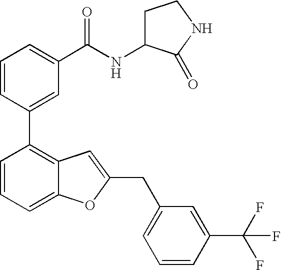

Working Example 301

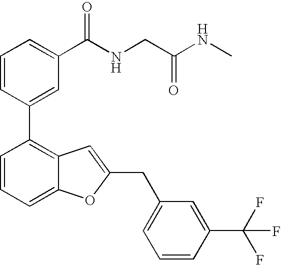

Working Example 302

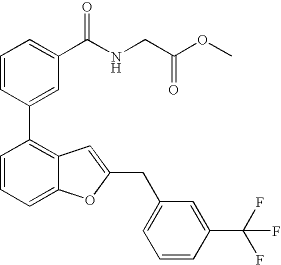

Working Example 303

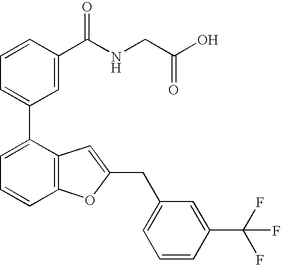

Working Example 304

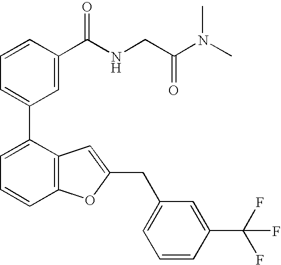

Working Example 305

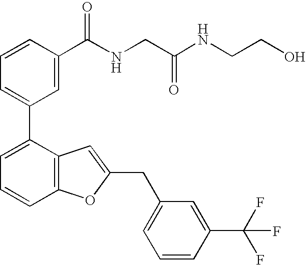

Working Example 306

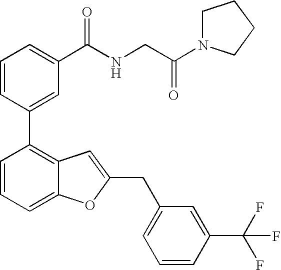

Working Example 307

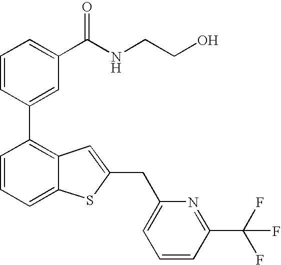

Working Example 308

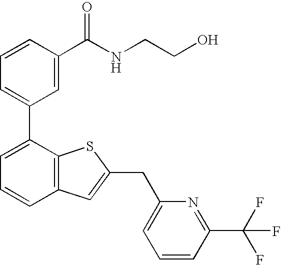

Working Example 309

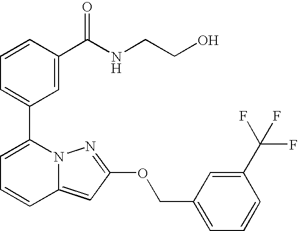

Working Example 310

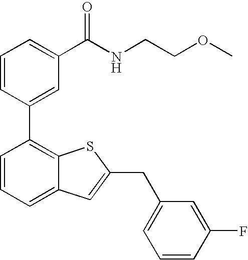

Working Example 311

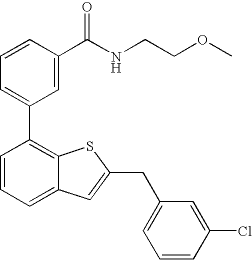

Working Example 312

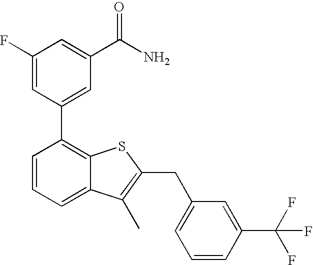

Working Example 313

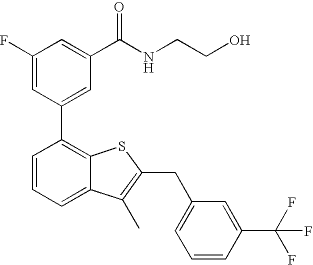

Working Example 314

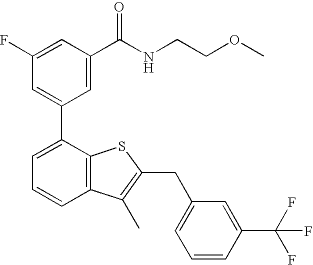

Working Example 315

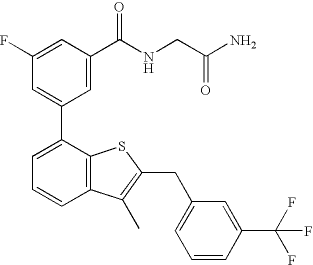

Working Example 316

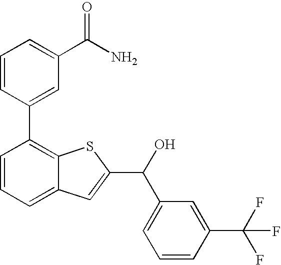

Working Example 317

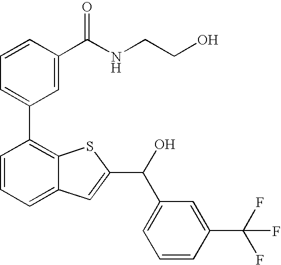

Working Example 318

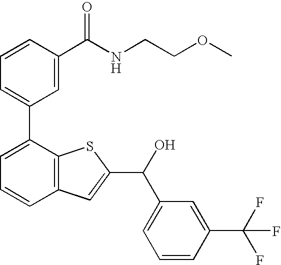

Working Example 319

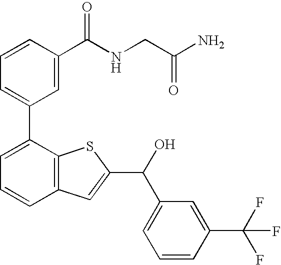

Working Example 320

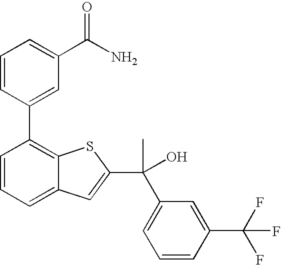

Working Example 321

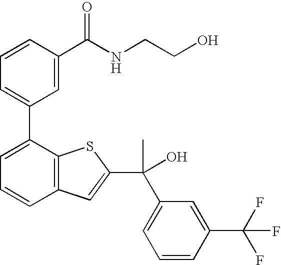

Working Example 322

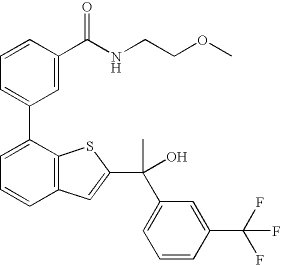

Working Example 323

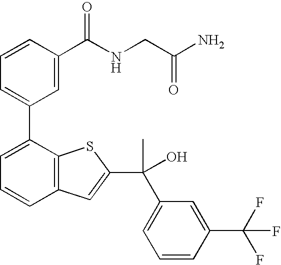

Working Example 324

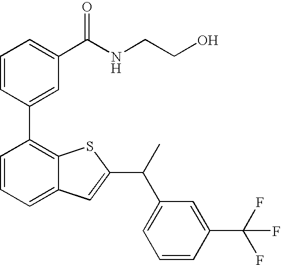

Working Example 325

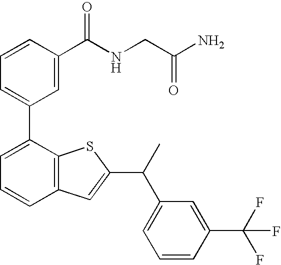

Working Example 326

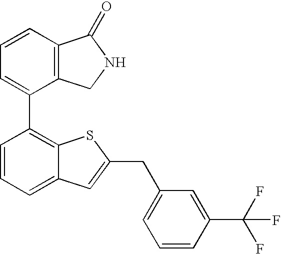

Working Example 327

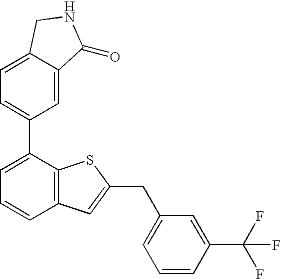

Working Example 328

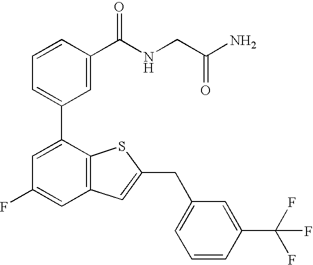

Working Example 329

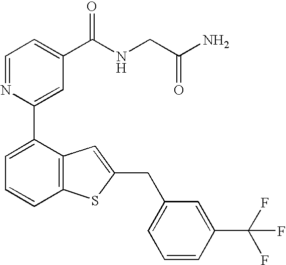

Working Example 330

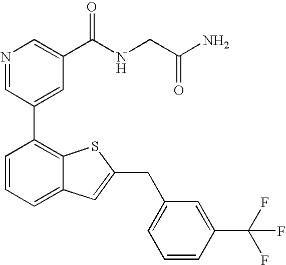

Working Example 331

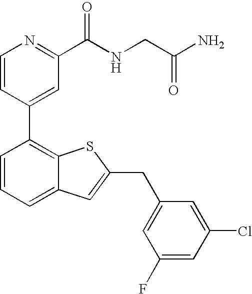

Working Example 332

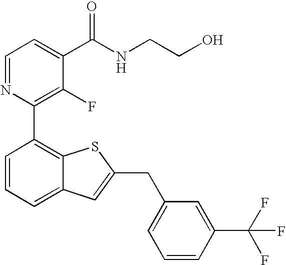

Working Example 333

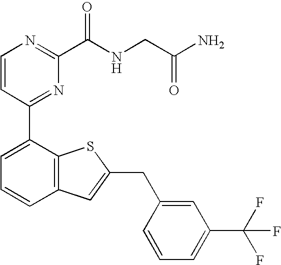

Working Example 334

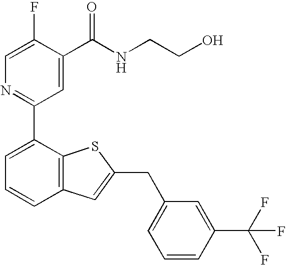

Working Example 335

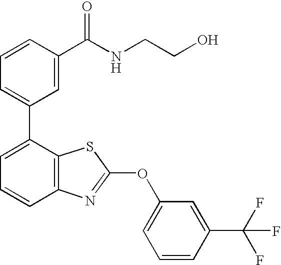

Working Example 336

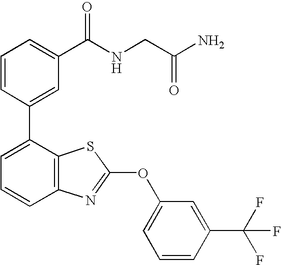

Working Example 337

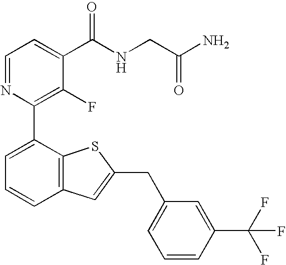

Working Example 338

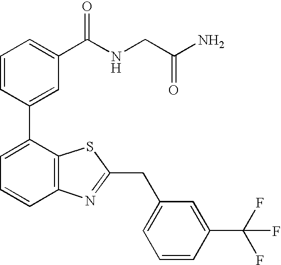

Working Example 339

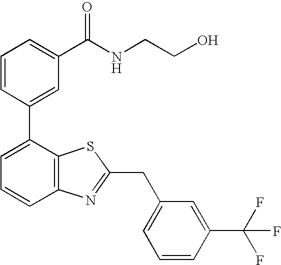

Working Example 340

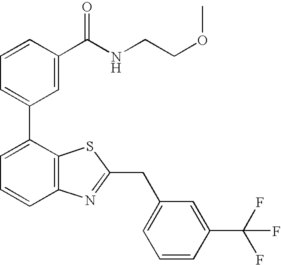

Working Example 341

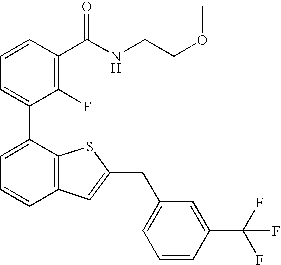

Working Example 342

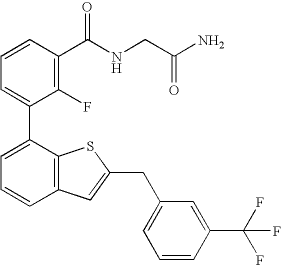

Working Example 343

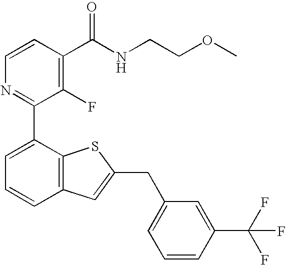

Working Example 344

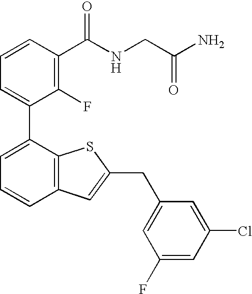

Working Example 345

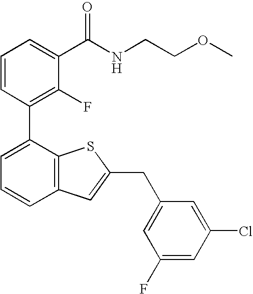

Working Example 346

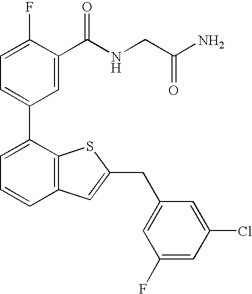

Working Example 347

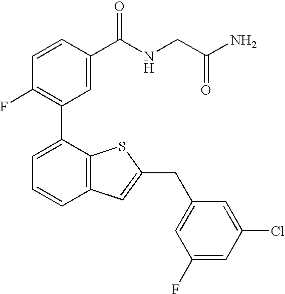

Working Example 348

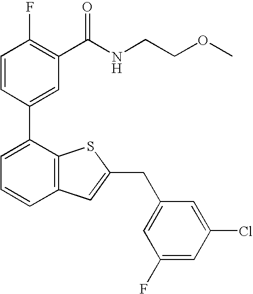

Working Example 349

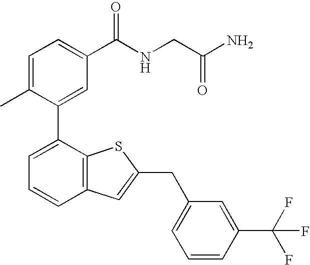

Working Example 350

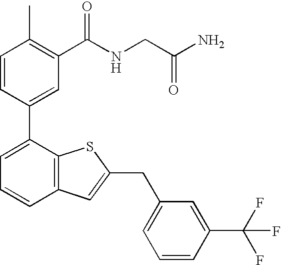

Working Example 351

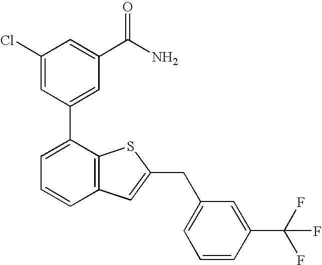

Working Example 352

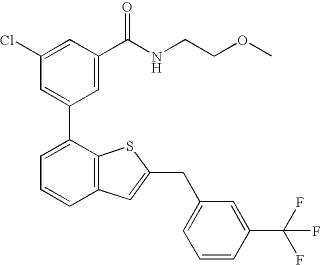

Working Example 353

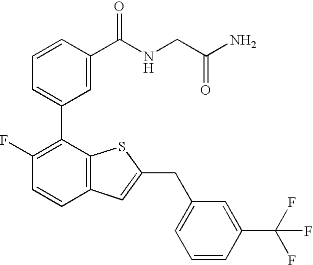

Working Example 354

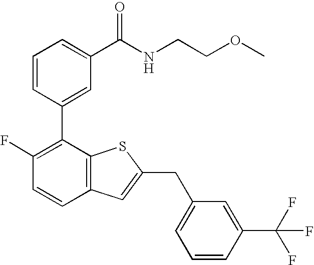

Working Example 355

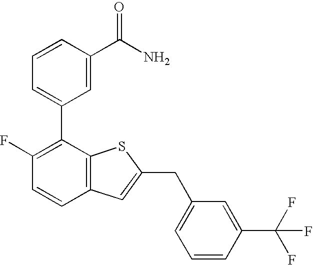

Working Example 356

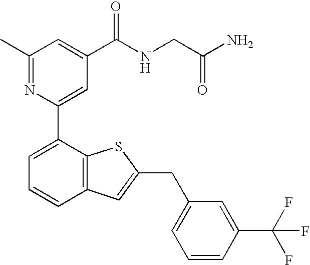

Working Example 357

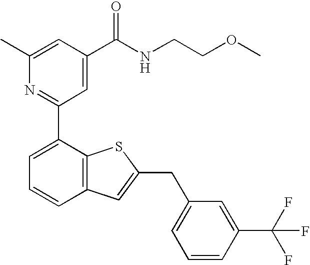

Working Example 358

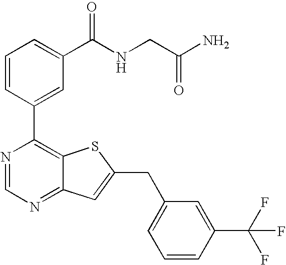

Working Example 359

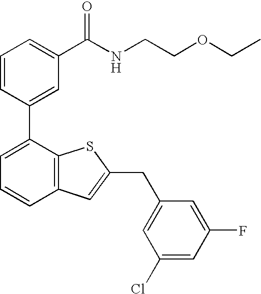

Working Example 360

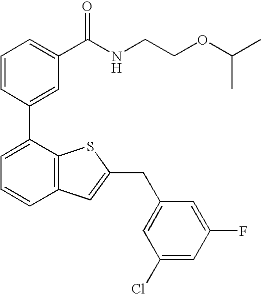

Working Example 361

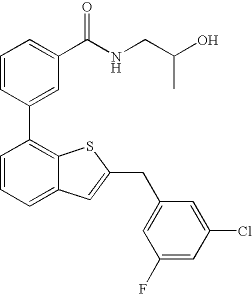

Working Example 362

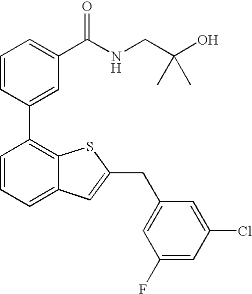

Working Example 363

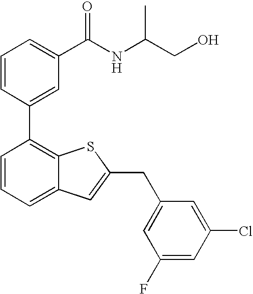

Working Example 364

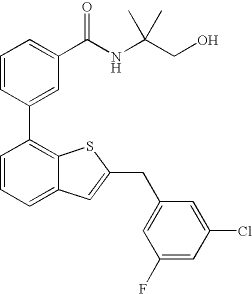

Working Example 365

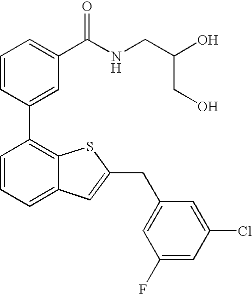

Working Example 366

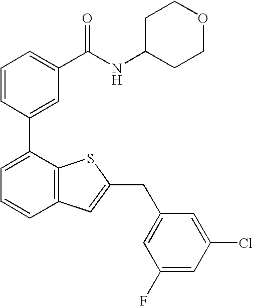

Working Example 367

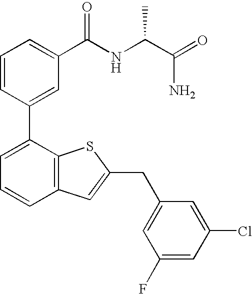

Working Example 368

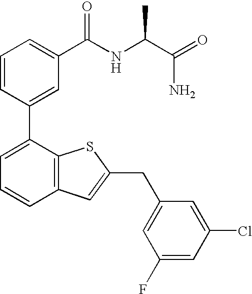

Working Example 369

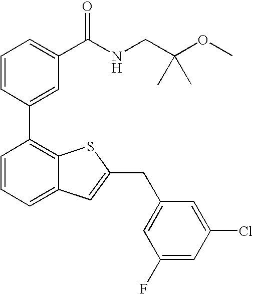

Working Example 370

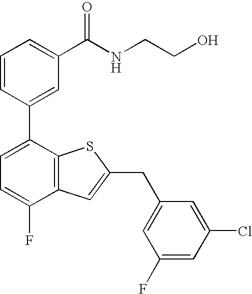

Working Example 371

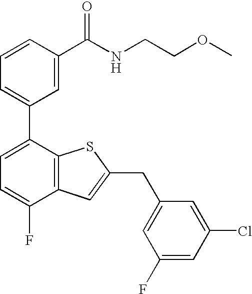

Working Example 372

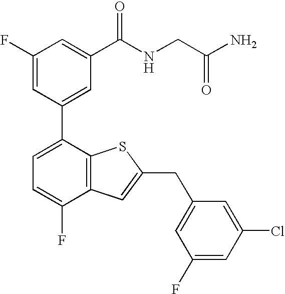

Working Example 373

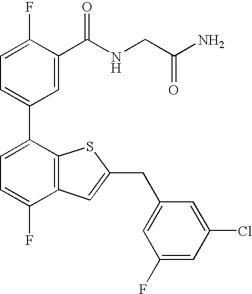

Working Example 374

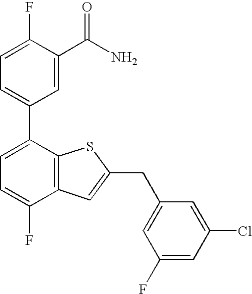

Working Example 375

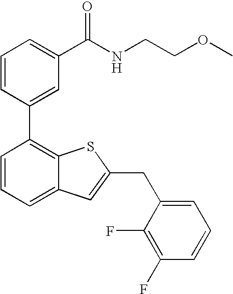

Working Example 376

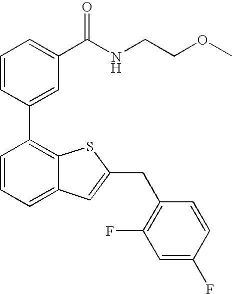

Working Example 377

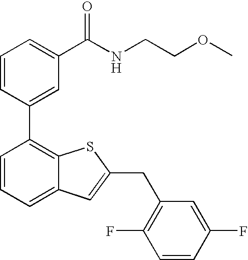

Working Example 378

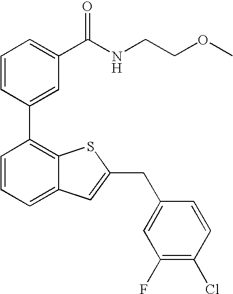

Working Example 379

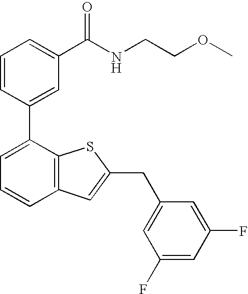

Working Example 380

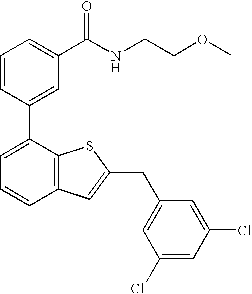

Working Example 381

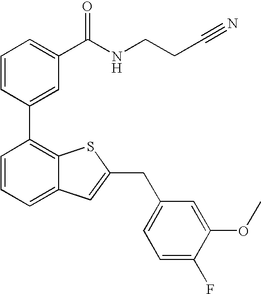

Working Example 382

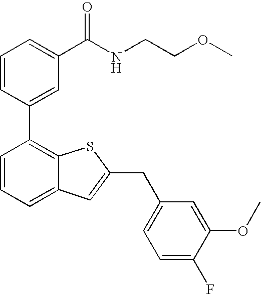

Working Example 383

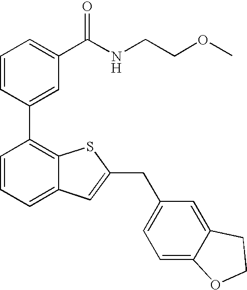

Working Example 384

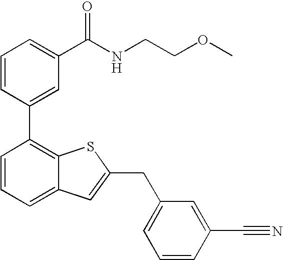

Working Example 385

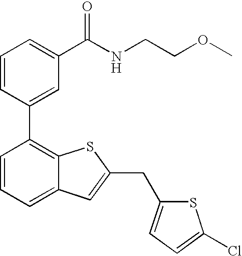

Working Example 386

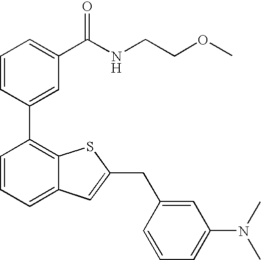

Working Example 387

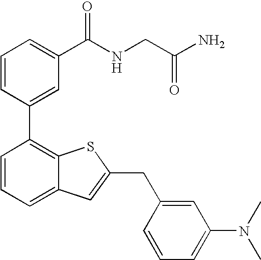

Working Example 388

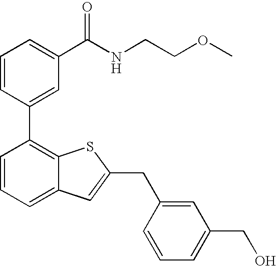

Working Example 389

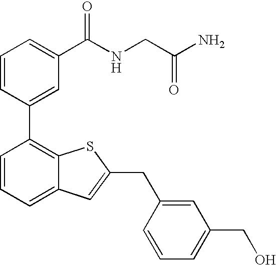

Working Example 390

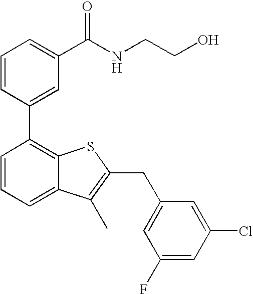

Working Example 391

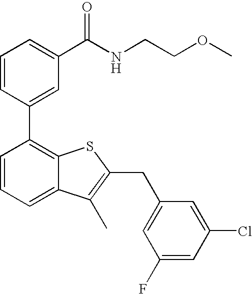

Working Example 392

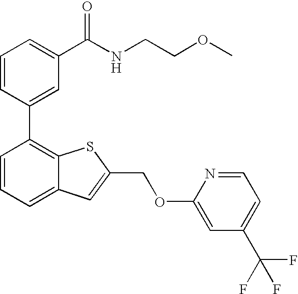

Working Example 393

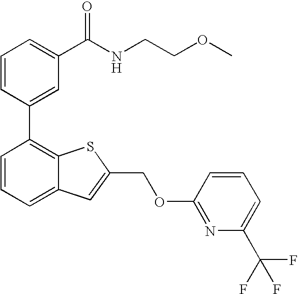

Working Example 394

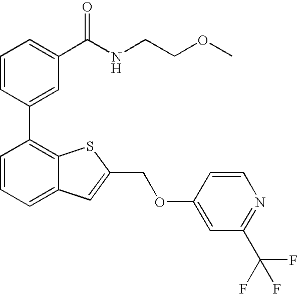

Working Example 395

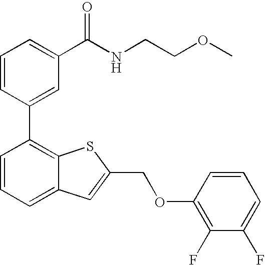

Working Example 396

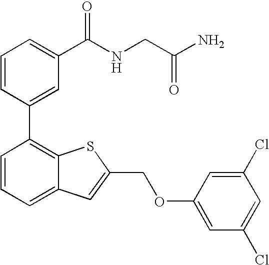

Working Example 397

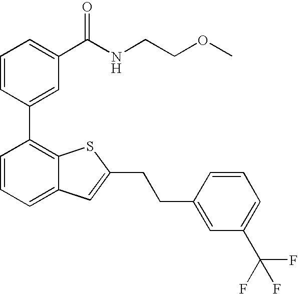

Working Example 398

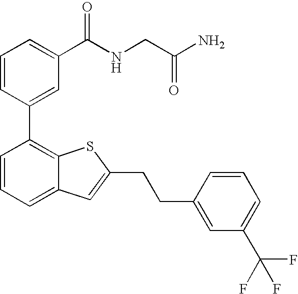

Working Example 399

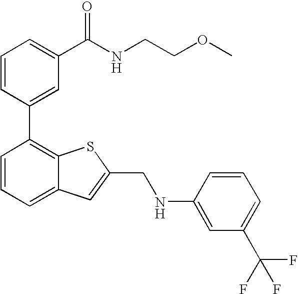

Working Example 400

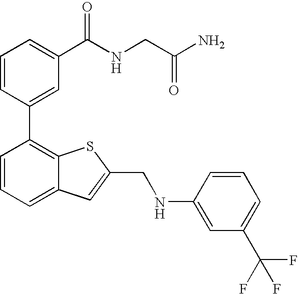

Working Example 401

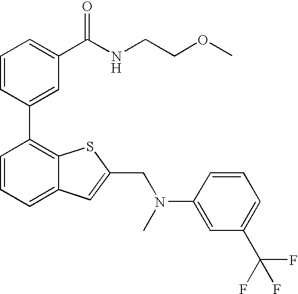

Working Example 402

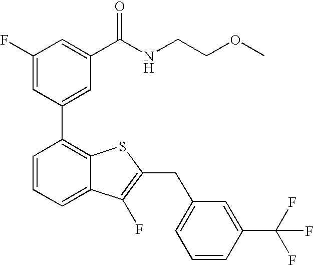

Working Example 403

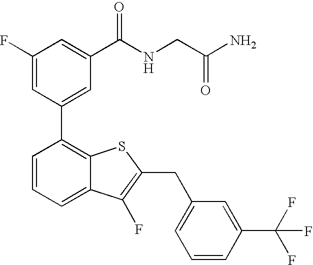

Working Example 404

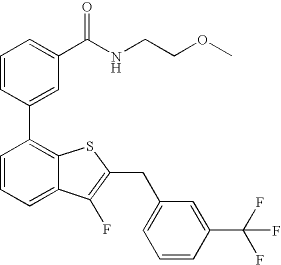

Working Example 405

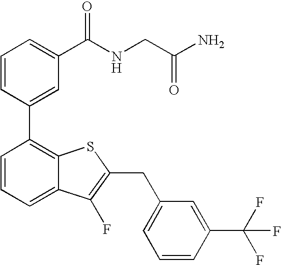

Working Example 406

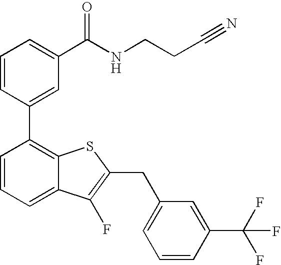

Working Example 407

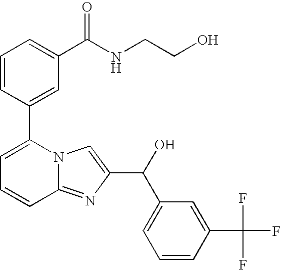

Working Example 408

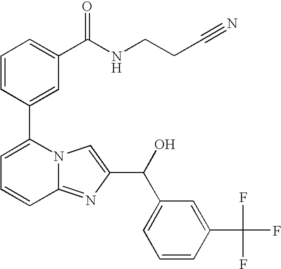

Working Example 409

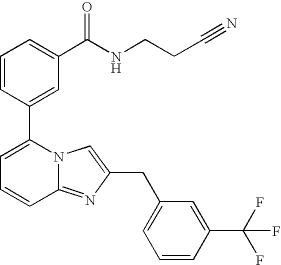

Working Example 410

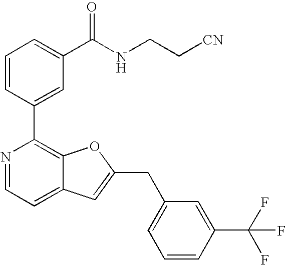

Working Example 411

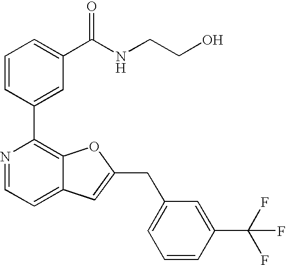

Working Example 412

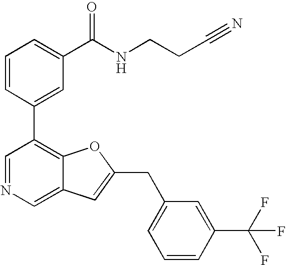

Working Example 413

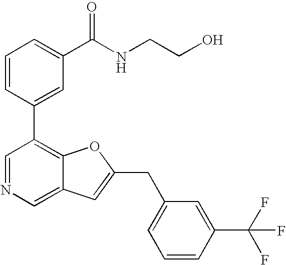

Working Example 414

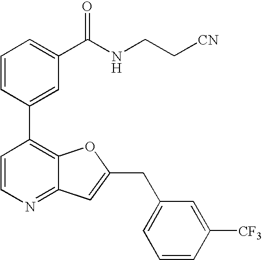

Working Example 415

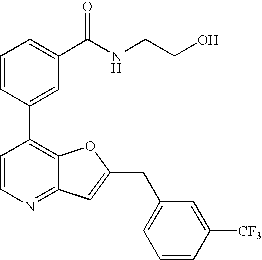

Working Example 416

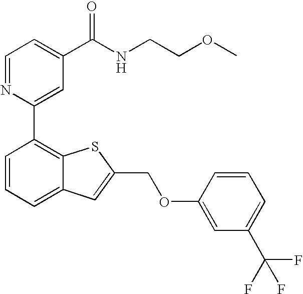

Working Example 417

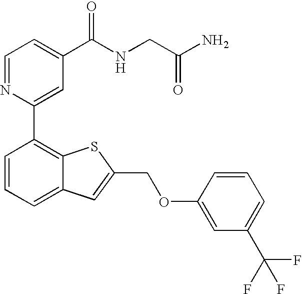

Working Example 418

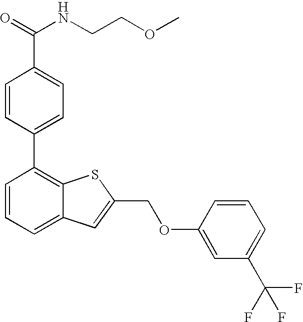

Working Example 419

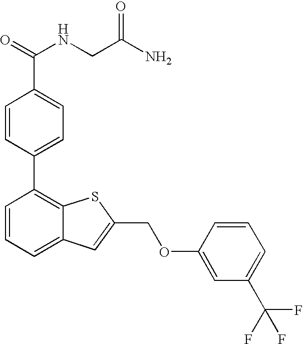

Working Example 420

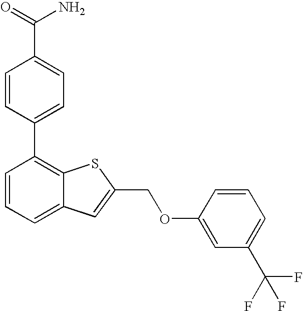

Working Example 421

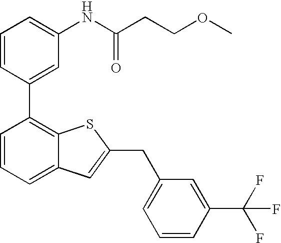

Working Example 422

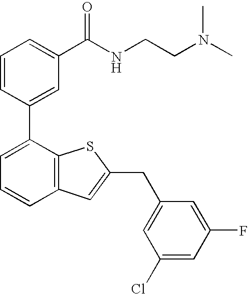

Working Example 423

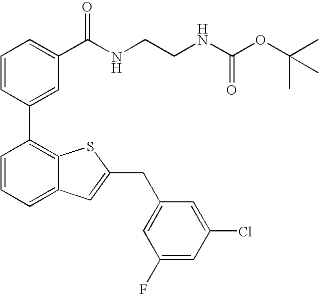

Working Example 424

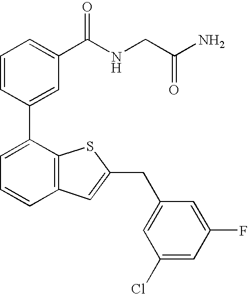

### Working Example 425-471

The compounds of Working Examples 425 through 471 were synthesized by reactions between various amines and 3-[2-[3-(trifluoromethyl)benzyl]-1-benzothiophene-7-yl]benzoic acid obtained in Reference Example 200 in the same manner as in Working Example 3. The synthesized compounds are shown in Table 5.

TABLE 5

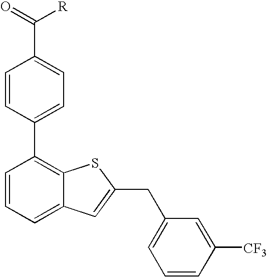

| Working | | |
|---|---|---|
| Example | | |
| No. | R | LCMS |
| 425 | 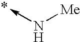 | 426 |
| 426 | 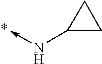 | 452 |
| 427 | 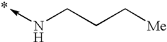 | 468 |
| 428 | 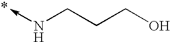 | 470 |
| 429 | 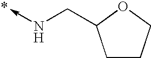 | 496 |
| 430 | 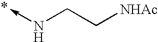 | 497 |
| 431 | 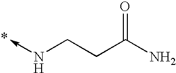 | 483 |
| 432 | 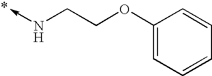 | 532 |
| 433 | 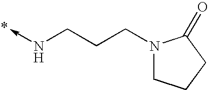 | 537 |
| 434 | 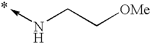 | 470 |
| 435 | 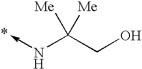 | 484 |
| 436 | 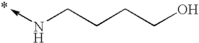 | 484 |
| 437 | 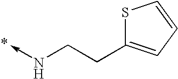 | 522 |
| 438 | 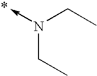 | 468 |
| 439 | 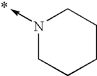 | 480 |
| 440 | 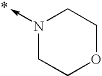 | 482 |
| 441 | 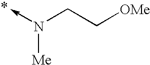 | 484 |
| 442 | 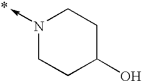 | 496 |
| 443 | 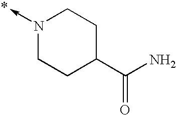 | 523 |
| 444 | 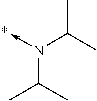 | 496 |
| 445 | 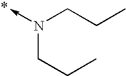 | 496 |
| 446 | 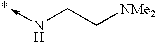 | 483 |
| 447 | 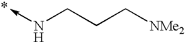 | 497 |
| 448 | 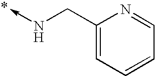 | 503 |
| 449 | 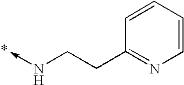 | 517 |
| 450 | 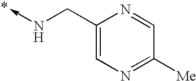 | 518 |
| 451 | 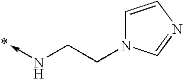 | 520 |
| 452 | 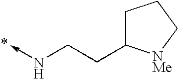 | 523 |
| 453 | 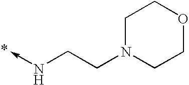 | 525 |
| 454 | 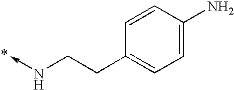 | 531 |
| 455 | 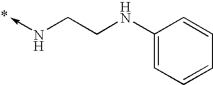 | 531 |
| 456 | 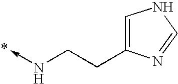 | 506 |
| 457 | 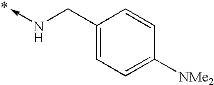 | 545 |
| 458 | 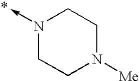 | 495 |
| 459 | 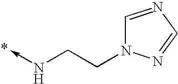 | 507 |
| 460 | 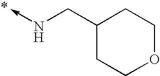 | 510 |
| 461 | 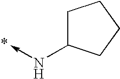 | 480 |
| 462 | 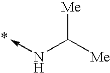 | 454 |
| 463 | 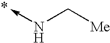 | 440 |
| 464 | 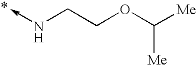 | 498 |
| 465 | 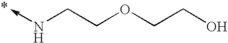 | 500 |
| 466 | 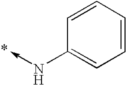 | 488 |
| 467 | 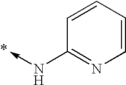 | 489 |
| 468 | 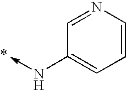 | 489 |
| 469 | 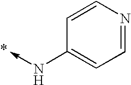 | 489 |
| 470 | 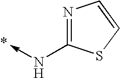 | 495 |
| 471 | 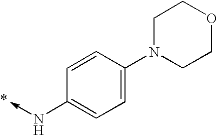 | 573 |

### Working Example 472-515

The compounds of Working Examples 472 through 515 were synthesized by reactions between various phenols and 3-[2-(hydroxymethyl)-1-benzothiophen-7-yl]-N-(2-methoxyethyl)benzamide obtained in Reference Example 178 in the same manner as in Reference Example 422. The synthesized compounds are shown in Table 6.

TABLE 6

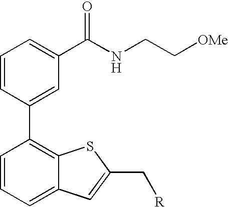

| Working | | |
|---|---|---|
| Example | | |
| No. | R | LCMS |
| 472 | 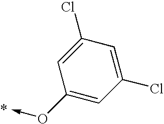 | 486 |
| 473 | 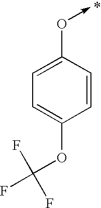 | 502 |
| 474 | 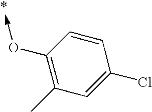 | 466 |
| 475 | 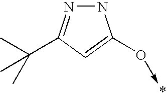 | 464 |
| 476 | 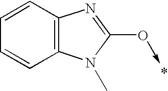 | 472 |
| 477 | 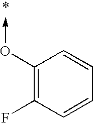 | 436 |
| 478 | 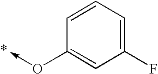 | 436 |
| 479 | 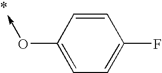 | 436 |
| 480 | 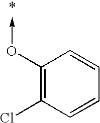 | 452 |
| 481 | 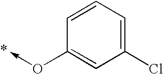 | 452 |
| 482 | 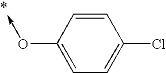 | 452 |
| 483 | 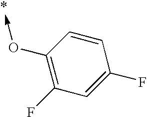 | 454 |
| 484 | 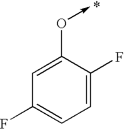 | 454 |
| 485 | 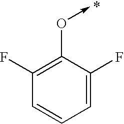 | 454 |
| 486 | 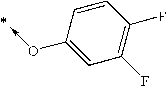 | 454 |
| 487 | 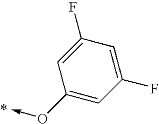 | 454 |
| 488 | 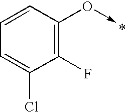 | 470 |
| 489 | 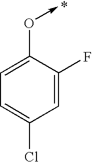 | 470 |
| 490 | 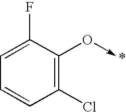 | 470 |
| 491 | 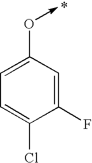 | 470 |
| 492 | 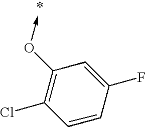 | 470 |
| 493 | 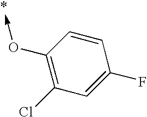 | 470 |
| 494 | 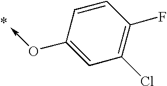 | 470 |
| 495 | 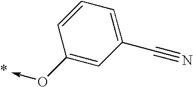 | 443 |
| 496 | 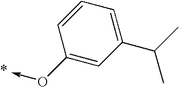 | 460 |
| 497 | 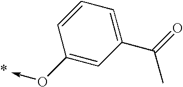 | 460 |
| 498 | 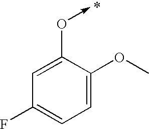 | 466 |
| 499 | 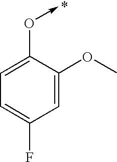 | 466 |
| 500 | 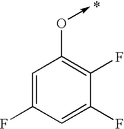 | 472 |
| 501 | 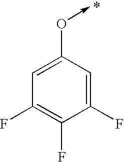 | 472 |
| 502 | 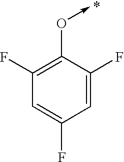 | 472 |
| 503 | 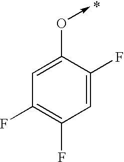 | 472 |
| 504 | 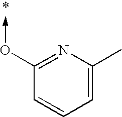 | 433 |
| 505 | 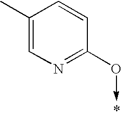 | 433 |
| 506 | 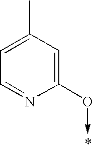 | 433 |
| 507 | 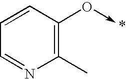 | 433 |
| 508 | 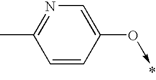 | 433 |
| 509 | 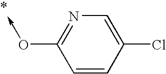 | 453 |
| 510 | 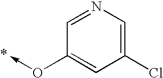 | 453 |
| 511 | 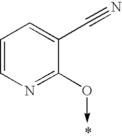 | 444 |
| 512 | 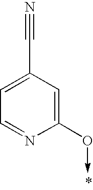 | 444 |
| 513 | 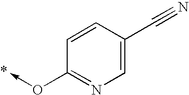 | 444 |
| 514 | 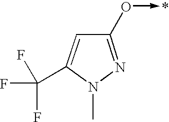 | 490 |
| 515 | 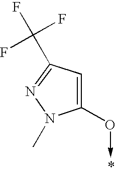 | 490 |

### Test Example 1

Increased intracellular cAMP levels in human GPR52 expression CHO cells 1×10⁴ human GPR52 expression CHO (dhfr-) cells were incubated with 1 μM test compound for 30 min at 37° C. in 30 μL assay buffer (HBSS (containing Ca²⁺ and Mg²⁺), 0.5% BSA, 100 μM IBMX, 100 μM Ro 20-1724, 5 mM HEPES (pH 7.55)) using an OptiPlate-384 (by PerkinElmer). The intracellular cAMP level was then assayed with an EnVision (by PerkinElmer) according to the protocol of the AlphaScreen cAMP Assay Kit (by PerkinElmer). The GPR52 agonist activity was calculated, assuming the intracellular cAMP level in the presence of 1 μM of 3-(6-(2-(3,4-dimethoxyphenyl)ethoxy)pyridine-2-yl)-N-(2-pyrrolidin-1-ylethyl)benzamide (Reference Example 136) to be 100% and assuming the intracellular cAMP level when DMSO was added instead of test compound to be 0%. The results are shown in Table 7.

TABLE 7

| Working | |
|---|---|
| Example No. | GPR52 agonist activity (%) |
| 7 | 82 |
| 21 | 74 |
| 23 | 75 |
| 65 | 75 |
| 88 | 59 |
| 99 | 67 |
| 143 | 80 |
| 147 | 88 |
| 159 | 80 |
| 165 | 108 |
| 174 | 90 |
| 179 | 89 |
| 184 | 82 |
| 191 | 94 |
| 198 | 88 |
| 221 | 81 |
| 222 | 62 |
| 230 | 85 |
| 252 | 80 |
| 265 | 93 |
| 271 | 94 |
| 292 | 61 |
| 326 | 92 |
| 360 | 79 |
| 405 | 113 |
| 413 | 83 |

### Test Example 2

Inhibition of Methamphetamine-Induced Locomotor Hyperactivity

Compounds of the present invention can be evaluated by assessing the inhibition of methamphetamine-induced locomotor hyperactivity in mice. The method of Okuyama et al was modified to test the inhibition of methamphetamine-induced locomotor hyperactivity (Life Science, Vol. 65, pages 2109-2125 (1999)).

Test Method

Locomotor activity was measured using a 32-channel locomotor activity measurement device MDC-LT (by BrainScience Idea Co., Ltd.), which emits infrared beams from the top of a cage (30×40×20 cm). 6- to 8-week old male ICR mice were acclimated for at least 45 min in the measurement cages, and then either test compound (30 mg/kg) dissolved in vehicle (0.5% methylcellulose aqueous solution) or the vehicle alone were orally administered. This oral administration was followed 60 min later by subcutaneous administration of methamphetamine (2 mg/kg).

Calculation of Activity

90 min after administration of methamphetamine, the number of times the laboratory animals passed through the infrared beams emitted inside the measurement cages was measured to quantify locomotor activity. The following equation was used to calculate the rate at which methamphetamine-induced locomotor hyperactivity was inhibited (activity inhibition rate (%)).

Activity inhibition rate (%)={1−(locomotor activity after treatment with methamphetamine in test compound treatment group÷locomotor activity after treatment with methamphetamine in vehicle treatment group)}×100

The results are shown in Table 8

TABLE 8

| Working Example No. | Activity Inhibition Rate (%) |
|---|---|
| 7 | 89 |
| 88 | 37 |
| 191 | 65 |
| 198 | 45 |
| 230 | 58 |
| 360 | 41 |

Preparation Example 1

| | |
|---|---|
| (1) Compound of Working Example 1 | 10.0 g |
| (2) Lactose | 70.0 g |
| (3) Corn starch | 50.0 g |
| (4) Soluble starch | 7.0 g |
| (5) Magnesium stearate | 3.0 g |

10.0 g of the compound of Working Example 1 and 3.0 g of magnesium stearate are granulated in a 70 mL aqueous solution of soluble starch (7.0 g as soluble starch), and the granules are then dried and mixed with 70.0 g of lactose and 50.0 g of corn starch (the lactose, corn starch, soluble starch, and magnesium stearate are all compliant with the Japanese Pharmacopoeia, Fourteenth Edition). The mixture is compressed to obtain tablets.

### INDUSTRIAL APPLICABILITY

The medicament of the present invention can be used as medicine for the prevention or treatment of mental illness such as schizophrenia.

1. A compound represented by formula (Ia): 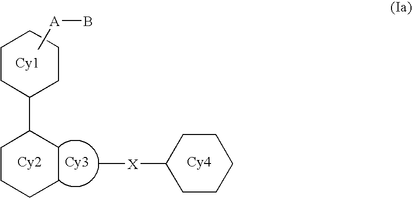
wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents; or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
a halogen atom,
a cyano group,
a hydroxy group,
a hydrocarbon-oxy group which may have one or more substituents,
a chain hydrocarbon group which may have one or more substituents (except for a methyl group substituted with a five-membered heterocyclic group),
a heterocyclic group which may have one or more substituents,
an amino group which may have one or more substituents,
an acyl group, and
a carboxy group which may be esterified;
ring Cy3 represents a five- or six-membered ring which may have one or more substituents;
X represents a C₁₋₂ alkylene which may be substituted with hydroxy, —Y—, Y—CH₂—, or —CH₂—Y—;
Y represents —O—, —NR^{b}—, or —S(O)ₘ—;
R^{b} represents a hydrogen atom or a substituent;
m represents an integer of 0 to 2; and
ring Cy4 represents a six-membered aromatic ring which may have one or more substituents (except for a sulfamoyl group which may have one or more substituents;
with the proviso that
a compound represented by the following formula: 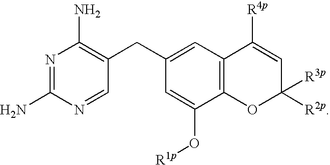
wherein
R^{1p} represents alkyl or cycloalkylalkyl;
R^{2p} and R^{3p} each independently represent an alkyl or a cycloalkyl or represent, together with an adjacent carbon atom, any of saturated three- to six-membered carbon rings or heterocyclic rings (where alkyl, cycloalkyl, a carbon ring, or a heterocyclic ring is unsaturated or saturated), and
R^{4p} represents aryl which may be substituted or heteroaryl which may be substituted,
a compound represented-by the following formula: 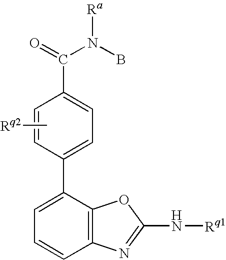
wherein
R^{q1} represents phenyl which may have one or more substituents,
R^{q2} represents hydrogen, or a substituent,
the other symbols are synonymous with those described above,
a compound represented by the following formula: 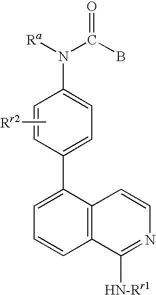
wherein
R^{r1} represents phenyl which may have one or more substituents,
R^{q2} represents hydrogen, or a substituent,
the other symbols are synonymous with those described above,
7-[4-(acetylamino)phenyl]-2-(benzylsulfanyl)-5-methyl-N-pheny-3,7-dihydro[1,2,4]triazolo[1,5-a]pyrimidine-6- carboxamide,
7-[4-(acetylamino)phenyl]-2-[(4-chlorobenzyl)sulfanyl]-N-(2,4-dimethylphenyl)-5-methyl-3,7-dihydro[1,2,4]triazolo[1,5-a]pyrimidine-6-carboxamide,
7-[4-(acetylamino)phenyl]-2-(benzylsulfanyl)-N-(2-methoxyphenyl)-5-methyl-3,7-dihydro[1,2,4]triazolo[1,5-a]pyrimidine-6-carboxamide,
7-[4-(acetylamino)phenyl]-2-[(2,4-dimethylbenzyl) sulfanyl]-N-(4-methoxyphenyl)-5-methyl-3,7-dihydro[1,2,4]triazolo[1,5-a]pyrimidine-6-carboxamide,
7-[4-(acetylamino)phenyl]-2-(benzylsulfanyl)-N-(2,4-dimethylphenyl)-5-methyl-3,7-dihydro[1,2,4]triazolo [1,5-a]pyrimidine-6-carboxamide,
N-(3-(2-((4-chloro-2-methoxy-6-methylphenyl)amino)-1-methyl-1H-benzoimidazol-7-yl)phenyl)acetamide),
tert-butyl methyl[4-[2-methyl-1-(phenylsulfonyl)-1H-pyrrolo[2,3-b]pyridin-4-yl]phenyl]carbamate,
tert-butyl (4-[3-[(4-methoxybenzyl)amino]imidazo[1,5-a]pyridin-5-yl]phenyl)carbamate,
tert-butyl 1-[4-(diethylcarbamoyl)phenyl]-6-methoxy-7-phenoxy-3,4-dihydroisoquinoline-2(1H)-carboxylate,
tert-butyl 1[4-(diethylcarbamoyl)phenyl]-7-(4-fluorophenoxy)-6-methoxy-3,4-dihydroisoquinoline-2(1H)-carboxylate,
tert-butyl 1-[4-(diethylcarbamoyl)phenyl]-6-methoxy-7-(4-methoxyphenoxy)-3,4-dihydroisoquinoline-2(1H)-carboxylate,
tert-butyl 1-[4-(diethylcarbamoyl)phenyl]-6-methoxy-7-(pyridin-3-yloxy)-3,4-dihydroisoquinoline-2(1H)-carboxylate,
1-[4-(1-benzyl-1H-pyrazolo[3,4-c]pyridin-4-yl)phenyl]-3-[3-(trifluoromethyl)phenyl]urea,
1-[4-[1-(4-methoxybenzyl)-1H-pyrazolo[3,4-c]pyridin-4-yl]phenyl]-3-[3-(trifluoromethyl)phenyl]urea,
3-chloro-2-[6-[(2-chloro-4-fluorophenyl)sulfanyl]-2-oxo-3,4-dihydropyrido[3,2-d]pyrimidin-1(2H)-yl]benzamide,
3,5-dichloro-4-[6-[(2,4-difluorophenyl)sulfanyl]-2-oxo-3,4-dihydropyrido[3,2-d]pyrimidin-1(2H)-yl]benzamide,
3,5-dichloro-4-[6-[(2,4-difluorophenyl)sulfanyl]-2-oxo-3,4-dihydropyrido[3,2-d]pyrimidin-1(2H)-yl]-N-[2-(dimethylamino)ethyl]benzamide,
2-chloro-N-(3-chloro-4-[2-[(4-fluorophenyl)sulfanyl]-6-oxo-7,8-dihydro-6H-pyrimido[1,6-b]pyridazin-5-yl]phenyl)acetamide,
N-(3-chloro-4-[2-[(4-fluorophenyl)sulfanyl]-6-oxo-7,8-dihydro-6H-pyrimido[1,6-b]pyridazin-5-yl]phenyl)acetamide,
N-(3-chloro-4-[2-[(4-fluorophenyl)sulfanyl]-6-oxo-7,8-dihydro-6H-pyrimido[1,6-b]pyridazin-5-yl]phenyl)-2-morpholin-4-ylacetamide,
N-(4-[2-[(3,4,5-trimethoxyphenyl)amino]-1,3-benzoxazol-7-yl]phenyl)acetamide,
N-(3-[2-[(3,4,5-trimethoxyphenyl)amino]-1,3-benzoxazol-7-yl]phenyl)acetamide,
N-(2-amino-4-[2-[(3,4,5-trimethoxyphenyl)amino]-1,3-benzoxazol-7-yl]phenyl)formamide,
7-[4-(acetylamino)phenyl]-2-[(2,4-dimethylbenzyl)sulfanyl]-5-methyl-N-phenyl-3,7-dihydro[1,2,4]triazolo[1,5-a]pyrimidine-6-carboxamide,
5-[(3S)-3-(dibenzylamino)-3,4-dihydro-2H-chromen-5-yl]-2-methoxy-N,N-dimethylpyridine-3-carboxamide,
5-[(3S)-3-(dibenzylamino)-3,4-dihydro-2H-chromen-5-yl]-2-methoxy-N-methylpyridine-3-carboxamide,
5-[(3S)-3-(dibenzylamino)-3,4-dihydro-2H-chromen-5-yl]-2-methoxy-N-methylpyridine-3-carboxamide, and
N-(6-[1-[(4-methylphenyl)sulfonyl]-1H-pyrrolo[2,3-b]pyridin-4-yl]pyridin-2-yl)acetamide, are excluded;
or a salt thereof.

2. A compound represented by formula (I): 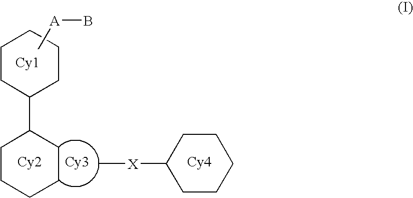
wherein
A represents —CONR^{a}— or —NR^{a}CO—,
R^{a} represents a hydrogen atom or a substituent,
B represents a hydrogen atom or a substituent,
or alternatively, when A is —CONR^{a}—, R^{a} and B may form together with an adjacent nitrogen atom a nitrogen-containing heterocyclic group which may have one or more substituents;
ring Cy1 represents a six-membered aromatic ring which may have one or more substituents in addition to a group represented by -A-B;
ring Cy2 represents a six-membered ring which may have one or more substituents selected from
a halogen atom,
a cyano group,
a hydroxy group,
a hydrocarbon-oxy group which may have one or more substituents,
a chain hydrocarbon group which may have one or more substituents (except for a methyl group substituted with a five-membered heterocyclic group),
a heterocyclic group which may have one or more substituents,
an amino group which may have one or more substituents,
an acyl group, and
a carboxy group which may be esterified;
ring Cy3 represents a five- or six-membered ring which may have one or more substituents;
X represents a C₁₋₂ alkylene, —Y—, Y—CH₂—, or —CH₂—Y—;
Y represents —O—, —NR^{b}—, or —S(O)ₘ—;
R^{b} represents a hydrogen atom or a substituent;
m represents an integer of 0 to 2; and
ring Cy4 represents a six-membered aromatic ring which may have one or more substituents (except for a sulfamoyl group which may have one or more substituents;
with the proviso that
a compound represented by the following formula: 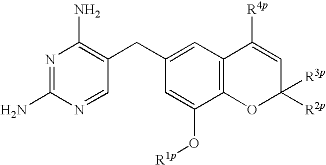
wherein
R^{1p} represents alkyl or cycloalkylalkyl;
R^{2p} and R^{3p} each independently represent an alkyl or a cycloalkyl or represent, together with an adjacent carbon atom, any of saturated three- to six-membered carbon rings or heterocyclic rings (where alkyl, cycloalkyl, a carbon ring, or a heterocyclic ring is unsaturated or saturated), and
R^{4p} represents aryl which may be substituted or heteroaryl which may be substituted,
7-[4-(acetylamino)phenyl]-2-(benzylsulfanyl)-5-methyl-N-phenyl-3,7-dihydro[1,2,4]triazolo[1,5-a]pyrimidine-6-carboxamide,
7-[4-(acetylamino)phenyl]-2-[(4-chlorobenzyl)sulfanyl]-N-(2,4-dimethylphenyl)-5-methyl-3,7-dihydro[1,2,4]triazolo[1,5-a]pyrimidine-6-carboxamide,
7-[4-(acetylamino)phenyl]-2-(benzylsulfanyl)-N-(2-methoxyphenyl)-5-methyl-3,7-dihydro[1,2,4]triazolo[ 1 ,5-a]pyrimidine-6-carboxamide,
7-[4-(acetylamino)phenyl]-2-[(2,4-dimethylbenzyl) sulfanyl]-N-(4-methoxyphenyl)-5-methyl-3,7-dihydro[1,2,4]triazolo[1,5-a]pyrimidine-6-carboxamide,
7-[4-(acetylamino)phenyl]-2-(benzylsulfanyl)-N-(2,4-dimethylphenyl)-5-methyl-3,7-dihydro[1,2,4]triazolo[1,5-a]pyrimidine-6-carboxamide, and
N-(3-(2-((4-chloro-2-methoxy-6-methylphenyl)amino)-1-methyl-1H-benzoimidazol-7-yl)phenyl)acetamide) are excluded;
or a salt thereof.

3. The compound according to claim 2, wherein
ring Cy1 is a benzene ring or a pyridine ring.

4. The compound according to claim 2, wherein
ring Cy2 is a six-membered ring which may have one or more substituents selected from a halogen atom, an alkyl group which may have one or more substituents, and an alkoxy group which may have one or more substituents.

5. The compound according to claim 2, wherein
ring Cy3 is a five- or six-membered ring which may have one or more substituents selected from a halogen atom, an alkyl group which may have one or more substituents, and an alkoxy group which may have one or more substituents.

6. The compound according to claim 2, wherein
ring Cy4 is a benzene ring or a pyridine ring, which may have one or more substituents a substituent (except for a sulfamoyl group which may have one or more substituents).

7. The compound according to claim 2, wherein
the chemical formula (I) is as follows:

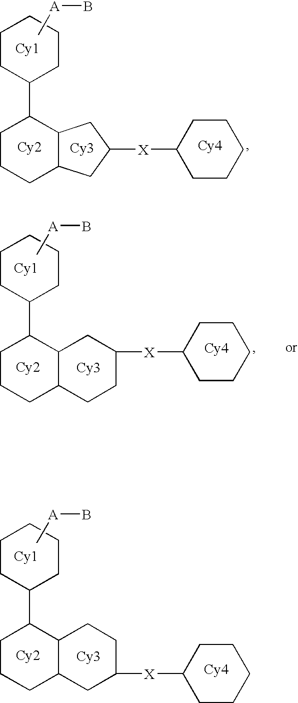

8. The compound according to claim 2, wherein
the chemical formula (I) is as follows: 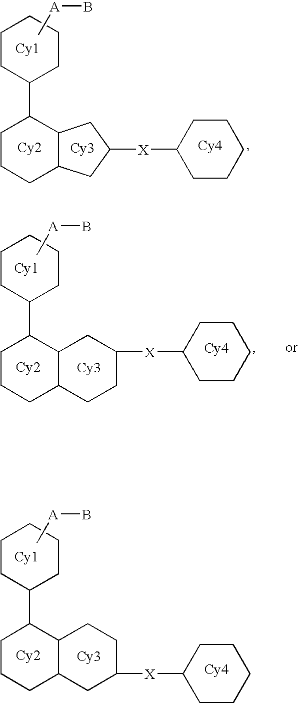
ring Cy1 is a benzene ring or a pyridine ring;
ring Cy2 is a six-membered ring which may have one or more substituents selected from a halogen atom, an alkyl group which may have one or more substituents, and an alkoxy group which may have one or more substituents;
ring Cy3 is a five- or six-membered ring which may have one or more substituents selected from a halogen atom, an alkyl group which may have one or more substituents, and an alkoxy group which may have one or more substituents; and
ring Cy 4 is a benzene ring or a pyridine ring, which may have one or more substituents.

9. The compound according to claim 1, wherein
the chemical formula (Ia) is as follows: 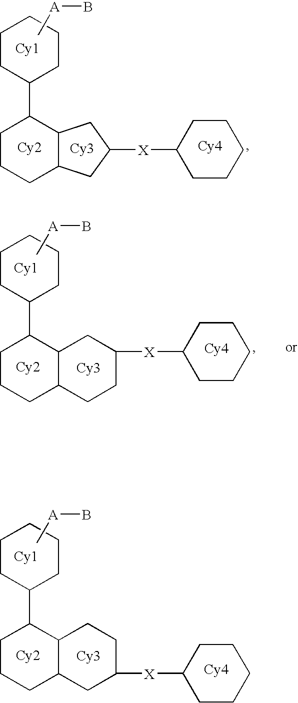
A represents —CONR^{a}— or —NR^{a}CO—;
R^{a} represents a hydrogen atom or a C₁₋₆ alkyl group a substituent;
B represents
1) a hydrogen atom,
2) a C₁₋₆ alkyl group which may have one or more substituents selected from
a) cyano group,
b) a hydroxy group,
c) a C₁₋₆ alkoxy group,
d) a C₆₋₁₄ aryloxy group,
e) a carbamoyl group, and
f) an amino group which may be substituted with one or two substitutes selected from a C₁₋₆ alkyl group, a C₆₋₁₄ aryl group, a C₁₋₆ alkylcarbonyl group,
g) a C₆₋₁₄ aryl group which may be substituted with an amino group which may be substituted with one or two C₁₋₆ alkyl group,
h) a five- or six-membered heterocyclic group which may be substituted with a substituent selected from a C₁₋₆ alkyl group and an oxo group,
i) a C₁₋₆ alkylsulfanyl group,
j) a C₁₋₆ alkylsulfinyl group, and
k) a C₁₋₆ alkylsulfonyl group,
3) a C₃₋₆ cycloalkyl group which may be substituted with a hydroxy-group,
4) a C₆₋₁₄ aryl group which may be substituted with a five- or six-membered heterocyclic group, or
5) a five- to ten-membered heterocyclic group which may be substituted with a halogen atom,
or alternatively, when A is —CONR^{a}—, R^{a} and B may form together with an adjacent nitrogen atom a nitrogen-containing six-membered heterocyclic group which may have one or more substituents selected from a hydroxy group, a C₁₋₆ alkyl group, and a carbamoyl group;
ring Cy1 represents a benzene ring or a pyridine ring, each of which may have one or more substituents selected from a halogen atom and a C₁₋₆ alkyl group;
ring Cy2 represents a benzene ring or a pyridine ring which may have one or more substituents selected from a halogen atom and a C₁₋₆ alkoxy group;
ring Cy3 represents a five- or six-membered heterocyclic ring which may have one or more substituents selected from
1) a C₁₋₆ alkyl group
2) an oxo group and
3) a halogen atom;
X represents —CH₂—, —CH₂—CH₂—, —CH(CH₃)—, —NH—, —CH(OH)—, —CH₂—O—, —C(CH₃)(OH)—, or —O—; and
ring Cy4 represents
1) a benzene ring which may have one or more substituents selected from
a) a halogen atom,
b) a C₁₋₆ alkyl group which may be halogenated or hydroxylated,
c) a C₁₋₆ alkoxy group
d) an amino group which may be substituted with one or two C₁₋₆ alkyl groups, and
e) a C₁₋₆ alkylsulfonyl group,
2) a pyridine ring which may have one or more substituents selected from
a) a C₁₋₆ alkyl group which may be halogenated, and
b) a C₁₋₆ alkoxy,
3) a pyridone ring which may have one or more substituents selected from
a) a halogen atom, and
b) a C₁₋₆ alkyl group which may be halogenated.

10. The compound according to claim 2, wherein
the chemical formula (I) is as follows: 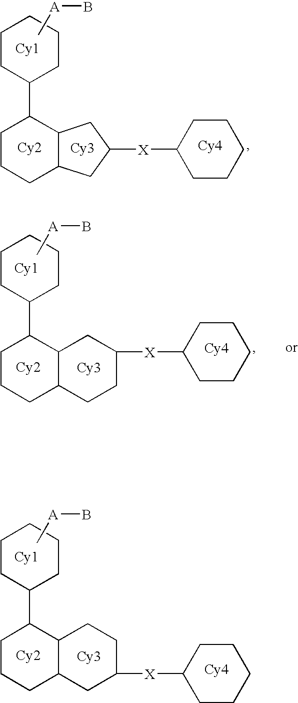
A represents —CONR^{a}— or —NR^{a}CO—;
R^{a} represents a hydrogen atom or a C₁₋₆ alkyl group a substituent;
B represents
1) a hydrogen atom,
2) a C₁₋₆ alkyl group which may have one or more substituents selected from
a) a cyano group,
b) a hydroxy group,
c) a C₁₋₆ alkoxy group,
d) a C₆₋₁₄ aryloxy group,
e) a carbamoyl group, and
f) an amino group which may be substituted with one or two substitutes selected from a C₁₋₆ alkyl group, a C₆₋₁₄ aryl group, a C₁₋₆ alkylcarbonyl group,
g) a C₆₋₁₄ aryl group which may be substituted with an amino group which may be substituted with one or two C₁₋₆ alkyl groups,
h) a five- or six-membered heterocyclic group which may be substituted with a substituent selected from a C₁₋₆ alkyl group and an oxo group,
i) a C₁₋₆ alkylsulfanyl group,
j) a C₁₋₆ alkylsulfinyl group, and
k) a C₁₋₆ alkylsulfonyl group,
3) a C₃₋₆ cycloalkyl group which may be substituted with a hydroxy group,
4) a C₆₋₁₄ aryl group which may be substituted with a five- or six-membered heterocyclic group, or
5) a five- to ten-membered heterocyclic group which may be substituted with a halogen atom,
or alternatively, when A is —CONR^{a}—, R^{a} and B may form together with an adjacent nitrogen atom a nitrogen-containing six-membered heterocyclic group which may have one or more substituents selected from a hydroxy group, a C₁₋₆ alkyl group, and a carbamoyl group;
ring Cy1 represents a benzene ring or a pyridine ring;
ring Cy2 represents a benzene ring or a pyridine ring which may have one or more substituents selected from a halogen atom and a C₁₋₆ alkoxy group;
ring Cy3 represents a five- or six-membered heterocyclic ring which may have one or more substituents selected from a C₁₋₆ alkyl group and an oxo group;
X represents a C₁₋₂ alkylene, —NH—, or —O—; and
ring Cy4 represents a benzene ring which may have one or more substituents selected from a halogen atom, a C₁₋₆ alkyl group which may be halogenated, and a C₁₋₆ alkoxy group.

11. The compound according to claim 9,
wherein
the chemical formula (Ia) is the chemical formula (II) 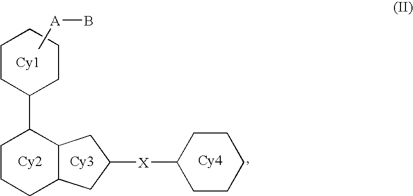
A is —CONR^{a},
R^{a} is a hydrogen atom, or a C₁₋₆ alkyl group,
B is
1) a hydrogen atom,
2) a C₁₋₆ alkyl group,
which may have one or more substituents selected from
a) a cyano group,
b) a hydroxy group,
c) a C₁₋₆ alkoxy group,
d) a carbamoyl group,
e) an amino group which may have one or two substituents selected from a C₁₋₆ alkyl group, a C₆₋₁₄ aryl group, and a C₁₋₆ alkyl-carbonyl group
f) a C₁₋₆ alkylsulfinyl group,
ring Cy1 is a benzene ring or a pyridine ring
ring Cy2 is a benzene ring or a pyridine ring which may have one or more substituents selected from a halogen atom and a C₁₋₆ alkoxy group,
ring Cy3 is a 5-membered heterocyclic ring which may have one or more substituents selected from C₁₋₆ alkyl group, and an oxo group
X is C₁₋₂ alkylene, or —O—,
ring Cy4 is
a benzene ring
which may have one or more substituents selected from
1) a halogen atom,
2) a C₁₋₆ alkyl group which may be halogenated,
3) a C₁₋₆ alkoxy group, and
4) a C₁₋₆ alkylsulfonyl group.

12. The compound according to claim 10, wherein the chemical formula (I) is the chemical formula(II) 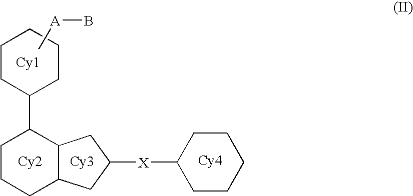
A is —CONR^{a},
R^{a} is a hydrogen atom, or a C₁₋₆ alkyl group,
B is
1) a hydrogen atom,
2) a C₁₋₆ alkyl group,
which may have one or more substituents selected from
a) a cyano group,
b) a hydroxy group,
c) a C₁₋₆ alkoxy group,
d) a carbamoyl group,
e) an amino group, which may have one or two substituents selected from a C₁₋₆ alkyl group, a C₆₋₁₄ aryl group, and a C₁₋₆ alkyl-carbonyl group
f) a C₁₋₆ alkylsulfinyl group,
ring Cy1 is a benzene ring, or a pyridine ring,
ring Cy2 is a benzene ring, or a pyridine ring which may have one or more substituents selected from
a halogen atom, and a C₁₋₆ alkoxy group,
ring Cy3 is a 5-membered heterocyclic ring which may have one or more substituents selected from
C₁₋₆ alkyl group, and an oxo group,
X is
C₁₋₂ alkylene, or —O—,
ring Cy4 is
a benzene ring
which may have one or more substituents selected from
1) a halogen atom,
2) a C₁₋₆ alkyl group which may be halogenated, and
3) a C₁₋₆ alkoxygroup,

13. The compound according to claim 12, wherein the skeleton of the moiety represented by 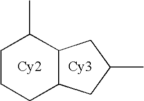
of the chemical formula (II) is a fused ring selected from

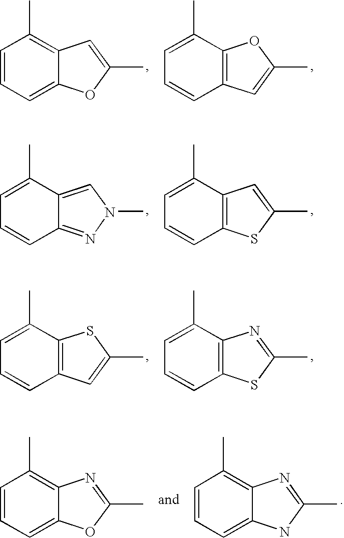

14. The compound according to claim 12, the skeleton of the moiety represented by 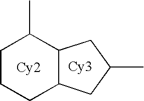
the chemical formula (II) is a fused ring selected from

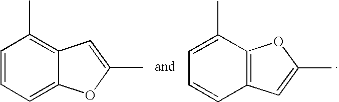

15. The compound according to claim 12, the skeleton of the moiety represented by 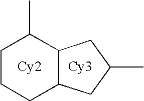
of the chemical formula (II) is a fused ring selected from

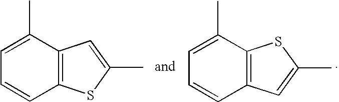

16. The compound according to claim 10, wherein the chemical formula (I) is the chemical formula (III) 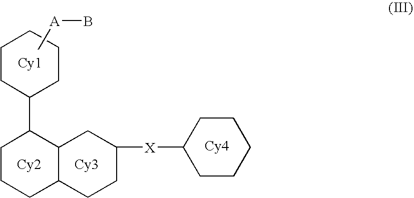
A is —CONR^{a},
R^{a} is a hydrogen atom,
B is
a C₁₋₆ alkyl group which may have one or more substituents selected from
a) a cyano group, and
b) a hydroxy group,
ring Cy1 is a benzene ring
the skeleton of the moiety represented by 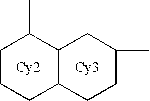
of the chemical formula (III) is a fused ring represented by 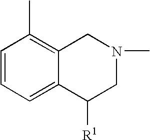
wherein R¹ is a hydrogen atom, or a C₁₋₆ alkyl group
X is C₁₋₂ alkylene,
ring Cy4 is a benzene ring which is substituted with
C₁₋₆ alkyl group which may be halogenated.

17. N-(2-hydroxyethyl)-3-[2-[3-(trifluoromethyl)benzyl]-1-benzofuran-4-yl]benzamide, or a salt there of.

18. N-(2-hydroxyethyl)-3-[2-[3-(trifluoromethyl)benzyl]-1-benzofuran-4-yl]benzamide.

19. 3-[2-(3-chloro-4-fluorobenzyl)-2H-indazol-4-yl]-N-(2-cyanoethyl)benzamide, or a salt thereof.

20. N-(2-cyanoethyl)-3-{1-methyl-2-[3-(trifluoromethyl)benzyl]-1H-benzimidazol-4-yl}benzamide, or a salt thereof.

21. N-(2-methoxyethyl)-3-[1-methyl-2-[3-(trifluoromethyl)phenoxy]-1H-benzimidazol-4-yl]benzamide, or a salt thereof.

22. 3-[2-[[3-(trifluoromethyl)phenoxy]methyl]-1-benzothiophen-7-yl]benzamide, or a salt thereof.

23. 3-[2-[[3-(trifluoromethyl)phenoxy]methyl]-1-benzothiophen-7-yl]benzamide.

24. N-(2-hydroxyethyl)-3-[3-methyl-2-[3-(trifluoromethyl)benzyl]-1-benzothiophen-7-yl]benzamide, or a salt thereof.

25. N-(2-hydroxyethyl)-3-[3-methyl-2-[3-(trifluoromethyl)benzyl]-1-benzothiophen-7-yl]benzamide.

26. N-(2-hydroxyethyl)-2-{2-[3-(trifluoromethyl)benzyl]-1-benzothiophen-7-yl}pyridine-4-carboxamide, or a salt thereof.

27. N-(2-amino-2-oxoethyl)-3-[4-fluoro-2-[3-(trifluoromethyl)benzyl]-1-benzothiophen-7-yl]benzamide, or a salt thereof.

28. N-(2-amino-2-oxoethyl)-3-[4-fluoro-2-[3-(trifluoromethyl)benzyl]-1-benzothiophen-7-yl]benzamide.

29. N-(2-amino-2-oxoethyl)-3-[2-[3-(trifluoromethyl)benzyl]-1,3-benzothiazol-4-yl]benzamide, or a salt thereof.

30. 3-[2-(3-chloro-5-fluorobenzyl)-1-benzothiophen-7-yl]-N-[2-(1-methylethoxy)ethyl]benzamide, or a salt thereof.

31. 3-[2-(3-chloro-5-fluorobenzyl)-1-benzothiophen-7-yl]-N-[2-(1-methylethoxy)ethyl]benzamide.

32. A prodrug of the compound according to claim 1 or 2.

33. A pharmaceutical agent comprising:
the compound according to claim 1 or 2 or the prodrug according to claim 32.

34. The pharmaceutical agent according to claim 33 which is a GPR52 activating agent.

35. The pharmaceutical agent according to claim 34 which is a preventive or therapeutic agent for schizophrenia.

36. A GPR52 activating agent comprising a compound represented by formula (I₀): 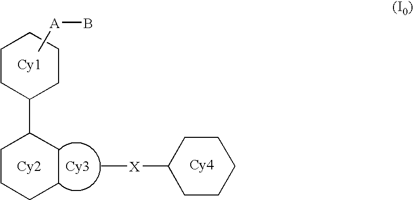
wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
a halogen atom,
a cyano group,
a hydroxy group,
a hydrocarbon-oxy group which may have one or more substituents,
a chain hydrocarbon group which may have one or more substituents,
a heterocyclic group which may have one or more substituents,
an amino group which may have one or more substituents,
an acyl group, and
a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR—, or —S(O)ₘ—,
R^{b} represents a hydrogen atom or a substituent,
m represents an integer of 0 to 2,
ring Cy4 represents a six-membered aromatic ring which may have one or more substituents;
with the proviso that
N-(3-(2-((4-chloro-2-methoxy-6-methylphenyl)amino)-1-methyl-1H-benzoimidazol-7-yl) phenyl)acetamide) is excluded;
or a salt thereof or a prodrug thereof.

37. The GPR52 activating agent according to claim 36 which is a preventive or therapeutic agent for schizophrenia.

38. A method of activating GPR52 comprising administrating an effective amount of a compound of formula (I₀): 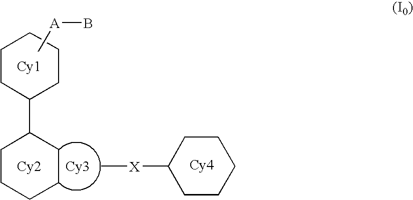
wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
a halogen atom,
a cyano group,
a hydroxy group,
a hydrocarbon-oxy group which may have one or more substituents,
a chain hydrocarbon group which may have one or more substituents,
a heterocyclic group which may have one or more substituents,
an amino group which may have one or more substituents,
an acyl group, and
a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
ring Cy4 represents a six-membered aromatic ring which may have one or more substituents;
with the proviso that
N-(3 -(2-((4-chloro-2-methoxy-6-methylphenyl)amino)-1-methyl-1H-benzoimidazol-7-yl) phenyl)acetamide) is excluded;
or a salt thereof or a prodrug thereof to the subject.

39. A method of treating or preventing schizophrenia comprising administrating an effective amount of a compound of formula (I₀): 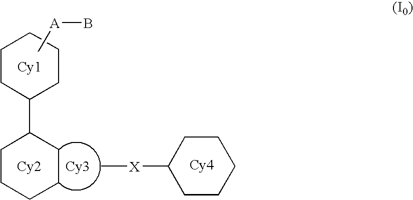
wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
a halogen atom,
a cyano group,
a hydroxy group,
a hydrocarbon-oxy group which may have one or more substituents,
a chain hydrocarbon group which may have one or more substituents,
a heterocyclic group which may have one or more substituents,
an amino group which may have one or more substituents,
an acyl group, and
a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
Cy4 represents a six-membered aromatic ring which may have one or more substituents;
with the proviso that
N-(3-(2-((4-chloro-2-methoxy-6-methylphenyl)amino)-1-methyl-1H-benzoimidazol-7-yl) phenyl)acetamide) is excluded;
or a salt thereof or a prodrug thereof to the subject.

40. Use of a compound represented by formula (I₀): 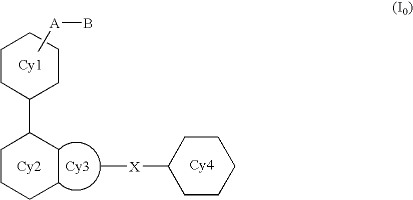
wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
a halogen atom,
a cyano group,
a hydroxy group,
a hydrocarbon-oxy group which may have one or more substituents,
a chain hydrocarbon group which may have one or more substituents,
a heterocyclic group which may have one or more substituents,
an amino group which may have one or more substituents,
an acyl group, and
a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R represents a hydrogen atom or a substituent,
m represents an integer of 0 to 2,
ring Cy4 represents a six-membered aromatic ring which may have one or more substituents;
with the proviso that
N-(3-(2-((4-chloro-2-methoxy-6-methylphenyl)amino)-1-methyl-1H-benzoimidazol-7-yl) phenyl)acetamide) is excluded;
or a salt thereof or a prodrug thereof
in the manufacture of a GPR52 activating agent.

41. Use of a compound represented by formula (I₀): 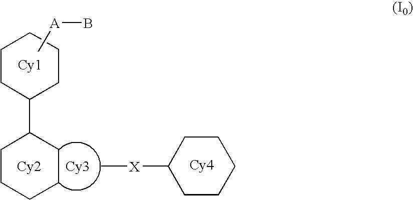
wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
a halogen atom,
a cyano group,
a hydroxy group,
a hydrocarbon-oxy group which may have one or more substituents,
a chain hydrocarbon group which may have one or more substituents,
a heterocyclic group which may have one or more substituents,
an amino group which may have one or more substituents,
an acyl group, and
a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
ring Cy4 represents a six-membered aromatic ring which may have one or more substituents;
with the proviso that
N-(3-(2-((4-chloro-2-methoxy-6-methylphenyl)amino)-1-methyl-1H-benzoimidazol-7-yl) phenyl)acetamide) is excluded;
or a salt thereof or a prodrug thereof
in the manufacture of a preventive or therapeutic of schizophrenia.

42. A compound represented by formula (I₀): 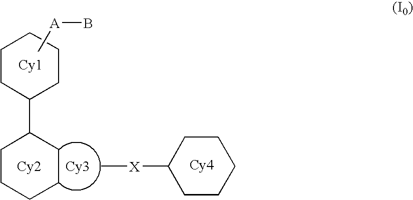
wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
a halogen atom,
a cyano group,
a hydroxy group,
a hydrocarbon-oxy group which may have one or more substituents,
a chain hydrocarbon group which may have one or more substituents,
a heterocyclic group which may have one or more substituents,
an amino group which may have one or more substituents,
an acyl group, and
a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or S(O)ₘ—,
R^{b} represents a hydrogen atom or a substituent,
m represents an integer of 0 to 2,
ring Cy4 represents a six-membered aromatic ring which may have one or more substituents;
with the proviso that
N-(3-(2-((4-chloro-2-methoxy-6-methylphenyl)amino)-1-methyl-1H-benzoimidazol-7-yl) phenyl)acetamide) is excluded;
or a salt thereof or a prodrug thereof
for use in activating GPR52.

43. A compound represented by formula (I₀): 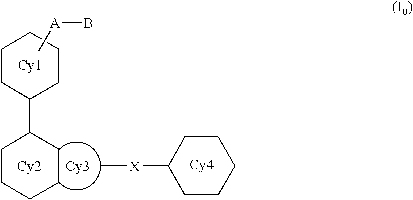
wherein
A represents —CONR^{a}— or —NR^{a}CO—;
R^{a} represents a hydrogen atom or a substituent;
B represents a hydrogen atom or a substituent;
or alternatively, when A is —CONR^{a}—, R^{a} and B may form together with an adjacent nitrogen atom a nitrogen-containing heterocyclic group which may have one or more substituents;
or further alternatively, when A is —CONR^{a}—, B may bond to the carbon atom adjacent to the carbon atom to which -A-B is attached to form a five- or six-membered ring which may have one or more substituents;
ring Cy1 represents a six-membered aromatic ring which may have one or more substituents in addition to a group represented by -A-B;
ring Cy2 represents a six-membered ring which may have one or more substituents selected from
a halogen atom,
a cyano group,
a hydroxy group,
a hydrocarbon-oxy group which may have one or more substituents,
a chain hydrocarbon group which may have one or more substituents,
a heterocyclic group which may have one or more substituents,
an amino group which may have one or more substituents,
an acyl group, and
a carboxy group which may be esterified,
ring Cy3 represents a five- or six-membered ring which may have one or more substituents,
X represents a C₁₋₂ alkylene which may be substituted with hydroxy, —Y—, Y—CH₂—, or —CH₂—Y—,
Y represents —O—, —NR^{b}—, or —S(O)ₘ—,
R^{b} represents a hydrogen atom or a substituent,
m represents an integer of 0 to 2,
ring Cy4 represents a six-membered aromatic ring which may have one or more substituents;
with the proviso that
N-(3-(2-((4-chloro-2-methoxy-6-methylphenyl)amino)-1-methyl-1H-benzoimidazol-7-yl) phenyl)acetamide) is excluded;
or a salt thereof or a prodrug thereof
for use in treating or preventing schizophrenia.
